(19) United States
(12) Patent Application Publication
(10) Pub. No.: US 2026/0258053 A1
(43) Pub. Date: Sep. 3, 2026

(54) BENZAMIDE ARYL ETHER ANALOGS COMPRISING AN AMINE LINKER AS INHIBITORS OF THE MENIN-MLL INTERACTION

(71) Applicant: Syndax Pharmaceuticals, Inc., New York, NY (US)

(72) Inventors: Gerard M. MCGEEHAN, Newtown Square, PA (US); William H. MILLER, Collegeville, PA (US); Swapan K. SAMANTA, Hyderabad (IN); Virsinha REDDY, Hyderabad (IN)

(21) Appl. No.: 19/553,044

(22) Filed: Feb. 27, 2026

Related U.S. Application Data
(60) Provisional application No. 63/763,968, filed on Feb. 27, 2025.
(60) Provisional application No. 63/950,234, filed on Dec. 29, 2025.

(51) Int. Cl. C07D 498/04; A61K 31/506; A61K 31/5377; A61K 31/5383; A61K 31/553; A61P 35/02; C07B 59/00; C07D 401/14; C07D 403/14; C07D 413/14; C07D 471/04; C07D 487/04; C07D 519/00

(52) U.S. Cl. CPC C07D 498/04; A61K 31/506; A61K 31/5377; A61K 31/5383; A61K 31/553; A61P 35/02; C07B 59/002; C07D 401/14; C07D 403/14; C07D 413/14; C07D 471/04; C07D 487/04; C07D 519/00

(57) ABSTRACT

The Present disclosure is directed to aryl ether analogs comprising an amine linker, of Formula I, a stereoisomer, or a pharmaceutically acceptable salt thereof, as inhibitors of the interaction of menin with MLL and MLL fusion proteins, pharmaceutical compositions containing the same, and their use in the treatment of cancer and other diseases mediated by the menin-MLL interaction.

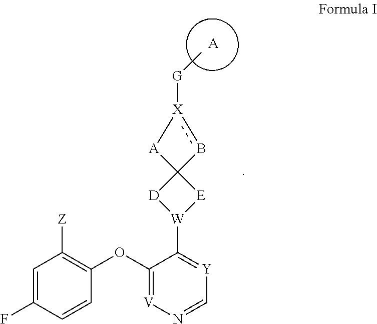

### RELATED APPLICATIONS

This application claims priority to, and the benefit of, U.S. Provisional Application No. 63/763,968, filed Feb. 27, 2025, and U.S. Provisional Application No. 63/950,234, filed Dec. 29, 2025, the entire contents of each of which are incorporated herein by reference.

### BACKGROUND

The mixed-lineage leukemia (MLL), also known as KMT2A, gene encodes a protein that is a histone methyltransferase that is mutated in clinically and biologically distinctive subsets of acute leukemia. Rearranged mixed lineage leukemia (MLL-r) involves recurrent translocations of the 11q23 chromosome locus which lead to an aggressive form of acute leukemia with limited therapeutic options. These translocations target the MLL gene creating an oncogenic fusion protein comprising the amino-terminus of MLL fused in frame with more than 60 different fusion protein partners. Menin, a ubiquitously expressed, nuclear protein encoded by the multiple endocrine neoplasia type 1 (MEN1) tumor suppressor gene, has a high affinity binding interaction with MLL fusion proteins and is an essential co-factor of oncogenic MLL-r fusion proteins. Disruption of this interaction leads to selective growth inhibition and apoptosis of MLL-r leukemia cells both in vitro and in vivo.

The interaction between menin and MLL or MLL fusion proteins is an attractive target for therapeutic intervention, and there is a need for novel agents that inhibit the menin-MLL interaction for the treatment of various diseases and conditions, including leukemia, other cancers, and diabetes.

### SUMMARY

In one aspect, the present disclosure is directed to a compound of Formula I,

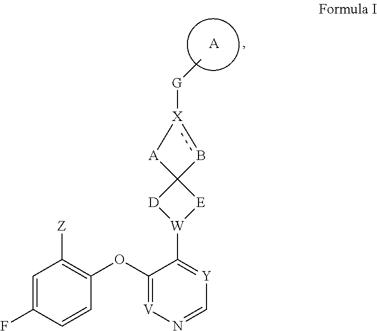

a stereoisomer a pharmaceutically acceptable salt thereof, wherein:
[symbol] denotes a single bond or a double bond, as valency permits;
A, B, D, and E are each independently selected from —O—, —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent, O, CH₂, or NH, wherein when G is absent, X is connected with a nitrogen of Ring A;
when G is O, CH₂, or NH, G is connected with the nitrogen of Ring A; W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
is Cy², halo, C₁₋₆ alkyl, C₁₋₆ haloalkyl, C₁₋₆ cyanoalkyl, C₂₋₆ alkenyl, C₂₋₆ alkynyl, CN, NO₂, OR^{a3}, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3}, NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, and P(O)R^{c3}R^{d3} wherein said C₁₋₆ alkyl, C₁₋₆ haloalkyl, C₁₋₆ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3} NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH;
R^{z} is H, C₁₋₆ alkyl, or phenyl;
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{e5}C(O)OR^{a5}, NR^{e5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as}; each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{a3}, R^{b3}, R^{c3}, R^{d3}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e3} and R^{e5} is independently selected from H, C₁₋₆ alkyl, and CN;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula II,

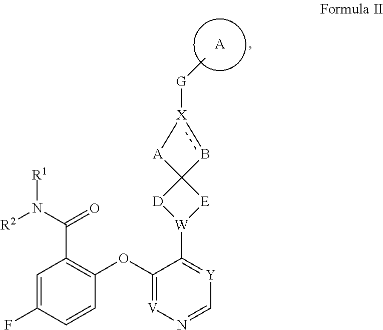

a stereoisomer, or a pharmaceutically acceptable salt thereof, wherein:
[symbol] denotes a single bond or a double bond, as valency permits;
A, B, D, and E are each independently selected from —O—, —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH;
R^{z} is H, C₁₋₆ alkyl, or phenyl;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a}
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula II-a,

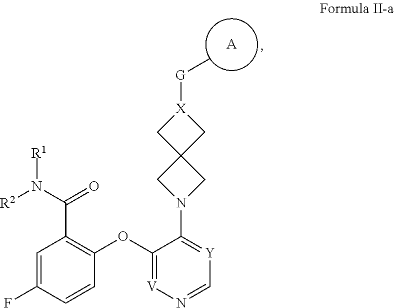

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a}
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula II-b,

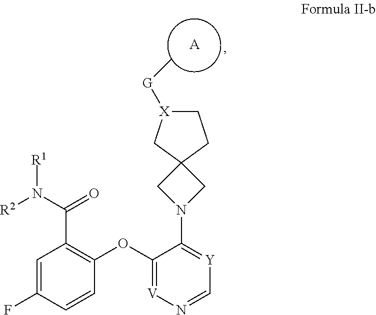

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a}
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula II-c,

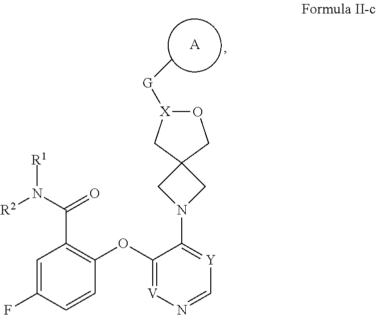

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as}; each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula II-d,

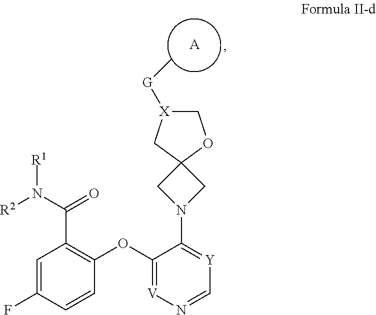

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present disclosure is directed to a compound of Formula II-e,

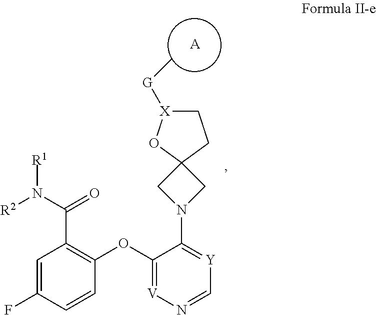

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In one aspect, the present application relates to a pharmaceutical composition comprising a compound of the application, or a pharmaceutically acceptable salt, and a pharmaceutically acceptable carrier.

In one aspect, the present application relates to a pharmaceutical composition comprising a therapeutically effective amount of a compound of the application, or a pharmaceutically acceptable salt, and a pharmaceutically acceptable carrier.

In one aspect, the present application relates to a pharmaceutical composition comprising a compound of the application, and a pharmaceutically acceptable carrier.

In one aspect, the present application relates to a pharmaceutical composition comprising a therapeutically effective amount of a compound of the application, and a pharmaceutically acceptable carrier.

The present disclosure further provides a use of a compound of Formula I, II, II-a, II-b, II-c, II-d, or II-e, or a stereoisomer, or a pharmaceutically acceptable salt thereof in a method of treating cancer in a patient in need thereof.

The present disclosure further provides a compound of Formula I, II, II-a, II-b, II-c, II-d, or II-e, or a stereoisomer, or a pharmaceutically acceptable salt thereof for use in a method of treating cancer in a patient in need thereof.

The present disclosure further provides a compound of Formula I, II, II-a, II-b, II-c, II-d, or II-e, or a stereoisomer, or a pharmaceutically acceptable salt thereof for use in the manufacture of a medicament for the treatment of cancer in a patient in need thereof.

The present disclosure further provides a method of inhibiting the interaction between menin and MLL comprising contacting the menin and MLL with an effective amount of a compound of Formula I, II, II-a, II-b, II-c, II-d, or II-e, or a stereoisomer, or a pharmaceutically acceptable salt thereof.

The present disclosure further provides a method of treating cancer in a patient comprising administering to the patient a therapeutically effective amount of a compound of Formula I, II, II-a, II-b, II-c, II-d, or II-e, a stereoisomer, or a pharmaceutically acceptable salt thereof.

The present disclosure further provides a method of inhibiting the interaction between menin and MLL comprising contacting the menin and MLL with a compound of Formula I, II, II-a, II-b, II-c, II-d, or II-e, a stereoisomer, or a pharmaceutically acceptable salt thereof.

The present disclosure further provides a method of treating cancer in a patient comprising administering to the patient an effective amount of a compound of Formula I, II, II-a, II-b, II-c, II-d, or II-e, a stereoisomer, or a pharmaceutically acceptable salt thereof.

The present disclosure further provides a method of inhibiting the interaction between menin and MLL comprising contacting the menin and MLL with a compound of Formula I, II, II-a, II-b, II-c, II-d, or II-e, or a stereoisomer, or a pharmaceutically acceptable salt thereof.

The present disclosure further provides a method of inhibiting the interaction between menin and MLL comprising contacting the menin and MLL with a pharmaceutical composition comprising a compound of Formula I, II, II-a, II-b, II-c, II-d, or II-e, or a pharmaceutically acceptable salt thereof, and at least one pharmaceutically acceptable carrier.

The present disclosure further provides a method of treating cancer in a patient comprising administering to the patient a compound of Formula I, II, II-a, II-b, II-c, II-d, or II-e, a stereoisomer, or a pharmaceutically acceptable salt thereof.

The present disclosure further provides a method of treating cancer in a patient comprising administering to the patient a pharmaceutical composition comprising a compound of Formula I, II, II-a, II-b, II-c, II-d, or II-e, a stereoisomer, or a pharmaceutically acceptable salt thereof, and at least one pharmaceutically acceptable carrier.

The present disclosure further provides a pharmaceutical composition comprising a compound of Formula I, II, II-a, II-b, II-c, II-d, or II-e, a stereoisomer, or a pharmaceutically acceptable salt thereof, and at least one pharmaceutically acceptable carrier.

The present disclosure further provides a pharmaceutical composition comprising a salt or crystalline form of a compound of Formula I, II, II-a, II-b, II-c, II-d, or II-e, and at least one pharmaceutically acceptable carrier.

The present disclosure further provides a compound of Formula I, II, II-a, II-b, II-c, II-d, or II-e, wherein the compound is useful for the treatment of cancer and wherein the compound minimizes hERG binding.

In some embodiments, present disclosure further provides a method of preparing a compound herein according to a scheme of the present disclosure or synthetic description in the Examples.

In some embodiments, present disclosure further provides an intermediate useful in the preparation of any one of the compounds herein.

The details of the disclosure are set forth in the accompanying description below. Although methods and materials similar or equivalent to those described herein can be used in the practice or testing of the present application, illustrative methods and materials are now described. In the case of conflict, the present specification, including definitions, will control. In addition, the materials, methods, and examples are illustrative only and are not intended to be limiting. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### DETAILED DESCRIPTION

In some embodiments, the present disclosure is directed to a compound of Formula I,

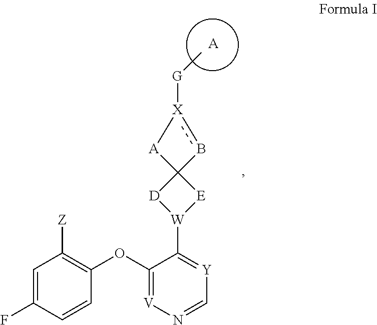

a stereoisomer, or a pharmaceutically acceptable salt thereof, wherein:
[symbol] denotes a single bond or a double bond, as valency permits;
A, B, D, and E are each independently selected from —O—, —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent, O, CH₂, or NH, wherein
   when G is absent, X is connected with a nitrogen of Ring A;
   when G is O, CH₂, or NH, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
Z is Cy², halo, C₁₋₆ alkyl, C₁₋₆ haloalkyl, C₁₋₆ cyanoalkyl, C₂₋₆ alkenyl, C₂₋₆ alkynyl, CN, NO₂, OR^{a3}, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3} NR^{c3}C(NR^{e3})NR^{c3}R^{d3} NR^{c3}R^{d3} NR^{c3}C(O)R^{b3}, NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, and P(O)R^{c3}R^{d3} wherein said C₁₋₆ alkyl, C₁₋₆ haloalkyl, C₁₋₆ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3} NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH;
R^{z} is H, C₁₋₆ alkyl, or phenyl;
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{e5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{a3}, R^{b3}, R^{c3}, R^{d3}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e3} and R^{e5} is independently selected from H, C₁₋₆ alkyl, and CN;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, when [symbol] is a double bond, X is C.

In some embodiments, the present disclosure is directed to a compound of Formula I, a stereoisomer, or a pharmaceutically acceptable salt thereof, wherein:
[symbol] denotes a single bond;
A, B, D, and E are each independently selected from —C(R^{A1})(R^{A2})—;
V is N or CH;
Y is N or CH;
Ring A is 4-18 membered heterocycloalkyl which has at least one nitrogen and optionally one or more other heteroatom ring members, and is optionally substituted with one or more R³;
G is CH₂;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
Z is Cy², halo, C₁₋₆ alkyl, C₁₋₆ haloalkyl, C₁₋₆ cyanoalkyl, C₂₋₆ alkenyl, C₂₋₆ alkynyl, CN, NO₂, OR^{a3}, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3}, NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, and P(O)R^{c3}R^{d3} wherein said C₁₋₆ alkyl, C₁₋₆ haloalkyl, C₁₋₆ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3} NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3};
each R^{A1} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
R^{z} is H, C₁₋₆ alkyl, or phenyl;
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{e5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs}
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{a3}, R^{b3}, R^{c3}, R^{d3}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e3} and R^{e5} is independently selected from H, C₁₋₆ alkyl, and CN;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula I, a stereoisomer, or a pharmaceutically acceptable salt thereof, wherein:
[symbol] denotes a single bond;
A, B, D, and E are each independently selected from —O—, —C(R^{A1})(R^{A2})—, —C(R^{A1})(R^{A2})—C(R^{A1})(R^{A2})—, and —C(R^{A1})(R^{A2})—O—, wherein no more than one of A, B, D, and E is —C(R^{A1})(R^{A2})—O—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
Z is Cy² or C(O)NR¹R²;
each R^{A1} is independently selected from absent, H, halo, C₁₋₆ alkyl, C₁₋₆ alkoxy, C₁₋₆ haloalkyl, C₁₋₆ haloalkoxy, CN, and OH;
each R^{A2} is independently selected from H, halo, C₁₋₆ alkyl, C₁₋₆ alkoxy, C₁₋₆ haloalkyl, or C₁₋₆ haloalkoxy, CN, and OH;
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, and OR^{a5};
R¹ is
   (a) H, C₁₋₆ alkyl, C₁₋₆ haloalkyl, C₁₋₆ alkoxy, C₂₋₆ alkenyl, C₂₋₆ alkynyl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{a5} is independently selected from H, C₁₋₆ alkyl, or C₁₋₆ haloalkyl;
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₁₋₆ alkoxy, wherein the C₁₋₆ alkyl, or C₁₋₆ alkoxy, is optionally substituted with one or more substituent independently selected from halo, O(R^{4a}′), oxo, CN, or N(R^{4a}′)(R^{4b}),
each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula I, a stereoisomer, or a pharmaceutically acceptable salt thereof, wherein:
[symbol] denotes a single bond;
A, B, D, and E are each independently selected from —C(R^{A1})(R^{A2})—;
V is N or CH;
Y is N or CH;
Ring A is 4-18 membered heterocycloalkyl which has at least one nitrogen and optionally one or more other heteroatom ring members, and is optionally substituted with one or more R³;
G is CH₂;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
Z is Cy² or C(O)NR¹R²;
each R^{A1} is independently selected from absent, H, halo, C₁₋₆ alkyl, C₁₋₆ alkoxy, C₁₋₆ haloalkyl, C₁₋₆ haloalkoxy, CN, and OH;
each R^{A2} is independently selected from H, halo, C₁₋₆ alkyl, C₁₋₆ alkoxy, C₁₋₆ haloalkyl, or C₁₋₆ haloalkoxy, CN, and OH;
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, and OR^{a5};
R¹ is
   C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
each R^{a5} is independently selected from H, C₁₋₆ alkyl, or C₁₋₆ haloalkyl;
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, or C₁₋₆ alkoxy, wherein the C₁₋₆ alkyl or C₁₋₆ alkoxy is optionally substituted with one or more substituent independently selected from halo, O(R^{4a}′), oxo, CN, or N(R^{4a}′)(R^{4b}′);
each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula I, a stereoisomer, or a pharmaceutically acceptable salt thereof, wherein:
[symbol] denotes a single bond;
A, B, D, and E are each independently selected from —O—, —C(R^{A1})(R^{A2})—, —C(R^{A1})(R^{A2})—C(R^{A1})(R^{A2})—, and —C(R^{A1})(R^{A2})—O—, wherein no more than one of A, B, D, and E is —C(R^{A1})(R^{A2})—O—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
W is N;
X is CR^{X}, wherein R^{X} is H;
Z is C(O)NR¹R²;
each R^{A1} is independently selected from H;
each R^{A2} is independently selected from H;
R¹ is
   (a) C₁₋₆ alkyl, C₁₋₆ haloalkyl, or 3-12 membered heterocycloalkyl, wherein said C₁₋₆ alkyl, C₁₋₆haloalkyl, or 3-12 membered heterocycloalkyl, are each optionally substituted with 1 substituent independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is C₁₋₆ alkyl; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from C₁₋₆ alkyl;
each R³ is independently H, oxo, N(R^{4a})(R^{4b}), C₁₋₆ alkyl, or C(O)O—C(R^{4a})₂—OC(O)(R^{4a}), wherein the C₁₋₆ alkyl, is optionally substituted with one or more R^{3a};
each R^{3a} is independently N(R^{4a})(R^{4b});
each R^{4a} is independently H or C₁₋₆ alkyl; and
each R^{4b} is independently H.

In some embodiments, the present disclosure is directed to a compound of Formula I, a stereoisomer, or a pharmaceutically acceptable salt thereof, wherein:
[symbol] denotes a single bond;
A, B, D, and E are each independently selected from —C(R^{A1})(R^{A2})—;
V is N or CH;
Y is N or CH;
Ring A is 4-18 membered heterocycloalkyl which has at least one nitrogen and optionally one or more other heteroatom ring members, and is optionally substituted with one or more R³;
G is CH₂;
W is N;
X is CR^{X}, wherein R^{X} is H;
Z is C(O)NR¹R²;
each R^{A1} is independently selected from H;
each R^{A2} is independently selected from H;
R¹ is
   C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl;
each R³ is independently H, oxo, N(R^{4a})(R^{4b}), C₁₋₆ alkyl, or C(O)O—C(R^{4a})₂—OC(O)(R^{4a}) wherein the C₁₋₆ alkyl is optionally substituted with one or more R^{3a};
each R^{3a} is independently N(R^{4a})(R^{4b});
each R^{4a} is independently H or C₁₋₆ alkyl; and
each R^{4b} is independently H.

In some embodiments, the present disclosure is directed to a compound of Formula II,

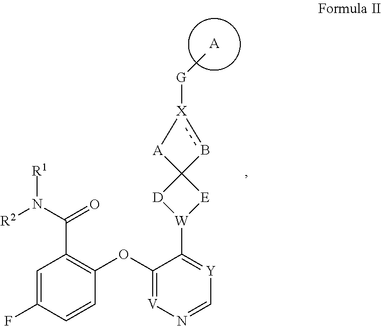

a stereoisomer, or a pharmaceutically acceptable salt thereof, wherein:
[symbol] denotes a single bond or a double bond, as valency permits;
A, B, D, and E are each independently selected from —O—, —C(R^{A1})(R^{A2})—, —C(R^{A1})(R^{A2})—C(R^{A1})(R^{A2})—, —C(RAl)(R^{A2})—O—, —C(RAl)(R^{A2})NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH;
R^{z} is H, C₁₋₆ alkyl, or phenyl;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II, a stereoisomer, or a pharmaceutically acceptable salt thereof, wherein:
[symbol] denotes a single bond;
A, B, D, and E are each independently selected from —C(R^{A1})(R^{A2})—;
V is N or CH;
Y is N or CH;
Ring A is 4-18 membered heterocycloalkyl which has at least one nitrogen and optionally one or more other heteroatom ring members, and is optionally substituted with one or more R³;
G is CH₂ and G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
R¹ is
   C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II, a stereoisomer, or a pharmaceutically acceptable salt thereof, wherein:
[symbol] denotes a single bond;
A, B, D, and E are each independently selected from —O—, —C(R^{A1})(R^{A2})—, —C(R^{A1})(R^{A2})—C(R^{A1})(R^{A2})—, and —C(R^{A1})(R^{A2})—O—, wherein no more than one of A, B, D, and E is —C(R^{A1})(R^{A2})—O—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₆ alkyl, C₁₋₆ alkoxy, C₁₋₆ haloalkyl, C₁₋₆ haloalkoxy, CN, and OH;
each R^{A2} is independently selected from H, halo, C₁₋₆ alkyl, C₁₋₆ alkoxy, C₁₋₆ haloalkyl, or C₁₋₆ haloalkoxy, CN, and OH;
R¹ is
   (a) H, C₁₋₆ alkyl, C₁₋₆ haloalkyl, C₁₋₆ alkoxy, C₂₋₆ alkenyl, C₂₋₆ alkynyl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₁₋₆ alkoxy, wherein the C₁₋₆ alkyl, or C₁₋₆ alkoxy, is optionally substituted with one or more substituent independently selected from halo, O(R^{4a}′), oxo, CN, or N(R^{4a}′)(R^{4b}′) each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II, a stereoisomer, or a pharmaceutically acceptable salt thereof, wherein:
[symbol] denotes a single bond;
A, B, D, and E are each independently selected from —C(R^{A1})(R^{A2})—;
V is N or CH;
Y is N or CH;
Ring A is 4-18 membered heterocycloalkyl which has at least one nitrogen and optionally one or more other heteroatom ring members, and is optionally substituted with one or more R³;
G is CH₂ and G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₆ alkyl, C₁₋₆ alkoxy, C₁₋₆ haloalkyl, C₁₋₆ haloalkoxy, CN, and OH;
each R^{A2} is independently selected from H, halo, C₁₋₆ alkyl, C₁₋₆ alkoxy, C₁₋₆ haloalkyl, or C₁₋₆ haloalkoxy, CN, and OH;
R¹ is
   C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₁₋₆ alkoxy, wherein the C₁₋₆ alkyl, or C₁₋₆ alkoxy, is optionally substituted with one or more substituent independently selected from halo, O(R^{4a}′), oxo, CN, or N(R^{4a}′)(R^{4b}′);
each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II, a stereoisomer, or a pharmaceutically acceptable salt thereof, wherein:
[symbol] denotes a single bond;
A, B, D, and E are each independently selected from —O—, —C(R^{A1})(R^{A2})—, —C(R^{A1})(R^{A2})—C(R^{A1})(R^{A2})—, and —C(R^{A1})(R^{A2})—O—, wherein no more than one of A, B, D, and E is —C(R^{A1})(R^{A2})—O—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
W is N;
X is CR^{X}, wherein R^{X} is H;
each R^{A1} is independently selected from H;
each R^{A2} is independently selected from H;
R¹ is
   (a) C₁₋₆ alkyl, C₁₋₆ haloalkyl, or 3-12 membered heterocycloalkyl, wherein said C₁₋₆ alkyl, C₁₋₆haloalkyl, or 3-12 membered heterocycloalkyl, are each optionally substituted with 1 substituent independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is C₁₋₆ alkyl; or
   (c) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from C₁₋₆ alkyl;
each R³ is independently H, oxo, N(R^{4a})(R^{4b}), C₁₋₆ alkyl, or C(O)O—C(R^{4a})₂—OC(O)(R^{4a}), wherein the C₁₋₆ alkyl, is optionally substituted with one or more R^{3a};
each R^{3a} is independently N(R^{4a})(R^{4b});
each R^{4a} is independently H or C₁₋₆ alkyl; and
each R^{4b} is independently H.

In some embodiments, the present disclosure is directed to a compound of Formula II, a stereoisomer, or a pharmaceutically acceptable salt thereof, wherein:
[symbol] denotes a single bond;
A, B, D, and E are each independently selected from —C(R^{A1})(R^{A2})—;
V is N or CH;
Y is N or CH;
Ring A is 4-18 membered heterocycloalkyl which has at least one nitrogen and optionally one or more other heteroatom ring members, and is optionally substituted with one or more R³;
G is CH₂ and G is connected with the nitrogen of Ring A;
W is N;
X is CR^{X}, wherein R^{X} is H;
each R^{A1} is independently selected from H;
each R^{A2} is independently selected from H;
R¹ is
   C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl;
each R³ is independently H, oxo, N(R^{4a})(R^{4b}), C₁₋₆ alkyl, or C(O)O—C(R^{4a})₂—OC(O)(R^{4a}) wherein the C₁₋₆ alkyl, is optionally substituted with one or more R^{3a};
each R^{3a} is independently N(R^{4a})(R^{4b});
each R^{4a} is independently H or C₁₋₆ alkyl; and
each R^{4b} is independently H.

In some embodiments, the present disclosure is directed to a compound of Formula II-a,

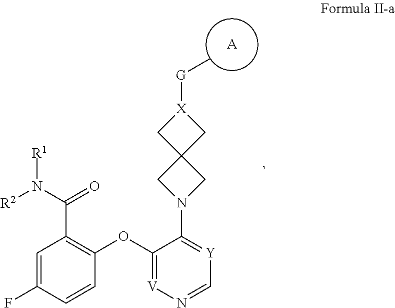

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-a, a stereoisomer, or a pharmaceutically acceptable salt thereof, wherein:
V is N or CH;
Y is N or CH;
Ring A is 4-18 membered heterocycloalkyl which has at least one nitrogen and optionally one or more other heteroatom ring members, and is optionally substituted with one or more R³;
G is CH₂ and G is connected with the nitrogen of Ring A;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
R¹ is
   C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-a, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₁₋₆ alkoxy, wherein the C₁₋₆ alkyl, or C₁₋₆ alkoxy, is optionally substituted with one or more substituent independently selected from halo, O(R^{4a}′), oxo, CN, or N(R^{4a}′)(R^{4b}′) each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-a, a stereoisomer, or a pharmaceutically acceptable salt thereof, wherein:
V is N or CH;
Y is N or CH;
Ring A is 4-18 membered heterocycloalkyl which has at least one nitrogen and optionally one or more other heteroatom ring members, wherein the 4-18 membered heterocycloalkyl or the 5- to 10-membered heteroaryl is optionally substituted with one or more R³;
G is CH₂ and G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₁₋₆ alkoxy, wherein the C₁₋₆ alkyl or C₁₋₆ alkoxy is optionally substituted with one or more substituent independently selected from halo, O(R^{4a}′), oxo, CN, or N(R^{4a}′)(R^{4b}′) each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-a, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is CR^{X}, wherein R^{X} is H;
R¹ is
   (a) C₁₋₆ alkyl, C₁₋₆ haloalkyl, or 3-12 membered heterocycloalkyl, wherein said C₁₋₆ alkyl, C₁₋₆haloalkyl, or 3-12 membered heterocycloalkyl, are each optionally substituted with 1 substituent independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is C₁₋₆ alkyl; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from C₁₋₆ alkyl;
each R³ is independently H, oxo, N(R^{4a})(R^{4b}), C₁₋₆ alkyl, or C(O)O—C(R^{4a})₂—OC(O)(R^{4a}) wherein the C₁₋₆ alkyl, is optionally substituted with one or more R^{3a};
each R^{3a} is independently N(R^{4a})(R^{4b});
each R^{4a} is independently H or C₁₋₆ alkyl; and
each R^{4b} is independently H.

In some embodiments, the present disclosure is directed to a compound of Formula II-a, a stereoisomer, or a pharmaceutically acceptable salt thereof, wherein:
V is N or CH;
Y is N or CH;
Ring A is 4-18 membered heterocycloalkyl which has at least one nitrogen and optionally one or more other heteroatom ring members, and is optionally substituted with one or more R³;
G is CH₂ and G is connected with the nitrogen of Ring A;
X is CR^{X}, wherein R^{X} is H;
R¹ is
   C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl;
each R³ is independently H, oxo, N(R^{4a})(R^{4b}), C₁₋₆ alkyl, or C(O)O—C(R^{4a})₂—OC(O)(R^{4a}) wherein the C₁₋₆ alkyl, is optionally substituted with one or more R^{3a};
each R^{3a} is independently N(R^{4a})(R^{4b});
each R^{4a} is independently H or C₁₋₆ alkyl; and
each R^{4b} is independently H.

In some embodiments, the present disclosure is directed to a compound of Formula II-b,

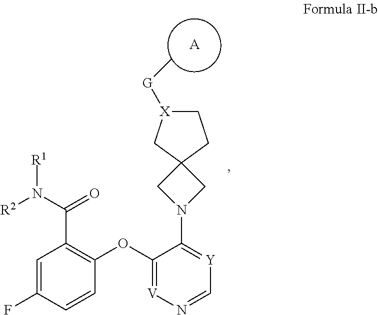

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-b, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₁₋₆ alkoxy, wherein the C₁₋₆ alkyl or C₁₋₆ alkoxy is optionally substituted with one or more substituent independently selected from halo, O(R^{4a}′), oxo, CN, and N(R^{4a}′)(R^{4b}′);
each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-b, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is CR^{X}, wherein R^{X} is H;
R¹ is
   (a) C₁₋₆ alkyl, C₁₋₆ haloalkyl, or 3-12 membered heterocycloalkyl, wherein said C₁₋₆ alkyl, C₁₋₆haloalkyl, or 3-12 membered heterocycloalkyl, are each optionally substituted with 1 substituent independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is C₁₋₆ alkyl; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl; or
R¹ and R² optionally form a 3- to 12-membered heterocycloalkyl with the nitrogen to which they are connected, wherein the heterocycloalkyl is optionally substituted with one or more C₁₋₆ alkyl;
each R^{g} is independently selected from C₁₋₆ alkyl;
each R³ is independently H, oxo, N(R^{4a})(R^{4b}), C₁₋₆ alkyl, or C(O)O—C(R^{4a})₂—OC(O)(R^{4a}), wherein the C₁₋₆ alkyl, is optionally substituted with one or more R^{3a};
each R^{3a} is independently N(R^{4a})(R^{4b});
each R^{4a} is independently H or C₁₋₆ alkyl; and
each R^{4b} is independently H.

In some embodiments, the present disclosure is directed to a compound of Formula II-c,

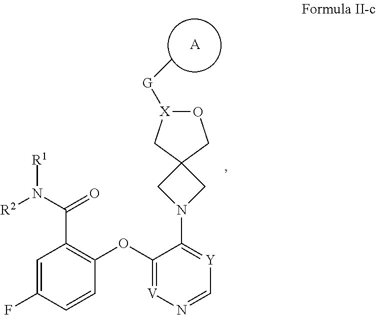

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}, N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-c, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₁₋₆ alkoxy, wherein the C₁₋₆ alkyl or C₁₋₆ alkoxy is optionally substituted with one or more substituent independently selected from halo, O(R^{4a}′), oxo, CN, and N(R^{4a}′)(R^{4b}′);
each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-c, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is CR^{X}, wherein R^{X} is H;
R¹ is
   (a) C₁₋₆ alkyl, C₁₋₆ haloalkyl, or 3-12 membered heterocycloalkyl, wherein said C₁₋₆ alkyl, C₁₋₆haloalkyl, or 3-12 membered heterocycloalkyl, are each optionally substituted with 1 substituent independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is C₁₋₆ alkyl; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl; or
R¹ and R² optionally form a 3- to 12-membered heterocycloalkyl with the nitrogen to which they are connected, wherein the heterocycloalkyl is optionally substituted with one or more C₁₋₆ alkyl;
each R^{g} is independently selected from C₁₋₆ alkyl;
each R³ is independently H, oxo, N(R^{4a})(R^{4b}), C₁₋₆ alkyl, or C(O)O—C(R^{4a})₂—OC(O)(R^{4a}) wherein the C₁₋₆ alkyl, is optionally substituted with one or more R^{3a};
each R^{3a} is independently N(R^{4a})(R^{4b});
each R^{4a} is independently H or C₁₋₆ alkyl; and
each R^{4b} is independently H.

In some embodiments, the present disclosure is directed to a compound of Formula II-d,

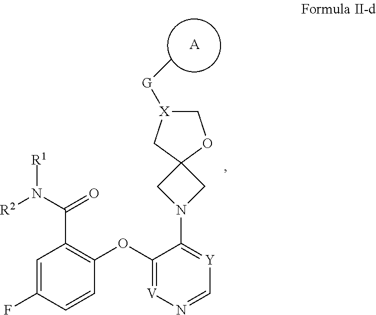

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-d, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₁₋₆ alkoxy, wherein the C₁₋₆ alkyl or C₁₋₆ alkoxy is optionally substituted with one or more substituent independently selected from halo, O(R^{4a}′), oxo, CN, and N(R^{4a}′)(R^{4b}′);
each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-d, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is CR^{X}, wherein R^{X} is H;
R¹ is
   (a) C₁₋₆ alkyl, C₁₋₆ haloalkyl, or 3-12 membered heterocycloalkyl, wherein said C₁₋₆ alkyl, C₁₋₆haloalkyl, or 3-12 membered heterocycloalkyl, are each optionally substituted with 1 substituent independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is C₁₋₆ alkyl; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl; or
R¹ and R² optionally form a 3- to 12-membered heterocycloalkyl with the nitrogen to which they are connected, wherein the heterocycloalkyl is optionally substituted with one or more C₁₋₆ alkyl;
each R^{g} is independently selected from C₁₋₆ alkyl;
each R³ is independently H, oxo, N(R^{4a})(R^{4b}), C₁₋₆ alkyl, or C(O)O—C(R^{4a})₂—OC(O)(R^{4a}), wherein the C₁₋₆ alkyl, is optionally substituted with one or more R^{3a};
each R^{3a} is independently N(R^{4a})(R^{4b});
each R^{4a} is independently H or C₁₋₆ alkyl; and
each R^{4b} is independently H.

In some embodiments, the present disclosure is directed to a compound of Formula II-e,

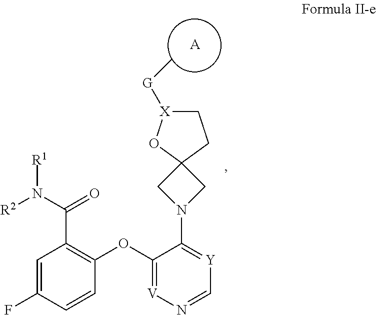

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}, N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-e, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is C₁₋₆ alkyl, C₁₋₆ haloalkyl, C₁₋₆ alkoxy, C₂₋₆ alkenyl, or C₂₋₆ alkynyl, C₆₋₁₀ aryl, or 3-12 membered heterocycloalkyl, wherein said C₁₋₆ alkyl, C₁₋₆ haloalkyl, C₁₋₆ alkoxy, C₂₋₆ alkenyl, C₂₋₆ alkynyl, or 3-12 membered heterocycloalkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, and C₁₋₆ haloalkoxy;
each R³ is independently H, oxo, N(R^{4a})(R^{4b}), C₁₋₆ alkyl, C₂₋₆ alkenyl, C₂₋₆ alkynyl, C₁₋₆ alkoxy, or C(O)O—C(R^{4a})₂—OC(O)(R^{4a}), wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₁₋₆ alkoxy, wherein the C₁₋₆ alkyl or C₁₋₆ alkoxy is optionally substituted with one or more substituent independently selected from halo, O(R^{4a}′), oxo, CN, and N(R^{4a}′)(R^{4b}′);
each R^{4a} is independently H, CN, or C₁₋₆ alkyl optionally substituted with one or more halo, C₆₋₁₀ aryl, or N(R^{4a}′)(R^{4b}′);
each R^{4b} is independently H or C₁₋₆ alkyl;
each R^{4a}′ and R^{4b}′ is independently H or C₁₋₆ alkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

In some embodiments, the present disclosure is directed to a compound of Formula II-e, a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein
   when G is absent, X is connected with a nitrogen of Ring A;
   when G is CH₂, G is connected with the nitrogen of Ring A;
X is CR^{X}, wherein R^{X} is H;
R¹ is
   (a) C₁₋₆ alkyl, C₁₋₆ haloalkyl, or 3-12 membered heterocycloalkyl, wherein said C₁₋₆ alkyl, C₁₋₆haloalkyl, or 3-12 membered heterocycloalkyl, are each optionally substituted with 1 substituent independently selected from R^{g};
   (b) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is C₁₋₆ alkyl; or
   (c) C₃₋₁₂ cycloalkyl substituted with one or more 3- to 12-membered heterocycloalkyl which has at least one nitrogen and optionally one or more other heteroatom ring members and wherein each of the C₃₋₁₂ cycloalkyl and the 3- to 12-membered heterocycloalkyl is optionally substituted with one or more R^{1cs};
   each R^{1cs} is independently C₁₋₆ alkyl;
R² is C₁₋₆ alkyl; or
R¹ and R² optionally form a 3- to 12-membered heterocycloalkyl with the nitrogen to which they are connected, wherein the heterocycloalkyl is optionally substituted with one or more C₁₋₆ alkyl;
each R^{g} is independently selected from C₁₋₆ alkyl;
each R³ is independently H, oxo, N(R^{4a})(R^{4b}), C₁₋₆ alkyl, or C(O)O—C(R^{4a})₂—OC(O)(R^{4a}) wherein the C₁₋₆ alkyl, is optionally substituted with one or more R^{3a};
each R^{3a} is independently N(R^{4a})(R^{4b});
each R^{4a} is independently H or C₁₋₆ alkyl; and
each R^{4b} is independently H.

### EMBODIMENTS

For any of Formulae I, II, II-a, II-b, II-c, II-d, and II-e where applicable, the following embodiments are considered both alone and in conjunction with another where a stable compound is formed.

### Embodiments Covering A, B, D, and E

In some embodiments, A, B, D, and E are each independently selected from —O—, —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—.

In some embodiments, A, B, D, and E are each independently selected from —O—, —C(R^{A1})(R^{A2})—, —C(R^{A1})(R^{A2})—C(R^{A1})(R^{A2})—, and —C(R^{A1})(R^{A2})—O—.

In some embodiments, B is —C(R^{A1})(R^{A2})—.

In some embodiments, B is —C(R^{A1})(R^{A2})—C(R^{A1})(R^{A2})—.

In some embodiments, B is —C(R^{A1})(R^{A2})—O—.

In some embodiments, B is —C(R^{A1})(R^{A2})—, wherein R^{A1} is absent.

In some embodiments, B is —C(R^{A1})(R^{A2})—C(R^{A1})(R^{A2})—, wherein one R^{A1} is absent.

In some embodiments, B is —C(R^{A1})(R^{A2})—O—, wherein R^{A1} is absent.

In some embodiments, B is —C(R^{A2})—.

In some embodiments, B is —C(R^{A2})—C(R^{A1})(R^{A2})—.

In some embodiments, B is —C(R^{A2})—O—.

### Embodiments Covering R^{A1}

In some embodiments, each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH.

In some embodiments, each R^{A1} is independently selected from absent, H, and C₁₋₆ alkyl.

In some embodiments, R^{A1} is absent.

In some embodiments, R^{A1} is H.

In some embodiments, R^{A1} is C₁₋₆ alkyl.

### Embodiments Covering R^{A2}

In some embodiments, each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH.

In some embodiments, each R^{A2} is independently selected from H and C₁₋₆ alkyl.

### Embodiments Covering R^{A3}

In some embodiments, each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH; and
R^{z} is H, C₁₋₆ alkyl, or phenyl.

In some embodiments, each R^{A3} is independently selected from H and C₁₋₆ alkyl.

### Embodiments Covering V

In some embodiments, V is N or CH.

In some embodiments, V is N.

In some embodiments, V is CH.

### Embodiments Covering Y

In some embodiments, Y is N or CH.

In some embodiments, Y is N.

In some embodiments, Y is CH.

### Embodiments Covering G

In some embodiments, G is absent, O, CH₂, or NH, wherein
when G is absent, X is connected with a nitrogen of Ring A;
when G is O, CH₂, or NH, G is connected with the nitrogen of Ring A.

In some embodiments, G is absent, O, CH₂, or NH.

In some embodiments, G is absent and X is connected with a nitrogen of Ring A.

In some embodiments, G is O and G is connected with the nitrogen of Ring A.

In some embodiments, G is CH₂ and G is connected with the nitrogen of Ring A.

In some embodiments, G is NH and G is connected with the nitrogen of Ring A.

### Embodiments Covering Ring A

In some embodiments, Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′); and
each R^{4a}′ and R^{4b}′ is independently H, C₁₋₆ alkyl, C₃₋₁₂ cycloalkyl, or 3- to 12-membered heterocycloalkyl.

In some embodiments, Ring A is 4-18 membered heterocycloalkyl having at least one nitrogen and optionally one or more other heteroatom ring members, wherein the 4-18 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-17 membered heterocycloalkyl having at least one nitrogen and optionally one or more other heteroatom ring members, wherein the 4-17 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-16 membered heterocycloalkyl having at least one nitrogen and optionally one or more other heteroatom ring members, wherein the 4-16 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-15 membered heterocycloalkyl having at least one nitrogen and optionally one or more other heteroatom ring members, wherein the 4-15 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-14 membered heterocycloalkyl having at least one nitrogen and optionally one or more other heteroatom ring members, wherein the 4-14 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-13 membered heterocycloalkyl having at least one nitrogen and optionally one or more other heteroatom ring members, wherein the 4-13 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-12 membered heterocycloalkyl having at least one nitrogen and optionally one or more other heteroatom ring members, wherein the 4-12 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-11 membered heterocycloalkyl having at least one nitrogen and optionally one or more other heteroatom ring members, wherein the 4-11 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-10 membered heterocycloalkyl having at least one nitrogen and optionally one or more other heteroatom ring members, wherein the 4-10 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-9 membered heterocycloalkyl having at least one nitrogen and optionally one or more other heteroatom ring members, wherein the 4-9 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-8 membered heterocycloalkyl having at least one nitrogen and optionally one or more other heteroatom ring members, wherein the 4-8 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-7 membered heterocycloalkyl having at least one nitrogen and optionally one or more other heteroatom ring members, wherein the 4-7 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-6 membered heterocycloalkyl having at least one nitrogen and optionally one or more other heteroatom ring members, wherein the 4-6 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-5 membered heterocycloalkyl having at least one nitrogen and optionally one or more other heteroatom ring members, wherein the 4-5 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 4-membered heterocycloalkyl having at least one nitrogen and optionally one or more other heteroatom ring members, wherein the 4-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 5-membered heterocycloalkyl having at least one nitrogen and optionally one or more other heteroatom ring members, wherein the 5-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 6-membered heterocycloalkyl having at least one nitrogen and optionally one or more other heteroatom ring members, wherein the 6-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 7-membered heterocycloalkyl having at least one nitrogen and optionally one or more other heteroatom ring members, wherein the 7-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 8-membered heterocycloalkyl having at least one nitrogen and optionally one or more other heteroatom ring members, wherein the 8-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 9-membered heterocycloalkyl having at least one nitrogen and optionally one or more other heteroatom ring members, wherein the 9-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 10-membered heterocycloalkyl having at least one nitrogen and optionally one or more other heteroatom ring members, wherein the 10-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 11-membered heterocycloalkyl having at least one nitrogen and optionally one or more other heteroatom ring members, wherein the 11-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 12-membered heterocycloalkyl having at least one nitrogen and optionally one or more other heteroatom ring members, wherein the 12-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 13-membered heterocycloalkyl having at least one nitrogen and optionally one or more other heteroatom ring members, wherein the 13-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 14-membered heterocycloalkyl having at least one nitrogen and optionally one or more other heteroatom ring members, wherein the 14-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 15-membered heterocycloalkyl having at least one nitrogen and optionally one or more other heteroatom ring members, wherein the 15-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 16-membered heterocycloalkyl having at least one nitrogen and optionally one or more other heteroatom ring members, wherein the 16-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 17-membered heterocycloalkyl having at least one nitrogen and optionally one or more other heteroatom ring members, wherein the 17-membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 18-membered heterocycloalkyl having at least one nitrogen and optionally one or more other heteroatom ring members, wherein the 18-membered heterocycloalkyl is optionally substituted with one or more R³;

each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is

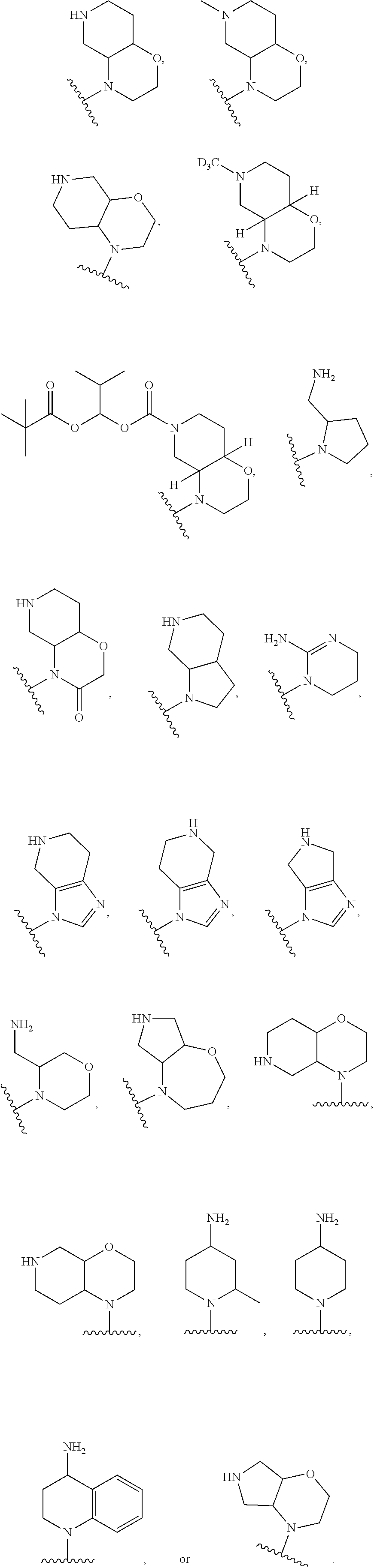

In some embodiments, Ring A is

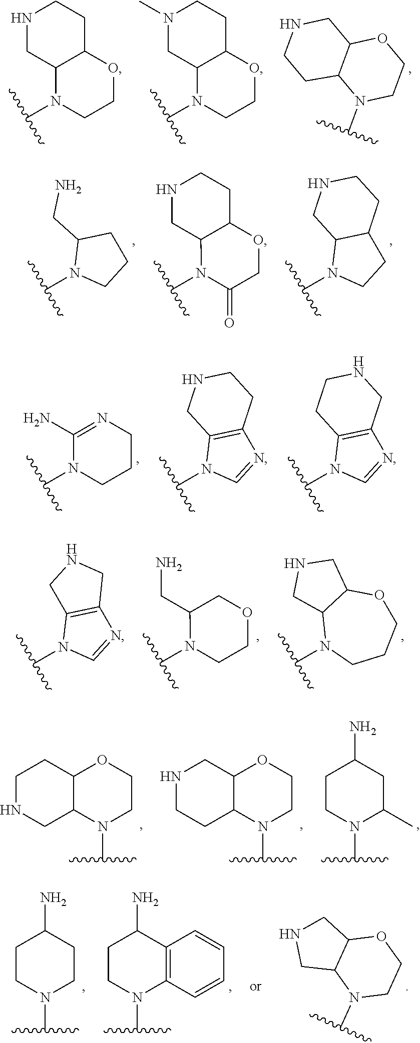

In some embodiments, Ring A is

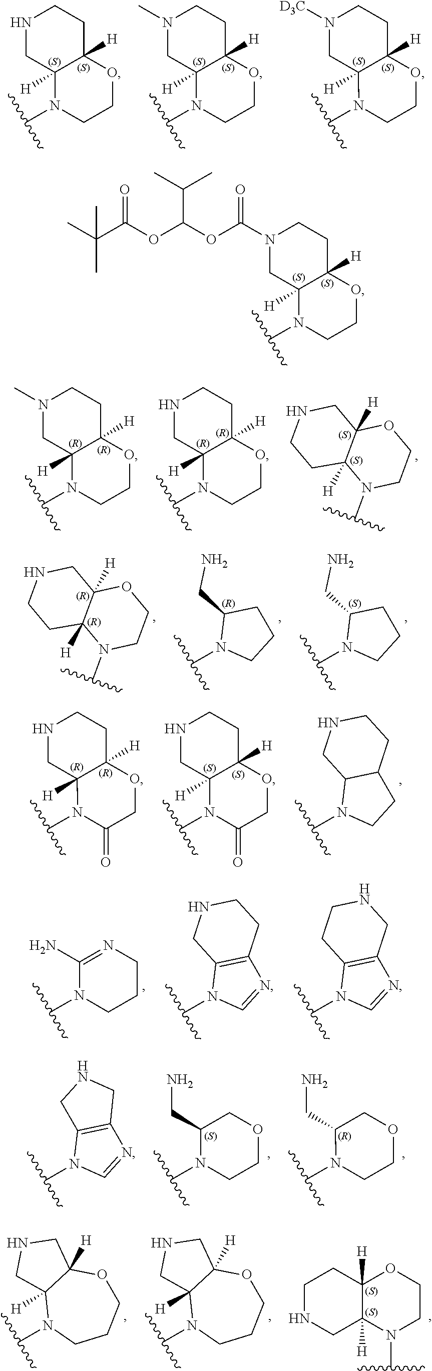

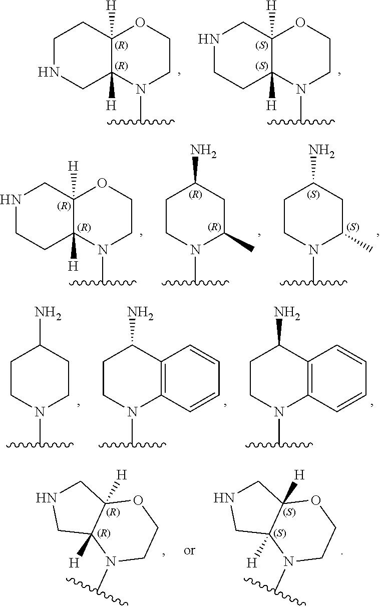

In some embodiments, Ring A is

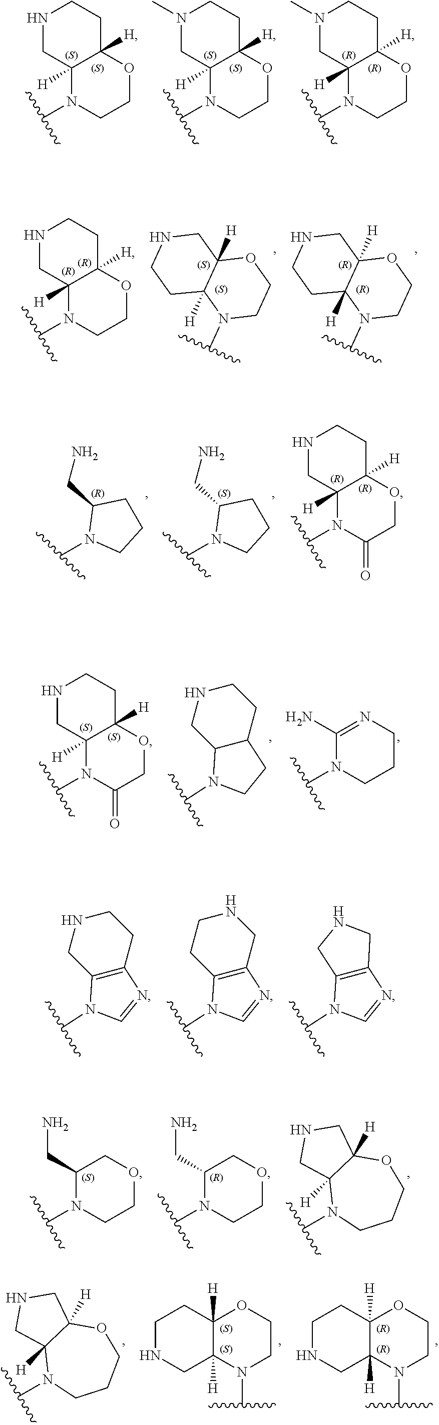

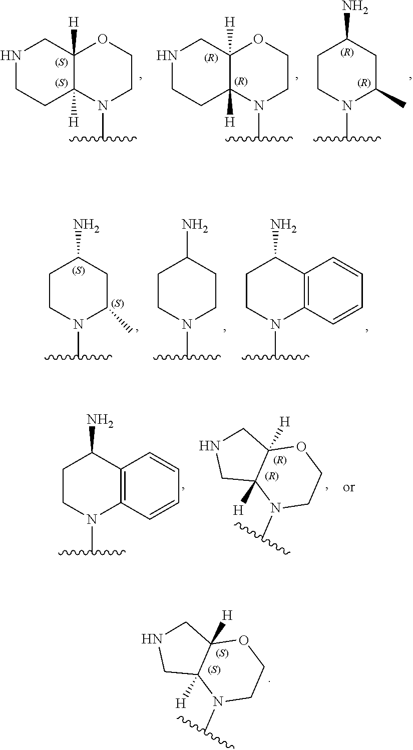

In some embodiments, Ring A is 5- to 10-membered heteroaryl having at least one nitrogen and optionally one or more other heteroatom ring members, wherein the 5- to 10-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 5- to 9-membered heteroaryl having at least one nitrogen and optionally one or more other heteroatom ring members, wherein the 5- to 9-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 5- to 8-membered heteroaryl having at least one nitrogen and optionally one or more other heteroatom ring members, wherein the 5- to 8-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 5- to 7-membered heteroaryl having at least one nitrogen and optionally one or more other heteroatom ring members, wherein the 5- to 7-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 5- to 6-membered heteroaryl having at least one nitrogen and optionally one or more other heteroatom ring members, wherein the 5- to 6-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 5-membered heteroaryl having at least one nitrogen and optionally one or more other heteroatom ring members, wherein the 5-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 6-membered heteroaryl having at least one nitrogen and optionally one or more other heteroatom ring members, wherein the 6-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 7-membered heteroaryl having at least one nitrogen and optionally one or more other heteroatom ring members, wherein the 7-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 8-membered heteroaryl having at least one nitrogen and optionally one or more other heteroatom ring members, wherein the 8-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is 9-membered heteroaryl having at least one nitrogen and optionally one or more other heteroatom ring members, wherein the 9-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

In some embodiments, Ring A is

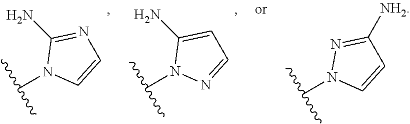 [SMILES: *n1ccnc1N ; *n1nccc1N ; *n1ccc(N)n1]

### Embodiments Covering W

In some embodiments, W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino.

In some embodiments, W is CR^{W}, wherein R^{W} is H, halo, CN, OH, C₁₋₆ alkyl, C₁₋₆ alkoxy, amino, C₁₋₆ alkyl amino, or C₂₋₈ dialkylamino.

In some embodiments, W is N.

In some embodiments, W is CH.

### Embodiments Covering X

In some embodiments, X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino.

In some embodiments, X is N.

In some embodiments, X is CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino.

In some embodiments, X is CH.

In some embodiments, X is C.

In some embodiments, when [symbol] is a double bond, X is C.

### Embodiments Covering the Spiro Moiety

In some embodiments, the spiro moiety represented by the below formula:

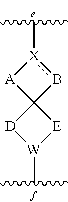

wherein e and f indicate points of attachment to the remainder of the molecule, is selected from:

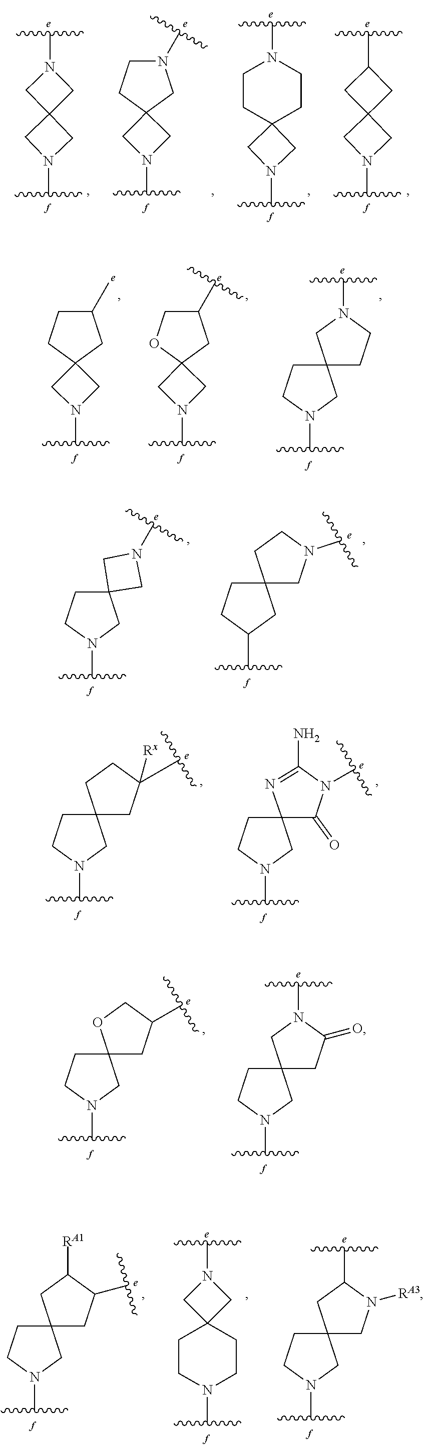

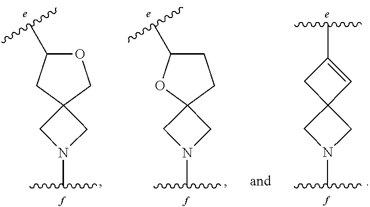 [SMILES: *C1CC2(CO1)CN(*)C2 ; *C1CCC2(CN(*)C2)O1 ; *C1=CC2(C1)CN(*)C2]

In some embodiments, the spiro moiety represented by the below formula:

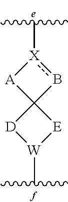

wherein e and f indicate points of attachment to the remainder of the molecule, is selected from:

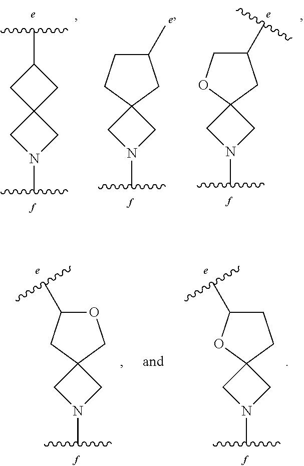

### Embodiments Covering Z

In some embodiments, Z is Cy², halo, C₁₋₆ alkyl, C₁₋₆ haloalkyl, C₁₋₆ cyanoalkyl, C₂₋₆ alkenyl, C₂₋₆ alkynyl, CN, NO₂, OR^{a3}, SR^{a3}, C(O)R^{b3}, C(O)NR¹R², C(O)OR^{a3}, OC(O)R^{b3}OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3} NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3}, S(O)R^{b3} S(O)NR^{c3}R^{d3}, S(O)₂R^{b3}, S(O)₂NR^{c3}R^{d3}, and P(O)R^{c3}R^{d3} wherein said C₁₋₆ alkyl, C₁₋₆ haloalkyl, C₁₋₆ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}C(O)NR^{c3}R^{d3}, C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3}, NR^{c3}C(O)OR^{a3}, NR^{c3}C(O)NR^{c3}R^{d3} NR^{c3}S(O)R^{b3} NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R⁵, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)RVs, S(O)NR^{c5}R^{d5}, S(O)₂R^{b5}, and S(O)₂NR^{c5}R^{d5}, wherein said C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a}s OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{a3}, R^{b3}, R^{c3}, R^{d3}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e3} and R^{e}s is independently selected from H, C₁₋₆ alkyl, and CN;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein R¹ and R² are as defined above.

In some embodiments, Z is C(O)NR¹R², wherein:
R¹ is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein:
R¹ is 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein:
R¹ is —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}), N(R^{3a}′)(R^{3b}′) or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein:
R¹ is 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′,
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein:
R¹ is C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, Z is C(O)NR¹R², wherein R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy.

### Embodiments Covering R¹

In some embodiments, R¹ is:
(a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
(c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a} is independently H, CN, or C₁₋₆ alkyl optionally substituted with one or more halo, C₆₋₁₀ aryl, or N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, R¹ is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, R¹ is C₁₋₆ alkyl.

In some embodiments, R¹ is C₁₋₅ alkyl.

In some embodiments, R¹ is C₁₋₆ alkyl.

In some embodiments, R¹ is C₁₋₃ alkyl.

In some embodiments, R¹ is C₁₋₂ alkyl.

In some embodiments, R¹ is methyl.

In some embodiments, R¹ is ethyl.

In some embodiments, R¹ is propyl.

In some embodiments, R¹ is isopropyl.

In some embodiments, R¹ is butyl.

In some embodiments, R¹ is tert-butyl.

In some embodiments, R¹ is pentyl.

In some embodiments, R¹ is hexyl.

In some embodiments, R¹ is C₁₋₆ haloalkyl.

In some embodiments, R¹ is C₁₋₃ haloalkyl.

In some embodiments, R¹ is C₁₋₂haloalkyl.

In some embodiments, R¹ is —CH₂—CF₃, —CH₂—CHF₂, or —CH₂—CH₂F.

In some embodiments, R¹ is —CH₂—CHF₂.

In some embodiments, R¹ is C₁₋₆ alkoxy.

In some embodiments, R¹ is C₂₋₆ alkenyl.

In some embodiments, R¹ is C₂₋₆ alkynyl.

In some embodiments, R¹ is C₆₋₁₀ aryl.

In some embodiments, R¹ is C₃₋₁₂ cycloalkyl.

In some embodiments, R¹ is 5-10 membered heteroaryl.

In some embodiments, R¹ is 3-12 membered heterocycloalkyl.

In some embodiments, R¹ is —C₆₋₁₀ aryl-C₁₋₆ alkyl.

In some embodiments, R¹ is —C₃₋₁₂ cycloalkyl-C₁₋₆ alkyl.

In some embodiments, R¹ is -(5-10 membered heteroaryl)-C₁₋₆ alkyl.

In some embodiments, R¹ is -(4-10 membered heterocycloalkyl)-C₁₋₆ alkyl.

In some embodiments, R¹ is 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
each R^{4a} is independently H, CN, or C₁₋₆ alkyl optionally substituted with one or more halo, C₆₋₁₀ aryl, or N(R^{4a}′)(R^{4b}′); and
each R^{4a}′ and R^{4b}′ is independently H, C₁₋₆ alkyl, C₃₋₁₂ cycloalkyl, or 3- to 12-membered heterocycloalkyl.

In some embodiments, R¹ is —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₁₁ cycloalkyl substituted with one or more 3- to 12-membered heterocycloalkyl which has at least one nitrogen and optionally one or more other heteroatom ring members; and wherein each of the C₃₋₁₁ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₁₀ cycloalkyl substituted with one or more 3- to 12-membered heterocycloalkyl which has at least one nitrogen and optionally one or more other heteroatom ring members; and wherein each of the C₃₋₁₀ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₉ cycloalkyl substituted with one or more 3- to 12-membered heterocycloalkyl which has at least one nitrogen and optionally one or more other heteroatom ring members; and wherein each of the C₃₋₉ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₈ cycloalkyl substituted with one or more 3- to 12-membered heterocycloalkyl which has at least one nitrogen and optionally one or more other heteroatom ring members; and wherein each of the C₃₋₈ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₇ cycloalkyl substituted with one or more 3- to 12-membered heterocycloalkyl which has at least one nitrogen and optionally one or more other heteroatom ring members; and wherein each of the C₃₋₇ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₆ cycloalkyl substituted with one or more 3- to 12-membered heterocycloalkyl which has at least one nitrogen and optionally one or more other heteroatom ring members; and wherein each of the C₃₋₆ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₅ cycloalkyl substituted with one or more 3- to 12-membered heterocycloalkyl which has at least one nitrogen and optionally one or more other heteroatom ring members; and wherein each of the C₃₋₅ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is C₃₋₄ cycloalkyl substituted with one or more 3- to 12-membered heterocycloalkyl which has at least one nitrogen and optionally one or more other heteroatom ring members; and wherein each of the C₃₋₄ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclopropyl substituted with one or more 3- to 12-membered heterocycloalkyl which has at least one nitrogen and optionally one or more other heteroatom ring members; and wherein each of the cyclopropyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclobutyl substituted with one or more 3- to 12-membered heterocycloalkyl which has at least one nitrogen and optionally one or more other heteroatom ring members; and wherein each of the cyclobutyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclopentyl substituted with one or more 3- to 12-membered heterocycloalkyl which has at least one nitrogen and optionally one or more other heteroatom ring members; and wherein each of the cyclopentyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclohexyl substituted with one or more 3- to 12-membered heterocycloalkyl which has at least one nitrogen and optionally one or more other heteroatom ring members; and wherein each of the cyclohexyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cycloheptyl substituted with one or more 3- to 12-membered heterocycloalkyl which has at least one nitrogen and optionally one or more other heteroatom ring members; and wherein each of the cycloheptyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclooctyl substituted with one or more 3- to 12-membered heterocycloalkyl which has at least one nitrogen and optionally one or more other heteroatom ring members; and wherein each of the cyclooctyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclononyl substituted with one or more 3- to 12-membered heterocycloalkyl which has at least one nitrogen and optionally one or more other heteroatom ring members; and wherein each of the cyclononyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is cyclodecyl substituted with one or more 3- to 12-membered heterocycloalkyl which has at least one nitrogen and optionally one or more other heteroatom ring members; and wherein each of the cyclodecyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

In some embodiments, R¹ is

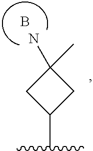

wherein Ring B is a 3- to 12-membered heterocycloalkyl which has at least one nitrogen and optionally one or more other heteroatom ring members, wherein the heterocycloalkyl is optionally substituted.

In some embodiments, R¹ is

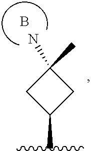

wherein Ring B is a 3- to 12-membered heterocycloalkyl which has at least one nitrogen and optionally one or more other heteroatom ring members, wherein the heterocycloalkyl is optionally substituted, wherein R^{Bn} is an optionally substituted C₁₋₆ alkyl.

In some embodiments, R¹ is

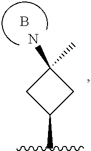

wherein Ring B is a 3- to 12-membered heterocycloalkyl which has at least one nitrogen and optionally one or more other heteroatom ring members, wherein the heterocycloalkyl is optionally substituted, wherein R^{Bn} is an optionally substituted C₁₋₆ alkyl.

In some embodiments, R¹ is

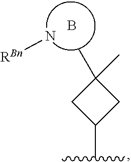

wherein Ring B is a 3- to 12-membered heterocycloalkyl which has at least one nitrogen and optionally one or more other heteroatom ring members, wherein the heterocycloalkyl is optionally substituted, wherein R^{Bn} is an optionally substituted C₁₋₆ alkyl.

In some embodiments, R¹ is

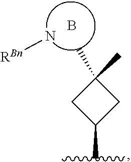

wherein Ring B is a 3- to 12-membered heterocycloalkyl which has at least one nitrogen and optionally one or more other heteroatom ring members, wherein the heterocycloalkyl is optionally substituted, wherein R^{Bn} is an optionally substituted C₁₋₆ alkyl.

In some embodiments, R¹ is

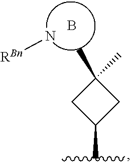

wherein Ring B is a 3- to 12-membered heterocycloalkyl which has at least one nitrogen and optionally one or more other heteroatom ring members, wherein the heterocycloalkyl is optionally substituted, wherein R^{Bn} is an optionally substituted C₁₋₆ alkyl.

In some embodiments, R^{Bn} is an optionally substituted C₁ alkyl.

In some embodiments, R^{Bn} is an optionally substituted C₂ alkyl.

In some embodiments, R^{Bn} is an optionally substituted C₃ alkyl.

In some embodiments, R^{Bn} is a C₁ alkyl.

In some embodiments, R^{B}n is a C₂ alkyl.

In some embodiments, R^{B}n is a C₃ alkyl.

In some embodiments, R¹ is

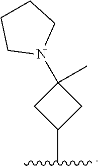 [SMILES: *C1CC(C)(N2CCCC2)C1]

In some embodiments, R¹ is

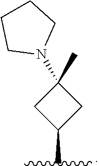 [SMILES: *[C@H]1C[C@](C)(N2CCCC2)C1]

In some embodiments, R¹ is

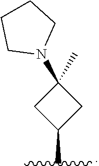 [SMILES: *[C@H]1C[C@@](C)(N2CCCC2)C1]

In some embodiments, R¹ is

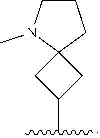 [SMILES: *C1CC2(CCCN2C)C1]

In some embodiments, R¹ is

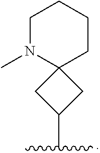 [SMILES: *C1CC2(CCCCN2C)C1]

In some embodiments, R¹ is

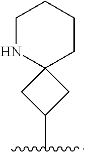 [SMILES: *C1CC2(CCCCN2)C1]

In some embodiments, R¹ is

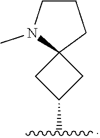 [SMILES: *[C@H]1C[C@@]2(CCCN2C)C1]

In some embodiments, R¹ is

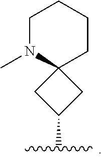 [SMILES: *[C@H]1C[C@@]2(CCCCN2C)C1]

In some embodiments, R¹ is

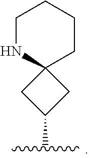 [SMILES: *[C@H]1C[C@@]2(CCCCN2)C1]

### Embodiments Covering R²

In some embodiments, R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl.

In some embodiments, R² is H.

In some embodiments, R² is C₁₋₆ alkyl.

In some embodiments, R² is C₁₋₅ alkyl.

In some embodiments, R² is C₁₋₆ alkyl.

In some embodiments, R² is C₁₋₃ alkyl.

In some embodiments, R² is C₁₋₂ alkyl.

In some embodiments, R² is methyl.

In some embodiments, R² is ethyl.

In some embodiments, R² is propyl.

In some embodiments, R² is isopropyl.

In some embodiments, R² is butyl.

In some embodiments, R² is tert-butyl.

In some embodiments, R² is pentyl.

In some embodiments, R² is hexyl.

### Embodiments Covering R¹ and R²

In some embodiments, R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 3- to 11-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 3- to 10-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 3- to 9-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 3- to 8-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 3- to 7-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 3- to 6-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 3- to 5-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 3- to 4-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 3-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 4-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 5-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 6-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² form a 6-membered heterocycloalkyl with the nitrogen to which they are connected, the heterocycloalkyl is optionally substituted with one or more C₁₋₆ alkyl, halo, OH, CN, or C₁₋₆ alkoxy.

In some embodiments, R¹ and R² form

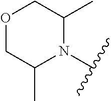 [SMILES: *N1C(C)COCC1C]

group with the nitrogen to which they are connected.

In some embodiments, R¹ and R² form

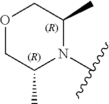

group with the nitrogen to which they are connected.

In some embodiments, R¹ and R² optionally form a 7-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 8-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 9-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 10-membered heterocycloalkyl with the nitrogen to which they are connected, wherein the heterocycloalkyl is optionally substituted with one or more C₁₋₆ alkyl, halo, OH, CN, or C₁₋₆ alkoxy.

In some embodiments, R¹ and R² optionally form a 11-membered heterocycloalkyl with the nitrogen to which they are connected, wherein the heterocycloalkyl is optionally substituted with one or more C₁₋₆ alkyl, halo, OH, CN, or C₁₋₆ alkoxy.

### Embodiments Covering R³

In some embodiments, each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′); and
each R^{4a}′ and R^{4b}′ is independently H, C₁₋₆ alkyl, C₃₋₁₂ cycloalkyl, or 3- to 12-membered heterocycloalkyl.

In some embodiments, R³ is H.

In some embodiments, R³ is oxo.

In some embodiments, R³ is N(R^{4a})(R^{4b}), wherein:
R^{4a} is H, CN, or C₁₋₆ alkyl optionally substituted with one or more halo, C₆₋₁₀ aryl, or N(R^{4a}′)(R^{4b}′);
R^{4b} is H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′); and
each R^{4a}′ and R^{4b}′ is independently H, C₁₋₆ alkyl, C₃₋₁₂ cycloalkyl, or 3- to 12-membered heterocycloalkyl.

In some embodiments, R³ is —NH₂.

In some embodiments, R³ is C₁₋₆ alkyl, wherein the C₁₋₆ alkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′); and
each R^{4a}′ and R^{4b}′ is independently H, C₁₋₆ alkyl, C₃₋₁₂ cycloalkyl, or 3- to 12-membered heterocycloalkyl.

In some embodiments, R³ is C₁₋₆ alkyl substituted with one N(R^{4a})(R^{4b}), wherein:
R^{4a} is H or C₁₋₆ alkyl; and
R^{4b} is H or C₁₋₆ alkyl.

In some embodiments, R³ is —CH₂—NH₂.

In some embodiments, R³ is C₁₋₆ alkyl.

In some embodiments, R³ is C₁₋₅ alkyl.

In some embodiments, R³ is C₁₋₆ alkyl.

In some embodiments, R³ is C₁₋₃ alkyl.

In some embodiments, R³ is C₁₋₂ alkyl.

In some embodiments, R³ is methyl.

In some embodiments, R³ is ethyl.

In some embodiments, R³ is propyl.

In some embodiments, R³ is isopropyl.

In some embodiments, R³ is butyl.

In some embodiments, R³ is tert-butyl.

In some embodiments, R³ is pentyl.

In some embodiments, R³ is hexyl.

In some embodiments, R³ is C₂₋₆ alkenyl.

In some embodiments, R³ is C₂₋₆ alkynyl.

In some embodiments, R³ is C₁₋₆ alkoxy.

In some embodiments, R³ is C₃₋₁₂ cycloalkyl.

In some embodiments, R³ is C₆₋₁₀ aryl.

In some embodiments, R³ is 5- to 10-membered heteroaryl.

In some embodiments, R³ is 3- to 12-membered heterocycloalkyl.

In some embodiments, R³ is C(O)(R^{4a}), wherein:
R^{4a} is H, CN, or C₁₋₆ alkyl optionally substituted with one or more halo, C₆₋₁₀ aryl, or N(R^{4a}′)(R^{4b}′); and
each R^{4a}′ and R^{4b}′ is independently H, C₁₋₆ alkyl, C₃₋₁₂ cycloalkyl, or 3- to 12-membered heterocycloalkyl.

In some embodiments, R³ is C(O)(OR^{4a}), wherein:
R^{4a} is H, CN, or C₁₋₆ alkyl optionally substituted with one or more halo, C₆₋₁₀ aryl, or N(R^{4a}′)(R^{4b}′); and
each R^{4a}′ and R^{4b}′ is independently H, C₁₋₆ alkyl, C₃₋₁₂ cycloalkyl, or 3- to 12-membered heterocycloalkyl.

In some embodiments, R³ is C(O)O—C(R^{4a})₂—OC(O)(R^{4a}), wherein:
each R^{4a} is independently H, CN, or C₁₋₆ alkyl optionally substituted with one or more halo, C₆₋₁₀ aryl, or N(R^{4a}′)(R^{4b}′); and
each R^{4a}′ and R^{4b}′ is independently H, C₁₋₆ alkyl, C₃₋₁₂ cycloalkyl, or 3- to 12-membered heterocycloalkyl.

### Exemplary Combinations

In some embodiments, V is CH, Y is N, and G is CH₂ and G is connected with the nitrogen of Ring A.

In some embodiments, V is CH; Y is N; G is CH₂ and G is connected with the nitrogen of Ring A; and Ring A is

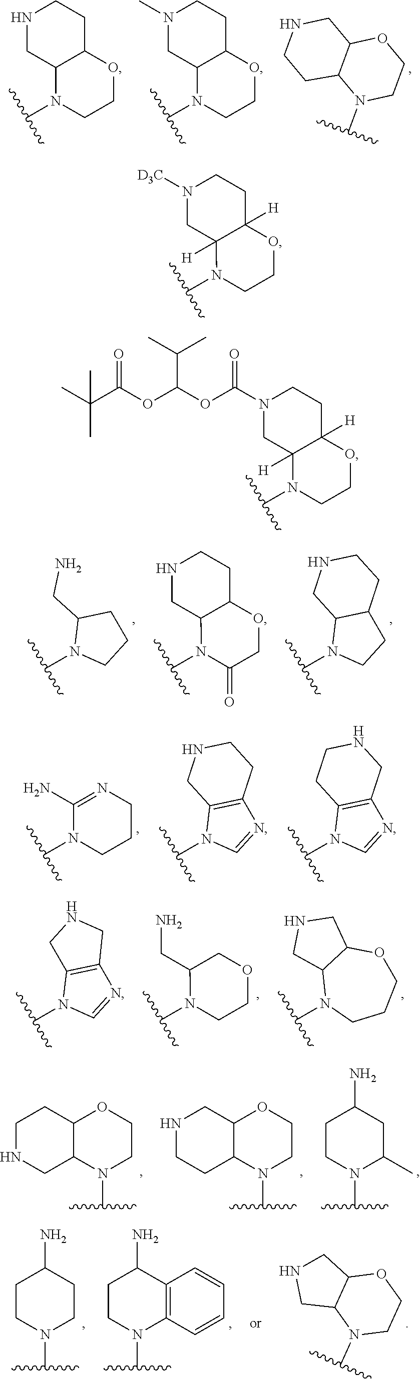

In some embodiments, V is CH; Y is N; G is CH₂ and G is connected with the nitrogen of Ring A; and Ring A is

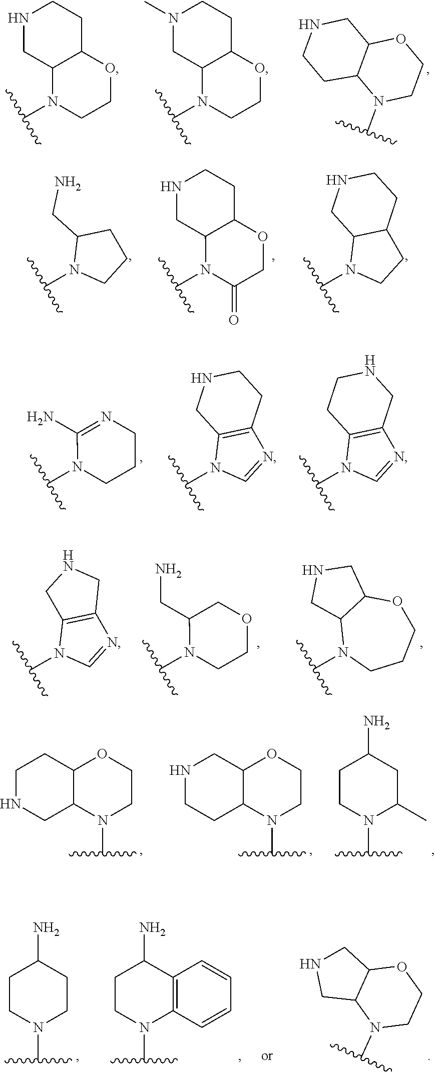

In some embodiments, V is CH; Y is N; G is CH₂ and G is connected with the nitrogen of Ring A; and Ring A is

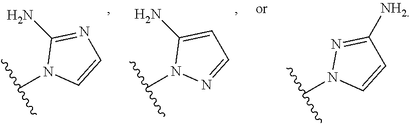 [SMILES: *n1ccnc1N ; *n1nccc1N ; *n1ccc(N)n1]

In some embodiments, V is CH, Y is N, G is CH₂ and G is connected with the nitrogen of Ring A, and R² is isopropyl.

In some embodiments, V is CH; Y is N; G is CH₂ and G is connected with the nitrogen of Ring A; and Ring A is

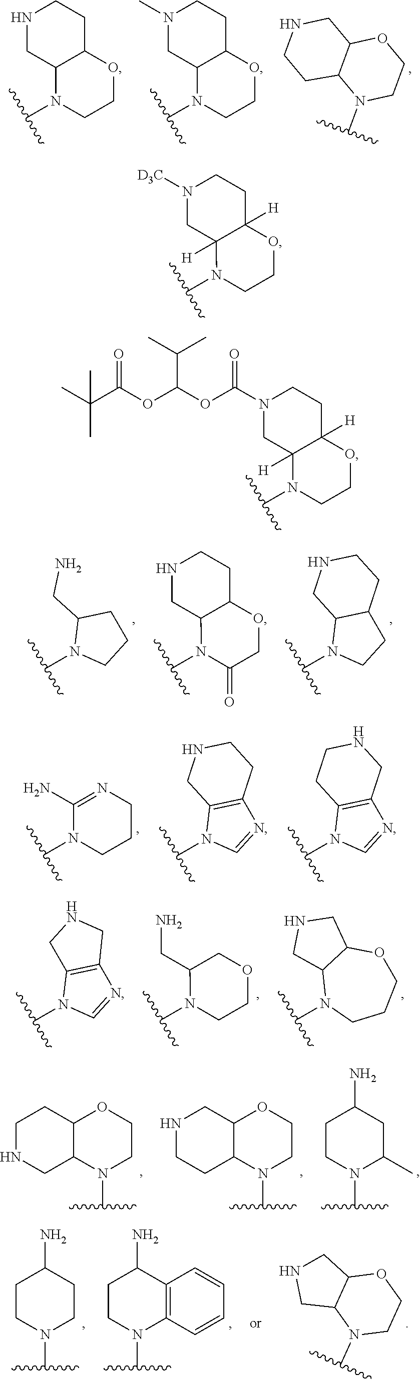

In some embodiments, V is CH; Y is N; G is CH₂ and G is connected with the nitrogen of Ring A; and Ring A is

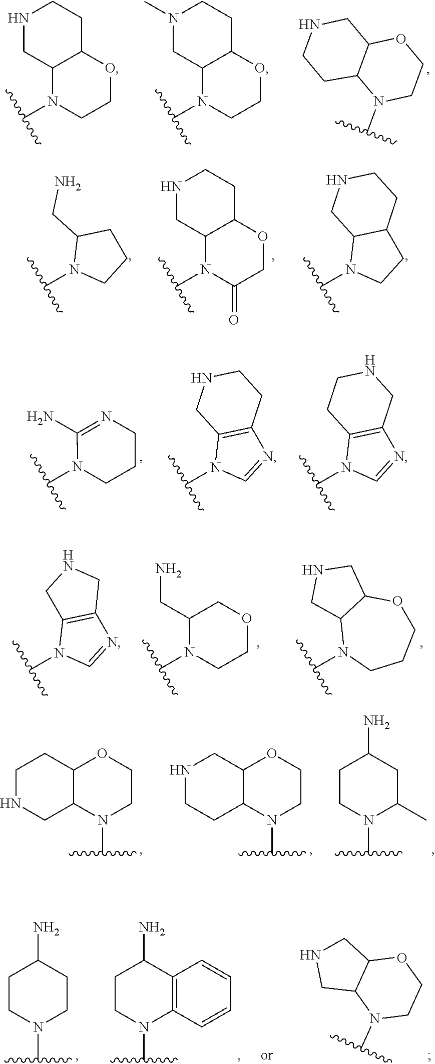

and R² is isopropyl.

In some embodiments, V is CH; Y is N; G is CH₂ and G is connected with the nitrogen of Ring A; Ring A is

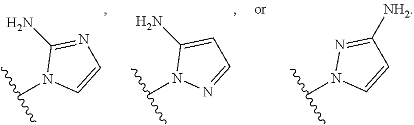 [SMILES: *n1ccnc1N ; *n1nccc1N ; *n1ccc(N)n1]

and R² is isopropyl.

In some embodiments, V is CH; Y is N; G is CH₂ and G is connected with the nitrogen of Ring A; and R¹ is ethyl, isopropyl, —CH₂—CHF₂,

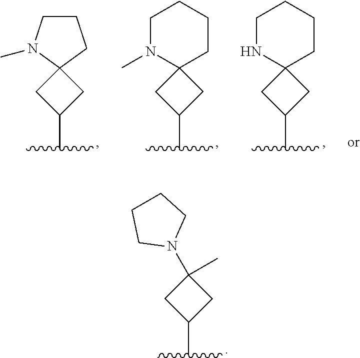

In some embodiments, V is CH; Y is N; G is CH₂ and G is connected with the nitrogen of Ring A; and R¹ is ethyl, isopropyl, —CH₂—CHF₂, or

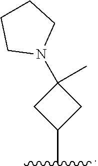 [SMILES: *C1CC(C)(N2CCCC2)C1]

In some embodiments V is CH; Y is N; G is CH₂ and G is connected with the nitrogen of Ring A; Ring A is

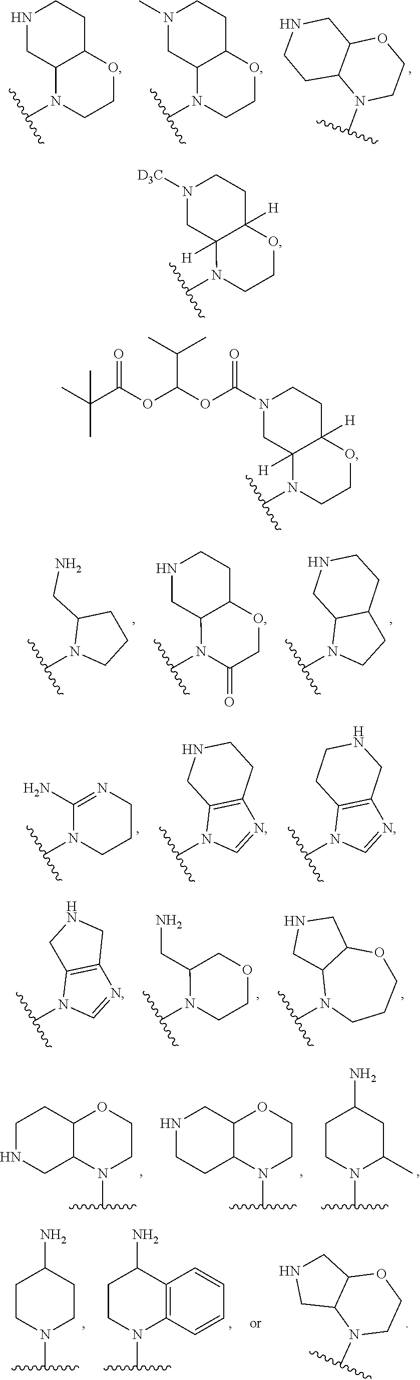

and R¹ is ethyl, isopropyl, —CH₂—CHF₂,

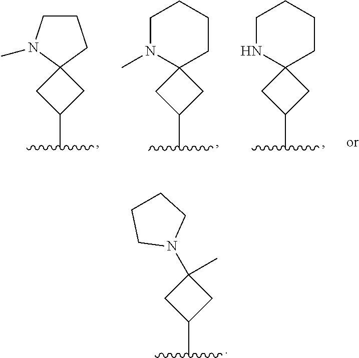

In some embodiments, V is CH; Y is N; G is CH₂ and G is connected with the nitrogen of Ring A; Ring A is

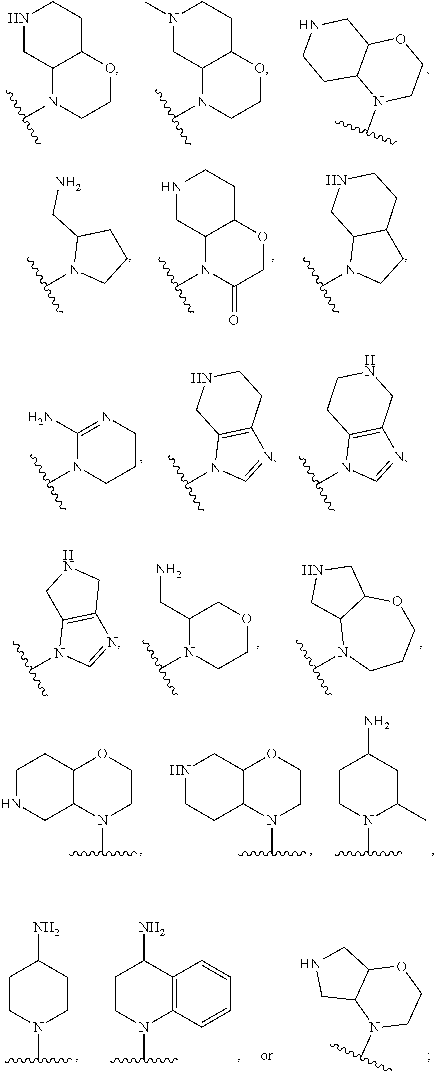

and R¹ is ethyl, isopropyl, —CH₂—CHF₂, or

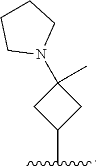 [SMILES: *C1CC(C)(N2CCCC2)C1]

In some embodiments, V is CH; Y is N; G is CH₂ and G is connected with the nitrogen of Ring A; Ring A is

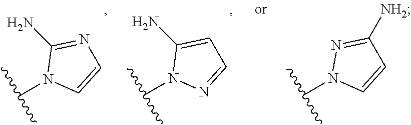 [SMILES: *n1ccnc1N ; *n1nccc1N ; *n1ccc(N)n1]

and R¹ is ethyl, isopropyl, —CH₂—CHF₂,

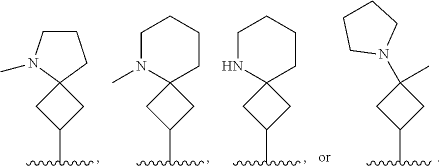

In some embodiments, V is CH; Y is N; G is CH₂ and G is connected with the nitrogen of Ring A; Ring A is

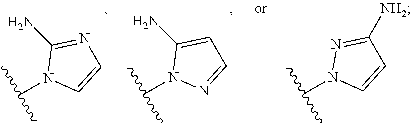 [SMILES: *n1ccnc1N ; *n1nccc1N ; *n1ccc(N)n1]

and R¹ is ethyl, isopropyl, —CH₂—CHF₂, or

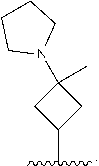 [SMILES: *C1CC(C)(N2CCCC2)C1]

In some embodiments, V is CH, Y is N, G is CH₂ and G is connected with the nitrogen of Ring A; R¹ is ethyl, isopropyl, —CH₂—CHF₂,

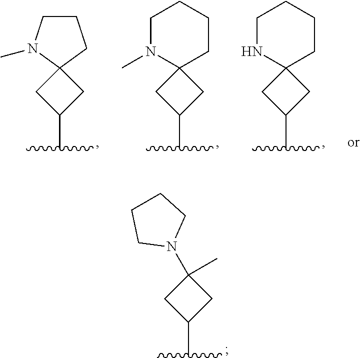

and R² is isopropyl.

In some embodiments, V is CH, Y is N, G is CH₂ and G is connected with the nitrogen of Ring A; R¹ is ethyl, isopropyl, —CH₂—CHF₂,

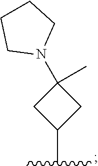 [SMILES: *C1CC(C)(N2CCCC2)C1]

and R² is isopropyl.

In some embodiments, V is CH; Y is N; G is CH₂ and G is connected with the nitrogen of Ring A; Ring A is

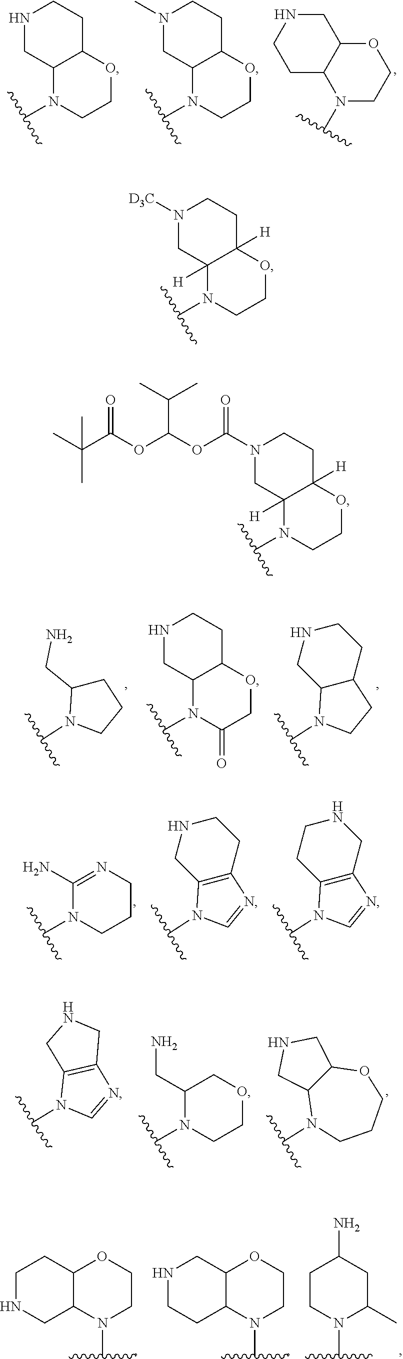

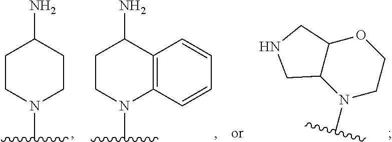

R¹ is ethyl, isopropyl, —CH₂—CHF₂,

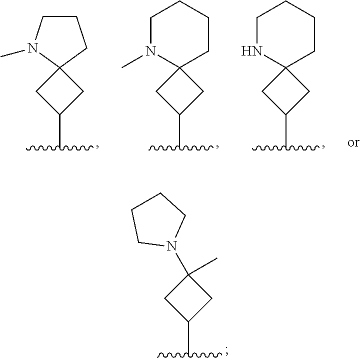

and R² is isopropyl.

In some embodiments, V is CH; Y is N; G is CH₂ and G is connected with the nitrogen of Ring A; Ring A is

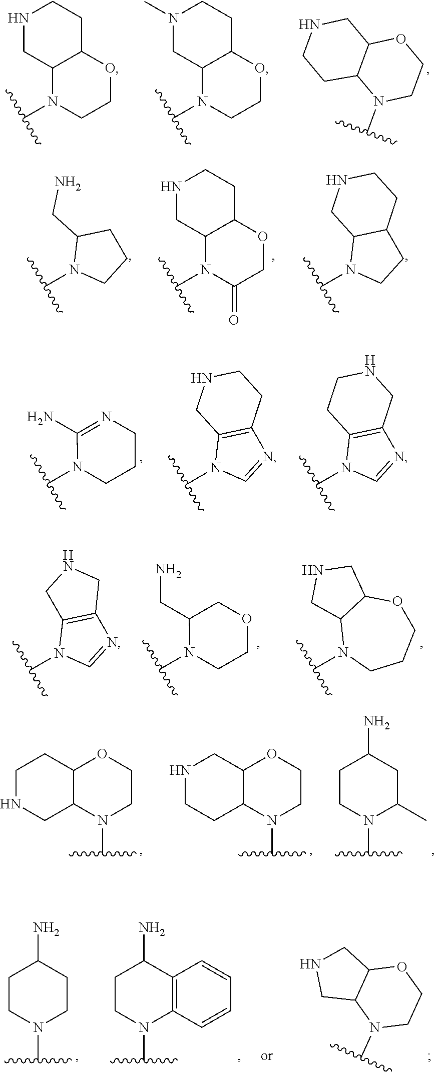

R¹ is ethyl, isopropyl, —CH₂—CHF₂,

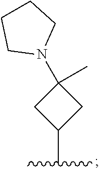 [SMILES: *C1CC(C)(N2CCCC2)C1]

and R² is isopropyl.

In some embodiments, V is CH; Y is N; G is CH₂ and G is connected with the nitrogen of Ring A; Ring A is

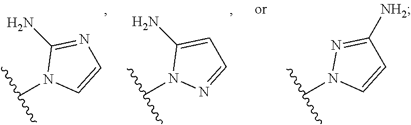 [SMILES: *n1ccnc1N ; *n1nccc1N ; *n1ccc(N)n1]

R¹ is ethyl, isopropyl, —CH₂—CHF₂, or

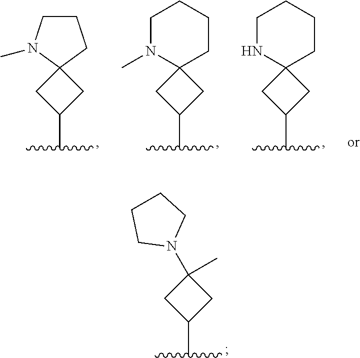

and R² is isopropyl.

In some embodiments, V is CH; Y is N; G is CH₂ and G is connected with the nitrogen of Ring A; Ring A is

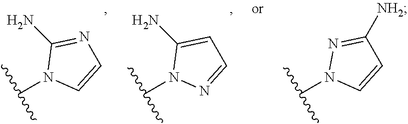 [SMILES: *n1ccnc1N ; *n1nccc1N ; *n1ccc(N)n1]

R¹ is ethyl, isopropyl, —CH₂—CHF₂, or

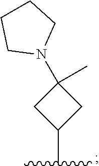 [SMILES: *C1CC(C)(N2CCCC2)C1]

and R² is isopropyl.

In some embodiments, V is CH, Y is N, G is CH₂ and G is connected with the nitrogen of Ring A, and R¹ and R² form

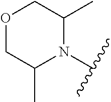 [SMILES: *N1C(C)COCC1C]

group with the nitrogen to which they are connected.

In some embodiments, V is CH; Y is N; G is CH₂ and G is connected with the nitrogen of Ring A; Ring A is

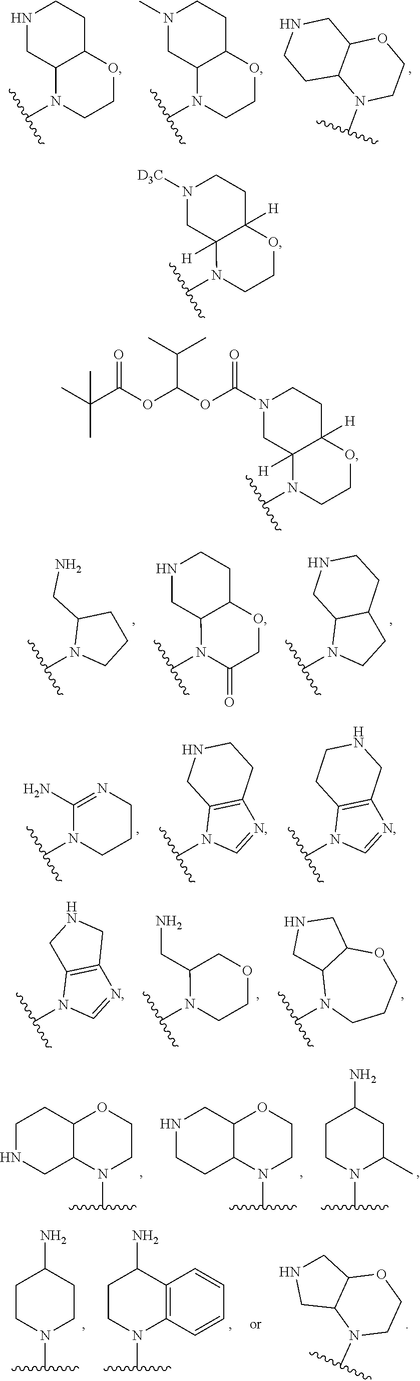

and R¹ and R² form

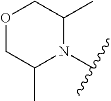 [SMILES: *N1C(C)COCC1C]

group with the nitrogen to which they are connected.

In some embodiments, V is CH; Y is N; G is CH₂ and G is connected with the nitrogen of Ring A; Ring A is

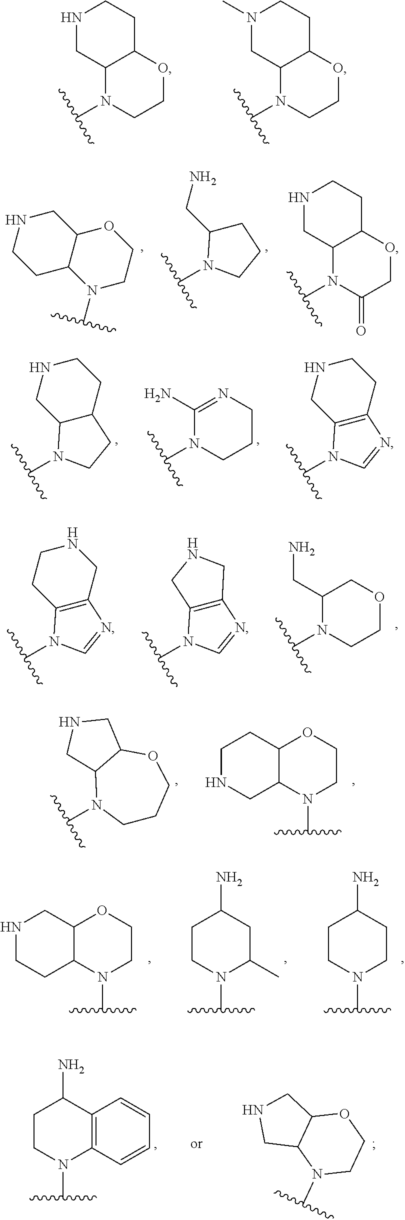

and R¹ and R² form

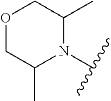 [SMILES: *N1C(C)COCC1C]

group with the nitrogen to which they are connected.

In some embodiments, V is CH; Y is N; G is CH₂ and G is connected with the nitrogen of Ring A; Ring A is

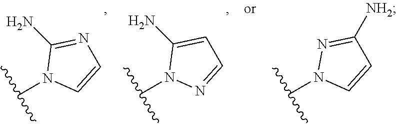 [SMILES: *n1ccnc1N ; *n1nccc1N ; *n1ccc(N)n1]

and R¹ and R² form

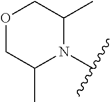 [SMILES: *N1C(C)COCC1C]

group with the nitrogen to which they are connected.

In some embodiments, V is CH, Y is N, G is CH₂ and G is connected with the nitrogen of Ring A; and the spiro moiety represented by the below formula:

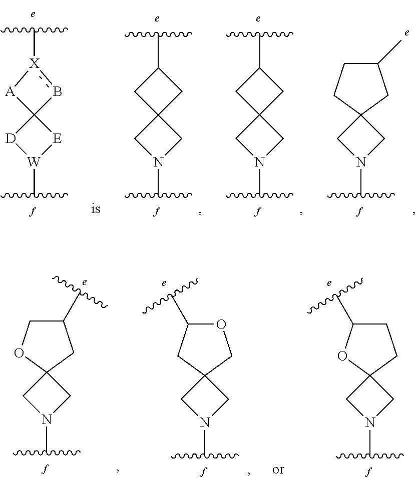

wherein e and f indicate points of attachment to the remainder of the molecule.

In some embodiments, V is CH; Y is N; G is CH₂ and G is connected with the nitrogen of Ring A; Ring A is

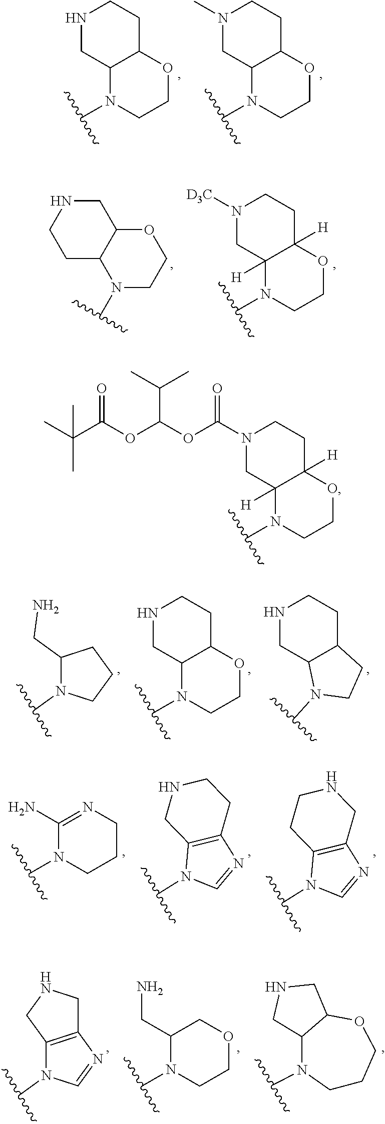

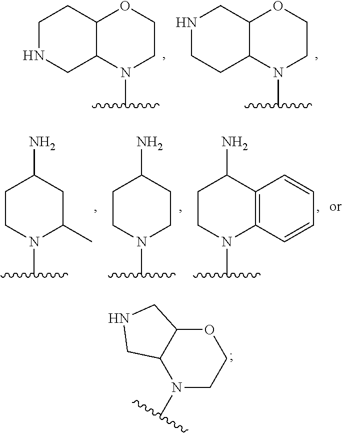

and the spiro moiety represented by the below formula:

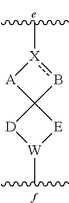

is

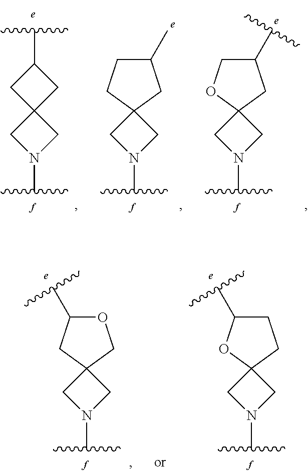

wherein e and f indicate points of attachment to the remainder of the molecule.

In some embodiments, V is CH; Y is N; G is CH₂ and G is connected with the nitrogen of Ring A; Ring A is

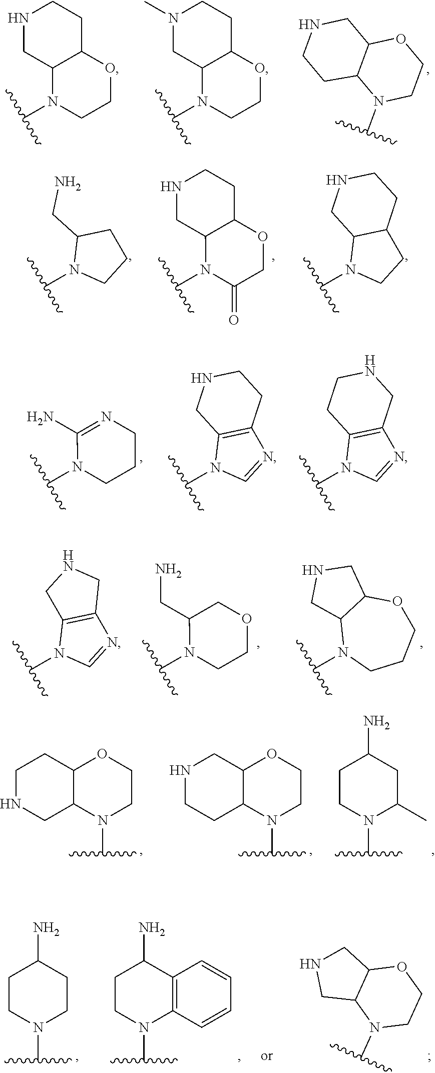

and the spiro moiety represented by the below formula:

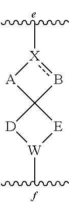

is

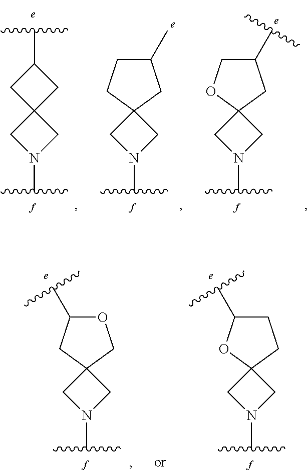

wherein e and f indicate points of attachment to the remainder of the molecule.

In some embodiments, V is CH; Y is N; G is CH₂ and G is connected with the nitrogen of Ring A; Ring A is

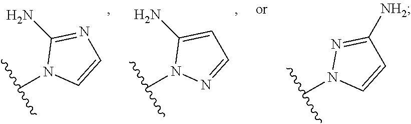 [SMILES: *n1ccnc1N ; *n1nccc1N ; *n1ccc(N)n1]

and the spiro moiety represented by the below formula:

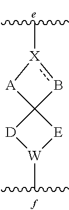

is

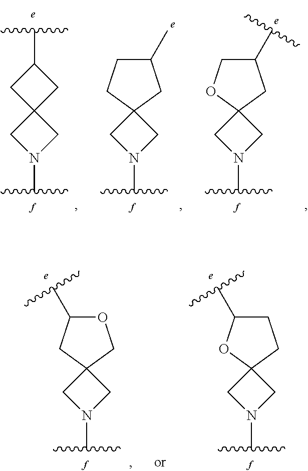

wherein e and f indicate points of attachment to the remainder of the molecule.

In some embodiments, V is CH, Y is N, G is CH₂ and G is connected with the nitrogen of Ring A, R² is isopropyl; and the spiro moiety represented by the below formula:

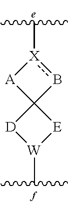

is

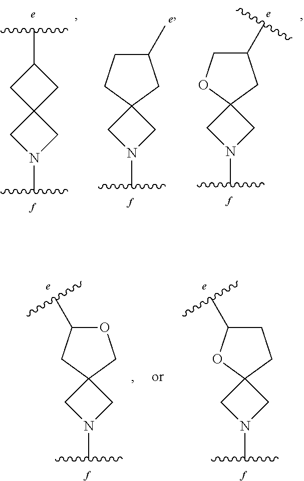

wherein e and f indicate points of attachment to the remainder of the molecule.

In some embodiments, V is CH; Y is N; G is CH₂ and G is connected with the nitrogen of Ring A; Ring A is

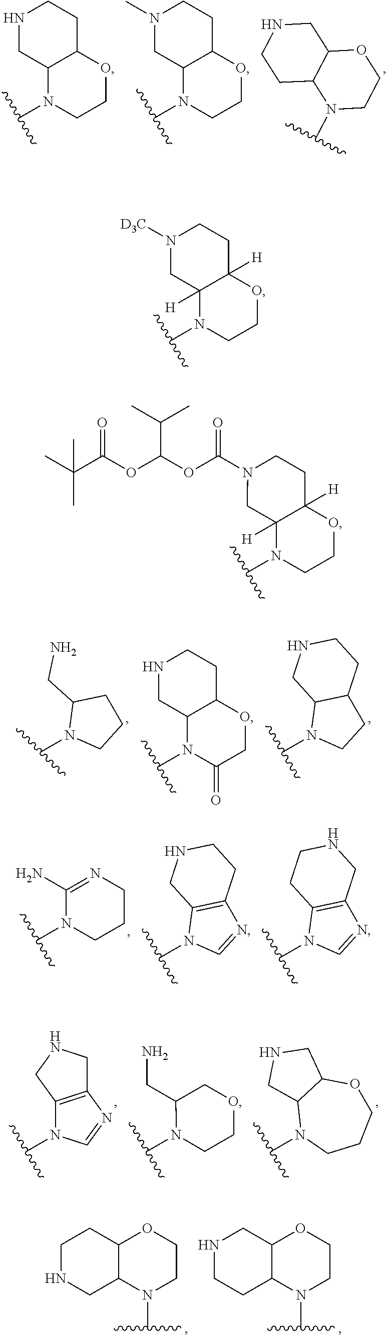

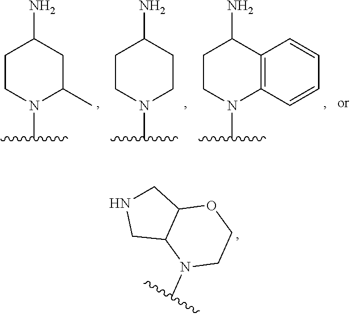

R² is isopropyl; and the spiro moiety represented by the below formula:

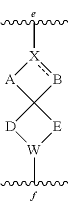

is

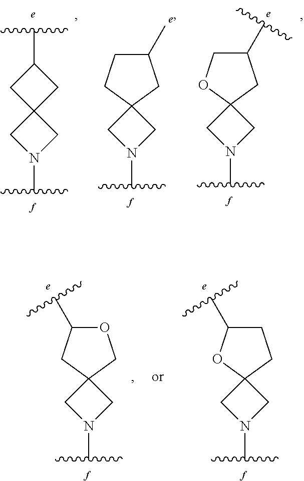

wherein e and f indicate points of attachment to the remainder of the molecule.

In some embodiments, V is CH; Y is N; G is CH₂ and G is connected with the nitrogen of Ring A; Ring A is

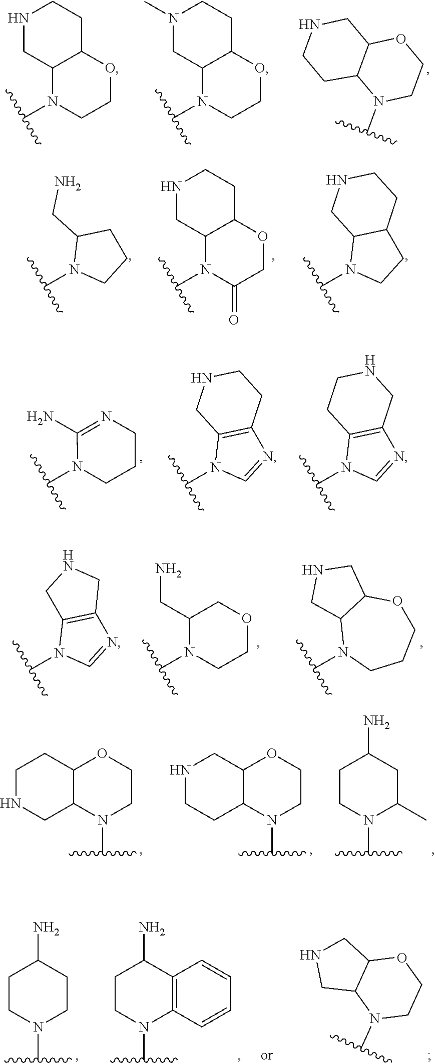

R² is isopropyl; and the spiro moiety represented by the below formula:

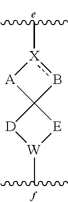

is

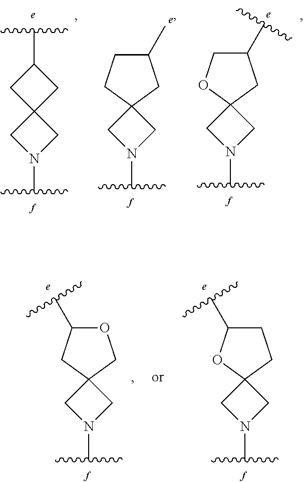

wherein e and f indicate points of attachment to the remainder of the molecule.

In some embodiments V is CH; Y is N; G is CH₂ and G is connected with the nitrogen of Ring A; Ring A is

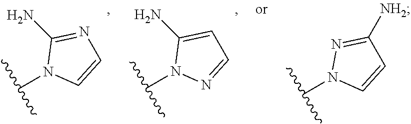 [SMILES: *n1ccnc1N ; *n1nccc1N ; *n1ccc(N)n1]

R² is isopropyl; and the spiro moiety represented by the below formula:

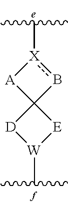

is

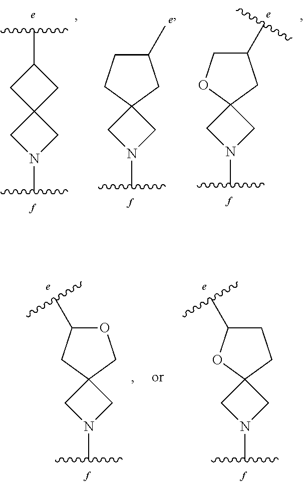

wherein e and f indicate points of attachment to the remainder of the molecule.

In some embodiments, V is CH; Y is N; G is CH₂ and G is connected with the nitrogen of Ring A; R¹ is ethyl, isopropyl, —CH₂—CHF₂,

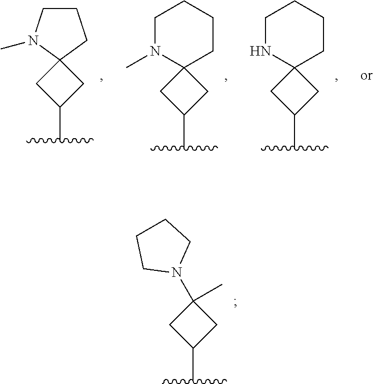

and the spiro moiety represented by the below formula:

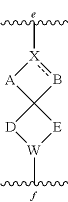

is

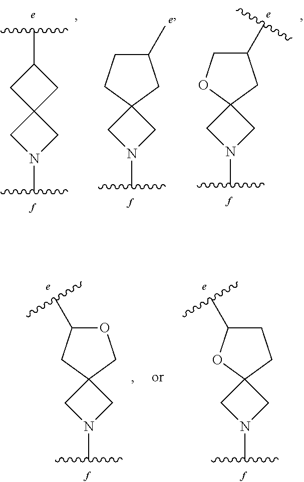

wherein e and f indicate points of attachment to the remainder of the molecule.

In some embodiments, V is CH; Y is N; G is CH₂ and G is connected with the nitrogen of Ring A; R¹ is ethyl, isopropyl, —CH₂—CHF₂,

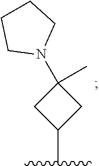 [SMILES: *C1CC(C)(N2CCCC2)C1]

and the spiro moiety represented by the below formula:

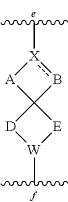

is

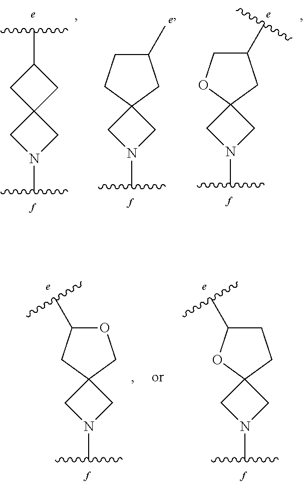

wherein e and f indicate points of attachment to the remainder of the molecule.

In some embodiments, V is CH; Y is N; G is CH₂ and G is connected with the nitrogen of Ring A; Ring A is

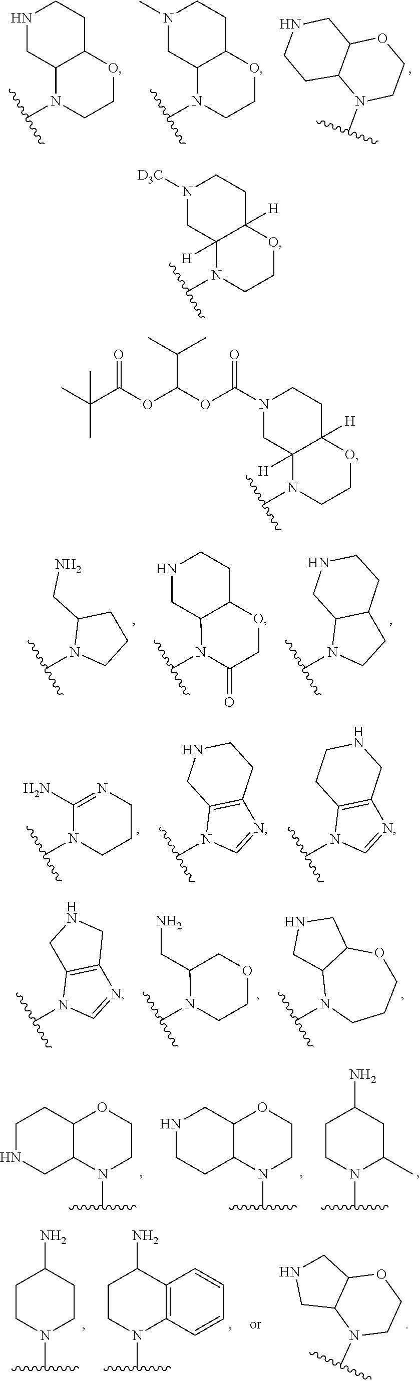

R¹ is ethyl, isopropyl, —CH₂—CHF₂,

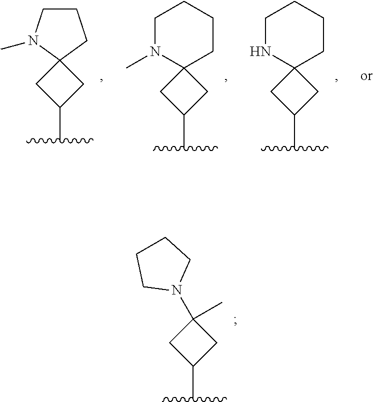

and the spiro moiety represented by the below formula:

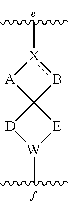

is

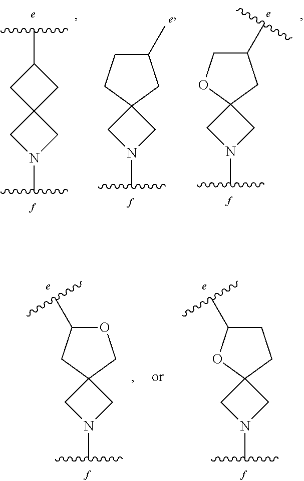

wherein e and f indicate points of attachment to the remainder of the molecule.

In some embodiments, V is CH; Y is N; G is CH₂ and G is connected with the nitrogen of Ring A; Ring A is

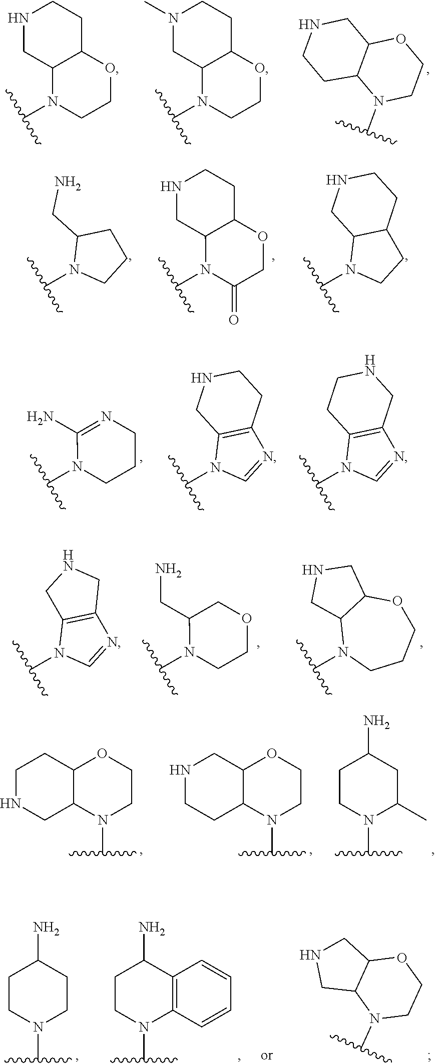

R¹ is ethyl, isopropyl, —CH₂—CHF₂, or

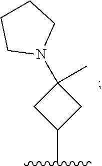 [SMILES: *C1CC(C)(N2CCCC2)C1]

and the spiro moiety represented by the below formula:

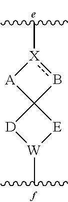

is

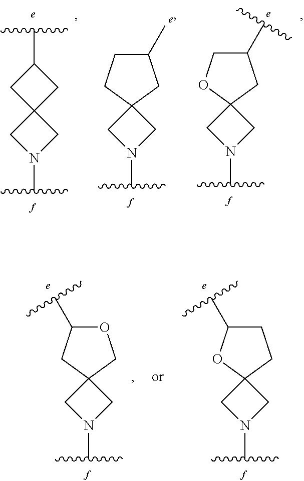

wherein e and f indicate points of attachment to the remainder of the molecule.

In some embodiments, V is CH; Y is N; G is CH₂ and G is connected with the nitrogen of Ring A; Ring A is

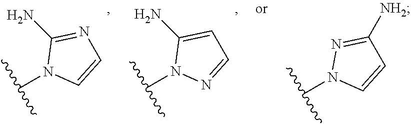 [SMILES: *n1ccnc1N ; *n1nccc1N ; *n1ccc(N)n1]

R¹ is ethyl, isopropyl, —CH₂—CHF₂,

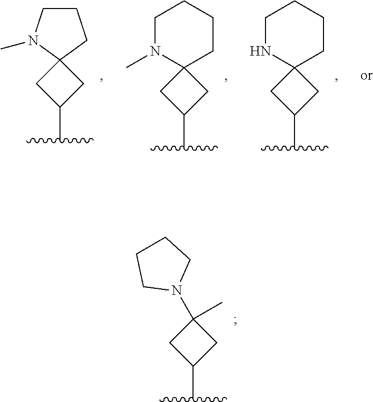

and the spiro moiety represented by the below formula:

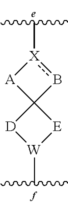

is

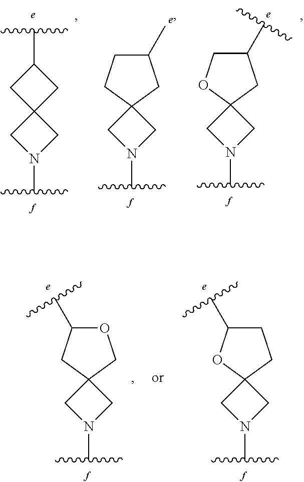

wherein e and f indicate points of attachment to the remainder of the molecule.

In some embodiments, V is CH; Y is N; G is CH₂ and G is connected with the nitrogen of Ring A; Ring A is

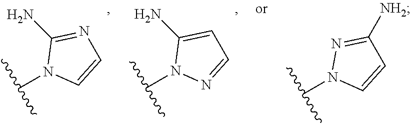 [SMILES: *n1ccnc1N ; *n1nccc1N ; *n1ccc(N)n1]

R¹ is ethyl, isopropyl, —CH₂—CHF₂, or

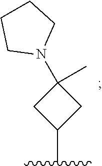 [SMILES: *C1CC(C)(N2CCCC2)C1]

and the spiro moiety represented by the below formula:

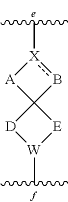

is

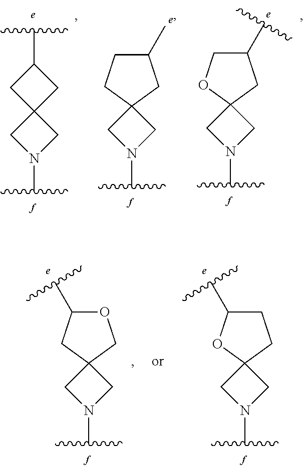

wherein e and f indicate points of attachment to the remainder of the molecule.

In some embodiments, V is CH, Y is N, G is CH₂ and G is connected with the nitrogen of Ring A; R¹ is ethyl, isopropyl, —CH₂—CHF₂,

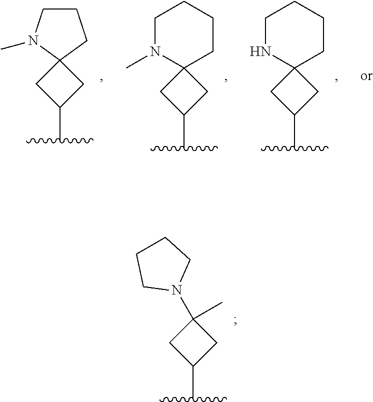

R² is isopropyl; and the spiro moiety represented by the below formula:

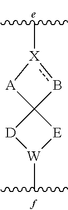

is

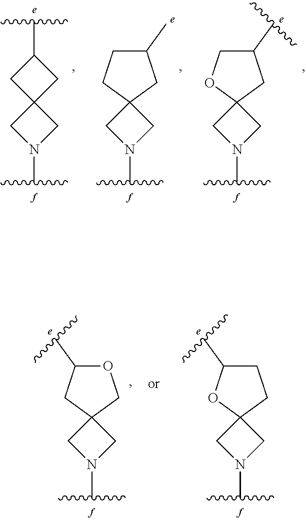

wherein e and f indicate points of attachment to the remainder of the molecule.

In some embodiments, V is CH, Y is N, G is CH₂ and G is connected with the nitrogen of Ring A; R¹ is ethyl, isopropyl, —CH₂—CHF₂, or

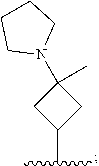 [SMILES: *C1CC(C)(N2CCCC2)C1]

R² is isopropyl; and the spiro moiety represented by the below formula:

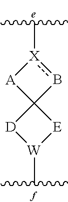

is

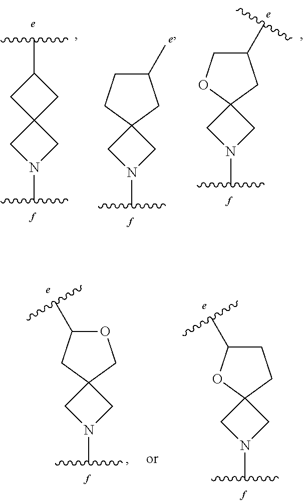

wherein e and f indicate points of attachment to the remainder of the molecule.

In some embodiments, V is CH; Y is N; G is CH₂ and G is connected with the nitrogen of Ring A; Ring A is

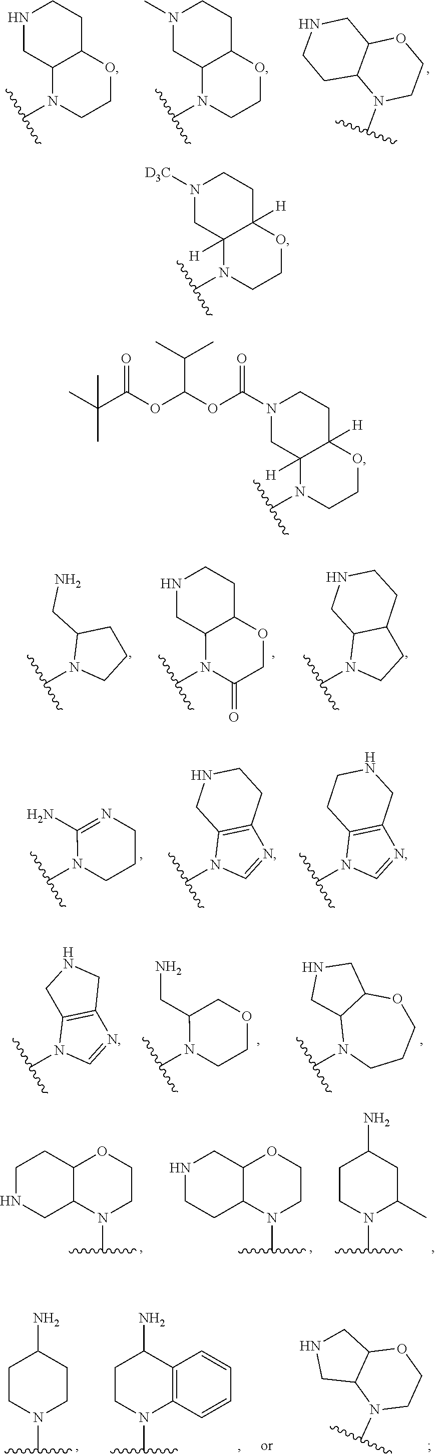

R¹ is ethyl, isopropyl, —CH₂—CHF₂,

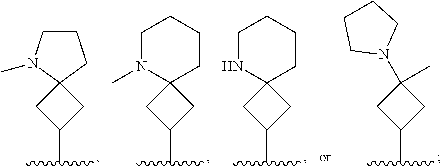

R² is isopropyl; and the spiro moiety represented by the below formula:

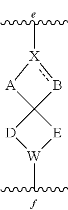

is

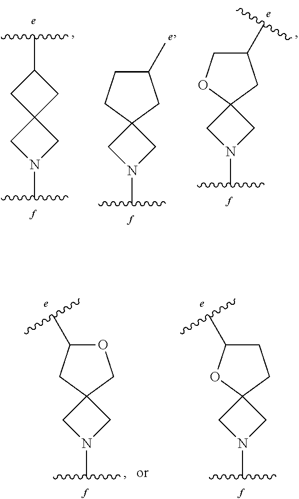

wherein e and f indicate points of attachment to the remainder of the molecule.

In some embodiments, V is CH; Y is N; G is CH₂ and G is connected with the nitrogen of Ring A; Ring A is

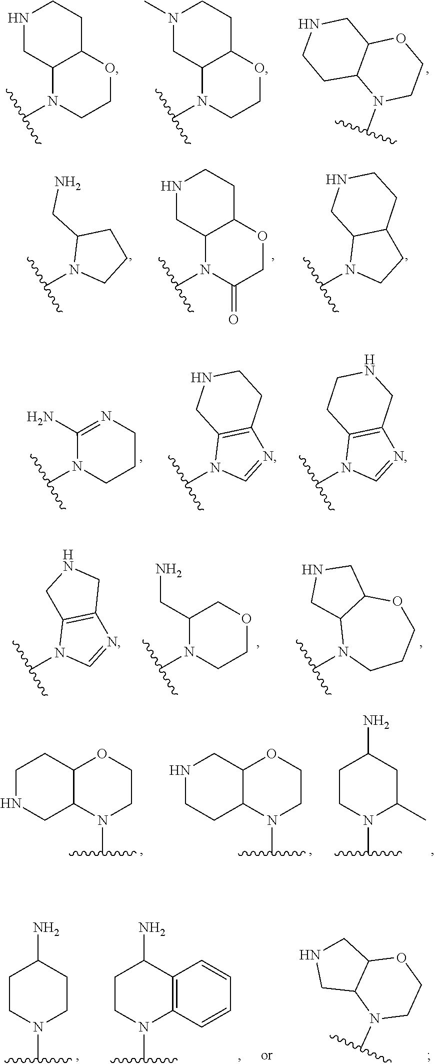

R¹ is ethyl, isopropyl, —CH₂—CHF₂,

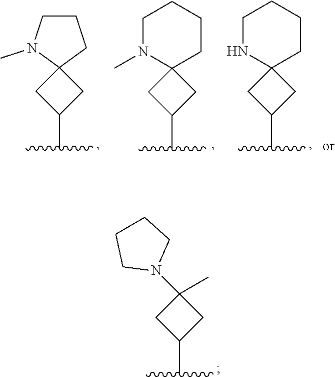

R² is isopropyl; and the spiro moiety represented by the below formula:

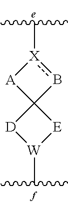

is

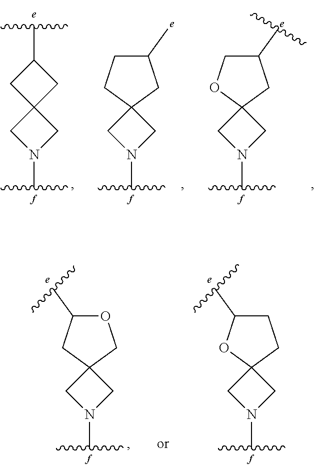

wherein e and f indicate points of attachment to the remainder of the molecule.

In some embodiments, V is CH; Y is N; G is CH₂ and G is connected with the nitrogen of Ring A; Ring A is

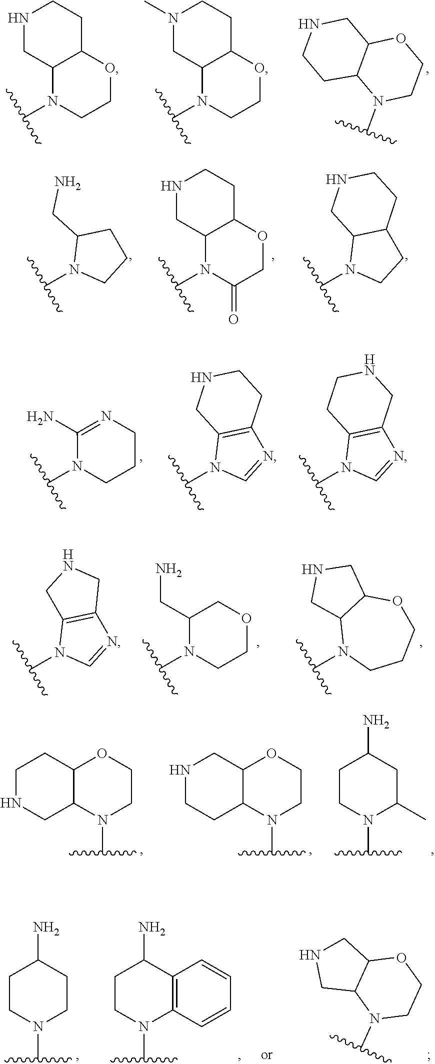

R¹ is ethyl, isopropyl, —CH₂—CHF₂, or

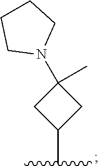 [SMILES: *C1CC(C)(N2CCCC2)C1]

R² is isopropyl; and the spiro moiety represented by the below formula:

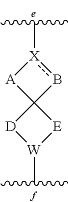

is

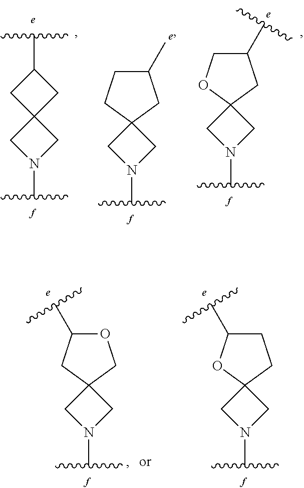

wherein e and f indicate points of attachment to the remainder of the molecule.

In some embodiments, V is CH; Y is N; G is CH₂ and G is connected with the nitrogen of Ring A; Ring A is

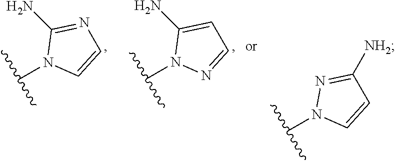 [SMILES: *n1ccnc1N ; *n1nccc1N ; *n1ccc(N)n1]

R¹ is ethyl, isopropyl, —CH₂—CHF₂,

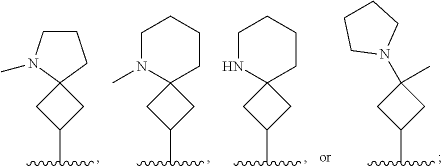

R² is isopropyl; and the spiro moiety represented by the below formula:

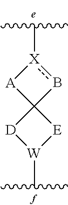

is

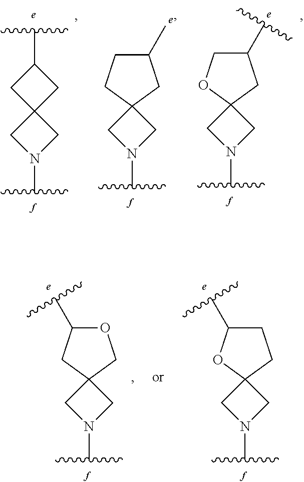

wherein e and f indicate points of attachment to the remainder of the molecule.

In some embodiments, V is CH; Y is N; G is CH₂ and G is connected with the nitrogen of Ring A; Ring A is

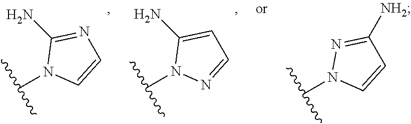 [SMILES: *n1ccnc1N ; *n1nccc1N ; *n1ccc(N)n1]

R¹ is ethyl, isopropyl, —CH₂—CHF₂, or

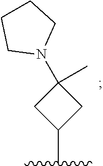 [SMILES: *C1CC(C)(N2CCCC2)C1]

R² is isopropyl; and the spiro moiety represented by the below formula:

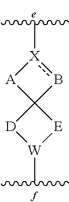

is

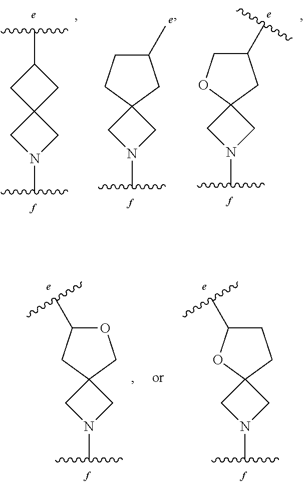

wherein e and f indicate points of attachment to the remainder of the molecule.

In some embodiments, V is CH; Y is N; G is CH₂ and G is connected with the nitrogen of Ring A; R¹ and R² form

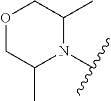 [SMILES: *N1C(C)COCC1C]

group with the nitrogen to which they are connected; and the spiro moiety represented by the below formula:

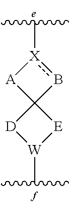

is

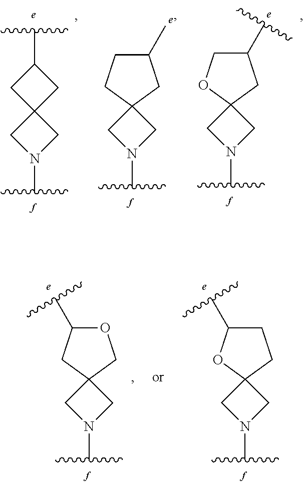

wherein e and f indicate points of attachment to the remainder of the molecule.

In some embodiments, V is CH; Y is N; G is CH₂ and G is connected with the nitrogen of Ring A; Ring A is

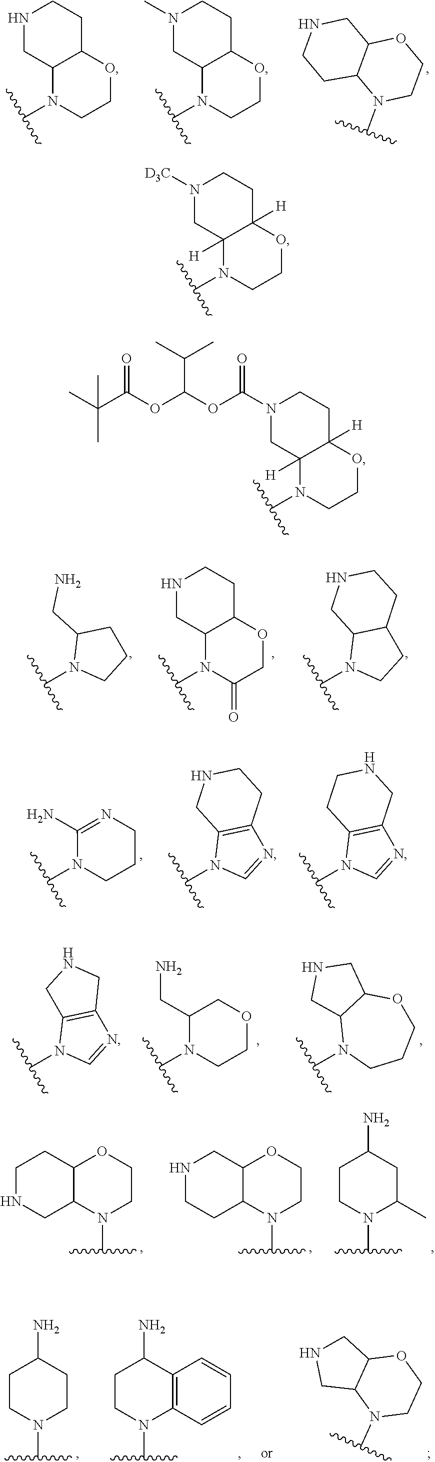

R¹ and R² form

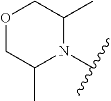 [SMILES: *N1C(C)COCC1C]

group with the nitrogen to which they are connected; and the spiro moiety represented by the below formula:

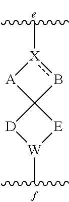

is

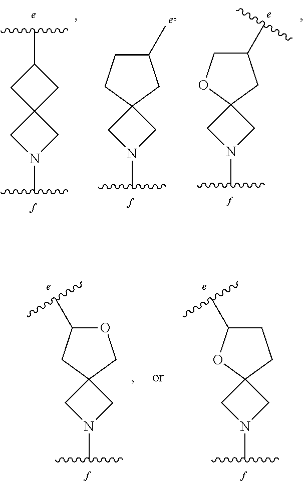

wherein e and f indicate points of attachment to the remainder of the molecule.

In some embodiments, V is CH; Y is N; G is CH₂ and G is connected with the nitrogen of Ring A; Ring A is

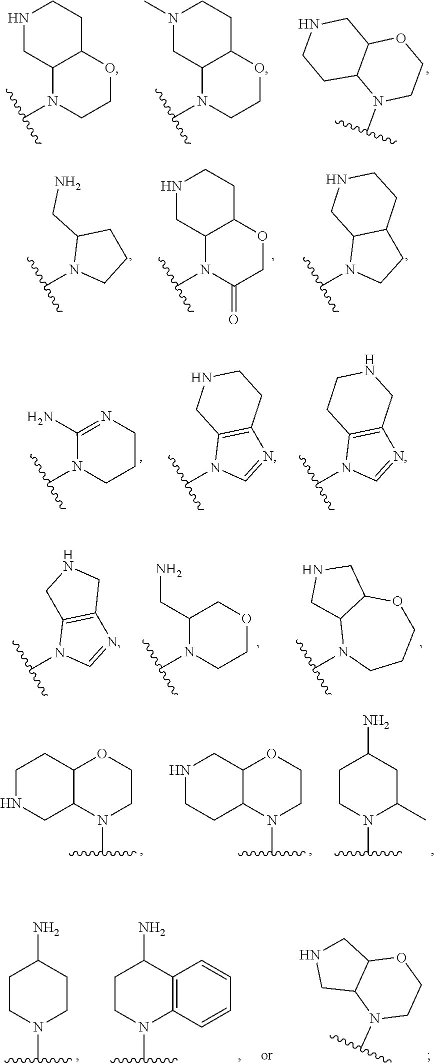

R¹ and R² form

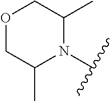 [SMILES: *N1C(C)COCC1C]

group with the nitrogen to which they are connected; and the spiro moiety represented by the below formula:

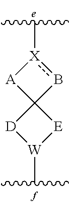

is

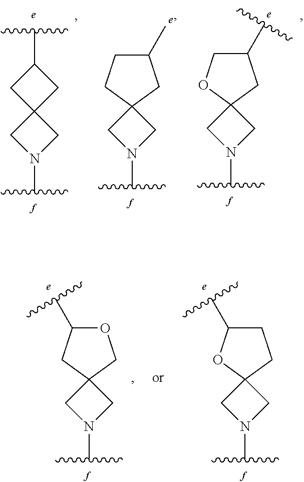

wherein e and f indicate points of attachment to the remainder of the molecule.

In some embodiments, V is CH; Y is N; G is CH₂ and G is connected with the nitrogen of Ring A; Ring A is

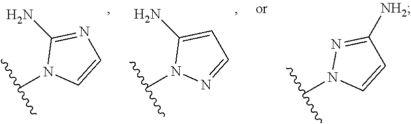 [SMILES: *n1ccnc1N ; *n1nccc1N ; *n1ccc(N)n1]

R¹ and R² form

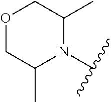 [SMILES: *N1C(C)COCC1C]

group with the nitrogen to which they are connected; and the spiro moiety represented by the below formula:

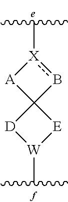

is

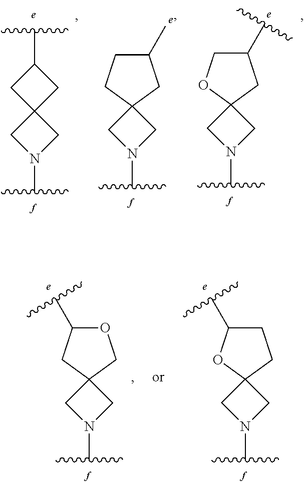

wherein e and f indicate points of attachment to the remainder of the molecule.

In some embodiments, V is CH, Y is N, and G is absent and X is connected with a nitrogen of Ring A.

In some embodiments, V is CH; Y is N; G is absent and X is connected with a nitrogen of Ring A; and Ring A is

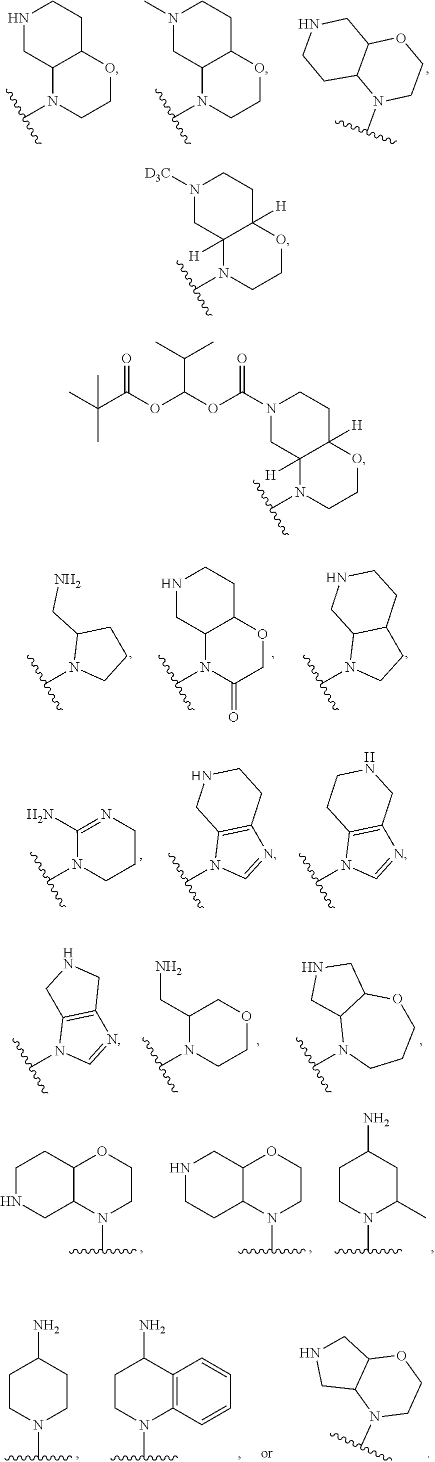

In some embodiments, V is CH; Y is N; G is absent and X is connected with a nitrogen of Ring A; and Ring A is

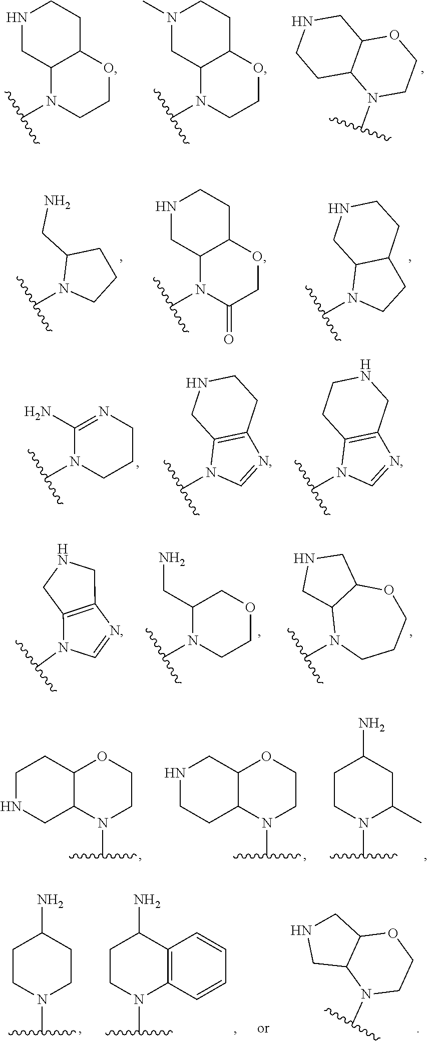

In some embodiments, V is CH; Y is N; G is absent and X is connected with a nitrogen of Ring A; and Ring A is

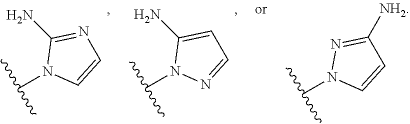 [SMILES: *n1ccnc1N ; *n1nccc1N ; *n1ccc(N)n1]

In some embodiments, V is CH, Y is N, G is absent and X is connected with a nitrogen of Ring A, and R² is isopropyl.

In some embodiments, V is CH; Y is N; G is absent and X is connected with a nitrogen of Ring A; Ring A is

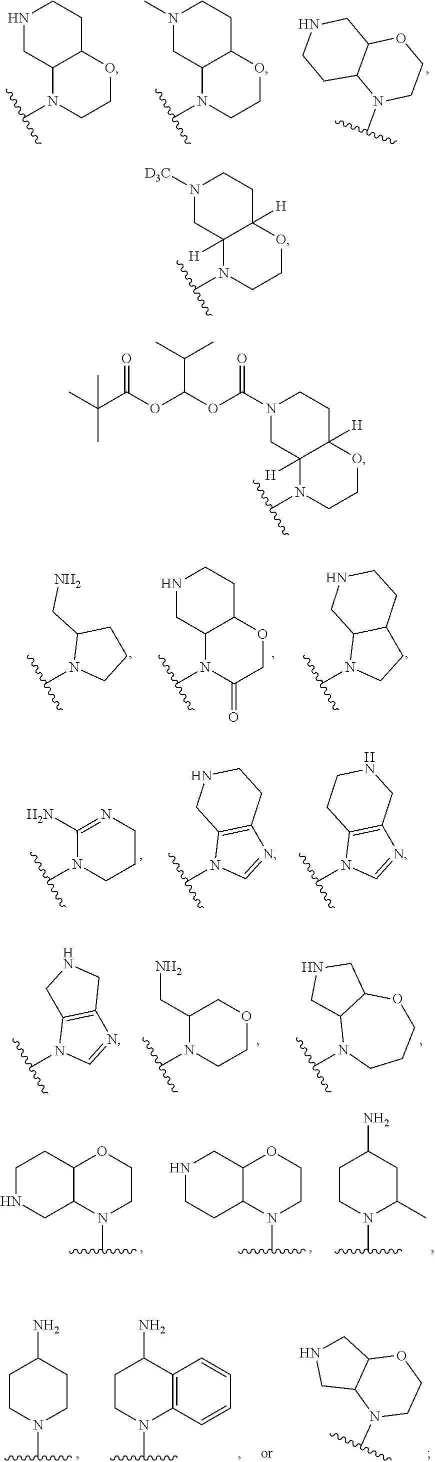

and R² is isopropyl.

In some embodiments, V is CH; Y is N; G is absent and X is connected with a nitrogen of Ring A; Ring A is

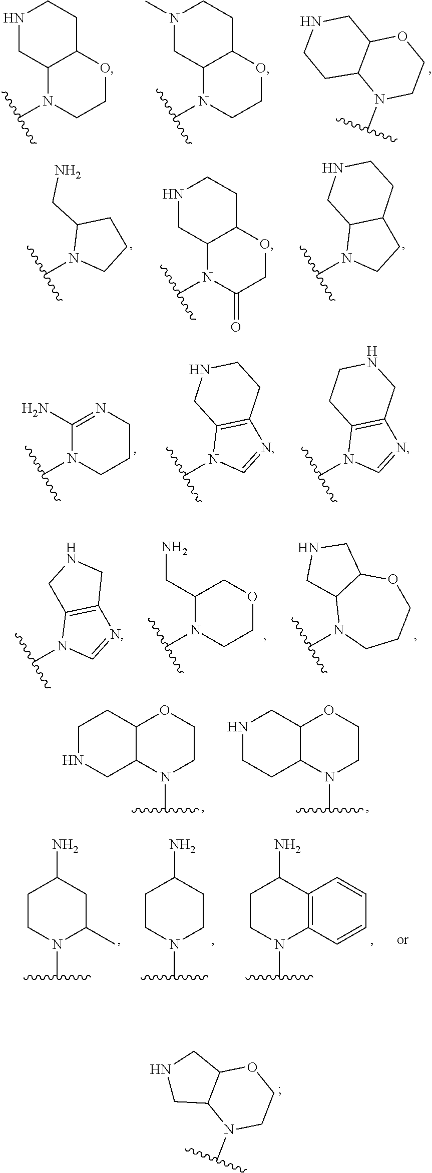

and R² is isopropyl.

In some embodiments, V is CH; Y is N; G is absent and X is connected with a nitrogen of Ring A; Ring A is

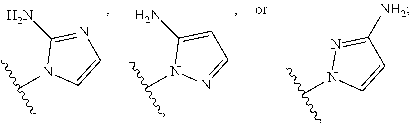 [SMILES: *n1ccnc1N ; *n1nccc1N ; *n1ccc(N)n1]

and R² is isopropyl.

In some embodiments, V is CH; Y is N; G is absent and X is connected with a nitrogen of Ring A; and R¹ is ethyl, isopropyl, —CH₂—CHF₂,

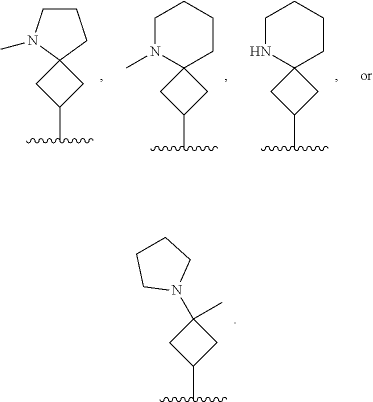

In some embodiments, V is CH; Y is N; G is absent and X is connected with a nitrogen of Ring A; and R¹ is ethyl, isopropyl, —CH₂—CHF₂, or

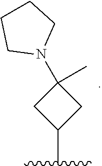 [SMILES: *C1CC(C)(N2CCCC2)C1]

In some embodiments, V is CH; Y is N; G is absent and X is connected with a nitrogen of Ring A; Ring A is

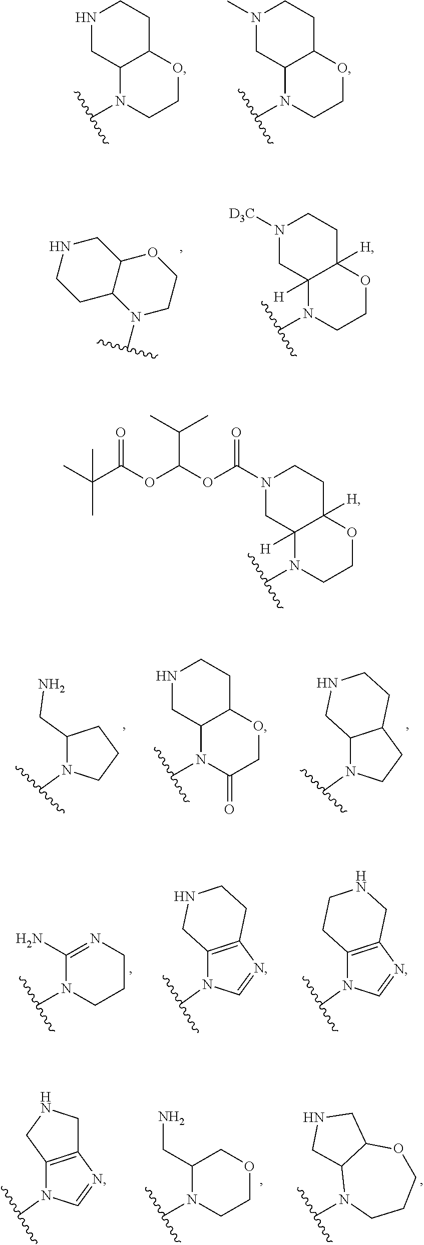

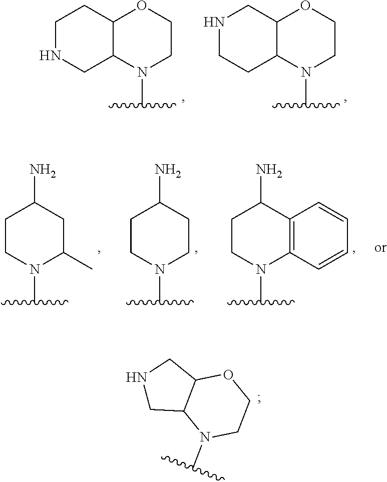

and R¹ is ethyl, isopropyl, —CH₂—CHF₂,

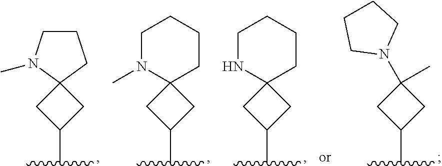

In some embodiments, V is CH; Y is N; G is absent and X is connected with a nitrogen of Ring A; Ring A is

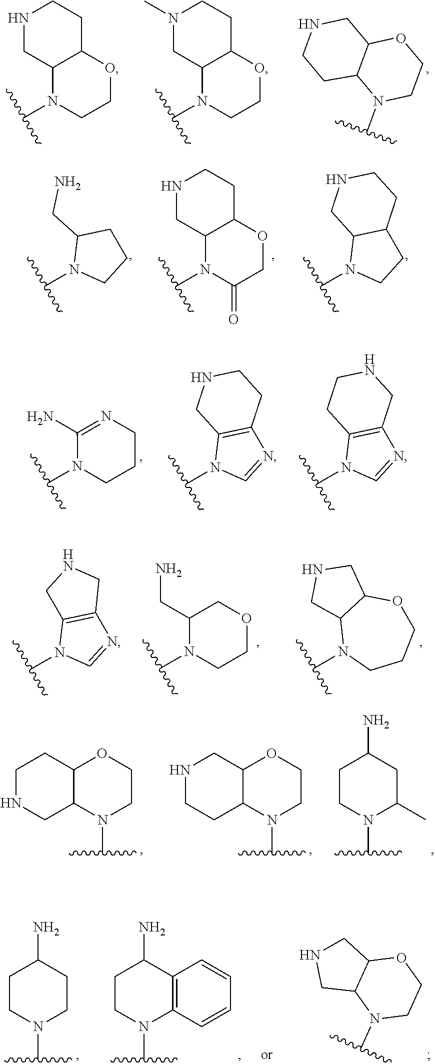

and R¹ is ethyl, isopropyl, —CH₂—CHF₂,

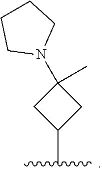 [SMILES: *C1CC(C)(N2CCCC2)C1]

In some embodiments, V is CH; Y is N; G is absent and X is connected with a nitrogen of Ring A; Ring A is

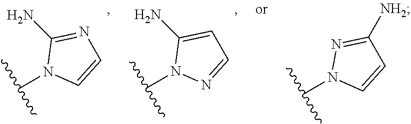 [SMILES: *n1ccnc1N ; *n1nccc1N ; *n1ccc(N)n1]

and R¹ is ethyl, isopropyl, —CH₂—CHF₂,

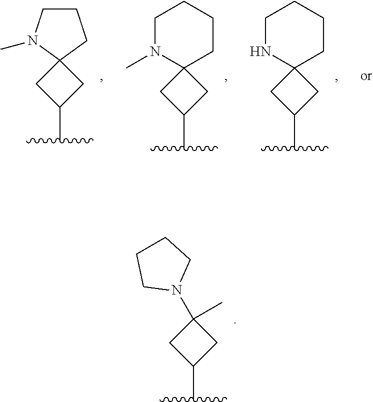

In some embodiments, V is CH; Y is N; G is absent and X is connected with a nitrogen of Ring A; Ring A is

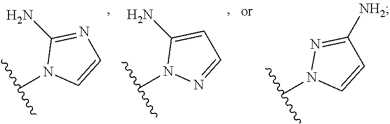 [SMILES: *n1ccnc1N ; *n1nccc1N ; *n1ccc(N)n1]

and R¹ is ethyl, isopropyl, —CH₂—CHF₂, or

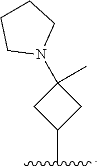 [SMILES: *C1CC(C)(N2CCCC2)C1]

In some embodiments, V is CH, Y is N, G is absent and X is connected with a nitrogen of Ring A; R¹ is ethyl, isopropyl, —CH₂—CHF₂,

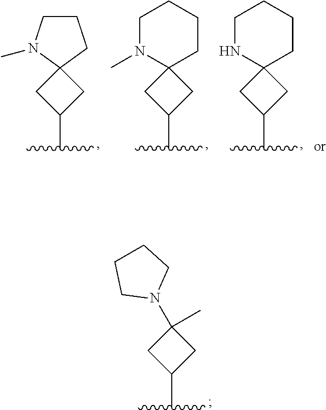

and R² is isopropyl.

In some embodiments, V is CH, Y is N, G is absent and X is connected with a nitrogen of Ring A; R¹ is ethyl, isopropyl, —CH₂—CHF₂, or

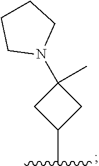 [SMILES: *C1CC(C)(N2CCCC2)C1]

and R² is isopropyl.

In some embodiments, V is CH; Y is N; G is absent and X is connected with a nitrogen of Ring A; Ring A is

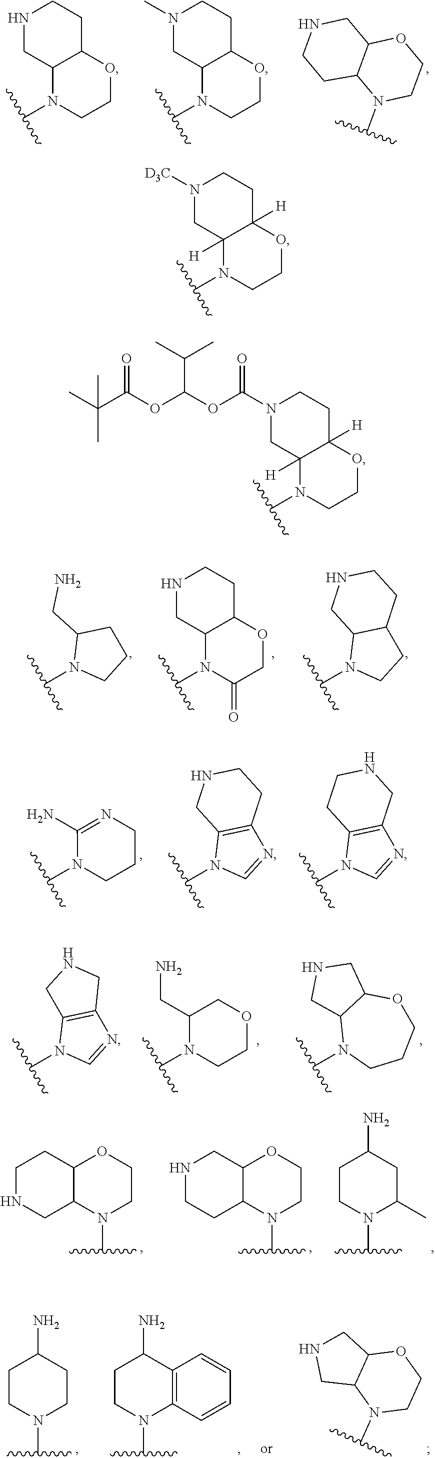

R¹ is ethyl, isopropyl, —CH₂—CHF₂,

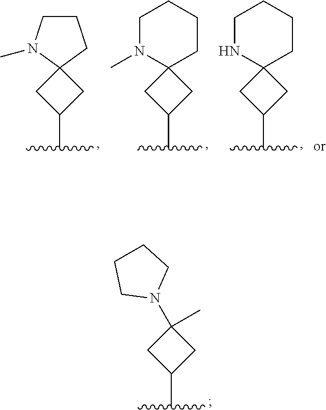

and R² is isopropyl.

In some embodiments, V is CH; Y is N; G is absent and X is connected with a nitrogen of Ring A; Ring A is

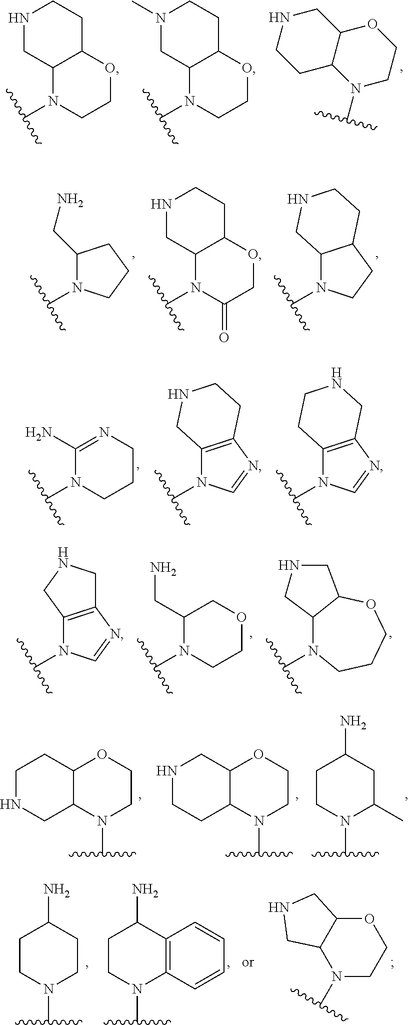

R¹ is ethyl, isopropyl, —CH₂—CHF₂, or

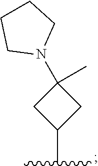 [SMILES: *C1CC(C)(N2CCCC2)C1]

and R² is isopropyl.

In some embodiments, V is CH; Y is N; G is absent and X is connected with a nitrogen of Ring A; Ring A is

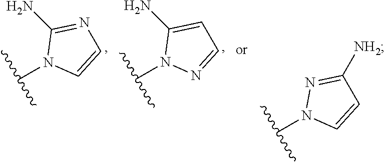

R¹ is ethyl, isopropyl, —CH₂—CHF₂,

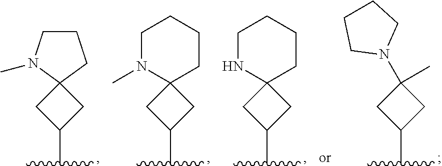

and R² is isopropyl.

In some embodiments, V is CH; Y is N; G is absent and X is connected with a nitrogen of Ring A; Ring A is

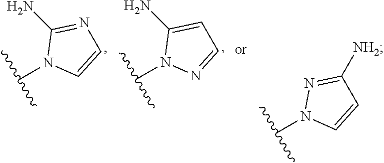

R¹ is ethyl, isopropyl, —CH₂—CHF₂, or

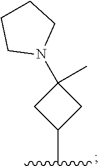 [SMILES: *C1CC(C)(N2CCCC2)C1]

and R² is isopropyl.

In some embodiments, V is CH, Y is N, G is absent and X is connected with a nitrogen of Ring A, and R¹ and R² form

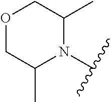 [SMILES: *N1C(C)COCC1C]

group with the nitrogen to which they are connected.

In some embodiments, V is CH; Y is N; G is absent and X is connected with a nitrogen of Ring A; Ring A is

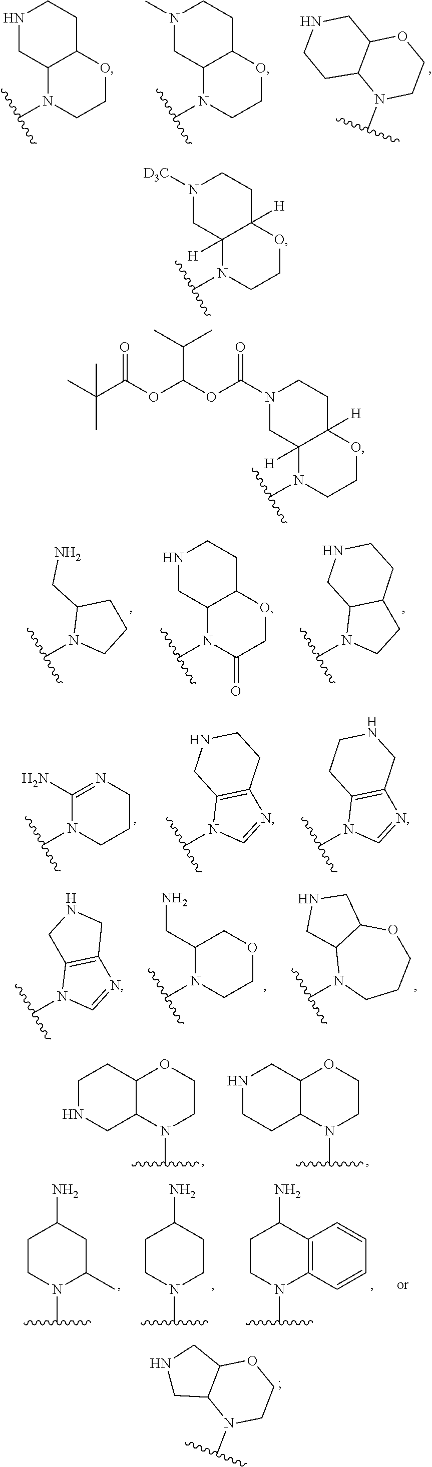

and R¹ and R² form

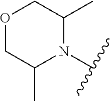 [SMILES: *N1C(C)COCC1C]

group with the nitrogen to which they are connected.

In some embodiments, V is CH; Y is N; G is absent and X is connected with a nitrogen of Ring A; Ring A is

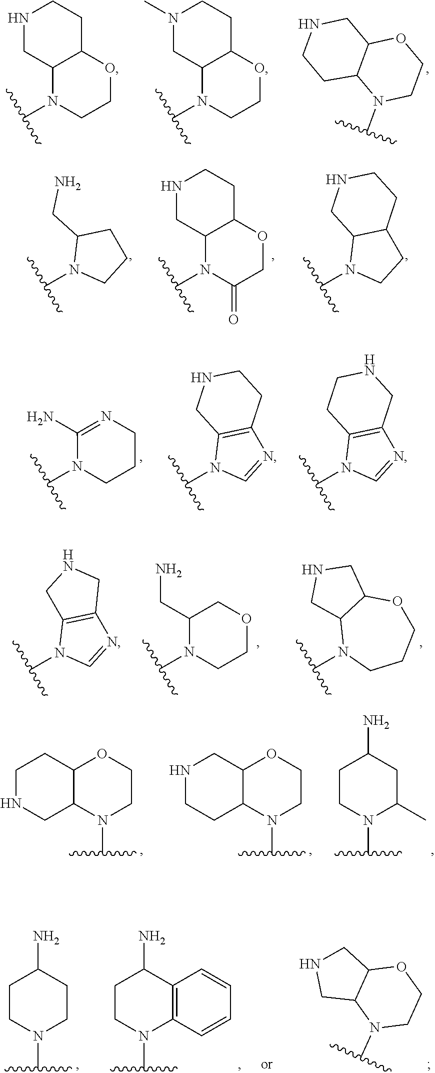

and R¹ and R² form

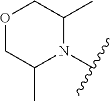 [SMILES: *N1C(C)COCC1C]

group with the nitrogen to which they are connected.

In some embodiments, V is CH; Y is N; G is absent and X is connected with a nitrogen of Ring A; Ring A is

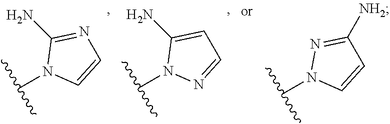 [SMILES: *n1ccnc1N ; *n1nccc1N ; *n1ccc(N)n1]

and R¹ and R² form

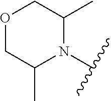 [SMILES: *N1C(C)COCC1C]

group with the nitrogen to which they are connected.

In some embodiments, V is CH, Y is N, G is absent and X is connected with a nitrogen of Ring A; and the spiro moiety represented by the below formula:

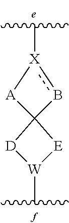

is

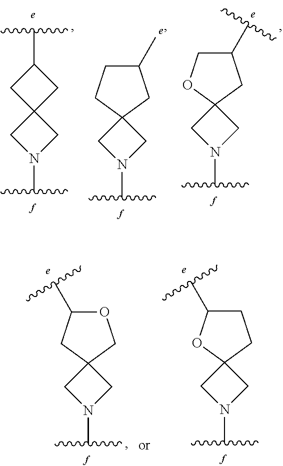

wherein e and f indicate points of attachment to the remainder of the molecule.

In some embodiments, V is CH; Y is N; G is absent and X is connected with a nitrogen of Ring A; Ring A is

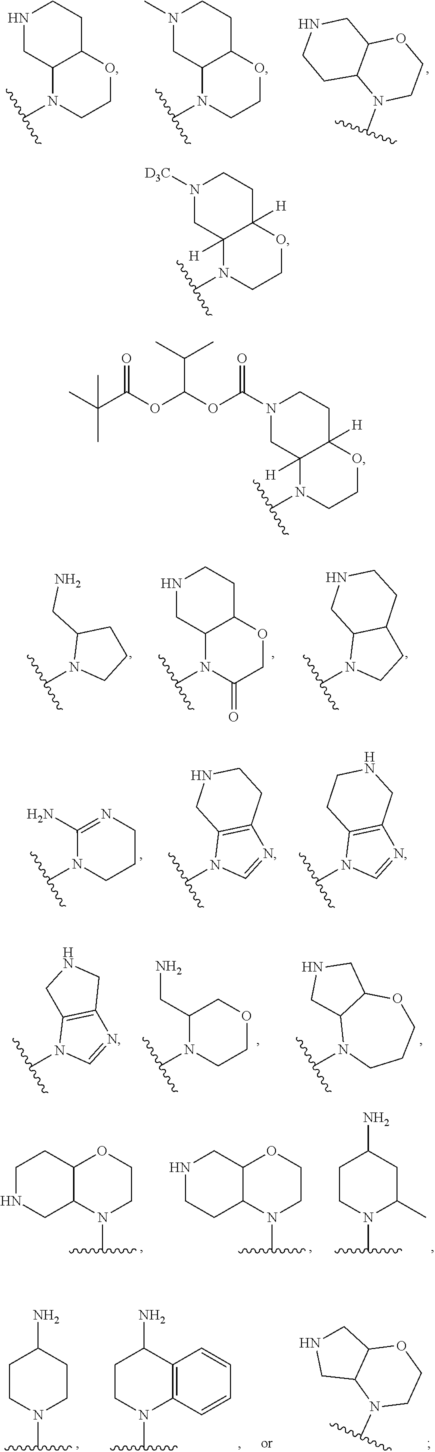

and the spiro moiety represented by the below formula:

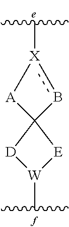

is

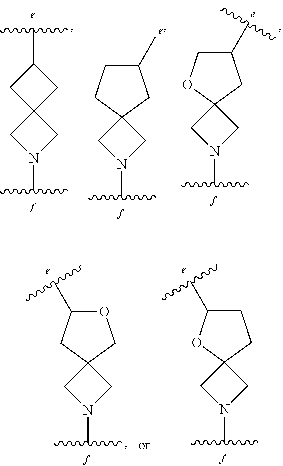

wherein e and f indicate points of attachment to the remainder of the molecule.

In some embodiments, V is CH; Y is N; G is absent and X is connected with a nitrogen of Ring A; Ring A is

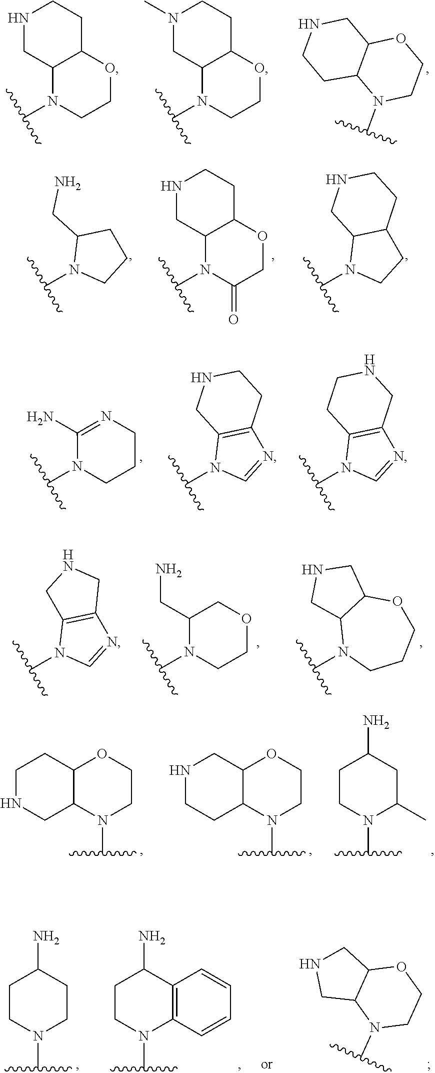

and the spiro moiety represented by the below formula:

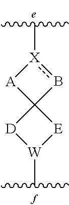

is

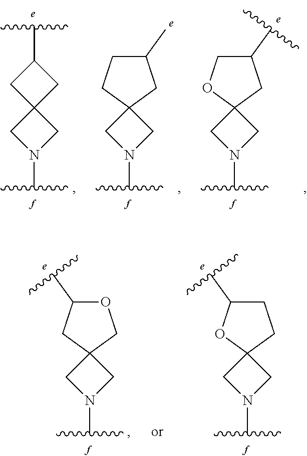

wherein e and f indicate points of attachment to the remainder of the molecule.

In some embodiments, V is CH; Y is N; G is absent and X is connected with a nitrogen of Ring A; Ring A is

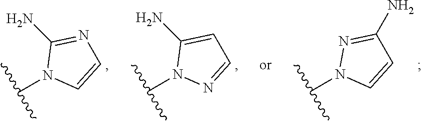 [SMILES: *n1ccnc1N ; *n1nccc1N ; *n1ccc(N)n1]

and the spiro moiety represented by the below formula:

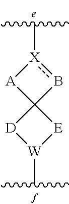

is

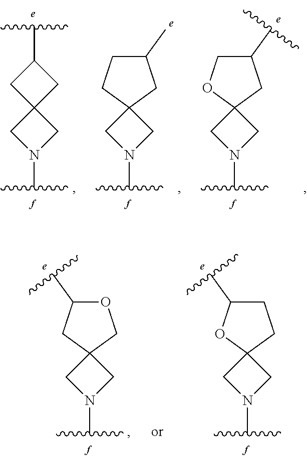

wherein e and f indicate points of attachment to the remainder of the molecule.

In some embodiments, V is CH, Y is N, G is absent and X is connected with a nitrogen of Ring A, R² is isopropyl; and the spiro moiety represented by the below formula:

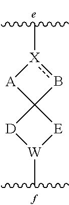

is

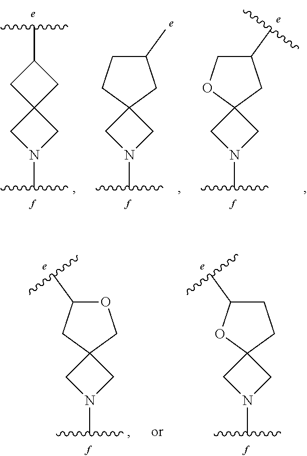

wherein e and f indicate points of attachment to the remainder of the molecule.

In some embodiments, V is CH; Y is N; G is absent and X is connected with a nitrogen of Ring A; Ring A is

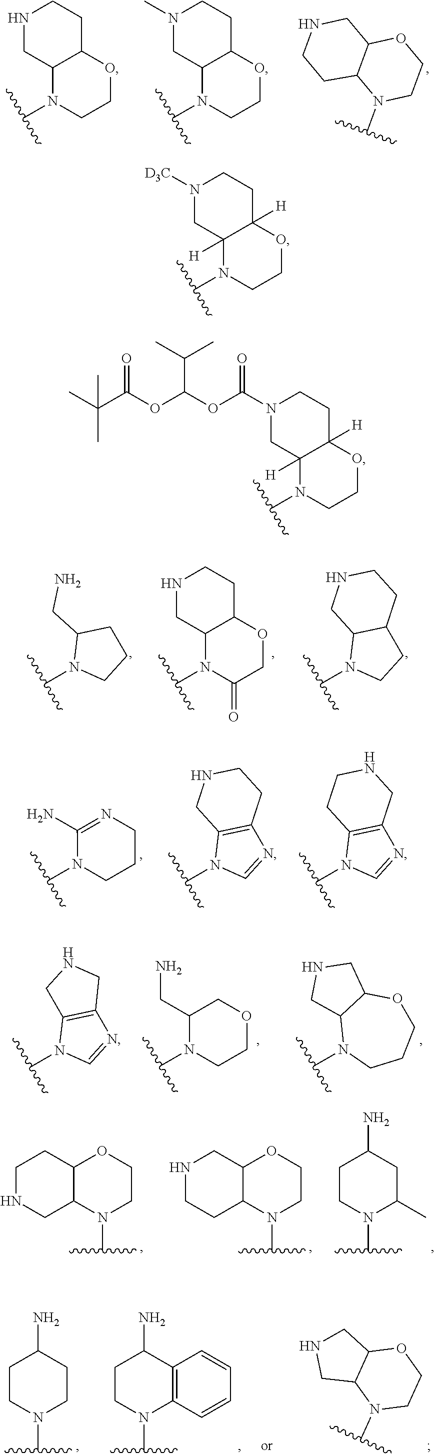

R² is isopropyl; and the spiro moiety represented by the below formula:

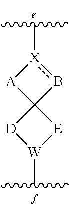

is

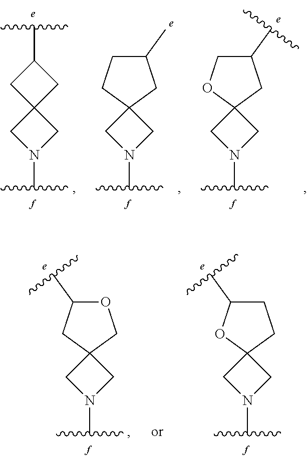

wherein e and f indicate points of attachment to the remainder of the molecule.

In some embodiments, V is CH; Y is N; G is absent and X is connected with a nitrogen of Ring A; Ring A is

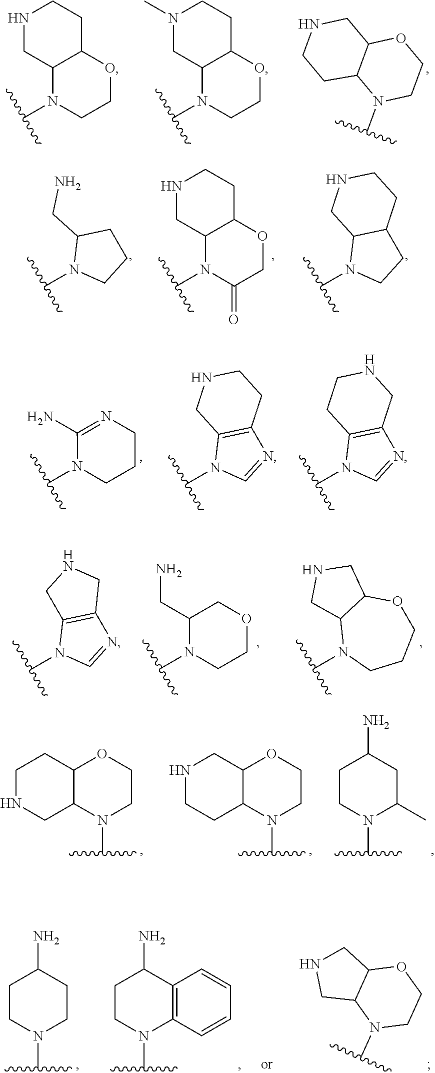

R² is isopropyl; and the spiro moiety represented by the below formula:

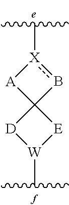

is

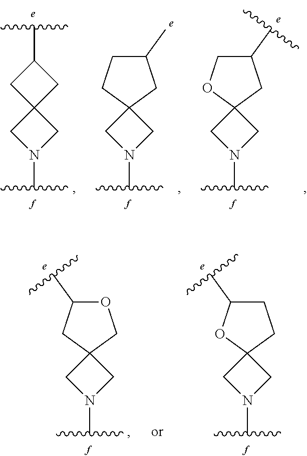

wherein e and f indicate points of attachment to the remainder of the molecule.

In some embodiments, V is CH; Y is N; G is absent and X is connected with a nitrogen of Ring A; Ring A is

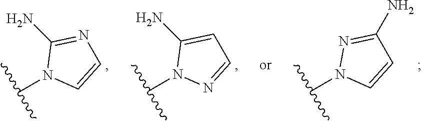 [SMILES: *n1ccnc1N ; *n1nccc1N ; *n1ccc(N)n1]

R² is isopropyl; and the spiro moiety represented by the below formula:

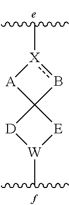

is

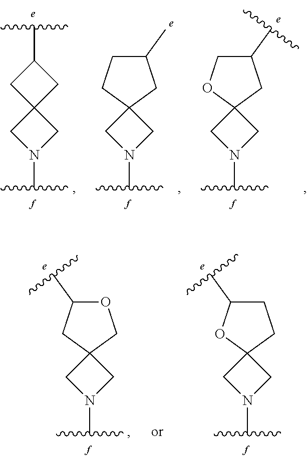

wherein e and f indicate points of attachment to the remainder of the molecule.

In some embodiments, V is CH; Y is N; G is absent and X is connected with a nitrogen of Ring A; R¹ is ethyl, isopropyl, —CH₂—CHF₂,

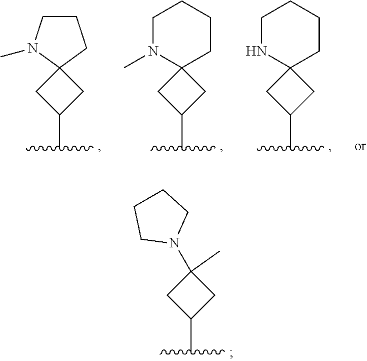

and the spiro moiety represented by the below formula:

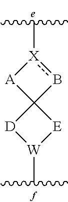

is

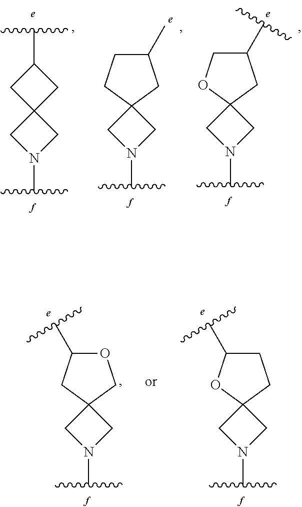

wherein e and f indicate points of attachment to the remainder of the molecule.

In some embodiments, V is CH; Y is N; G is absent and X is connected with a nitrogen of Ring A; R¹ is ethyl, isopropyl, —CH₂—CHF₂, or

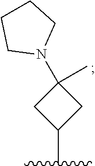 [SMILES: *C1CC(C)(N2CCCC2)C1]

and the spiro moiety represented by the below formula:

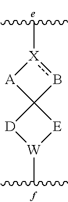

is

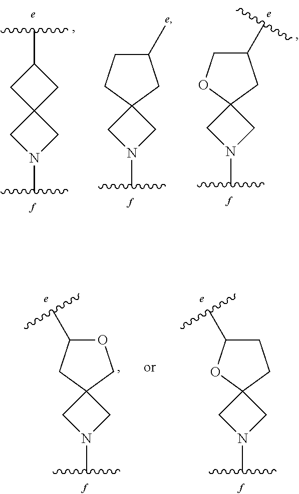

wherein e and f indicate points of attachment to the remainder of the molecule.

In some embodiments, V is CH; Y is N; G is absent and X is connected with a nitrogen of Ring A; Ring A is

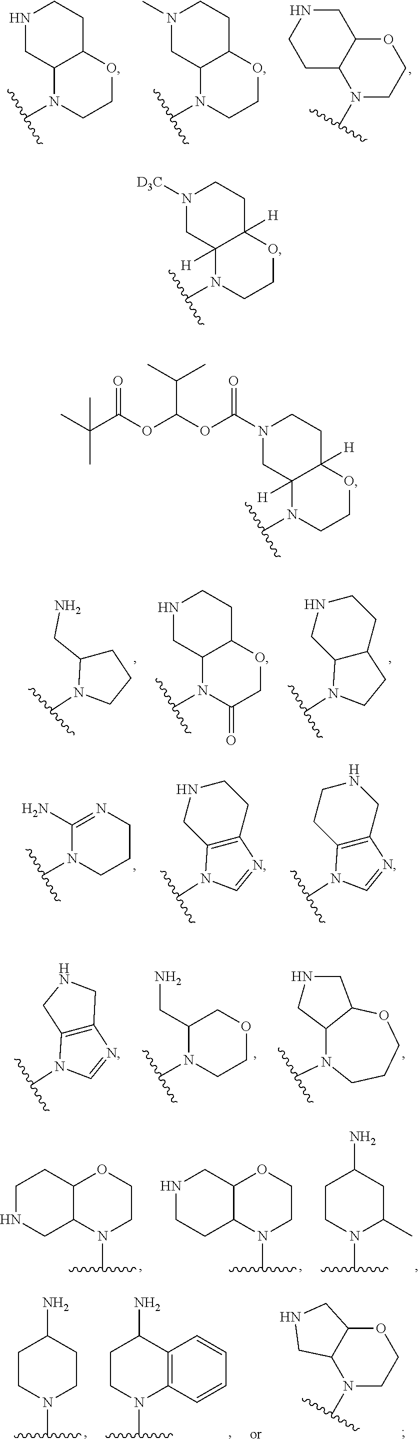

R¹ is ethyl, isopropyl, —CH₂—CHF₂,

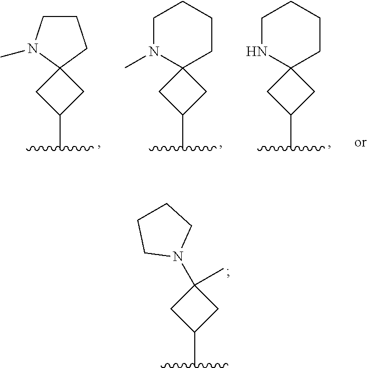

and the spiro moiety represented by the below formula:

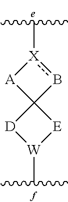

is

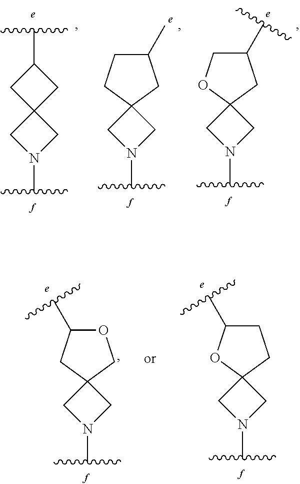

wherein e and f indicate points of attachment to the remainder of the molecule.

In some embodiments, V is CH; Y is N; G is absent and X is connected with a nitrogen of Ring A; Ring A is

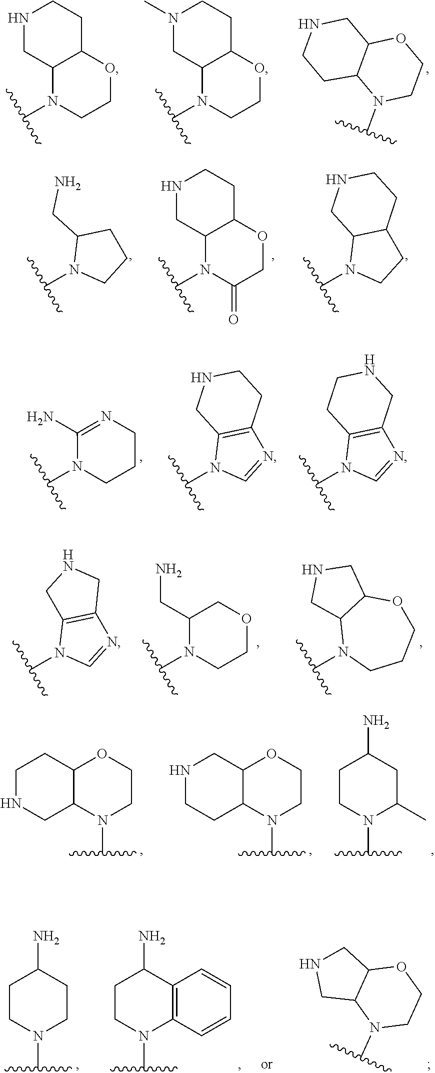

R¹ is ethyl, isopropyl, —CH₂—CHF₂, or

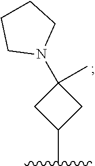 [SMILES: *C1CC(C)(N2CCCC2)C1]

and the spiro moiety represented by the below formula:

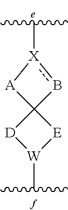

is

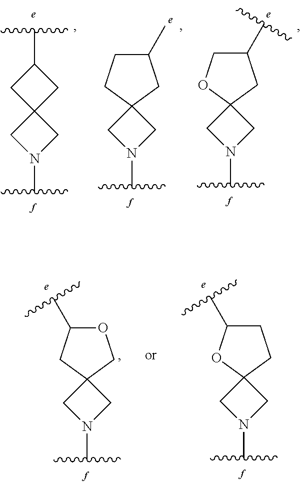

wherein e and f indicate points of attachment to the remainder of the molecule.

In some embodiments, V is CH; Y is N; G is absent and X is connected with a nitrogen of Ring A; Ring A is

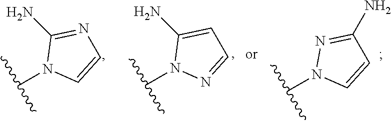 [SMILES: *n1ccnc1N ; *n1nccc1N ; *n1ccc(N)n1]

R¹ is ethyl, isopropyl, —CH₂—CHF₂,

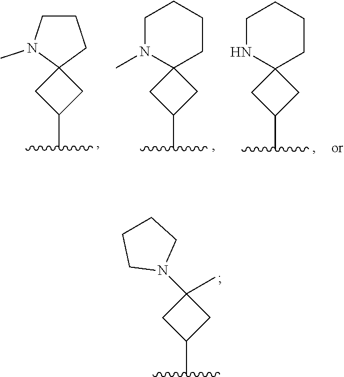

and the spiro moiety represented by the below formula:

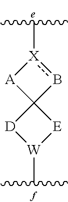

is

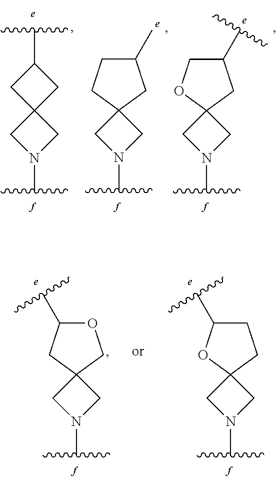

wherein e and f indicate points of attachment to the remainder of the molecule.

In some embodiments, V is CH; Y is N; G is absent and X is connected with a nitrogen of Ring A; Ring A is

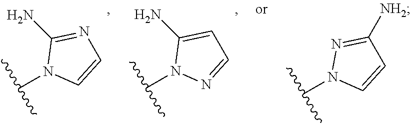 [SMILES: *n1ccnc1N ; *n1nccc1N ; *n1ccc(N)n1]

R¹ is ethyl, isopropyl, —CH₂—CHF₂, or

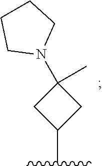 [SMILES: *C1CC(C)(N2CCCC2)C1]

and the spiro moiety represented by the below formula:

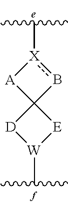

is

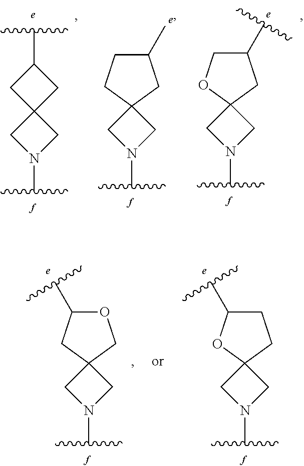

wherein e and f indicate points of attachment to the remainder of the molecule.

In some embodiments, V is CH, Y is N, G is absent and X is connected with a nitrogen of Ring A; R¹ is ethyl, isopropyl, —CH₂—CHF₂,

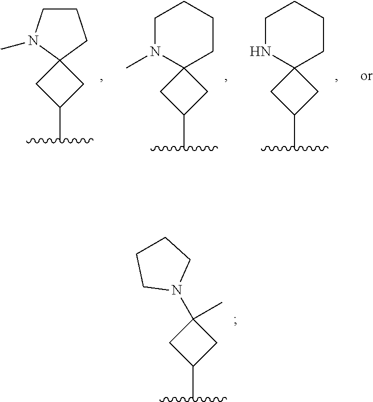

R² is isopropyl; and the spiro moiety represented by the below formula:

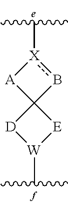

is

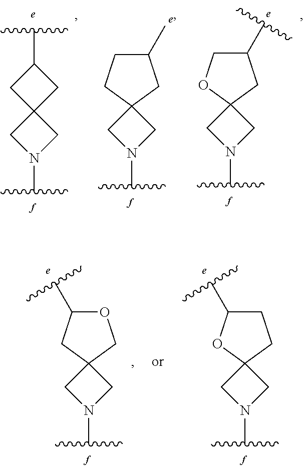

or wherein e and f indicate points of attachment to the remainder of the molecule.

In some embodiments, V is CH, Y is N, G is absent and X is connected with a nitrogen of Ring A; R¹ is ethyl, isopropyl, —CH₂—CHF₂, or

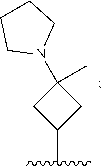 [SMILES: *C1CC(C)(N2CCCC2)C1]

R² is isopropyl; and the spiro moiety represented by the below formula:

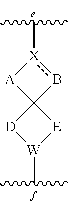

is

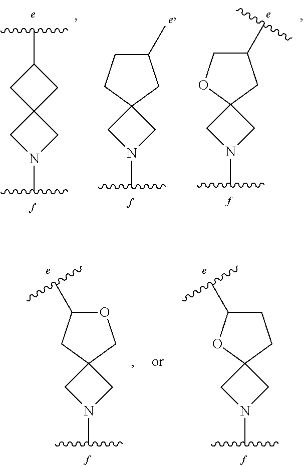

wherein e and f indicate points of attachment to the remainder of the molecule.

In some embodiments, V is CH; Y is N; G is absent and X is connected with a nitrogen of Ring A; Ring A is

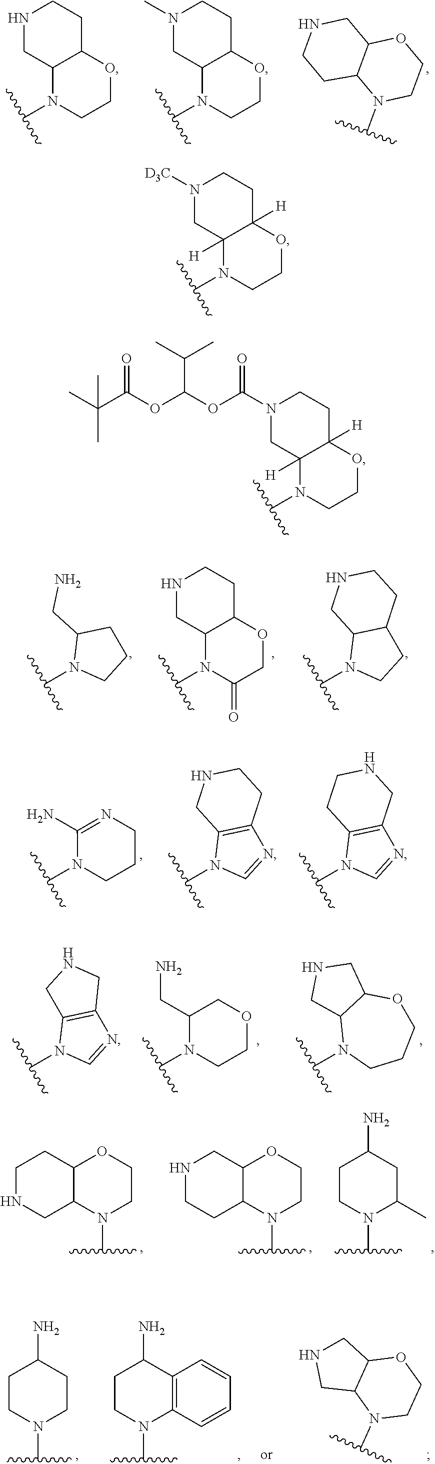

R¹ is ethyl, isopropyl, —CH₂—CHF₂,

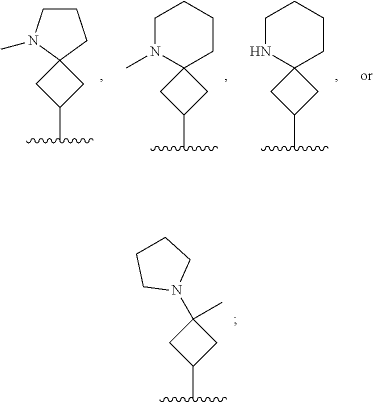

R² is isopropyl; and the spiro moiety represented by the below formula:

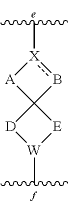

is

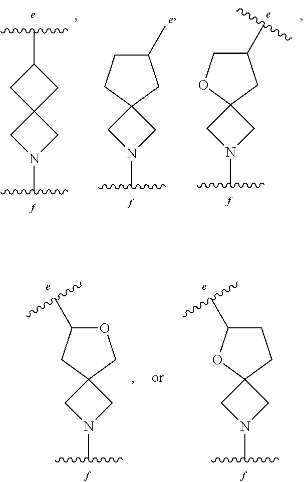

wherein e and f indicate points of attachment to the remainder of the molecule.

In some embodiments, V is CH; Y is N; G is absent and X is connected with a nitrogen of Ring A; Ring A is

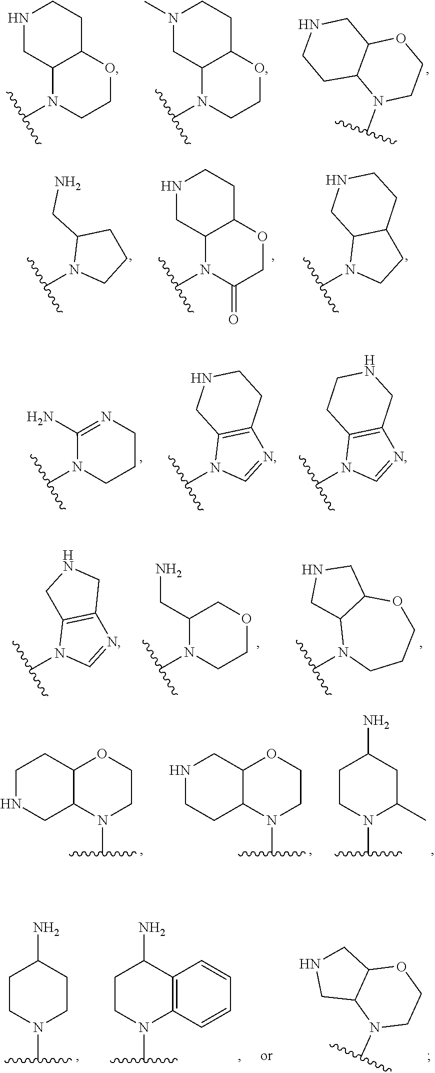

R¹ is ethyl, isopropyl, —CH₂—CHF₂, or

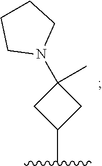 [SMILES: *C1CC(C)(N2CCCC2)C1]

R² is isopropyl; and the spiro moiety represented by the below formula:

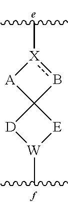

is

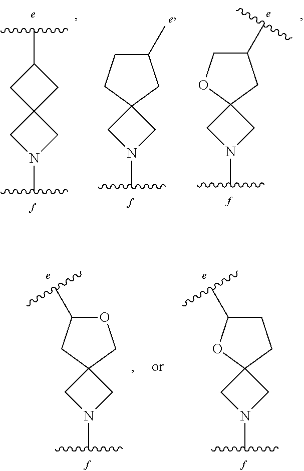

wherein e and f indicate points of attachment to the remainder of the molecule.

In some embodiments, V is CH; Y is N; G is absent and X is connected with a nitrogen of Ring A; Ring A is

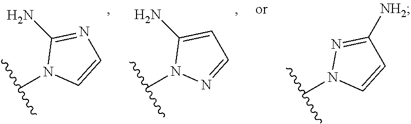 [SMILES: *n1ccnc1N ; *n1nccc1N ; *n1ccc(N)n1]

R¹ is ethyl, isopropyl, —CH₂—CHF₂,

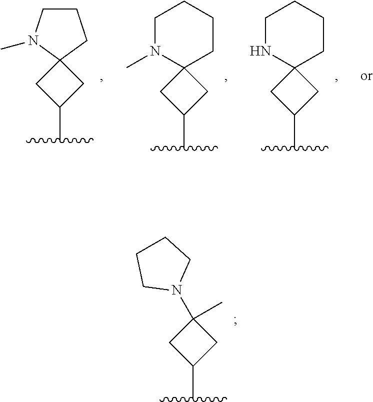

R² is isopropyl; and the spiro moiety represented by the below formula:

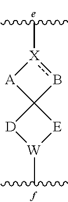

is

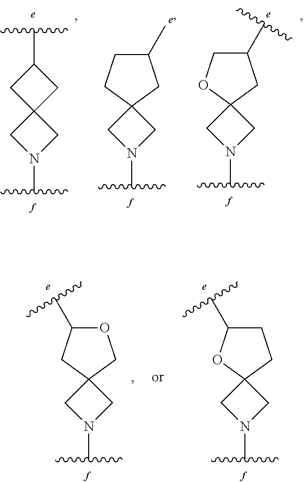

wherein e and f indicate points of attachment to the remainder of the molecule.

In some embodiments, V is CH; Y is N; G is absent and X is connected with a nitrogen of Ring A; Ring A is

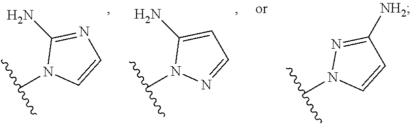 [SMILES: *n1ccnc1N ; *n1nccc1N ; *n1ccc(N)n1]

R¹ is ethyl, isopropyl, —CH₂—CHF₂, or

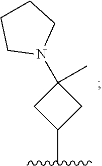 [SMILES: *C1CC(C)(N2CCCC2)C1]

R² is isopropyl; and the spiro moiety represented by the below formula:

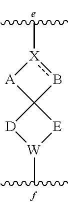

is

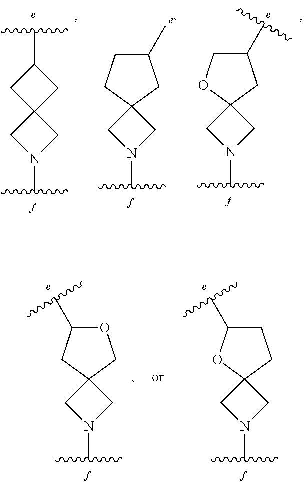

wherein e and f indicate points of attachment to the remainder of the molecule.

In some embodiments, V is CH; Y is N; G is absent and X is connected with a nitrogen of Ring A; R¹ and R² form

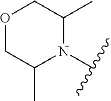 [SMILES: *N1C(C)COCC1C]

group with the nitrogen to which they are connected; and the spiro moiety represented by the below formula:

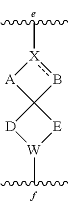

is

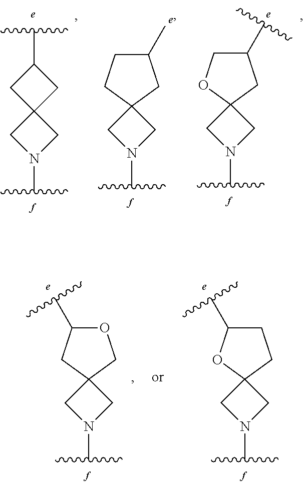

wherein e and f indicate points of attachment to the remainder of the molecule.

In some embodiments, V is CH; Y is N; G is absent and X is connected with a nitrogen of Ring A; Ring A is

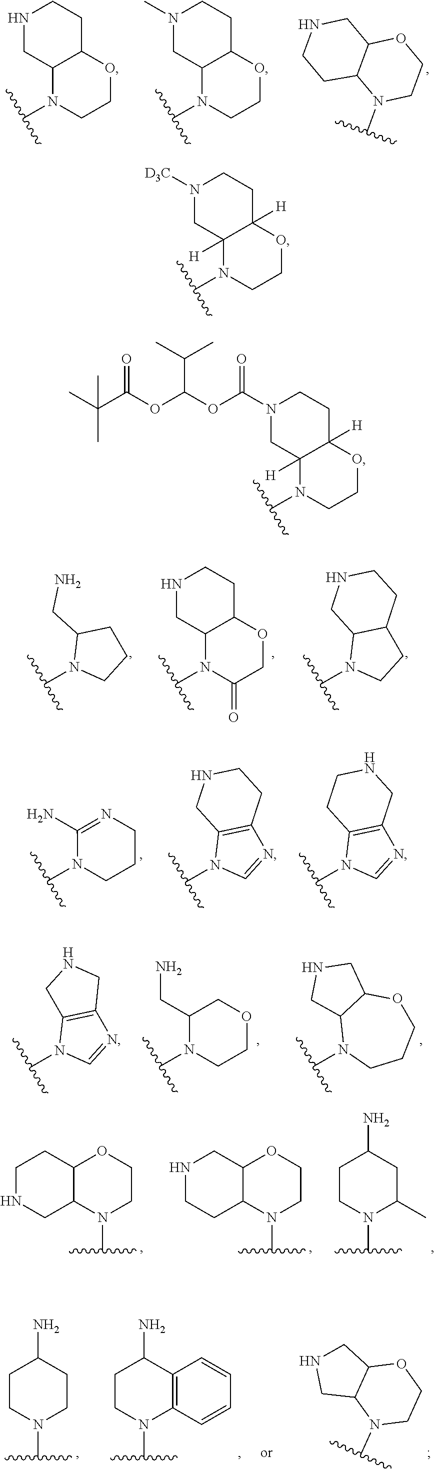

R¹ and R² form

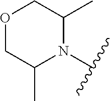 [SMILES: *N1C(C)COCC1C]

group with the nitrogen to which they are connected; and the spiro moiety represented by the below formula:

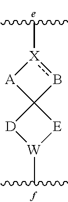

is

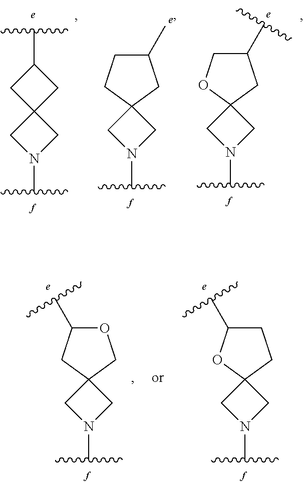

wherein e and f indicate points of attachment to the remainder of the molecule.

In some embodiments, V is CH; Y is N; G is absent and X is connected with a nitrogen of Ring A; Ring A is

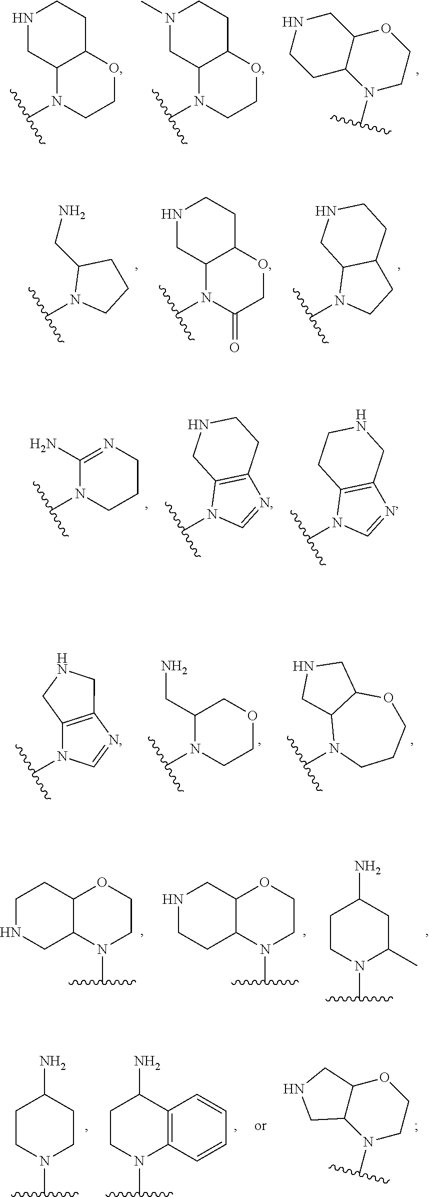

R¹ and R² form

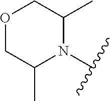 [SMILES: *N1C(C)COCC1C]

group with the nitrogen to which they are connected; and the spiro moiety represented by the below formula:

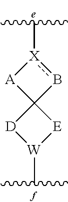

is

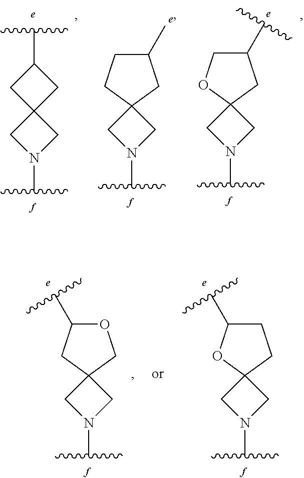

wherein e and f indicate points of attachment to the remainder of the molecule.

In some embodiments, V is CH; Y is N; G is absent and X is connected with a nitrogen of Ring A; Ring A is

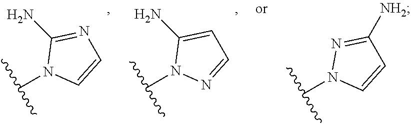 [SMILES: *n1ccnc1N ; *n1nccc1N ; *n1ccc(N)n1]

R¹ and R² form

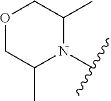 [SMILES: *N1C(C)COCC1C]

group with the nitrogen to which they are connected; and the spiro moiety represented by the below formula:

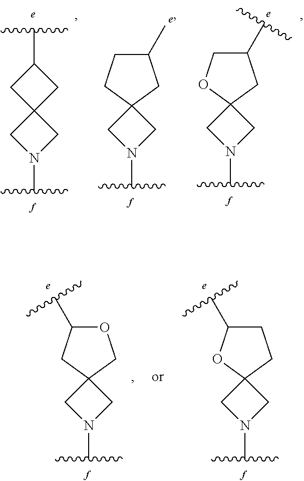

is

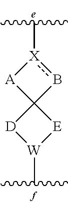

wherein e and f indicate points of attachment to the remainder of the molecule.

In some embodiments, V is N, Y is N, and G is CH₂ and G is connected with the nitrogen of Ring A.

In some embodiments, V is N; Y is N; G is CH₂ and G is connected with the nitrogen of Ring A; and Ring A is

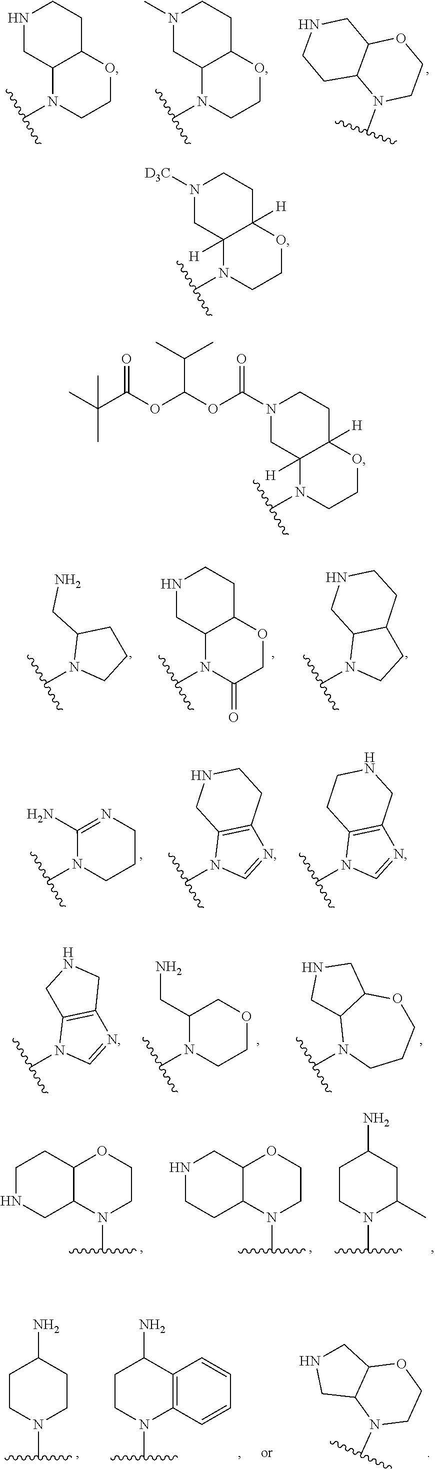

In some embodiments, V is N; Y is N; G is CH₂ and G is connected with the nitrogen of Ring A; and Ring A is

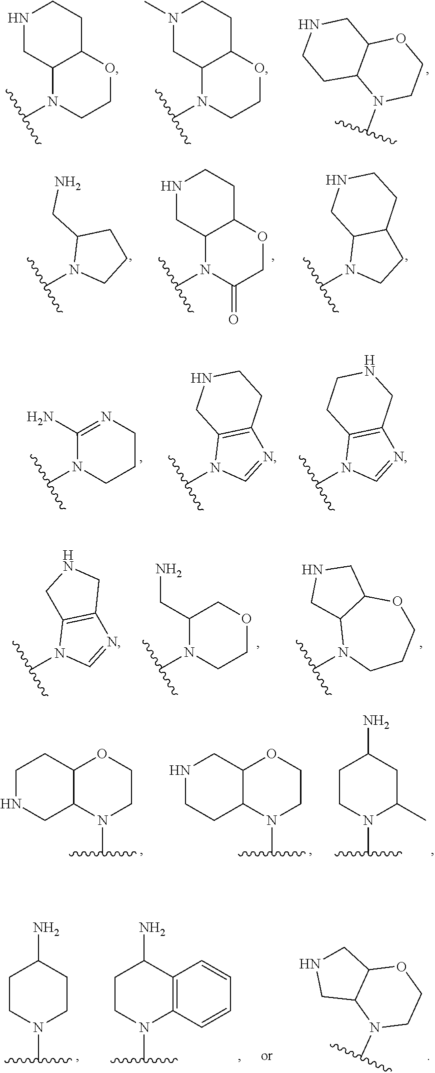

In some embodiments, V is N; Y is N; G is CH₂ and G is connected with the nitrogen of Ring A; and Ring A is

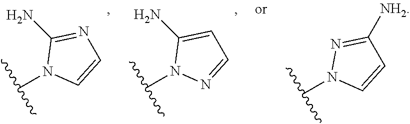 [SMILES: *n1ccnc1N ; *n1nccc1N ; *n1ccc(N)n1]

In some embodiments, V is N, Y is N, G is CH₂ and G is connected with the nitrogen of Ring A, and R² is isopropyl.

In some embodiments, V is N; Y is N; G is CH₂ and G is connected with the nitrogen of Ring A; Ring A is

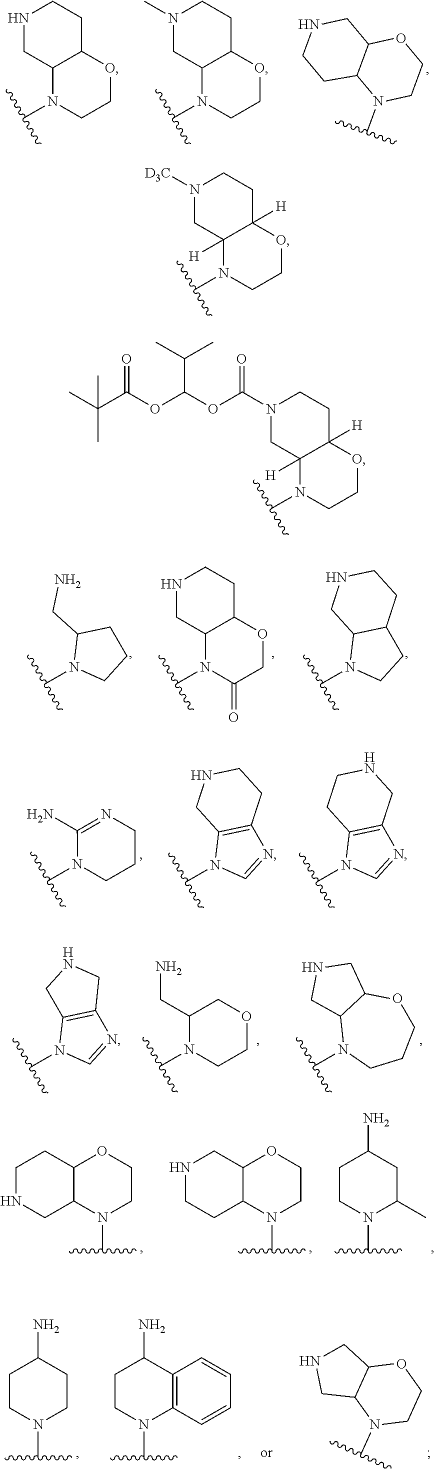

and R² is isopropyl.

In some embodiments, V is N; Y is N; G is CH₂ and G is connected with the nitrogen of Ring A; Ring A is

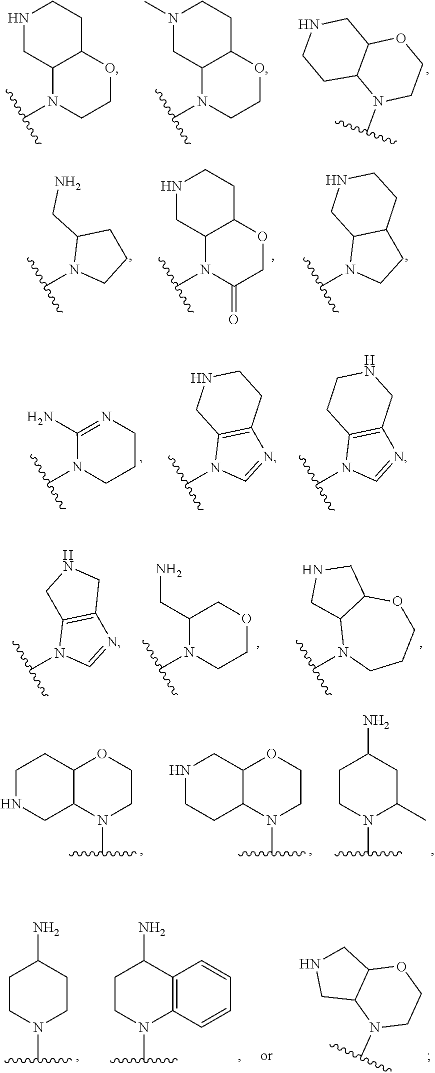

and R² is isopropyl.

In some embodiments, V is N; Y is N; G is CH₂ and G is connected with the nitrogen of Ring A; Ring A is

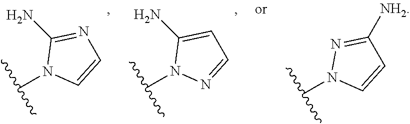 [SMILES: *n1ccnc1N ; *n1nccc1N ; *n1ccc(N)n1]

and R² is isopropyl.

In some embodiments, V is N; Y is N; G is CH₂ and G is connected with the nitrogen of Ring A; and R¹ is ethyl, isopropyl, —CH₂—CHF₂,

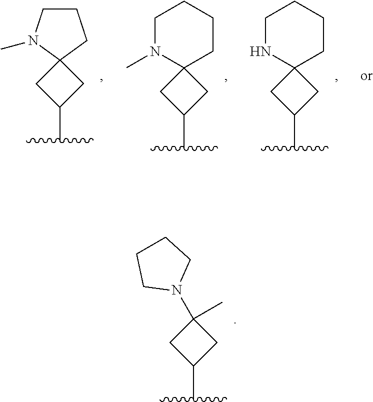

In some embodiments, V is N; Y is N; G is CH₂ and G is connected with the nitrogen of Ring A; and R¹ is ethyl, isopropyl, —CH₂—CHF₂, or

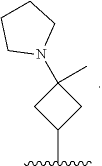 [SMILES: *C1CC(C)(N2CCCC2)C1]

In some embodiments, V is N; Y is N; G is CH₂ and G is connected with the nitrogen of Ring A; Ring A is

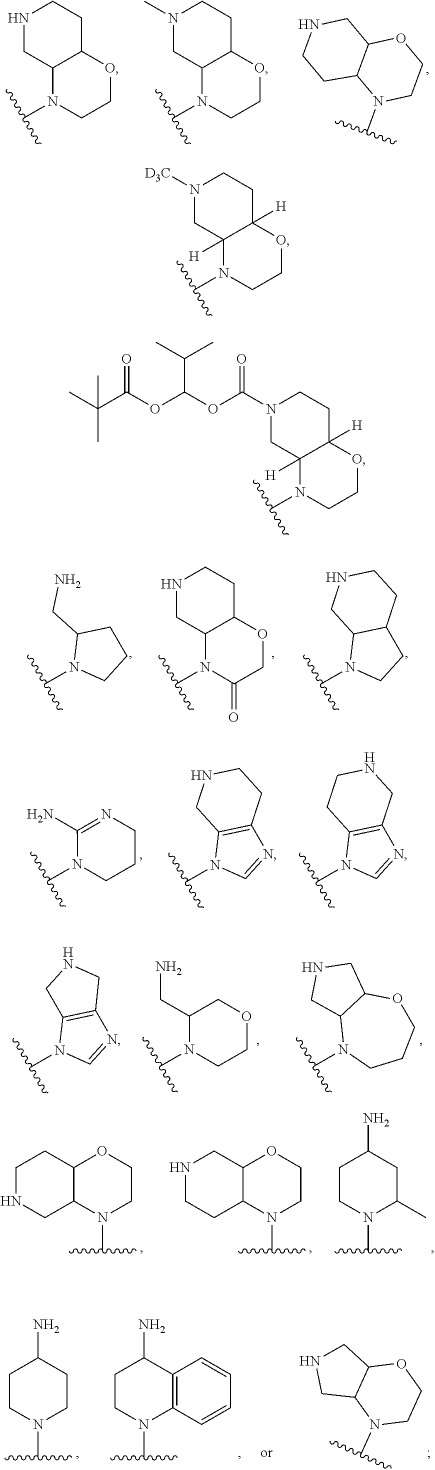

and R¹ is ethyl, isopropyl, —CH₂—CHF₂,

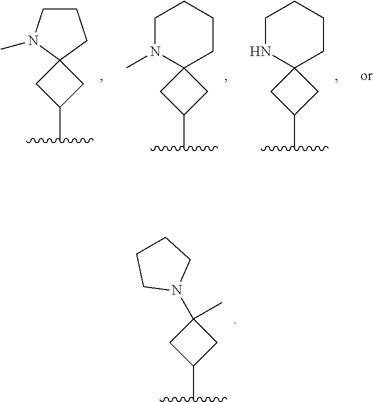

In some embodiments, V is N; Y is N; G is CH₂ and G is connected with the nitrogen of Ring A; Ring A is

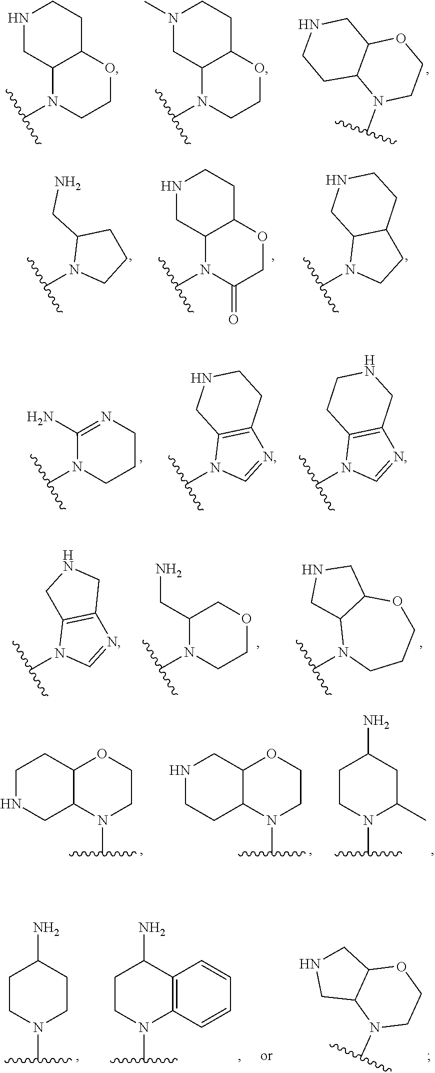

and R¹ is ethyl, isopropyl, —CH₂—CHF₂, or

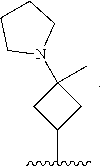 [SMILES: *C1CC(C)(N2CCCC2)C1]

In some embodiments V is N; Y is N; G is CH₂ and G is connected with the nitrogen of Ring A; Ring A is

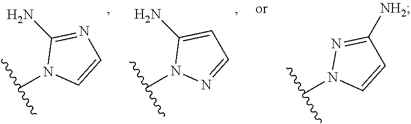 [SMILES: *n1ccnc1N ; *n1nccc1N ; *n1ccc(N)n1]

and R¹ is ethyl, isopropyl, —CH₂—CHF₂

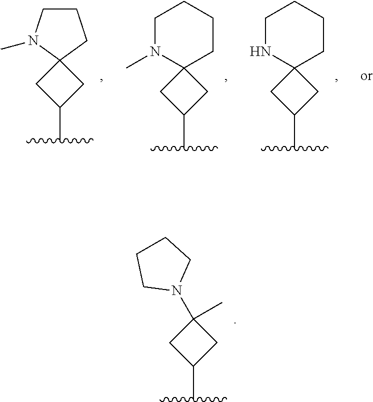

In some embodiments, V is N, Y is N, G is CH₂ and G is connected with the nitrogen of

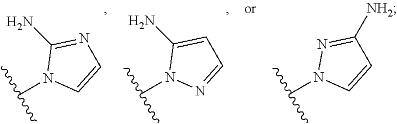 [SMILES: *n1ccnc1N ; *n1nccc1N ; *n1ccc(N)n1]

and R¹ is ethyl, isopropyl, —CH₂—CHF₂, or

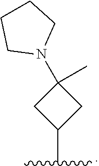 [SMILES: *C1CC(C)(N2CCCC2)C1]

Ring A; R¹ is ethyl, isopropyl, —CH₂—CHF₂,

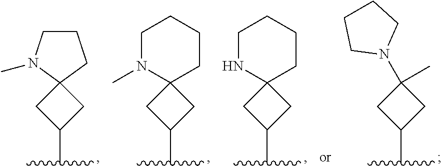

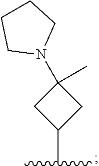 [SMILES: *C1CC(C)(N2CCCC2)C1]

and R² is isopropyl.

In some embodiments, V is N; Y is N; G is CH₂ and G is connected with the nitrogen of Ring A; Ring A is

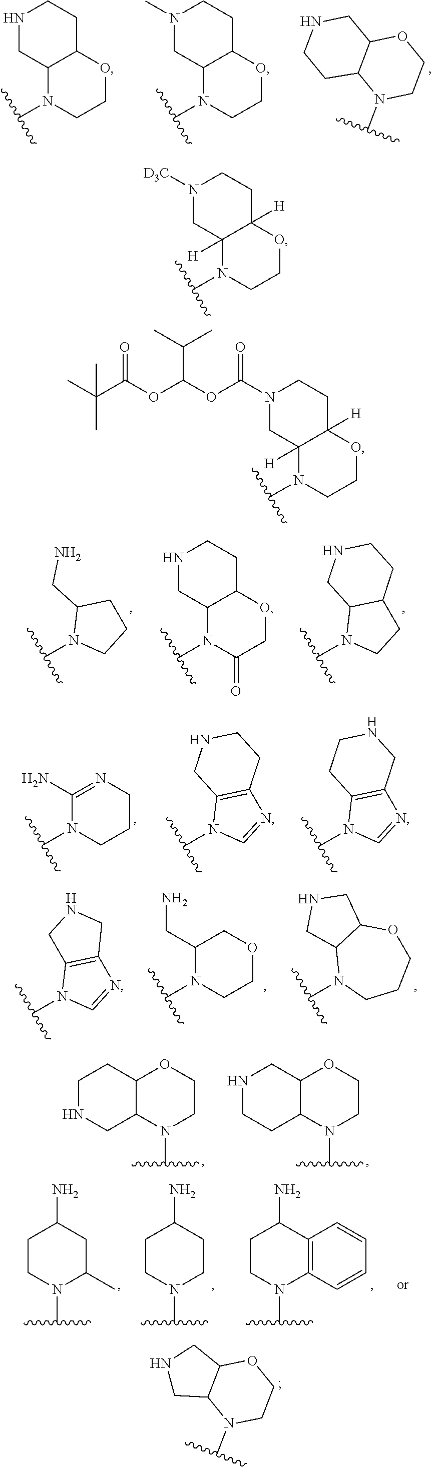

R¹ is ethyl, isopropyl, —CH₂—CHF₂,

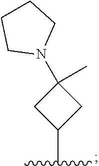 [SMILES: *C1CC(C)(N2CCCC2)C1]

and R² is isopropyl.

In some embodiments, V is N; Y is N; G is CH₂ and G is connected with the nitrogen of Ring A; Ring A is

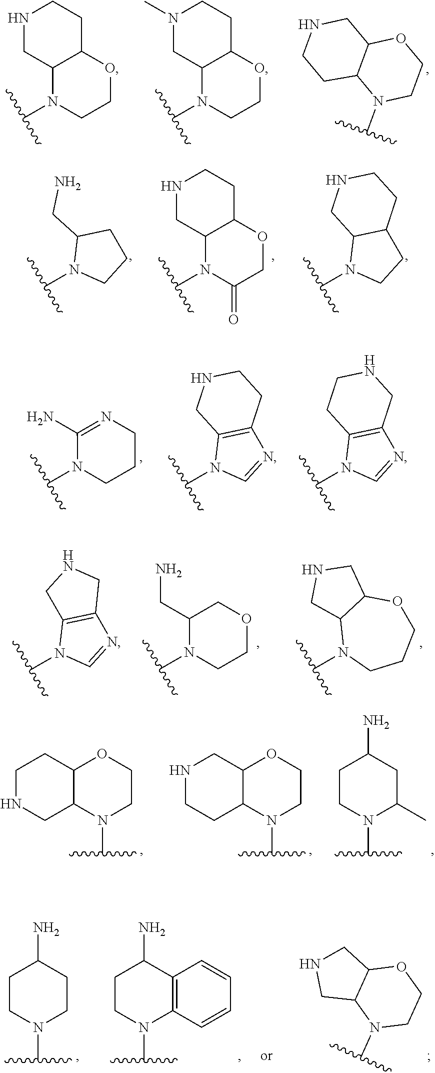

R¹ is ethyl, isopropyl, —CH₂—CHF₂, or; and R² is isopropyl.

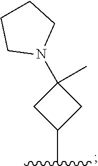 [SMILES: *C1CC(C)(N2CCCC2)C1]

In some embodiments, V is N; Y is N; G is CH₂ and G is connected with the nitrogen of Ring A; Ring A is

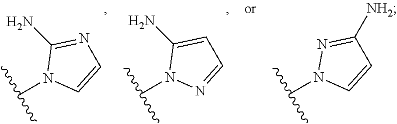 [SMILES: *n1ccnc1N ; *n1nccc1N ; *n1ccc(N)n1]

R¹ is ethyl, isopropyl, —CH₂—CHF₂,

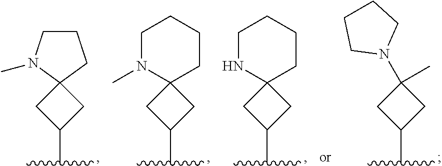

and R² is isopropyl.

In some embodiments, V is N; Y is N; G is CH₂ and G is connected with the nitrogen of Ring A; Ring A is

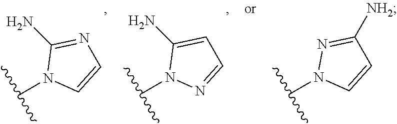 [SMILES: *n1ccnc1N ; *n1nccc1N ; *n1ccc(N)n1]

R¹ is ethyl, isopropyl, —CH₂—CHF₂,

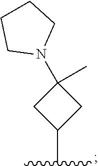 [SMILES: *C1CC(C)(N2CCCC2)C1]

and R² is isopropyl.

In some embodiments, V is N; Y is N; G is CH₂ and G is connected with the nitrogen of Ring A, R¹ and R² form

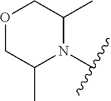 [SMILES: *N1C(C)COCC1C]

group with the nitrogen to which they are connected.

In some embodiments, V is N; Y is N; G is CH₂ and G is connected with the nitrogen of Ring A; Ring A is

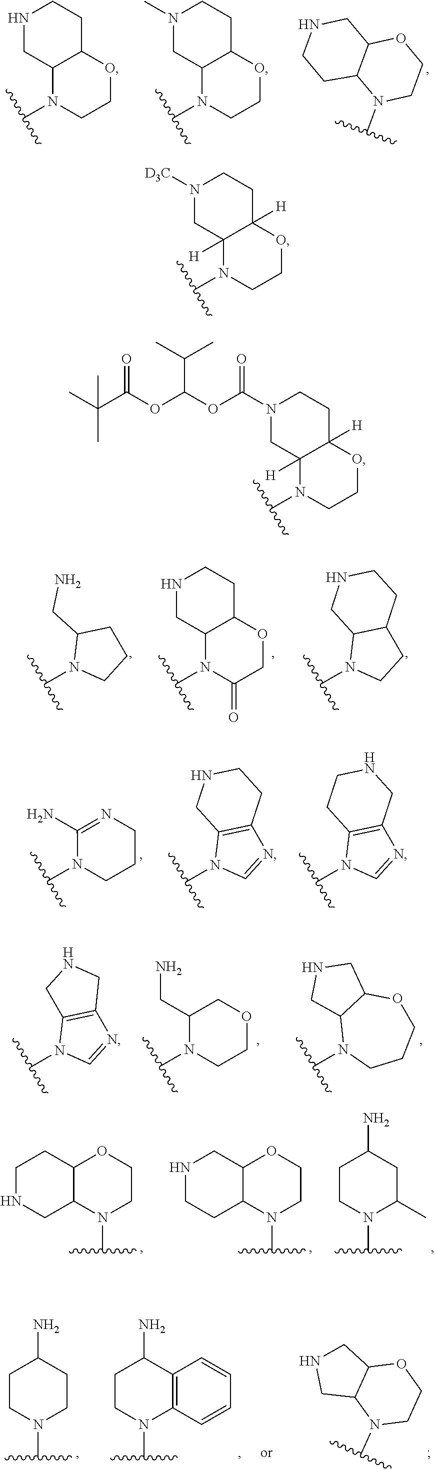

and R¹ and R² form

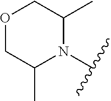 [SMILES: *N1C(C)COCC1C]

group with the nitrogen to which they are connected.

In some embodiments, V is N; Y is N; G is CH₂ and G is connected with the nitrogen of Ring A; Ring A is

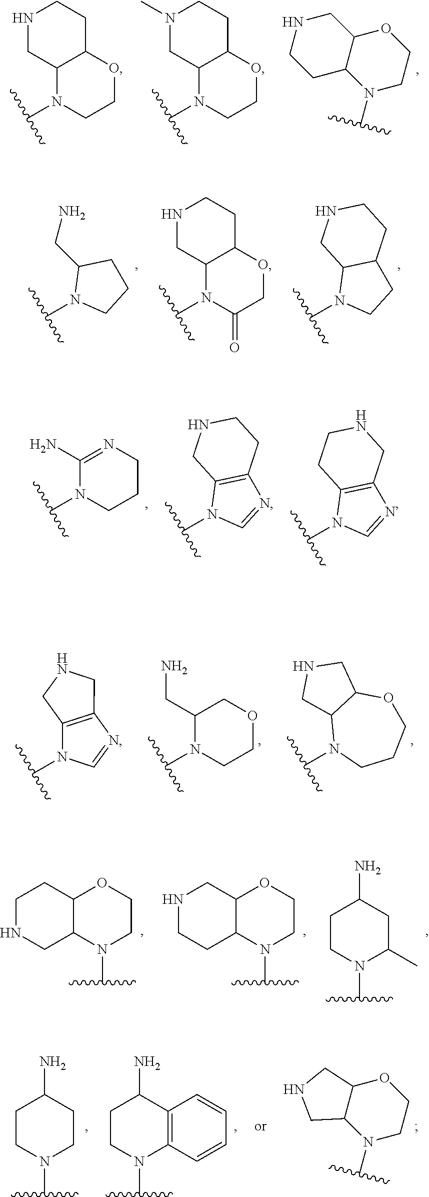

and R¹ and R² form

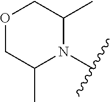 [SMILES: *N1C(C)COCC1C]

group with the nitrogen to which they are connected.

In some embodiments, V is N; Y is N; G is CH₂ and G is connected with the nitrogen of Ring A; Ring A is

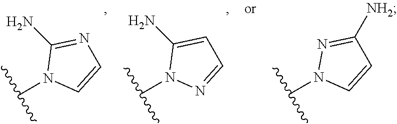 [SMILES: *n1ccnc1N ; *n1nccc1N ; *n1ccc(N)n1]

and R¹ and R² form

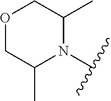 [SMILES: *N1C(C)COCC1C]

group with the nitrogen to which they are connected.

In some embodiments, V is N; Y is N; G is CH₂ and G is connected with the nitrogen of Ring A; and the spiro moiety represented by the below formula:

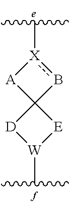

is

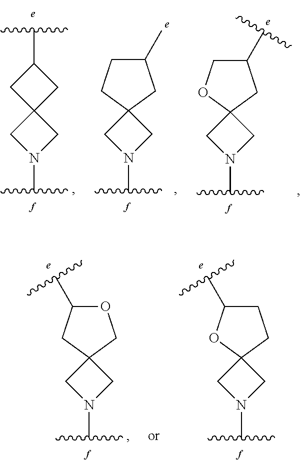

In some embodiments, V is N; Y is N; G is CH₂ and G is connected with the nitrogen of Ring A; Ring A is

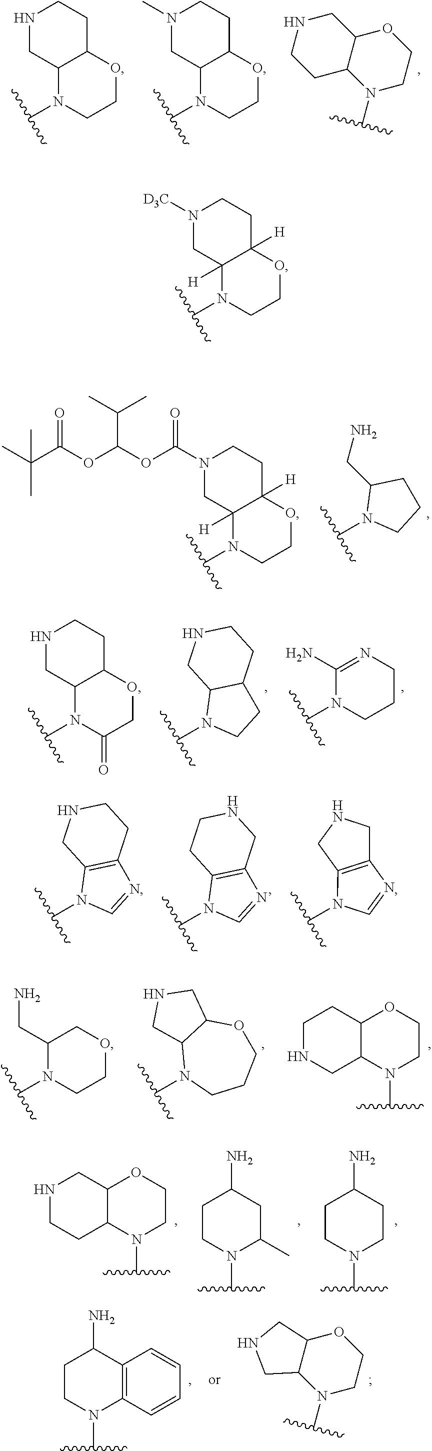

and the spiro moiety represented by the below formula:

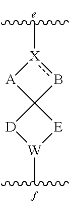

is

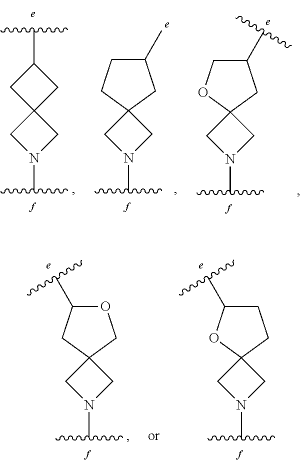

wherein e and f indicate points of attachment to the remainder of the molecule.

In some embodiments, V is N; Y is N; G is CH₂ and G is connected with the nitrogen of Ring A; Ring A is

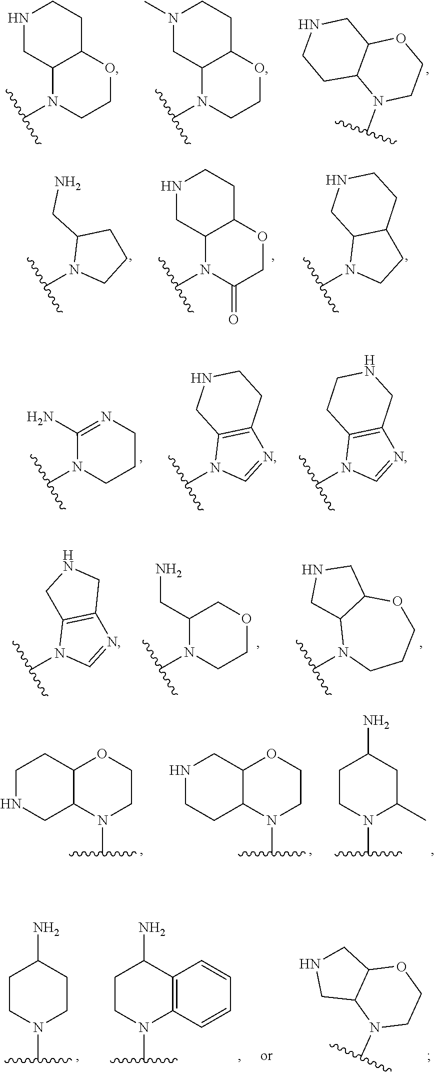

and the spiro moiety represented by the below formula:

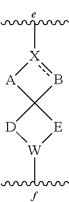

is

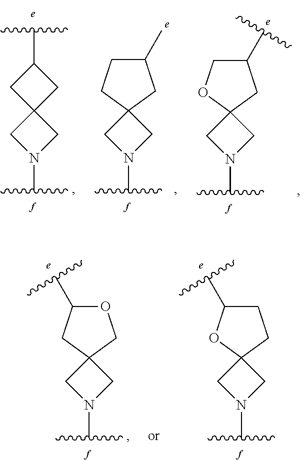

or wherein e and f indicate points of attachment to the remainder of the molecule.

In some embodiments, V is N; Y is N; G is CH₂ and G is connected with the nitrogen of Ring A; Ring A is

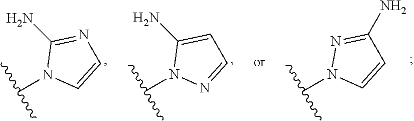 [SMILES: *n1ccnc1N ; *n1nccc1N ; *n1ccc(N)n1]

and the spiro moiety represented by the below formula:

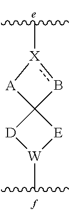

is

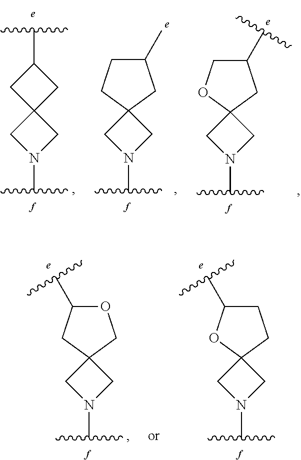

wherein e and f indicate points of attachment to the remainder of the molecule.

In some embodiments, V is N, Y is N, G is CH₂ and G is connected with the nitrogen of Ring A, R² is isopropyl; and the spiro moiety represented by the below formula:

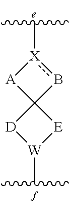

is

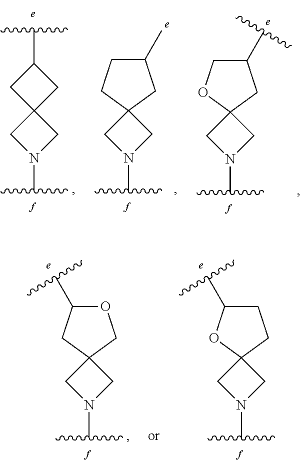

or wherein e and f indicate points of attachment to the remainder of the molecule.

In some embodiments, V is N; Y is N; G is CH₂ and G is connected with the nitrogen of Ring A; Ring A is

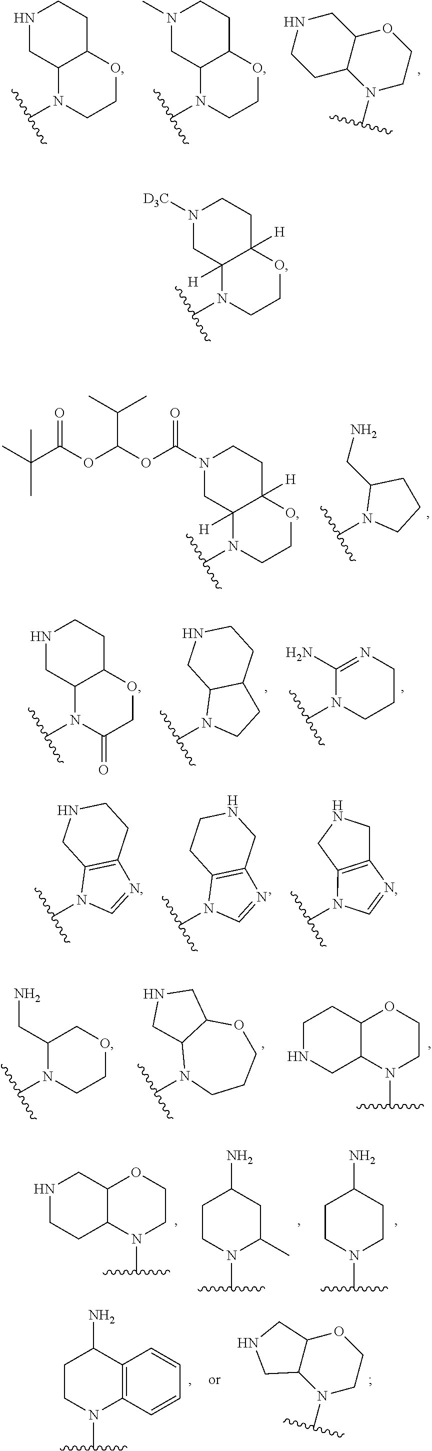

R² is isopropyl; and the spiro moiety represented by the below formula:

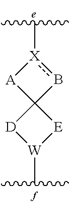

is

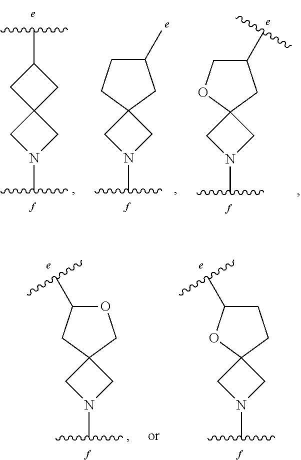

wherein e and f indicate points of attachment to the remainder of the molecule.

In some embodiments, V is N; Y is N; G is CH₂ and G is connected with the nitrogen of Ring A; Ring A is

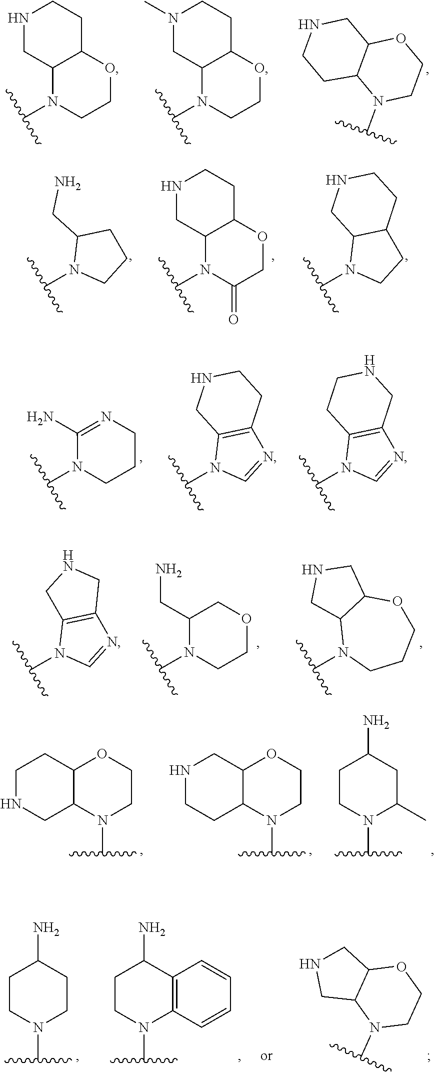

R² is isopropyl; and the spiro moiety represented by the below formula:

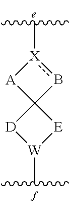

is

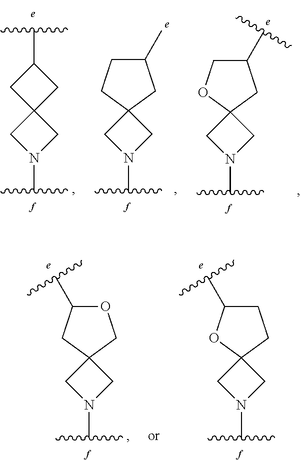

wherein e and f indicate points of attachment to the remainder of the molecule.

In some embodiments, V is N; Y is N; G is CH₂ and G is connected with the nitrogen of Ring A; Ring A is

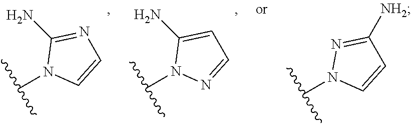 [SMILES: *n1ccnc1N ; *n1nccc1N ; *n1ccc(N)n1]

R² is isopropyl; and the spiro moiety represented by the below formula:

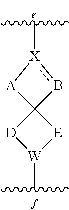

is

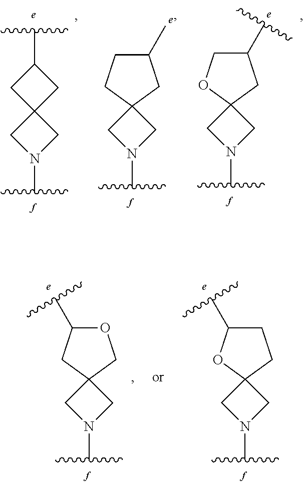

wherein e and f indicate points of attachment to the remainder of the molecule.

In some embodiments, V is N; Y is N; G is CH₂ and G is connected with the nitrogen of Ring A; R¹ is ethyl, isopropyl, —CH₂—CHF₂,

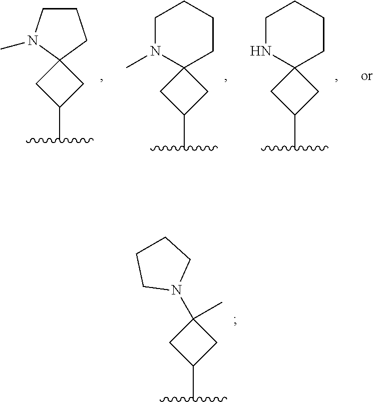

and the spiro moiety represented by the below formula:

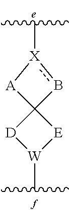

is

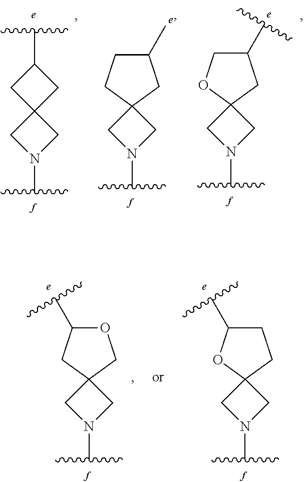

wherein e and f indicate points of attachment to the remainder of the molecule.

In some embodiments, V is N; Y is N; G is CH₂ and G is connected with the nitrogen of Ring A; R¹ is ethyl, isopropyl, —CH₂—CHF₂,

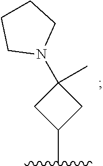 [SMILES: *C1CC(C)(N2CCCC2)C1]

and the spiro moiety represented by the below formula:

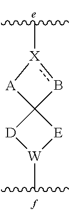

is

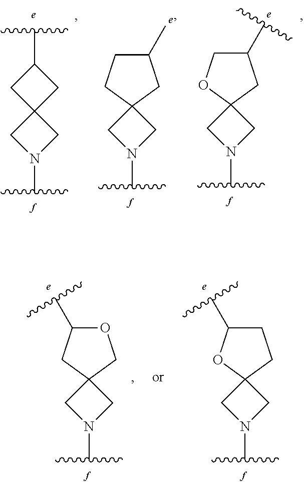

wherein e and f indicate points of attachment to the remainder of the molecule.

In some embodiments, V is N; Y is N; G is CH₂ and G is connected with the nitrogen of Ring A; Ring A is

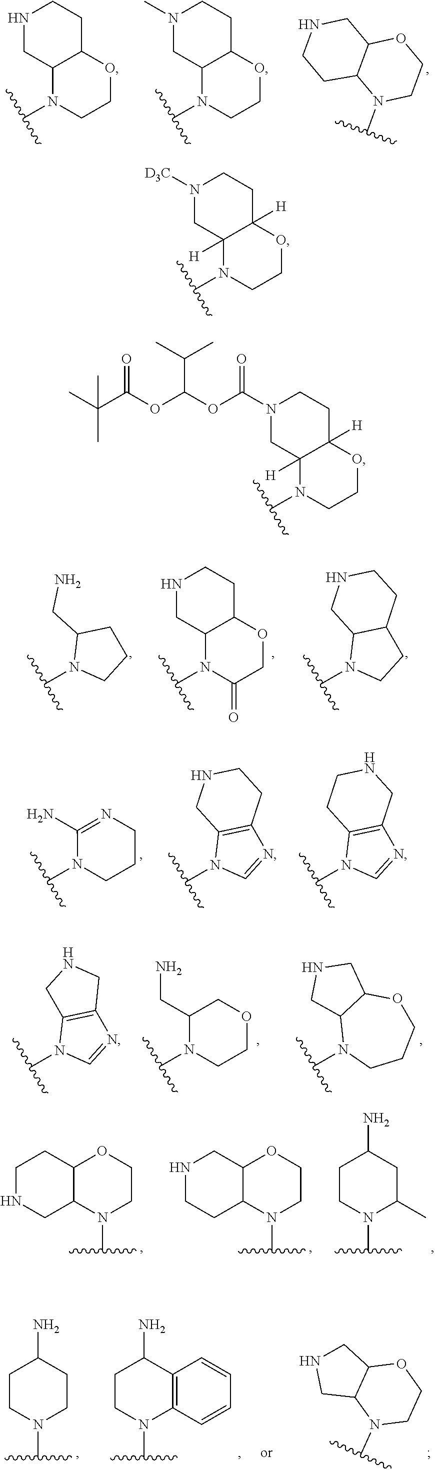

R¹ is ethyl, isopropyl, —CH₂—CHF₂,

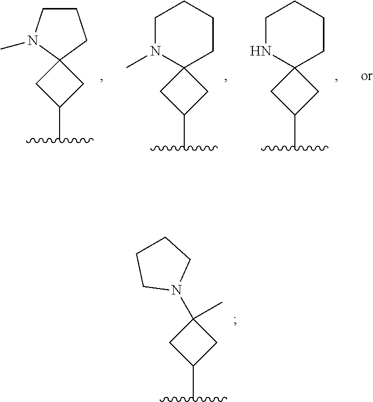

and the spiro moiety represented by the below formula:

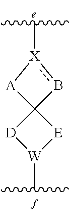

is

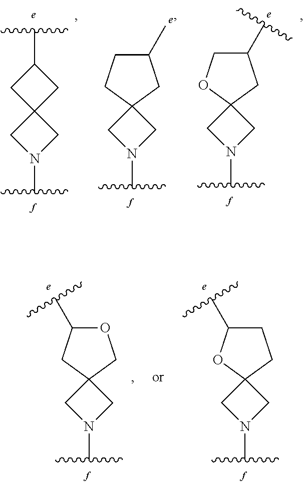

wherein e and f indicate points of attachment to the remainder of the molecule.

In some embodiments, V is N; Y is N; G is CH₂ and G is connected with the nitrogen of Ring A; Ring A is

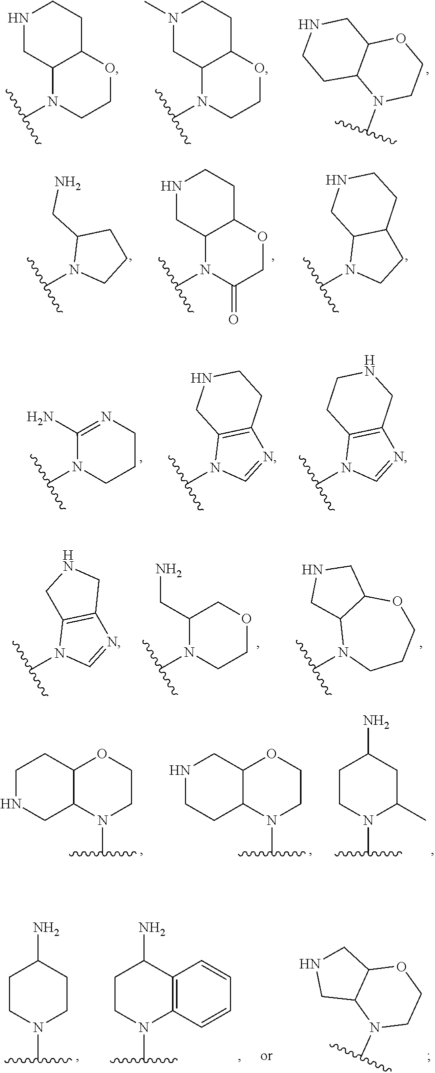

R¹ is ethyl, isopropyl, —CH₂—CHF₂, or

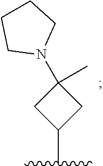 [SMILES: *C1CC(C)(N2CCCC2)C1]

and the spiro moiety represented by the below formula:

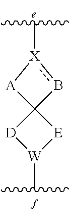

is

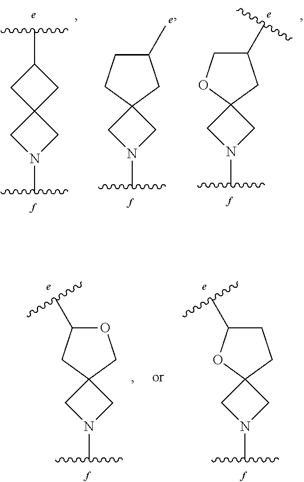

wherein e and f indicate points of attachment to the remainder of the molecule.

In some embodiments, V is N; Y is N; G is CH₂ and G is connected with the nitrogen of Ring A; Ring A is

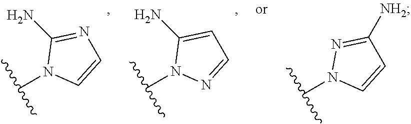 [SMILES: *n1ccnc1N ; *n1nccc1N ; *n1ccc(N)n1]

R¹ is ethyl, isopropyl, —CH₂—CHF₂,

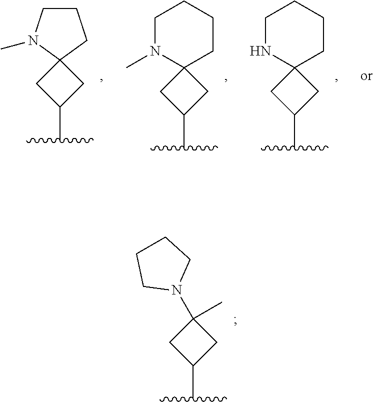

and the spiro moiety represented by the below formula:

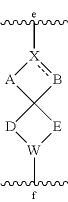

is

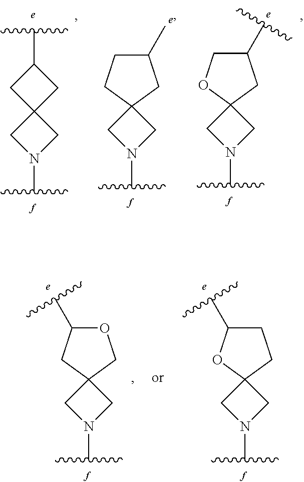

wherein e and f indicate points of attachment to the remainder of the molecule.

In some embodiments, V is N; Y is N; G is CH₂ and G is connected with the nitrogen of Ring A; Ring A is

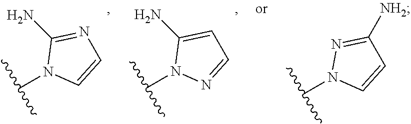 [SMILES: *n1ccnc1N ; *n1nccc1N ; *n1ccc(N)n1]

R¹ is ethyl, isopropyl, —CH₂—CHF₂,

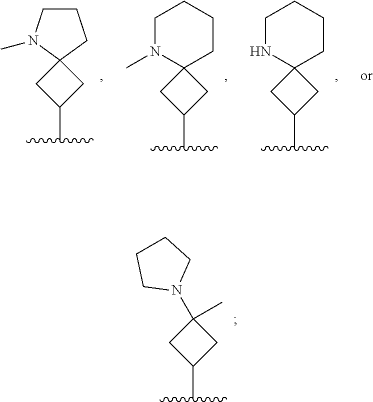

and the spiro moiety represented by the below formula:

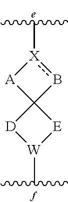

is

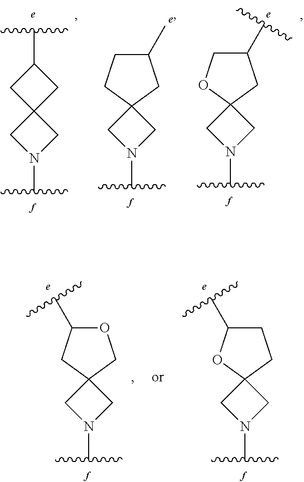

wherein e and f indicate points of attachment to the remainder of the molecule.

In some embodiments, V is N; Y is N; G is CH₂ and G is connected with the nitrogen of Ring A; Ring A is

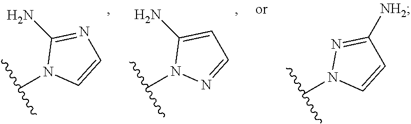 [SMILES: *n1ccnc1N ; *n1nccc1N ; *n1ccc(N)n1]

R¹ is ethyl, isopropyl, —CH₂—CHF₂, or

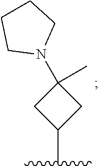 [SMILES: *C1CC(C)(N2CCCC2)C1]

and the spiro moiety represented by the below formula:

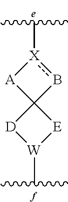

is

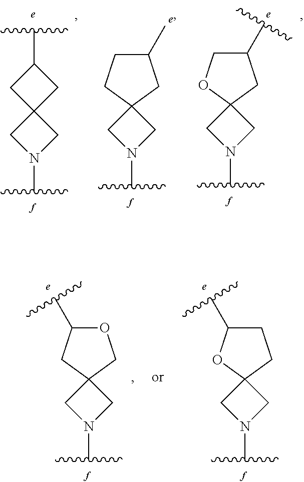

wherein e and f indicate points of attachment to the remainder of the molecule.

In some embodiments, V is N, Y is N, G is CH₂ and G is connected with the nitrogen of Ring A; R¹ is ethyl, isopropyl, —CH₂—CHF₂,

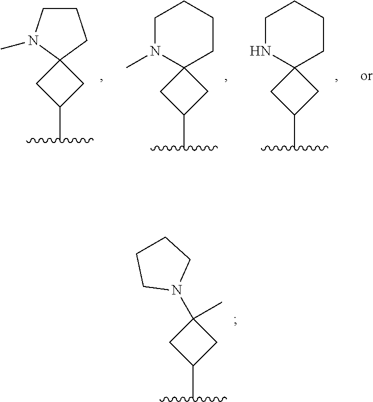

R² is isopropyl; and the spiro moiety represented by the below formula:

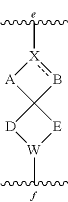

is

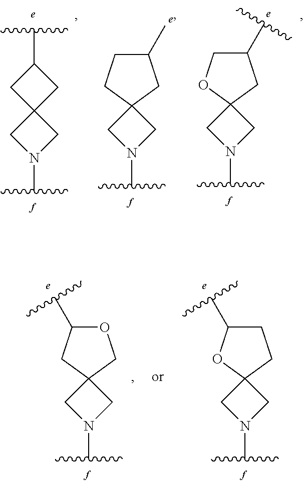

wherein e and f indicate points of attachment to the remainder of the molecule.

In some embodiments, V is N, Y is N, G is CH₂ and G is connected with the nitrogen of Ring A; R¹ is ethyl, isopropyl, —CH₂—CHF₂,

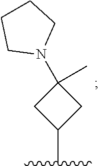 [SMILES: *C1CC(C)(N2CCCC2)C1]

R² is isopropyl; and the spiro moiety represented by the below formula:

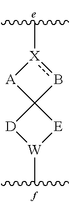

is

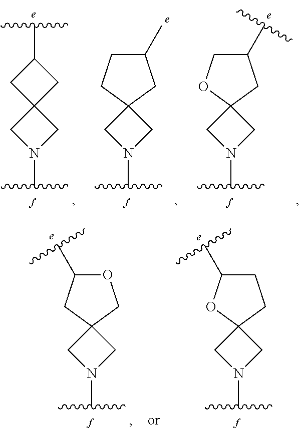

wherein e and f indicate points of attachment to the remainder of the molecule.

In some embodiments, V is N; Y is N; G is CH₂ and G is connected with the nitrogen of Ring A; Ring A is

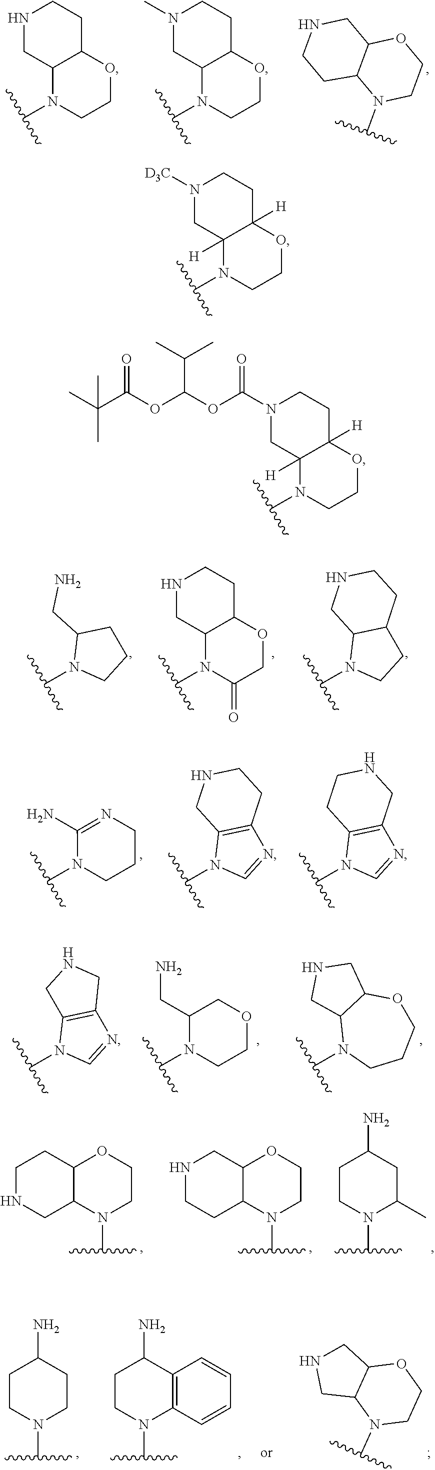

R¹ is ethyl, isopropyl, —CH₂—CHF₂,

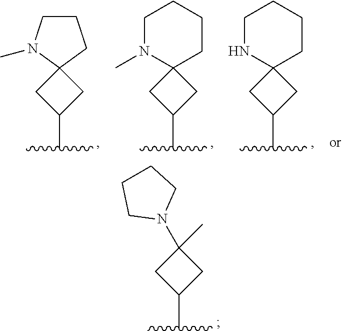

R² is isopropyl; and the spiro moiety represented by the below formula:

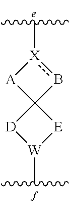

is

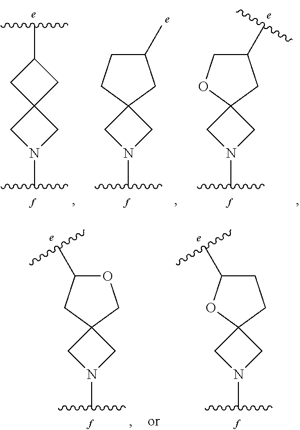

wherein e and f indicate points of attachment to the remainder of the molecule.

In some embodiments, V is N; Y is N; G is CH₂ and G is connected with the nitrogen of Ring A; Ring A is

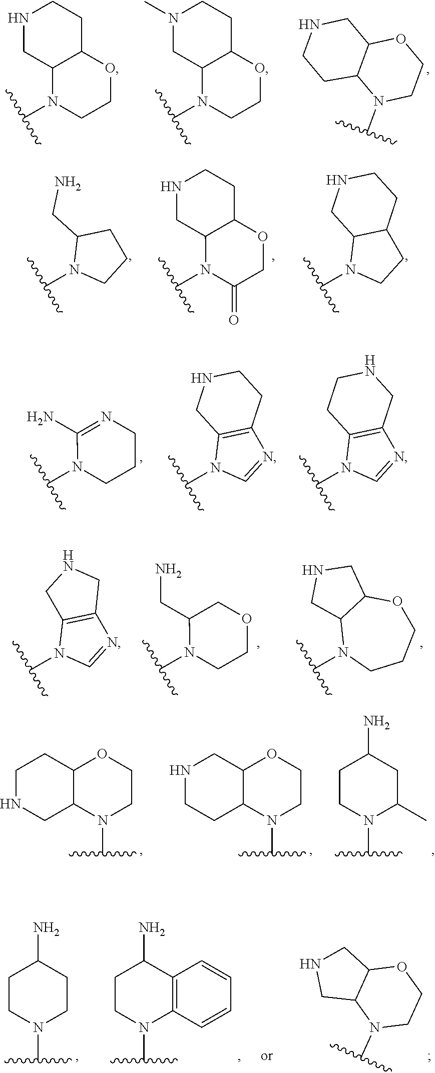

R¹ is ethyl, isopropyl, —CH₂—CHF₂, or

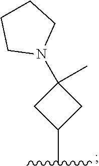 [SMILES: *C1CC(C)(N2CCCC2)C1]

R² is isopropyl; and the spiro moiety represented by the below formula:

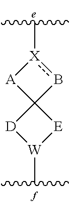

is

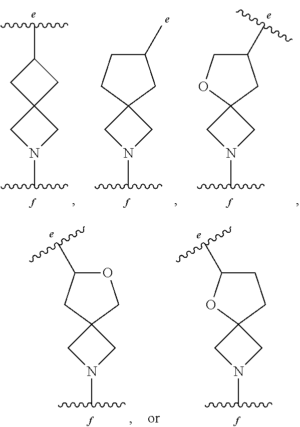

wherein e and f indicate points of attachment to the remainder of the molecule.

In some embodiments, V is N; Y is N; G is CH₂ and G is connected with the nitrogen of Ring A; Ring A is

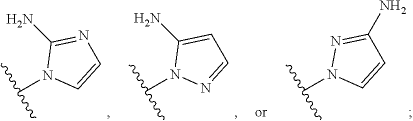 [SMILES: *n1ccnc1N ; *n1nccc1N ; *n1ccc(N)n1]

R¹ is ethyl, isopropyl, —CH₂—CHF₂,

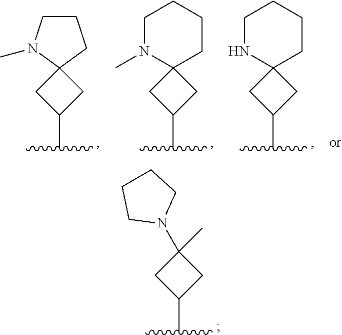

R² is isopropyl; and the spiro moiety represented by the below formula:

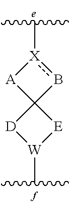

is

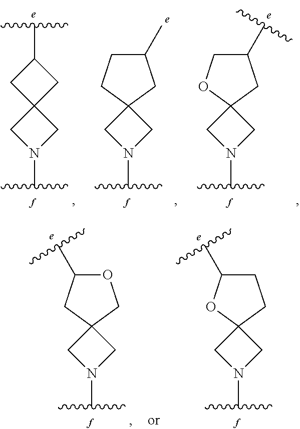

wherein e and f indicate points of attachment to the remainder of the molecule.

In some embodiments, V is N; Y is N; G is CH₂ and G is connected with the nitrogen of Ring A; Ring A is

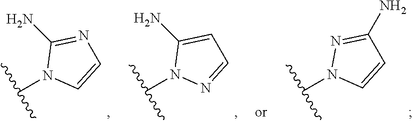 [SMILES: *n1ccnc1N ; *n1nccc1N ; *n1ccc(N)n1]

R¹ is ethyl, isopropyl, —CH₂—CHF₂, or

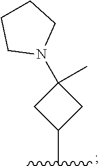 [SMILES: *C1CC(C)(N2CCCC2)C1]

R² is isopropyl; and the spiro moiety represented by the below formula:

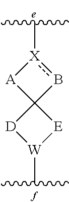

is

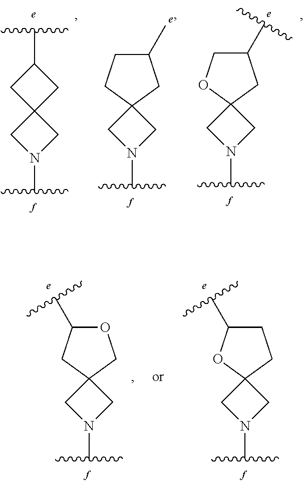

wherein e and f indicate points of attachment to the remainder of the molecule.

In some embodiments, V is N; Y is N; G is CH₂ and G is connected with the nitrogen of Ring A; R¹ and R² form

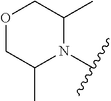 [SMILES: *N1C(C)COCC1C]

group with the nitrogen to which they are connected; and the spiro moiety represented by the below formula:

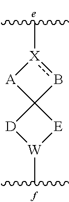

is

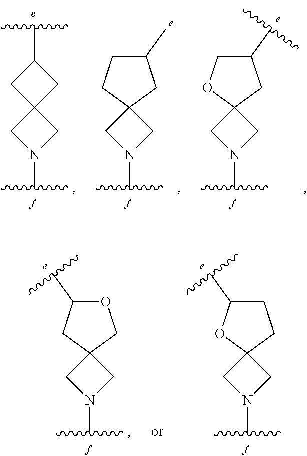

wherein e and f indicate points of attachment to the remainder of the molecule.

In some embodiments, V is N; Y is N; G is CH₂ and G is connected with the nitrogen of Ring A; Ring A is

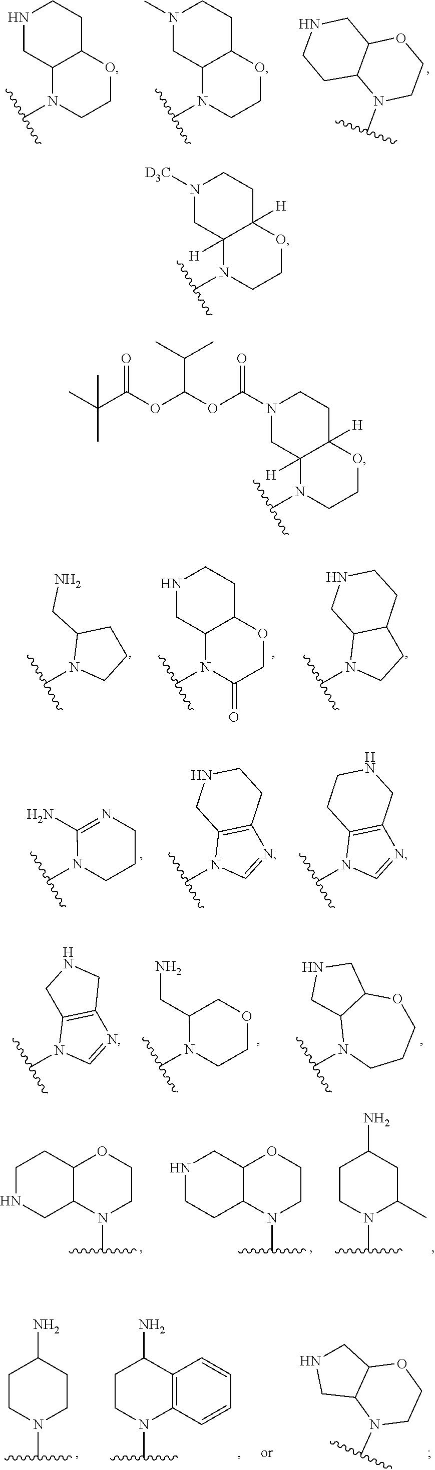

R¹ and R² form

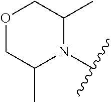 [SMILES: *N1C(C)COCC1C]

group with the nitrogen to which they are connected; and the spiro moiety represented by the below formula:

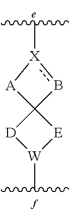

is

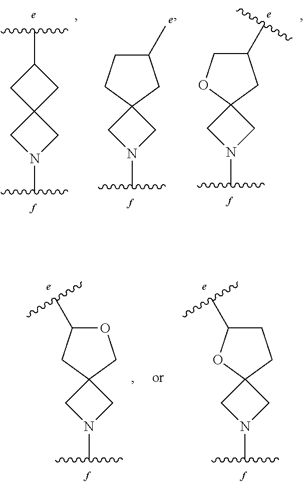

wherein e and f indicate points of attachment to the remainder of the molecule.

In some embodiments, V is N; Y is N; G is CH₂ and G is connected with the nitrogen of Ring A; Ring A is

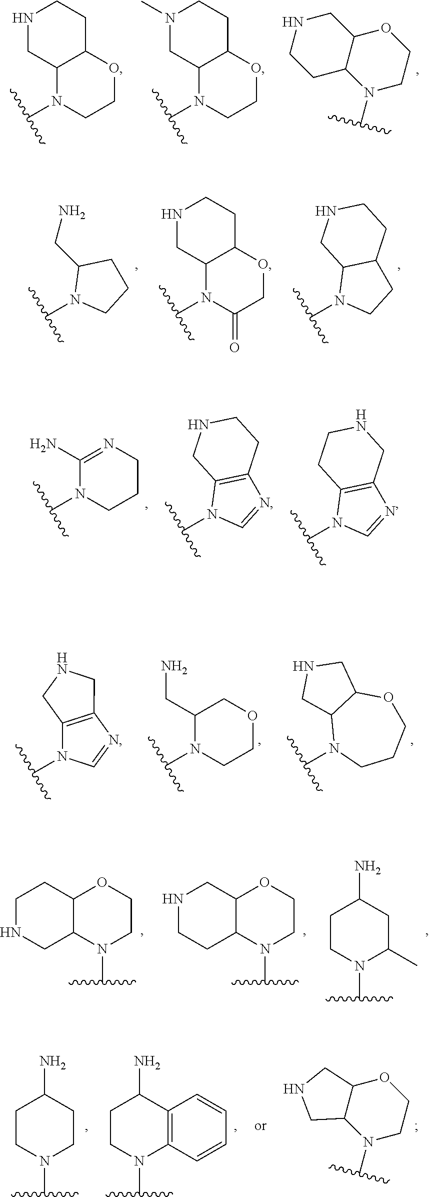

R¹ and R² form

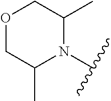 [SMILES: *N1C(C)COCC1C]

group with the nitrogen to which they are connected; and the spiro moiety represented by the below formula:

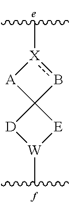

is

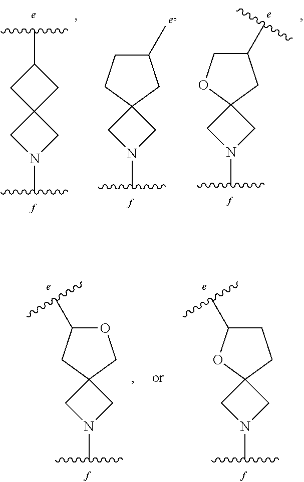

wherein e and f indicate points of attachment to the remainder of the molecule.

In some embodiments, V is N; Y is N; G is CH₂ and G is connected with the nitrogen of Ring A; Ring A is

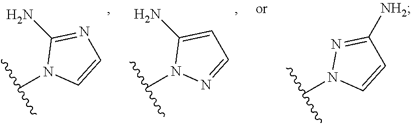 [SMILES: *n1ccnc1N ; *n1nccc1N ; *n1ccc(N)n1]

R¹ and R² form

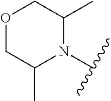 [SMILES: *N1C(C)COCC1C]

group with the nitrogen to which they are connected; and the spiro moiety represented by the below formula:

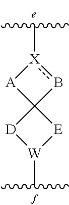

is

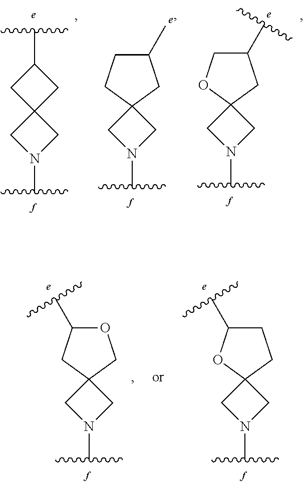

wherein e and f indicate points of attachment to the remainder of the molecule.

In some embodiments, V is N, Y is CH, and G is CH₂ and G is connected with the nitrogen of Ring A.

In some embodiments, V is N; Y is CH; G is CH₂ and G is connected with the nitrogen of Ring A; and Ring A is

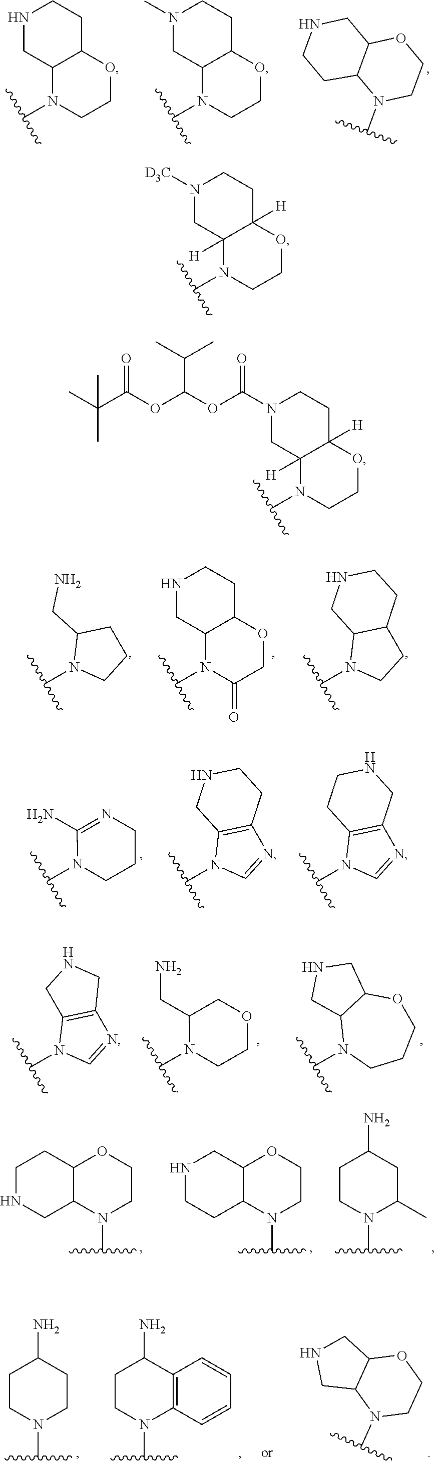

In some embodiments, V is N; Y is CH; G is CH₂ and G is connected with the nitrogen of Ring A; and Ring A is

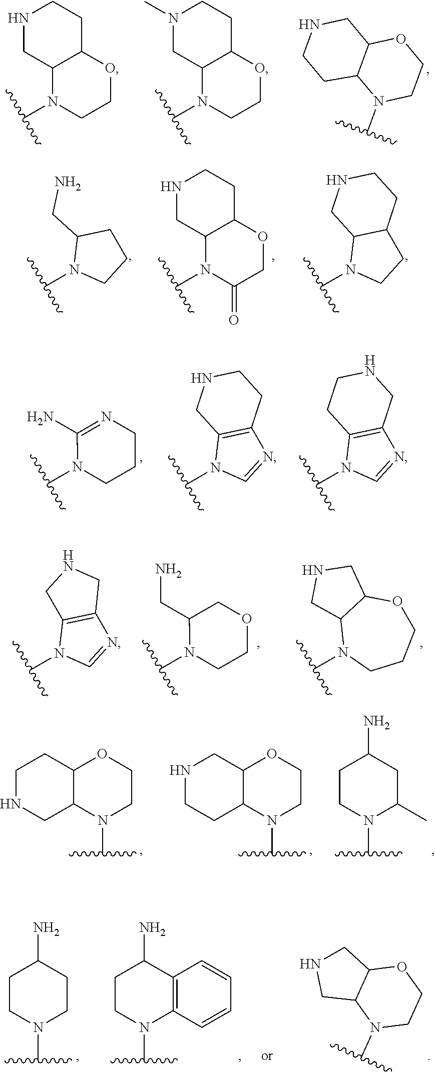

In some embodiments, V is N; Y is CH; G is CH₂ and G is connected with the nitrogen of Ring A; and Ring A is

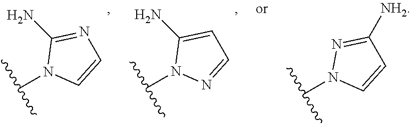 [SMILES: *n1ccnc1N ; *n1nccc1N ; *n1ccc(N)n1]

In some embodiments, V is N, Y is CH, G is CH₂ and G is connected with the nitrogen of Ring A, and R² is isopropyl.

In some embodiments, V is N; Y is CH; G is CH₂ and G is connected with the nitrogen of Ring A; Ring A is

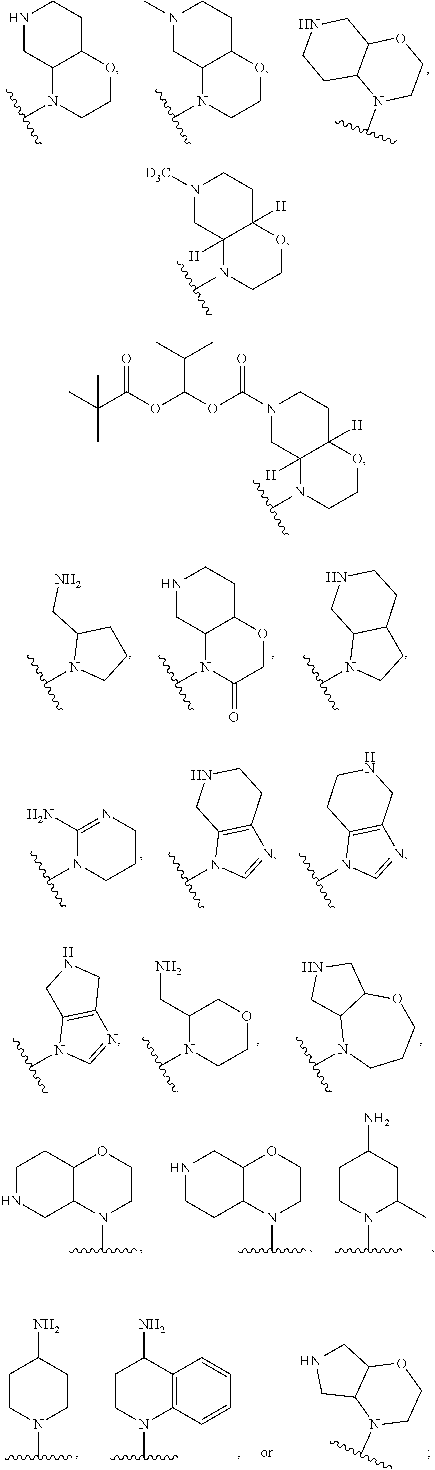

and R² is isopropyl.

In some embodiments, V is N; Y is CH; G is CH₂ and G is connected with the nitrogen of Ring A; Ring A is

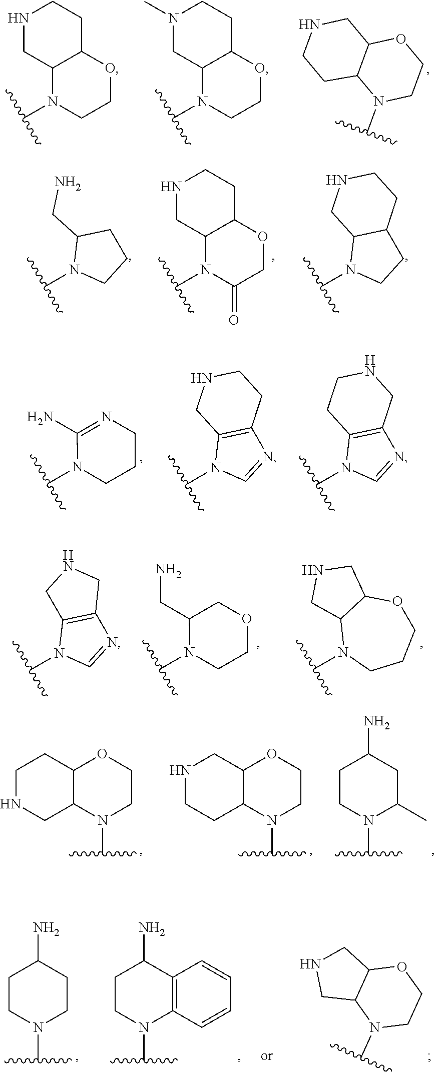

and R² is isopropyl.

In some embodiments, V is N; Y is CH; G is CH₂ and G is connected with the nitrogen of Ring A; Ring A is

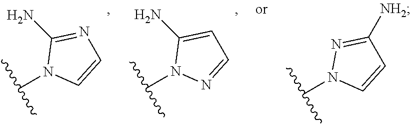 [SMILES: *n1ccnc1N ; *n1nccc1N ; *n1ccc(N)n1]

and R² is isopropyl.

In some embodiments, V is N; Y is CH; G is CH₂ and G is connected with the nitrogen of Ring A; R¹ is ethyl, isopropyl, —CH₂—CHF₂,

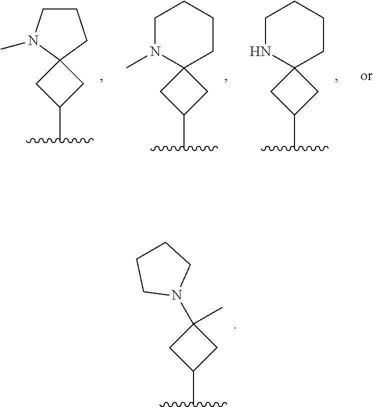

In some embodiments, V is N; Y is CH; G is CH₂ and G is connected with the nitrogen of Ring A; and R¹ is ethyl, isopropyl, —CH₂—CHF₂, or

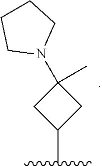 [SMILES: *C1CC(C)(N2CCCC2)C1]

In some embodiments, V is N; Y is CH; G is CH₂ and G is connected with the nitrogen of Ring A; Ring A is

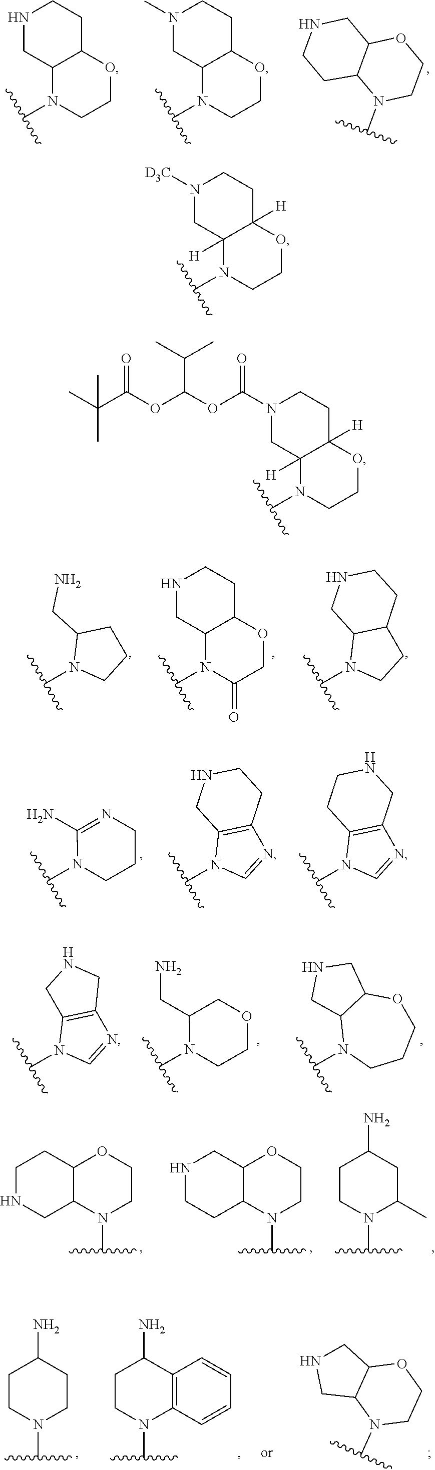

and R¹ is ethyl, isopropyl, —CH₂—CHF₂—CHF₂,

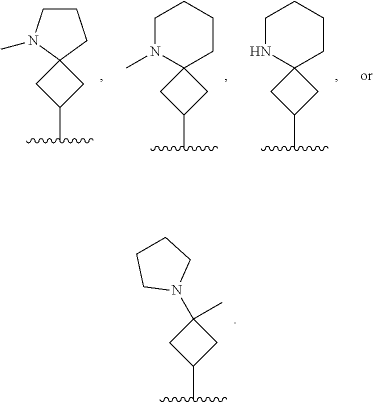

In some embodiments, V is N; Y is CH; G is CH₂ and G is connected with the nitrogen of Ring A; Ring A is

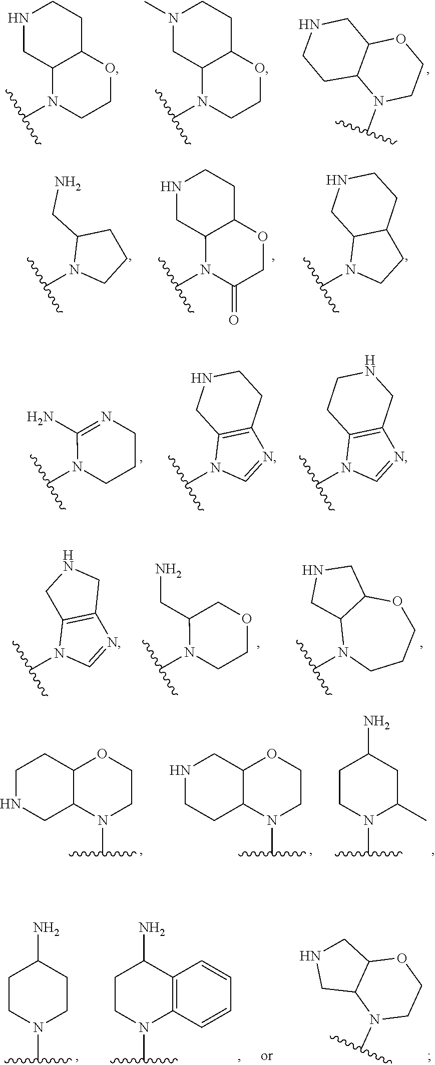

and R¹ is ethyl, isopropyl, —CH₂—CHF₂ or

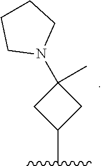 [SMILES: *C1CC(C)(N2CCCC2)C1]

In some embodiments, V is N; Y is CH; G is CH₂ and G is connected with the nitrogen of Ring A; Ring A is

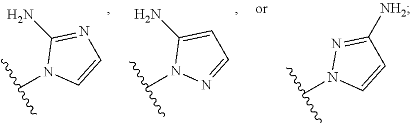 [SMILES: *n1ccnc1N ; *n1nccc1N ; *n1ccc(N)n1]

or and R¹ is ethyl, isopropyl, —CH₂—CHF₂,

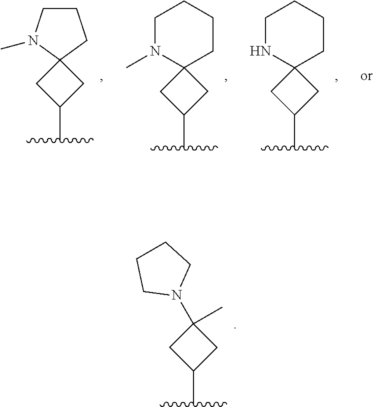

In some embodiments, V is N, Y is CH, G is CH₂ and G is connected with the nitrogen of Ring A; Ring A is

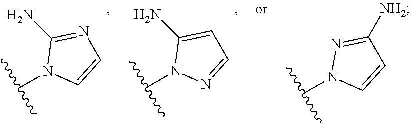 [SMILES: *n1ccnc1N ; *n1nccc1N ; *n1ccc(N)n1]

and R¹ is ethyl, isopropyl, —CH₂—CHF₂, or

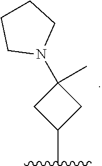 [SMILES: *C1CC(C)(N2CCCC2)C1]

In some embodiments, V is N, Y is CH, G is CH₂ and G is connected with the nitrogen of Ring A; R¹ is ethyl, isopropyl, —CH₂—CHF₂,

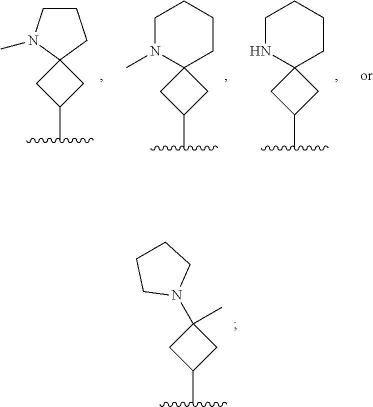

and R² is isopropyl.

In some embodiments, V is N, Y is CH, G is CH₂ and G is connected with the nitrogen of Ring A; R¹ is ethyl, isopropyl, —CH₂—CHF₂,

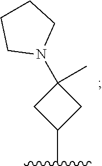 [SMILES: *C1CC(C)(N2CCCC2)C1]

and R² is isopropyl.

In some embodiments, V is N; Y is CH; G is CH₂ and G is connected with the nitrogen of Ring A; Ring A is

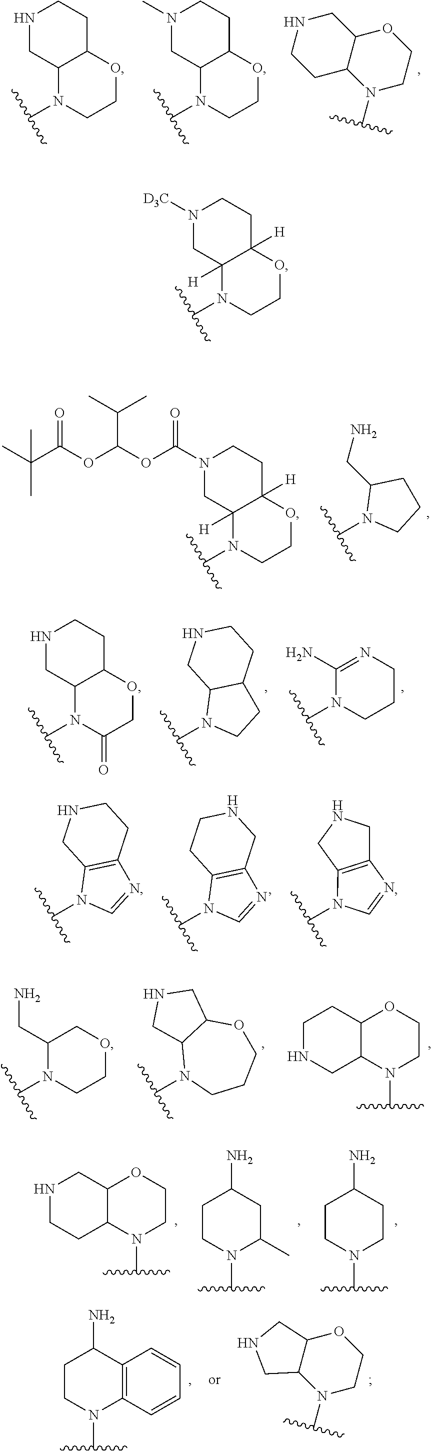

R¹ is ethyl, isopropyl, —CH₂—CHF₂,

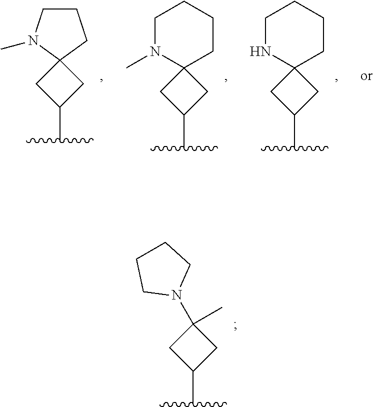

and R² is isopropyl.

In some embodiments, V is N; Y is CH; G is CH₂ and G is connected with the nitrogen of Ring A; Ring A is

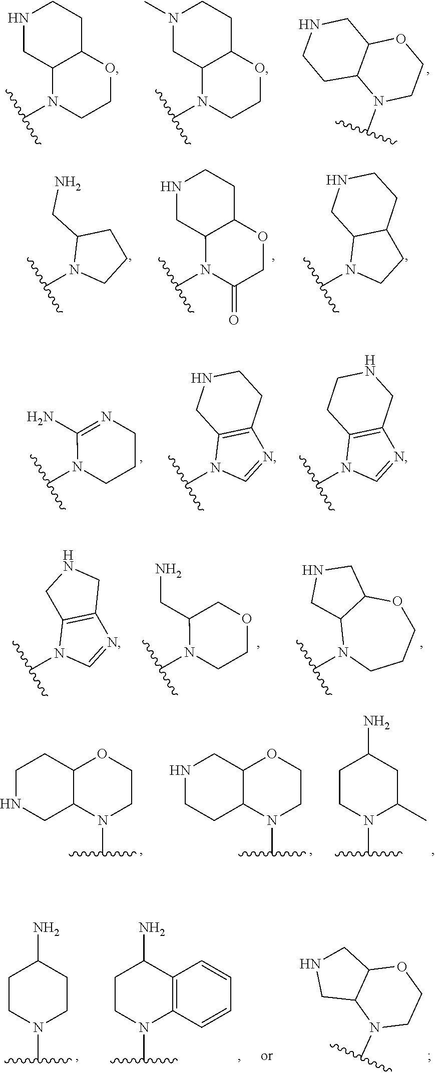

R¹ is ethyl, isopropyl, —CH₂—CHF₂, or

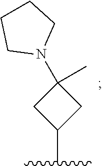 [SMILES: *C1CC(C)(N2CCCC2)C1]

and R² is isopropyl.

In some embodiments, V is N; Y is CH; G is CH₂ and G is connected with the nitrogen of Ring A; Ring A is

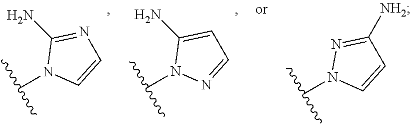 [SMILES: *n1ccnc1N ; *n1nccc1N ; *n1ccc(N)n1]

R¹ is ethyl, isopropyl, —CH₂—CHF₂,

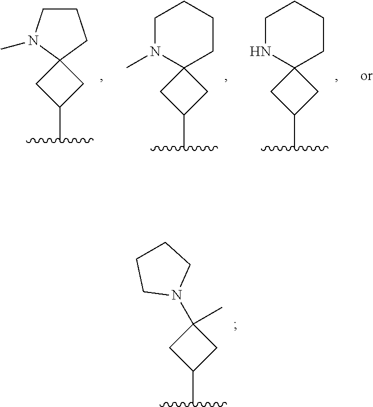

In some embodiments, V is N; Y is CH; G is CH₂ and G is connected with the nitrogen of Ring A; Ring A is

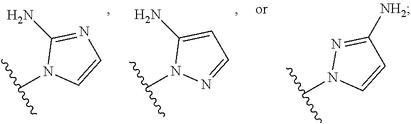 [SMILES: *n1ccnc1N ; *n1nccc1N ; *n1ccc(N)n1]

R¹ is ethyl, isopropyl, —CH₂—CHF₂, or

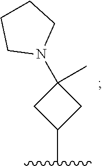 [SMILES: *C1CC(C)(N2CCCC2)C1]

and R² is isopropyl.

In some embodiments, V is N; Y is CH; G is CH₂ and G is connected with the nitrogen of Ring A, and R¹ and R² form

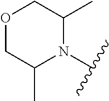 [SMILES: *N1C(C)COCC1C]

group with the nitrogen to which they are connected.

In some embodiments, V is N; Y is CH; G is CH₂ and G is connected with the nitrogen of Ring A; Ring A is

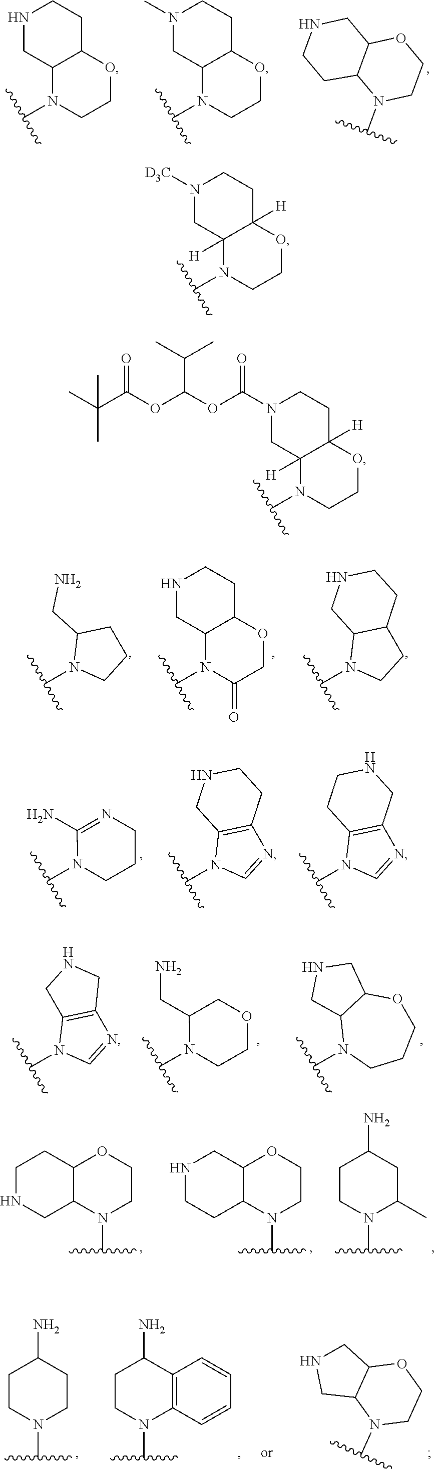

and R¹ and R² form

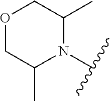 [SMILES: *N1C(C)COCC1C]

group with the nitrogen to which they are connected.

In some embodiments, V is N; Y is CH; G is CH₂ and G is connected with the nitrogen of Ring A; Ring A is

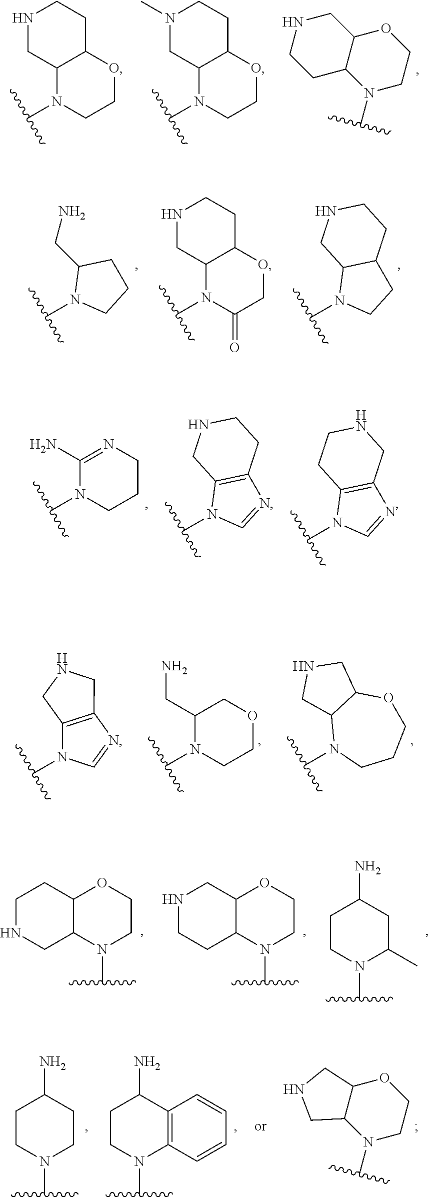

and R¹ and R² form

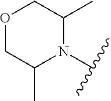 [SMILES: *N1C(C)COCC1C]

group with the nitrogen to which they are connected.

In some embodiments, V is N; Y is CH; G is CH₂ and G is connected with the nitrogen of Ring A; Ring A is

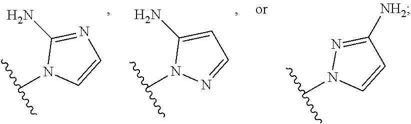 [SMILES: *n1ccnc1N ; *n1nccc1N ; *n1ccc(N)n1]

and R¹ and R² form

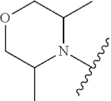 [SMILES: *N1C(C)COCC1C]

group with the nitrogen to which they are connected.

In some embodiments, V is N; Y is CH; G is CH₂ and G is connected with the nitrogen of and the spiro moiety represented by the below formula:

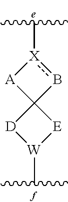

is

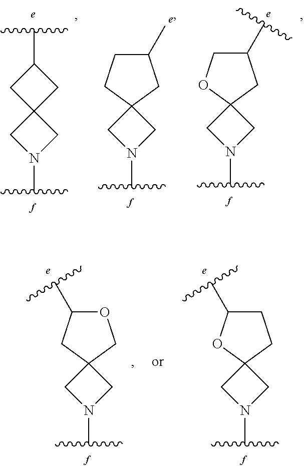

wherein e and f indicate points of attachment to the remainder of the molecule.

In some embodiments, V is N; Y is CH; G is CH₂ and G is connected with the nitrogen of Ring A; Ring A is

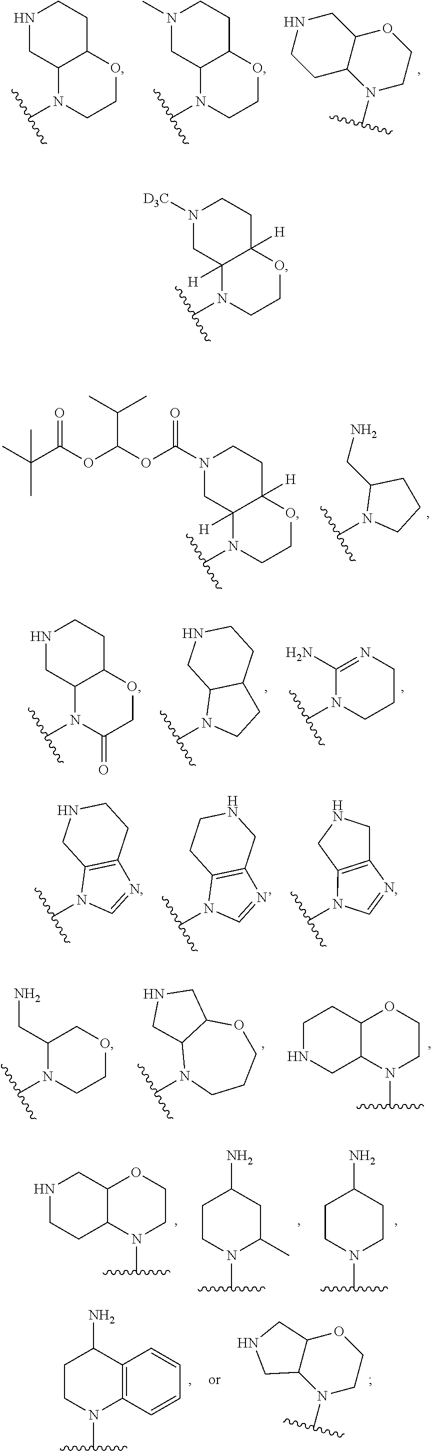

and the spiro moiety represented by the below formula:

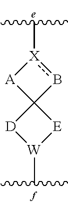

is

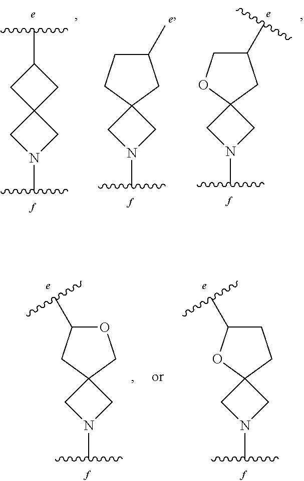

wherein e and f indicate points of attachment to the remainder of the molecule.

In some embodiments, V is N; Y is CH; G is CH₂ and G is connected with the nitrogen of Ring A; Ring A is

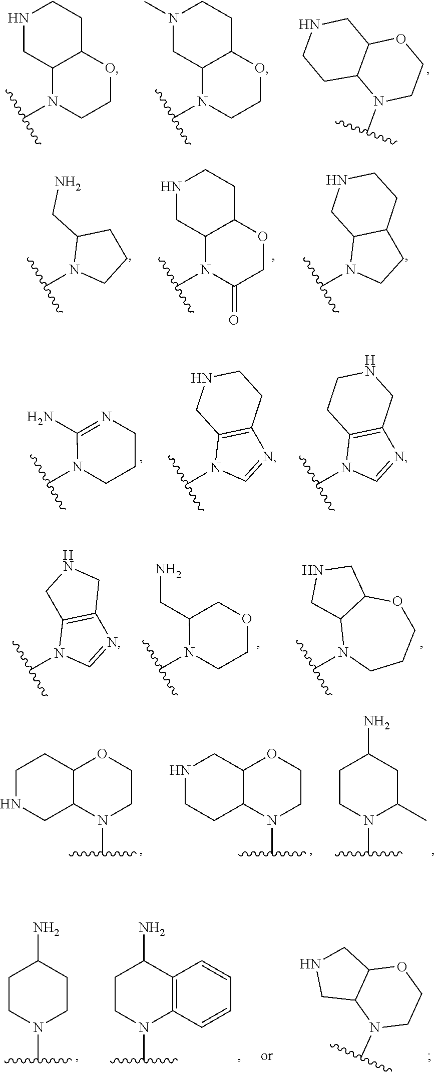

and the spiro moiety represented by the below formula:

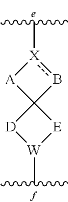

is

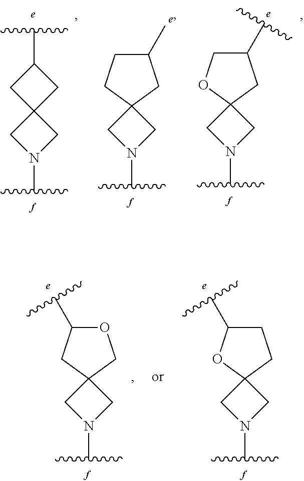

wherein e and f indicate points of attachment to the remainder of the molecule.

In some embodiments, V is N; Y is CH; G is CH₂ and G is connected with the nitrogen of Ring A; Ring A is

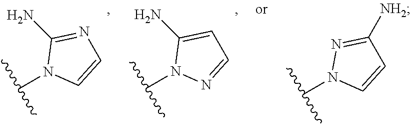 [SMILES: *n1ccnc1N ; *n1nccc1N ; *n1ccc(N)n1]

and the spiro moiety represented by the below formula:

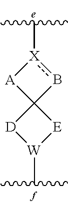

is

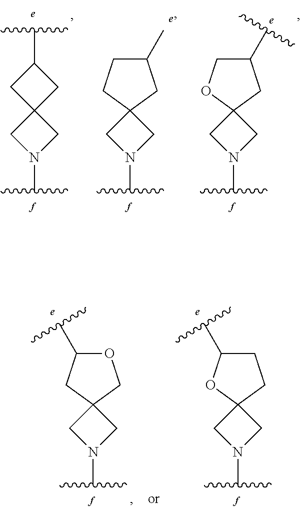

or wherein e and f indicate points of attachment to the remainder of the molecule.

In some embodiments, V is N, Y is CH, G is CH₂ and G is connected with the nitrogen of Ring A, R² is isopropyl; and the spiro moiety represented by the below formula:

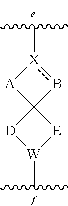

is

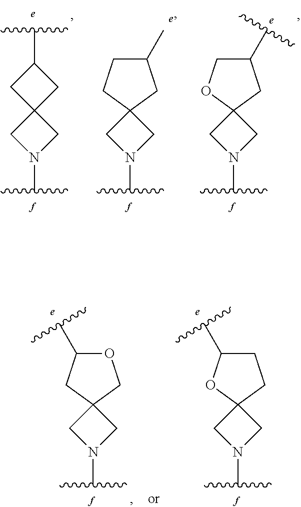

or wherein e and f indicate points of attachment to the remainder of the molecule.

In some embodiments, V is N; Y is CH; G is CH₂ and G is connected with the nitrogen of Ring A; Ring A is

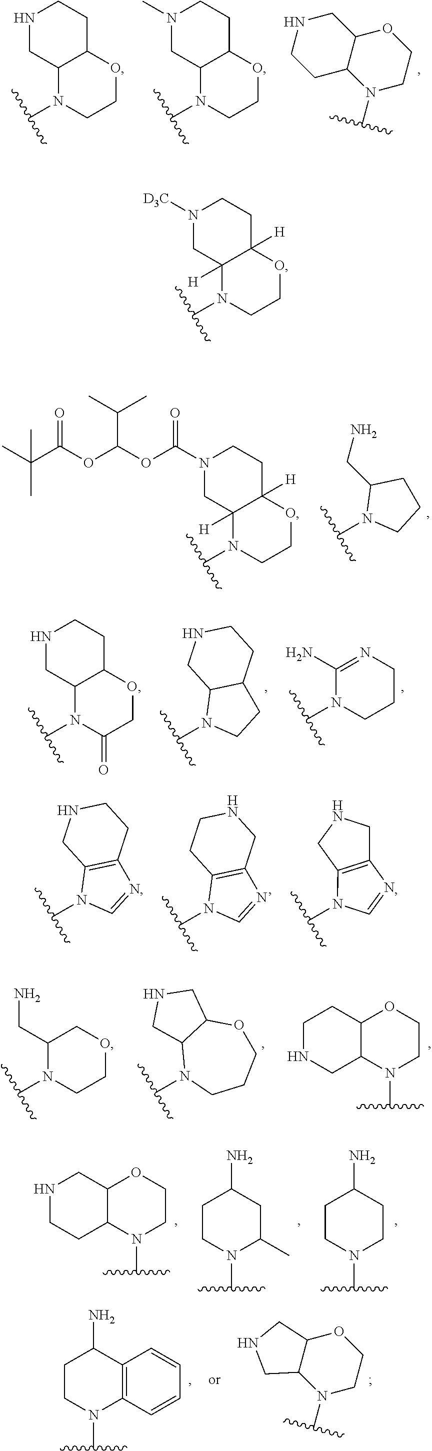

R² is isopropyl; and the spiro moiety represented by the below formula:

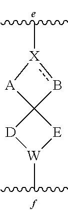

is

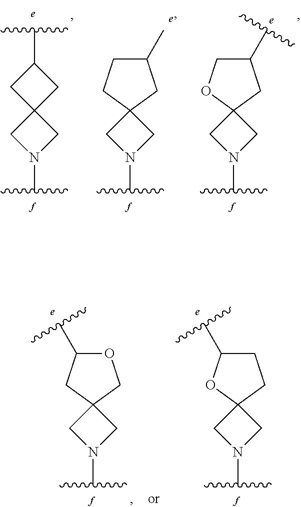

wherein e and f indicate points of attachment to the remainder of the molecule.

In some embodiments, V is N; Y is CH; G is CH₂ and G is connected with the nitrogen of Ring A; Ring A is

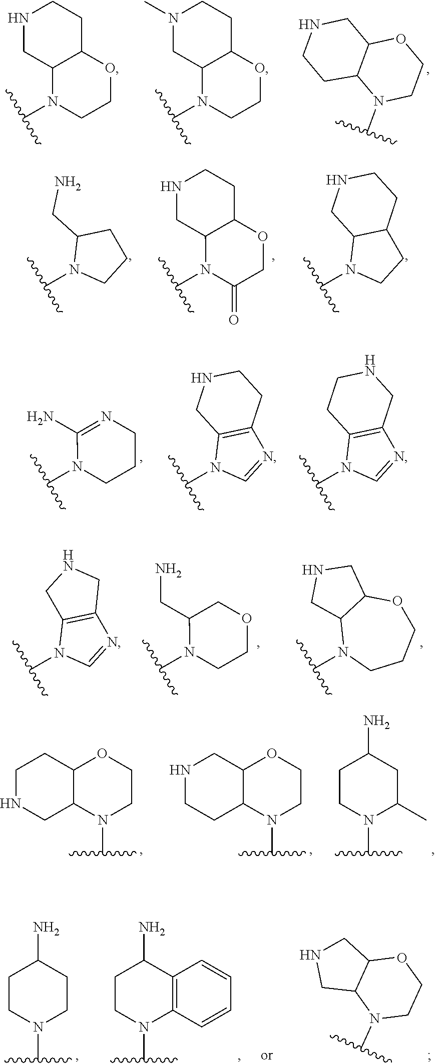

R² is isopropyl; and the spiro moiety represented by the below formula:

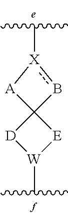

is

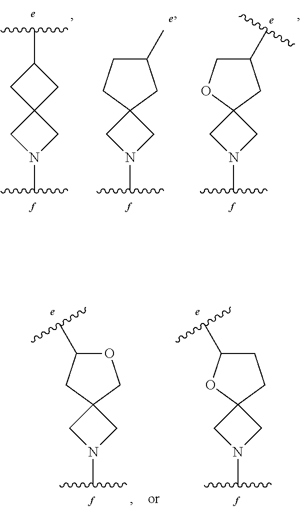

wherein e and f indicate points of attachment to the remainder of the molecule.

In some embodiments, V is N; Y is CH; G is CH₂ and G is connected with the nitrogen of Ring A; Ring A is

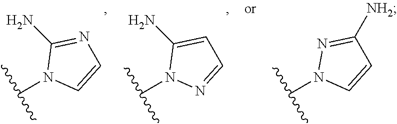 [SMILES: *n1ccnc1N ; *n1nccc1N ; *n1ccc(N)n1]

R² is isopropyl; and the spiro moiety represented by the below formula:

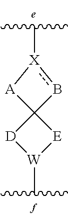

is

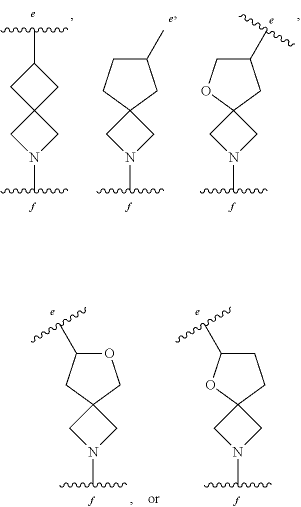

wherein e and f indicate points of attachment to the remainder of the molecule.

In some embodiments, V is N; Y is CH; G is CH₂ and G is connected with the nitrogen of Ring A; R¹ is ethyl, isopropyl, —CH₂—CF₂,

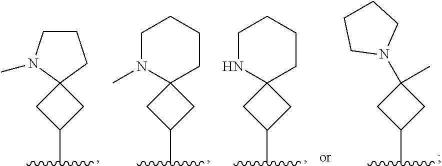

and the spiro moiety represented by the below formula:

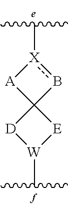

is

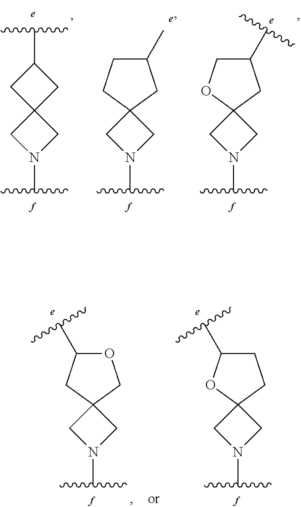

wherein e and f indicate points of attachment to the remainder of the molecule.

In some embodiments, V is N; Y is CH; G is CH₂ and G is connected with the nitrogen of Ring A; R¹ is ethyl, isopropyl, —CH₂—CHF₂,

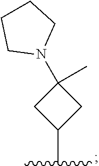 [SMILES: *C1CC(C)(N2CCCC2)C1]

and the spiro moiety represented by the below formula:

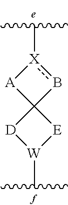

is

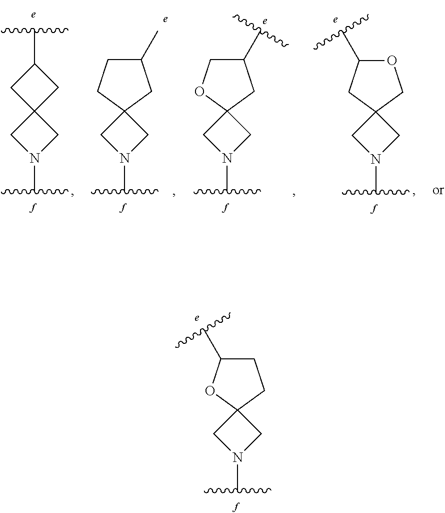

wherein e and f indicate points of attachment to the remainder of the molecule.

In some embodiments, V is N; Y is CH; G is CH₂ and G is connected with the nitrogen of Ring A; Ring A is

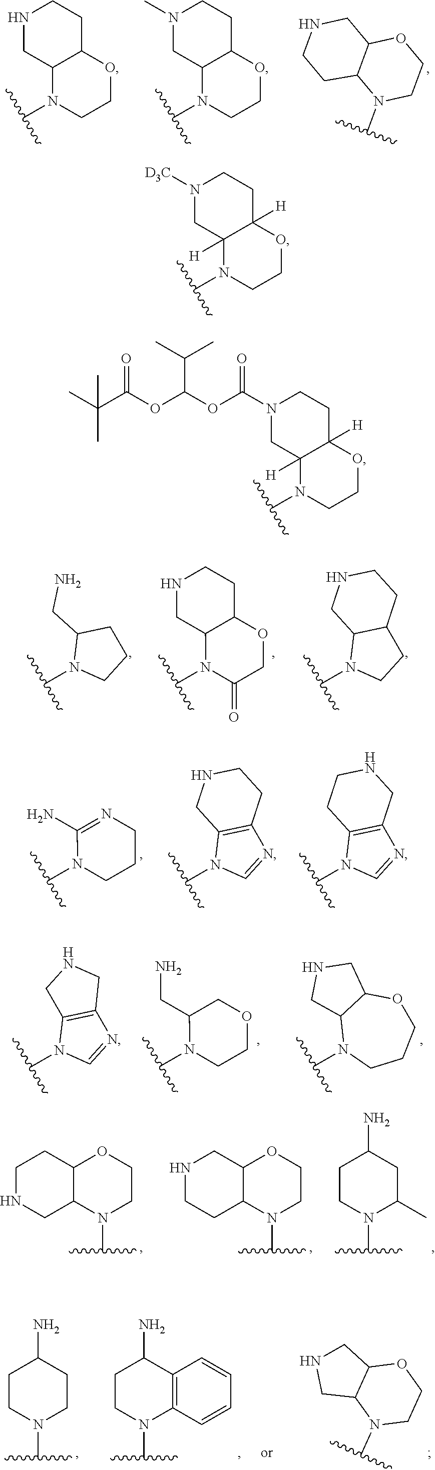

R¹ is ethyl, isopropyl, —CH₂—CHF₂,

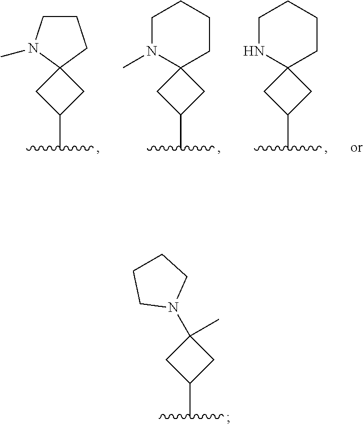

and the spiro moiety represented by the below formula:

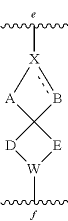

is

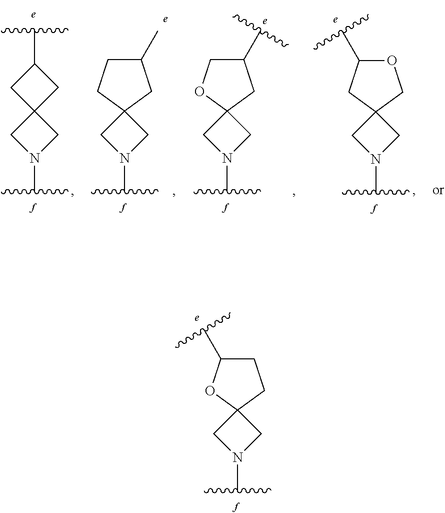

wherein e and f indicate points of attachment to the remainder of the molecule.

In some embodiments, V is N; Y is CH; G is CH₂ and G is connected with the nitrogen of Ring A; Ring A is

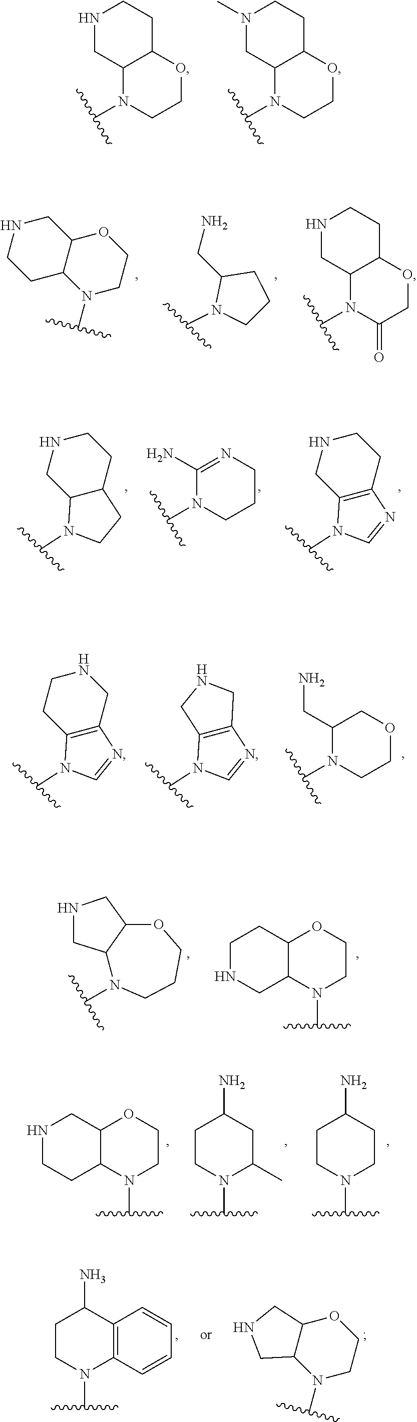

R¹ is ethyl, isopropyl, —CH₂—CHF₂, or

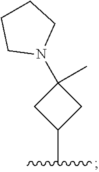 [SMILES: *C1CC(C)(N2CCCC2)C1]

and the spiro moiety represented by the below formula:

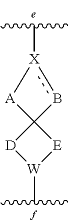

is

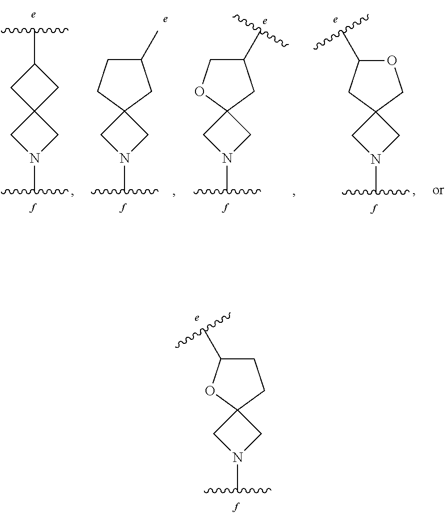

wherein e and f indicate points of attachment to the remainder of the molecule.

In some embodiments, V is N; Y is CH; G is CH₂ and G is connected with the nitrogen of Ring A; Ring A is

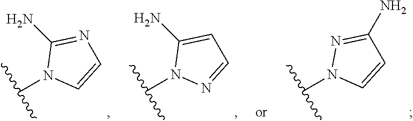 [SMILES: *n1ccnc1N ; *n1nccc1N ; *n1ccc(N)n1]

R¹ is ethyl, isopropyl, —CH₂—CHF₂,

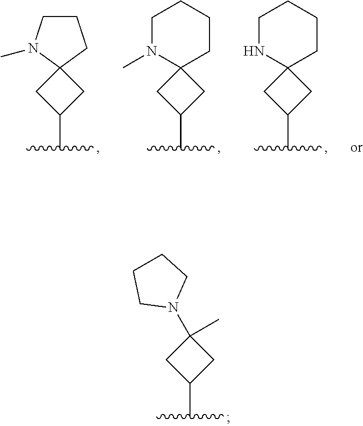

and the spiro moiety represented by the below formula:

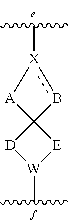

is

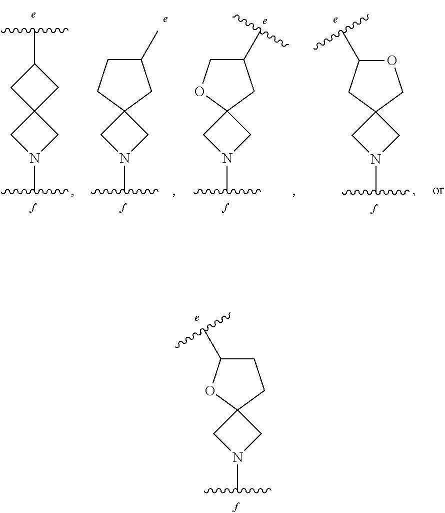

wherein e and f indicate points of attachment to the remainder of the molecule.

In some embodiments, V is N; Y is CH; G is CH₂ and G is connected with the nitrogen of Ring A; Ring A is

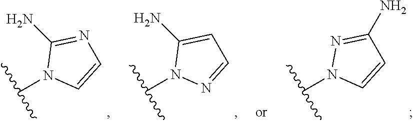 [SMILES: *n1ccnc1N ; *n1nccc1N ; *n1ccc(N)n1]

R¹ is ethyl, isopropyl, —CH₂—CHF₂, or

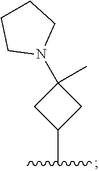 [SMILES: *C1CC(C)(N2CCCC2)C1]

and the spiro moiety represented by the below formula:

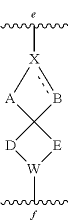

is

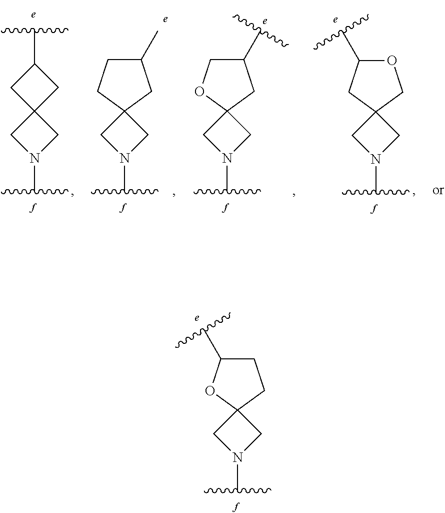

wherein e and f indicate points of attachment to the remainder of the molecule.

In some embodiments, V is N, Y is CH, G is CH₂ and G is connected with the nitrogen of Ring A; R¹ is ethyl, isopropyl, —CH₂—CHF₂,

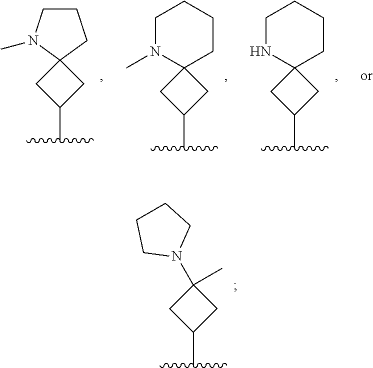

R² is isopropyl; and the spiro moiety represented by the below formula:

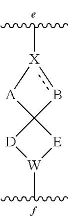

is

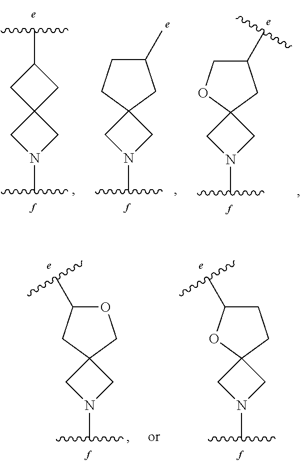

or wherein e and f indicate points of attachment to the remainder of the molecule.

In some embodiments, V is N, Y is CH, G is CH₂ and G is connected with the nitrogen of Ring A; R¹ is ethyl, isopropyl, —CH₂—CHF₂, or

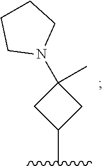 [SMILES: *C1CC(C)(N2CCCC2)C1]

R² is isopropyl; and the spiro moiety represented by the below formula:

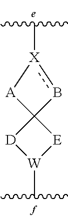

is

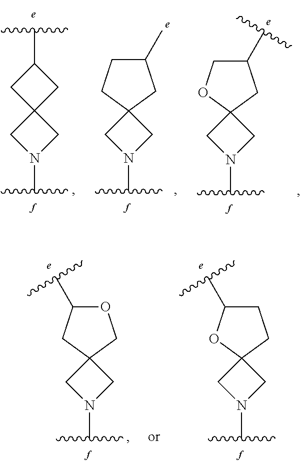

wherein e and f indicate points of attachment to the remainder of the molecule.

In some embodiments, V is N; Y is CH; G is CH₂ and G is connected with the nitrogen of Ring A; Ring A is

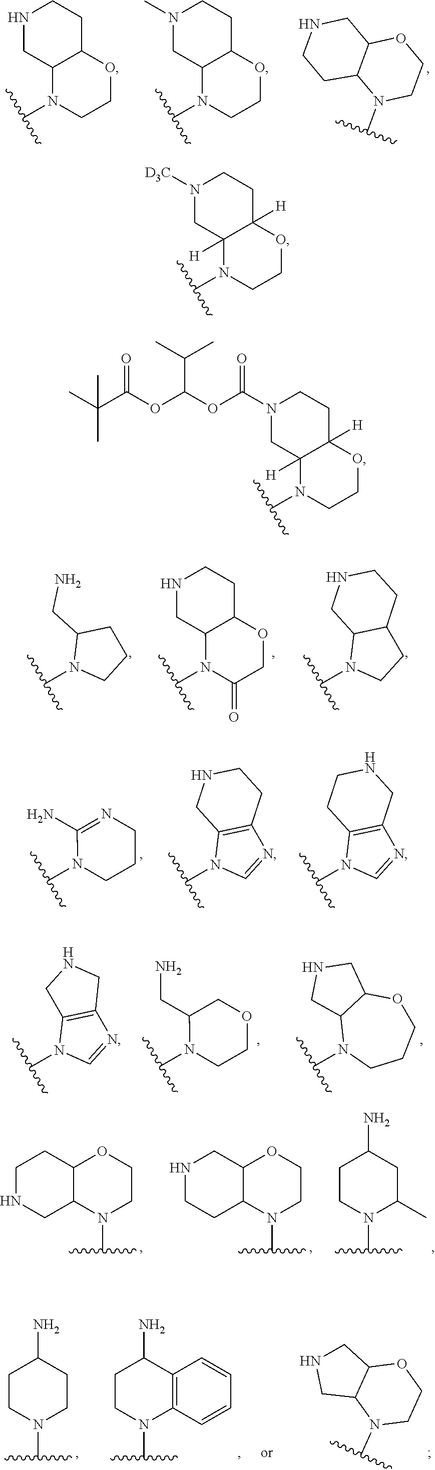

R¹ is ethyl, isopropyl, —CH₂—CHF₂,

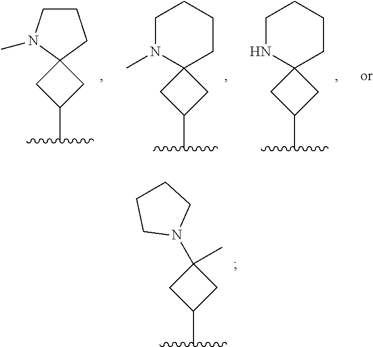

R² is isopropyl; and the spiro moiety represented by the below formula:

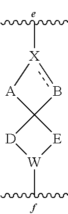

is

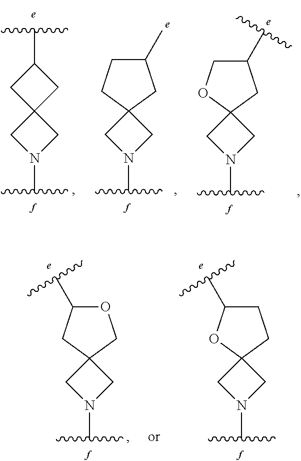

wherein e and f indicate points of attachment to the remainder of the molecule.

In some embodiments, V is N; Y is CH; G is CH₂ and G is connected with the nitrogen of Ring A; Ring A is

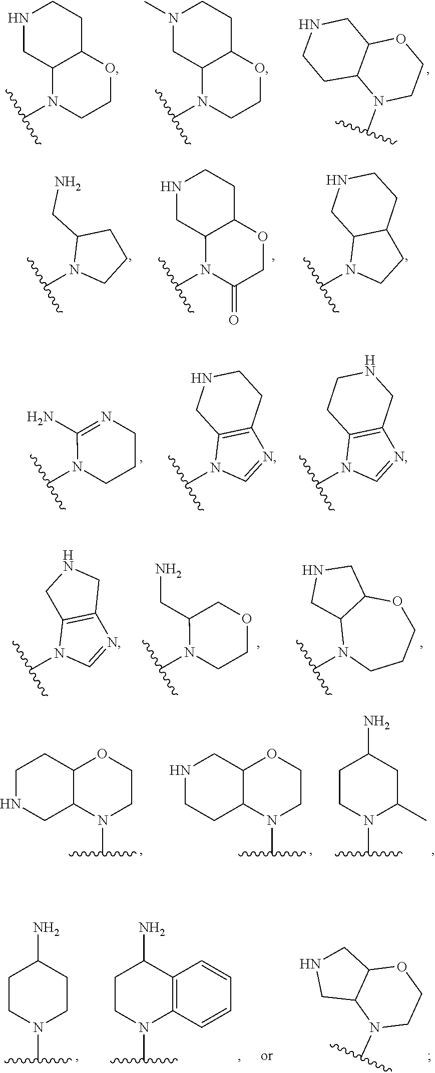

R¹ is ethyl, isopropyl, —CH₂—CHF₂, or

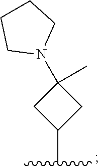 [SMILES: *C1CC(C)(N2CCCC2)C1]

R² is isopropyl; and the spiro moiety represented by the below formula:

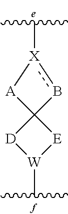

is

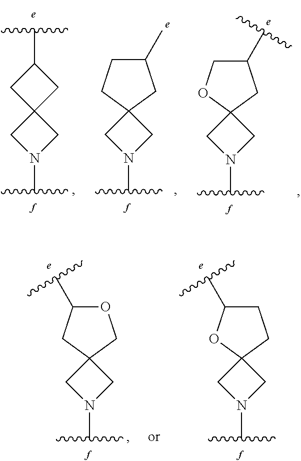

wherein e and f indicate points of attachment to the remainder of the molecule.

In some embodiments, V is N; Y is CH; G is CH₂ and G is connected with the nitrogen of Ring A; Ring A is

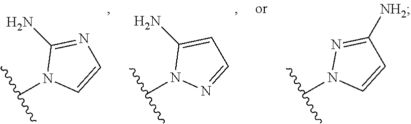 [SMILES: *n1ccnc1N ; *n1nccc1N ; *n1ccc(N)n1]

R¹ is ethyl, isopropyl, —CH₂—CHF₂,

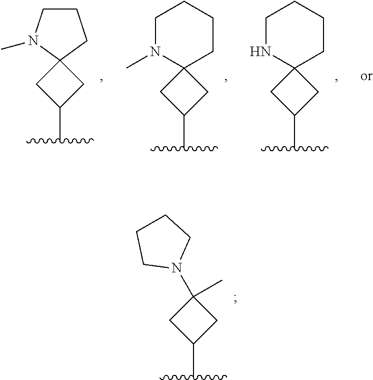

R² is isopropyl; and the spiro moiety represented by the below formula:

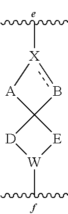

is

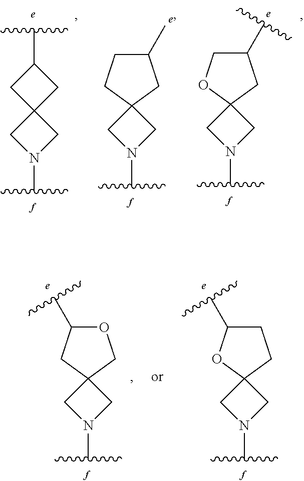

wherein e and f indicate points of attachment to the remainder of the molecule.

In some embodiments, V is N; Y is CH; G is CH₂ and G is connected with the nitrogen of Ring A; Ring A is

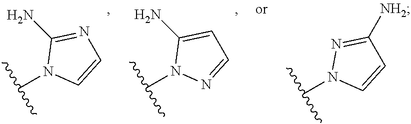 [SMILES: *n1ccnc1N ; *n1nccc1N ; *n1ccc(N)n1]

R¹ is ethyl, isopropyl, —CH₂—CHF₂, or

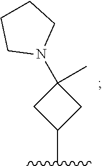 [SMILES: *C1CC(C)(N2CCCC2)C1]

R² is isopropyl; and the spiro moiety represented by the below formula:

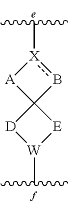

is

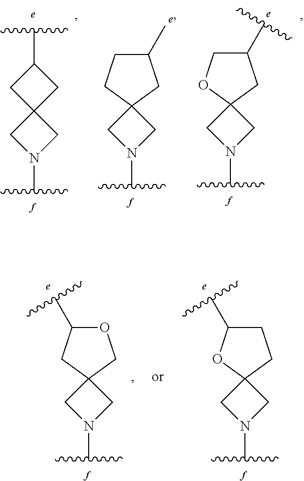

wherein e and f indicate points of attachment to the remainder of the molecule.

In some embodiments, V is N; Y is CH; G is CH₂ and G is connected with the nitrogen of Ring A; R¹ and R² form

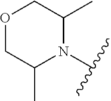 [SMILES: *N1C(C)COCC1C]

group with the nitrogen to which they are connected; and the spiro moiety represented by the below formula:

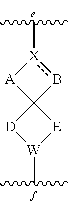

is

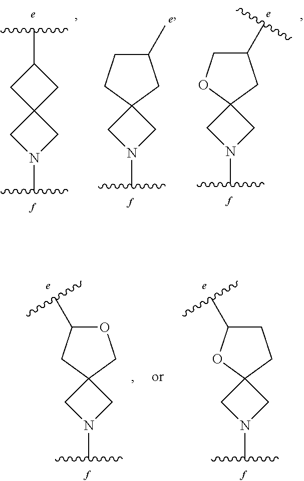

wherein e and f indicate points of attachment to the remainder of the molecule.

In some embodiments V is N; Y is CH, G is CH₂ and G is connected with the nitrogen of Ring A; Ring A is

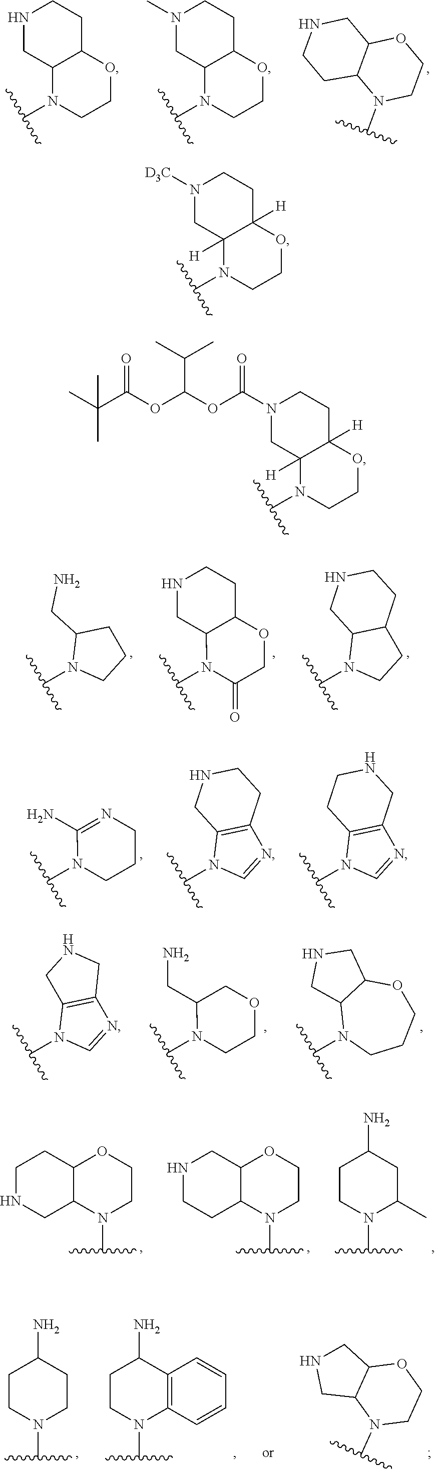

R¹ and R² form

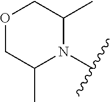 [SMILES: *N1C(C)COCC1C]

group with the nitrogen to which they are connected; and the spiro moiety represented by the below formula:

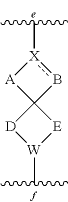

is

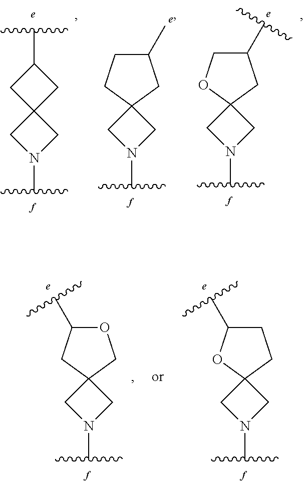

wherein e and f indicate points of attachment to the remainder of the molecule.

In some embodiments, V is N; Y is CH; G is CH₂ and G is connected with the nitrogen of Ring A; Ring A is

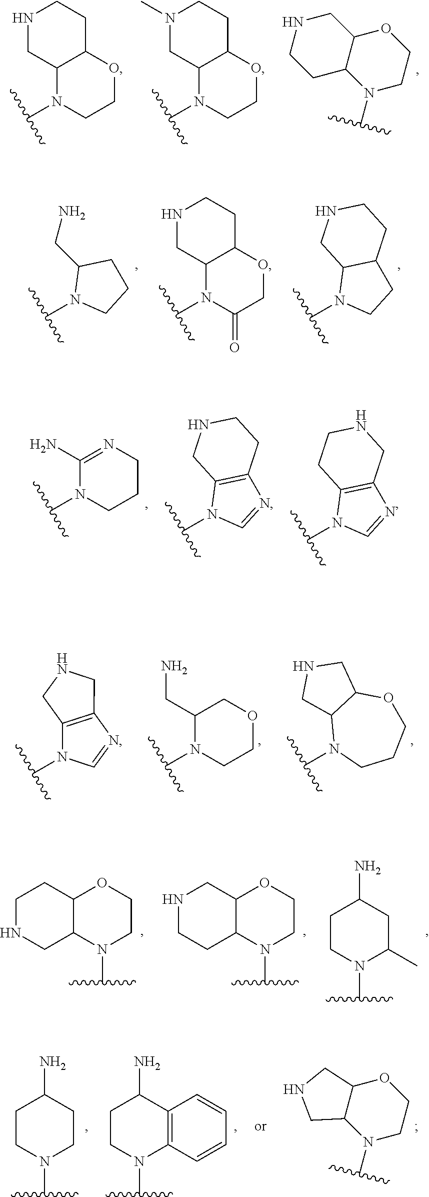

R¹ and R² form

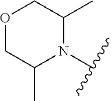 [SMILES: *N1C(C)COCC1C]

group with the nitrogen to which they are connected; and the spiro moiety represented by the below formula:

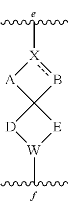

is

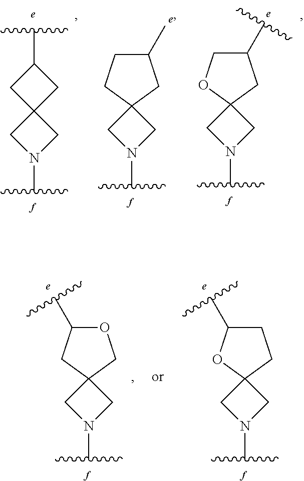

wherein e and f indicate points of attachment to the remainder of the molecule.

In some embodiments, V is N; Y is CH; G is CH₂ and G is connected with the nitrogen of Ring A; Ring A is

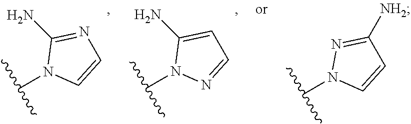 [SMILES: *n1ccnc1N ; *n1nccc1N ; *n1ccc(N)n1]

R¹ and R² form

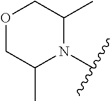 [SMILES: *N1C(C)COCC1C]

group with the nitrogen to which they are connected; and the spiro moiety represented by the below formula:

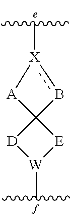

is

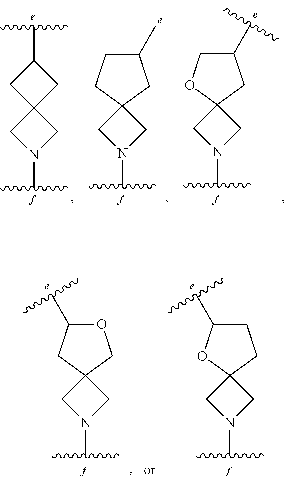

wherein e and f indicate points of attachment to the remainder of the molecule.

In some embodiments, the compound is of Formula I, II, II-a, II-b, II-c, II-d, or II-e combined with any of the embodiments described herein.

Any of the groups described above for any variable can be combined with any of the other groups described above, where applicable, for any of the Formulae described herein.

Representative compounds of the present disclosure are shown in the table below.

TABLE 1
Representative Compounds of the Present Disclosure

| Compound | | |
|---|---|---|
| No. | Structure | IUPAC Name |
| 1 | 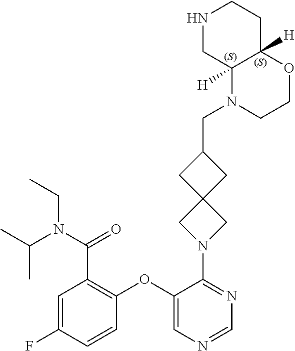 | N-Ethyl-5-fluoro-N-isopropyl-2-((4- (6-(((4aS,8aS)-octahydro-4H- pyrido[4,3-b][1,4]oxazin-4- yl)methyl)-2-azaspiro[3.3]heptan-2- yl)pyrimidin-5-yl)oxy)benzamide |
| 2 | 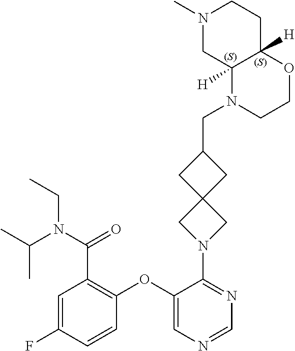 | N-Ethyl-5-fluoro-N-isopropyl-2-((4- (6-(((4aS,8aS)-6-methyloctahydro-4H- pyrido[4,3-b][1,4]oxazin-4- yl)methyl)-2-azaspiro[3.3]heptan-2- yl)pyrimidin-5-yl)oxy)benzamide |
| 3 | 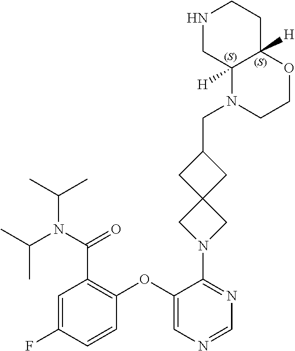 | 5-Fluoro-N,N-diisopropyl-2-((4-(6- (((4aS,8aS)-octahydro-4H-pyrido[4,3- b][1,4]oxazin-4-yl)methyl)-2- azaspiro[3.3]heptan-2-yl)pyrimidin-5- yl)oxy)benzamide |
| 4 | 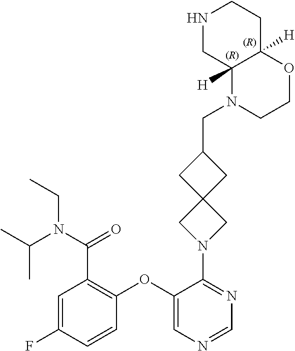 | N-Ethyl-5-fluoro-N-isopropyl-2-((4- (6-(((4aR,8aR)-octahydro-4H- pyrido[4,3-b][1,4]oxazin-4- yl)methyl)-2-azaspiro[3.3]heptan-2- yl)pyrimidin-5-yl)oxy)benzamide |
| 5 | 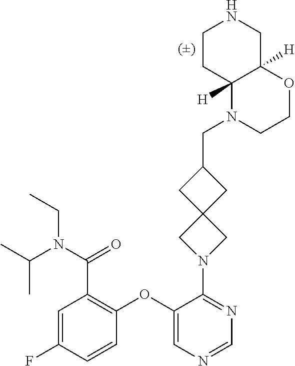 [SMILES: CCN(C(=O)c1cc(F)ccc1Oc1cncnc1N1CC2(CC(CN3CCO[C@@H]4CNCC[C@H]43)C2)C1)C(C)C] | (±)-N-Ethyl-5-fluoro-N-isopropyl-2- ((4-(6-(((trans)-octahydro-1H- pyrido[3,4-b][1,4]oxazin-1- yl)methyl)-2-azaspiro[3.3]heptan-2- yl)pyrimidin-5-yl)oxy)benzamide |
| 6 | 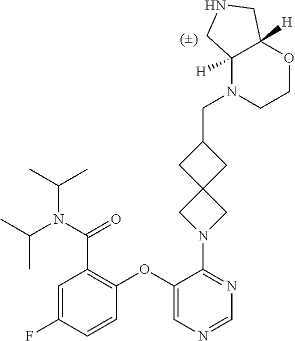 [SMILES: CC(C)N(C(=O)c1cc(F)ccc1Oc1cncnc1N1CC2(CC(CN3CCO[C@H]4CNC[C@@H]43)C2)C1)C(C)C] | (±)-5-Fluoro-2-((4-(6-(((trans)- hexahydropyrrolo[3,4-b][1,4]oxazin- 4(4aH)-yl)methyl)-2- azaspiro[3.3]heptan-2-yl)pyrimidin-5- yl)oxy)-N,N-diisopropylbenzamide |
| 7-a | 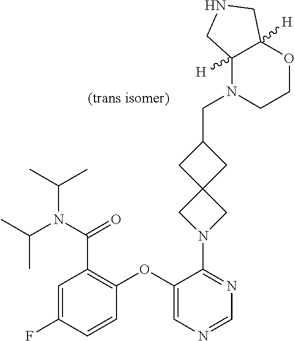 [SMILES: CC(C)N(C(=O)c1cc(F)ccc1Oc1cncnc1N1CC2(CC(CN3CCOC4CNCC43)C2)C1)C(C)C] | 5-Fluoro-2-((4-(6-(((trans)- hexahydropyrrolo[3,4-b][1,4]oxazin- 4(4aH)-yl)methyl)-2- azaspiro[3.3]heptan-2-yl)pyrimidin-5- yl)oxy)-N,N-diisopropylbenzamide |
| 7-b | 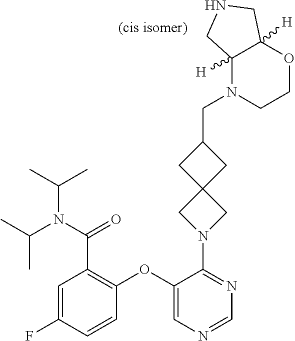 [SMILES: CC(C)N(C(=O)c1cc(F)ccc1Oc1cncnc1N1CC2(CC(CN3CCOC4CNCC43)C2)C1)C(C)C] | 5-Fluoro-2-((4-(6-(((cis)- hexahydropyrrolo[3,4-b][1,4]oxazin- 4(4aH)-yl)methyl)-2- azaspiro[3.3]heptan-2-yl)pyrimidin-5- yl)oxy)-N,N-diisopropylbenzamide |
| 7 | 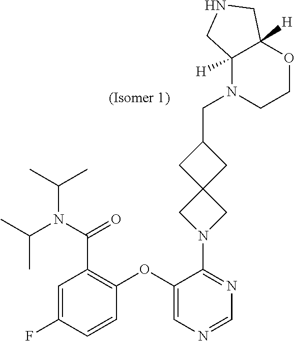 [SMILES: CC(C)N(C(=O)c1cc(F)ccc1Oc1cncnc1N1CC2(CC(CN3CCO[C@H]4CNC[C@@H]43)C2)C1)C(C)C] | (E1)-5-Fluoro-2-((4-(6-(((trans)- hexahydropyrrolo[3,4-b][1,4]oxazin- 4(4aH)-yl)methyl)-2- azaspiro[3.3]heptan-2-yl)pyrimidin-5- yl)oxy)-N,N-diisopropylbenzamide |
| 8 | 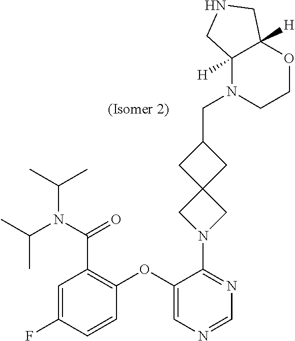 [SMILES: CC(C)N(C(=O)c1cc(F)ccc1Oc1cncnc1N1CC2(CC(CN3CCO[C@H]4CNC[C@@H]43)C2)C1)C(C)C] | (E2)-5-Fluoro-2-((4-(6-(((trans)- hexahydropyrrolo[3,4-b][1,4]oxazin- 4(4aH)-yl)methyl)-2- azaspiro[3.3]heptan-2-yl)pyrimidin-5- yl)oxy)-N,N-diisopropylbenzamide |
| 9-a | 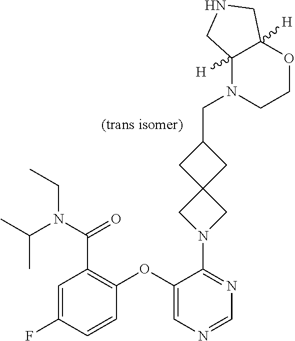 [SMILES: CCN(C(=O)c1cc(F)ccc1Oc1cncnc1N1CC2(CC(CN3CCOC4CNCC43)C2)C1)C(C)C] | N-Ethyl-5-fluoro-2-((4-(6-(((trans)- hexahydropyrrolo[3,4-b][1,4]oxazin- 4(4aH)-yl)methyl)-2- azaspiro[3.3]heptan-2-yl)pyrimidin-5- yl)oxy)-N-isopropylbenzamide |
| 9-b | 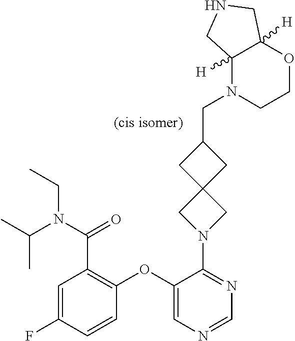 [SMILES: CCN(C(=O)c1cc(F)ccc1Oc1cncnc1N1CC2(CC(CN3CCOC4CNCC43)C2)C1)C(C)C] | N-Ethyl-5-fluoro-2-((4-(6-(((cis)- hexahydropyrrolo[3,4-b][1,4]oxazin- 4(4aH)-yl)methyl)-2- azaspiro[3.3]heptan-2-yl)pyrimidin-5- yl)oxy)-N-isopropylbenzamide |
| 9 | 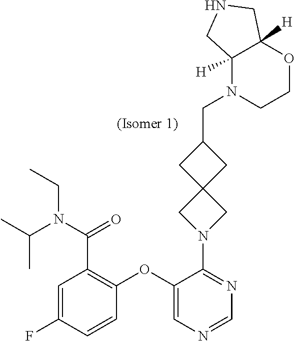 [SMILES: CCN(C(=O)c1cc(F)ccc1Oc1cncnc1N1CC2(CC(CN3CCO[C@H]4CNC[C@@H]43)C2)C1)C(C)C] | (E1)-N-Ethyl-5-fluoro-2-((4-(6- (((trans)-hexahydropyrrolo[3,4- b][1,4]oxazin-4(4aH)-yl)methyl)-2- azaspiro[3.3]heptan-2-yl)pyrimidin-5- yl)oxy)-N-isopropylbenzamide |
| 10 | 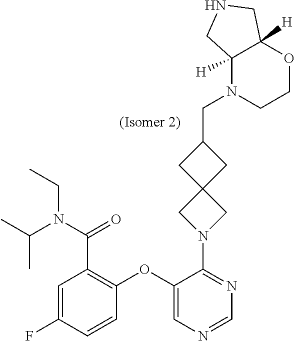 [SMILES: CCN(C(=O)c1cc(F)ccc1Oc1cncnc1N1CC2(CC(CN3CCO[C@H]4CNC[C@@H]43)C2)C1)C(C)C] | (E2)-N-Ethyl-5-fluoro-2-((4-(6- (((trans)-hexahydropyrrolo[3,4- b][1,4]oxazin-4(4aH)-yl)methyl)-2- azaspiro[3.3]heptan-2-yl)pyrimidin-5- yl)oxy)-N-isopropylbenzamide |
| 11 | 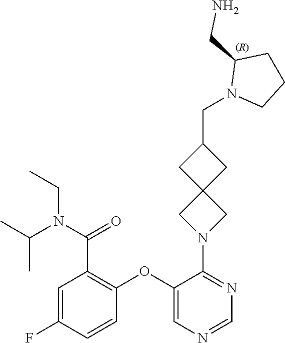 | (R)-2-((4-(6-((2- (Aminomethyl)pyrrolidin-1- yl)methyl)-2-azaspiro[3.3]heptan-2- yl)pyrimidin-5-yl)oxy)-N-ethyl-5- fluoro-N-isopropylbenzamide |
| 12 | 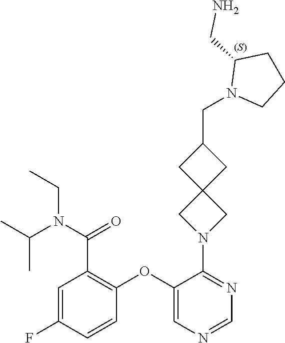 | (S)-2-((4-(6-((2- (Aminomethyl)pyrrolidin-1- yl)methyl)-2-azaspiro[3.3]heptan-2- yl)pyrimidin-5-yl)oxy)-N-ethyl-5- fluoro-N-isopropylbenzamide |
| 13 | 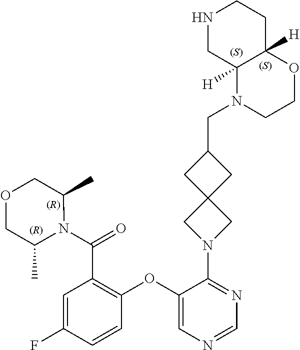 | ((3R,5R)-3,5-Dimethylmorpholino)(5- fluoro-2-((4-(6-(((4aS,8aS)-octahydro- 4H-pyrido[4,3-b][1,4]oxazin-4- yl)methyl)-2-azaspiro[3.3]heptan-2- yl)pyrimidin-5- yl)oxy)phenyl)methanone |
| 14 | 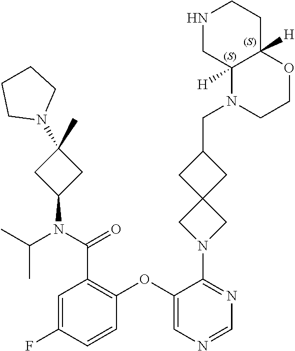 | 5-Fluoro-N-isopropyl-N-((1r,3r)-3- methyl-3-(pyrrolidin-1-yl)cyclobutyl)- 2-((4-(6-(((4aS,8aS)-octahydro-4H- pyrido[4,3-b][1,4]oxazin-4- yl)methyl)-2-azaspiro[3.3]heptan-2- yl)pyrimidin-5-yl)oxy)benzamide |
| 15 | 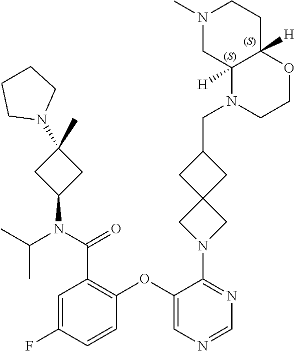 | 5-Fluoro-N-isopropyl-N-((1r,3r)-3- methyl-3-(pyrrolidin-1-yl)cyclobutyl)- 2-((4-(6-(((4aS,8aS)-6- methyloctahydro-4H-pyrido[4,3- b][1,4]oxazin-4-yl)methyl)-2- azaspiro[3.3]heptan-2-yl)pyrimidin-5- yl)oxy)benzamide |
| 16 | 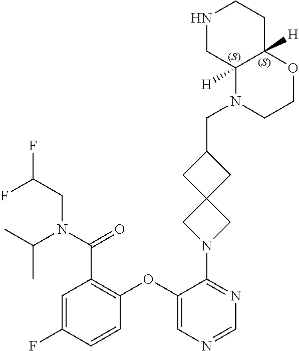 | N-(2,2-Difluoroethyl)-5-fluoro-N- isopropyl-2-((4-(6-(((4aS,8aS)- octahydro-4H-pyrido[4,3- b][1,4]oxazin-4-yl)methyl)-2- azaspiro[3.3]heptan-2-yl)pyrimidin-5- yl)oxy)benzamide |
| 17 | 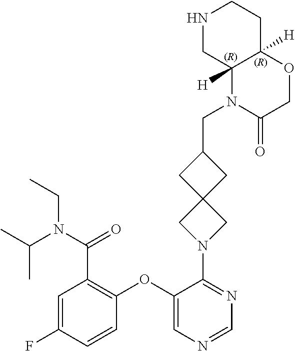 | N-Ethyl-5-fluoro-N-isopropyl-2-((4- (6-(((4aR,8aR)-3-oxooctahydro-4H- pyrido[4,3-b][1,4]oxazin-4- yl)methyl)-2-azaspiro[3.3]heptan-2- yl)pyrimidin-5-yl)oxy)benzamide |
| 18 | 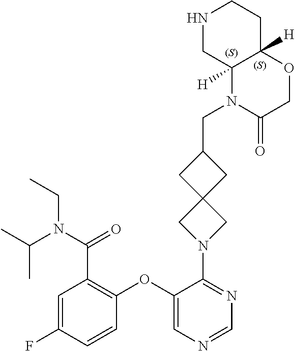 | N-Ethyl-5-fluoro-N-isopropyl-2-((4- (6-(((4aS,8aS)-3-oxooctahydro-4H- pyrido[4,3-b][1,4]oxazin-4- yl)methyl)-2-azaspiro[3.3]heptan-2- yl)pyrimidin-5-yl)oxy)benzamide |
| 19 | 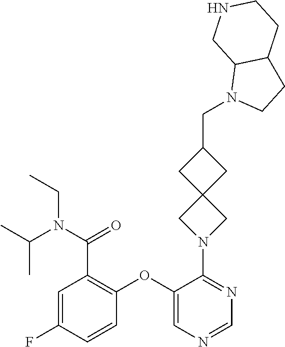 [SMILES: CCN(C(=O)c1cc(F)ccc1Oc1cncnc1N1CC2(CC(CN3CCC4CCNCC43)C2)C1)C(C)C] | N-Ethyl-5-fluoro-N-isopropyl-2-((4- (6-((octahydro-1H-pyrrolo[2,3- c]pyridin-1-yl)methyl)-2- azaspiro[3.3]heptan-2-yl)pyrimidin-5- yl)oxy)benzamide |
| 20 | 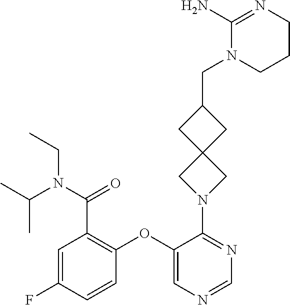 [SMILES: CCN(C(=O)c1cc(F)ccc1Oc1cncnc1N1CC2(CC(CN3CCCN=C3N)C2)C1)C(C)C] | 2-((4-(6-((2-Amino-5,6- dihydropyrimidin-1(4H)-yl)methyl)-2- azaspiro[3.3]heptan-2-yl)pyrimidin-5- yl)oxy)-N-ethyl-5-fluoro-N- isopropylbenzamide |
| 21 | 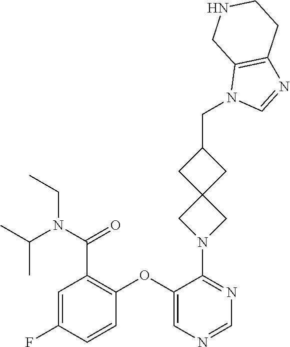 [SMILES: CCN(C(=O)c1cc(F)ccc1Oc1cncnc1N1CC2(CC(Cn3cnc4c3CNCC4)C2)C1)C(C)C] | N-Ethyl-5-fluoro-N-isopropyl-2-((4- (6-((4,5,6,7-tetrahydro-3H- imidazo[4,5-c]pyridin-3-yl)methyl)-2- azaspiro[3.3]heptan-2-yl)pyrimidin-5- yl)oxy)benzamide |
| 22 | 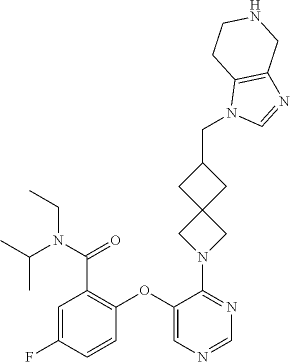 [SMILES: CCN(C(=O)c1cc(F)ccc1Oc1cncnc1N1CC2(CC(Cn3cnc4c3CCNC4)C2)C1)C(C)C] | N-Ethyl-5-fluoro-N-isopropyl-2-((4- (6-((4,5,6,7-tetrahydro-1H- imidazo[4,5-c]pyridin-1-yl)methyl)-2- azaspiro[3.3]heptan-2-yl)pyrimidin-5- yl)oxy)benzamide |
| 23 | 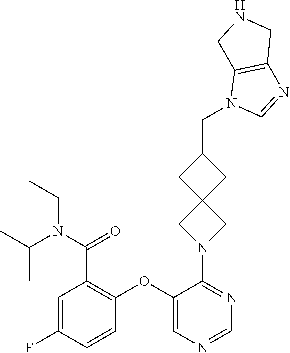 [SMILES: CCN(C(=O)c1cc(F)ccc1Oc1cncnc1N1CC2(CC(Cn3cnc4c3CNC4)C2)C1)C(C)C] | 2-((4-(6-((5,6-Dihydropyrrolo[3,4- d]imidazol-1(4H)-yl)methyl)-2- azaspiro[3.3]heptan-2-yl)pyrimidin-5- yl)oxy)-N-ethyl-5-fluoro-N- isopropylbenzamide |
| 24 | 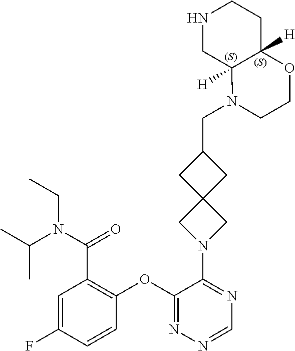 | N-Ethyl-5-fluoro-N-isopropyl-2-((5- (6-(((4aS,8aS)-octahydro-4H- pyrido[4,3-b][1,4]oxazin-4- yl)methyl)-2-azaspiro[3.3]heptan-2- yl)-1,2,4-triazin-6-yl)oxy)benzamide |
| 25 | 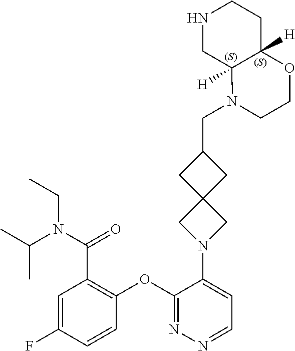 | N-Ethyl-5-fluoro-N-isopropyl-2-((4- (6-(((4aS,8aS)-octahydro-4H- pyrido[4,3-b][1,4]oxazin-4- yl)methyl)-2-azaspiro[3.3]heptan-2- yl)pyridazin-3-yl)oxy)benzamide |
| 26 | 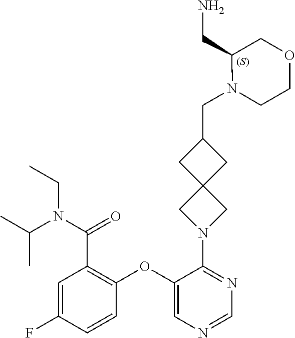 | (S)-2-((4-(6-((3- (Aminomethyl)morpholino)methyl)-2- azaspiro[3.3]heptan-2-yl)pyrimidin-5- yl)oxy)-N-ethyl-5-fluoro-N- isopropylbenzamide |
| 27 | 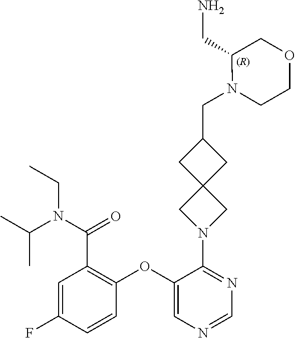 | (R)-2-((4-(6-((3- (Aminomethyl)morpholino)methyl)-2- azaspiro[3.3]heptan-2-yl)pyrimidin-5- yl)oxy)-N-ethyl-5-fluoro-N- isopropylbenzamide |
| 28 | 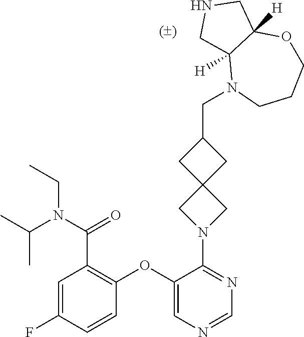 [SMILES: CCN(C(=O)c1cc(F)ccc1Oc1cncnc1N1CC2(CC(CN3CCCO[C@H]4CNC[C@@H]43)C2)C1)C(C)C] | (±)-N-Ethyl-5-fluoro-2-((4-(6- (((trans)-hexahydro-2H-pyrrolo[3,4- b][1,4]oxazepin-5(5aH)-yl)methyl)-2- azaspiro[3.3]heptan-2-yl)pyrimidin-5- yl)oxy)-N-isopropylbenzamide |
| 29 | 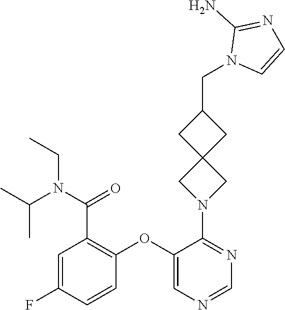 [SMILES: CCN(C(=O)c1cc(F)ccc1Oc1cncnc1N1CC2(CC(Cn3ccnc3N)C2)C1)C(C)C] | 2-((4-(6-((2-Amino-1H-imidazol-1- yl)methyl)-2-azaspiro[3.3]heptan-2- yl)pyrimidin-5-yl)oxy)-N-ethyl-5- fluoro-N-isopropylbenzamide |
| 30 | 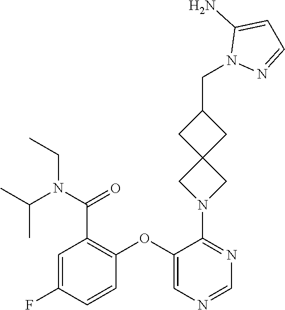 [SMILES: CCN(C(=O)c1cc(F)ccc1Oc1cncnc1N1CC2(CC(Cn3nccc3N)C2)C1)C(C)C] | 2-((4-(6-((5-Amino-1H-pyrazol-1- yl)methyl)-2-azaspiro[3.3]heptan-2- yl)pyrimidin-5-yl)oxy)-N-ethyl-5- fluoro-N-isopropylbenzamide |
| 31 | 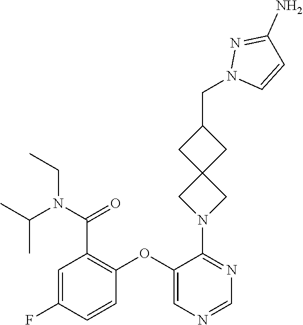 [SMILES: CCN(C(=O)c1cc(F)ccc1Oc1cncnc1N1CC2(CC(Cn3ccc(N)n3)C2)C1)C(C)C] | 2-((4-(6-((3-Amino-1H-pyrazol-1- yl)methyl)-2-azaspiro[3.3]heptan-2- yl)pyrimidin-5-yl)oxy)-N-ethyl-5- fluoro-N-isopropylbenzamide |
| 32 | 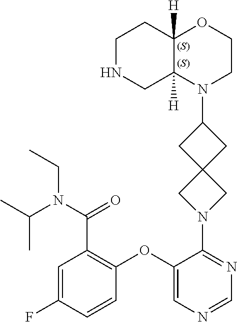 | N-Ethyl-5-fluoro-N-isopropyl-2-((4- (6-((4aS,8aS)-octahydro-4H- pyrido[4,3-b][1,4]oxazin-4-yl)-2- azaspiro[3.3]heptan-2-yl)pyrimidin-5- yl)oxy)benzamide |
| 33 | 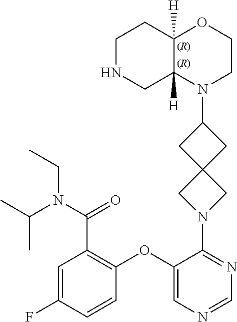 | N-Ethyl-5-fluoro-N-isopropyl-2-((4- (6-((4aR,8aR)-octahydro-4H- pyrido[4,3-b][1,4]oxazin-4-yl)-2- azaspiro[3.3]heptan-2-yl)pyrimidin-5- yl)oxy)benzamide |
| 34 | 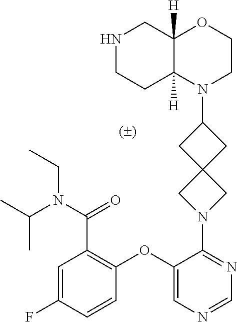 [SMILES: CCN(C(=O)c1cc(F)ccc1Oc1cncnc1N1CC2(CC(N3CCO[C@H]4CNCC[C@@H]43)C2)C1)C(C)C] | (±)-N-Ethyl-5-fluoro-N-isopropyl-2- ((4-(6-((trans)-octahydro-1H- pyrido[3,4-b][1,4]oxazin-1-yl)-2- azaspiro[3.3]heptan-2-yl)pyrimidin-5- yl)oxy)benzamide |
| 35 | 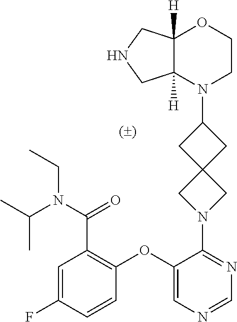 [SMILES: CCN(C(=O)c1cc(F)ccc1Oc1cncnc1N1CC2(CC(N3CCO[C@H]4CNC[C@@H]43)C2)C1)C(C)C] | (±)-N-Ethyl-5-fluoro-2-((4-(6-((trans)- hexahydropyrrolo[3,4-b][1,4]oxazin- 4(4aH)-yl)-2-azaspiro[3.3]heptan-2- yl)pyrimidin-5-yl)oxy)-N- isopropylbenzamide |
| 36 | 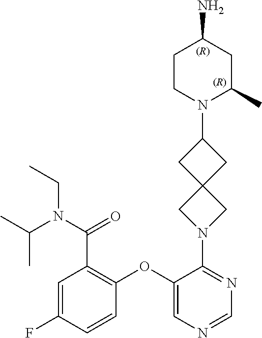 | 2-((4-(6-((2R,4R)-4-Amino-2- methylpiperidin-1-yl)-2- azaspiro[3.3]heptan-2-yl)pyrimidin-5- yl)oxy)-N-ethyl-5-fluoro-N- isopropylbenzamide |
| 37 | 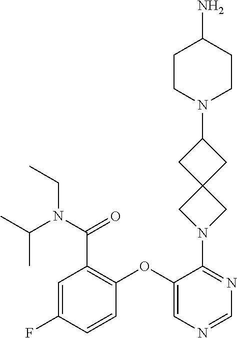 [SMILES: CCN(C(=O)c1cc(F)ccc1Oc1cncnc1N1CC2(CC(N3CCC(N)CC3)C2)C1)C(C)C] | 2-((4-(6-(4-Aminopiperidin-1-yl)-2- azaspiro[3.3]heptan-2-yl)pyrimidin-5- yl)oxy)-N-ethyl-5-fluoro-N- isopropylbenzamide |
| 38 | 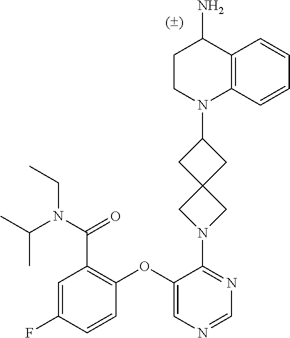 [SMILES: CCN(C(=O)c1cc(F)ccc1Oc1cncnc1N1CC2(CC(N3CCC(N)c4ccccc43)C2)C1)C(C)C] | (±)-2-((4-(6-(4-Amino-3,4- dihydroquinolin-1(2H)-yl)-2- azaspiro[3.3]heptan-2-yl)pyrimidin-5- yl)oxy)-N-ethyl-5-fluoro-N- isopropylbenzamide |
| 39 | 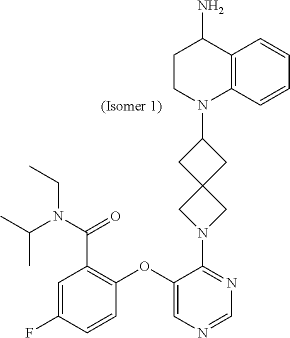 [SMILES: CCN(C(=O)c1cc(F)ccc1Oc1cncnc1N1CC2(CC(N3CCC(N)c4ccccc43)C2)C1)C(C)C] | (E1)-2-((4-(6-(4-Amino-3,4- dihydroquinolin-1(2H)-yl)-2- azaspiro[3.3]heptan-2-yl)pyrimidin-5- yl)oxy)-N-ethyl-5-fluoro-N- isopropylbenzamide |
| 40 | 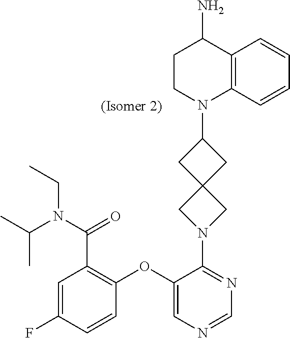 [SMILES: CCN(C(=O)c1cc(F)ccc1Oc1cncnc1N1CC2(CC(N3CCC(N)c4ccccc43)C2)C1)C(C)C] | (E2)-2-((4-(6-(4-Amino-3,4- dihydroquinolin-1(2H)-yl)-2- azaspiro[3.3]heptan-2-yl)pyrimidin-5- yl)oxy)-N-ethyl-5-fluoro-N- isopropylbenzamide |
| 41 | 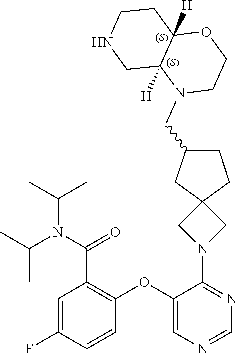 | 5-Fluoro-N,N-diisopropyl-2-((4-(6- (((4aS,8aS)-octahydro-4H-pyrido[4,3- b][1,4]oxazin-4-yl)methyl)-2- azaspiro[3.4]octan-2-yl)pyrimidin-5- yl)oxy)benzamide |
| 42 | 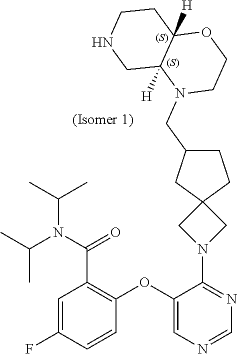 | (D1)-5-Fluoro-N,N-diisopropyl-2-((4- (6-(((4aS,8aS)-octahydro-4H- pyrido[4,3-b][1,4]oxazin-4- yl)methyl)-2-azaspiro[3.4]octan-2- yl)pyrimidin-5-yl)oxy)benzamide |
| 43 | 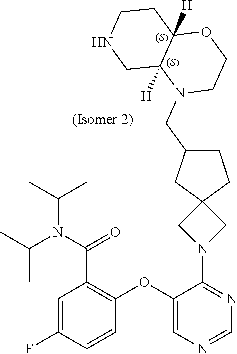 | (D2)-5-Fluoro-N,N-diisopropyl-2-((4- (6-(((4aS,8aS)-octahydro-4H- pyrido[4,3-b][1,4]oxazin-4- yl)methyl)-2-azaspiro[3.4]octan-2- yl)pyrimidin-5-yl)oxy)benzamide |
| 44 | 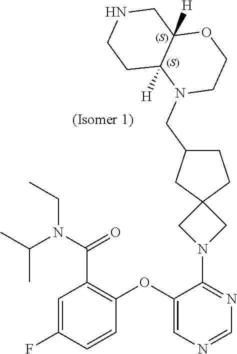 | (D1)-N-Ethyl-5-fluoro-N-isopropyl-2- ((4-(6-(((4aS,8aS)-octahydro-1H- pyrido[3,4-b][1,4]oxazin-1- yl)methyl)-2-azaspiro[3.4]octan-2- yl)pyrimidin-5-yl)oxy)benzamide |
| 45 | 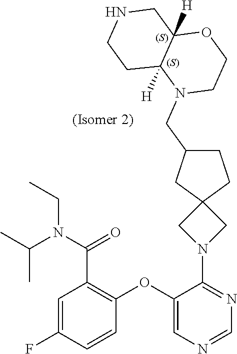 | (D2)-N-Ethyl-5-fluoro-N-isopropyl-2- ((4-(6-(((4aS,8aS)-octahydro-1H- pyrido[3,4-b][1,4]oxazin-1- yl)methyl)-2-azaspiro[3.4]octan-2- yl)pyrimidin-5-yl)oxy)benzamide |
| 46 | 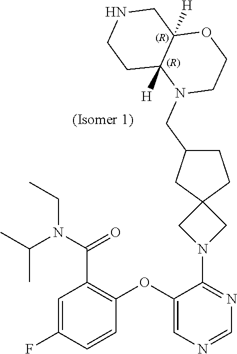 | (D1)-N-Ethyl-5-fluoro-N-isopropyl-2- ((4-(6-(((4aR,8aR)-octahydro-1H- pyrido[3,4-b][1,4]oxazin-1- yl)methyl)-2-azaspiro[3.4]octan-2- yl)pyrimidin-5-yl)oxy)benzamide |
| 47 | 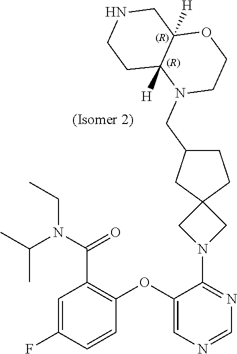 | (D2)-N-Ethyl-5-fluoro-N-isopropyl-2- ((4-(6-(((4aR,8aR)-octahydro-1H- pyrido[3,4-b][1,4]oxazin-1- yl)methyl)-2-azaspiro[3.4]octan-2- yl)pyrimidin-5-yl)oxy)benzamide |
| 48 | 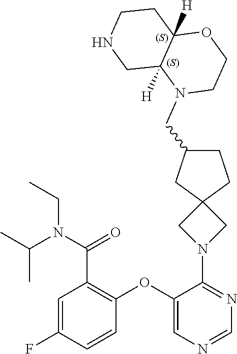 | N-Ethyl-5-fluoro-N-isopropyl-2-((4- (6-(((4aS,8aS)-octahydro-4H- pyrido[4,3-b][1,4]oxazin-4- yl)methyl)-2-azaspiro[3.4]octan-2- yl)pyrimidin-5-yl)oxy)benzamide |
| 49 | 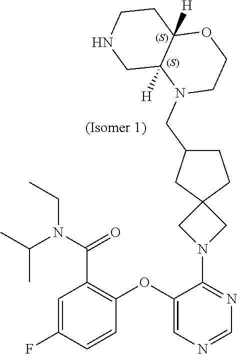 | (D1)-N-Ethyl-5-fluoro-N-isopropyl-2- ((4-(6-(((4aS,8aS)-octahydro-4H- pyrido[4,3-b][1,4]oxazin-4- yl)methyl)-2-azaspiro[3.4]octan-2- yl)pyrimidin-5-yl)oxy)benzamide |
| 50 | 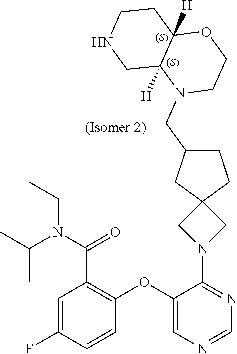 | (D2)-N-Ethyl-5-fluoro-N-isopropyl-2- ((4-(6-(((4aS,8aS)-octahydro-4H- pyrido[4,3-b][1,4]oxazin-4- yl)methyl)-2-azaspiro[3.4]octan-2- yl)pyrimidin-5-yl)oxy)benzamide |
| 51 | 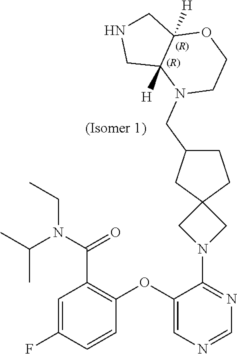 | (D1)-N-Ethyl-5-fluoro-2-((4-(6- (((4aR,7aR)-hexahydropyrrolo[3,4- b][1,4]oxazin-4(4aH)-yl)methyl)-2- azaspiro[3.4]octan-2-yl)pyrimidin-5- yl)oxy)-N-isopropylbenzamide |
| 52 | 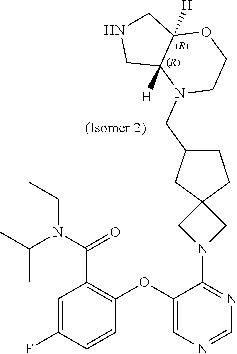 | (D2)-N-Ethyl-5-fluoro-2-((4-(6- (((4aR,7aR)-hexahydropyrrolo[3,4- b][1,4]oxazin-4(4aH)-yl)methyl)-2- azaspiro[3.4]octan-2-yl)pyrimidin-5- yl)oxy)-N-isopropylbenzamide |
| 53 | 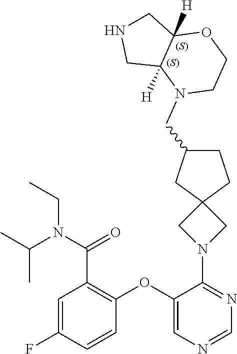 | N-Ethyl-5-fluoro-2-((4-(6-(((4aS,7aS)- hexahydropyrrolo[3,4-b][1,4]oxazin- 4(4aH)-yl)methyl)-2- azaspiro[3.4]octan-2-yl)pyrimidin-5- yl)oxy)-N-isopropylbenzamide |
| 54 | 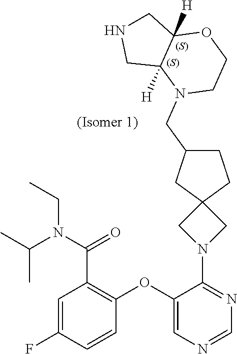 | (D1)-N-Ethyl-5-fluoro-2-((4-(6- (((4aS,7aS)-hexahydropyrrolo[3,4- b][1,4]oxazin-4(4aH)-yl)methyl)-2- azaspiro[3.4]octan-2-yl)pyrimidin-5- yl)oxy)-N-isopropylbenzamide |
| 55 | 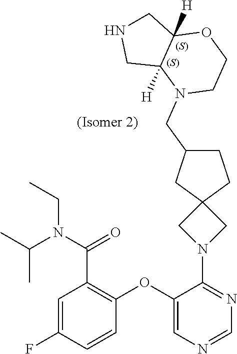 | (D2)-N-Ethyl-5-fluoro-2-((4-(6- (((4aS,7aS)-hexahydropyrrolo[3,4- b][1,4]oxazin-4(4aH)-yl)methyl)-2- azaspiro[3.4]octan-2-yl)pyrimidin-5- yl)oxy)-N-isopropylbenzamide |
| 56 | 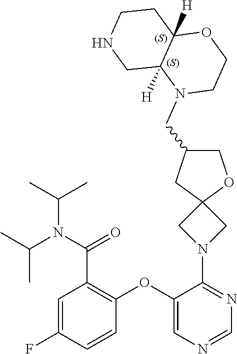 | 5-Fluoro-N,N-diisopropyl-2-((4-(7- (((4aS,8aS)-octahydro-4H-pyrido[4,3- b][1,4]oxazin-4-yl)methyl)-5-oxa-2- azaspiro[3.4]octan-2-yl)pyrimidin-5- yl)oxy)benzamide |
| 57 | 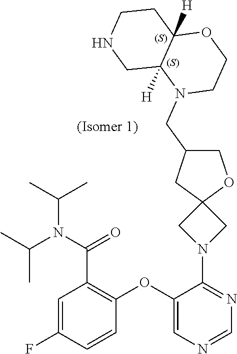 | (D1)-5-Fluoro-N,N-Diisopropyl-2-((4- (7-(((4aS,8aS)-octahydro-4H- pyrido[4,3-b][1,4]oxazin-4- yl)methyl)-5-oxa-2- azaspiro[3.4]octan-2-yl)pyrimidin-5- yl)oxy)benzamide |
| 58 | 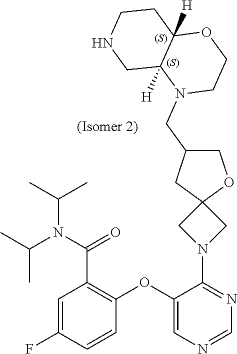 | (D2)-5-Fluoro-N,N-diisopropyl-2-((4- (7-(((4aS,8aS)-octahydro-4H- pyrido[4,3-b][1,4]oxazin-4- yl)methyl)-5-oxa-2- azaspiro[3.4]octan-2-yl)pyrimidin-5- yl)oxy)benzamide |
| 59 | 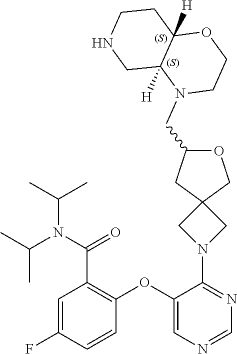 | 5-Fluoro-N,N-diisopropyl-2-((4-(7- (((4aS,8aS)-octahydro-4H-pyrido[4,3- b][1,4]oxazin-4-yl)methyl)-6-oxa-2- azaspiro[3.4]octan-2-yl)pyrimidin-5- yl)oxy)benzamide |
| 59-a | 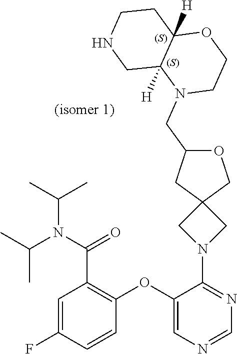 | (D1)-5-Fluoro-N,N-diisopropyl-2-((4- (7-(((4aS,8aS)-octahydro-4H- pyrido[4,3-b][1,4]oxazin-4- yl)methyl)-6-oxa-2- azaspiro[3.4]octan-2-yl)pyrimidin-5- yl)oxy)benzamide |
| 59-b | 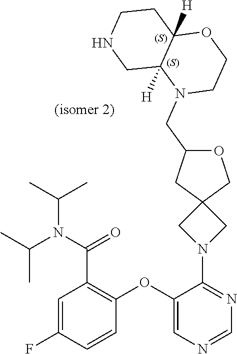 | (D2)-5-Fluoro-N,N-diisopropyl-2-((4- (7-(((4aS,8aS)-octahydro-4H- pyrido[4,3-b][1,4]oxazin-4- yl)methyl)-6-oxa-2- azaspiro[3.4]octan-2-yl)pyrimidin-5- yl)oxy)benzamide |
| 60 | 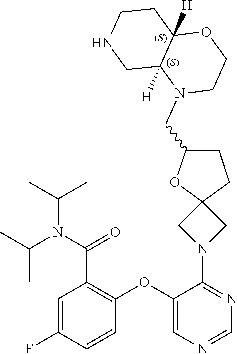 | 5-Fluoro-N,N-diisopropyl-2-((4-(6- (((4aS,8aS)-octahydro-4H-pyrido[4,3- b][1,4]oxazin-4-yl)methyl)-5-oxa-2- azaspiro[3.4]octan-2-yl)pyrimidin-5- yl)oxy)benzamide |
| 60-a | 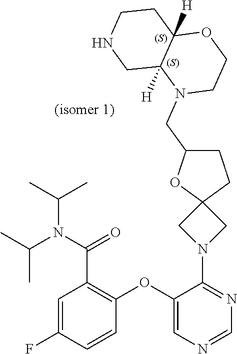 | (D1)-5-Fluoro-N,N-diisopropyl-2-((4- (6-(((4aS,8aS)-octahydro-4H- pyrido[4,3-b][1,4]oxazin-4- yl)methyl)-5-oxa-2- azaspiro[3.4]octan-2-yl)pyrimidin-5- yl)oxy)benzamide |
| 60-b | 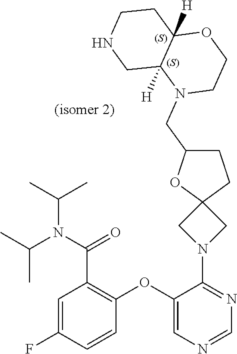 | (D2)-5-Fluoro-N,N-diisopropyl-2-((4- (6-(((4aS,8aS)-octahydro-4H- pyrido[4,3-b][1,4]oxazin-4- yl)methyl)-5-oxa-2- azaspiro[3.4]octan-2-yl)pyrimidin-5- yl)oxy)benzamide |
| 61 | 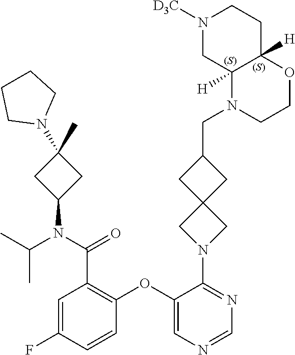 | 5-Fluoro-N-isopropyl-N-((1r,3r)-3- methyl-3-(pyrrolidin-1-yl)cyclobutyl)- 2-((4-(6-(((4aS,8aS)-6-(methyl- d³)octahydro-4H-pyrido[4,3- b][1,4]oxazin-4-yl)methyl)-2- azaspiro[3.3]heptan-2-yl)pyrimidin-5- yl)oxy)benzamide |
| 62 | 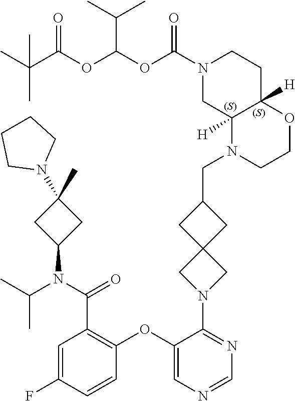 | 2-Methyl-1-(pivaloyloxy)propyl (4aS,8aS)-4-((2-(5-(4-fluoro-2- (isopropyl((1r,3r)-3-methyl-3- (pyrrolidin-1-yl)cyclobutyl) carbamoyl)phenoxy)pyrimidin- 4-yl)-2-azaspiro[3.3]heptan-6- yl)methyl)hexahydro-2H-pyrido[4,3- b][1,4]oxazine-6(5H)-carboxylate |
| 63 | 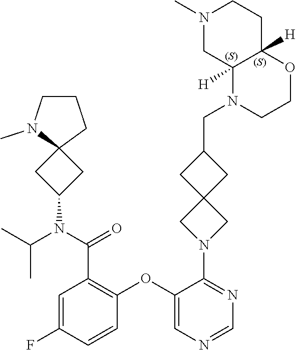 | 5-Fluoro-N-isopropyl-N-((2s,4r)-5- methyl-5-azaspiro[3.4]octan-2-yl)-2- ((4-(6-(((4aS,8aS)-6-methyloctahydro- 4H-pyrido[4,3-b][1,4]oxazin-4- yl)methyl)-2-azaspiro[3.3]heptan-2- yl)pyrimidin-5-yl)oxy)benzamide |
| 64 | 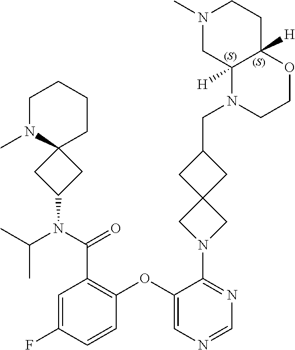 | 5-Fluoro-N-isopropyl-N-((2s,4r)-5- methyl-5-azaspiro[3.5]nonan-2-yl)-2- ((4-(6-(((4aS,8aS)-6-methyloctahydro- 4H-pyrido[4,3-b][1,4]oxazin-4- yl)methyl)-2-azaspiro[3.3]heptan-2- yl)pyrimidin-5-yl)oxy)benzamide |
| 65 | 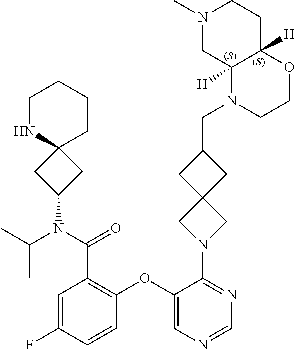 | 5-Fluoro-N-isopropyl-2-((4-(6- (((4aS,8aS)-6-methyloctahydro-4H- pyrido[4,3-b][1,4]oxazin-4- yl)methyl)-2-azaspiro[3.3]heptan-2- yl)pyrimidin-5-yl)oxy)-N-((2s,4r)-5- azaspiro[3.5]nonan-2-yl)benzamide |

In some embodiments, the present disclosure is directed to a compound as shown in Table 1 or a stereoisomer thereof, or a pharmaceutically acceptable salt thereof.

In some embodiments, the present disclosure is directed to a compound as shown in Table 1 or a pharmaceutically acceptable salt thereof.

In some embodiments, the present disclosure is directed to a compound as shown in Table 1.

In some embodiments, the present disclosure is directed to a compound as shown in Table 1 or a pharmaceutically acceptable salt thereof, wherein the salt is hydrochloride.

In some embodiments, a compound according to any embodiments herein (e.g., Formulae I, II, II-a, II-b, II-c, II-d, and II-e, and Table 1) exhibits an inhibition activity against the binding of menin and MLL. In some embodiments, a compound according to any embodiments herein (e.g., Formulae I, II, II-a, II-b, II-c, II-d, and II-e, and Table 1) exhibits an inhibition activity against the binding of menin and MLL. In some embodiments, a compound according to any embodiments herein e.g., Formulae I, II, II-a, II-b, II-c, II-d, and II-e, and Table 1) exhibits an inhibition activity against the binding of menin and MLL which is useful in the treatment and/or prevention of one or more diseases in which menin and MLL play a role.

In some embodiments, a compound according to any embodiments herein (e.g., Formulae I, II, II-a, II-b, II-c, II-d, and II-e, and Table 1) exhibits low hERG binding. In some embodiments, a compound according to any embodiments herein (e.g., Formulae I, II, II-a, II-b, II-c, II-d, and II-e, and Table 1) minimizes hERG binding and is useful for the treatment of one or more diseases in which menin and MLL play a role. Without being bound to any theory, one of the primary causes of QT prolongation is thought to be blockage of the hERG potassium channel in cardiac myocytes. In some embodiments, the compounds of the present disclosure (e.g., Formulae I, II, II-a, II-b, II-c, II-d, and II-e, and Table 1) do not significantly block the hERG potassium channel.

In some embodiments, the compounds of the present disclosure (e.g., Formulae I, II, II-a, II-b, II-c, II-d, and II-e, and Table 1) do not significantly block the hERG potassium channel (e.g., an IC₅₀ greater than 1 μM, 5 μM, 10 μM, 15 μM, 20 μM, 25 μM, 30 μM, 35 μM, 40 μM, or 50 μM) as measured by a standard patch clamp hERG assay.

In some embodiments, the compounds of the present disclosure (e.g., Formulae I, II, II-a, II-b, II-c, II-d, and II-e, and Table 1) do not significantly block the hERG potassium channel (e.g., a compound of the invention has an IC₅₀ greater than 1 μM, 5 μM, 10 μM, 15 μM, 20 μM, 25 μM, 30 μM, 35 μM, 40 μM, or 50 μM for the hERG potassium channel).

In some embodiments and without wishing to be bound to any theory, the present disclosure is directed to inhibitors of the menin-HLL interaction comprising a pyrrolidine moiety (e.g., Formulae I, II, II-a, II-b, II-c, II-d, and II-e, and Table 1) where the pyrrolidine moiety has been found to reduce hERG inhibition.

In some embodiments and without wishing to be bound to any theory, the present disclosure is directed to inhibitors of the menin-HLL interaction comprising a piperidine moiety (e.g., Formulae I, II, II-a, II-b, II-c, II-d, and II-e, and Table 1) where the piperidine moiety has been found to reduce hERG inhibition.

In some embodiments and without wishing to be bound to any theory, the present disclosure is directed to inhibitors of the menin-HLL interaction (e.g., Formulae I, II, II-a, II-b, II-c, II-d, and II-e, and Table 1) which are resistant to metabolism. In some embodiments and without wishing to be bound to any theory, the present disclosure is directed to inhibitors of the menin-MLL interaction (e.g., Formulae I, II, II-a, II-b, II-c, II-d, and II-e, and Table 1) which are resistant to metabolism, where the metabolites are inhibitors of the hERG potassium channel. In some embodiments and without wishing to be bound to any theory, the present disclosure is directed to inhibitors of the menin-HLL interaction (e.g., Formulae I, II, II-a, II-b, II-c, II-d, and II-e, and Table 1) which are resistant to metabolism, where the metabolism decreases the bioavailability of the inhibitor. In some embodiments and without wishing to be bound to any theory, the present disclosure is directed to inhibitors of the menin-HLL interaction (e.g., Formulae I, II, II-a, II-b, II-c, II-d, and II-e, and Table 1) which are resistant to metabolism, where the metabolism decreases the bioavailability of the inhibitor and the corresponding metabolites are more effective (e.g., by IC₅₀, etc.) at binding the hERG potassium channel.

In some embodiments, one or more hydrogen atoms in any of the compounds of Formula I, II, II-a, II-b, II-c, II-d, or II-e may be replaced with one or more deuterium atoms.

Another aspect is an isotopically labeled compound of any of the formulae delineated herein. Such compounds have one or more isotopic atoms (e.g., ³H, ²H, ¹⁴C, ¹³C, ¹⁸F, ³⁵S, ³²P, ¹²⁵I, and ¹³¹I) introduced into the compound. Such compounds are useful for drug metabolism studies and diagnostics, as well as therapeutic applications. In some embodiments, the compound is an isotopic derivative of any one of the compounds described in Table 1, or a pharmaceutically acceptable salt thereof.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the present disclosure or a pharmaceutically acceptable salt or solvate thereof that contains the aforementioned deuterium atom(s) is within the scope of the disclosure.

Further, substitution with deuterium (i.e., 2H) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

Potency can also be determined by IC₅₀ value. A compound with a lower IC₅₀ value, as determined under substantially similar conditions, is more potent relative to a compound with a higher IC₅₀ value.

Potency can also be determined by EC₅₀ value. A compound with a lower EC₅₀ value, as determined under substantially similar conditions, is more potent relative to a compound with a higher EC₅₀ value.

In some embodiments, a pair of enantiomers have similar potency.

In some embodiments, a pair of enantiomers have different potency. In specific embodiments, the enantiomer with (R) configuration is more potent than the enantiomer with (S) configuration. In other specific embodiments, the enantiomer with (S) configuration is more potent than the enantiomer with (R) configuration. For example, Compound No. 26 is more potent than Compound No. 27.

In some embodiments, a pair of isomers have similar potency.

In some embodiments, a pair of isomers have different potency. In specific embodiments, the trans isomer is more potent than the cis isomer. In specific embodiments, the cis isomer is more potent than the trans isomer.

In some embodiments, a group of diastereomers have similar potency.

In some embodiments, a group of diastereomers have different potency.

In specific embodiments, the diastereomer with (R)(R) configuration is the most potent among the group of diastereomers.

In specific embodiments, the diastereomer with (S)(S) configuration is the most potent among the group of diastereomers.

In specific embodiments, the diastereomer with (S)(R) configuration is the most potent among the group of diastereomers.

In specific embodiments, the diastereomer with (R)(S) configuration is the most potent among the group of diastereomers.

In specific embodiments, the diastereomer with (R)(R) configuration is more potent than the diastereomer with (S)(S) configuration.

In specific embodiments, the diastereomer with (S)(S) configuration is more potent than the diastereomer with (R)(R) configuration. For example, Compound No. 1 is more potent than Compound No. 4 and Compound No. 32 is more potent than Compound No. 33.

In specific embodiments, the diastereomer with (R)(S) configuration is more potent than the diastereomer with (S)(R) configuration.

In specific embodiments, the diastereomer with (S)(R) configuration is more potent than the diastereomer with (R)(S) configuration.

The compounds of the application are defined herein by their chemical structures and/or chemical names. Where a compound is referred to by both a chemical structure and a chemical name, and the chemical structure and chemical name conflict, the chemical structure is determinative of the compound's identity.

In another aspect, the application provides a method of synthesizing a compound disclosed herein. The synthesis of the compounds of the application can be found herein and in the Examples below. Other embodiments are a method of making a compound of any of the formulae herein using any one, or combination of, reactions delineated herein. The method can include the use of one or more intermediates or chemical reagents delineated herein.

In some embodiments, each of the intermediates prepared in the Schemes herein are considered embodiments of the present disclosure. In some embodiments, each of the intermediates prepared in the Examples herein are considered embodiments of the present disclosure. In some embodiments, each synthetic step as disclosed in the Schemes herein is separately considered as part of the present disclosure. In some embodiments, each synthetic step as disclosed in the Examples herein is separately considered as part of the present disclosure.

It is appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various cycloalkyl, and heterocyclyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

For compounds of the disclosure in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups.

The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the term “amino,” employed alone or in combination with other terms, refers to a group of formula —NH₂. In some embodiments, an amine can be substituted by one or more groups, e.g., —N—(C₁-C₆ alkyl)₂. In some embodiments, when two groups are attached to an amine they can be the same or different. Each group is selected independently of each other and can be each independently optionally substituted.

As used herein, the term “halogen” or “halo,” employed alone or in combination with other terms, refers to a halogen atom selected from F, Cl, I or Br. In some embodiments, “halo” refers to a halogen atom selected from F, Cl, or Br. In some embodiments, the halo substituent is F.

The term “haloalkyl” or “haloalkoxyl” refers to an alkyl or alkoxyl substituted with one or more halogen atoms. In some embodiments, the haloalkyl group is fluorinated. In some embodiments, the haloalkyl group is fluorinated only. In some embodiments, the haloalkyl group is fluoromethyl, difluoromethyl, or trifluoromethyl. In some embodiments, the haloalkyl group is trifluoromethyl. In some embodiments, the haloalkyl group is 2,2,2-trifluoroethyl. In some embodiments, the haloalkyl group is 2,2-difluoroethyl. In some embodiments, the haloalkyl group has 1 to 6 or 1 to 4 carbon atoms.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups.

In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic. In some embodiments, cycloalkyl may optionally contain one or more alkenylene groups as part of the ring structure. Cycloalkyl groups can include mono- or polycyclic ring systems. Polycyclic ring systems can include fused ring systems and spirocycles. Also included in the definition of cycloalkyl are moieties that have one or more aromatic rings fused (i.e., having a bond in common with) to the cycloalkyl ring, for example, benzo or pyrido derivatives of cyclopentane, cyclopentene, cyclohexane, and the like. A heterocyclyl group that includes a fused aromatic (e.g., aryl or heteroaryl) moiety can be attached to the molecule through an atom from either the aromatic or non-aromatic portion. One or more ring-forming carbon atoms of a cycloalkyl group can be oxidized to form carbonyl linkages. In some embodiments, cycloalkyl is C₃₋₁₀ cycloalkyl, C₃₋₇ cycloalkyl, or C₅₋₆ cycloalkyl. Exemplary cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. Further exemplary cycloalkyl groups include cyclopropyl, cyclobutyl, cyclopentyl, and cyclohexyl. Additional example cycloalkyl groups, where the cycloalkyl group has a fused aryl or heteroaryl moiety, include tetrahydronaphthalen-2-yl, 2,3-dihydro-1H-inden-2-yl; 2,3,4,9-tetrahydro-1H-carbazol-7-yl; 2,6,7,8-tetrahydrobenzo[cd]indazol-4-yl; and 5,6,7,8,9,10-hexahydrocyclohepta[b]indol-3-yl. Further exemplary cycloalkyl groups include

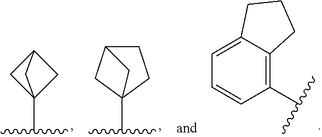

As used herein, the term “aryl,” employed alone or in combination with other terms, refers to a monocyclic or polycyclic (e.g., having 2, 3 or 4 fused rings) aromatic hydrocarbon, such as, but not limited to, phenyl, 1-naphthyl, 2-naphthyl, anthracenyl, phenanthrenyl, and the like. In some embodiments, aryl is C₆₋₁₀ aryl. In some embodiments, aryl is C₆₋₁₄ aryl. In some embodiments, the aryl group is a naphthalene ring or phenyl ring. In some embodiments, the aryl group is phenyl.

As used herein, the term “heteroaryl,” employed alone or in combination with other terms, refers to a monocyclic or polycyclic (e.g., having 2, 3 or 4 fused rings) aromatic heterocylic moiety, having one or more heteroatom ring members selected from nitrogen, sulfur and oxygen. In some embodiments, the heteroaryl group has 1, 2, 3, or 4 heteroatom ring members. In some embodiments, the heteroaryl group has 1, 2, or 3 heteroatom ring members. In some embodiments, the heteroaryl group has 1 or 2 heteroatom ring members. In some embodiments, the heteroaryl group has 1 heteroatom ring member. In some embodiments, the heteroaryl group is 5- to 10-membered or 5- to 6-membered. In some embodiments, the heteroaryl group is 5-membered. In some embodiments, the heteroaryl group is 6-membered. In some embodiments, the heteroaryl group is 9- or 10-membered bicyclic. In some embodiments, the heteroaryl is 9-member bicyclic.

When the heteroaryl group contains more than one heteroatom ring member, the heteroatoms may be the same or different. The nitrogen atoms in the ring(s) of the heteroaryl group can be oxidized to form N-oxides. Example heteroaryl groups include, but are not limited to, pyridinyl, pyrimidinyl, pyrazinyl, pyridazinyl, pyrrolyl, pyrazolyl, azolyl, oxazolyl, isoxazolyl, thiazolyl, isothiazolyl, imidazolyl, furanyl, thiophenyl, triazolyl, tetrazolyl, thiadiazolyl, quinolinyl, isoquinolinyl, indolyl, benzothiopheneyl, benzofuranyl, benzisoxazolyl, benzoimidazolyl, imidazo[1,2-b]thiazolyl, purinyl, triazinyl, and the like. In some embodiments, the heteroaryl group is 9H-carbazol-2-yl; 1H-benzo[d]imidazol-6-yl; 1H-indol-6-yl; 1H-indazol-6-yl; 2H-indazol-4-yl; 1H-benzo[d][1,2,3]triazol-6-yl; benzo[d]oxazol-2-yl; quinolin-6-yl; or benzo[d]thiazol-2-yl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the phrase “optionally substituted” means unsubstituted or substituted. As used herein, the term “substituted” or “substituted with one or more”, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “heterocyclyl” or “heterocycloalkyl” employed alone or in combination with other terms, refers to a non-aromatic heterocyclic ring system, which may optionally contain one or more unsaturations as part of the ring structure, and which has at least one heteroatom ring member independently selected from nitrogen, sulfur and oxygen. In some embodiments, the heterocyclyl group has 1, 2, 3, or 4 heteroatom ring members. In some embodiments, the heterocyclyl group has 1, 2, or 3 heteroatom ring members. In some embodiments, the heterocyclyl group has 1 or 2 heteroatom ring members. In some embodiments, the heterocyclyl group has 1 heteroatom ring member. When the heterocyclyl group contains more than one heteroatom in the ring, the heteroatoms may be the same or different. Example ring-forming members include CH, CH₂, C(O), N, NH, O, S, S(O), and S(═O)₂. Heterocyclyl groups can include mono- or polycyclic (e.g., having 2, 3 or 4 fused rings) ring systems. Polycyclic rings can include both fused systems and spirocycles. Also included in the definition of heterocyclyl are moieties that have one or more aromatic rings fused (i.e., having a bond in common with) to the non-aromatic ring, for example, 1, 2, 3, 4-tetrahydro-quinoline, dihydrobenzofuran and the like. A heterocyclyl group that includes a fused aromatic moiety can be attached to the molecule through an atom from either the aromatic or non-aromatic portion. The carbon atoms or heteroatoms in the ring(s) of the heterocyclyl group can be oxidized to form a carbonyl, sulfinyl, or sulfonyl group (or other oxidized linkage) or a nitrogen atom can be quaternized. In some embodiments, heterocyclyl is 5- to 10-membered, 4- to 10-membered, 4- to 7-membered, 5-membered, or 6-membered. Examples of heterocyclyl groups include 1, 2, 3, 4-tetrahydro-quinolinyl, dihydrobenzofuranyl, azetidinyl, azepanyl, pyrrolidinyl, piperidinyl, piperazinyl, morpholinyl, thiomorpholinyl, and pyranyl. Examples of heterocyclyl groups that include one or more fused aromatic groups (e.g., aryl or heteroaryl) include N-(2′-oxospiro[cyclohexane-1,3′-indolin]-6′-yl; 1,2,3,4-tetrahydroisoquinolin-6-yl; 2,3-dihydro-1H-benzo[d]imidazol-5-yl; 1,3-dihydrospiro[indene-2,3′-indolin]-6′-yl; 2,3-dihydrobenzo[d]oxazol-5-yl; 1,2-dihydroquinolin-7-yl; indolin-6-yl; spiro[cyclopentane-1,3′-indolin]-6′-yl; spiro[cyclohexane-1,3′-indolin]-6′-yl; chroman-6-yl; 3,4-dihydro-2H-benzo[b][1,4]oxazin-6-yl; and benzo[d][1,3]dioxol-5-yl. Examples of “heterocyclyl” or “heterocycloalkyl” groups include

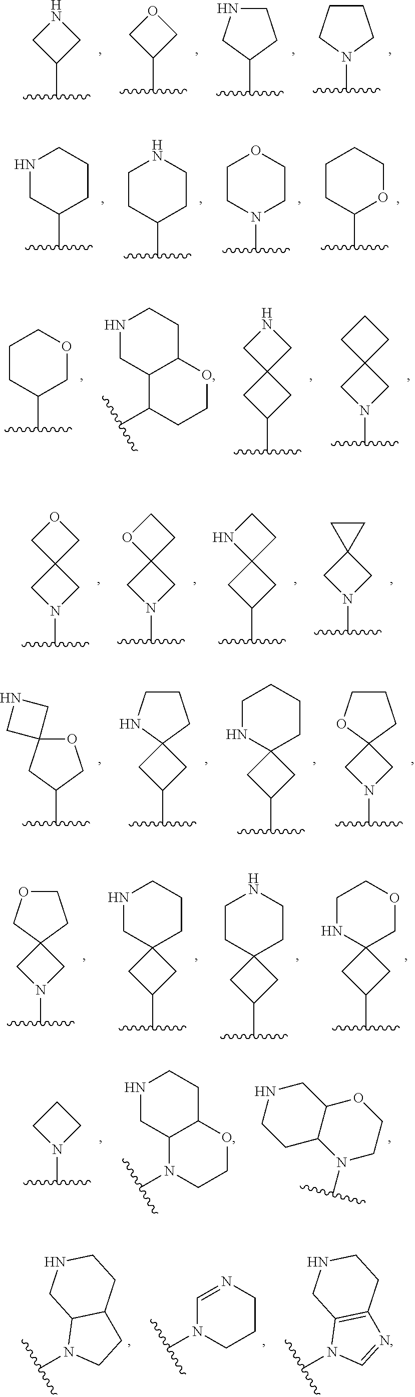

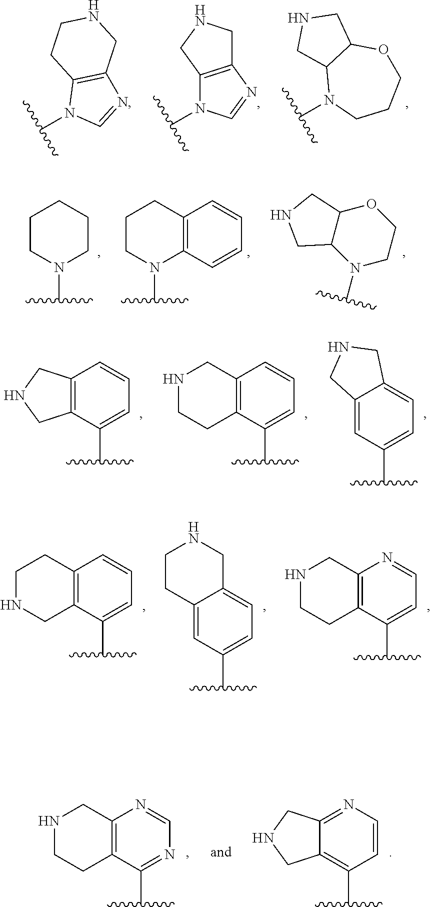

As used herein, the term “alkylene” refers to a refers to a divalent radical derived from an alkane, as exemplified, by (—CH₂—)ₙ, wherein n may be 1 to about 24. By way of example only, such groups include, but are not limited to, groups having 10 or fewer carbon atoms such as the structures —CH₂CH₂— and —CH₂CH₂CH₂CH₂—.

As used herein, the terms “Cₙ-Cₘ” and “Cₙ₋ₘ”, where each of n and m is an integer, can be used interchangeably.

As used herein, the term “cycloalkylene” refers to a cycloalkyl group wherein two hydrogens are removed to provide a divalent radical. It is understood that the two hydrogens, prior to removal, can be attached to same or different atoms in the cycloalkyl group. Examples of cycloalkylene groups include

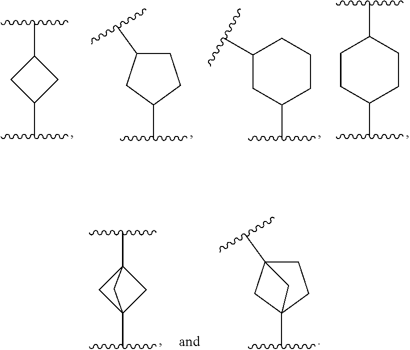

As used herein, “ [symbol] ” represents a bond to the position indicated by the corresponding variable, for example Ring A, R¹, R², R³, G, X, A, B, D, E, Z, W, Y, V, R^{g}, R^{1s}, R^{1as}, R^{1bs}, R^{1cs}, R^{3a}, R^{4a}, R^{4b}, R^{3a}′, R^{3b}′, R^{3c}′, R^{4a}′ or R^{4b}′.

As used in the embodiments describing Ring A, it is intended that the position of the substituents can be at any position allowed by the available valences according to general formulae I, II, II-a, II-b, II-c, II-d, and II-e, or a stereoisomer, or a pharmaceutically acceptable salt thereof.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R and S sequencing rules of Cahn and Prelog, or by the manner in which the molecule rotates the plane of polarised light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture”. The compounds described herein can be asymmetric (e.g., having one or more stereocenters). All stereoisomers, such as enantiomers and diastereoisomers, are intended unless otherwise indicated. Where a compound name or structure is silent with respect to the stereochemistry of a stereocenter, all possible configurations at the stereocenter are intended. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

When the compounds of the disclosure contain a chiral center, the compounds can be any of the possible stereoisomers. In compounds with a single chiral center, the stereochemistry of the chiral center can be (R) or (S). In compounds with two chiral centers, the stereochemistry of the chiral centers can each be independently (R) or (S) so the configuration of the chiral centers can be (R) and (R), (R) and (S); (S) and (R), or (S) and (S). In compounds with three chiral centers, the stereochemistry each of the three chiral centers can each be independently (R) or (S) so the configuration of the chiral centers can be (R), (R) and (R); (R), (R) and (S); (R), (S) and (R); (R), (S) and (S); (S), (R) and (R); (S), (R) and (S); (S), (S) and (R); or (S), (S) and (S).

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereoisomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

As used herein, the terms “E1” and “E2” correspond to first and second enantiomers to elute from a chiral chromatography column as discussed in the Examples.

As used herein, the terms “D1” and “D2” correspond to first and second diastereomers to elute from a chiral chromatography column as discussed in the Examples.

When a disclosed compound is named or depicted without indicating the stereochemistry of one or more stereocenters, each of the stereoisomers resulting from the possible stereochemistries at the undefined stereocenter(s) are intended to be encompassed. For example, if a stereocenter is not designated as R or S, then either or both are intended.

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the disclosure can also include all isotopes of atoms occurring in the intermediates or final compounds. Isotopes include those atoms having the same atomic number but different mass numbers. Isotopes of constituent atoms of the compounds of the disclosure can be present in natural or non-natural abundance. Examples of isotopes of hydrogen include deuterium and tritium. In some embodiments, the compounds of the disclosure are deuterated, meaning at least one deuterium atom is present in the place of a hydrogen atom. In some embodiments, 1, 2, 3, 4, 5, 6, 7, or 8 hydrogens in a compound of the disclosure are replaced by deuterium. In some embodiments, 1, 2, or 3 hydrogens in a compound of the disclosure are replaced by deuterium. In some embodiments, 3 hydrogens in a compound of the disclosure are replaced by deuterium. Methods for replacing hydrogen with deuterium in a molecule are known in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified (e.g., in the case of purine rings, unless otherwise indicated, when the compound name or structure has the 9H tautomer, it is understood that the 7H tautomer is also encompassed).

All compounds, and pharmaceutically acceptable salts thereof, can be found together with other substances such as water and solvents (e.g., hydrates and solvates) or can be isolated.

It will be understood that the compounds of the present disclosure and any pharmaceutically acceptable salts thereof, comprise stereoisomers, mixtures of stereoisomers, polymorphs of all isomeric forms of said compounds.

In some embodiments, the compounds of the disclosure, or salts thereof, or crystalline forms of any of the aforementioned, are purified or substantially isolated. By “substantially isolated” is meant that the compound is at least partially or substantially separated from the environment in which it was formed or detected. Partial separation can include, for example, a composition enriched in a compound of the disclosure. Substantial separation can include compositions containing at least about 50%, at least about 60%, at least about 70%, at least about 80%, at least about 90%, at least about 95%, at least about 97%, or at least about 99% by weight of the compounds of the disclosure, or salt thereof. In some embodiments, the compounds of the disclosure, or salts thereof, or crystalline forms of any of the aforementioned, can be prepared with a purity of about 75% or more, 80% or more, 85% or more, 90% or more, 95% or more, 98% or more, or 99% or more.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, for example, a temperature from about 20° C. to about 30° C.

The present disclosure also includes pharmaceutically acceptable salts of the compounds described herein. As used herein, “pharmaceutically acceptable salts” or “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred.

The compounds disclosed herein include the compounds themselves, as well as their salts, their solvates, and their prodrugs, if applicable. A salt, for example, can be formed between an anion and a positively charged group (e.g., protonated amino) on a compound of this disclosure.

Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of this disclosure. Suitable cations include sodium ion, potassium ion, magnesium ion, calcium ion, and an ammonium cation such as tetramethylammonium ion. The compounds of this disclosure also include those salts containing quaternary nitrogen atoms. Examples of prodrugs include esters and other pharmaceutically acceptable derivatives, which, upon administration to a subject, are capable of providing active compounds of this disclosure.

Additionally, physiologically acceptable, i.e., pharmaceutically compatible, salts can be salts of the compounds disclosed herein with inorganic or organic acids. Preference is given to salts with inorganic acids, such as, for example, hydrochloric acid, hydrobromic acid, phosphoric acid or sulphuric acid, or to salts with organic carboxylic or sulphonic acids, such as, for example, acetic acid, trifluoroacetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

Other pharmaceutically compatible salts which may be mentioned are salts with customary bases, such as, for example, alkali metal salts (for example sodium or potassium salts), alkaline earth metal salts (for example calcium or magnesium salts) or ammonium salts, derived from ammonia or organic amines, such as, for example, diethylamine, triethylamine, ethyldiisopropylamine, procaine, dibenzylamine, N-methylmorpholine, dihydroabietylamine or methylpiperidine.

As used herein, “pharmaceutically acceptable salts” can refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts can include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, or an alkaline earth metal ion, e.g., an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidine, pyrrolidine, benzylamine, tetramethylammonium hydroxide and the like.

It should be understood that all references to pharmaceutically acceptable salts include solvent addition forms (solvates) or crystal forms (polymorphs) as defined herein, of the same salt.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein.

Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

The compounds of the present disclosure may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at a sulfonylurea group.

Accordingly, the present disclosure includes those compounds of the present disclosure as defined hereinbefore when made available by organic synthesis and when made available within the human or animal body by way of cleavage of a prodrug thereof. Accordingly, the present disclosure includes those compounds of the present disclosure that are produced by organic synthetic means and also such compounds that are produced in the human or animal body by way of metabolism of a precursor compound, that is a compound of the present disclosure may be a synthetically-produced compound or a metabolically-produced compound.

A suitable pharmaceutically acceptable prodrug of a compound of the present disclosure is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of the present disclosure that possesses a hydroxy group is, for example, an in vivo cleavable ester or ether thereof. An in vivo cleavable ester or ether of a compound of the present disclosure containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of the present disclosure that possesses a carboxy group is, for example, an in vivo cleavable amide thereof, for example an amide formed with an amine such as ammonia, a C₁-C₄ alkylamine such as methylamine, a (C₁-C₄ alkyl)₂amine such as dimethylamine, N-ethyl N-methylamine or diethylamine, a C₁-C₄ alkoxy C₂-C₄ alkylamine such as 2 methoxyethylamine, a phenyl C₁-C₄ alkylamine such as benzylamine and amino acids such as glycine or an ester thereof.

A suitable pharmaceutically acceptable prodrug of a compound of the present disclosure that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl, and 4-(C₁-C₄ alkyl)piperazin-1-ylmethyl.

The in vivo effects of a compound of the present disclosure may be exerted in part by one or more metabolites that are formed within the human or animal body after administration of a compound of the present disclosure. As stated hereinbefore, the in vivo effects of a compound of the present disclosure may also be exerted by way of metabolism of a precursor compound (a prodrug).

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Synthesis

In some aspects, the present disclosure provides a method of preparing a compound disclosed herein.

In some aspects, the present disclosure provides a method of preparing a compound, comprising one or more steps as described herein.

In some aspects, the present disclosure provides a compound obtainable by, or obtained by, or directly obtained by a method for preparing a compound described herein.

In some aspects, the present disclosure provides an intermediate being suitable for use in a method for preparing a compound described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl (Bn). The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of the present disclosure has been synthesized by any one of the processes defined herein, the processes may then further comprise the additional steps of: (i) removing any protecting groups present; (ii) converting the compound of the present disclosure into another compound of the present disclosure; (iii) forming a pharmaceutically acceptable salt, hydrate or solvate thereof; and/or (iv) forming a prodrug thereof.

The resultant compounds of the present disclosure can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons). The compounds of the disclosure can be synthesized by the methods described in Schemes 1-7 below. The synthesis of various hydroxyl-substituted heterocycles is well documented in the literature and can be synthesized by known literature methods. The depicted intermediates may also be available as commercial reagents from numerous vendors.

The compounds of the present disclosure can be prepared according to the following general procedures illustrated in the following Schemes. It will be appreciated that, although the general methods may depict the synthesis of certain compounds of the present disclosure, the following general methods, and other methods known to one of ordinary skill in the art, can be applied to all compounds and subclasses and species of each of these compounds, as described herein. The variables, where used in the following General Schemes, can be replaced to match the variable or moieties of any of the Formulae and Compounds disclosed herein as appropriate.

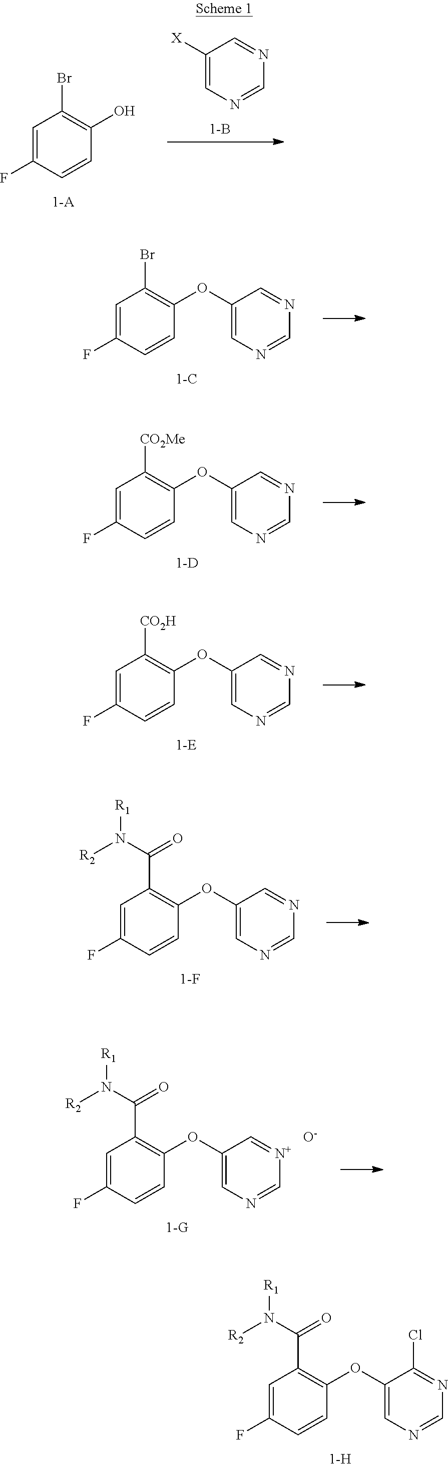

Scheme 1 illustrates the general method for the preparation of key intermediates like compound 1-H. A suitably substituted 2-halophenol, for instance, 4-fluoro-2-bromophenol (1-A), is reacted with a 5-halopyrimidine (1-B), for example 5-bromopyrimidine or 5-iodopyrimidine, to afford the corresponding diaryl ether (1-C). This reaction is typically conducted in the presence of an acid scavenger like Na₂CO₃, K₂CO₃, Cs₂CO₃, K₃PO₄, Et₃N (triethylamine) or (iPr)₂NEt (diispropylethylamine, also known as Hunig's base), in a suitable, neutral solvent, such as DMA (dimethyl acetamide) or NMP (N-methylpyrrolidinone). In some instances, this transformation can be facilitated by addition of an appropriate catalyst, typically Cu₂O or CuI, in the presence of a suitable ligand, for example rel-(1R,2R)—N¹,N²-bis(2-pyridinylmethylene)-1,2-cyclohexanediamine or 3,4,7,8-tetramethyl-1,10-phenanthroline. Conversion of compound 1-C to compound 1-D can be readily achieved by carbonylation under Pd catalysis, in the presence of an acid scavenger and suitable alcohol (typically used as the solvent in the reaction). Oftentimes, Pd(dppf)Cl₂ (dppf=1,1-bis(diphenylphosphino)ferrocene) is used as the palladium catalyst, but other catalyst systems, such as Pd(Ph₃P)₂Cl₂ (Ph₃P=triphenylphosphine) or Pd(OAc)₂ with Ph₃P, can also be used. Commonly, Et₃N or (i-Pr)₂NEt are used as the acid scavengers, and methanol is used as the solvent. These reactions are most typically run under an atmosphere of CO, but other CO sources, for example oxalic acid, can be used in some instances. The ester group of compound 1-D is subsequently saponified to the corresponding carboxylic acid 1-E. Typically, this transformation is accomplished using LiOH, NaOH or KOH, in an aqueous solvent system, such as methanol/water or THF/water. The carboxylic acid is then converted to an amide (1-F). Many different conditions have been developed to achieve this type of transformation, and will be generally familiar to those of skill in the art. For instance, the carboxylic acid can be reacted with thionyl chloride or oxalyl chloride, which forms the corresponding acid chloride. This reaction can be conducted in a suitable, neutral solvent like CH₂Cl₂ or THF, or if thionyl chloride is used, can be run with thionyl chloride as the reactant and solvent, if desired. The acid chloride is subsequently reacted with a suitable amine, R¹R²NH, in the presence of an appropriate acid scavenger, for example Et₃N, (i-Pr)₂NEt, or pyridine, in a neutral solvent like CH₂Cl₂ or THF. In some instances, pyridine can be used as the acid scavenger and the solvent for the reaction.

Sometimes, a strong base like sodium hydride, in a neutral solvent like THF, DMF, or toluene, is used. Alternatively, the carboxylic acid and amine R¹R²NH can be combined together and coupled using a reagent such as DCC (dicyclohexyl carbodiimide), EDC (1-ethyl-3-(4-dimethylaminopropyl)carbodiimide), HATU (1-[bis(dimethylamino)methylene]-1H-1,2,3-triazolo[4,5-b]pyridinium 3-oxide hexafluorophosphate), HBTU (2-(1H-benzotriazol-1-yl)-1,1,3,3-tetramethyluronium hexafluorophosphate), T3P, or POCl₃, in the presence of an appropriate acid scavenger, for example Et₃N, (i-Pr)₂NEt, or pyridine, in a neutral solvent, oftentimes DMF, NMP, CH₂Cl₂ or THF. In some instances, when DCC or EDC is used as the coupling reagent, HOBt (hydroxybenzotriazole) might be added to the reaction to facilitate the desired coupling reaction. Reaction of 1-F with a suitable oxidizing agent, such as urea hydroperoxide in the presence of trifluoroacetic anhydride, or meta-chloroperoxybenzoic acid (mCPBA), in a suitable solvent, typically CH₂Cl₂ or THF, affords the pyrimidine N-oxide 1-G.

This then can be chlorinated to afford compound 1-H using an appropriate chlorinating agent, typically POCl₃ or oxalyl chloride, in the presence of an acid scavenger, usually Et₃N, (i-Pr)₂NEt, in a neutral solvent like CH₂Cl₂, ethyl acetate (EtOAc), or isopropyl acetate.

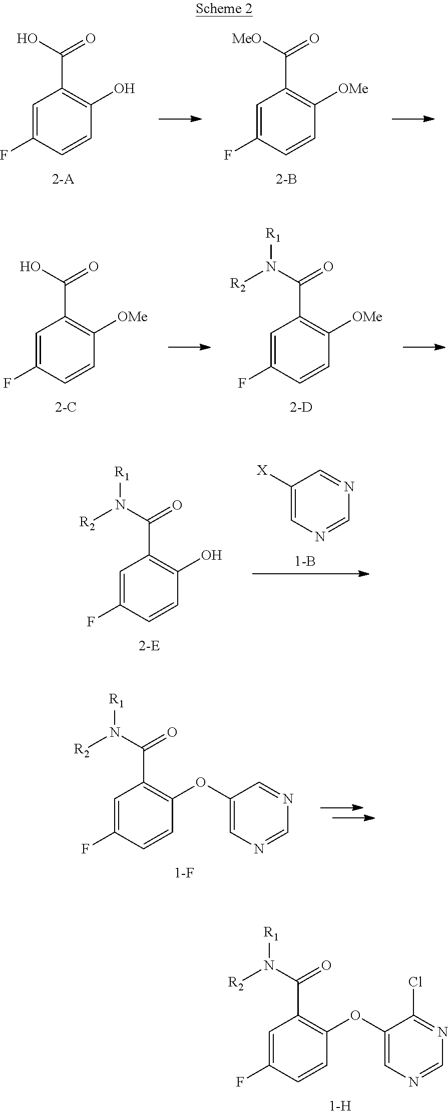

Scheme 2 illustrates an alternative method to prepare the key intermediate 1-H. A suitable salicylic acid derivative, for instance 5-fluoro-2-hydroxybenzoic acid (2-A), is reacted with an alkyl halide, for example, methyl iodide, to afford the corresponding ester/ether derivative 2-B. This reaction is typically conducted in the presence of an acid scavenger, for example K₂CO₃, in a neutral solvent, typically acetone or 2-butanone. The ester group of compound 2-B is subsequently saponified to the corresponding carboxylic acid 2-C, as generally described in Scheme 1, and carboxylic acid 2-C is then converted to amide 2-D, as generally described in Scheme 1. In the present case, a preferred method is to convert carboxylic acid 2-C to the corresponding acid chloride (not shown in the scheme) using SOCl₂ in dichloromethane as solvent. The acid chloride is subsequently reacted with a suitable amine, for example N-ethylpropan-2-amine, in the presence of an acid scavenger, typically Et₃N or (i-Pr)₂NEt, to afford benzamide 2-D. Compound 2-D is then deprotected to afford 2-E. Many different conditions have been developed to achieve this type of transformation and will be generally familiar to those of skill in the art. For instance, reaction of the methyl ether with a strong acid like HBr or HCl, at elevated temperature, affords the corresponding phenol. Alternatively, this transformation can be achieved using a Lewis acid like BBr₃ or AlCl₃, or with a thiolate nucleophile like ethanethiol. The phenol of 2-E is then reacted with a 5-halopyrimidine (1-B), such as 5-bromopyrimidine or 5-iodopyrimidine, to afford the corresponding diaryl ether 1-F, as described in Scheme 1 (1-A to 1-C). The conversion of 1-F to 1-H is accomplished in two steps, in the same manner as described in Scheme 1 (see 1-F to 1-H).

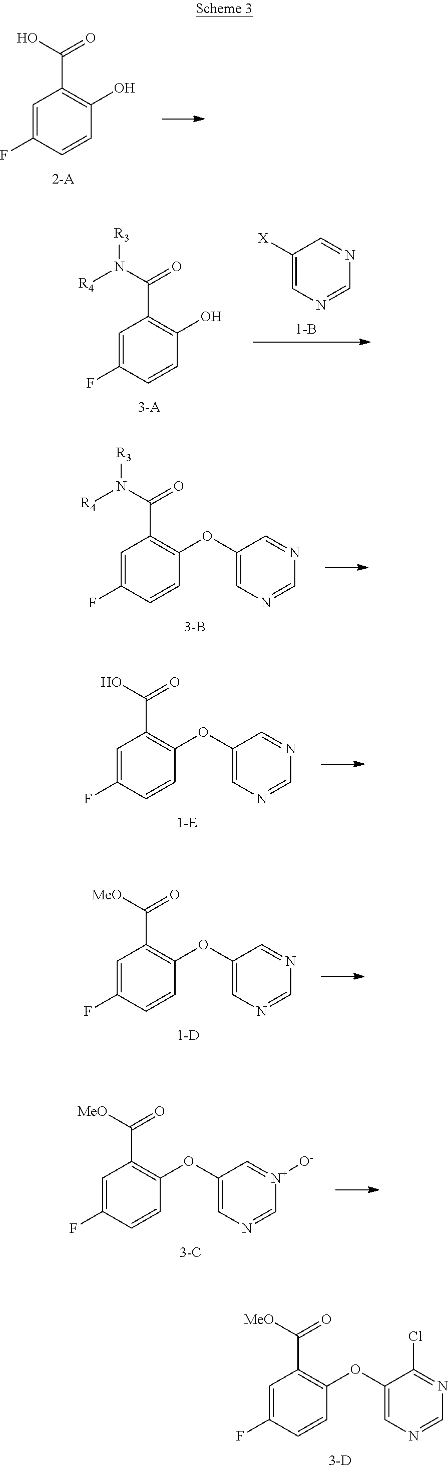

Scheme 3 illustrates the general methods for preparing the key intermediate 3-D. A suitable salicylic acid derivative, for instance 5-fluoro-2-hydroxybenzoic acid (2-A), is converted initially to the amide 3-A, which can be achieved as generally described in Scheme 1 (see 1E to 1F) and Scheme 2 (see 2-C to 2-D). Amide 3-A is then reacted with a 5-halopyrimidine (1-B), such as 5-bromopyrimidine or 5-iodopyrimidine, to afford the corresponding diaryl ether 3-B, as generally described in Scheme 1 (see 1-A to 1-C). Saponification of the amide moiety of 3-B can be achieved by reaction with LiOH, NaOH or KOH, in an aqueous solvent system, such as methanol/water or THF/water. The resulting carboxylic acid is then converted to ester 1-D. This transformation is usually achieved by reacting the carboxylic acid with an alcohol, such as methanol or ethanol, in the presence of an acid catalyst like HCl or H₂SO₄. Typically, the alcohol is used as the solvent, and heat can be used to increase the reaction rate. Alternatively, 1-E can be O-alkylated using methyl iodide in the presence of an acid scavenger, oftentimes K₂CO₃, to afford 1-D. Conversion of 1-D to 3-D is achieved as described in Scheme 1 (see 1-F to 1-H).

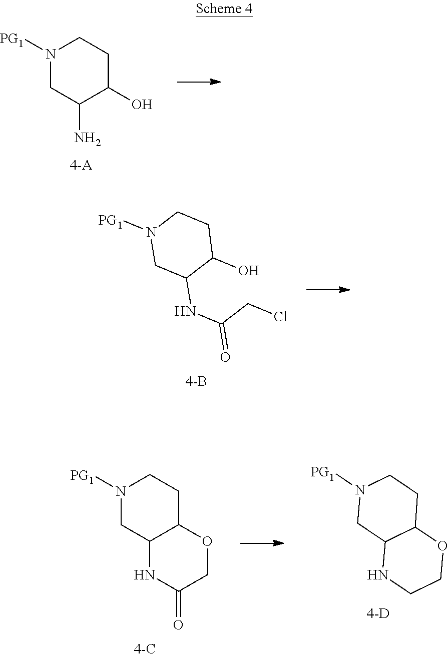

Scheme 4 illustrates the general method for the preparation of key intermediates like compound 4-D. A suitably protected 1,2-amino hydroxy piperidine (for example, tert-butyl (3S,4S)-3-amino-4-hydroxypiperidine-1-carboxylate) is reacted with 2-chloroacetyl chloride to afford the corresponding chloroacetamide 4-B. This reaction is typically conducted in the presence of an acid scavenger like NaHCO₃, Na₂CO₃, K₂CO₃, Cs₂CO₃, K₃PO₄, Et₃N (triethylamine) or (iPr)₂NEt (diisopropylethylamine, also known as Hunig's base), in a suitable, neutral solvent, such as DCM (dichloromethane) or DCE (1,2-dichloroethane). In some instances, strong bases like NaOH or KOH might be used. The subsequent cyclization of 4-B to afford 4-C is also typically achieved under basic conditions. This reaction generally requires a strong base, oftentimes KOtBu, in a solvent like t-BuOH or THF. In some cases, elevated temperature can help to increase the rate of the reaction. Those of skill in the art can envision that the transformation of 4-A directly to 4-C might be achieved in one pot under optimized basic conditions. The amide group of 4-C can be subsequently reduced to the corresponding amine compound 4-D. Many different conditions have been developed to achieve this type of transformation and will be generally familiar to those of skill in the art. Typically, this transformation is accomplished using stoichiometric reductants such as aluminum or boron hydrides (LiAlH₄, BH₃·Et₂O, BH₃·DMS, or 9-BBN) in solvents such as THF, Et₂O, DCM, hexane or toluene. For instance, the amide group in 4-C can be reduced using BH₃·THF in THF solvent to afford amine compound 4-D.

Alternatively, the amide can be reduced to the corresponding amine by initial amide activation using, for instance, Tf₂O (trifluoromethanesulfonic anhydride) in the presence of an acid scavenger like 2-fluoropyridine, or a Lewis acid, such as BF3·Et₂O, followed by reduction with a reducing agent like NaBH₄ or tetramethyldisiloxane.

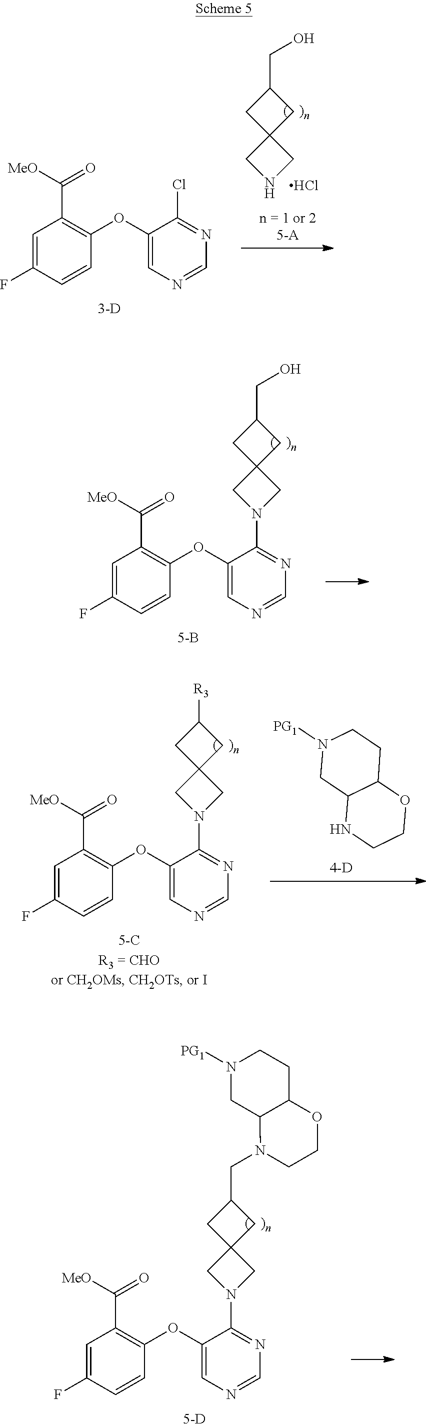

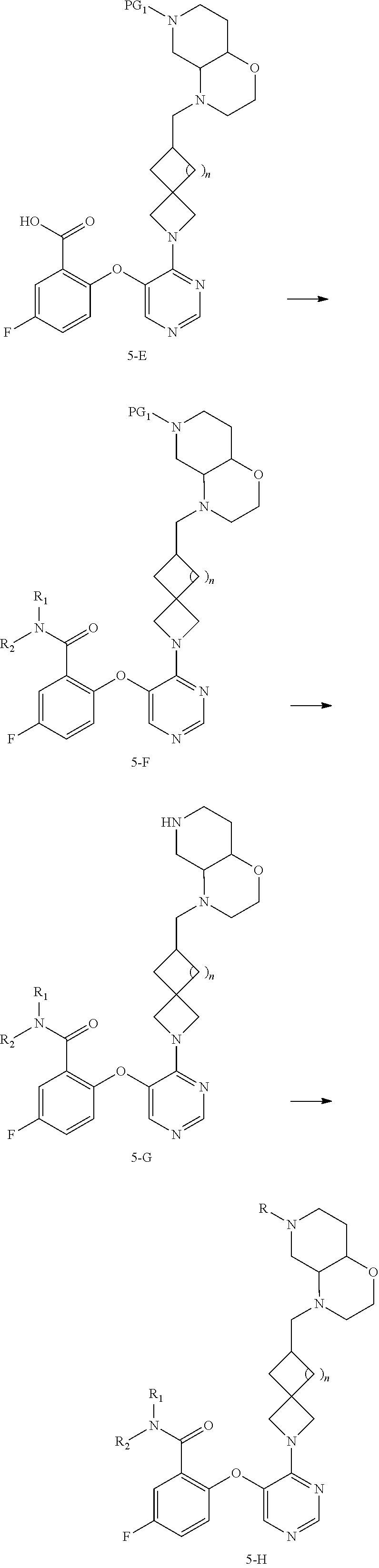

Scheme 5 illustrates the general methods for the preparation of the compounds of the present invention like 5-G and 5-H. Spiro amine compound 5-A reacts with the chloride of compound 3-D to afford compound 5-B. This reaction is conducted in the presence of an acid scavenger, usually Et₃N or (i-Pr)₂NEt, in a suitable, neutral solvent like IPA, n-BuOH, tert-BuOH, tert-amyl alcohol, CH₂Cl₂, THF, DMF, or isopropyl acetate. Subsequently, the alcohol group of 5-B can be converted to the corresponding aldehyde 5-C(R₃=CHO). Those of skill in the art will recognize that many reagents are known for the oxidation of an alcohol to an aldehyde, including PCC (pyridinium chlorochromate), PDC (pyridinium dichromate), DMP (Dess Martin periodinane), IBX (2-iodoxybenzoic acid), and TPAP (tetrapropylammonium perruthenate, also known as the Ley-Griffith reagent). Alternatively, this type of oxidation might be accomplished using TEMPO in combination with NaOCl, or via other well-known reactions like the Swern oxidation and Corey-Kim oxidation. The aldehyde 5-C(R₃=CHO) can be reacted with a suitable amine, for instance compound 4-D, in a reductive amination reaction to afford compound 5-D.

This reaction proceeds via the formation of an intermediate imine or iminium ion (not shown), which is reduced in situ using an appropriate reducing agent. Preferred reducing agents for a reductive amination reaction include NaBH₄ (sodium borohydride), STAB (sodium triacetoxyborohydride), and NaBH₃CN (sodium cyanoborohydride). The reaction is conducted in a suitable, neutral solvent like MeOH, DCE, CH₂Cl₂, THF, DMF, NMP, or a combination thereof. Oftentimes, an acid, usually AcOH (acetic acid), is included to facilitate the reaction. In a representative example, compound 5-C(R₃=CHO) is reacted with amine 4-D (for example, tert-butyl (4aS,8aS)-hexahydro-2H-pyrido[4,3-b][1,4]oxazine-6(5H)-carboxylate) in the presence of NaBH₃CN and AcOH in MeOH to afford compound 5-D. An alternative way to prepare compound 5-D is via an alkylation reaction. In this type of reaction, the alcohol of compound 5-B is first activated by conversion to a leaving group, for instance an alkyl or aryl sulfonate, or a halide. To form an alkyl or aryl sulfonate, the alcohol is reacted with, for example, MsCl (methanesulfonyl chloride) or TsCl (4-toluenesulfonyl chloride) in the presence of acid scavengers like Et₃N or (i-Pr)₂NEt, in a neutral solvent like DCM or DCE. If desired, the sulfonate can be converted to a halide via reaction with an alkali metal halide, for instance potassium iodide (to give the compound where R₃=I). Nucleophilic displacement of the leaving group in 5-C(R₃=CH₂OMs, CH₂OTs, or CH₂I) with amine 4-D affords compound 5-D. This reaction can be conducted in the presence of a base like Na₂CO₃, K₂CO₃, Cs₂CO₃, Et₃N (triethylamine), or (iPr)₂NEt (diispropylethylamine, also known as Hunig's base), in a suitable, neutral solvent, such as ACN, THF, DMF, DMA (dimethyl acetamide) or NMP (N-methylpyrrolidinone).

Saponification of the ester of 5-D to the corresponding carboxylic acid 5-E is accomplished using LiOH, NaOH or KOH, in an aqueous solvent system, such as methanol/water or THF/water.

If desired, the carboxylic acid can also be isolated as the Li, Na, or K salt. The carboxylic acid is then converted to the amide 5-F. Many different conditions have been developed to achieve this type of transformation and will be generally familiar to those of skill in the art. For instance, the carboxylic acid can be reacted with thionyl chloride or oxalyl chloride, which forms the corresponding acid chloride. This reaction can be conducted in a suitable, neutral solvent like CH₂Cl₂, or if thionyl chloride is used, can be run with thionyl chloride as the reactant and solvent. The acid chloride is subsequently reacted with a suitable amine, R1R2NH, in the presence of an appropriate acid scavenger, for example, Et₃N, (i-Pr)₂NEt, or pyridine, in a neutral solvent like CH₂Cl₂ or THF. In some instances, pyridine can be used as the acid scavenger and the solvent for the reaction. Alternatively, the carboxylic acid and amine R1R2NH can be combined and coupled using a reagent such as DCC (dicyclohexyl carbodiimide), EDC (1-ethyl-3-(4-dimethylaminopropyl)carbodiimide), HATU (1-[bis(dimethylamino)methylene]-1H-1,2,3-triazolo[4,5-b]pyridinium 3-oxide hexafluorophosphate), HBTU (2-(1H-benzotriazol-1-yl)-1,1,3,3-tetramethyluronium hexafluorophosphate), or T3P, in the presence of an appropriate acid scavenger, for example, Et₃N, (i-Pr)₂NEt, or pyridine, in a neutral solvent, oftentimes DMF, NMP, CH₂Cl₂ or THF. In some instances, when DCC or EDC is used as the coupling reagent, HOBt (hydroxybenzotriazole) might be added to the reaction to facilitate the desired coupling reaction.

Removal of the protecting group of compound 5-F affords compound 5-G. The use of protecting groups in organic synthesis is well-known to those of skill in the art, and conditions for adding and removing protecting groups are described in established reference volumes, for instance, Greene's Protective Groups in Organic Synthesis, 4^{th} Edition (ISBN:9780470053485). For instance, when the protecting group PG₁ in 5-F is Cbz, deprotection is oftentimes achieved under hydrogenolysis conditions. Hydrogenolysis is accomplished by reaction with hydrogen gas, generally in the presence of a palladium catalyst, such as palladium on activated carbon (Pd/C) or Pd(OH)₂. Sometimes, AcOH or HCl is added to the reaction mixture, and a suitable solvent, like methanol, ethanol, 2,2,2-trifluoroethanol, ethyl acetate, or a combination thereof, is typically used. Sometimes, PtO₂ can be used as a hydrogenolysis catalyst. When the protecting group PG₁ in 5-F is Boc (tert-butyloxycarbonyl), deprotection is oftentimes accomplished under acidic conditions, using acids like HCl, TFA (trifluoracetic acid), or p-TsOH (para-toluenesulfonic acid), in a suitable solvent, oftentimes CH₂Cl₂, THF or 1,4-dioxane. Preferably, the Boc group in compound 5-F is removed with TMSCI in 2,2,2-trifluoroethanol. If desired, 5-G can be N-alkylated to afford compound 5-H (R=an appropriate alkyl group). In this type of reaction, 5-G reacts with an alkyl halide, alkyl triflate, or alkyl tosylate to afford the alkylated product 5-H. This reaction can be conducted in the presence of a base like Na₂CO₃, K₂CO₃, Cs₂CO₃, Et₃N (triethylamine) or (iPr)₂NEt (diispropylethylamine, also known as Hunig's base), in a suitable, neutral solvent, such as ACN, THF, DMF, DMA (dimethyl acetamide) or NMP (N-methylpyrrolidinone). Alternatively, 5-G can be reacted with a suitable aldehyde or ketone in a reductive amination reaction as described previously (see 5-C to 5-D). In a representative example, formaldehyde reacts with 5-G in the presence of STAB (sodium triacetoxyborohydride) and AcOH, in DCE to afford the N-methylated compound 5-H.

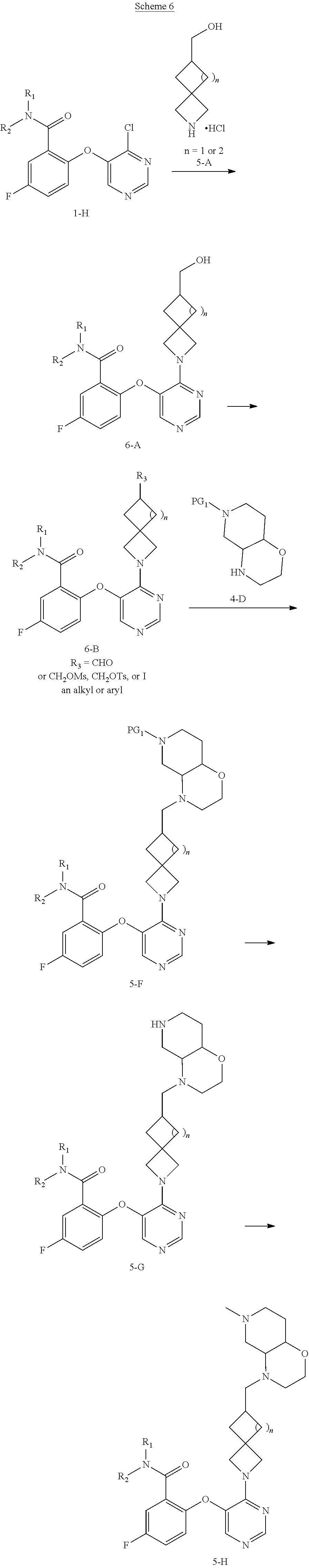

A slightly modified way to prepare the compounds of the present invention like 5-G and 5-H is shown in Scheme 6. In this approach, instead of starting with an ester, as was done in Scheme 5, the sequence begins with the benzamide already in place (see 1-H). Subsequently, according to the general methods described in Scheme 5, compound 1-H, with the benzamide already in place, can be converted to compound 5-F. This compound is then deprotected according to the methods described in Scheme 5 to afford 5-G, which can then be converted to compound 5-H as described in Scheme 5.

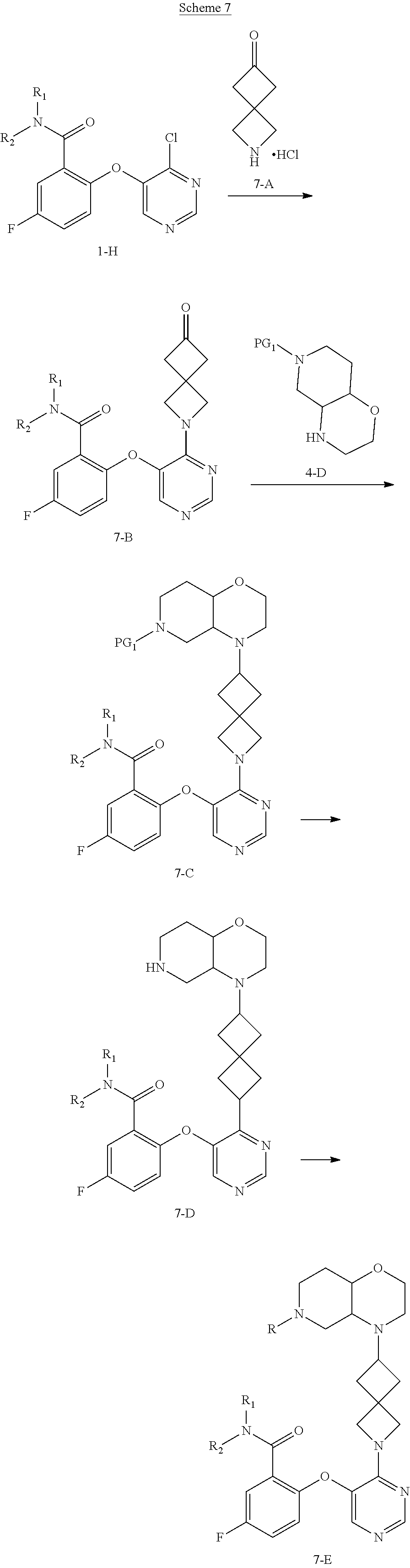

Scheme 7 illustrates the general methods for the preparation of the compounds of the present invention like 7-D and 7-E. Displacement of the aryl chloride of compound 1-H with amine 7-A, according to the general methods for this type of reaction described in Schemes 5 (see 3-D to 5-B) and 6 (see 1-H to 6-A), affords compound 7-B. The ketone of 7-B is then reacted with a suitable amine, for instance 4-D, according to the general conditions for reductive amination described in detail in Scheme 5. Removal of the amine protecting group (PG₁) to afford 7-D, and subsequent N-alkylation to deliver 7-E, is accomplished according to the general methods described in detail in Scheme 5 (see the conversion of 5-F to 5-G, and 5-G to 5-H).

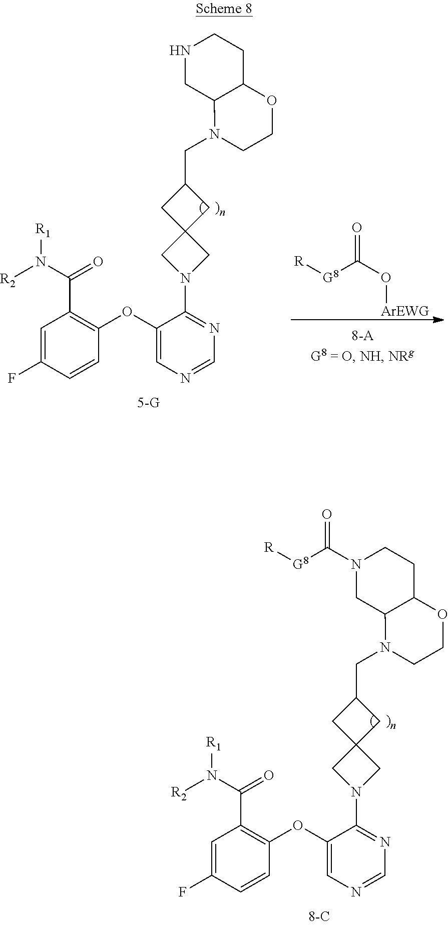

Scheme 8 illustrates additional general methods for the preparation of compounds of the present invention like 8-C. Intermediate 8-A, features an electron-deficient phenyl group (ArEWG) (e.g., nitrophenyl, dinitrophenyl, pentafluorophenyl). Reagent 8-A (wherein G⁸ may be, for example, O, NH, NR^{g}, and wherein R and R^{g} may be any compatible substituent) can be obtained by activation of a compound bearing a nucleophilic residue, such as an amine, alcohol or carboxylic acid, with an appropriate carbonyl source such as bis(4-nitrophenyl) carbonate, bis(2,4-dinitrofluorophenyl) carbonate, or bis(pentafluorophenyl) carbonate. This activation is typically performed in solvents like DCM, THF, Et₂O, or PhMe, with an acid scavenger such as NEt₃ or iPrNEt₂, at reduced or ambient temperatures. The resulting 8-A can then be reacted with 5-G in a suitable solvent, for example ACN, DCM, Et₂O, or THF, in the presence of an appropriate acid scavenger, for example NEt₃ or iPrNEt₂, often at reduced or ambient temperature, to produce the compound 8-C.

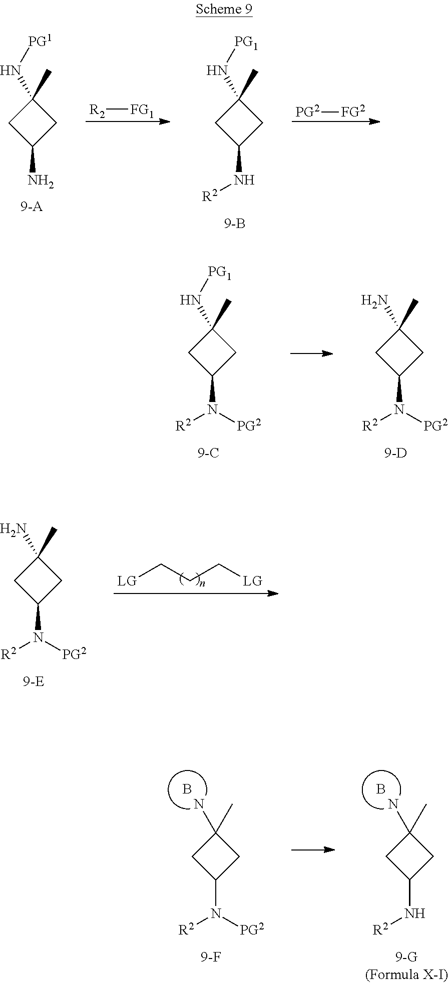

Scheme 9 illustrates a general method to prepare intermediate 9-G (Formula X-I). Protected diamine 9-A, wherein PG¹ can be an appropriate amine protecting group, such as Boc, is reacted with a substituent R² bearing an appropriate functional handle FG₁ (e.g. aldehyde, ketone, or halide), to produce substituted amine 9-B. A second amine protecting group can be installed (e.g. Cbz, wherein PG²-FG² is for example Cbz-Cl) to produce protected diamine 9-C. The first protecting group PG¹ can be selectively removed when for example, PG¹ is Boc, in the presence of TMSCI in a suitable solvent, such as 2,2,2-trifluoroethanol, often at reduced or ambient temperature to afford intermediate 9-E, which can be further functionalized to produce a ring by reaction with a difunctionalized alkyl group (e.g. LG=halide, and n is 0, 1, 2, 3, or 4) to produce 9-F. The second protecting group (e.g. Cbz) can be removed by hydrogenation in the presence of hydrogen gas and a palladium catalyst (e.g. palladium⁰ on activated carbon), in a suitable solvent, such as 2,2,2-trifluroroethanol, often at ambient temperature to produce intermediate 9-G (Formula X-I).

### General Biological Methods

### Menin-MLL Competition and MV4;11 Cell Proliferation Assays

Menin-MLL is a competition assay between human Menin and N-terminal portion of human MLL representing amino acids 4-43 of the protein. The interaction between Menin and MLL peptide was monitored by HTRF employing Terbium labeled anti-His6 antibody directed to the N-terminal His6-tag on recombinant Menin and FITC group covalently attached to the MLL peptide. The N-terminal fragment of MLL, retained in all MLL fusion proteins, is involved in the interactions with Menin, and this protein-protein interaction is critical for the MLL fusion proteins mediated leukemogenic transformations.

For IC₅₀ determination test compounds may be prepared as stock solutions. Lower sub-stocks of 50 μM may be prepared from the 10 mM stock solution. To test the compounds in assay, 3.16-fold serial dilutions are made in 100% DMSO. Mid-stock of 50× compounds (50 μM) were serially diluted (3.16 fold) in 100% DMSO in Polypropylene plate. In assay plate 1 micro-litre of the previously prepared compound dilution was stamped. H-FL-Menin diluted to 4 nM in assay buffer (50 mM Tris-HCl, pH 7.4, 50 mM NaCl, freshly prepared 1 mM DTT, 0.01% BSA, 0.005% Triton X-100) was pre-incubated with 8 nM anti-His6-Tb for 30 min at room temperature. FITC-MLL-4-43 was diluted to 2 nM in assay buffer and 25 μL was dispensed into each well of the assay plate followed by addition of 25 μl of pre-incubated H-FL-Menin and anti-His6-Tb mixture. Final concentration H-FL-Menin diluted to 1 nM in assay plate with 2 nM anti-His6-Tb and 1 nM FITC-MLL-4-43. After 1 hr incubation at room temperature, the HTRF signal was measured on the Spark multi-label plate reader. Resulting data were captured as a ratio of RFU520/RFU485×1000. The max values were obtained from 0% inhibition in presence of 2% DMSO and the min. values were 100% inhibition in presence of 1 M reference compound.

### Cell Proliferation Assay—MV4;11

Compounds were evaluated for its capacity to inhibit the proliferation of the MLLr leukemia cell line MV4-11 that harbors an MLL1-AF4 fusion protein. MV4-11 cells were cultured for 72 hours with limiting dilutions of compounds and viability was measured using CellTitert-Glo.

Compounds were dissolved to obtain as 10 mM solution in DMSO. The stock was diluted 1:5 to the top concentration of 2 mM in 100% DMSO. For IC₅₀ determination, serial 1:3.16 dilutions were prepared in 100% DMSO by diluting 20 μL into 43.5 μL of DMSO for 8 concentrations. Each prepared DMSO solution were further diluted 1:500 in the cell culture media to obtain the 2× dosing solutions. The final concentrations of tested compounds in the cell culture media ranged from 0.632 nM to 2000 nM.

MV4-11 cells were cultured in IMDM with 10% FBS and 1× Penicillin-Streptomycin at 5% CO₂ and 37° C. A cell suspension was prepared containing 15,000 cells/ml in the culture medium and 100 μL of this suspension was added per well to a 96-well cell culture plate. Then, 100 μL of 2× dosing media containing test compounds were added bringing the total volume to 200 μL. These cells were cultured for 72 hours at 37° C. and 5% CO₂ in a humidified incubator.

After 72 hours, the cultured cells were mixed and 100 μL was transferred to a 96-well black plate. Cell Titer Glo (100 μl) was then added to this plate. The plate was mixed with shaking for 15 mins at RT, the luminescence was then measured using Tecan Spark 20M spectrophotometer. Cell Viability (%) was determined by RLU of test/RLU average vehicle control*100 and % max inhibition was determined by 100−(% cell viability remaining at the top concentration of compound).

hERG Patch Clamp Assay

The hERG inhibition assay uses a high throughput single cell planar patch clamp approach. Chinese hamster ovary cells transfected with the hERG gene (CHO-hERG) are dispensed into the PatchPlate. Amphotericin is used as a perforating agent to gain electrical access to the cells. The hERG tail current is measured prior to the addition of the test compound by perforated patch clamping. Following addition of the test compound at a defined concentration or range of concentrations a second recording of the hERG current is performed. The degree of inhibition (%) is obtained by measuring the tail current amplitude, which is induced by a one second test pulse to −40 mV after a two second pulse to +20 mV, before and after drug incubation (the difference current is normalized to control and multiplied by 100 to obtain the percent of inhibition). The patch clamp assay can be used according to the knowledge of a person of ordinary skill in the art to accordingly assess the compounds of the present disclosure.

In any one of the embodiments described herein, the compound has an IC₅₀ of more than 10, 15, 20, 25, or 30 μM in a standard human ether-a-go-go related gene (hERG) patch clamp assay.

A number of drugs have been withdrawn from late-stage clinical trials due to cardiotoxic effects, therefore it is important to identify and avoid compounds with potential for cardiotoxic effects early in drug discovery. The cardiovascular toxicity of a compound can be measured using a standard human ether-a-go-go related gene (hERG) assay. The human ether-a-go-go related gene (hERG) encodes the inward rectifying voltage gated potassium channel in the heart (IKr), which is involved in cardiac repolarization. Inhibition of the hERG current causes QT interval prolongation resulting in potentially fatal ventricular tachyarrhythmia called Torsade de Pointes. A compound having an IC₅₀ of more than about 10 μM or more than about 15 μM, in the hERG assay may be considered as free from any cardiovascular toxicity. In some embodiments, the compounds of Formulae I, II, II-a, II-b, II-c, II-d, and II-e, and/or Table 1 have reduced hERG binding compared to structural analogs. In some embodiments, the compounds of Formulae I, II, II-a, II-b, II-c, II-d, and II-e, and/or Table 1 have IC₅₀ of more than 10 μM, 15 μM, 20 μM, 25 μM, or 30 μM in the standard patch clamp hERG assay.

In some embodiments, the compounds of the present disclosure (e.g., Formulae I, II, II-a, II-b, II-c, II-d, and II-e, and Table 1) do not significantly block the hERG potassium channel (e.g., an IC₅₀ greater than 1 μM, 5 μM, 10 μM, 15 μM, 20 μM, 25 μM, 30 μM, 35 μM, 40 μM, or 50 μM) in the standard patch clamp hERG assay.

### Methods of Use

The compounds of the invention are inhibitors of the interaction of menin with MLL and MLL fusion proteins. In some embodiments, the present disclosure is directed to a method of inhibiting the interaction between menin and MLL or an MLL fusion protein by contacting menin and MLL or the MLL fusion protein with a compound of the disclosure. The contacting can be carried out in vitro or in vivo. In some embodiments, the compounds of the disclosure can bind to menin, thereby interfering with the binding of MLL to menin. In some embodiments, the present disclosure provides a method of inhibiting the activity of menin by contacting menin with a compound of the disclosure in the presence of MLL or an MLL fusion protein. In further embodiments, the present disclosure provides a method of inhibiting the binding of MLL or an MLL fusion protein to menin, comprising contacting menin with a compound of the disclosure in the presence of the MLL or MLL fusion protein.

In some embodiments, compounds of the present disclosure minimize hERG interactions. In some embodiments, the present disclosure is directed to a method of inhibiting the interaction between menin and MLL or an MLL fusion protein by contacting menin and MLL or the MLL fusion protein with a compound of the disclosure while the compounds of the disclosure minimize hERG activity. In some embodiments, the present disclosure is directed to a method of inhibiting the interaction between menin and MLL or an MLL fusion protein by contacting menin and MLL or the MLL fusion protein with a compound of the disclosure while the compound of the disclosure avoids drug-induced blockade of hERG.

Evaluating the hERG activity can be accomplished by many methods known in the art. Including, such methods for the assessment of hERG liability is the patch-clamp electrophysiological assay on hERG transfected cells. Various other strategies including radiolabeled binding assays, functional assays, and rubidium efflux assays also quantify hERG potency.

The compounds of the disclosure are also useful in treating diseases associated with the menin-HLL interaction or menin-MLL fusion protein interaction. For example, diseases and conditions treatable according to the methods of the disclosure include cancer, such as leukemia, and other diseases or disorders mediated by the menin-MLL interaction or menin-MLL fusion protein interaction such as diabetes.

Accordingly, the compounds of the disclosure are believed to be effective against a broad range of cancers, including, but not limited to, hematological cancer (e.g., leukemia and lymphoma), bladder cancer, brain cancer (e.g., glioma, diffuse intrinsic pontine glioma (DIPG)), breast cancer (e.g., triple-negative breast cancer, estrogen-receptor-positive breast cancer (i.e., ER+ breast cancer)), colorectal cancer, cervical cancer, gastrointestinal cancer (e.g., colorectal carcinoma, gastric cancer), genitourinary cancer, head and neck cancer, liver cancer, lung cancer, melanoma, ovarian cancer, pancreatic cancer, prostate cancer (e.g., castration resistant prostate cancer), renal cancer (e.g., renal cell carcinoma), skin cancer, thyroid cancer (e.g., papillary thyroid carcinoma), testicular cancer, sarcoma (e.g., Ewing's sarcoma), and AIDS-related cancers. In some embodiments, the cancer is associated with a rearranged MLL gene. In some embodiments, the pathophysiology of the cancer is dependent on the MLL gene. In some embodiments, the cancer is associated with mutant p53 gain-of-function.

In some embodiments, the specific cancers that may be treated by the compounds, compositions and methods described herein include cardiac cancers, such as for example, sarcoma (e.g., angiosarcoma, fibrosarcoma, rhabdomyosarcoma, and liposarcoma), myxoma, rhabdomyoma, fibroma, lipoma and teratoma; lung cancers, including, for example, bronchogenic carcinoma (e.g., squamous cell, undifferentiated small cell, undifferentiated large cell, and adenocarcinoma), alveolar and bronchiolar carcinoma, bronchial adenoma, sarcoma, lymphoma, chondromatous hamartoma, mesothelioma, non-small cell lung cancer, small cell lung cancer, bronchial adenomas/carcinoids, and pleuropulmonary blastoma; gastrointestinal cancer, including, for example, cancers of the esophagus (e.g., squamous cell carcinoma, adenocarcinoma, leiomyosarcoma, and lymphoma), cancers of the stomach (e.g., carcinoma, lymphoma, and leiomyosarcoma), cancers of the pancreas (e.g., ductal adenocarcinoma, insulinoma, glucagonoma, gastrinoma, carcinoid tumors, and vipoma), cancers of the small bowel (e.g., adenocarcinoma, lymphoma, carcinoid tumors, Kaposi's sarcoma, leiomyoma, hemangioma, lipoma, neurofibroma, and fibroma), cancers of the large bowel or colon, (e.g., adenocarcinoma, tubular adenoma, villous adenoma, hamartoma, and leiomyoma), and other cancers of the digestive tract (e.g., anal cancer, anorectal cancer, appendix cancer, cancer of the anal canal, cancer of the tongue, gallbladder cancer, gastrointestinal stromal tumor (GIST), colon cancer, colorectal cancer, extrahepatic bile duct cancer, intrahepatic bile duct cancer, rectal cancer, and small intestine cancer); genitourinary tract cancers, including, for example, cancers of the kidney (e.g., adenocarcinoma, Wilm's tumor (nephroblastoma), lymphoma, and leukemia), cancers of the bladder and urethra (e.g., squamous cell carcinoma, transitional cell carcinoma, and adenocarcinoma), cancers of the prostate (e.g., adenocarcinoma and sarcoma), cancers of the testis, (e.g., seminoma, teratoma, embryonal carcinoma, teratocarcinoma, choriocarcinoma, sarcoma, interstitial cell carcinoma, fibroma, fibroadenoma, adenomatoid tumors, and lipoma), as well as transitional cell cancer, transitional cell cancer of the renal pelvis and ureter and other urinary organs, urethral cancer, and urinary bladder cancer; liver cancers, including, for example, hepatoma (e.g., hepatocellular carcinoma), cholangiocarcinoma, hepatoblastoma, angiosarcoma, hepatocellular adenoma, and hemangioma; bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; nervous system cancers, including, for example, cancers of the skull (e.g., osteoma, hemangioma, granuloma, xanthoma, and osteitis deformans); cancers of the meninges (e.g., meningioma, meningiosarcoma, and gliomatosis); cancers of the brain (e.g., astrocytoma, medulloblastoma, glioma, ependymoma, germinoma (pinealoma), glioblastoma multiforme, oligodendroglioma, schwannoma, retinoblastoma, and congenital tumors); cancers of the spinal cord (e.g., neurofibroma, meningioma, glioma, and sarcoma), and other nervous system cancers (e.g., brain stem glioma, diffuse intrinsic pontine glioma (DIPG), brain tumor, central nervous system cancer, cerebellar astrocytoma, cerebral astrocytoma/malignant glioma, childhood cerebellar astrocytoma, childhood cerebral astrocytoma, primary central nervous system lymphoma, visual pathway and hypothalamic glioma, nervous system lymphoma, supratentorial primitive neuroectodeimal tumors, pineoblastoma and supratentorial primitive neuroectodermal tumors); gynecological cancers, including, for example, cancers of the uterus (e.g., endometrial carcinoma), cancers of the cervix (e.g., cervical carcinoma, and pre tumor cervical dysplasia), cancers of the ovaries (e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma), cancers of the vulva (e.g., squamous cell carcinoma, intraepithelial carcinoma, adenocarcinoma, fibrosarcoma, and melanoma), cancers of the vagina (e.g., clear cell carcinoma, squamous cell carcinoma, botryoid sarcoma, and embryonal rhabdomyosarcoma), and cancers of the fallopian tubes (e.g., carcinoma); other reproductive tract cancers, including, for example, endometrial cancer, endometrial uterine cancer, germ cell tumor, gestational trophoblastic tumor, gestational trophoblastic tumor glioma, ovarian epithelial cancer, ovarian germ cell tumor, ovarian low malignant potential tumor, penile cancer, vaginal cancer, vulvar cancer, extracranial germ cell tumor, extragonadal germ cell tumor, uterine cancer, uterine corpus cancer, uterine sarcoma; lymphatic and hematologic cancers, including, for example, cancers of the blood (e.g., acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenstrom's macroglobulinemia), and other lymphatic or hematologic cancers including, for example, childhood leukemia, myeloproliferative disorders (e.g., primary myelofibrosis), plasma cell neoplasm/multiple myeloma, myelodysplasia, myelodysplastic syndrome, cutaneous T-cell lymphoma, lymphoid neoplasm, AIDS-related lymphoma, thymoma, thymoma and thymic carcinoma, mycosis fungoides, and Sezary Syndrome; skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, merkel cell carcinoma, merkel cell skin carcinoma, melanoma, and carcinoid tumor; adrenal gland cancers, including, for example, neuroblastoma; other cancers associated with the endocrine system including, for example, adrenocortical carcinoma, multiple endocrine neoplasia (e.g., multiple endocrine neoplasia type I), multiple endocrine neoplasia syndrome, parathyroid cancer, pituitary tumor, pheochromocytoma, islet cell pancreatic cancer, and islet cell tumors); connective tissue cancer (e.g., bone cancer, bone and joint cancer, osteosarcoma and malignant fibrous histiocytoma); cancer associated with the head, neck, and mouth (e.g., head and neck cancer, paranasal sinus and nasal cavity cancer, metastatic squamous neck cancer, mouth cancer, throat cancer, esophageal cancer, laryngeal cancer, pharyngeal cancer, hypopharyngeal cancer, lip and oral cavity cancer, nasopharyngeal cancer, oral cancer, oropharyngeal cancer, and salivary gland cancer); and cancer associated with the eye (e.g., ocular cancer, intraocular melanoma). In some embodiments, the cancer is Ewing's sarcoma.

In some embodiments, the cancer is a hematological cancer such as leukemia or lymphoma. Example leukemia and lymphomas treatable by the compounds of the disclosure include mixed lineage leukemia (MLL), MLL-related leukemia, MLL-associated leukemia, MLL-positive leukemia, MLL-induced leukemia, rearranged mixed lineage leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL) (also referred to as acute lymphoblastic leukemia or acute lymphoid leukemia), acute myeloid leukemia (AML) (also referred to as acute myelogenous leukemia or acute myeloblastic leukemia), acute granulocytic leukemia, acute nonlymphocytic leukemia, chronic lymphocytic leukemia (CLL) (also referred to as chronic lymphoblastic leukemia), chronic myelogenous leukemia (CML) (also referred to as chronic myeloid leukemia), therapy related leukemia, myelodysplastic syndrome (MDS), myeloproliferative disease (MPD) (such as primary myelofibrosis (PMF)), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non-Hodgkin's lymphoma (malignant lymphoma), and Waldenstrom's macroglobulinemia. In some embodiments, the acute myeloid leukemia (AML) is abstract nucleophosmin (NPM1)-mutated acute myeloid leukemia (i.e., NPM1^{mut} acute myloid leukemia).

In particular embodiments, compounds of the disclosure are used to treat a rearranged mixed lineage (KMT2A-rearranged) leukemia (MLL-r).

In particular embodiments, compounds of the disclosure are used to treat leukemia associated with a MLL rearrangement, acute lymphocytic leukemia associated with a MLL rearrangement, acute lymphoblastic leukemia associated with a MLL rearrangement, acute lymphoid leukemia associated with a MLL rearrangement, acute myeloid leukemia associated with a MLL rearrangement, acute myelogenous leukemia associated with a MLL rearrangement, or acute myeloblastic leukemia associated with a MLL rearrangement. As used herein, “MLL rearrangement” means a rearrangement of the MLL gene.

In some embodiments, diseases and conditions treatable with compounds of the disclosure include insulin resistance, pre-diabetes, diabetes (e.g., Type 2 diabetes or Type 1 diabetes), and risk of diabetes. In some embodiments, diseases and conditions treatable with compounds of the disclosure include hyperglycemia. In some embodiments, the hyperglycemia is associated with diabetes, such as Type 2 diabetes. In some embodiments, compounds of the disclosure are used to treat loss of response to other anti-diabetic agents and/or reduced beta cell function in a patient or subject. In some embodiments, compounds of the disclosure are used to restore response to other anti-diabetic agents and/or to restore beta cell function and/or to reduce the need for insulin in a patient or subject. In some embodiments, compounds of the disclosure are used to reduce insulin resistance, reduce the risk of diabetes, or reduce increases in blood glucose caused by a statin in a subject taking a statin. In some embodiments, compounds of the disclosure are used to treat diabetes in a subject taking a statin or to prevent diabetes in a subject taking a statin. Methods of the disclosure include decreasing, reducing, inhibiting, suppressing, limiting or controlling in the patient elevated blood glucose levels. In further aspects, methods of the disclosure include increasing, stimulating, enhancing, promoting, inducing or activating in the subject insulin sensitivity. Statins include, but are not limited to atorvastatin, cerivastatin, fluvastatin, lovastatin, mevastatin, pitavastatin, pravastatin, rousuvastatin and simvastatin.

In some embodiments, a patient is treated with (e.g., administered) a compound of the present disclosure in an amount sufficient to treat or ameliorate one or more of the diseases and conditions recited above (e.g., a therapeutically effective amount). The compounds of the disclosure may also be useful in the prevention of one or more of the diseases recited therein.

In some embodiments, a compound of the present disclosure or a pharmaceutically acceptable salt thereof, or a pharmaceutical composition of the present disclosure is used in treating or preventing a disease caused by, or associated with, menin expression, activity, and/or function.

In some embodiments, a compound of the present disclosure or a pharmaceutically acceptable salt thereof, or a pharmaceutical composition of the present disclosure is used in treating or preventing cancer.

In some embodiments, a compound of the present disclosure or a pharmaceutically acceptable salt thereof, or a pharmaceutical composition of the present disclosure is used for treating or preventing a disease caused by, or associated with, menin expression, activity, and/or function.

In some embodiments, a compound of the present disclosure or a pharmaceutically acceptable salt thereof, or a pharmaceutical composition of the present disclosure is used in the manufacture of a medicament for treating or preventing a disease caused by, or associated with, menin expression, activity, and/or function.

In some embodiments, the present disclosure relates to a kit comprising a compound of the present disclosure or a pharmaceutically acceptable salt thereof, or a pharmaceutical composition of the present disclosure and instructions for its use.

In some embodiments, a compound of the present disclosure or a pharmaceutically acceptable salt thereof, or a pharmaceutical composition of the present disclosure is used for treating hematological malignancies. In some embodiments, a compound of the present disclosure or a pharmaceutically acceptable salt thereof, or a pharmaceutical composition of the present disclosure is used for treating hematological malignancies as described in WO2023150635A1, which disclosure is incorporated by reference herein.

In some embodiments, a compound of the present disclosure or a pharmaceutically acceptable salt thereof, or a pharmaceutical composition of the present disclosure is used for treating hematologic cancer with high interferon regulatory factor 8 (IRF8) expression. In some embodiments, a compound of the present disclosure or a pharmaceutically acceptable salt thereof, or a pharmaceutical composition of the present disclosure is used for treating hematologic cancer with high interferon regulatory factor 8 (IRF8) expression as described in WO2025015152A1, which disclosure is incorporated by reference herein.

In some embodiments, a compound of the present disclosure or a pharmaceutically acceptable salt thereof, or a pharmaceutical composition of the present disclosure is used for treating PIK3CA mutation colon cancer. In some embodiments, a compound of the present disclosure or a pharmaceutically acceptable salt thereof, or a pharmaceutical composition of the present disclosure is used for treating PIK3CA mutation colon cancer as described in CN Patent No. 118021815B, which disclosure is incorporated by reference herein.

In some embodiments, a compound of the present disclosure or a pharmaceutically acceptable salt thereof, or a pharmaceutical composition of the present disclosure is used for treating sickle cell disease. In some embodiments, a compound of the present disclosure or a pharmaceutically acceptable salt thereof, or a pharmaceutical composition of the present disclosure is used for treating sickle cell disease as described in U.S. Patent Publication No. 20240100049A1, which disclosure is incorporated by reference herein.

### Combination Therapy

The disclosure further relates to a combination therapy for treating a disease or a disorder described herein. In some embodiments, the combination therapy comprises administering at least one compound of the present disclosure in combination with one or more other pharmaceutically active agents for treating cancer or other disorders mediated by menin/MLL. In some embodiments, the combination therapy comprises administering at least one compound of the present disclosure in combination with one or more other pharmaceutically active agents, such as for the treatment of cancer. The pharmaceutically active agents can be combined with a compound of the disclosure in a single dosage form, or the therapeutics can be administered simultaneously or sequentially as separate dosage forms.

In some embodiments, the invention provides a combination therapy comprising a menin inhibitor of the present disclosure (e.g., a compound of Formula I, Formula II, Formula II-a, Formula II-b, Formula II-c, Formula II-d, Formula II-e, etc.) and a CYP3A4 inhibitor. In certain embodiments, the invention provides for a pharmaceutical composition comprising: (a) a menin inhibitor of the present disclosure (e.g., a compound of Formula I, Formula II, Formula II-a, Formula II-b, Formula II-c, Formula II-d, Formula II-e, etc.), and (b) a CYP3A4 inhibitor. In some embodiments, the invention is directed to a method for treating a patient comprising (a) administering a menin inhibitor of the present disclosure (e.g., a compound of Formula I, Formula II, Formula II-a, Formula II-b, Formula II-c, Formula II-d, Formula II-e, etc.), and (b) administering a CYP3A4 inhibitor.

Some embodiments of this invention are directed to combination therapies designed to treat or manage cancer in a subject, wherein the combination therapies comprise administering a menin inhibitor of the present disclosure (e.g., a compound of Formula I, Formula II, Formula II-a, Formula II-b, Formula II-c, Formula II-d, Formula II-e, etc.) in combination with a CYP3A4 inhibitor. In particular, some embodiments of this invention are directed to methods of treating or managing cancer in a subject, comprising administering a menin inhibitor in combination with a therapeutically effective amount of a CYP3A4 inhibitor administered simultaneously, separately or sequentially.

In some embodiments, the CYP3A inhibitor is: an antiarrhythmic; an antihistamine; an azole antifungal; a benzodiazepine; a calcium channel blocker; a HIV antiviral; a HMG CoA Reductase inhibitor; a macrolide antibiotic; a prokinetic; a protease inhibitor; or any combinations thereof. In some embodiments, the CYP3A inhibitor is: posaconazole, alprazolam; amiodarone; amlodipine; aprepitant; aripiprazole; astemizole; atorvastatin; boceprevir; buspirone; chloramphenicol; chlorpheniramine; cimetidine; ciprofloxacin; cisapride; clarithromycin; cobicistat (GS-9350); analogs or derivatives of cobicistat (GS-9350); cyclosporine; delaviridine; diazepam→3-OH; diethyl-dithiocarbamate; diltiazem; erythromycin; felodipine; fluconazole; fluvoxamine; gestodene; gleevec; grapefruit juice; haloperidol; imatinib; indinavir; itraconazole; ketoconazole; lovastatin; methadone; mibefradil; midazolam; mifepristone; nefazodone; nelfinavir; nifedipine; nisoldipine; nitrendipine; norfloxacin; norfluoxetine; pimozide; quinine; quinidine→3-OH; ritonavir; saquinavir; sildenafil; simvastatin; starfruit; tacrolimus (FK506); tamoxifen; telaprevir; telithromycin; trazodone; triazolam; verapamil; telaprevir; vincristine; voriconazole; or any combinations thereof.

In some embodiments, the CYP3A4 inhibitor is posaconazole, cobicistat (GS-9350) or analogs or derivatives of cobicistat (GS-9350). In some embodiments, the CYP3A4 inhibitor is ketoconazole. In some embodiments, the CYP3A4 inhibitor is ritonavir. In some embodiments, the menin inhibitor and the CYP3A4 inhibitor are in separate dosage forms. In some embodiments, the pharmaceutical composition is in a combined dosage form. In some embodiments, the CYP3A4 inhibitor is posaconazole.

In some embodiments, the pharmaceutical composition comprises an amount of the CYP3A4 inhibitor that is effective to increase the oral bioavailability of the menin inhibitor. The compounds according to the disclosure may also be used in combination with immunotherapies, including but not limited to cell-based therapies, antibody therapies and cytokine therapies, for the treatment of a disease or disorder disclosed herein.

In certain embodiments, compounds according to the disclosure are used in combination with one or more passive immunotherapies, including but not limited to naked monoclonal antibody drugs and conjugated monoclonal antibody drugs. Examples of naked monoclonal antibody drugs that can be used include, but are not limited to, rituximab (Rituxan®), an antibody against the CD20 antigen; trastuzumab (Herceptin®), an antibody against the HER2 protein; alemtuzumab (Lemtrada®, Campath®), an antibody against the CD52 antigen; cetuximab (Erbitux®), an antibody against the EGFR protein; and bevacizumab (Avastin®) which is an anti-angiogenesis inhibitor of VEGF protein.

Examples of conjugated monoclonal antibodies that can be used include, but are not limited to, radiolabeled antibody ibritumomab tiuxetan (Zevalin®); radiolabeled antibody tositumomab (Bexxar®); and immunotoxin gemtuzumab ozogamicin (Mylotarg®) which contains calicheamicin; BL22, an anti-CD22 monoclonal antibody-immunotoxin conjugate; radiolabeled antibodies such as OncoScint® and ProstaScint®; brentuximab vedotin (Adcetris®); ado-trastuzumab emtansine (Kadcyla®, also called TDM-1).

Further examples of therapeutic antibodies that can be used include, but are not limited to, REOPRO® (abciximab), an antibody against the glycoprotein IIb/IIIa receptor on platelets; ZENAPAX® (daclizumab) an immunosuppressive, humanized anti-CD25 monoclonal antibody; PANOREX™, a murine anti-17-IA cell surface antigen IgG2a antibody; BEC2, a murine anti-idiotype (GD3 epitope) IgG antibody; IMC-C225, a chimeric anti-EGFR IgG antibody; VITAXIN™ a humanized anti-αVβ3 integrin antibody; Campath 1H/LDP-03, a humanized anti CD52 IgG1 antibody; Smart M195, a humanized anti-CD33 IgG antibody; LYMPHOCIDE™, a humanized anti-CD22 IgG antibody; LYMPHOCIDE™ Y-90; Lymphoscan; Nuvion® (against CD3; CM3, a humanized anti-ICAM3 antibody; IDEC-114 a primatized anti-CD80 antibody; IDEC-131 a humanized anti-CD40L antibody; IDEC-151 a primatized anti-CD4 antibody; IDEC-152 a primatized anti-CD23 antibody; SMART anti-CD3, a humanized anti-CD3 IgG; 5G1.1, a humanized anti-complement factor 5 (C5) antibody; D2E7, a humanized anti-TNF-α antibody; CDP870, a humanized anti-TNF-α Fab fragment; IDEC-151, a primatized anti-CD4 IgG1 antibody; MDX-CD4, a human anti-CD4 IgG antibody; CD20-streptdavidin (+biotin-yttrium 90); CDP571, a humanized anti-TNF-α IgG4 antibody; LDP-02, a humanized anti-α4β7 antibody; OrthoClone OKT4A, a humanized anti-CD4 IgG antibody; ANTOVA™, a humanized anti-CD40L IgG antibody; ANTEGREN™, a humanized anti-VLA-4 IgG antibody; and CAT-152, a human anti-TGF-β₂ antibody.

In certain embodiments, compounds according to the disclosure are used in combination with one or more targeted immunotherapies containing toxins but not an antibody, including but not limited to denileukin diftitox (Ontak®), IL-2 linked to diphtheria toxin.

The compounds according to the disclosure may also be used in combination with adjuvant immunotherapies for the treatment of a disease or disorder disclosed herein. Such adjuvant immunotherapies include, but are not limited to, cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guerin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of interleukins, for example IL-2, with other cytokines, such as IFN-alpha.

In certain embodiments, compounds according to the disclosure are used in combination with vaccine therapy, including but not limited to autologous and allogeneic tumor cell vaccines, antigen vaccines (including polyvalent antigen vaccines), dendritic cell vaccines, and viral vaccines.

In another embodiment, the present disclosure comprises administering to a subject with cancer an effective amount of a compound of the disclosure and one or more additional anti-cancer therapies selected from: surgery, anti-cancer agents/drugs, biological therapy, radiation therapy, anti-angiogenesis therapy, immunotherapy, adoptive transfer of effector cells, gene therapy or hormonal therapy. Examples of anti-cancer agents/drugs are described below.

In some embodiments, the anti-cancer agents/drug is, for example, adriamycin, aactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; palbociclib; Yervoy® (ipilimumab); Mekinist™ (trametinib); peginterferon alfa-2b, recombinant interferon alfa-2b; Sylatron™ (peginterferon alfa-2b); Tafinlar® (dabrafenib); Zelboraf® (vemurafenib); or nivolumab.

The compounds according to the present disclosure can be administered in combination with existing methods of treating cancers, for example by chemotherapy, irradiation, or surgery. Thus, there is further provided a method of treating cancer comprising administering an effective amount of a compound of the disclosure, or a pharmaceutically acceptable salt form thereof, to a subject in need of such treatment, wherein an effective amount of at least one additional cancer chemotherapeutic agent is administered to the subject. Examples of suitable cancer chemotherapeutic agents include any of: abarelix, ado-trastuzumab emtansine, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, emtansine, epirubicin, eribulin, erlotinib, estramustine, etoposide phosphate, etoposide, everolimus, exemestane, fentanyl citrate, filgrastim, floxuridine, fludarabine, fluorouracil, fruquintinib, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, ixabepilone, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, paclitaxel albumin-stabilized nanoparticle formulation, pamidronate, panitumumab, pegaspargase, pegfilgrastim, pemetrexed disodium, pentostatin, pertuzuma, pipobroman, plicamycin, procarbazine, quinacrine, rasburicase, rituximab, sorafenib, streptozocin, sulfatinib, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, volitinib, vorinostat, and zoledronate.

In particular embodiments, compounds according to the disclosure are used in combination with one or more anti-cancer agent selected from methotrexate, paclitaxel albumin-stabilized nanoparticle formulation, ado-trastuzumab emtansine, eribulin, doxorubicin, fluorouracil, everolimus, anastrozole, pamidronate disodium, exemestane, capecitabine, cyclophosphamide, docetaxel, epirubicin, toremifene, fulvestrant, letrozole, gemcitabine, gemcitabine hydrochloride, goserelin acetate, trastuzumab, ixabepilone, lapatinib ditosylate, megestrol acetate, tamoxifen citrate, pamidronate disodium, palbociclib, and pertuzumab for the treatment of breast cancer.

Other anti-cancer agents/drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclin-dependent kinase inhibitors; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitors; microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; zanoterone; zilascorb; zinostatin stimalamer; 5-fluorouracil; and leucovorin.

In some embodiments, the anti-cancer agent/drug is an agent that stabilizes microtubules. As used herein, a “microtubulin stabilizer” means an anti-cancer agent/drug which acts by arresting cells in the G2-M phases due to stabilization of microtubules. Examples of microtubulin stabilizers include ACLITAXEL® and Taxol® analogues. Additional examples of microtubulin stabilizers include without limitation the following marketed drugs and drugs in development: Discodermolide (also known as NVP—XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone AN-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

In another embodiment, the anti-cancer agent/drug is an agent that inhibits microtubules. As used herein, a “microtubulin inhibitor” means an anti-cancer agent which acts by inhibiting tubulin polymerization or microtubule assembly. Examples of microtubulin inhibitors include without limitation the following marketed drugs and drugs in development: Erbulozole (also known as R-55104); Dolastatin 10 (also known as DLS-10 and NSC-376128); Mivobulin isethionate (also known as CI-980); Vincristine; NSC-639829; ABT-751 (Abbott, also known as E-7010); Altorhyrtins (such as Altorhyrtin A and Altorhyrtin C); Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9); Cemadotin hydrochloride (also known as LU-103793 and NSC-D-669356); Auristatin PE (also known as NSC-654663); Soblidotin (also known as TZT-1027), LS-4559-P (Pharmacia, also known as LS-4577); LS-4578 (Pharmacia, also known as LS-477-P); LS-4477 (Pharmacia), LS-4559 (Pharmacia); RPR-112378 (Aventis); Vincristine sulfate; DZ-3358 (Daiichi); GS-164 (Takeda); GS-198 (Takeda); KAR-2 (Hungarian Academy of Sciences); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (also known as LY-355703); Vitilevuamide; Tubulysin A; Canadensol; Centaureidin (also known as NSC-106969); T-138067 (Tularik, also known as T-67, TL-138067 and TI-138067); COBRA-1 (Parker Hughes Institute, also known as DDE-261 and WHI-261); H10 (Kansas State University); H16 (Kansas State University); Oncocidin A1 (also known as BTO-956 and DIME); DDE-313 (Parker Hughes Institute); SPA-2 (Parker Hughes Institute); SPA-1 (Parker Hughes Institute, also known as SPIKET-P); 3-IAABU (Cytoskeleton/Mt. Sinai School of Medicine, also known as M1F-569); Narcosine (also known as NSC-5366); Nascapine, D-24851 (Asta Medica), A-105972 (Abbott); Hemiasterlin; 3-BAABU (Cytoskeleton/Mt. Sinai School of Medicine, also known as M1F-191); TMPN (Arizona State University); Vanadocene acetylacetonate; T-138026 (Tularik); Monsatrol; Inanocine (also known as NSC-698666); 3-IAABE (Cytoskeleton/Mt. Sinai School of Medicine); A-204197 (Abbott); T-607 (Tularik, also known as T-900607); RPR-115781 (Aventis); Eleutherobins (such as Desmethyleleutherobin, Desaetyleleutherobin, Isoeleutherobin A, and Z-Eleutherobin); Halichondrin B; D-64131 (Asta Medica); D-68144 (Asta Medica); Diazonamide A; A-293620 (Abbott); NPI-2350 (Nereus); TUB-245 (Aventis); A-259754 (Abbott); Diozostatin; (−)-Phenylahistin (also known as NSCL-96F037); D-68838 (Asta Medica); D-68836 (Asta Medica); Myoseverin B; D-43411 (Zentaris, also known as D-81862); A-289099 (Abbott); A-318315 (Abbott); HTI-286 (also known as SPA-110, trifluoroacetate salt) (Wyeth); D-82317 (Zentaris); D-82318 (Zentaris); SC-12983 (NCI); Resverastatin phosphate sodium; BPR—OY-007 (National Health Research Institutes); SSR-250411 (Sanofi); Combretastatin A4; eribulin (Halaven®); and analogs and derivatives thereof.

In further embodiments, compounds according to the disclosure are used in combination with one or more alkylating agents, antimetabolites, natural products, or hormones.

Examples of alkylating agents useful in the methods of the disclosure include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.).

Examples of antimetabolites useful in the methods of the disclosure include but are not limited to folic acid analog (e.g., methotrexate), or pyrimidine analogs (e.g., fluorouracil, floxouridine, cytarabine), and purine analogs (e.g., mercaptopurine, thioguanine, pentostatin). Examples of natural products useful in the methods of the disclosure include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin) or enzymes (e.g., L-asparaginase).

Examples of hormones and antagonists useful for the treatment of cancer include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), and gonadotropin releasing hormone analog (e.g., leuprolide).

Other agents that can be used in combination with the compounds of the disclosure for the treatment of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), and adrenocortical suppressant (e.g., mitotane, aminoglutethimide). Other anti-cancer agents/drugs that can be used in combination with the compounds of the disclosure include, but are not limited to, liver X receptor (LXR) modulators, including LXR agonists and LXR beta-selective agonists; aryl hydrocarbon receptor (AhR) inhibitors; inhibitors of the enzyme poly ADP ribose polymerase (PARP), including olaparib, iniparib, rucaparib, veliparib; inhibitors of vascular endothelial growth factor (VEGF) receptor tyrosine kinases, including cediranib; programmed cell death protein 1 (PD-1) inhibitors, including nivolumab (Bristol-Myers Squibb Co.) and pembrolizumab (Merck & Co., Inc.; MK-3475); MEK inhibitors, including cobimetinib; B-Raf enzyme inhibitors, including vemurafenib; cytotoxic T lymphocyte antigen (CTLA-4) inhibitors, including tremelimumab; programmed death-ligand 1 (PD-L1) inhibitors, including MEDI4736 (AstraZeneca); inhibitors of the Wnt pathway; inhibitors of epidermal growth factor receptor (EGFR) including AZD9291 (AstraZeneca), erlotinib, gefitinib, panitumumab, and cetuximab; adenosine A2A receptor inhibitors; adenosine A2B receptor inhibitors; colony-stimulating factor-1 receptor (CSF1R) inhibitors, including PLX3397 (Plexxikon), and inhibitors of CD73.

The compounds of the disclosure can be used in combination with one or more therapeutic strategies including immune checkpoint inhibitors, including inhibitors of PD-1, PD-L1, and CTLA-4.

The compounds of the disclosure can be used in combination with one or more anti-cancer agents selected from MCL-1 inhibitors, e.g., homoharringtonin (HHT) and omacetaxine; BCL-2 inhibitors, e.g., venetoclax (ABT-199), navitoclax (ABT-263), ABT-737, gossypol (AT-101), apogossypolone (ApoG2) and obatoclax; selective inhibitors of nuclear export (SINEs), e.g., selinexor (KPT-330).

In particular embodiments, the compounds of the disclosure are used in combination with one or more anti-cancer agents selected from methotrexate (Abitrexate®; Folex®; Folex PFS®; Mexate®; Mexate-AQ®); nelarabine (Arranon®); blinatumomab (Blincyto®); rubidomycin hydrochloride or daunorubicin hydrochloride (Cerubidine®); cyclophosphamide (Clafen®; Cytoxan®; Neosar®); clofarabine (Clofarex®; Clolar®); cytarabine (Cytosar-U®; Tarabine PFS®); dasatinib (Sprycel®); doxorubicin hydrochloride; asparaginase Erwinia chrysanthemi (Erwinaze); imatinib mesylate (Gleevec®); ponatinib hydrochloride (Iclusig®); mercaptopurine (Purinethol; Purixan); pegaspargase (Oncaspar®); prednisone; vincristine sulfate (Oncovin®, Vincasar PFS®, Vincrex®); vincristine sulfate liposome (Marqibo®); hyper-CVAD (fractionated cyclophosphamide, vincristine, adriamycin, and dexamethasone); arsenic trioxide (Trisenox®); idarubicin hydrochloride (Idamycin®); mitoxantrone hydrochloride; thioguanine (Tabloid®); ADE (cytarabine, daunorubicin, and etoposide); alemtuzumab (Lemtrada®, Campath®); chlorambucil (Ambochlorin®, Amboclorin®, Leukeran®, Linfolizin®); ofatumumab (Arzerra®); bendamustine hydrochloride (Treanda®); fludarabine phosphate (Fludara®); obinutuzumab (Gazyva®); ibrutinib (Imbruvica®); idelalisib (Zydelig®); mechlorethamine hydrochloride (Mustargen®); rituximab (Rituxan®); chlorambucil-prednisone; CVP (cyclophosphamide, vincristine, and prednisone); bosutinib (Bosulif®); busulfan (Busulfex®; Myleran®); omacetaxine mepesuccinate (Synribo®); nilotinib (Tasigna®); Intron® A (recombinant interferon Alfa-2b); DOT1L inhibitors, including EPZ-5676 (Epizyme, Inc.); and inhibitors of bromodomain and extra-terminal motif (BET) proteins (BET inhibitors), including MS417, JQ1, I-BET 762, and I-BET 151 for the treatment of leukemia.

Compounds of the disclosure can be used in combination with one or more other agents or therapies for the treatment of insulin resistance, pre-diabetes, diabetes (e.g., Type 2 diabetes or Type 1 diabetes), and risk of diabetes, including but not limited to insulins and insulin analogues, such as Humulin® (Eli Lilly), Lantus® (Sanofi Aventis), Novolin® (Novo Nordisk), and Exubera® (Pfizer); Avandamet® (metformin HCl and rosiglitazone maleate, GSK); Avandaryl® (glimepiride and rosiglitazone maleate, GSK); Metaglip® (glipizide and metformin HCl, Bristol Myers Squibb); Glucovance® (glyburide and metformin HCl, Bristol Myers Squibb); PPAR gamma agonists, such as Avandia® (rosiglitizone maleate, GSK) and Actos® (pioglitazone hydrochloride, Takeda/Eli Lilly); sulfonylureas, such as Amaryl® (glimepiride, Sanofi Aventis), Diabeta® (glyburide, Sanofi Aventis), Micronase®/Glynase® (glyburide, Pfizer), and Glucotrol®/Glucotrol XL® (glipizide, Pfizer); meglitinides, such as Prandin®/NovoNorm® (repaglinide, Novo Nordisk), Starlix® (nateglinide, Novartis), and Glufast® (mitiglinide, Takeda); biguanides, such as Glucophase®/Glucophase XR® (metformin HCl, Bristol Myers Squibb) and Glumetza® (metformin HCl, Depomed); thiazolidinediones; amylin analogs; GLP-1 analogs; DPP-IV inhibitors such as Januvia® (sitagliptin, Merck) and Galvus® (vildagliptin, Novartis); PTB-1 B inhibitors; protein kinase inhibitors (including AMP-activated protein kinase inhibitors); glucagon antagonists, glycogen synthase kinase-3 beta inhibitors; glucose-6-phoshatase inhibitors; glycogen phosphorylase inhibitors; sodium glucose co-transporter inhibitors; and alpha-glucosidase inhibitors, such as Glycet® (miglitol, Pfizer); statins, fibrates, and Zetia® (ezetimibe); alpha-blockers; beta-blockers; calcium channel blockers; diuretics; angiotensin converting enzyme (ACE) inhibitors; dual ACE and neutral endopeptidase (NEP) inhibitors; angiotensin-receptor blockers (ARBs); aldosterone synthase inhibitors; aldosterone-receptor antagonists; endothelin receptor antagonists; orlistat; phentermine; sibutramine; Acomplia® (rimonabant); thiazolidinediones (e.g., rosiglitazone, pioglitazone); SGLT 2 inhibitors (e.g., dapagliflozin, remogliflozin etabonate, sergliflozin, canagliflozin, and 1-chloro-4-(β-D-glucopyranos-1-yl)-2-[4-((′S)-tetrahydrofuran-3-yloxy)-benzyl]-benzene); PPAR-gamma-agonists (e.g., Gl 262570) and antagonists; PPAR-gamma/alpha modulators (e.g., KRP 297); alpha-glucosidase inhibitors (e.g., acarbose, voglibose); DPPIV inhibitors (e.g., Januvia® (sitagliptin), Galvus®/Zomelis® (vildagliptin), Onglyza® (saxagliptin), Nesina®/Vipidia® (alogliptin), and Tradjenta®/Trajenta® (linagliptin)); alpha2-antagonists; glucagon-like protein-1 (GLP-1) receptor agonists and analogues (e.g., exendin-4); amylin; inhibitors of protein tyrosinephosphatase 1; substances that affect deregulated glucose production in the liver, e.g., inhibitors of glucose-6-phosphatase, or fructose-1,6-bisphosphatase, glycogen phosphorylase; glucagon receptor antagonists; inhibitors of phosphoenol pyruvate carboxykinase; glycogen synthase kinase and glucokinase activators; lipid lowering agents such as HMG-CoA-reductase inhibitors (e.g., simvastatin, atorvastatin); fibrates (e.g., bezafibrate, fenofibrate), nicotinic acid and the derivatives thereof, PPAR-alpha agonists, PPAR-delta agonists; ACAT inhibitors (e.g., avasimibe); cholesterol absorption inhibitors such as ezetimibe; bile acid-binding substances such as cholestyramine; inhibitors of ileac bile acid transport; HDL-raising compounds such as CETP inhibitors and ABC1 regulators; active substances for treating obesity such as sibutramine and tetrahydrolipostatin; SDRIs; axokine; leptin; leptin mimetics; antagonists of the cannabinoid 1 receptor; and MCH-1 receptor antagonists; MC4 receptor agonists; NPY5 and NPY2 antagonists; beta3 adrenergic agonists such as SB—418790 and AD-9677; agonists of the 5HT2c receptor; GABA-receptor antagonists; Na-channel blockers; topiramate; protein-kinase C inhibitors; advanced glycation end product inhibitors; and aldose reductase inhibitors.

### Pharmaceutical Formulations, Administration, and Dosage Forms

When employed as pharmaceuticals, the compounds of the disclosure can be administered in the form of a pharmaceutical composition which refers to a combination of a compound of the disclosure, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, one or more of the compounds of the disclosure above in combination with one or more pharmaceutically acceptable carriers. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Compounds or compositions described herein may be administered to a patient using any amount and any route of administration effective for treating or lessening the severity of one or more of the diseases and conditions described herein. The exact amount required will vary from subject to subject, depending on the species, age, and general condition of the subject, the severity of the infection, disease or disorder, the particular agent, its mode of administration, and the like. Provided compounds are preferably formulated in a particular unit dosage form for ease of administration and uniformity of dosage. The expression “unit dosage form” as used herein refers to a physically discrete unit of agent appropriate for the patient to be treated.

The therapeutic dosage of the compounds of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. The dosage is likely to depend on such variables as the type and extent of progression of the disease or disorder, the overall health status of the particular patient, the relative biological efficacy of the compound selected, formulation of the excipient, and its route of administration.

### Numbered Embodiments A

A1. A compound of Formula I,

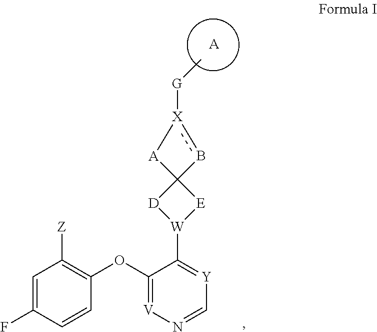

a stereoisomer, or a pharmaceutically acceptable salt thereof, wherein:
[symbol] denotes a single bond or a double bond, as valency permits;
A, B, D, and E are each independently selected from —O—, —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent, O, CH₂, or NH, wherein
   when G is absent, X is connected with a nitrogen of Ring A;
   when G is O, CH₂, or NH, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
Z is Cy², halo, C₁₋₆ alkyl, C₁₋₆ haloalkyl, C₁₋₆ cyanoalkyl, C₂₋₆ alkenyl, C₂₋₆ alkynyl, CN, NO₂, OR^{a3}, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3}, NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, and P(O)R^{c3}R^{d3} wherein said C₁₋₆ alkyl, C₁₋₆ haloalkyl, C₁₋₆ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3} NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH;
R^{z} is H, C₁₋₆ alkyl, or phenyl;
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5} NR^{e5}C(O)R^{b5}, NR^{e5}C(O)OR^{a5}, NR^{e5}C(O)NR^{c5}R^{d5}, NR^{c5}S(O)R^{b5}, NR^{c5}S(O)₂R^{b5}, NR^{c5}S(O)₂NR^{c5}R^{d5} S(O)R^{b5}, S(O)NR^{c5}R^{d5}, S(O)₂R^{b5}, and S(O)₂NR^{c5}R^{d5}, wherein said C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{e5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs}
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{a3}, R^{b3}, R^{c3}, R^{d3}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R³;
each R^{e3} and R^{e5} is independently selected from H, C₁₋₆ alkyl, and CN;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   A2. The compound of Numbered Embodiment A1, a stereoisomer, or a pharmaceutically acceptable salt thereof, wherein when [symbol] is a double bond, X is C.
   A3. The compound of any one of the preceding Numbered Embodiments, a stereoisomer, or a pharmaceutically acceptable salt thereof, wherein the spiro moiety represented by the below formula:
[symbol] wherein e and f indicate points of attachment to the remainder of the molecule, is selected from:

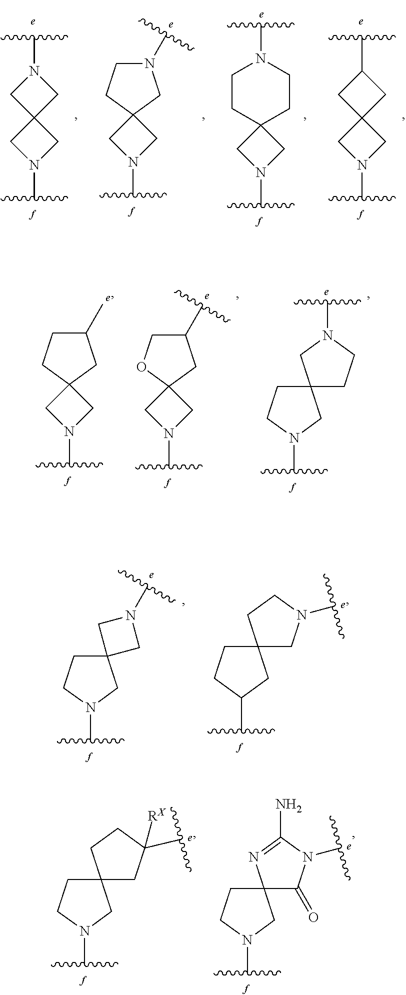

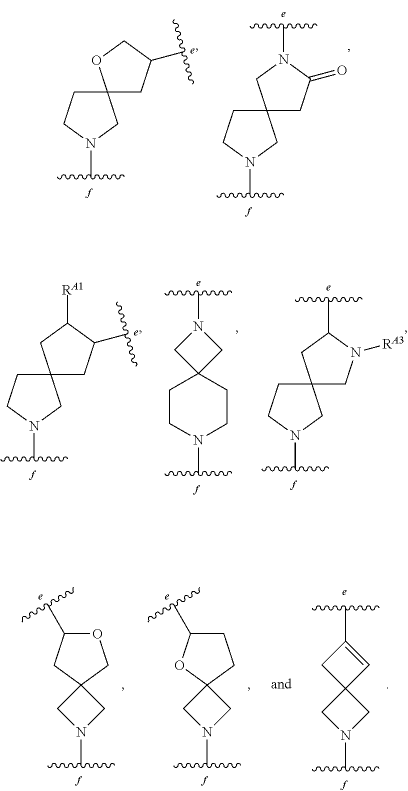

A4. A compound of Formula II,

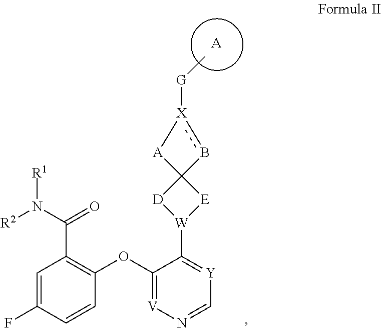

a stereoisomer, or a pharmaceutically acceptable salt thereof, wherein:
[symbol] denotes a single bond or a double bond, as valency permits;
A, B, D, and E are each independently selected from —O—, —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH;
R^{z} is H, C₁₋₆ alkyl, or phenyl;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}, N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   A5. A compound of Formula II-a,

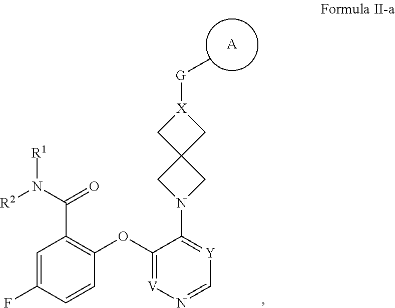

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}, N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   A6. A compound of Formula II-b,

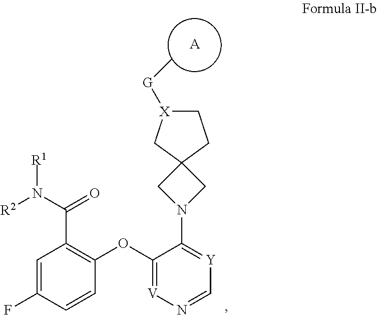

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   A7. A compound of Formula II-c,

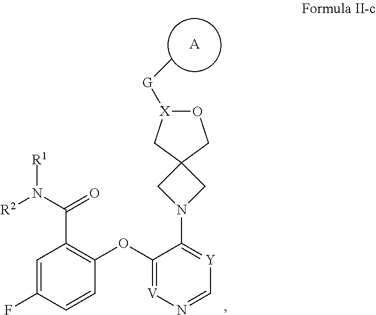

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   A8. A compound of Formula II-d,

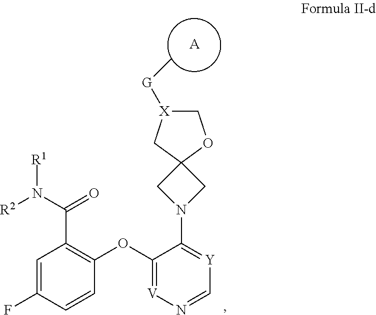

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a};
   each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   A9. A compound of Formula II-e,

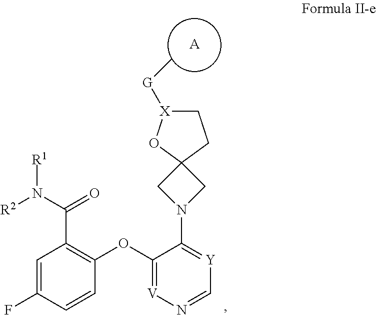

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C2-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   A10. The compound of any one of the preceding Numbered Embodiments, wherein V is N.
   A11. The compound of any one of the preceding Numbered Embodiments, wherein V is CH.
   A12. The compound of any one of the preceding Numbered Embodiments, wherein Y is N.
   A13. The compound of any one of the preceding Numbered Embodiments, wherein Y is CH.
   A14. The compound of any one of the preceding Numbered Embodiments, wherein X is N.
   A15. The compound of any one of the preceding Numbered Embodiments, wherein X is CR^{X} and wherein R^{X} is absent, H, halo, CN, OH, C₁₋₆ alkyl, C₁₋₆ alkoxy, amino, C₁₋₆ alkyl amino, or C₂₋₈ dialkylamino.
   A16. The compound of any one of the preceding Numbered Embodiments, wherein G is absent and X is connected with a nitrogen of Ring A.
   A17. The compound of any one of the preceding Numbered Embodiments, wherein G is absent and X is CH which is connected with a nitrogen of Ring A.
   A18. The compound of any one of the preceding Numbered Embodiments, wherein G is O and G is connected with the nitrogen of Ring A.
   A19. The compound of any one of the preceding Numbered Embodiments, wherein G is CH₂ and G is connected with the nitrogen of Ring A.
   A20. The compound of any one of the preceding Numbered Embodiments, wherein G is NH and G is connected with the nitrogen of Ring A.
   A21. The compound of any one of the preceding Numbered Embodiments, wherein W is N.
   A22. The compound of any one of the preceding Numbered Embodiments, wherein W is CR^{W}, and wherein R^{W} is H, halo, CN, OH, C₁₋₆ alkyl, C₁₋₆ alkoxy, amino, C₁₋₆ alkyl amino, or C₂₋₈ dialkylamino;
   A23. The compound of any one of the preceding Numbered Embodiments, wherein R¹ is C₁₋₆ alkyl.
   A24. The compound of any one of the preceding Numbered Embodiments, wherein R¹ is ethyl.
   A25. The compound of any one of the preceding Numbered Embodiments, wherein R¹ is isopropyl.
   A26. The compound of any one of the preceding Numbered Embodiments, wherein R¹ is C₁₋₆ haloalkyl.
   A27. The compound of any one of the preceding Numbered Embodiments, wherein R¹ is —CH₂—CHF₂.
   A28. The compound of any one of the preceding Numbered Embodiments, wherein R¹ is C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′) OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.
   A29. The compound of any one of the preceding Numbered Embodiments, wherein R¹ is

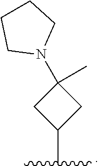 [SMILES: *C1CC(C)(N2CCCC2)C1]

A30. The compound of any one of the preceding Numbered Embodiments, wherein R¹ is

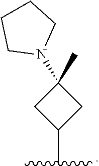

A31. The compound of any one of the preceding Numbered Embodiments, wherein R¹ and R² form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy.
A32. The compound of any one of the preceding Numbered Embodiments, wherein R¹ and R² form a 6-membered heterocycloalkyl with the nitrogen to which they are connected, wherein the heterocycloalkyl is substituted with one or more C₁₋₆ alkyl, halo, OH, CN, or C₁₋₆ alkoxy.
A33. The compound of any one of the preceding Numbered Embodiments, wherein R² is C₁₋₆ alkyl.
A34. The compound of any one of the preceding Numbered Embodiments, wherein R² is isopropyl.
A35. The compound of any one of the preceding Numbered Embodiments, wherein R¹ and R² form

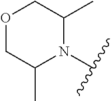 [SMILES: *N1C(C)COCC1C]

group with the nitrogen to which they are connected.
A36. The compound of any one of the preceding Numbered Embodiments, wherein R¹ and R² form

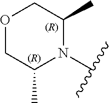

group with the nitrogen to which they are connected.
A37. The compound of any one of the preceding Numbered Embodiments, wherein Ring A is 4-18 membered heterocycloalkyl having at least one nitrogen and optionally one or more other heteroatom ring members, wherein the 4-18 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.
   A38. The compound of any one of the preceding Numbered Embodiments, wherein Ring A is

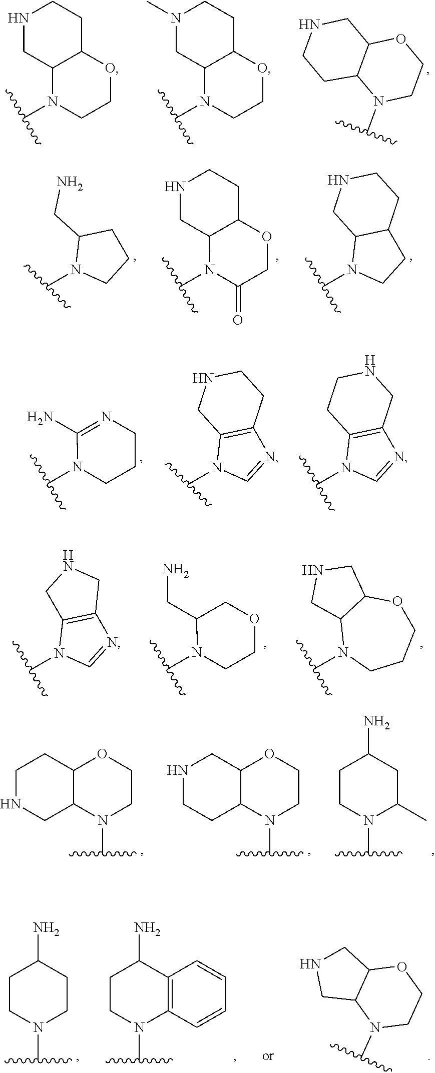

A39. The compound of any one of the preceding Numbered Embodiments, wherein Ring A is

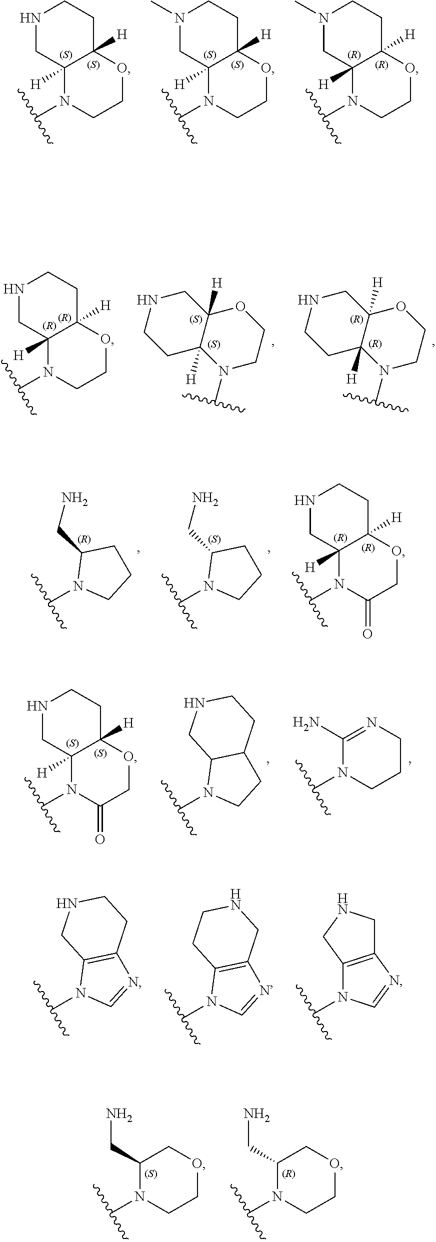

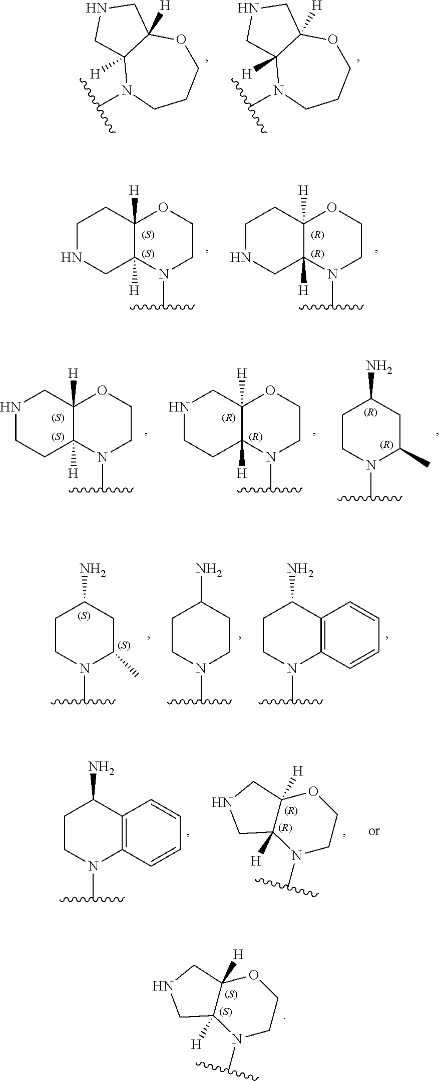

A40. The compound of any one of the preceding Numbered Embodiments, wherein Ring A is 5- to 10-membered heteroaryl having at least one nitrogen and optionally one or more other heteroatom ring members, wherein the 5- to 10-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.
   A41. The compound of any one of the preceding Numbered Embodiments, wherein Ring A is

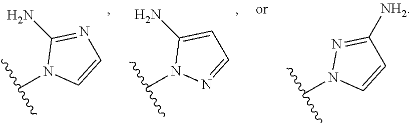 [SMILES: *n1ccnc1N ; *n1nccc1N ; *n1ccc(N)n1]

A42. The compound of any one of the preceding Numbered Embodiments, wherein each R³ is independently H, methyl, —CH₂—NH₂, oxo, or —NH₂.
A43. A compound as shown in Table 1 or a stereoisomer thereof, or a pharmaceutically acceptable salt thereof.
A44. A compound as shown in Table 1 or a pharmaceutically acceptable salt thereof.
A45. A compound as shown in Table 1.
A46. A compound as shown in Table 1 or a pharmaceutically acceptable salt thereof, wherein the salt is hydrochloride.
A47. The compound according to any one of the preceding Numbered Embodiments, wherein the compound is useful for the treatment of cancer and wherein the compound minimizes hERG binding.
A48. A pharmaceutical composition comprising a compound of any one of the preceding Numbered Embodiments, or a pharmaceutically acceptable salt thereof, and at least one pharmaceutically acceptable carrier.
A49. A pharmaceutical composition comprising a salt or crystalline form of the compound of any one of Numbered Embodiments A1-A47, and at least one pharmaceutically acceptable carrier.
A50. A method of inhibiting the interaction between menin and MLL comprising contacting the menin and MLL with a compound of any one of Numbered Embodiments A1-A47 or a pharmaceutical composition of Numbered Embodiment A48 or Numbered Embodiment A49.
A51. A method of treating cancer in a patient comprising administering to the patient a compound of any one of Numbered Embodiments A1-A47 or a pharmaceutical composition of Numbered Embodiment A48 or Numbered Embodiment A49.
A52. The method of Numbered Embodiment A51, wherein the cancer is a hematological cancer.
A53. The method of Numbered Embodiment A51, wherein the cancer is a leukemia.
A54. The method of Numbered Embodiment A51, wherein the cancer is a lymphoma.
A55. The method of Numbered Embodiment A51, wherein the cancer is mixed lineage leukemia (MLL), MLL-related leukemia, MLL-associated leukemia, MLL-positive leukemia, MLL-induced leukemia, rearranged mixed lineage (KMT2A-rearranged) leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
A56. The method of Numbered Embodiment A51, wherein the cancer is an abstract nucleophosmin (NPM1)-mutated acute myeloid leukemia (i.e., NPM1^{mut} acute myloid leukemia).
A57. The method of Numbered Embodiment A51, wherein the cancer is a rearranged mixed lineage (KMT2A-rearranged) leukemia (MLL-r).
A58. The compound of any one of Numbered Embodiments A1-A47 or a pharmaceutically acceptable salt thereof, or the pharmaceutical composition of Numbered Embodiment A48 or Numbered Embodiment A49, for use in treating or preventing a disease caused by, or associated with, menin expression, activity, and/or function.
A59. The compound of any one of Numbered Embodiments A1-A47 or a pharmaceutically acceptable salt thereof, or the pharmaceutical composition of Numbered Embodiment A48 or Numbered Embodiment A49, for use in treating or preventing cancer.
A60. Use of the compound of any one of Numbered Embodiments A1-A47 or a pharmaceutically acceptable salt thereof, or the pharmaceutical composition of Numbered Embodiment A48 or Numbered Embodiment A49, for treating or preventing a disease caused by, or associated with, menin expression, activity, and/or function.
A61. Use of the compound of any one of Numbered Embodiments A1-A47 or a pharmaceutically acceptable salt thereof, or the pharmaceutical composition of Numbered Embodiment A48 or Numbered Embodiment A49, in the manufacture of a medicament for treating or preventing a disease caused by, or associated with, menin expression, activity, and/or function.
A62. Use of a compound of any of Numbered Embodiments A1-A47 or a pharmaceutically acceptable salt thereof, or the pharmaceutical composition of Numbered Embodiment A48 or Numbered Embodiment A49, for treating or preventing cancer.
A63. Use of the compound of any one of Numbered Embodiments A1-A47 or a pharmaceutically acceptable salt thereof, or the pharmaceutical composition of Numbered Embodiment A48 or Numbered Embodiment A49, in the manufacture of a medicament for treating or preventing cancer.
A64. A kit comprising the compound of any one of Numbered Embodiments A1-A47 or a pharmaceutically acceptable salt thereof, or the pharmaceutical composition of Numbered Embodiment A48 or Numbered Embodiment A49 and instructions for its use.
A65. A method of preparing a compound herein according to a scheme of the present disclosure or synthetic description in the Examples.
A66. An intermediate useful in the preparation of any one of the compounds herein.
A67. The compound of any one of the preceding Numbered Embodiments, wherein R¹ is

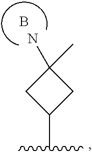

wherein Ring B is a 3- to 12-membered heterocycloalkyl which has at least one nitrogen and optionally one or more other heteroatom ring members, wherein the heterocycloalkyl is optionally substituted.
A68. The compound of any one of the preceding Numbered Embodiments, wherein R¹ is

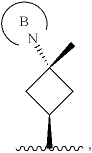

wherein Ring B is a 3- to 12-membered heterocycloalkyl which has at least one nitrogen and optionally one or more other heteroatom ring members, wherein the heterocycloalkyl is optionally substituted.
A69. The compound of any one of the preceding Numbered Embodiments, wherein R¹ is

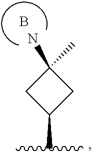

wherein Ring B is a 3- to 12-membered heterocycloalkyl which has at least one nitrogen and optionally one or more other heteroatom ring members, wherein the heterocycloalkyl is optionally substituted.

### Numbered Embodiments B

B1. A compound of Formula I,

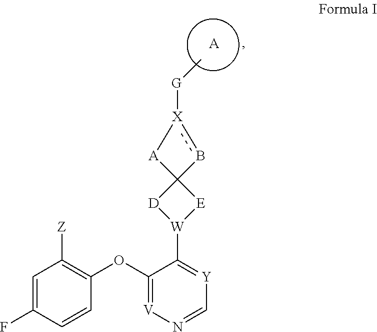

a stereoisomer, or a pharmaceutically acceptable salt thereof, wherein:
[symbol] denotes a single bond or a double bond, as valency permits;
A, B, D, and E are each independently selected from —O—, —C(R^{A1})(R^{A2})—, —C(R^{A1})(R^{A2})—C(R^{A1})(R^{A2})—, —C(R^{A1})(R^{A2})—O—, —C(R^{A1})(R^{A2})—NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent, 0, CH₂, or NH, wherein
   when G is absent, X is connected with a nitrogen of Ring A;
   when G is O, CH₂, or NH, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
Z is Cy², halo, C₁₋₆ alkyl, C₁₋₆ haloalkyl, C₁₋₆ cyanoalkyl, C₂₋₆ alkenyl, C₂₋₆ alkynyl, CN, NO₂, OR^{a3}, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3}, NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, S(O)₂NR^{c3}R^{d3}, and P(O)R^{c3}R^{d3} wherein said C₁₋₆ alkyl, C₁₋₆ haloalkyl, C₁₋₆ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3} NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH;
R^{z} is H, C₁₋₆ alkyl, or phenyl;
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5} NR⁵C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5} S(O)R^{b5}, S(O)NR^{c5}R^{d5}, S(O)₂R^{b5}, and S(O)₂NR^{c5}R^{d5}, wherein said C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs}
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{a3}, R^{b3}, R^{c3}, R^{d3}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R³;
each R^{e3} and R^{e5} is independently selected from H, C₁₋₆ alkyl, and CN;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and each R_{N} is independently H, C₁₋₆ alkyl, or C₁₋₆ haloalkyl;
wherein any cycloalkyl or heterocycloalkyl group is optionally further substituted with 1 or 2 oxo groups.
   B2. The compound of Numbered Embodiment B1, a stereoisomer, or a pharmaceutically acceptable salt thereof, wherein when [symbol] is a double bond, X is C.
   B3. The compound of any one of Numbered Embodiments B1-B2, a stereoisomer, or a pharmaceutically acceptable salt thereof, wherein the spiro moiety represented by the below formula:

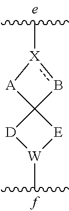

wherein e and f indicate points of attachment to the remainder of the molecule, is selected from:

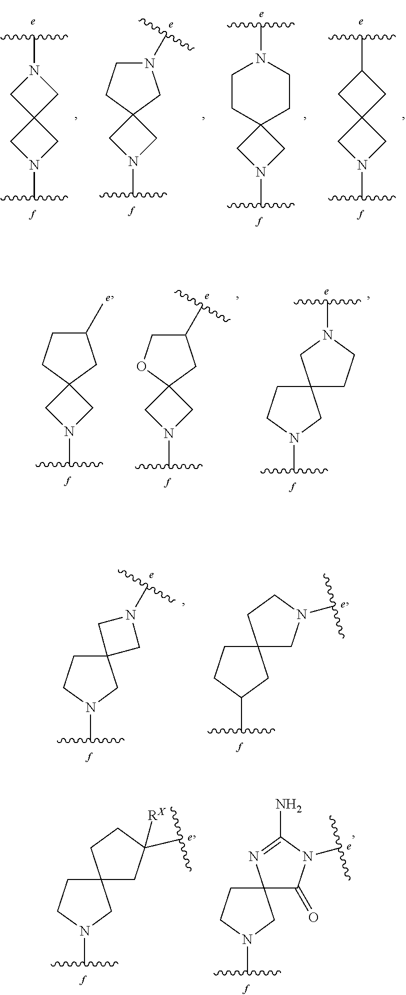

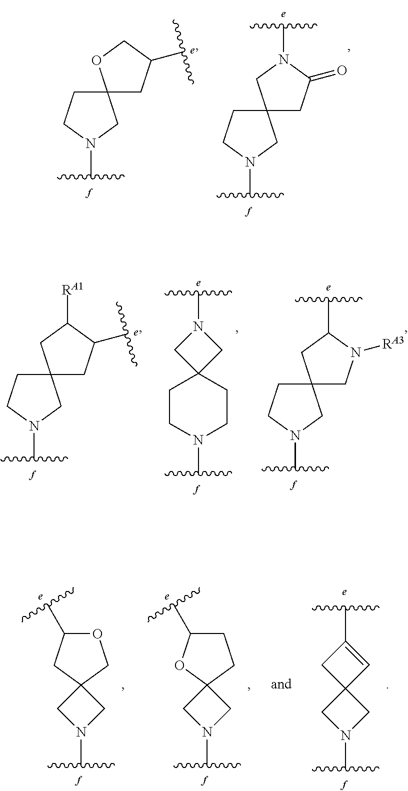

B4. A compound of Formula II,

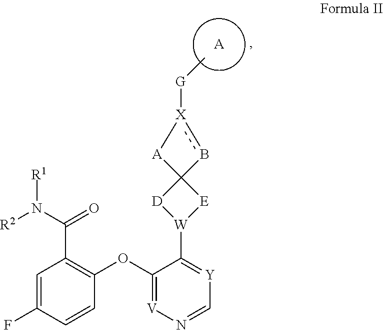

a stereoisomer, or a pharmaceutically acceptable salt thereof, wherein:
[symbol] denotes a single bond or a double bond, as valency permits;
A, B, D, and E are each independently selected from —O—, —C(R^{A1})(R^{A2})—, —C(R^{A1})(R^{A2})—C(R^{A1})(R^{A2})—, —C(R^{A1})(R^{A2})—O—, —C(R^{A1})(R^{A2})NR^{A3}—, —C(═O)—, —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH;
R^{z} is H, C₁₋₆ alkyl, or phenyl;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   B5. A compound of Formula II-a,

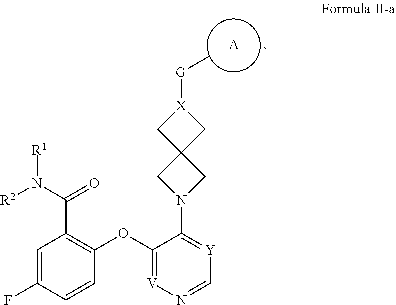

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and each R_{N} is independently H, C₁₋₆ alkyl, or C₁₋₆ haloalkyl;
wherein any cycloalkyl or heterocycloalkyl group is optionally further substituted with 1 or 2 oxo groups.
   B6. A compound of Formula II-b,

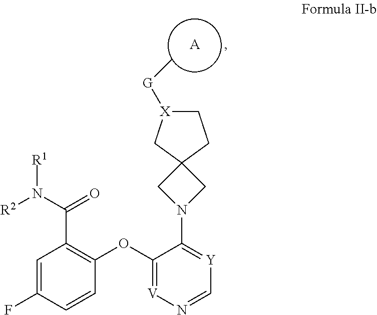

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   B7. A compound of Formula II-c,

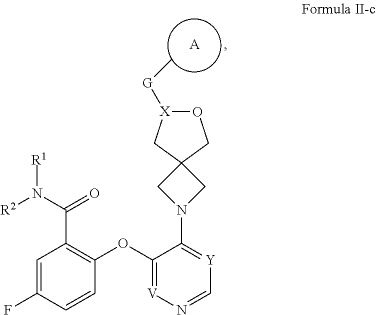

a stereoisomer, or a pharmaceutically acceptable salt thereof, wherein:
V is Nor C H;
Y is Nor C H;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and each R_{N} is independently H, C₁₋₆ alkyl, or C₁₋₆ haloalkyl;
wherein any cycloalkyl or heterocycloalkyl group is optionally further substituted with 1 or 2 oxo groups.
   B8. A compound of Formula II-d,

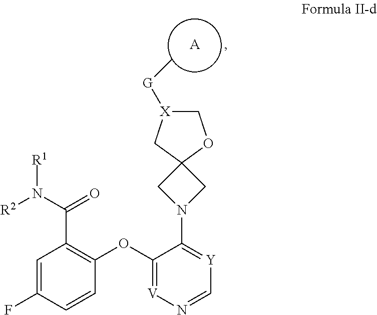

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   B9. A compound of Formula II-e,

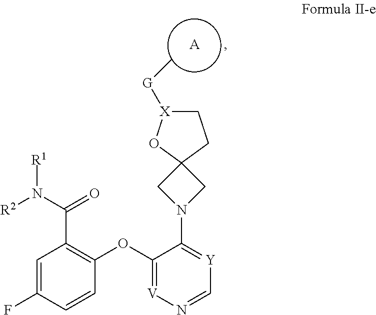

a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
   when G is absent, X is connected with a nitrogen of Ring A;
   when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
   (a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
   (b) 3- to 12-membered heterocycloalkyl comprising a sulfur atom, wherein the sulfur atom of the 3- to 12-membered heterocycloalkyl is optionally substituted with one or two R^{1s};
   each R^{1s} is independently oxo or ═NR^{4a};
   (c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
   (d) 3- to 12-membered heterocycloalkyl which has at least one nitrogen and optionally one or more other heteroatom ring members, wherein the 3- to 12-membered heterocycloalkyl is optionally substituted with one or more R^{1bs};
   each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
   (e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
   each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
   each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
   each R^{3b}′ is independently H, C₁₋₆ alkyl, C₁₋₆ haloalkyl, C₃₋₁₂ cycloalkyl, or 3- to 12-membered heterocycloalkyl;
   each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.
   B10. The compound of any one of Numbered Embodiments B1-B9, wherein V is N.
   B11. The compound of any one of Numbered Embodiments B1-B10, wherein V is CH.
   B12. The compound of any one of Numbered Embodiments B1-B11, wherein Y is N.
   B13. The compound of any one of Numbered Embodiments B1-B12, wherein Y is CH.
   B14. The compound of any one of Numbered Embodiments B1-B13, wherein X is N.
   B15. The compound of any one of Numbered Embodiments B1-B14, wherein X is CR^{X} and wherein R^{X} is absent, H, halo, CN, OH, C₁₋₆ alkyl, C₁₋₆ alkoxy, amino, C₁₋₆ alkyl amino, or C₂₋₈ dialkylamino.
   B16. The compound of any one of Numbered Embodiments B1-B15, wherein G is absent and X is connected with a nitrogen of Ring A.
   B17. The compound of any one of Numbered Embodiments B1-B16, wherein G is absent and X is CH which is connected with a nitrogen of Ring A.
   B18. The compound of any one of Numbered Embodiments B1-B17, wherein G is O and G is connected with the nitrogen of Ring A.
   B19. The compound of any one of Numbered Embodiments B1-B18, wherein G is CH₂ and G is connected with the nitrogen of Ring A.
   B20. The compound of any one of Numbered Embodiments B1-B19, wherein G is NH and G is connected with the nitrogen of Ring A.
   B21. The compound of any one of Numbered Embodiments B1-B20, wherein W is N.
   B22. The compound of any one of Numbered Embodiments B1-B21, wherein W is CR^{W}, and wherein R^{W} is H, halo, CN, OH, C₁₋₆ alkyl, C₁₋₆ alkoxy, amino, C₁₋₆ alkyl amino, or C₂₋₈ dialkylamino; B23. The compound of any one of Numbered Embodiments B1-B22, wherein R¹ is C₁₋₆ alkyl.
   B24. The compound of any one of Numbered Embodiments B1-B23, wherein R¹ is ethyl.
   B25. The compound of any one of Numbered Embodiments B1-B24, wherein R¹ is isopropyl.
   B26. The compound of any one of Numbered Embodiments B1-B25, wherein R¹ is C₁₋₆ haloalkyl.
   B27. The compound of any one of Numbered Embodiments B1-B26, wherein R¹ is —CH₂—CHF₂.
   B28. The compound of any one of Numbered Embodiments B1-B27, wherein R¹ is C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′) OR^{3a}′, or S(═O)₂R^{3a}′, wherein the C₁₋₆ alkyl or C₁₋₆ haloalkyl is optionally substituted with one or more C₁₋₆ alkoxy;
each R^{3a}, is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.
   B29. The compound of any one of Numbered Embodiments B1-B28, wherein R¹ is

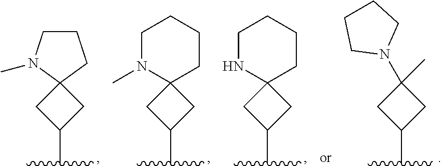

B30. The compound of any one of Numbered Embodiments B1-B29, wherein R¹ is

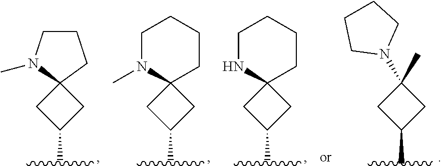

B31. The compound of any one of Numbered Embodiments B1-B30, wherein R¹ and R² form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy.

B32. The compound of any one of Numbered Embodiments B1-B31, wherein R¹ and R² form a 6-membered heterocycloalkyl with the nitrogen to which they are connected, wherein the heterocycloalkyl is substituted with one or more C₁₋₆ alkyl, halo, OH, CN, or C₁₋₆ alkoxy.
B33. The compound of any one of Numbered Embodiments B1-B32, wherein R² is C₁₋₆ alkyl.
B34. The compound of any one of Numbered Embodiments B1-B33, wherein R² is isopropyl.
B35. The compound of any one of Numbered Embodiments B1-B34, wherein R¹ and R² form

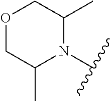 [SMILES: *N1C(C)COCC1C]

group with the nitrogen to which they are connected.
B36. The compound of any one of Numbered Embodiments B1-B35, wherein R¹ and R² form

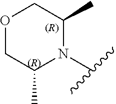

group with the nitrogen to which they are connected.
B37. The compound of any one of Numbered Embodiments B1-B36, wherein Ring A is 4-18 membered heterocycloalkyl having at least one nitrogen and optionally one or more other heteroatom ring members, wherein the 4-18 membered heterocycloalkyl is optionally substituted with one or more R³.
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.
   B38. The compound of any one of Numbered Embodiments B1-B37, wherein Ring A is

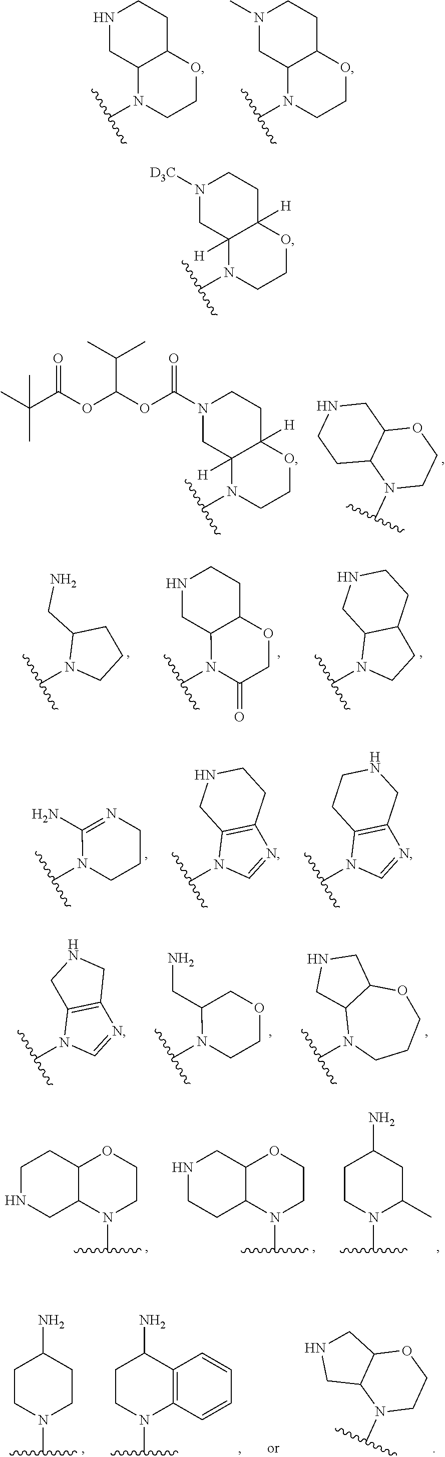

B39. The compound of any one of Numbered Embodiments B1-B38, wherein Ring A is

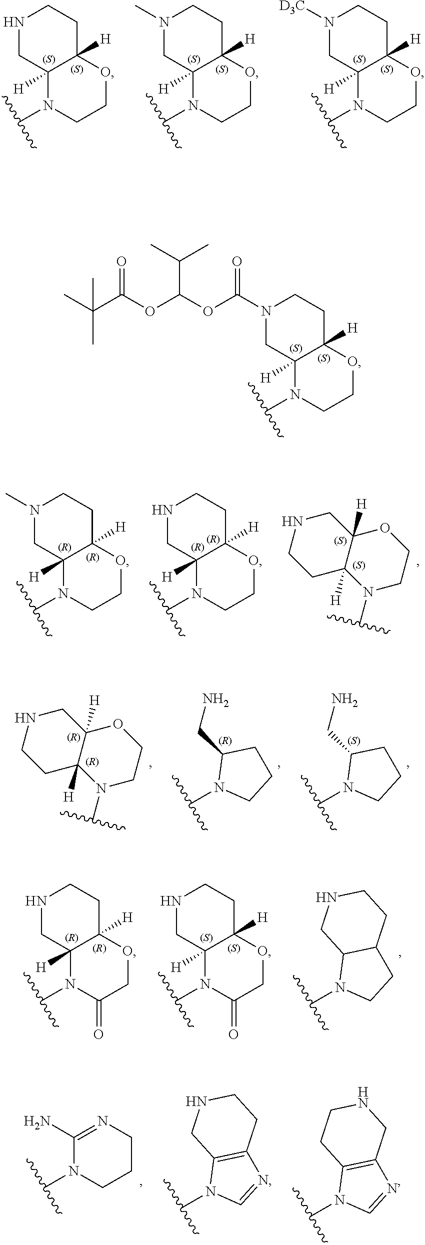

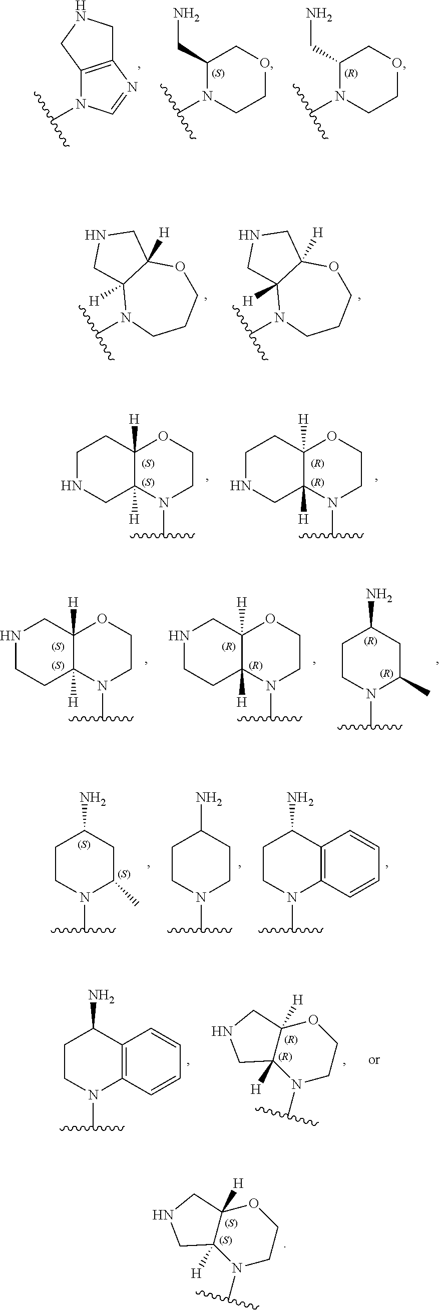

B40. The compound of any one of Numbered Embodiments B1-B39, wherein Ring A is 5- to 10-membered heteroaryl having at least one nitrogen and optionally one or more other heteroatom ring members, wherein the 5- to 10-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.
   B41. The compound of any one of Numbered Embodiments B1-B40, wherein Ring A is

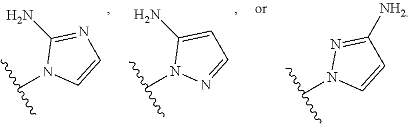 [SMILES: *n1ccnc1N ; *n1nccc1N ; *n1ccc(N)n1]

B42. The compound of any one of Numbered Embodiments B1-B41, wherein each R³ is independently H, methyl, —CH₂—NH₂, oxo, or —NH₂.
B43. A compound as shown in Table 1 or a stereoisomer thereof, or a pharmaceutically acceptable salt thereof.
B44. A compound as shown in Table 1 or a pharmaceutically acceptable salt thereof.
B45. A compound as shown in Table 1.
B46. A compound as shown in Table 1 or a pharmaceutically acceptable salt thereof, wherein the salt is hydrochloride.
B47. The compound according to any one of Numbered Embodiments B1-B46, wherein the compound is useful for the treatment of cancer and wherein the compound minimizes hERG binding.
B48. A pharmaceutical composition comprising a compound of any one of Numbered Embodiments B1-B47, or a pharmaceutically acceptable salt thereof, and at least one pharmaceutically acceptable carrier.
B49. A pharmaceutical composition comprising a salt or crystalline form of the compound of any one of Numbered Embodiments B1-B47, and at least one pharmaceutically acceptable carrier.
B50. A method of inhibiting the interaction between menin and MLL comprising contacting the menin and MLL with a compound of any one of Numbered Embodiments B1-B47 or a pharmaceutical composition of Numbered Embodiment B48 or Numbered Embodiment B49.
B51. A method of treating cancer in a patient comprising administering to the patient a compound of any one of Numbered Embodiments B1-B47 or a pharmaceutical composition of Numbered Embodiment B48 or Numbered Embodiment B49.
B52. The method of Numbered Embodiment B51, wherein the cancer is a hematological cancer.
B53. The method of Numbered Embodiment B51, wherein the cancer is a leukemia.
B54. The method of Numbered Embodiment B51, wherein the cancer is a lymphoma.
B55. The method of Numbered Embodiment B51, wherein the cancer is mixed lineage leukemia (MLL), MLL-related leukemia, MLL-associated leukemia, MLL-positive leukemia, MLL-induced leukemia, rearranged mixed lineage (KMT2A-rearranged) leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CMIL), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
B56. The method of Numbered Embodiment B51, wherein the cancer is an abstract nucleophosmin (NPM1)-mutated acute myeloid leukemia (i.e., NPM1^{mut} acute myloid leukemia).
B57. The method of Numbered Embodiment B51, wherein the cancer is a rearranged mixed lineage (KMT2A-rearranged) leukemia (MLL-r).
B58. The compound of any one of Numbered Embodiments B1-B47 or a pharmaceutically acceptable salt thereof, or the pharmaceutical composition of Numbered Embodiment B48 or Numbered Embodiment B49, for use in treating or preventing a disease caused by, or associated with, menin expression, activity, and/or function.
B59. The compound of any one of Numbered Embodiments B1-B47 or a pharmaceutically acceptable salt thereof, or the pharmaceutical composition of Numbered Embodiment B48 or Numbered Embodiment B49, for use in treating or preventing cancer.
B60. Use of the compound of any one of Numbered Embodiment B1-B47 or a pharmaceutically acceptable salt thereof, or the pharmaceutical composition of Numbered Embodiment B48 or Numbered Embodiment B49, for treating or preventing a disease caused by, or associated with, menin expression, activity, and/or function.
B61. Use of the compound of any one of Numbered Embodiment B1-B47 or a pharmaceutically acceptable salt thereof, or the pharmaceutical composition of Numbered Embodiment B48 or Numbered Embodiment B49, in the manufacture of a medicament for treating or preventing a disease caused by, or associated with, menin expression, activity, and/or function.
B62. Use of a compound of any of Numbered Embodiments B1-B47 or a pharmaceutically acceptable salt thereof, or the pharmaceutical composition of Numbered Embodiment B48 or Numbered Embodiment B49, for treating or preventing cancer.
B63. Use of the compound of any one of Numbered Embodiment B1-B47 or a pharmaceutically acceptable salt thereof, or the pharmaceutical composition of Numbered Embodiment B48 or Numbered Embodiment B49, in the manufacture of a medicament for treating or preventing cancer.
B64. A kit comprising the compound of any one of Numbered Embodiment B1-B47 or a pharmaceutically acceptable salt thereof, or the pharmaceutical composition of Numbered Embodiment B48 or Numbered Embodiment B49 and instructions for its use.
B65. A method of preparing a compound herein according to a scheme of the present disclosure or synthetic description in the Examples.
B66. An intermediate useful in the preparation of any one of the compounds herein.
B67. The intermediate of Numbered Embodiment B66, wherein the intermediate is of Formula X-I:

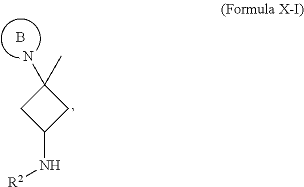

a stereoisomer, or a salt thereof, wherein:
Ring B is a 3- to 12-membered heterocycloalkyl which has at least one nitrogen and optionally one or more other heteroatom ring members, wherein the heterocycloalkyl is optionally substituted.
   B68. The intermediate of Numbered Embodiment B66, wherein the intermediate is of Formula X-I:

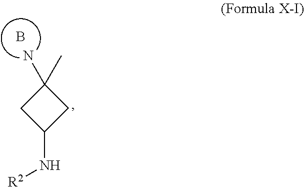

or a salt thereof, wherein:
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
Ring B is a 3- to 12-membered heterocycloalkyl which has at least one nitrogen and optionally one or more other heteroatom ring members, wherein the heterocycloalkyl is optionally substituted by one or more substituents selected from Cy², halo, C₁₋₆ alkyl, C₁₋₆ haloalkyl, C₁₋₆ cyanoalkyl, C₂₋₆ alkenyl, C₂₋₆ alkynyl, CN, NO₂, OR^{a3}, SR^{a3}, C(O)R^{b3}C(O)NR¹R², C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3}, NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, S(O)₂NR^{c3}R^{d3}, and P(O)R^{c3}R^{d3} wherein said C₁₋₆ alkyl, C₁₋₆ haloalkyl, C₁₋₆ cyanoalkyl, C₂₋₆ alkenyl, and C₂₋₆ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3}, NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3} S(O)₂R^{b3}, and S(O)₂NR^{c3}R^{d3};
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2},
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{c5})NR^{c5}R^{d5}, NR^{c5}C(═NR^{c5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5} NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5}, wherein said C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
each R^{a3}, R^{b3}, R^{c3}, R^{d3}, R^{a5}, R^{b5}, R^{c5}, and R^{d5} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, —C₆₋₁₀ aryl-C₁₋₆ alkyl, —C₃₋₁₀ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl.
   B69. The intermediate of Numbered Embodiment B66, wherein the intermediate is of Formula X-I:

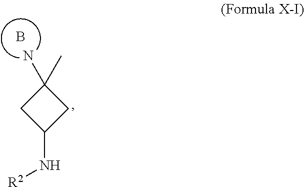

a stereoisomer, or a salt thereof, wherein:
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g};
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
Ring B is a 3- to 12-membered heterocycloalkyl which has at least one nitrogen and optionally one or more other heteroatom ring members, wherein the heterocycloalkyl is optionally substituted with one or more C₁₋₆ alkyl, halo, OH, CN, or C₁₋₆ alkoxy.
   B70. The intermediate of Numbered Embodiment B66, wherein the intermediate is of Formula X-I:

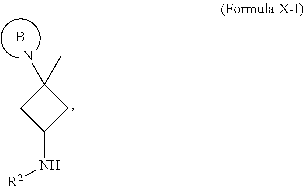

a stereoisomer, or a salt thereof, wherein:
R² is H, C₁₋₆ alkyl, C₁₋₆ haloalkyl, C₁₋₆ alkoxy, C₂₋₆ alkenyl, or C₂₋₆ alkynyl;
Ring B is a 3- to 12-membered heterocycloalkyl which has at least one nitrogen and optionally one or more other heteroatom ring members, wherein the heterocycloalkyl is optionally substituted with one or more C₁₋₆ alkyl, halo, OH, CN, or C₁₋₆ alkoxy.
   B71. The intermediate of Numbered Embodiment B66, wherein the intermediate is of Formula X-I:

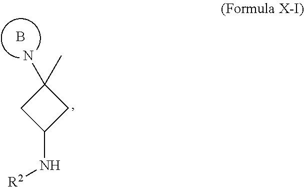

a stereoisomer, or a salt thereof, wherein:
R² is H, C₁₋₆ alkyl, C₁₋₆ haloalkyl, C₁₋₆ alkoxy, C₂₋₆ alkenyl, or C₂₋₆ alkynyl;
Ring B is a 4- to 7-membered heterocycloalkyl which has at least one nitrogen and optionally one or more other heteroatom ring members, wherein the heterocycloalkyl is optionally substituted with one or more C₁₋₆ alkyl, halo, OH, CN, or C₁₋₆ alkoxy.
   B72. The intermediate of Numbered Embodiment B66, wherein the intermediate is of Formula X-I:

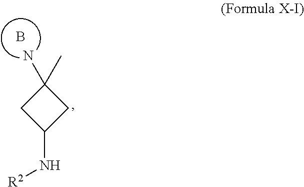

a stereoisomer, or a salt thereof, wherein:
R² is C₁₋₆ alkyl;
Ring B is a 4- to 7-membered heterocycloalkyl containing one nitrogen atom, wherein the heterocycloalkyl is optionally substituted with one or more C₁₋₆ alkyl, halo, OH, CN, or C₁₋₆ alkoxy.
   B73. The intermediate of Numbered Embodiment B66, wherein the intermediate is of Formula X-I:

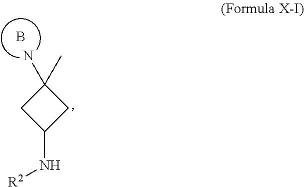

a stereoisomer, or a salt thereof, wherein:
R² is C₁₋₆ alkyl;
Ring B is a 4- to 7-membered heterocycloalkyl containing one nitrogen atom.
   B74. The intermediate of Numbered Embodiment B66, wherein the intermediate is of Formula 5-B, 5-C, 5-D, 5-E, 5-F, or 5-H:

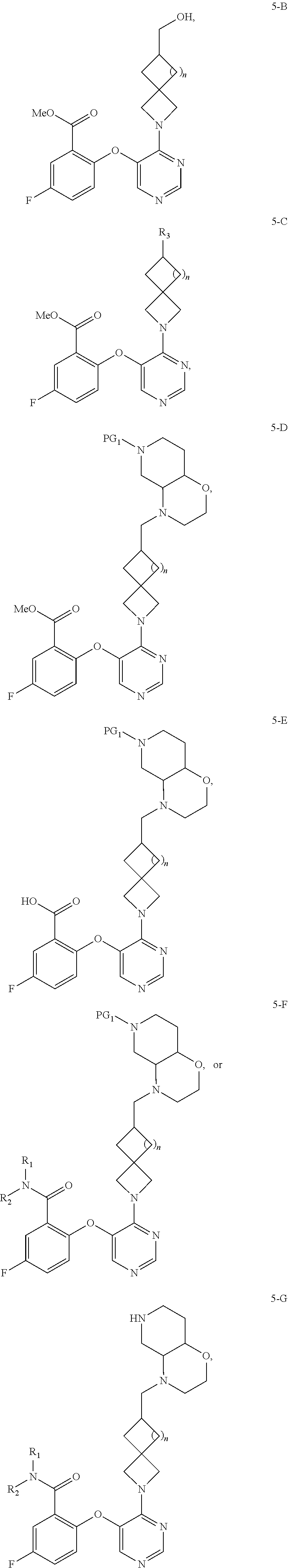

a stereoisomer, or a salt thereof, wherein:
R¹ and R² are as defined in any one of Numbered Embodiments B1-B73;
PG¹ is an amine protecting group;
R³ is CHO,

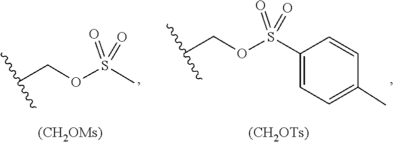

or CH₂—I; and
n is 1 or 2.
   B75. The intermediate of Numbered Embodiment B74, a stereoisomer, or a salt thereof, wherein:
R¹ and R² are as defined in any one of Numbered Embodiments B1-B73;
PG₁ is an amine protecting group selected from benzyloxycarbonyl (Cbz) and tert-butyloxycarbonyl (Boc);
R³ is CHO,

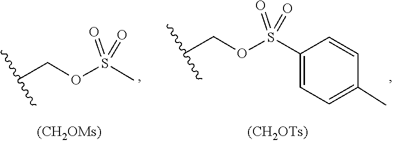

or CH₂—I; and
n is 1 or 2.
   B76. The intermediate of Numbered Embodiment B75, wherein the intermediate is of Formula 5-D, 5-E, 5-F, or 5-G, a stereoisomer, or a salt thereof, wherein:
R¹ and R² are as defined in any one of Numbered Embodiments B1-B72;
PG₁ is an amine protecting group selected from benzyloxycarbonyl (Cbz) and tert-butyloxycarbonyl (Boc);
R³ is CHO,

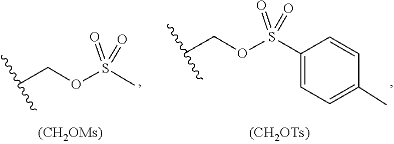

or CH₂—I; and
n is 1 or 2.
   B77. The intermediate of any one of Numbered Embodiments B66-B73, wherein the intermediate is:

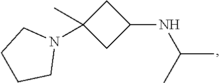

a stereoisomer, or a salt thereof.
B78. The intermediate of any one of Numbered Embodiments B66-B73, wherein the intermediate is:

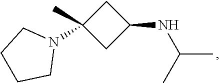

or a salt thereof.
B79. The intermediate of any one of Numbered Embodiments B66-B73, wherein the intermediate is:

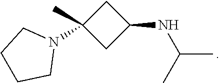

B80. The intermediate of any one of Numbered Embodiments B66 or B74-B76, wherein the intermediate is selected from:

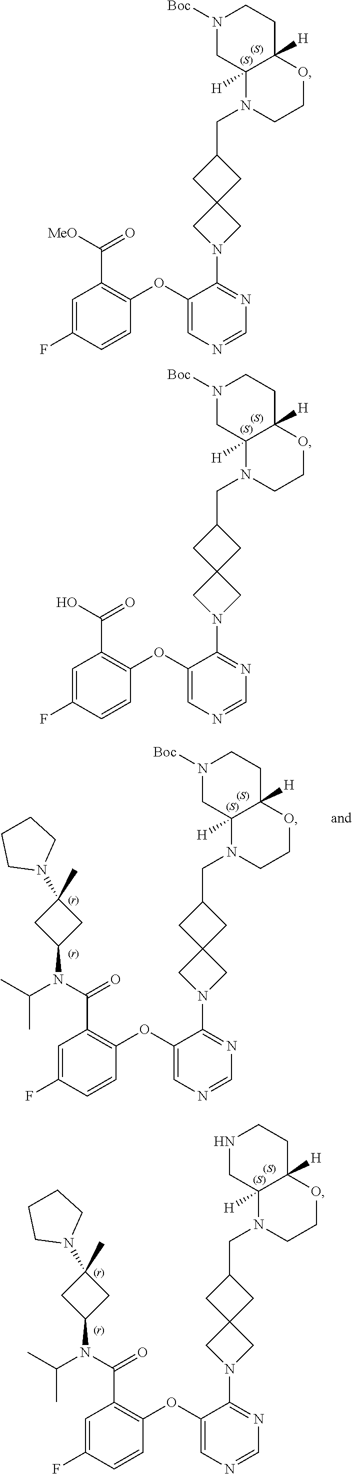

or a salt thereof.
B81. The intermediate of Numbered Embodiment B66, wherein the intermediate is selected from:

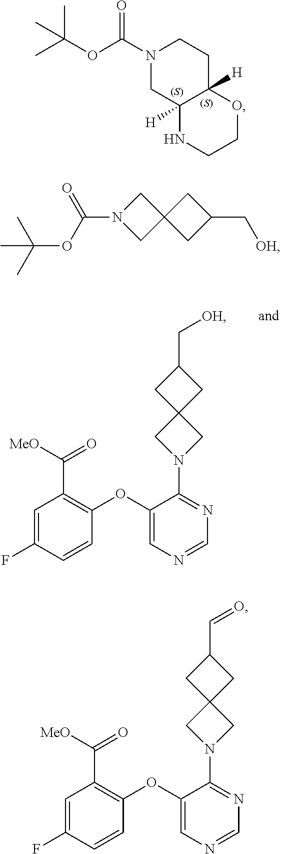

or a salt thereof.
B82. The intermediate of Numbered Embodiment B66, wherein the intermediate is selected from:

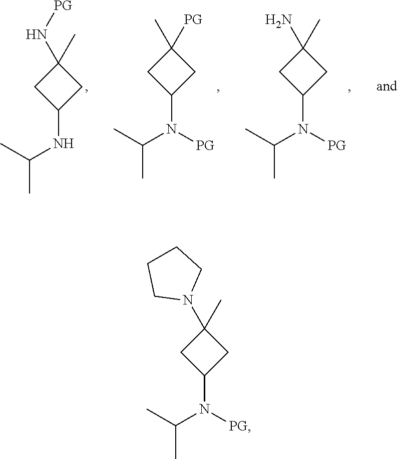

wherein each PG is independently an amine protecting group.
B83. The intermediate of Numbered Embodiment B82, wherein each PG is an amine protecting group independently selected from benzyloxycarbonyl (Cbz) and tertbutyloxycarbonyl (Boc).
B84. The intermediate of any one of Numbered Embodiments B82-B83, wherein the intermediate is selected from:

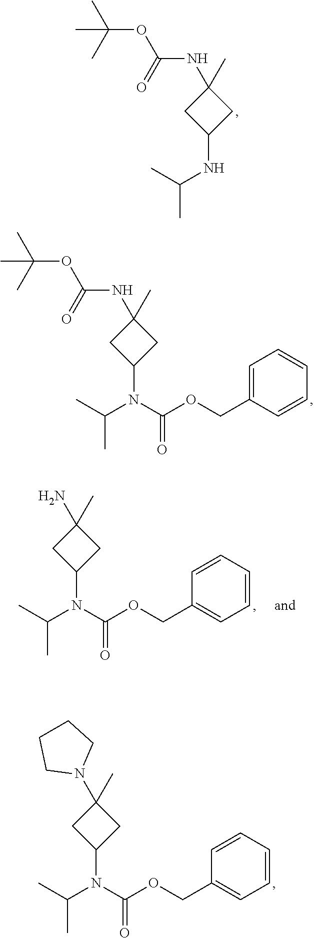 [SMILES: CC(C)NC1CC(C)(NC(=O)OC(C)(C)C)C1 ; CC(C)N(C(=O)OCc1ccccc1)C1CC(C)(NC(=O)OC(C)(C)C)C1 ; CC(C)N(C(=O)OCc1ccccc1)C1CC(C)(N)C1 ; CC(C)N(C(=O)OCc1ccccc1)C1CC(C)(N2CCCC2)C1]

a stereoisomer, or a salt thereof.
B85. The intermediate of any one of Numbered Embodiments B82-B84, wherein the intermediate is selected from:

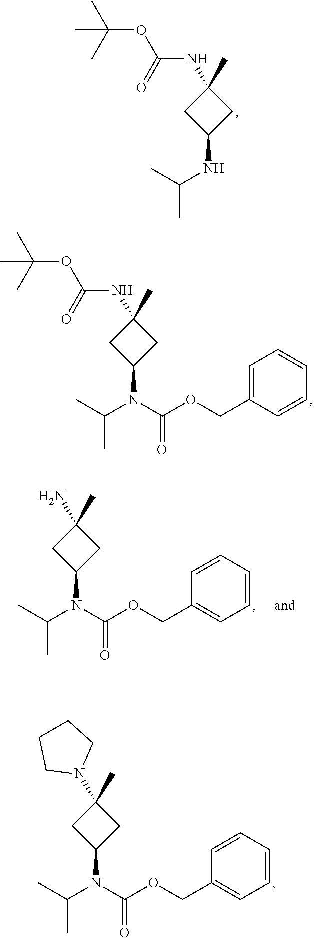 [SMILES: CC(C)N[C@H]1C[C@](C)(NC(=O)OC(C)(C)C)C1 ; CC(C)N(C(=O)OCc1ccccc1)[C@H]1C[C@](C)(NC(=O)OC(C)(C)C)C1 ; CC(C)N(C(=O)OCc1ccccc1)[C@H]1C[C@](C)(N)C1 ; CC(C)N(C(=O)OCc1ccccc1)[C@H]1C[C@](C)(N2CCCC2)C1]

or a salt thereof.
B86. The compound of any one Numbered Embodiments B1-B46, wherein the compound is selected from Compound Nos. 1, 2, 3, 13, 14, 15, 16, 24, 25, 41, 42, 43, 61, 62, 63, and 64, a stereoisomer, or a pharmaceutically acceptable salt thereof.
B87. The compound of any one Numbered Embodiments B1-B46, wherein the compound is selected from Compound Nos. 1, 2, 3, 13, 14, 15, 16, 24, 25, 41, 42, 43, 61, 62, 63, and 64, or a pharmaceutically acceptable salt thereof.
B88. The compound of Numbered Embodiment B86 or B87, wherein the salt is a hydrochloride.
B89. The compound of any one Numbered Embodiments B1-B46, wherein the compound is selected from Compound Nos. 1, 2, 3, 13, 14, 15, 16, 24, 25, 41, 42, 43, 61, 62, 63, and 64.
B90. The compound of any one Numbered Embodiments B1-B46, wherein the compound is Compound No. 1, a stereoisomer, or a pharmaceutically acceptable salt thereof.
B91. The compound of any one Numbered Embodiments B1-B46, wherein the compound is Compound No. 2, a stereoisomer, or a pharmaceutically acceptable salt thereof.
B92. The compound of any one Numbered Embodiments B1-B46, wherein the compound is Compound No. 3, a stereoisomer, or a pharmaceutically acceptable salt thereof.
B93. The compound of any one Numbered Embodiments B1-B46, wherein the compound is Compound No. 13, a stereoisomer, or a pharmaceutically acceptable salt thereof.
B94. The compound of any one Numbered Embodiments B1-B46, wherein the compound is Compound No. 14, a stereoisomer, or a pharmaceutically acceptable salt thereof.
B95. The compound of any one Numbered Embodiments B1-B46, wherein the compound is Compound No. 15, a stereoisomer, or a pharmaceutically acceptable salt thereof.
B96. The compound of any one Numbered Embodiments B1-B46, wherein the compound is Compound No. 16, a stereoisomer, or a pharmaceutically acceptable salt thereof.
B97. The compound of any one Numbered Embodiments B1-B46, wherein the compound is Compound No. 24, a stereoisomer, or a pharmaceutically acceptable salt thereof.
B98. The compound of any one Numbered Embodiments B1-B46, wherein the compound is Compound No. 25, a stereoisomer, or a pharmaceutically acceptable salt thereof.
B99. The compound of any one Numbered Embodiments B1-B46, wherein the compound is Compound No. 41, a stereoisomer, or a pharmaceutically acceptable salt thereof.
B100. The compound of any one Numbered Embodiments B1-B46, wherein the compound is Compound No. 42, a stereoisomer, or a pharmaceutically acceptable salt thereof.
B101. The compound of any one Numbered Embodiments B1-B46, wherein the compound is Compound No. 43, a stereoisomer, or a pharmaceutically acceptable salt thereof.
B102. The compound of any one Numbered Embodiments B1-B46, wherein the compound is Compound No. 61, a stereoisomer, or a pharmaceutically acceptable salt thereof.
B103. The compound of any one Numbered Embodiments B1-B46, wherein the compound is Compound No. 62, a stereoisomer, or a pharmaceutically acceptable salt thereof.
B104. The compound of any one Numbered Embodiments B1-B46, wherein the compound is Compound No. 63, a stereoisomer, or a pharmaceutically acceptable salt thereof.
B105. The compound of any one Numbered Embodiments B1-B46, wherein the compound is Compound No. 64, a stereoisomer, or a pharmaceutically acceptable salt thereof.
B106. The compound according to any one of Numbered Embodiments B1-B46 or B86-B105, wherein the compound is useful for the treatment of cancer and wherein the compound minimizes hERG binding.
B107. A pharmaceutical composition comprising a compound of any one of Numbered Embodiments B1-B47 or B86-B106, or a pharmaceutically acceptable salt thereof, and at least one pharmaceutically acceptable carrier.
B108. A pharmaceutical composition comprising a salt or crystalline form of the compound of any one of Numbered Embodiments B1-B47 or B86-B106, and at least one pharmaceutically acceptable carrier.
B109. A method of inhibiting the interaction between menin and MLL comprising contacting the menin and MLL with a compound of any one of Numbered Embodiments B1-B47 or B86-B106 or a pharmaceutical composition of Numbered Embodiment B107 or Numbered Embodiment B108.
B110. A method of treating cancer in a patient comprising administering to the patient a compound of any one of Numbered Embodiments B1-B47 or B86-B106 or a pharmaceutical composition of Numbered Embodiment B107 or Numbered Embodiment B108.
B111. The method of Numbered Embodiment B109, wherein the cancer is a hematological cancer.
B112. The method of Numbered Embodiment B109, wherein the cancer is estrogen-receptor positive breast cancer.
B113. The method of Numbered Embodiment B109, wherein the cancer is a leukemia.
B114. The method of Numbered Embodiment B109, wherein the cancer is a lymphoma.
B115. The method of Numbered Embodiment B109, wherein the cancer is mixed lineage leukemia (MLL), MLL-related leukemia, MLL-associated leukemia, MLL-positive leukemia, MLL-induced leukemia, rearranged mixed lineage (KMT2A-rearranged) leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CMIL), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.
B116. The method of Numbered Embodiment B109, wherein the cancer is an abstract nucleophosmin (NPM1)-mutated acute myeloid leukemia (i.e., NPM1^{mut} acute myloid leukemia).
B117. The method of Numbered Embodiment B109, wherein the cancer is a rearranged mixed lineage (KMT2A-rearranged) leukemia (MLL-r).
B118. The compound of any one of Numbered Embodiments B1-B47 or B86-B106, or a pharmaceutically acceptable salt thereof, or the pharmaceutical composition of Numbered Embodiment B107 or Numbered Embodiment B108, for use in treating or preventing a disease caused by, or associated with, menin expression, activity, and/or function.
B119. The compound of any one of Numbered Embodiments B1-B47 or B86-B106, or a pharmaceutically acceptable salt thereof, or the pharmaceutical composition of Numbered Embodiment B107 or Numbered Embodiment B108, for use in treating or preventing cancer.
B120. Use of the compound of any one of Numbered Embodiments B1-B47 or B86-B106, or a pharmaceutically acceptable salt thereof, or the pharmaceutical composition of Numbered Embodiment B107 or Numbered Embodiment B108, for treating or preventing a disease caused by, or associated with, menin expression, activity, and/or function.
B121. Use of the compound of any one of Numbered Embodiments B1-B47 or B86-B106, or a pharmaceutically acceptable salt thereof, or the pharmaceutical composition of Numbered Embodiment B107 or Numbered Embodiment B108, in the manufacture of a medicament for treating or preventing a disease caused by, or associated with, menin expression, activity, and/or function.
B122. Use of a compound of any of Numbered Embodiments B1-B47 or B86-B106, or a pharmaceutically acceptable salt thereof, or the pharmaceutical composition of Numbered Embodiment B107 or Numbered Embodiment B108, for treating or preventing cancer.
B123. Use of the compound of any one of Numbered Embodiments B1-B47 or B86-B106, or a pharmaceutically acceptable salt thereof, or the pharmaceutical composition of Numbered Embodiment B107 or Numbered Embodiment B108, in the manufacture of a medicament for treating or preventing cancer.
B124. A kit comprising the compound of any one of Numbered Embodiments B1-B47 or B86-B106, or a pharmaceutically acceptable salt thereof, or the pharmaceutical composition of Numbered Embodiment B107 or Numbered Embodiment B108 and instructions for its use.

### EXAMPLES

As depicted in the Examples below, the compounds of the present disclosure were prepared by the methods described below, Although the Examples illustrate specific methods, those of skill in the art will recognize that other reasonable approaches might also be utilized to prepare these and related compounds.

| Abbreviation | Definition |
|---|---|
| ACN | Acetonitrile |
| AcOH | Acetic acid |
| (Boc)₂O | Di-tert-butyl dicarbonate |
| t-BuOH | tert-Butanol |
| Cbz-Cl | Benzyl chloroformate; benzyl chloridocarbonate |
| DBU | 1,8-Diazabicyclo[5.4.0]undec-7-ene |
| DCE | 1,2-Dichloroethane |
| DCM | Dichloromethane (methylene chloride) |
| DIAD | Diisopropyl azodicarboxylate |
| DIEA (DIPEA), | Diisopropylethylamine |
| iPr₂NEt | |
| DMA (DMAc) | Dimethylacetamide |
| DMAP | 4-Dimethylaminopyridine |
| DMF | Dimethylformamide |
| DMSO | Dimethylsulfoxide |
| dppf | 1,1-bis(Diphenylphosphino)ferrocene |
| DSC | Differential scanning calorimetry |
| EDC•HCl | N-Ethyl-N′-(3-dimethylaminopropyl)carbodiimide |
| | hydrochloride |
| Et₃N (TEA) | Triethylamine |
| EtOAc | Ethyl acetate |
| EtOH | Ethanol |
| h | Hour(s) |
| HATU | 1-[bis(Dimethylamino)methylene]-1H-1,2,3- |
| | triazolo[4,5-b]pyridinium 3- |
| | oxide hexafluorophosphate |
| HBTU | 2-(1H-Benzotriazole-1-yl)-1,1,3,3-tetramethyluronium |
| | hexafluorophosphate |
| HOBt | 1-Hydroxybenzonotrazole |
| HPLC | High performance liquid chromatography |
| IPA, i-PrOH | Isopropyl alcohol |
| LCMS | Liquid chromatography-mass spectrometry |
| mCPBA | meta-Chloroperoxybenzoic acid |
| Me | Methyl |
| MeI | Methyl iodide |
| MeOH | Methanol |
| mg | Milligram |
| min | Minute(s) |
| mL | Milliliters |
| mmol | Millimoles |
| MsCl | Methanesulfonyl chloride |
| MTBE | tert-Butyl methyl ether |
| MW | Microwave |
| NMP | N-Methyl-2-pyrrolidone |
| NOE | Nuclear Overhauser effect |
| PdCl₂(dppf)•CH₂Cl₂ | [1,1′-bis(Diphenylphosphino)ferrocene]dichloropalladium(II), |
| | complex with dichloromethane |
| Pd(dppf)Cl₂ | (1,1′-Bis(diphenylphosphino)ferrocene)palladium(II) dichloride |
| Pd₂dba₃ | Tris(dibenzylideneacetone)dipalladium |
| PhMe | Toluene |
| PG | Protecting group |
| ROE | Rotating-frame Overhauser effect |
| RP | Reverse phase |
| RT | Room temperature |
| Rt | Retention time |
| SFC | Supercritical fluid chromatography |
| STAB | Sodium triacetoxyborohydride |
| T3P | Propanephosphonic acid anhydride |
| Tf₂O | Trifluoromethanesulfonic anhydride |
| TFA | Trifluoroacetic acid |
| TFAA | Trifluoroacetic anhydride |
| TFE | Trifluoroethanol |
| THF | Tetrahydrofuran |
| TPP | Triphenylphosphine |
| TPPO | Triphenylphosphine oxide |
| TLC | Thin layer chromatography |
| TMSCl | Trimethylsilyl chloride |
| pTsCl (TsCl) | 4-Toluenesulfonyl chloride |
| UHP | Urea hydrogen peroxide |
| UPLC | Ultra-performance liquid chromatography |

### LCMS

Instrument names: Shimadzu LC2020 Nexera Series; Shimadzu MS2020 N-Series; Agilent 1290 Infinity II series with 1.260 MSD.

Method A: Mobile Phase A. 10 mM NH₄HCO₃ in water, mobile phase B: ACN; Column: X Bridge C8 (50×4.6) mm, 3.5 μm.

Method B: Mobile Phase A: 0.1% FA in water, mobile phase B: 0.05% FA in ACN; Column: ZORBAX ECLIPSE PLUS C18 (50×2.1) mm, 1.8 μm.

Method C: Mobile Phase A: 0.1% TEA in water, mobile phase B: AC-N; Column: X Bridge C8 (50×4.6) mm, 3.5 μm.

Method D: Mobile Phase A: 10 mM NH₄HCO₃ in water, mobile phase B: ACN; Column: Phenomenex Kinetex EVO C18 (50×3.0) mm, 2.6 μm.

Method E: Mobile Phase A: 5 mM NH₄HCO₃ in water, mobile phase B: ACN; Column: Phenomenex Kinetex EVO C18 (50×3.0) mm, 2.6 μm.

Method F: Mobile Phase A: 0.1% FA in water, mobile phase B: 0.1% FA in ACN; Column: ZORBAX ECLIPSE PLUS C18 (50×2.1) mm, 1.8 μm.

Method G: Mobile Phase: A: 0.1% TFA in water, mobile phase B: ACN; Column: ZORBAX ECLIPSE PLUS C18 (50×2.1) mm, 1.8 μm.

### HPLC

Instrument names: Shimadzu LC; Prominence-I series instruments as followed using % with UV detection (Maxplot).

Method A: Mobile Phase A: 10 mM NH₄HCO₃ in water, mobile phase B: ACN; Flow Rate: 2.0 mL/min; Column: X-Bridge C8 (150×4.6) mm, 5 μm.

Method B: Mobile Phase A: 0.1% TFA in water, mobile phase B: ACN; Flow Rate: 1.0 mL/min; Column: Atlantis column C18 (250×4.6) mm, 5 μm.

Method C: Mobile Phase A: 10 mM NH₄OAc in water, mobile phase B: ACN; Flow Rate: 1.5 mL/min; Column: Gemini NX C18 (150×4.6) mm, 3 μm.

Method D: Mobile Phase A: 0.1% FA in water, mobile phase B: ACN; Flow Rate: 2.0 mL/min; Column: X-Select CSH C18 (150×4.6) mm, 5 μm.

Method E: Mobile Phase A: 0.1% TFA in water, mobile phase B: ACN: Flow Rate: 2.0 mL/min; Column: X-Select CSH C18 (150×4.6) mm, 5 μm.

Method F: Mobile Phase A: 10 mM NH₄OAc in water, mobile phase B: ACN; Flow Rate: 1.5 mL/min; Column: X-Bridge C8 (150×4.6) mm, 5 μm.

Method G: Mobile Phase A: 0.1% TFA in water, mobile phase B: ACN; Flow Rate: 1.2 mL/min; Column: Atlantis T3 C18 (150×4.6) mm, 3 μm.

Method H: Mobile Phase A: 10 mM NH₄HCO₃ in water, mobile phase B: ACN; Flow Rate: 1.2 mL/min; Column: X-Bridge C18 (150×4.6) mm, 3.5 μm.

Method I: Mobile Phase A: 0.1% FA in water, mobile phase B: ACN; Flow Rate: 1.2 mL/min; Column: Atlantis T3 CIS (150×4.6) mm, 3 μm.

### Prep HPLC

Instrument names: Agilent Technologies 1260 Infinity II Series LC/6125 MSD; Shimadzu LC 20 series Prep HPLC/MS2020.

Method A: Mobile Phase A: 10 mM NH₄HCO₃ in water; mobile phase B: ACN; Flow Rate: 15.0 mL/min; Column: Zorbax C18 (150×21.2) mm, 5 μm.

Method B: Mobile Phase A: 10 mM NH₄HCO₃ in water, mobile phase B: ACN; Flow Rate: 15.0 mL/min; Column: YMC Triart CIS (250×20) mm, 5 μm.

Method C: Mobile Phase A: 10 mM NH₄OAc in water; mobile phase B: ACN; Flow Rate: 15.0 mL/min; Column: Shimpack GIST Cis (150×19) mm, 5 μm.

Method D: Mobile Phase A: 10 mM NH₄HCO₃ in water; mobile phase B: ACN; Flow Rate: 15.0 mL/min; Column: X-Select CSH C18 (250×19) mm, 5 μm.

Method E: Mobile Phase A: 10 mM NH₄HCO₃ in water; mobile phase B: ACN; Flow Rate: 12.0 mL/min; Column: X-Select CSH C18 (250×19) mm, 5 μm.

Method F: Mobile Phase A: 10 mM NH₄HCO₃ in water; mobile phase B: ACN; Flow Rate: 15.0 mL/min; Column: X-Bridge C8 (150×19) mm, 5 μm.

Method G: Mobile phase A: 10 mM NH₄HCO₃ in water, mobile phase B: ACN; Flow Rate: 15.0 mL/min; Column: Phenomenex gemini-nx (250×21.2 mm) 5 μm.

Method H: Mobile Phase A: 10 mM NH₄HCO₃ in water; mobile phase B: ACN; Flow Rate: 10.0 mL/min; Column: YMC Triart C18 (250×20) mm, 5 μm.

Method I: Mobile Phase A: 10 mM NH₄HCO₃ in water, mobile phase B: ACN; Flow Rate: 10.0 mL/min; Column: X-Select CSH C18 (150×19) mm, 5 μm.

Method J: Mobile Phase A: 10 mM NH₄HCO₃ in water; mobile phase B: ACN; Flow Rate: 10.0 mL/min; Column: YMC C18 (250×20) mm, 5 μm.

Method K: Mobile Phase A: 10 mM NH₄HCO₃ in water; mobile phase B: ACN; Flow Rate: 10.0 mL/min; Column: X-Bridge C8 (150×19) mm, 5 μm.

Method L: Mobile Phase A: 10 mM NH₄HCO₃ in water; mobile phase B: ACN; Flow Rate: 15.0 mL/min; Column: Shimpack GIST C18 (150×19) mm, 5 μm.

Method M: Mobile Phase A: 10 mM NH₄HCO₃ in water; mobile phase B: ACN; Flow Rate: 10.0 m/min; Column: X-Bridge C8 (150×19) mm, 5 μm.

Method N: Mobile Phase A: 10 mM NH₄HCO₃ in water; mobile phase B: ACN; Flow Rate: 12.0 mL/min; Column: X-Bridge C8 (250×19) mm, 5 μm.

Method O: Mobile Phase A: 10 mM NH₄HCO₃ in water; mobile phase B: ACN; Flow Rate: 8.0 mL/min; Column: X-Bridge C8 (150×19) mm, 5 m.

Method P: Mobile Phase A: 10 mM NH₄HCO₃ in water, mobile phase B: ACN; Flow Rate: 15.0 mL/min; Column: Gemini C18 (250×21.2) mm, 5 μm.

Method Q: Mobile phase A: 10 mM NH₄HCO₃ in water, mobile phase B: ACN; Flow Rate: 13.0 mL/min; Column: Phenomenex gemini-nx (250×21.2 mm) 5 μm.

Method R: Mobile Phase A: 10 mM NH₄HCO₃ in water; mobile phase B: ACN; Flow Rate: 12.0 mL/min; Column: X-Select CSH CIS (150×19) mm, 5 μm.

Method S: Mobile Phase A: 10 mM NH₄OAc in water; mobile phase B: ACN; Flow Rate: 15.0 mL/min; Column: Shimpack C8 (150×20) mm, 5 μm.

Method T: Mobile Phase A: 0.1% Formic acid in water; mobile phase B: ACN; Flow Rate: 15.0 mL/min; Column: Zorbax C18 (250×21.2) mm, 5 μm.

### Chiral SFC

Instrument names: Shimadzu Nexera UC SFC Analytical; PIC—SFC Analytical-10

Method A: Mobile Phase A: CO₂; Co-solvent—0.5% Isopropylamine in MeOH (60:40); Flow Rate: 4.0 mL/min;% Co-Solvent: 40%; Column: LUX-C4 (250×4.6) mm, 5 sm.

Method B: Mobile Phase A: CO₂; Co-solvent—0.5% Isopropylamine in MeOH (60:40); Flow Rate: 4.0 mL/min; % Co-Solvent: 40%; Column: Whelk-(R,R) (250×4.6) mm, 5 μm.

Method C: Mobile Phase A: CO₂; Co-solvent—0.5% Isopropylamine in IPA (50:50); Flow Rate: 5.0 mL/min; % Co-Solvent: 50%; Column: LUX-A1 (250×4.6) mm, 5 μm.

Method D: Mobile Phase A: CO₂; Co-solvent—0.5% Isopropylamine in MeOH (60:40); Flow Rate: 3.0 mL/min; % Co-Solvent: 40%; Column: CHIRALPAK-IK (250×4.6) mm, 5 μm.

Method E: Mobile Phase A: CO₂; Co-solvent—0.5% Isopropylamine in MeOH (70:30); Flow Rate: 4.0 mL/min;% Co-Solvent: 30%; Column: LUX-A1 (250×4.6) mm, 5 μm.

Method F: Mobile Phase A: CO₂; Co-solvent—0.5% Isopropylamine in IPA (85:15); Flow Rate: 4.0 mL/min; % Co-Solvent: 15%; Column: LUX-A1 (250×4.6) mm, 5 μm.

Method G: Mobile Phase A: CO₂; Co-solvent—0.5% Isopropylamine in MeOH (60:40); Flow Rate: 4.0 mL/min; % Co-Solvent: 40%; Column: l-Cellulose B (250×4.6) mm, 5 μm.

Method H: Mobile Phase A: CO₂; Co-solvent—0.5% Isopropylamine in MeOH (90:10); Flow Rate: 4.0 mL/min;% Co-Solvent: 10%; Column: I-Cellulose B (250×4.6) mm, 5 μm.

Method I: Mobile Phase A: CO₂; Co-solvent—0.5% Isopropylamine in MeOH (65:35); Flow Rate: 4.0 mL/min; % Co-Solvent: 35%; Column: I-Cellulose B (250×4.6) mm, 5 μm.

Method J: Mobile Phase A: CO₂; Co-solvent—0.5% Isopropylamine in IPA (80:20); Flow Rate: 4.0 mL/min; % Co-Solvent: 20%; Column: 1-Cellulose B (250×4.6) mm, 5 μm.

Method K: Mobile Phase A: CO₂; Co-solvent—0.5% Isopropylamine in MeOH (60:40); Flow Rate: 4.0 mL/min;% Co-Solvent: 40%; Column: I-Cellulose Z (250×4.6) mm, 5 μm.

Method L: Mobile Phase A: CO₂; Co-solvent—0.5% Isopropylamine in IPA (70:30); Flow Rate: 5.0 mL/min; % Co-Solvent: 30%; Column: 1-Cellulose B (250×4.6) mm, 5 μm.

Method M: Mobile Phase A: CO₂; Co-solvent—0.5% Isopropylamine in MeOH (70:30); Flow Rate: 4.0 mL/min; % Co-Solvent: 30%; Column: CHIRALPAK-1K (250×4.6) mm, 5 μm.

Method N: Mobile Phase A: CO₂; Co-solvent—0.5% Isopropylamine in IPA (55:45); Flow Rate: 4.0 mL/min; % Co-Solvent: 45%; Column: Whelk-(R,R) (250×4.6) mm, 5 μm.

Method O: Mobile Phase A: CO₂; Co-solvent—0.5% Isopropylamine in MeOH (60:40); Flow Rate: 5.0 mL/min;% Co-Solvent: 40%; Column: I-Cellulose Z (250×4.6) mm, 5 μm.

Method P: Mobile Phase A: CO₂; Co-solvent—0.5% Isopropylamine in MeOH (70:30); Flow Rate: 4.0 mL/min;% Co-Solvent: 30%; Column: I-Cellulose Z (250×4.6) mm, 5 μm.

Method Q: Mobile Phase A: CO₂; Co-solvent—0.5% Isopropylamine in IPA (60:40); Flow Rate: 5.0 mL/min; % Co-Solvent: 40%; Column: I-Cellulose J (250×4.6) mm, 5 μm.

Method R: Mobile Phase A: CO₂; Co-solvent—0.5% Isopropylamine in IPA (50:50); Flow Rate: 5.0 mL/min; % Co-Solvent: 50%; Column: I-Cellulose C (250×4.6) mm, 5 μm.

Method S: Mobile Phase A: CO₂; Co-solvent—0.5% Isopropylamine in MeOH (75:25); Flow Rate: 4.0 mL/min;% Co-Solvent: 25%; Column: CHIRALPAK-IK (250×4.6) mm, 5 μm.

Method T: Mobile Phase A: CO₂; Co-solvent—0.5% Isopropylamine in MeOH (50:50); Flow Rate: 4.0 mL/min; % Co-Solvent: 50%; Column: I-Cellulose Z (250×4.6) mm, 5 μm.

Method U: Mobile Phase A: CO₂; Co-solvent—0.5% Isopropylamine in MeOH (65:35); Flow Rate: 4.0 mL/min;% Co-Solvent: 35%; Column: Whelk-(R,R) (250×4.6) mm, 5 μm.

Method V: Mobile Phase A: CO₂; Co-solvent—0.5% Isopropylamine in MeOH (60:40); Flow Rate: 4.0 mL/min; % Co-Solvent: 40%; Column: LUX-C2 (250×4.6) mm, 5 μm.

Method W: Mobile Phase A: CO₂; Co-solvent—0.5% Isopropylamine in MeOH (60:40); Flow Rate: 5.0 mL/min;% Co-Solvent: 40%; Column: Whelk-(R,R)(250×4.6) mm, 5 μm.

Method X: Mobile Phase A: CO₂; Co-solvent—0.5% Isopropylamine in MeOH (70:30); Flow Rate: 5.0 mL/min; % Co-Solvent: 30%; Column: 1-Cellulose C (250×4.6) mm. 5 μm.

Method Y: Mobile Phase A: CO₂; Co-solvent—0.5% Isopropylamine in MeOH (80:20); Flow Rate: 5.0 mL/min;% Co-Solvent: 20%; Column: CHIRALPAK-IK (250×4.6) mm, 5 μm.

Method Z: Mobile Phase A: CO₂; Co-solvent—0.5% Isopropylamine in IPA (70:30); Flow Rate: 5.0 mL/min; % Co-Solvent: 30%; Column: LUX-A1 (250×4.6) mm, 5 μm.

Method AA: Mobile Phase A: CO₂; Co-solvent—0.5% Isopropylamine in MeOH (80:20); Flow Rate: 4.0 mL/min;% Co-Solvent: 20%; Column: l-Cellulose Z (250×4.6) mm, 5 μm.

Method AB: Mobile Phase A: CO₂; Co-solvent—0.5% Isopropylamine in MeOH (70:30); Flow Rate: 5.0 mL/min; % Co-Solvent: 30%; Column: 1-Cellulose Z (250×4.6) mm, 5 μm.

Method AC: Mobile Phase A: CO₂; Co-solvent—0.5% Isopropylamine in IPA (70:30); Flow Rate: 4.0 mL/min;% Co-Solvent: 30% A; Column: I-Cellulose Z (250×4.6) mm, 5 μm.

Method AD: Mobile Phase A: CO₂; Co-solvent—0.5% Isopropylamine in MeOH (60:40); Flow Rate: 5.0 mL/min;% Co-Solvent: 40%; Column: LUX-C2 (250×4.6) mm, 5 μm.

Method AE: Mobile Phase A: CO₂; Co-solvent—0.5% Isopropylamine in MeOH (90:10); Flow Rate: 4.0 mL/min;% Co-Solvent: 10′%; Column: 1-Cellulose Z (250×4.6) mm, 5 μm.

Method AF: Mobile Phase A: CO₂; Co-solvent—0.5% Isopropylamine in IPA (55:45); Flow Rate: 5.0 mL/min; % Co-Solvent: 45%; Column: CHIRALPAK-1K (250×4.6) mm, 5 μm.

Method AG: Mobile Phase A: CO₂; Co-solvent—0.5% Isopropylamine in MeOH (60:40); Flow Rate: 4.0 mL/min; % Co-Solvent: 40%; Column: 1-Cellulose C (250×4.6) mm, 5 μm.

Method AH: Mobile Phase A: CO₂; Co-solvent—0.5% Isopropylamine in MeOH (60:40); Flow Rate: 5.0 mL/min;% Co-Solvent: 40%; Column: LUX-A3 (250×4.6) mm, 5 μm.

Method AI: Mobile Phase A: CO₂; Co-solvent—0.5% Isopropylamine in MeOH (92:8); Flow Rate: 4.0 mL/min; % Co-Solvent: 8%; Column: I-Cellulose B (250×4.6) mm, 5 μm.

Method AJ: Mobile Phase A: CO₂; Co-solvent—0.5% Isopropylamine in [ACN: MeOH (1:1)](55:45); Flow Rate: 4.0 mL/min; % Co-Solvent: 45%; Column: I-Cellulose Z (250×4.6) mm, 5 μm.

Method AK Mobile Phase A: CO₂; Co-solvent—0.5% Isopropylamine in MeOH (55:45); Flow Rate: 4.0 mL/min; % Co-Solvent: 45%; Column: CHIRALPAK-IK (250×4.6) mm, 5 μm.

Method AL Mobile Phase A: CO₂; Co-solvent—0.5% Isopropylamine in [ACN:MeOH (1:1)](55:45); Flow Rate: 5.0 mL/min; % Co-Solvent: 45%; Column: I-Cellulose C (250×4.6) mm, 5 μm.

Method AM: Mobile Phase A: CO₂; Co-solvent—0.5% Isopropylamine in MeOH (75:25); Flow Rate: 4.0 mL/min; % Co-Solvent: 25%; Column: I-Cellulose B (250×4.6) mm, 5 μm.

Method AN: Mobile Phase A: CO₂; Co-solvent—0.5% Isopropylamine in [ACN:MeOH (1:1)](50:50); Flow Rate: 5.0 mL/min; % Co-Solvent: 50%; Column: I-Cellulose C (250×4.6) mm, 5 μm.

### Chiral Prep SFC

Method A: Mobile Phase A: CO₂; Co-solvent—0.5% Isopropylamine in IPA (80:20); Flow Rate: 100 mL/min; % Co-Solvent: 20%; Column: 1-Cellulose B (250×21) mm, 5 μm.

Method B: Mobile Phase A: CO₂; Co-solvent—0.5% Isopropylamine in IPA (70:30); Flow Rate: 100 mL/min; % Co-Solvent: 30%; Column: I-Cellulose Z (250×30) mm, 5 μm.

Method C: Mobile Phase A: CO₂; Co-solvent—0.5% Isopropylamine in IPA (60:40); Flow Rate: 100 mL/min; % Co-Solvent: 40/%; Column: I-Cellulose Z (250×30) mm. 5 μm.

Method D: Mobile Phase A: CO₂; Co-solvent—0.5% Isopropylamine in IPA (90:10); Flow Rate: 100 m/min; % Co-Solvent: 10%; Column: I-Cellulose Z (250×30) mm, 5 μm.

Method E: Mobile Phase A: CO₂; Co-solvent—0.5% Isopropylamine in IPA (70:30); Flow Rate: 120 mL/min; % Co-Solvent: 30%; Column: I-Cellulose Z (250×30) mm, 5 μm.

Method F: Mobile Phase A: CO₂; Co-solvent—0.5% Isopropylamine in MeOH (90:10); Flow Rate: 100 mL/min; % Co-Solvent: 10%; Column: I-Cellulose B (250×30) mm, 5 μm.

Method G: Mobile Phase A: CO₂; Co-solvent—0.5% Isopropylamine in MeOH (90:10); Flow Rate: 50 mL/min; % Co-Solvent: 10%; Column: 1-Cellulose B (250×20) mm, 5 μm.

Method H: Mobile Phase A: CO₂; Co-solvent—0.5% Isopropylamine in MeOH (75:25); Flow Rate: 120 mL/min; % Co-Solvent: 25%; Column: Chiralpak IK (250×30) mm, 5 μm.

Method I: Mobile Phase A: CO₂; Co-solvent—0.5% Isopropylamine in IPA (70:30); Flow Rate: 100 mL/min; % Co-Solvent: 30%; Column: Lux A1 (250×30) mm, 5 μm.

### Achiral SFC

### Instrument Names: PIC-SFC-175; PIC—SFC-400

Method A: Mobile Phase A: CO₂; Co-solvent—0.5% Isopropylamine in IPA (70:30); Flow Rate: 5 mL/min; % Co-Solvent: 15%; Column: 4-Ethyl pyridine (250×4.6) mm, 5 μm.

### Achiral Prep SFC

### Instrument Names: PIC—SFC-175; PIC—SFC-400

Method A: Mobile Phase A: CO₂; Co-solvent—0.5% Isopropylamine in MeOH (85:15); Flow Rate: 100 mL/min; % Co-Solvent: 15%; Column: 4-Ethyl pyridine (250×30) mm, 5 μm.

Method B: Mobile Phase A: CO₂; Co-solvent—MeOH (70:30); Flow Rate: 100 mL/min; % Co-Solvent: 30%; Column: 4-Ethyl pyridine (250×30) mm, 5 μm.

### Intermediates

### Intermediate 1. Methyl 5-fluoro-2-(pyrimidin-5-yloxy)benzoate

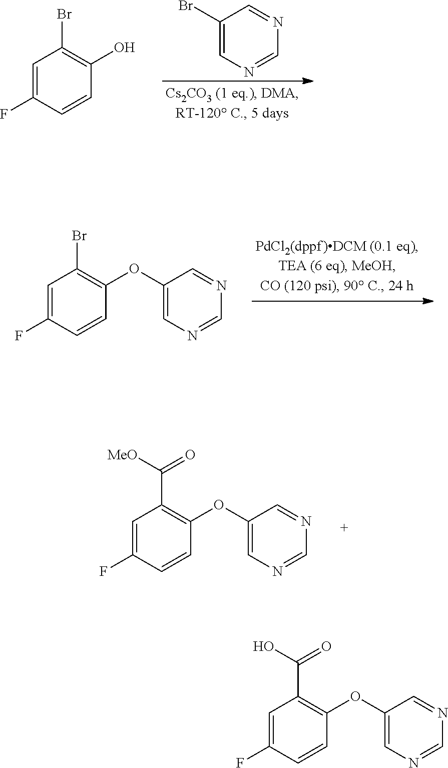 [SMILES: Oc1ccc(F)cc1Br ; Brc1cncnc1 ; Fc1ccc(Oc2cncnc2)c(Br)c1 ; COC(=O)c1cc(F)ccc1Oc1cncnc1 ; O=C(O)c1cc(F)ccc1Oc1cncnc1]

### Step 1. 5-(2-Bromo-4-fluorophenoxy)pyrimidine

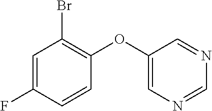 [SMILES: Fc1ccc(Oc2cncnc2)c(Br)c1]

Step 1 was performed following the same procedure in two batches, a 450 g batch and a 200 g batch.

In a dried, 10 L four neck round bottom flask under a nitrogen atmosphere, 2-bromo-4-fluorophenol (450 g, 2356 mmol) was dissolved in DMA (2.5 L) at RT. Cs₂CO₃ (768 g, 2356 mmol) and 5-bromopyrimidine (375 g, 2356 mmol) were then added at RT, and the reaction was stirred at 120° C. for 5 days. The reaction progress was monitored by LCMS and TLC (10% EtOAc in hexane). The reaction mixtures from both batches (450 g and 200 g) were combined, quenched with water (3 L), and extracted with MTBE (2×5 L). The combined organic layer was washed with water (2 L) and brine solution (2 L), dried over anhydrous sodium sulfate and filtered, and the filtrate was concentrated under reduced pressure. The crude product was purified by silica-gel column chromatography using EtOAc in hexane (product eluted at 16% EtOAc in hexane) to obtain 5-(2-bromo-4-fluorophenoxy)pyrimidine (380 g, 38.9% combined yield) as an oil. ¹H NMR (400 MHz, DMSO-d₆): δ 9.00 (s, 1H), 8.57 (s, 2H), 7.81 (dd, J=2.9, 8.2 Hz, 1H), 7.46-7.33 (m, 2H); LCMS (Method F): Rt=1.79 min, m/z=269.2 [M+H]⁺, 78.45%.

### Step 2. Methyl 5-fluoro-2-(pyrimidin-5-yloxy)benzoate and 5-fluoro-2-(pyrimidin-5-yloxy)benzoic acid

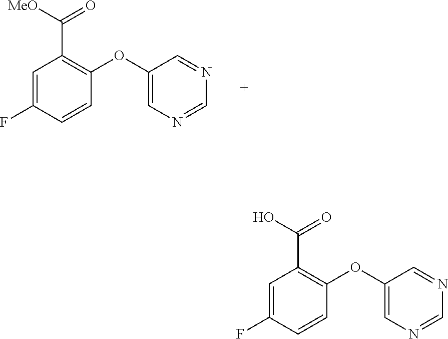 [SMILES: COC(=O)c1cc(F)ccc1Oc1cncnc1 ; O=C(O)c1cc(F)ccc1Oc1cncnc1]

Step 2 was performed following the same procedure in sixteen batches, a 21.5 g batch, and 15×20 g batches.

In a 300 mL stainless steel mini clave, 5-(2-bromo-4-fluorophenoxy)pyrimidine (20 g, 74.3 mmol) was dissolved in MeOH (150 mL), and the solution was degassed with argon for 30 min. TEA (61.0 mL, 446 mmol) was added, and the reaction was degassed for another 5 min. To the above solution, PdCl₂(dppf)-CH₂Cl₂ adduct (6.07 g, 7.43 mmol) was added, and the reaction was stirred under 120 psi carbon monoxide gas atmosphere (Caution: toxic gas) at 90° C. for 24 h. The reaction was monitored by TLC (40% EtOAc in hexane). After completion, the reaction was filtered through a Celite® pad, and the pad was washed with methanol (150 mL). The filtrate was concentrated under reduced pressure to obtain crude product (22 g). Similarly, all sixteen batches (21.5 g batch and 15×20 g batches) were filtered, and the filtrate was concentrated under reduced pressure to obtain crude product separately. The combined crude material (320 g) from sixteen batches was collected and dissolved in EtOAc (2 L). To this solution, 10% sodium bicarbonate (2 L) solution was added, and the mixture was extracted with EtOAc (2×2 L). The combined organic layer was dried over sodium sulfate and filtered, and the filtrate was concentrated under reduced pressure. The crude product was purified by silica gel flash column chromatography using EtOAc in hexane as the eluent (product eluted at 15-20% EtOAc in hexane) to obtain methyl 5-fluoro-2-(pyrimidin-5-yloxy)benzoate (198 g, 62.2% combined yield) as a solid. Further, the aqueous layer was acidified with conc. HCl (pH ~2), and the precipitate was filtered and dried under vacuum to obtain crude 5-fluoro-2-(pyrimidin-5-yloxy)benzoic acid (25 g, 7.99% yield) as a solid. This material was used without further purification.

Methyl 5-fluoro-2-(pyrimidin-5-yloxy)benzoate: ¹H NMR (400 MHz, DMSO-d₆): δ 8.95 (s, 1H), 8.50 (s, 2H), 7.74 (dd, J=3.1, 8.8 Hz, 1H), 7.60 (ddd, J=3.3, 7.9, 9.0 Hz, 1H), 7.44 (dd, J=4.6, 9.0 Hz, 1H), 3.74 (s, 3H); LCMS (Method D): Rt=1.65 min, m/z=249.1 [M+H]⁺, 92.80%.

5-Fluoro-2-(pyrimidin-5-yloxy)benzoic acid: ¹H NMR (400 MHz, DMSO-d): δ 13.24 (br s, 1H), 8.92 (s, 1H), 8.47 (s, 2H), 7.70 (dd, J=3.3, 8.9 Hz, 1H), 7.55 (ddd, J=3.3, 7.9, 8.9 Hz, 1H), 7.40 (dd, 0.1=4.6, 9.0 Hz, 1H); LCMS (Method F): Rt=1.26 min, m/z=235.2 [M+H]⁺, 74.16%.

### Intermediate 2. 5-Fluoro-2-(pyrimidin-5-yloxy)benzoic acid

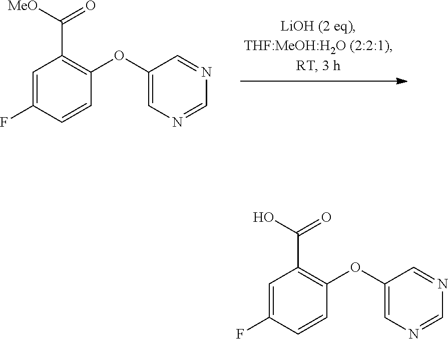 [SMILES: COC(=O)c1cc(F)ccc1Oc1cncnc1 ; O=C(O)c1cc(F)ccc1Oc1cncnc1]

In a 1000 mL 3 neck round bottom flask under nitrogen atmosphere, methyl 5-fluoro-2-(pyrimidin-5-yloxy)benzoate (32 g, 129 mmol) in 2:2:1 MeOH:THF:H₂O (125 mL:125 mL:62.5 mL), LiOH·H₂O (10.82 g, 258 mmol) was added at RT. The reaction was stirred at RT for 3 h, and monitored by TLC (10% MeOH in DCM). After completion, the reaction was concentrated under reduced pressure, and the aqueous layer was acidified with 1.5 N HC (pH~6). The solid was filtered, and the solid was transferred to a round bottom flask and co-distilled with toluene under reduced pressure to obtain crude 5-fluoro-2-(pyrimidin-5-yloxy)benzoic acid (29.485 g, 93% yield) as a solid. This material was used without further purification. ¹H NMR (400 MHz, DMSO-d₆): δ 13.42 (br s, 1H), 8.92 (s, 1H), 8.47 (s, 2H), 7.70 (dd, J=3.2, 8.8 Hz, 1H), 7.56 (ddd, J=3.3, 7.9, 9.0 Hz, 1H), 7.40 (dd, J=4.6, 9.0 Hz, 1H); LCMS (Method B): Rt=1.66 min, m/z=235 [M+H]⁺; 95.07%.

### Intermediate 3. N-Ethyl-5-fluoro-2-hydroxy-N-isopropylbenzamide

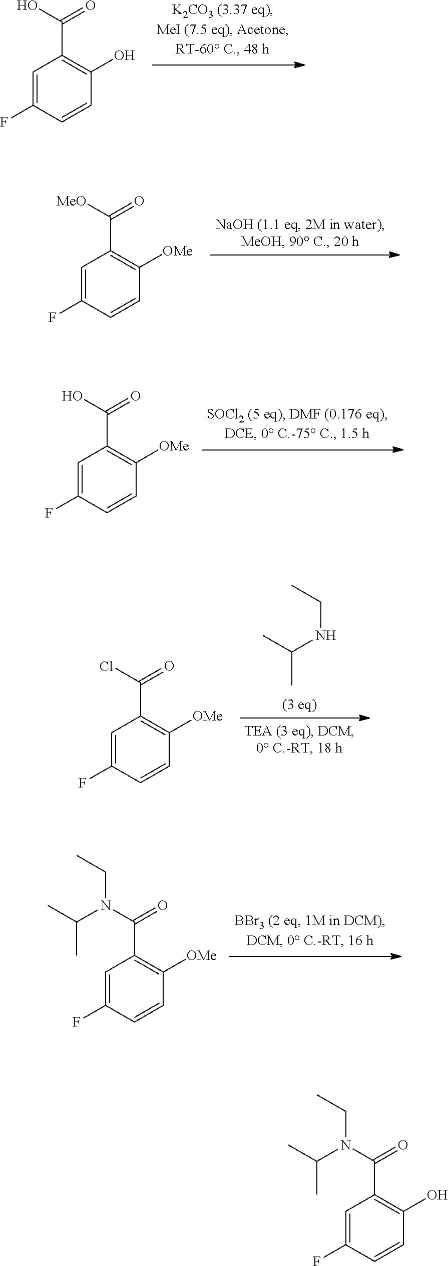

### Step 1. Methyl 5-fluoro-2-methoxybenzoate

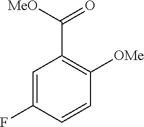 [SMILES: COC(=O)c1cc(F)ccc1OC]

In a dried, 25 L four neck round bottom flask under a nitrogen atmosphere, 5-fluoro-2-hydroxybenzoic acid (800 g, 5125 mmol) was dissolved in acetone (7 L). K₂CO₃ (2387 g, 17271 mmol) was added, followed by dropwise addition of Mel (2394 mL, 38437 mmol) at RT. The resulting reaction was heated at 60° C. for 48 h. The reaction progress was monitored by TLC (50% EtOAc in hexane). The reaction was filtered and concentrated under reduced pressure. The residue was diluted with water (4000 mL) and extracted with EtOAc (3×2000 mL). The combined organic layer was dried over anhydrous sodium sulfate and filtered, and the filtrate was concentrated under reduced pressure to obtain methyl 5-fluoro-2-methoxybenzoate (930 g, 97% yield) as a gummy liquid. ¹H NMR (400 MHz, DMSO-d₆): δ 7.47-7.37 (m, 2H), 7.18 (dd, J=4.4, 9.1 Hz, 1H), 3.81 (s, 3H), 3.80 (s, 3H); LCMS (Method F): Rt=1.70 min, m/z=185.2 [M+H]⁺; HPLC (Method A): Rt=5.05 min, 98.07%.

### Step 2. 5-Fluoro-2-methoxybenzoic acid

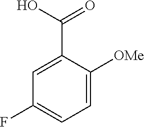 [SMILES: COc1ccc(F)cc1C(=O)O]

In a 25 L four-neck round bottom flask, methyl 5-fluoro-2-methoxybenzoate (1.45 kg, 7.87 mol) was dissolved in MeOH (5 L). To this solution, NaOH (4330 mL, 8.66 mol, 2 M in H₂O) was added at RT, and the reaction was stirred at 90° C. for 20 h. The reaction progress was monitored by TLC (50% EtOAc in hexane). The reaction was concentrated, diluted with water (5 L), and acidified to pH 2 with 1.5 N HCl (5 L). The resulting solid compound was filtered, washed with water (5 L), and dried under reduced pressure to obtain 5-fluoro-2-methoxybenzoic acid (1.2 kg, 89% yield) as a solid. ¹H NMR (400 MHz, DMSO-d₆): δ 12.85 (br s, H), 7.41 (dd, J=3.3, 8.8 Hz, 1H), 7.35 (ddd, J=3.3, 8.1, 9.1 Hz, 1H), 7.14 (dd, J=4.3, 9.1 Hz, 1H), 3.80 (s, 3H); LCMS (Method F): Rt=1.31 min, m/z=169.2 [M−H]⁻, 99.65%.

### Step 3. S-Fluoro-2-methoxybenzoyl chloride

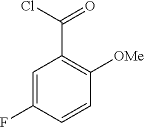 [SMILES: COc1ccc(F)cc1C(=O)Cl]

To a stirred solution of 5-fluoro-2-methoxybenzoic acid (12.5 g, 73.5 mmol)) in DCE (125 mL), thionyl chloride (26.6 mL, 367 mmol) was added slowly at 0° C., then DMF (1 mL, 12.91 mmol) was added slowly at 0° C. under a nitrogen atmosphere. The reaction was stirred at 0° C. for 10 min, then stirred at 75° C. for 1.5 h. The reaction progress was monitored by TLC (30% EtOAc in hexane. The reaction was quenched with MeOH to check the reaction progress). After completion, the reaction was concentrated under reduced pressure to obtain crude 5-fluoro-2-methoxybenzoyl chloride (12.5 g, 90% yield). This material was used without further purification.

### Step 4. N-Ethyl-5-fluoro-N-isopropyl-2-methoxybenzamide

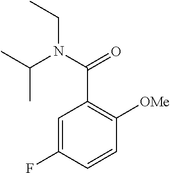 [SMILES: CCN(C(=O)c1cc(F)ccc1OC)C(C)C]

To a solution of 5-fluoro-2-methoxybenzoyl chloride (50 g, 265 mmol) in DCM (500 mL) under a nitrogen atmosphere, TEA (112 mL, 795 mmol) and N-ethylpropan-2-amine (96 mL, 795 mmol) were added at 0° C., and the reaction was stirred at RT for 18 h. The reaction progress was monitored by TLC (20% EtOAc in hexane). After completion, the reaction was quenched with water (150 mL) and the mixture was extracted with DCM (2×250 mL). The combined organic layer was washed with water (150 mL) and brine solution (100 mL). The organic layer was dried over anhydrous sodium sulfate and filtered, and the filtrate was concentrated under reduced pressure. The crude was purified by silica gel flash column chromatography using EtOAc in hexane (product eluted at 20% EtOAc in hexane) to obtain N-ethyl-5-fluoro-N-isopropyl-2-methoxybenzamide (60 g, 92% yield) as a liquid. ¹H NMR (400 MHz, DMSO-d₆): δ 7.23-7.16 (m, 1H), 7.11-7.05 (m, 1H), 7.05-7.01 (m, 1H), 3.76 (s, 3H), 3.56 (spt, J=6.7 Hz, 1H), 3.32-3.23 (m, 2H), 1.25-0.87 (m, 9H); LCMS (Method D): Rt=1.71 min, m/z=240.1 [M+H]⁺, 97.20%.

### Step 5. N-Ethyl-5-fluoro-2-hydroxy-N-isopopylbenzamide

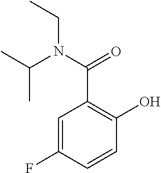 [SMILES: CCN(C(=O)c1cc(F)ccc1O)C(C)C]

In a dried, 1000 mL three-neck round bottom flask under a nitrogen atmosphere, N-ethyl-5-fluoro-N-isopropyl-2-methoxybenzamide (25 g, 104 mmol) was dissolved in DCM (250 mL). To this solution, BBr₃ (209 ml, 209 mmol, 1 M in DCM) was added at 0° C. and the reaction was stirred at RT for 16 h. The reaction progress was monitored by TLC (10% MeOH in DCM). After completion, the reaction was quenched with ice-cold NaHCO₃ solution (100 mL) and the mixture was extracted with EtOAc (3×100 mL). The combined organic layer was dried over anhydrous sodium sulfate and filtered, and the filtrate was concentrated under reduced pressure to obtain crude N-ethyl-5-fluoro-2-hydroxy-N-isopropylbenzamide (23.5 g, 97% yield) as a liquid. This material was used without further purification. LCMS (Method D): Rt=1.51 min, nm/z=226.0 [M+H]⁺, 97.14%.

### Intermediate 4. Methyl 2-((4-chloropyrimidin-5-yl)oxy)-5-fluorobenzoate

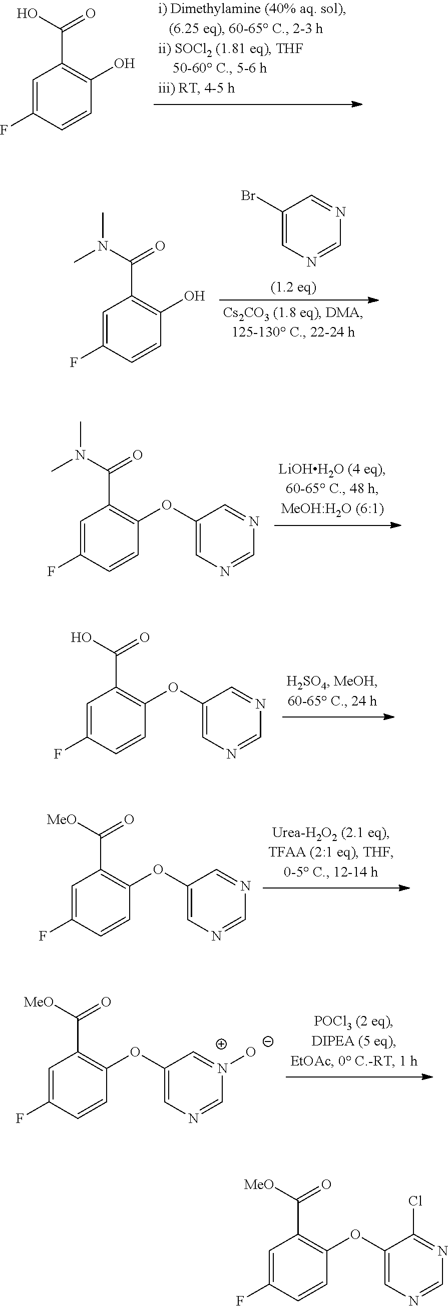

### Step 1. 5-Fluoro-2-hydroxy-N,N-dimethylbenzamide

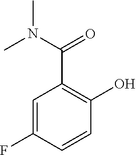

A 40% aqueous dimethylamine solution (140.0 L, 1200.0 mol) was heated at 60-65° C. for 2-3 h to release dimethylamine gas, which was purged into a cooled solution (0-5° C.) of THF (250.0 L).

In another reactor, thionyl chloride (41.0 kg, 347.4 moles) was added to a solution of 5-fluorosalicylic acid (30.0 kg, 192.0 moles) in THF (100.0 L) over a period of 1-2 h. The reaction mixture was heated to 50-60° C. for 5-6 h. After completion of the reaction, as judged by TLC, the reaction mixture was concentrated completely, THF was added and distilled out to remove the remaining thionyl chloride. The acid chloride thus obtained was dissolved in THF (30.0 L) and was added to the dimethylamine-THF solution at 0-5° C. The reaction mixture was stirred at RT for 4-5 h. After completion of the reaction, the reaction mixture was concentrated under a vacuum below 40° C. Ice water was added, and the mixture was stirred for 1-2 h at room temperature to precipitate a solid. The solid was filtered and washed with water. The solid was triturated with cyclohexane and centrifuged to give 5-fluoro-2-hydroxy-N,N-dimethylbenzamide (28.5 kg, 81% yield) as a solid. ¹H NMR (400 MHz, DMSO-d₆): δ2.81 (s, 3H), 2.94 (s, 3H), 6.83-6.87 (m, 1H), 6.92-6.95 (m, 1H), 7.02-7.07 (m, 1H), 9.76 (br, 1H); HPLC (% purity): 99.63%.

### Step 2. 5-Fluoro-N,N-dimethyl-2-(pyrimidin-5-yloxy)benzamide

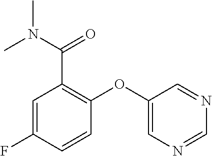 [SMILES: CN(C)C(=O)c1cc(F)ccc1Oc1cncnc1]

To a solution of 5-fluoro-2-hydroxy-N,N-dimethylbenzamide (28.0 kg, 153.0 moles) and 5-bromopyrimidine (29.16 kg, 183.0 moles) in dimethyl acetamide (180.0 L) was added cesium carbonate (89.73, 270.0 moles). The reaction mixture was heated to 125-130° C. for 22-24 h. After complete consumption of the starting material, as judged by TLC, the reaction mixture was cooled to 35-40° C. and filtered to remove the cesium carbonate. The reaction mixture was concentrated under vacuum. The residue thus obtained was dissolved in water (84.0 L) and extracted with DCM (2×56.0 L). The DCM layer was washed with 10% aqueous sodium hydroxide solution, followed by dilute HCl solution, dried over sodium sulfate, filtered, and concentrated to give 5-fluoro-N,N-dimethyl-2-(pyrimidin-5-yloxy)benzamide (24.0 kg, 60.1% yield) as a semisolid. ¹H NMR (400 MHz, DMSO-d₆): δ8.94 (s, 1H), 8.54 (s, 2H), 7.25-7.34 (m, 3H), 2.85 (s, 3H), 2.84 (s, 3H); HPLC (% purity): 86.75%.

### Step 3. 5 Fluoro-2-(pyrimidin-5-yloxy)benzoic acid

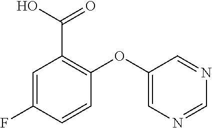 [SMILES: O=C(O)c1cc(F)ccc1Oc1cncnc1]

To a solution of 5-fluoro-N,N,-dimethyl-2-(pyrimidin-5-yloxy)benzamide (23.5 kg, 90.03 moles) in 6:1 MeOH/H₂O (140.0 L) was added LiOH·H₂O (15.5 kg, 360.0 moles), and the reaction was heated at 60-65° C. for 48 h. The reaction mixture was cooled to 40° C., and the methanol was distilled out in vacuum below 40° C. The residue thus obtained was dissolved in water. The aqueous layer was washed with ethyl acetate. The aqueous layer was treated with activated carbon and filtered on Hyflo. The aqueous layer was acidified with concentrated HCl (1.5 L) to pH~1.0-2.0 to precipitate a solid. This solid was filtered, washed with water, and dried at 40-45° C. to give 5-fluoro-2-(pyrimidin-5-yloxy)benzoic acid (13.5 kg, 64.1% yield) as a solid. ¹H NMR (400 MHz, DMSO-d₆): δ 13.40 (br s, 1H), 8.92 (s, 1H), 8.46 (s, 2H), 7.69-7.71 (m, 1H), 7.52-7.57 (m, 1H), 7.38-7.42 (m, 1H); HPLC (% purity): 96.94%.

### Step 4. Methyl 5-fluoro-2-(pyrimidin-S-yloxy)benzoate

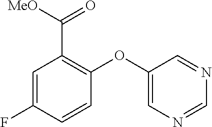 [SMILES: COC(=O)c1cc(F)ccc1Oc1cncnc1]

To a cooled solution of 5-fluoro-2-(pyrimidin-5-yloxy)benzoic acid (13.5 kg, 57.69 moles) in methanol (90.0 L) was added sulfuric acid (2.16 L), and the reaction was heated at 60-65° C. for 24 h. The reaction mixture was cooled to room temperature, and the methanol was distilled out completely under reduced pressure. The residue thus obtained was dissolved in DCM (54.0 L).

The DCM layer was washed with 5% sodium bicarbonate solution and saturated sodium chloride solution. The organic layer was dried over anhydrous sodium sulfate, filtered, and concentrated to give methyl 5-fluoro-2-(pyrimidin-5-yloxy)benzoate (9.6 kg, 67.1% yield) as a solid. ¹H NMR (400 MHz, DMSO-d₆: δ 8.94 (s, 1H), 8.50 (s, 2H), 7.72-7.75 (m, 1H), 7.57-7.62 (m, 1H), 7.41-7.45 (m, 1H), 3.73 (s, 3H); HPLC (% purity): 98.37%; assay by Q-NMR (%): 99.21%.

### Step 5. 5-(4-Fluoro-2-(methoxycarbonyl)phenoxy)pyrimidine 1-oxide

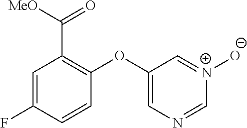 [SMILES: COC(=O)c1cc(F)ccc1Oc1cnc[n+]([O-])c1]

To a cooled solution of methyl 5-fluoro-2-(pyrimidin-5-yloxy)benzoate (9.6 kg, 38.7 moles) in THF (150 L) was added UHP (7.64 kg, 81.2 moles) at 0-5° C. (over a period of 30 min), and the reaction was stirred for 10 min at 0-5° C. TFAA (17.0 kg, 81.0 moles) was then added dropwise over 30-40 min at 0-5° C. The reaction was stirred at 0-5° C. for 12-14 h. After complete consumption of starting material, as judged by TLC, the reaction mixture was quenched by the addition of 5% NaHCO₃ solution at 0-5° C. DCM (60.0 L) was then added at 5-10° C., and the mixture was stirred for 20 min. The aqueous and organic layers were separated. The organic layer was washed with 5% NaHCO₃. Aqueous sodium thiosulfate solution was then added to the organic layer (DCM layer), and the mixture was stirred for 12 hours at 10° C. The organic layer was separated, dried over anhydrous Na₂SO₄, filtered, and concentrated under reduced pressure to give 5-(4-fluoro-2-(methoxycarbonyl)phenoxy)pyrimidine-1-oxide (5.12 kg, 50.0% yield) as solid. ¹HNMR (400 MHz, DMSO-d₆): δ8.86 (s, 1H), 8.44 (s, 1H), 8.01 (s, 1H), 7.72-7.75 (m, 1H), 7.59-7.64 (m, 1H), 7.49-7.52 (m, 1H), 3.76 (s, 3H); HPLC (% purity): 99.60%.

### Step 6. Methyl 2-((4-chloropyrimidin-5-yl)oxy)-5-fluorobenzoate

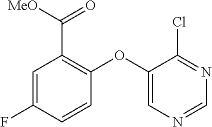 [SMILES: COC(=O)c1cc(F)ccc1Oc1cncnc1Cl]

In a 100 mL two neck round bottom flask under a nitrogen atmosphere, 5-(4-fluoro-2-(methoxycarbonyl)phenoxy)pyrimidine 1-oxide (20 g, 76 mmol) was dissolved in EtOAc (200 mL). To this solution, DIPEA (65.3 mL, 378 mmol) was added at 0° C., and the reaction was stirred at the same temperature for 15 min. To this reaction mixture, POCl₃ (14.15 mL, 151 mmol) was added dropwise at 0° C. The reaction was warmed to RT and stirred for 1 h. The reaction was monitored by TLC (50% EtOAc in hexane). The reaction was concentrated under reduced pressure to afford crude compound. The crude compound was purified by silica gel flash column chromatography using EtOAc in hexane (product eluted at 25% EtOAc in hexane) to obtain methyl 2-((4-chloropyrimidin-5-yl)oxy)-5-fluorobenzoate (16.5 g, 72.2% yield) as a solid. ¹HNMR (400 MHz, DMSO-d₆): δ 8.81 (s, 1H), 8.30 (s, 1H), 7.76 (dd, J=3.1, 8.8 Hz, 1H), 7.61 (ddd, J=3.3, 7.9, 9.0 Hz, 1H), 7.46 (dd, J=4.6, 9.0 Hz, 1H), 3.75 (s, 3H); LCMS (Method E): Rt=1.74 min, m/z=283.1 [M+H]⁺; 93.65%.

### Intermediate 5. 2-((4-Chloropyrimidin-5-yl)oxy)-N-ethyl-5-fluoro-N-isopropylbenzamide

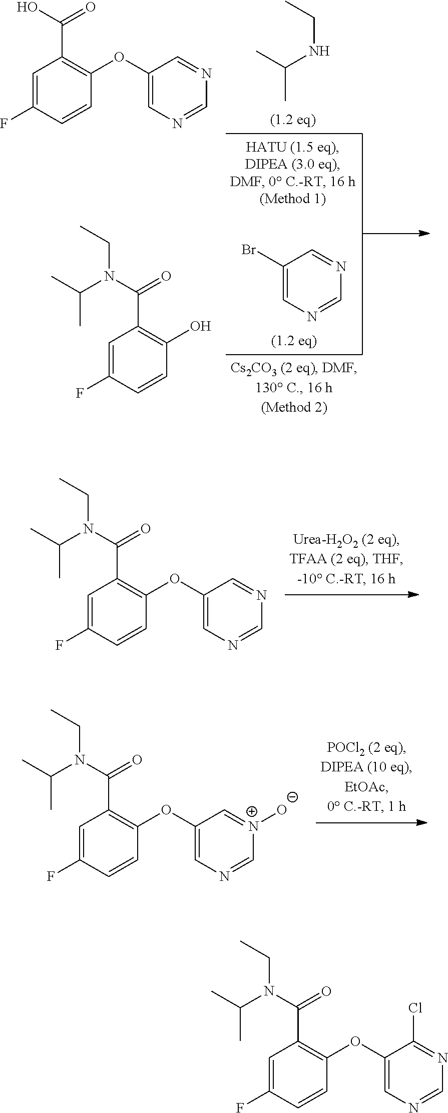

### Step 1. N-Ethyl-5-fluoro-N-isopropyl-2-(pyrimidin-5-yloxy)benzamide (Method 1)

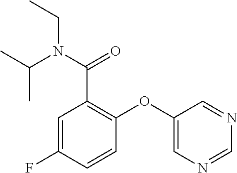 [SMILES: CCN(C(=O)c1cc(F)ccc1Oc1cncnc1)C(C)C]

In a dried, 500 mL two neck round bottom flask under a nitrogen atmosphere, 5-fluoro-2-(pyrimidin-5-yloxy)benzoic acid (29.485 g, 126 mmol) was dissolved in DMF (300 mL). To this solution, DIPEA (66.0 mL, 378 mmol), and HATU (71.8 g, 189 mmol) were added at 0° C., followed by N-ethylpropan-2-amine (18.29 mL, 151 mmol) at RT, and the reaction was stirred at RT for 16 h. The reaction was monitored by TLC (10%. MeOH in DCM). The reaction was diluted with water (500 mL) and extracted with EtOAc (3×200 mL). The combined organic layer was washed with cold water (500 mL) and brine solution (3×200 mL), dried over anhydrous sodium sulfate, and filtered. The filtrate was concentrated under reduced pressure. The crude product was purified by silica gel flash column chromatography using EtOAc in hexane (product eluted at 87% EtOAc in hexane) to obtain N-ethyl-5-fluoro-N-isopropyl-2-(pyrimidin-5-yloxy)benzamide (41.2 g, 84% yield) as a sticky solid. ¹H NMR (400 MHz, DMSO-d₆): δ 8.95 (s, 1H), 8.56-8.45 (m, 2H), 7.40-7.30 (m, 3H), 3.74 (spt, J=6.7 Hz, 1H), 3.45-3.33 (m, 1H), 3.23-3.10 (m, 1H), 1.16-1.02 (6H), 1.03-0.96 (m, 3H); LCMS (Method B): Rt=2.26 min, m/z=304.3 [M+H]⁺, 77.68%.

### Step 1. N-Ethyl-5-fluoro-N-isopropyl-2-(pyrimidin-5-yloxy)benzamide (Method 2)

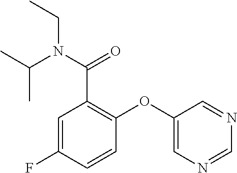 [SMILES: CCN(C(=O)c1cc(F)ccc1Oc1cncnc1)C(C)C]

In a dried, 1 L three neck round bottom flask under a nitrogen atmosphere, N-ethyl-5-fluoro-2-hydroxy-N-isopropylbenzamide (19 g, 84 mmol) was dissolved in DMF (200 mL). To this reaction mixture, Cs₂CO₃ (55.0 g, 169 mmol) and 5-bromopyrimidine (16.09 g, 101 mmol) were added at RT, and the reaction was stirred at 130° C. for 16 h. The reaction progress was monitored by TLC (50% EtOAc in hexane). After completion, the reaction was quenched with ice-cold water (200 mL) and extracted with EtOAc (3×300 mL). The combined organic layer was dried over anhydrous sodium sulfate and filtered, and the filtrate was concentrated under reduced pressure. The crude product was purified by silica gel flash column chromatography using EtOAc in hexane (product eluted at 70% EtOAc in hexane) to obtain N-ethyl-5-fluoro-isopropyl-2-(pyrimidin-5-yloxy)benzamide (17.1 g, 56.1% yield) as a liquid. ¹H NMR (400 MHz, DMSO-d₆): δ 8.95 (s, 1H), 8.53-8.50 (m, 2H), 7.38-7.32 (m, 3H), 3.73 (spt,)=6.6 Hz, 1H), 3.43-3.34 (m, 1H), 3.21-3.11 (m, 1H), 1.26-1.02 (m, 6H), 1.02-0.85 (m, 3H); LCMS (Method D); Rt=1.58 min, m/z=304.1 [M+H]⁺; 83.91%.

### Step 2. 5-(2-(Ethyl isopropyl) carbamoyl)-4-fluorophenoxy)pyrimidine 1-oxide

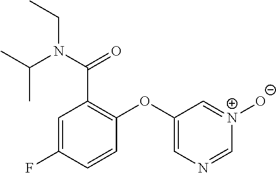 [SMILES: CCN(C(=O)c1cc(F)ccc1Oc1cnc[n+]([O-])c1)C(C)C]

Step 2 was performed following the same procedure in two batches, 2×20.7 g batches.

In a dried, 500 mL three neck round bottom flask under nitrogen atmosphere, N-ethyl-S-fluoro-N-isopropyl-2-(pyrimidin-5-yloxy)benzamide (20.7 g, 68.2 mmol) was dissolved in THE (200 mL). To this solution, urea hydrogen peroxide (12.84 g, 136 mmol) and TFAA (19.28 mL, 136 mmol)) were added slowly at −10° C. The reaction was stirred at RT for 16 h. The reaction was monitored by TLC (100% EtOAc). The reaction mixture from both batches (2×20.7 g) was combined, cooled to 0° C., quenched with aqueous sodium bicarbonate solution (200 mL), and extracted with EtOAc (2×250 mL). The combined organic layer was washed with aqueous sodium thiosulfate solution (500 mL), dried over sodium sulfate, and filtered. The filtrate was concentrated under reduced pressure to afford crude 5-(2-(ethyl(isopropyl)carbamoyl)-4-fluorophenoxy)pyrimidine 1-oxide (46.15 g, 86.02% combined yield) as a gummy liquid. This material was used without further purification. LCMS (Method B): Rt=1.87 min, m/z=320.0 [M+H]⁺, 57.84%.

### Step 3. 2-((4-Chloropyrimidin-5-yl)oxy)-N-ethyl-5-fluoro-N-isopropylbenzamide

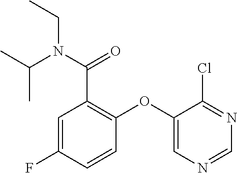 [SMILES: CCN(C(=O)c1cc(F)ccc1Oc1cncnc1Cl)C(C)C]

In a dried, 500 mL two neck round bottom flask under nitrogen atmosphere, 5-(2-(ethyl(isopropyl)carbamoyl)-4-fluorophenoxy)pyrimidine 1-oxide (10 g, 31.3 mmol) was dissolved in EtOAc (100 mL). To this solution, DIPEA (54.7 mL, 313 mmol) was added at 0° C., then phosphoryl trichloride (5.86 mL, 62.6 mmol) was added dropwise at 0° C. The reaction was stirred at RT for 1 h. The reaction progress was monitored by TLC (100% EtOAc). The reaction was concentrated under reduced pressure. The crude product was purified by silica gel flash column chromatography using EtOAc in hexane (product eluted at 58% EtOAc in hexane) to obtain 2-((4-chloropyrimidin-5-yl)oxy)-N-ethyl-5-fluoro-N-isopropylbenzamide (3.049 g, 27.3% yield) as a sticky liquid. ¹H NMR (400 MHz, DMSO-d₆): δ 8.83-8.76 (m, 1H), 8.34-8.27 (m, 1H), 7.43-7.33 (m, 3H), 3.72 (spt, J=6.6 Hz, 1H), 3.42-3.34 (m, 1H), 3.24-3.12 (m, 1H), 1.18-1.07 (m, 6H), 1.05-0.98 (m, 3H); LCMS (Method B): Rt=2.36 min, m/z 338.0 [M+H]⁺, 94.54%.

### Intermediate 6. 2-((4-Chloropyrimidin-5yl)oxy)-5-fluoro-N,N-diisopropylbenzamide

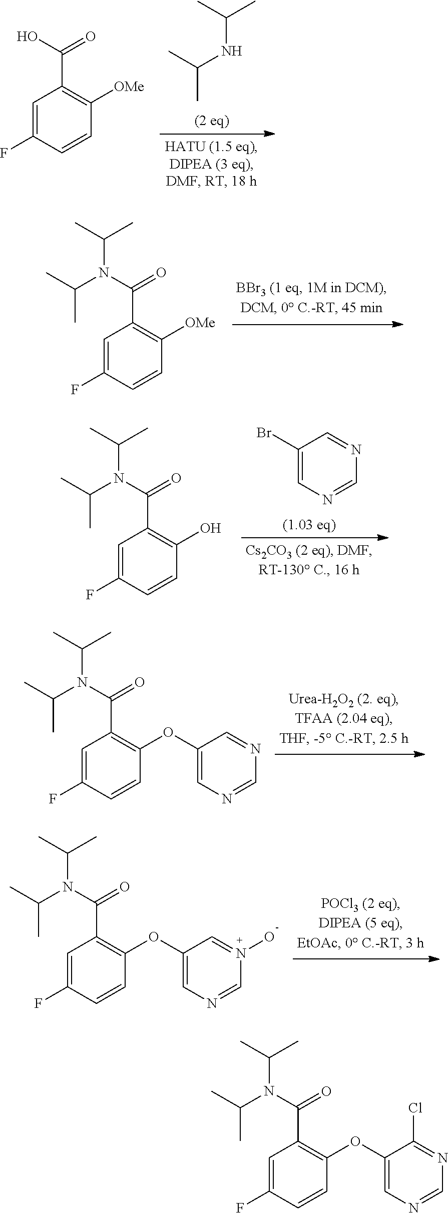

### Step 1. 5-Fluoro-N,N-diisopropyl-2-methoxybenzamide

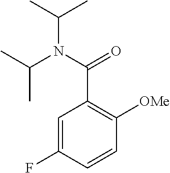 [SMILES: COc1ccc(F)cc1C(=O)N(C(C)C)C(C)C]

In a dried, 2 L four neck round bottom flask under a nitrogen atmosphere, 5-fluoro-2-methoxybenzoic acid (46 g, 270 mmol) was dissolved in DMF (460 mL). To this solution, diisopropylamine (77 mL, 541 mmol), DIPEA (142 mL, 811 mmol), and HATU (154 g, 406 mmol) were added at 0° C. The reaction was stirred at RT for 18 h, and monitored by TLC (50% EtOAc in hexane). After completion, the reaction was quenched with ice-cold water (2 L) and extracted with 10% EtOAc in hexane (2×2 L). The combined organic layer was dried over anhydrous sodium sulfate and filtered. The filtrate was concentrated under reduced pressure to obtain crude 5-fluoro-N,N-diisopropyl-2-methoxybenzamide (66 g, 94% yield) as a solid. This material was used without further purification. ¹H NMR (400 MHz, DMSO-d₆): δ 7.21-7.12 (m, 1H), 7.09-7.03 (m, 1H), 6.98 (dd, J=3.1, 8.3 Hz, 1H), 3.76 (s, 3H), 3.60-3.44 (m, 2H), 1.42 (t, J=6.8 Hz, 6H), 1.10 (d, J=6.6 Hz, 3H), 1.01 (d, J=6.8 Hz, 3H); LCMS (Method F): Rt=2.00 min, m/z=254.2 [M+H]⁺; HPLC (Method A): Rt=6.08 min, 97.06%.

### Step 2. 5-Fluoro-2-hydroxy-N,N-diisopropylbenzamide

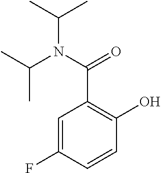 [SMILES: CC(C)N(C(=O)c1cc(F)ccc1O)C(C)C]

To a stirred solution of 5-fluoro-N,N-diisopropyl-2-methoxybenzamide (25 g, 99 mmol) in DCM (500 mL), BBr₃ (99 mL, 99 mmol, IM in DCM) in DCM was added slowly at 0° C. under a nitrogen atmosphere. The reaction was allowed to warm slowly to RT and stirred there for 45 min.

The progress of the reaction was monitored by TLC (40% EtOAc in hexane). After completion, the reaction was cooled to 0° C., quenched with ice-cold water (400 mL), and extracted with DCM (3×200 mL). The combined organic layer was washed with brine solution (200 mL), dried over anhydrous sodium sulfate and filtered. The filtrate was concentrated under reduced pressure to obtain crude 5-fluoro-2-hydroxy-N,N-diisopropylbenzamide (23.7 g, 100% yield). This material was used without further purification. ¹H NMR (400 MHz, DMSO-d₆): δ 9.14-8.42 (m, 1H), 7.06-6.99 (m, 1H), 6.99-6.94 (m, 1H), 6.88 (dd, J=3.0, 8.6 Hz, 1H), 1.41 (d, J=6.8 Hz, 12H); LCMS (Method F): Rt=1.77 min, m/z=240.4 [M+H]⁺, 99.72%. This material was used without further purification.

### Step 3. S-Fluoro-N,N-diisopropyl-2-(pyrimidin-S-yloxy)benzamide

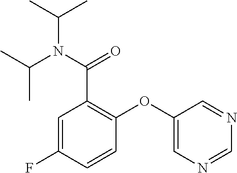 [SMILES: CC(C)N(C(=O)c1cc(F)ccc1Oc1cncnc1)C(C)C]

In a dried, 1 L four neck round bottom flask under a nitrogen atmosphere, 5-fluoro-2-hydroxy-N,N-diisopropylbenzamide (25 g, 104 mmol) was dissolved in DMF (250 mL). Cs₂CO₃ (68.1 g, 209 mmol) and 5-bromopyrimidine (17.11 g, 108 mmol) were then added at RT, and the reaction was stirred at 130° C. for 16 h. The reaction progress was monitored by TLC (40/EtOAc in hexane). After completion, the reaction was quenched with ice-cold water (400 mL) and extracted with EtOAc (3×400 mL). The combined organic layer was dried over anhydrous sodium sulfate and filtered, and the filtrate was concentrated under reduced pressure. The crude was purified by silica gel flash column chromatography using EtOAc in hexane (product eluted at 24% EtOAc in hexane) to obtain 5-fluoro-N,N-diisopropyl-2-(pyrimidin-5-yloxy)benzamide (16.3 g, 46.9% yield) as a solid. ¹H NMR (400 MHz, DMSO-d₆): δ 8.94 (s, 1H), 8.51 (s, 2H), 7.39-7.28 (m, 3H), 3.67 (spt, J=6.6 Hz, 1H), 3.57-3.43 (m, 1H), 1.39 (d, J=6.8 Hz, 3H), 1.17 (d, J=6.8 Hz, 3H), 1.09 (d, J=6.6 Hz, 3H), 1.04 (d, J=6.6 Hz, 3H); LCMS (Method F): Rt=1.82 min, m/z=318.4 [M+H]⁺, 95.35%.

### Step 4. S-(2-(Diisopropylcarbamoyl)-4-fluorophenoxy)pyrimidine 1-oxide

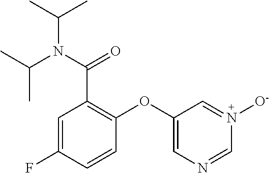

To a stirred solution of 5-fluoro-N,N-diisopropyl-2-(pyrimidin-5-yloxy)benzamide (16 g, 50.4 mmol) in THF (170 mL), urea hydrogen peroxide (9.49 g, 101 mmol) was added, and then, TFAA (14.53 mL, 103 mmol) was added dropwise over 30 min at −5° C. The reaction was allowed to stir at RT for 2 h, and monitored by TLC (30% EtOAc in hexane). After completion, the reaction was quenched with saturated sodium thiosulfate solution (100 mL) and then saturated ammonium carbonate solution (100 mL). The mixture was extracted with EtOAc (2×200 mL), and the organic layer was washed with brine solution (100 mL). The combined organic layer was dried over anhydrous sodium sulfate and filtered, and the filtrate was concentrated under reduced pressure. The residue was washed with n-heptane (2×100 mL) to obtain crude 5-(2-(diisopropylcarbamoyl)-4-fluorophenoxy)pyrimidine 1-oxide (16 g, 80% yield) as a solid. LCMS (Method A): Rt=1.76 min, m/z=334.3 [M+H]⁺; HPLC (Method A): Rt=4.82 min, 79.44%. This material was used without further purification.

### Step 5. 2-((4-Chloropyrimidin-5-yl)oxy)-5-fluoro-N,N-diisopropylbenzamide

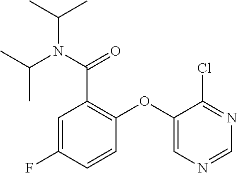 [SMILES: CC(C)N(C(=O)c1cc(F)ccc1Oc1cncnc1Cl)C(C)C]

In a dried, 500 mL two neck round bottom flask under a nitrogen atmosphere, 5-(2-(diisopropylcarbamoyl)-4-fluorophenoxy)pyrimidine 1-oxide (16 g, 48.0 mmol) was dissolved in EtOAc (160 mL). To this solution, DIPEA (41.8 mL, 240 mmol) was added at 0° C., then POCl₃ (8.97 mL, 96 mmol) was added dropwise at 0° C., and the reaction was stirred at the same temperature for 30 min. The reaction was allowed to stir at RT for 2.5 h. The reaction progress was monitored by TLC (30% EtOAc in hexane). The reaction was concentrated under reduced pressure. The crude was purified by silica gel flash column chromatography using EtOAc in hexane (product eluted at 30% EtOAc in hexane) to obtain 2-((4-chloropyrimidin-5-yl)oxy)-5-fluoro-N,N-diisopropylbenzamide (9 g, 51.2% yield) as a solid). ¹H NMR (400 MHz, DMSO-d₆): δ 8.73 (s, 1H), 8.23 (s, 1H), 7.16-7.09 (m, 1H), 7.09-7.00 (m, 2H), 3.78 (spt, J=6.7 Hz, 1H), 3.51 (spt, J=6.8 Hz, 1H), 1.51 (d, J=6.8 Hz, 3H), 1.36 (d, J=6.9 Hz, 3H), 1.28 (d, J=6.8 Hz, 3H), 1.16 (d, J=6.6 Hz, 3H); LCMS (Method F): Rt=2.08 min, m/z=352.4 [M+H]⁺, 96.76%.

### Intermediate 7. tert-Butyl (4aS,8aS)-hexahydro-2H-pyrido[4,3-b][1,4]oxazine-6(5H)-carboxylate

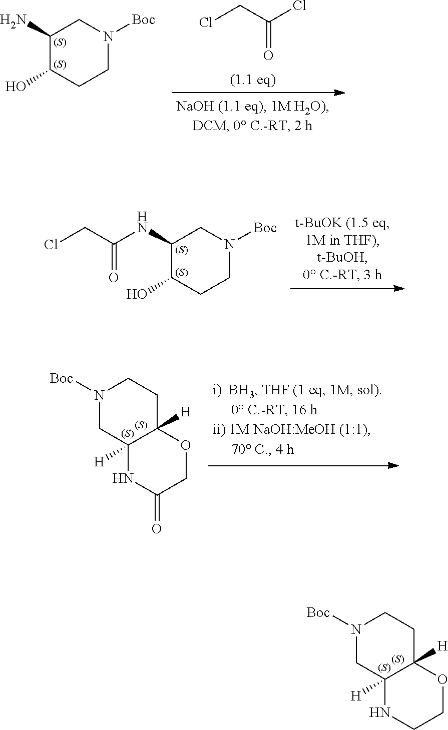

### Step 1. tert-Butyl (3S,4S)-3-(2-chloroacetamido)-4-hydroxypiperidine-1-carboxylate

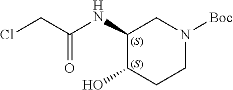

To a stirred solution of tert-butyl (3S,4S)-3-amino-4-hydroxypiperidine-1-carboxylate (10 g, 46.2 mmol) in DCM 20 mL, IM NaOH (50.9 mL, 50.9 mmol) was added at 0° C. To this reaction mixture, a solution of 2-chloroacetyl chloride (4.05 mL, 50.9 mmol) in DCM (10 mL) was added slowly, and the reaction was stirred at RT for 2 h. The reaction progress was monitored by TLC (10% MeOH in DCM). After completion, the reaction was quenched with water (50 mL) and extracted with DCM (3×100 mL). The combined organic layer was washed with brine (100 mL), dried over anhydrous sodium sulfate and filtered. The filtrate was concentrated under reduced pressure to obtain crude tert-butyl (3S,4S)-3-(2-chloroacetamido)-4-hydroxypiperidine-1-carboxylate (13.0 g, 95% yield) as a semi-solid. This material was used without further purification. ¹H NMR (400 MHz, CDCl₃): δ 6.84 (br s, 1H), 4.08 (s, 21), 3.96-3.89 (m, 1H), 3.85-3.74 (m, 2H), 3.74-3.65 (m, 1H), 3.32-3.18 (m, 2H), 1.98-1.88 (m, 1H), 1.67-1.56 (m, 1H), 1.50-1.47 (s, 9H), one proton was merged with solvent peaks; LCMS (Method D): Rt=1.37 min, m/z=291.2 [M−H]⁻, 87.53%.

### Step 2. tert-Butyl (4aS,8aS)-3-oxohexahydro-2H-pyrido[4,3-b][1,4]oxazine-6(5H)-carboxylate

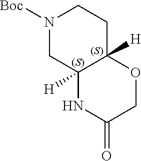

In a 500 mL two neck round bottom flask under argon atmosphere, tert-butyl (3S,4S)-3-(2-chloroacetamido)-4-hydroxypiperidine-1-carboxylate (13 g, 44.4 mmol) was dissolved in t-BuOH (130 mL). To this solution, potassium tert-butoxide (66.6 mL, 66.6 mmol; 1M in THF) was added at 0° C., and the reaction was stirred at RT for 3 h. The reaction progress was monitored by TLC (100% EtOAc). After completion, the reaction was cooled to 0° C., quenched with water (80 mL), and extracted with EtOAc (3×100 mL). The combined organic layer was washed with brine (100 mL), dried over anhydrous sodium sulfate and filtered, and the filtrate was concentrated under reduced pressure. The crude was purified by silica gel flash column chromatography using EtOAc in hexane (product eluted at 100% EtOAc) to obtain tert-butyl (4aS,8aS)-3-oxohexahydro-2H-pyrido[4,3-b][1,4]oxazine-6(5H)-carboxylate (7.0 g, 61.1% yield) as a solid. ¹H NMR (400 MHz, DMSO-d₆): δ8.23 (br s, 1H), 4.18-3.99 (m, 4H), 3.40-3.34 (m, 2H), 3.03-2.95 (m, 1H), 2.90-2.69 (m, 1H), 1.89-1.81 (m, 1H), 1.42 (m, 9H), 1.38-1.32 (m, 1H); LCMS (Method C): Rt=1.64 min, m/z=255.1 [M−H]⁻, 97.83%. Chiral SFC (Method 1): Rt=1.46 min, 100%.

### Step 3. tert-Butyl (4aS,8aS)-hexahydro-2H-pyrido[4,3-b][1,4]oxazine-6(5H)-carboxylate

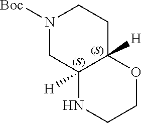

To a stirred solution of tert-butyl (4aS,8aS)-3-oxohexahydro-2H-pyrido[4,3-b][1,4]oxazine-6(5H)-carboxylate (7.0 g, 27.3 mmol) in THF (70 mL), borane tetrahydrofuran complex (54.6 mL, 54.6 mmol; 1 M in THF) was added at 0° C. under a nitrogen atmosphere. The reaction was stirred at RT for 16 h. The reaction progress was monitored by TLC (10% MeOH in DCM). After completion, the reaction was cooled to 0° C. and quenched with methanol (70 mL) and IM NaOH (70 mL). The reaction was stirred at 70° C. for 4 h. The reaction was cooled to RT and the mixture was concentrated under reduced pressure. Water (50 mL) was added to the residue and the mixture was extracted with EtOAc (3×100 mL). The combined organic layer was washed with brine (100 mL), dried over anhydrous sodium sulfate and filtered, and the filtrate was concentrated under reduced pressure. The crude was purified by silica gel flash column chromatography using methanol in DCM (product eluted at 5% MeOH in DCM) to obtain tert-butyl (4aS,8aS)-hexahydro-2H-pyrido[4,3-b][1,4]oxazine-6(5H)-carboxylate (3.0 g, 44.9% yield) as a semi-solid. ¹H NMR (400 MHz, DMSO-d₆): δ 4.04-3.82 (m, 2H), 3.75-3.69 (m, 1H), 3.52-3.44 (m, 1H), 3.09-3.00 (m, 1H), 2.83-2.68 (m, 3H), 2.47-2.30 (m, 1H), 2.25-2.16 (m, 1H), 1.71-1.64 (m, 1H), 1.42-1.37 (s, 9H), 1.35-1.22 (m, 1H), one proton merged with solvent peaks; LCMS (Method B): Rt=1.46 min, m/z=243.3 [M+H]⁺, 99.76%. Chiral SFC (Method F): Rt=1.83 min, 80.16%.

### Intermediate 8. tert-Butyl (4aR,8aR)-hexahydro-2H-pyrido[4,3-b][1,4]oxazine-6(5H)-carboxylate

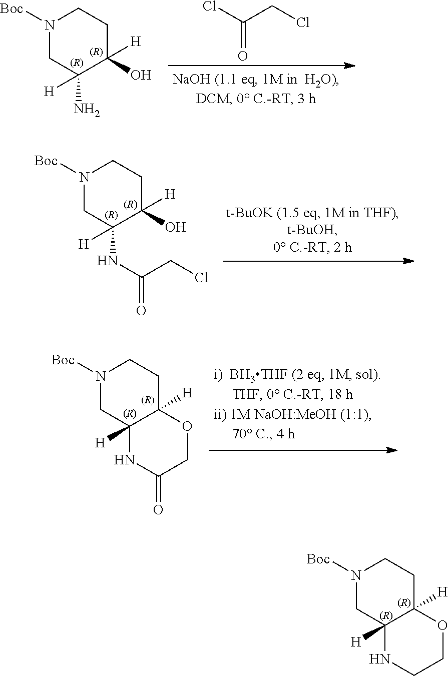

### Step 1. tert-Butyl (3R,4R)-3-(2-chloroacetamido)-4-hydroxypiperidine-1-carboxylate

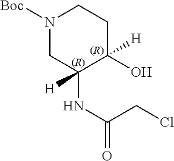

This compound was synthesized following the procedure described for the synthesis of Intermediate 7, step 1, starting with 1 equivalent tert-butyl (3R,4R)-3-amino-4-hydroxypiperidine-1-carboxylate and 1 equivalent of chloroacetyl chloride. After work-up, the organic layer was dried over sodium sulfate and filtered, and the filtrate was concentrated under reduced pressure to afford crude tert-butyl (3R,4R)-3-(2-chloroacetamido)-4-hydroxypiperidine-1-carboxylate as a sticky solid. This material was used without further purification.

Yield: 94%; ¹H NMR (400 MHz, CDCl₃): δ 6.81 (br s, 1H), 4.09 (s, 2H), 3.97-3.90 (m, 1H), 3.86-3.76 (m, 2H), 3.76-3.67 ((m, 1H), 3.34-3.16 (m, 2H), 1.90-1.89 (m, 1H), 1.68-1.55 (m, 1H), 1.49 (s, 9H), one proton was merged with solvent peaks; LCMS (Method D): Rt=1.31 min, m/z=291.1 [M−H]⁻, 98.27%.

### Step 2. tert-Butyl (4aR,8aR)-3-oxohexahydro-2H-pyrido[4,3-b][1,4]oxazine-6(5H)-carboxylate

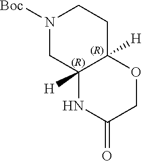

This compound was synthesized following the procedure described for the synthesis of Intermediate 7, step 2, starting with 1 equivalent tert-butyl (3R,4R)-3-(2-chloroacetamido)-4-hydroxypiperidine-1-carboxylate. After work-up, the crude was purified by silica gel flash column chromatography using EtOAc in hexane (product eluted at 80% to 100% EtOAc in hexane) to obtain tert-butyl (4aR,8aR)-3-oxohexahydro-2H-pyrido[4,3-b][1,4]oxazine-6(5H)-carboxylate as a sticky liquid.

Yield: 63.9%; LCMS-ELSD (Method D): Rt=1.44 min, m/z=255.1 [M−H]⁻, 99.88%.

### Step 3. tert-Butyl (4aR,8aR)-hexahydro-2H-pyrido[4,3-b][1,4]oxazine-6(5H)-carboxylate

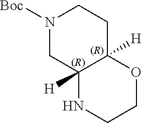

This compound was synthesized following the procedure described for the synthesis of Intermediate 7, step 3, starting with 1 equivalent tert-butyl (4aR,8aR)-3-oxohexahydro-2H-pyrido[4,3-b][1,4]oxazine-6(5H)-carboxylate, except that 2 equivalents of BH₃·THF (1M in THF) was used. After work-up, the crude was purified by silica gel flash column chromatography using MeOH in DCM (product eluted at 5% MeOH in DCM) to afford tert-butyl (4aR,8aR)-hexahydro-2H-pyrido[4,3-b][1,4]oxazine-6(5H)-carboxylate as a gummy solid.

Yield: 74%; ¹H NMR (400 MHz, DMSO-d₆): δ 4.03-3.84 (m, 2H), 3.75-3.69 (m, 1H), 3.52-3.44 (m, 1H), 3.10-3.01 (m, 1H), 2.84-2.65 (m, 3H), 2.47-2.35 (m, 1H), 2.25-2.17 (m, 1H), 1.74-1.64 (m, 1H), 1.41-1.38 (m, 9H), 1.36-1.22 (m, 1H), one proton merged with solvent peaks.

### Intermediate 9. (±)-tert-Butyl (trans)-octahydro-6H-pyrido[3,4-b][1,4]oxazine-6-carboxylate

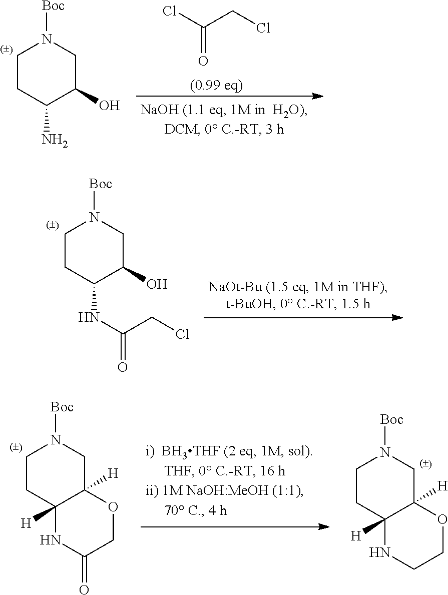 [SMILES: CC(C)(C)OC(=O)N1CC[C@@H](N)[C@H](O)C1 ; O=C(Cl)CCl ; CC(C)(C)OC(=O)N1CC[C@@H](NC(=O)CCl)[C@H](O)C1 ; CC(C)(C)OC(=O)N1CC[C@H]2NC(=O)CO[C@@H]2C1 ; CC(C)(C)OC(=O)N1CC[C@H]2NCCO[C@@H]2C1]

### Step 1. (+)-tert-Butyl (trans)-4-(2-chloroacetamido)-3-hydroxypiperidine-1-carboxylate

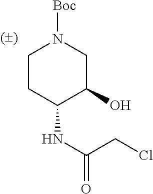 [SMILES: CC(C)(C)OC(=O)N1CC[C@@H](NC(=O)CCl)[C@H](O)C1]

This compound was synthesized following the procedure described for the synthesis of Intermediate 7, step 1, starting with 1 equivalent (±)-tert-butyl (trans)-4-amino-3-hydroxypiperidine-1-carboxylate and 0.99 equivalent of chloroacetyl chloride. After completion, the reaction was quenched with water and the mixture was extracted with EtOAc. The combined organic layer was washed with brine, dried over anhydrous sodium sulfate and filtered. The filtrate was concentrated under reduced pressure to obtain crude (±)-tert-butyl (trans)-4-(2-chloroacetamido)-3-hydroxypiperidine-1-carboxylate as a liquid. This material was used without further purification.

Yield: 59%; LCMS (Method F): Rt=1.46 min, m/z=193.2 [M+H−100]⁺, 89.22%.

### Step 2. (±)-tert-Butyl (trans)-2-oxooctahydro-6H-pyrido[3,4-b][1,4]oxazine-6-carboxylate

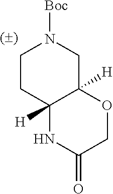 [SMILES: CC(C)(C)OC(=O)N1CC[C@H]2NC(=O)CO[C@@H]2C1]

This compound was synthesized following the procedure described for the synthesis of Intermediate 7, step 2, starting with 1 equivalent (±)-tert-butyl (trans)-4-(2-chloroacetamido)-3-hydroxypiperidine-1-carboxylate. After work-up, the crude was purified by silica gel flash column chromatography using EtOAc in hexane (product eluted at 80% to 100/% EtOAc in hexane) to obtain (±)-tert-butyl (trans)-2-oxooctahydro-6H-pyrido[3,4-b][1,4]oxazine-6-carboxylate as a solid.

Yield: 36%; LCMS (Method F): Rt=1.49 min, m/z=257.3 [M+H]⁺, 97.36%.

### Step 3. (±)-tert-butyl (trans)-octahydro-6H-pyrido[3,4-b][1,4]oxazine-6-carboxylate

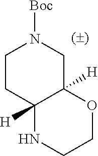 [SMILES: CC(C)(C)OC(=O)N1CC[C@H]2NCCO[C@@H]2C1]

This compound was synthesized following the procedure described for the synthesis of Intermediate 7, step 3, starting with 1 equivalent (±)-tert-butyl (trans)-2-oxooctahydro-6H-pyrido[3,4-b][1,4]oxazine-6-carboxylate, except that 2 equivalents of BH₃·THF (1M in THF) was used. After work-up, the crude was purified by silica gel flash column chromatography using MeOH in DCM (product eluted at 5% MeOH in DCM) to afford (±)-tert-butyl (trans)-octahydro-6H-pyrido[3,4-b][1,4]oxazine-6-carboxylate as a liquid.

Yield: 65.1%; LCMS (Method F): Rt=1.11 min, m/z=243.4 [M+H]⁺, 84.60%.

### Intermediate 10. (2-Azaspiro[3.3]heptan-6-yl)methanol hydrochloride

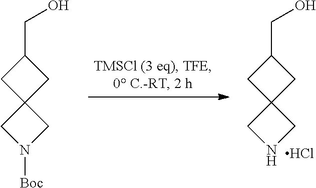 [SMILES: CC(C)(C)OC(=O)N1CC2(CC(CO)C2)C1 ; OCC1CC2(CNC2)C1 ; Cl]

To a stirred solution of tert-butyl 6-(hydroxymethyl)-2-azaspiro[3.3]heptane-2-carboxylate (23 g, 101 mmol) in TFE (230 mL), TMSCl (25.9 mL, 202 mmol) was added dropwise at 0° C. The reaction was stirred at RT for 1.5 h, and monitored by TLC (100% EtOAc). After completion, the reaction was concentrated under reduced pressure. The residue was reconcentrated from EtOAc (2×100 mL), then triturated with EtOAc (2×100 mL). The organic layer was decanted and the solid was dried under vacuum to obtain crude (2-azaspiro[3.3]heptan-6-yl)methanol hydrochloride (14.6 g, 88% yield) as a solid. This material was used without further purification. LCMS-ELSD (Method B): Rt=0.31 min, m/z=128.2 [M+H]⁺, 99.62%.

### Intermediate 11. N-Ethyl-5-fluoro-2-((4-(6-(hydroxymethyl)-2-azaspiro[3.3]heptan-2-yl)pyrimidin-5-yl)oxy)-N-isopropylbenzamide

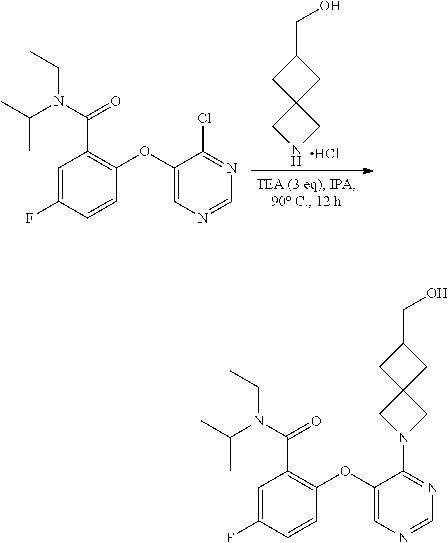 [SMILES: CCN(C(=O)c1cc(F)ccc1Oc1cncnc1Cl)C(C)C ; OCC1CC2(CNC2)C1 ; Cl ; CCN(C(=O)c1cc(F)ccc1Oc1cncnc1N1CC2(CC(CO)C2)C1)C(C)C]

To a solution of (2-azaspiro[3.3]heptan-6-yl)methanol hydrochloride (0.240 g, 1.467 mmol) in 2-propanol (5 mL), DIPEA (0.768 mL, 4.40 mmol) and 2-((4-chloropyrimidin-5-yl)oxy)-N-ethyl-5-fluoro-N-isopropylbenzamide (0.495 g, 1.467 mmol) were added at 0° C. The reaction was stirred at 90° C. for 12 h, and monitored by TLC (100% EtOAc). After completion, the reaction was diluted with water (30 mL) and the mixture was extracted with EtOAc (3×30 mL). The combined organic layer was washed with brine (20 mL), dried over anhydrous sodium sulfate and filtered, and the filtrate was concentrated under reduced pressure. The crude product (300 mg) was purified by silica gel flash column chromatography using EtOAc in hexane (product eluted at 100% EtOAc) to afford to obtain N-ethyl-5-fluoro-2-((4-(6-(hydroxymethyl)-2-azaspiro[3.3]heptan-2-yl)pyrimidin-5-yl)oxy)-N-isopropylbenzamide (240 mg, 38.2% yield) as a semi-solid. LCMS (Method D): Rt=1.50 min, m/z=429.1 [M+H]⁺, 96.14%.

### Intermediate 12. N-Ethyl-5-fluoro-2-((4-(6-formyl-2,6-diazaspiro[3.3]heptan-2-yl)pyrimidin-5-yl)oxy)-N-isopropylbenzamide

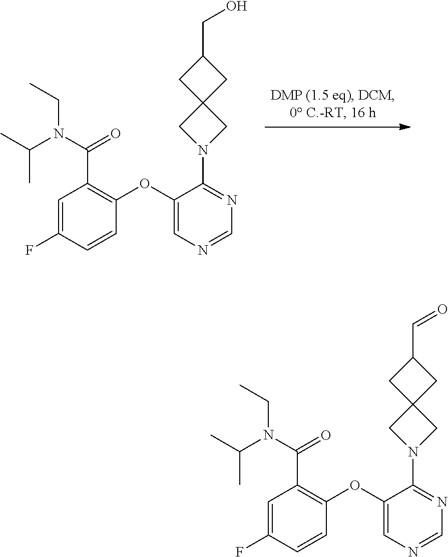 [SMILES: CCN(C(=O)c1cc(F)ccc1Oc1cncnc1N1CC2(CC(CO)C2)C1)C(C)C ; CCN(C(=O)c1cc(F)ccc1Oc1cncnc1N1CC2(CC(C=O)C2)C1)C(C)C]

In a two neck round bottom flask under a nitrogen atmosphere, N-ethyl-5-fluoro-2-((4-(6-(hydroxymethyl)-2-azaspiro[3.3]heptan-2-yl)pyrimidin-5-yl)oxy)-N-isopropylbenzamide (365 mg, 0.852 mmol) was dissolved in DCM (5 mL). To this solution, DMP (542 mg, 1.278 mmol) was added at 0° C., and the reaction was stirred at RT for 16 h. The reaction progress was monitored by TLC (100% EtOAc). After completion, the reaction was quenched with sodium bicarbonate solution (20 mL), and the mixture was extracted with DCM (2×50 mL). The combined organic layer was washed with sodium bicarbonate solution (3×20 mL), dried over anhydrous sodium sulfate and filtered. The filtrate was concentrated under reduced pressure to afford crude N-ethyl-5-fluoro-2-((4-(6-formyl-2-azaspiro[3.3]heptan-2-yl)pyrimidin-5-yl)oxy)-N-isopropylbenzamide (250 mg, 38.3% yield) as a liquid. This material was used without further purification. LCMS (Method B): Rt=1.36 min, m/z=427.2 [M+H]⁺, 55.63%.

### Intermediate 13. (2-(5-(2-(Ethyl(isopropyl)carbamoyl)-4-fluorophenoxy)pyrimidin-4-yl)-2-azaspiro[3.3]heptan-6-yl)methyl methanesulfonate

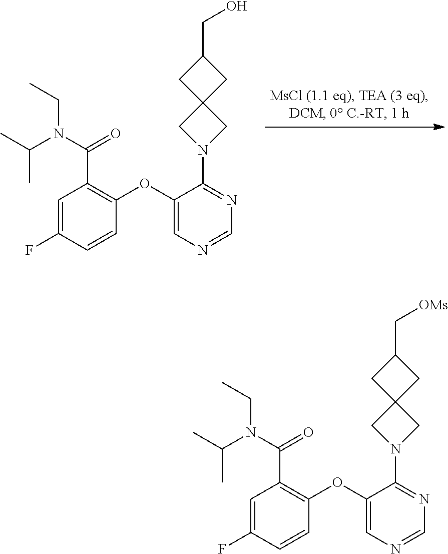 [SMILES: CCN(C(=O)c1cc(F)ccc1Oc1cncnc1N1CC2(CC(CO)C2)C1)C(C)C ; CCN(C(=O)c1cc(F)ccc1Oc1cncnc1N1CC2(CC(COS(C)(=O)=O)C2)C1)C(C)C]

To a stirred solution of N-ethyl-5-fluoro-2-((4-(6-(hydroxymethyl)-2-azaspiro[3.3]heptan-2-yl)pyrimidin-5-yl)oxy)-N-isopropylbenzamide (2 g, 4.67 mmol) in DCM (20 mL) under a nitrogen atmosphere, TEA (1.312 mL, 9.33 mmol) and MsCl (0.397 mL, 5.13 mmol) were added at 0° C. The reaction was stirred at RT for 1 h, and monitored by TLC (10% MeOH in DCM). After completion, the reaction was quenched with water (20 mL), and the mixture was extracted with DCM (2×50 mL). The organic layer was washed with brine solution (50 mL), dried over anhydrous sodium sulfate and filtered, and the filtrate was concentrated under reduced pressure to obtain crude (2-(5-(2-(ethyl(isopropyl)carbamoyl)-4-fluorophenoxy)pyrimidin-4-yl)-2-azaspiro[3.3]heptan-6-yl)methyl methanesulfonate (1.5 g, 62.4% yield) as a solid. This material was used without further purification. LCMS (Method F): Rt 1.49 min, m/z=507.2 [M+H]⁺, 98.40%.

### Intermediate 14. 5-Fluoro-2-((4-(6-formyl-2-azaspiro[3.3]heptan-2-yl)pyrimidin-5-yl)oxy)-N,N-diisopropylbenzamide

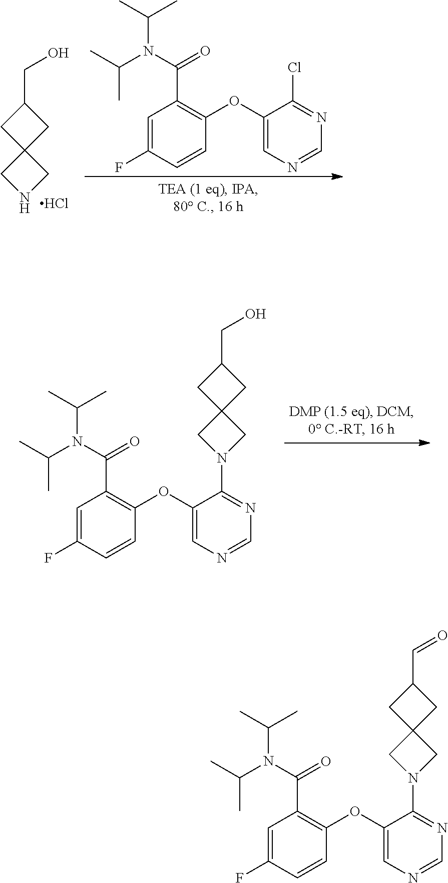

### Step 1. 5-Fluor-2-((4-(6-(hydroxymethyl)-2-azaspiro[3.3]heptan-2-yl)pyrimidin-5-yl)oxy)-N,N-diisopropylbenzamide

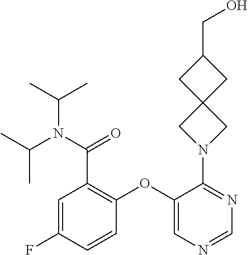 [SMILES: CC(C)N(C(=O)c1cc(F)ccc1Oc1cncnc1N1CC2(CC(CO)C2)C1)C(C)C]

This compound was synthesized following the procedure described for the synthesis of Intermediate 11, starting with 1 equivalent of 2-((4-chloropyrimidin-5-yl)oxy)-5-fluoro-N,N-diisopropylbenzamide and 1 equivalent of (2-azaspiro[3.3]heptan-6-yl)methanol hydrochloride. The reaction was stirred at 80° C. After completion, the reaction was concentrated under reduced pressure. The crude product was purified by silica gel flash column chromatography using MeOH in DCM (product eluted at 5% MeOH in DCM) to afford 5-fluoro-2-((4-(6-(hydroxymethyl)-2-azaspiro[3.3]heptan-2-yl)pyrimidin-5-yl)oxy)-N,N-diisopropylbenzamide as a gummy liquid.

Yield: 61.4%; ¹H NMR (400 MHz, DMSO-d₆): δ 8.26 (s, 1H), 7.72 (s, 1H), 7.26-7.19 (m, 2H), 7.04-6.98 (m, 1H), 4.49 (t, J=: 5.3 Hz, 1H), 4.23-4.15 (m, 1H), 4.13-3.97 (m, 3H), 3.68 (spt, J=6.5 Hz, 1H), 3.53 (spt, J=6.8 Hz, 1H), 3.31-3.28 (m, 2H), 2.24-2.10 (m, 3H), 1.94-1.86 (m, 2H), 1.45 (d, J=6.7 Hz, 3H), 1.35 (d, J=6.7 Hz, 3H), 1.09 (d, J=6.6 Hz, 3H), 1.00 (d, J=6.6 Hz, 3H); LCMS (Method B): Rt=1.80 min, m/z=443.2 [M+H]⁺, 96.93%.

### Step 2. 5-Fluoro-2-((4-(6-formyl-2-azaspiro[3.3]heptan-2-yl)pyrimidin-5-yl)oxy)-N,N-diisopropylbenzamide

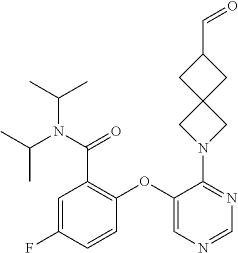 [SMILES: CC(C)N(C(=O)c1cc(F)ccc1Oc1cncnc1N1CC2(CC(C=O)C2)C1)C(C)C]

This compound was synthesized following the procedure described for the synthesis of Intermediate 12, starting with 1 equivalent of 5-fluoro-2-((4-(6-(hydroxymethyl)-2-azaspiro[3.3]heptan-2-yl)pyrimidin-5-yl)oxy)-N,N-diisopropylbenzamide. After work-up, the filtrate was concentrated under reduced pressure to afford crude 5-fluoro-2-((4-(6-formyl-2-azaspiro[3.3]heptan-2-yl)pyrimidin-5-yl)oxy)-N,N-diisopropylbenzamide as a liquid. This material was used without further purification.

Yield: 37%; LCMS (Method D): Rt=1.79 min, m/z=441.2 [M+H]⁺, 40.89%.

### Intermediate 15. Benzyl 6-oxo-2-azaspiro[3.3]heptane-2-carboxylate

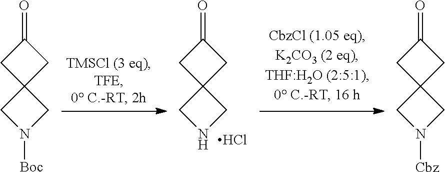 [SMILES: CC(C)(C)OC(=O)N1CC2(CC(=O)C2)C1 ; O=C1CC2(CNC2)C1 ; Cl ; O=C1CC2(C1)CN(C(=O)OCc1ccccc1)C2]

### Step 1. 2-Azaspiro[3.3]heptan-6-one hydrochloride

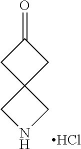

In a 50 mL round bottom flask under a nitrogen atmosphere, tert-butyl 6-oxo-2-azaspiro[3.3]heptane-2-carboxylate (6 g, 28.4 mmol) was dissolved in TFE (50 mL). TMSCl (10.89 mL, 85 mmol) was added at 0° C., and the reaction was stirred at RT for 2 h. The reaction was monitored by TLC (10% EtOAc in hexane). After completion, the reaction was concentrated under reduced pressure to obtain crude 2-azaspiro[3.3]heptan-6-one hydrochloride (4 g, 95% yield) as a solid. This material was used without further purification. ¹H NMR (400 MHz, DMSO-d₆): δ 9.06 (br s, 2H), 4.13 (s, 4H), 3.35 (s, 4H).

### Step 2. Benzyl 6-oxo-2-azaspiro[3.3]heptane-2-carboxylate

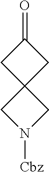 [SMILES: O=C1CC2(C1)CN(C(=O)OCc1ccccc1)C2]

In 250 mL round bottom flask under a nitrogen atmosphere, 2-azaspiro[3.3]heptan-6-one hydrochloride (4.74 g, 32.1 mmol) was dissolved in THF (27.5 mL), and the solution was cooled to 0° C. To this solution, K₂CO₃ (8.88 g, 64.2 mmol) in water (II mL) was added, then Cbz-Cl (4.79 mL, 33.7 mmol) was added dropwise. The reaction was stirred at RT for 16 h, and monitored by TLC (50% EtOAc in hexane). After completion, the reaction was diluted with water (60 mL) and the mixture was extracted EtOAc (200 mL). The combined organic layer was washed with water (2×100 mL), dried over anhydrous sodium sulfate and filtered, and the filtrate was concentrated under reduced pressure. The residue was purified by silica gel flash column chromatography using EtOAc in hexane (product eluted at 50% EtOAc in hexane) to afford benzyl 6-oxo-2-azaspiro[3.3]heptane-2-carboxylate (5.53 g, 65.2% yield) as a liquid. ¹H NMR (400 MHz, DMSO-d₆): δ 7.42-7.28 (m, 5H), 5.04 (s, 2H), 4.14 (br s, 4H), 3.31 (s, 4H); LCMS (Method D): Rt=1.60 min, m/z=244.1 [M−H]⁻, 92.92%.

### Intermediate. 16. N-Ethyl-5-fluoro-N-isopropyl-2-((4-(6-oxo-2-azaspiro[3.3]heptan-2-yl)pyrimidin-5-yl)oxy)benzamide

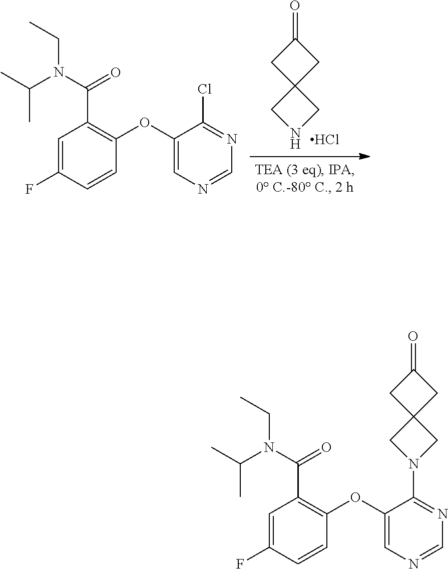 [SMILES: CCN(C(=O)c1cc(F)ccc1Oc1cncnc1Cl)C(C)C ; O=C1CC2(CNC2)C1 ; Cl ; CCN(C(=O)c1cc(F)ccc1Oc1cncnc1N1CC2(CC(=O)C2)C1)C(C)C]

To a stirred solution of 2-azaspiro[3.3]heptan-6-one hydrochloride (215 mg, 1.457 mmol) in IPA (10 mL), TEA (0.614 mL, 4.37 mmol) was added at 0° C. under a nitrogen atmosphere. The reaction was stirred at the same temperature for 5 min. To this reaction mixture, 2-((4-chloropyrimidin-5-yl)oxy)-N-ethyl-5-fluoro-N-isopropylbenzamide (492 mg, 1.457 mmol) was added at RT. The reaction was stirred at 80° C. for 2 h, and monitored by LCMS. After completion, the reaction was concentrated under reduced pressure. The crude compound was triturated with EtOAc (10 mL). The organic layer was decanted and the solid was dried under vacuum to obtain crude N-ethyl-5-fluoro-N-isopropyl-2-((4-(6-oxo-2-azaspiro[3.3]heptan-2-yl)pyrimidin-5-yl)oxy)benzamide (220 mg, 35.5% yield) as a solid. This material was used without further purification. LCMS (Method D): Rt=1.55 min, m/z=413.1 [M+H]⁺, 97.83%.

### Intermediate 17. (±)-(2-Azaspiro[3.4]octan-6-yl)methanol hydrochloride

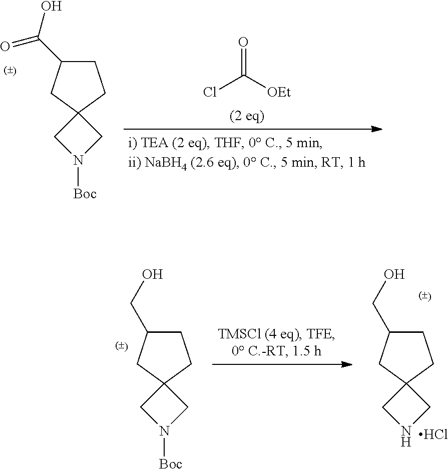 [SMILES: CC(C)(C)OC(=O)N1CC2(CCC(C(=O)O)C2)C1 ; CCOC(=O)Cl ; CC(C)(C)OC(=O)N1CC2(CCC(CO)C2)C1 ; OCC1CCC2(CNC2)C1 ; Cl]

### Step 1. (±)-tert-Butyl 6-(hydroxymethyl)-2-azaspiro[3.4]octane-2-carboxylate

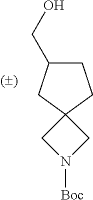 [SMILES: CC(C)(C)OC(=O)N1CC2(CCC(CO)C2)C1]

The reaction was performed following the same procedure in two batches (0.1 g×0.9 g).

To a stirred solution of (±)-2-(tert-butoxycarbonyl)-2-azaspiro[3.4]octane-6-carboxylic acid (0.9 g, 3.53 mmol) in dry THF (30 mL) under a nitrogen atmosphere, TEA (0.983 mL, 7.05 mmol) was added at 0° C., and the reaction was stirred for 5 min. To this reaction mixture, ethyl chloroformate (0.671 mL, 7.05 mmol) was added at 0° C., and the reaction was stirred at the same temperature for 30 min. The solid was filtered, and the filtrate was concentrated under reduced pressure to afford crude mixed anhydride (1.1 g). This material was dissolved in THF (30 mL), and a solution of NaBH₄ (0.318 g, 8.40 mmol) in water (10 mL) was added at 0° C. under a nitrogen atmosphere. The reaction was stirred at the same temperature for 5 min, and then at RT for 1 h. The reaction was monitored by TLC (50% EtOAc in hexane). After completion, the reaction was concentrated under reduced pressure and quenched with saturated sodium chloride (50 mL), and the mixture was extracted with EtOAc (2×50 mL). The combined organic layer was washed with brine (50 mL), dried over anhydrous sodium sulfate and filtered, and the filtrate was concentrated under reduced pressure. The crude materials from both batches (after work-up) were combined and purified by silica gel flash column chromatography using EtOAc in hexane (product eluted at 80% EtOAc in hexane) to afford (±)-tert-butyl 6-(hydroxymethyl)-2-azaspiro[3.4]octane-2-carboxylate (0.798 g, 98% combined yield) as a gum. ¹H NMR (400 MHz, DMSO-d₆): δ 4.51-4.44 (m, 1H), 3.73-3.58 (m, 4H), 3.27-3.14 (m, 2H), 2.10-1.97 (m, 1H), 1.88-1.79 (m, 1H), 1.76-1.69 (m, 2H), 1.69-1.59 (m, 1H), 1.49-1.43 (m, 1H), 1.37 (s, 9H), 1.32-1.23 (m, 11H); LCMS-ELSD (Method B): Rt=2.28 min, m/z=142.3 [M+H−100]⁺, 99.98%.

### Step 2. (±)-(2-Azaspiro[3.4]octan-6-yl)methanol hydrochloride

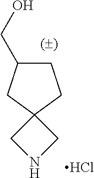

This compound was synthesized following the procedure described for the synthesis of Intermediate 15, Step 1, starting with 1 equivalent of (i)-tert-butyl 6-(hydroxymethyl)-2-azaspiro[3.4]octane-2-carboxylate, except that 4 equivalents of TMSCl was used. After completion, the reaction was concentrated under reduced pressure to afford crude (+)-(2-azaspiro[3.4]octan-6-yl)methanol hydrochloride as a gum. This material was used without further purification.

Yield: quantitative; ¹H NMR (400 MHz, DMSO-d₆): δ 8.90-8.62 (m, 2H), 4.53 (t, J=5.1 Hz, 1H), 3.83-3.70 (m, 4H), 3.30-3.18 (m, 2H), 2.08-1.91 (m, 2H), 1.89-1.73 (m, 2H), 1.70-1.58 (m, 1H), 1.56-1.47 (m, 1H), 1.34-1.24 (m, 1H).

### Intermediate 18. (±)-(2-(5-(2-(Ethyl(isopropyl)carbamoyl)-4-fluorophenoxy)pyrimidin-4-yl)-2-azaspiro[3.4]octan-6-yl)methyl 4-methylbenzenesulfonate

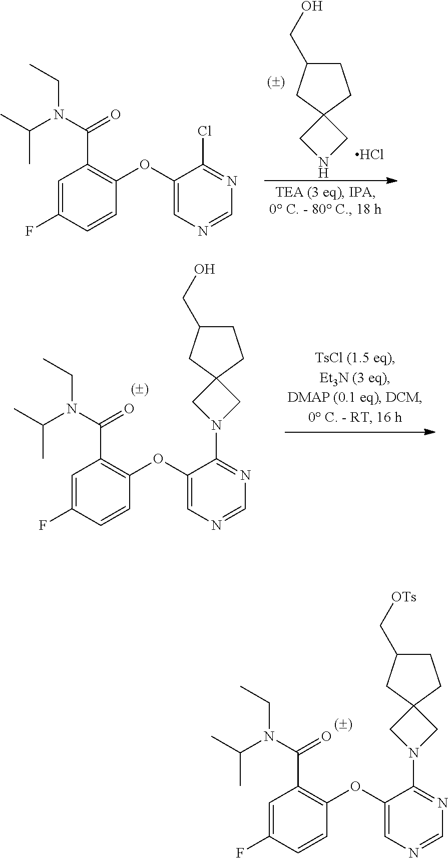

### Step 1. (±)-N-Ethyl-5-fluoro-2-((4-(6-(hydroxymethyl)-2-azaspiro[3.4]octan-2-yl)pyrimidin-5-yl)oxy)-N-isopropylbenzamide

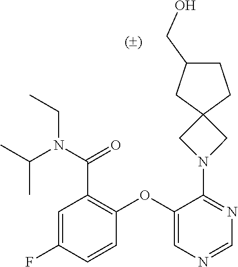 [SMILES: CCN(C(=O)c1cc(F)ccc1Oc1cncnc1N1CC2(CCC(CO)C2)C1)C(C)C]

This compound was synthesized following the procedure described for the synthesis of Intermediate 11, starting with 1 equivalent of 2-((4-chloropyrimidin-5-yl)oxy)-N-ethyl-5-fluoro-N-isopropylbenzamide and 1 equivalent of (±)-(2-azaspiro[3.4]octan-6-yl)methanol hydrochloride. The reaction was stirred at 80° C. After work-up, the crude compound was purified by silica gel flash column chromatography using MeOH in DCM (product eluted at 5-6% MeOH in DCM) to afford (±)-N-ethyl-5-fluoro-2-((4-(6-(hydroxymethyl)-2-azaspiro[3.4]octan-2-yl)pyrimidin-5-yl)oxy)-N-isopropylbenzamide as a gum.

Yield: 87%; ¹H NMR (400 MHz, DMSO-d₆): δ 8.33-8.20 (m, 1H), 7.77-7.63 (m, 1H), 7.33-7.19 (m, 2H), 7.10-6.99 (m, 1H), 4.51-4.43 (m, 1H), 4.08-3.88 (m, 4H), 3.81-3.68 (m, 11H), 3.46-3.37 (m, 1H), 3.30-3.10 (m, 3H), 2.11-1.98 (m, 1H), 1.94-1.84 (m, 1H), 1.80-1.59 (m, 3H), 1.54-1.43 (m, 1H), 1.36-1.24 (m, 1H), 1.23-0.96 (m, 9H); LCMS (Method D): Rt=1.71 min, m/z=443.4 [M+H]⁺, 99.59%.

### Step 2. (±)-(2-(S-(2-(Ethyl(isopropyl)carbamoyl)-4-fluorophenoxy)pyrimidin-4-yl)-2-azaspiro[3.4]octan-6-yl)methyl 4-methylbenzenesulfonate

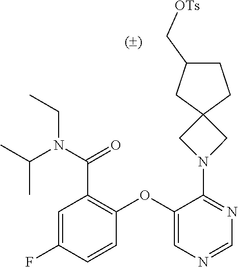 [SMILES: CCN(C(=O)c1cc(F)ccc1Oc1cncnc1N1CC2(CCC(COS(=O)(=O)c3ccc(C)cc3)C2)C1)C(C)C]

In a dried, 50 mL two neck round bottom flask under a nitrogen atmosphere, (±)-N-ethyl-5-fluoro-2-((4-(6-(hydroxymethyl)-2-azaspiro[3.4]octan-2-yl)pyrimidin-5-yl)oxy)-N-isopropylbenzamide (500 mg, 1.130 mmol) was dissolved in DCM (7 mL). To this solution, TEA (0.472 mL, 3.39 mmol) and DMAP (13.80 mg, 0.113 mmol) were added at 0° C. After 5 min, a solution of TsCl (323 mg, 1.695 mmol) in DCM (2 mL) was added dropwise at 0° C., and the reaction was stirred at RT for 16 h. The reaction progress was monitored by TLC (80% EtOAc in hexane). After completion, the reaction was quenched with water (60 mL) and the mixture was extracted with DCM (3×50 mL). The combined organic layer was washed with brine (60 mL), dried over anhydrous sodium sulfate and filtered, and the filtrate was concentrated under reduced pressure. The crude product was purified by silica gel flash column chromatography using EtOAc in hexane (product eluted at 76% EtOAc in hexane) to obtain (±)-(2-(5-(2-(ethyl(isopropyl)carbamoyl)-4-fluorophenoxy)pyrimidin-4-yl)-2-azaspiro[3.4]octan-6-yl)methyl 4-methylbenzenesulfonate (400 mg, 59.1% yield) as a sticky solid. LCMS (Method D): Rt=2.20 min, m/z=597.4 [M+H]⁺, 99.55%.

### Intermediate 19. tert-Butyl (4aS,8aS)-4-((2-(5-(4-fluoro-2-(methoxycarbonyl)phenoxy)pyrimidin-4-yl)-2-azaspiro[3.3]heptan-6-yl)methyl)hexahydro-2H-pyrido[4,3-b][1,4]oxazine-6(5H)-carboxylate

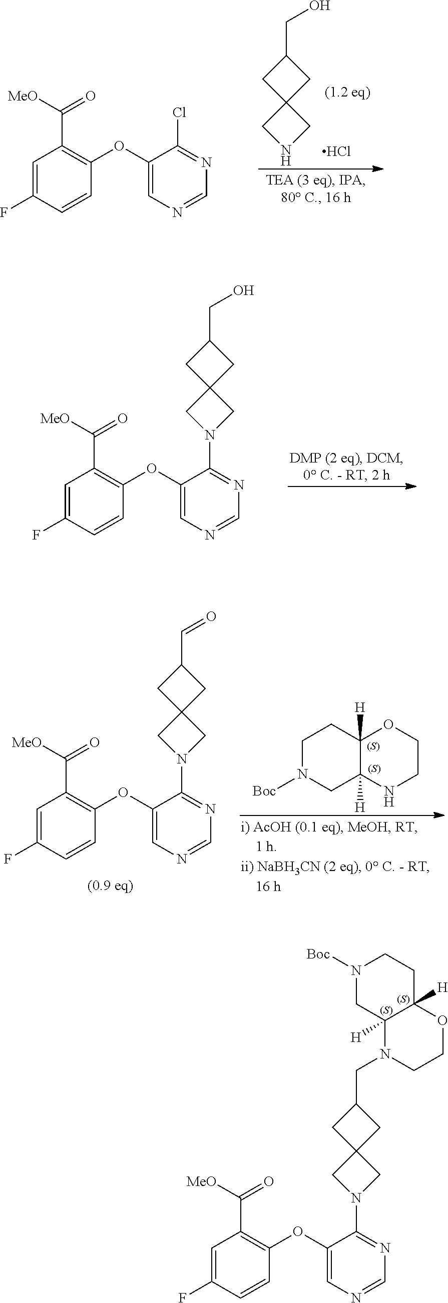

### Step 1. Methyl 5-fluoro-2-((4-(6-(hydroxymethyl)-2-azaspiro[3.3]heptan-2-yl)pyrimidin-5-yl)oxy)benzoate

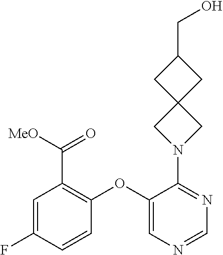 [SMILES: COC(=O)c1cc(F)ccc1Oc1cncnc1N1CC2(CC(CO)C2)C1]

This compound was synthesized following the procedure described for the synthesis of Intermediate 11, starting with 1 equivalent of methyl 2-((4-chloropyrimidin-5-yl)oxy)-5-fluorobenzoate and 1.2 equivalents of (2-azaspiro[3.3]heptan-6-yl)methanol hydrochloride. The reaction was stirred at 80° C. After completion, the reaction was concentrated under reduced pressure. The residue was dissolved in DCM, and the mixture was washed with water. The combined organic layer was washed with brine, dried over anhydrous sodium sulfate and filtered, and the filtrate was concentrated under reduced pressure. The crude product was purified by silica gel flash column chromatography using MeOH in DCM (product eluted at 5% MeOH in DCM) to afford methyl 5-fluoro-2-((4-(6-(hydroxymethyl)-2-azaspiro[3.3]heptan-2-yl)pyrimidin-5-yl)oxy)benzoate as a gum.

Yield: 76%; ¹H NMR (400 MHz, DMSO-d₆): δ 8.28 (s, 111), 7.69-7.63 (m, 2H), 7.51-7.43 (m, 1H), 7.07 (dd, J=4.5, 9.1 Hz, 1H), 4.49 (t, J=5.3 Hz, 1H), 4.16 (s, 2H), 4.07 (s, 2H), 3.81 (s, 3H), 2.24-2.13 (m, 4H), 1.96-1.86 (m, 2H), one proton merged with solvent peaks; LCMS (Method E): Rt=1.59 min, m/z=374.2 [M+H]⁺, 99.77%.

### Step 2. Methyl 5-fluoro-2-((4-(6-formyl-2-azaspiro[3.3]heptan-2-yl)pyrimidin-5-yl)oxy)benzoate

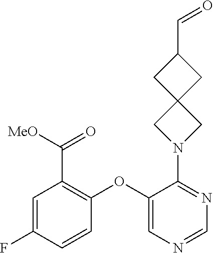 [SMILES: COC(=O)c1cc(F)ccc1Oc1cncnc1N1CC2(CC(C=O)C2)C1]

This compound was synthesized following the procedure described for the synthesis of Intermediate 12, starting with 1 equivalent of methyl 5-fluoro-2-((4-(6-(hydroxymethyl)-2-azaspiro[3.3]heptan-2-yl)pyrimidin-5-yl)oxy)benzoate, except that 2 equivalents of DMP was used. After completion, the reaction was filtered through a Celite® pad, the pad was washed with DCM, and the filtrate was washed with saturated sodium bicarbonate solution. The organic layer was dried over anhydrous sodium sulfate and filtered, and the filtrate was concentrated under reduced pressure to afford crude methyl 5-fluoro-2-((4-(6-formyl-2-azaspiro[3.3]heptan-2-yl)pyrimidin-5-yl)oxy)benzoate as a gum. This material was used without further purification.

Yield: 35.3%; LCMS (Method B): Rt=1.28-1.34 min, m/z=372.2 [M+H]⁺, 48.96%.

### Step 3. tert-Butyl (4aS,8aS)-4-((2-(5-(4-fluoro-2-(methoxycarbonyl)phenoxy)pyrimidin-4-yl)-2-azaspiro[3.3]heptan-6-yl)methyl)hexahydro-2H-pyrido[4,3-b][1,4]oxazine-6(5H)-carboxylate

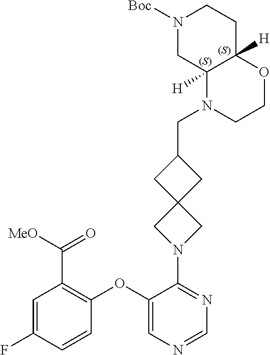

To a stirred solution of methyl 5-fluoro-2-((4-(6-formyl-2-azaspiro[3.3]heptan-2-yl)pyrimidin-5-yl)oxy)benzoate (2.62 g, 7.06 mmol) and tert-butyl (4aS,8aS)-hexahydro-2H-pyrido[4,3-b][1,4]oxazine-6(5H)-carboxylate (1.9 g, 7.84 mmol) in MeOH (40 mL), AcOH (0.045 mL, 0.784 mmol) was added at 0° C. The reaction was stirred at RT for 1 h. To this reaction mixture, sodium cyanoborohydride (0.985 g, 15.68 mmol) was added at 0° C., and the reaction was stirred at RT for 16 h. The reaction progress was monitored by TLC (10% MeOH in DCM). After completion, the reaction was concentrated under reduced pressure. The residue was diluted with 10% MeOH in DCM (20 mL), and the mixture was washed with water (20 mL). The organic layer was dried over anhydrous sodium sulfate and filtered, and the filtrate was concentrated under reduced pressure. The crude was purified by silica gel flash column chromatography using MeOH in DCM (product eluted at 5% MeOH in DCM) to afford tert-butyl (4aS,8aS)-4-((2-(5-(4-fluoro-2-(methoxycarbonyl)phenoxy)pyrimidin-4-yl)-2-azaspiro[3.3]heptan-6-yl)methyl)hexahydro-2H-pyrido[4,3-b][1,4]oxazine-6(5H)-carboxylate (1.7 g, 33.8% yield) as a sticky solid.

¹H NMR (400 MHz, DMSO-d₆): δ 8.28 (s, 1H), 7.70-7.64 (m, 2H), 7.47 (ddd, J=3.2, 7.8, 9.1 Hz, 1H), 7.07 (dd, J=4.4, 9.1 Hz, 1H), 423-4.15 (m, 3H), 4.11-4.04 (m, 2H), 4.01-3.88 (m, 1H), 3.81 (s, 3H), 3.75-3.70 (m, 1H), 3.54-3.47 (m, 2H), 3.17-3.10 (m, 1H), 2.31-2.22 (m, 4H), 1.90-1.81 (m, 2H), 1.79-1.69 (m, 2H), 1.39 (m, 13H), one proton merged with solvent peaks; LCMS (Method E): Rt=2.15 min, m/z=598.2 [M+H]⁺, 93.13%.

### Intermediate 20. Benzyl ((1r,3r)-3-amino-3-methylcyclobutyl)(isopropyl)carbamate

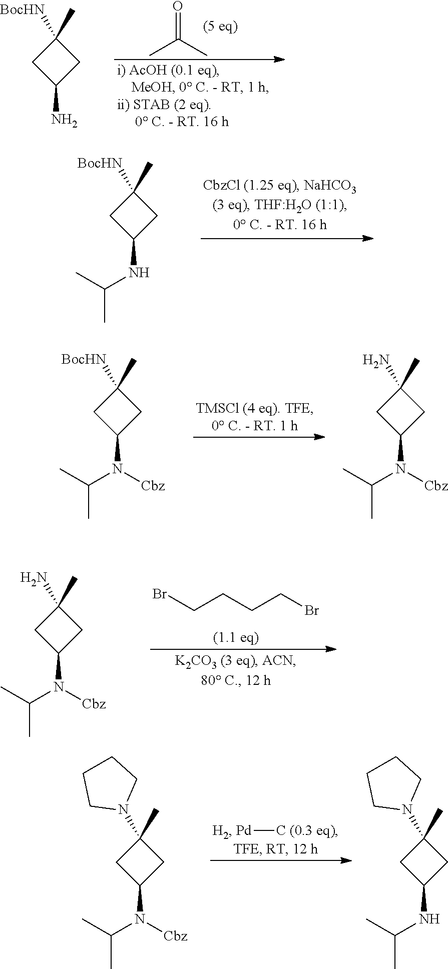

### Step 1. tert-Butyl ((1r, 3r)-3-(isopropylamino)-1-methylcyclobutyl)carbamate

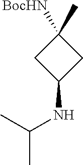 [SMILES: CC(C)N[C@H]1C[C@](C)(NC(=O)OC(C)(C)C)C1]

In a 10 L two neck dried round bottom flask under a nitrogen atmosphere, tert-butyl ((1r,3r)-3-amino-1-methylcyclobutyl)carbamate (25 g, 125 mmol) was dissolved in MeOH (400 mL). To this solution, acetone (45.8 mL, 624 mmol) and AcOH (0.715 mL, 12.48 mmol) were added at 0° C., and the reaction was stirred at RT for 1 h. Sodium triacetoxyborohydride (52.9 g, 250 mmol) was then added at 0° C., and the reaction was stirred at RT for 16 h. The reaction was monitored by TLC (10% MeOH in DCM). After completion, the reaction was concentrated under reduced pressure. The crude was dissolved in saturated sodium bicarbonate solution (500 mL) and the mixture was extracted with DCM (3×500 mL). The combined organic layer was dried over anhydrous sodium sulfate and filtered, and the filtrate was concentrated under reduced pressure to afford crude tert-butyl (3-(isopropylamino)-1-methylcyclobutyl)carbamate (30 g, 99% yield) as a semi-solid. This material was used without further purification. ¹H NMR (400 MHz, DMSO-d₆): δ 6.87 (br s, 1H), 3.28 (quin, J=7.7 Hz, 11H), 2.76 (spt, J=6.2 Hz, 1H), 2.48-2.35 (m, 2H), 1.69-1.60 (m, 2H), 1.38 (s, 9H), 1.29 (s, 3H), 0.96 (d, J=6.3 Hz, 6H), one proton merged with solvent peaks.

### Step 2. Benzyl ((1r,3r)-3-((tert-butoxycarbonyl)amino)-3-methylcyclobutyl)(isopropyl)carbamate

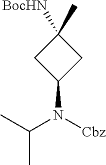 [SMILES: CC(C)N(C(=O)OCc1ccccc1)[C@H]1C[C@](C)(NC(=O)OC(C)(C)C)C1]

The reaction was performed following the same procedure in four individual batches (20 g, 20 g, 25 g, and 27 g).

To a stirred solution of tert-butyl ((1r,3r)-3-isopropylamino)-1-methylcyclobutyl)carbamate (25 g, 103 mmol) in THF (250 mL) and water (250 mL), solid sodium bicarbonate (26.0 g, 309 mmol) and Cbz-Cl (22.00 g, 129 mmol) were added at 0° C. The reaction was stirred at RT for 16 h. The reaction progress was monitored by TLC (30% EtOAc in hexane). After completion, the reaction was cooled to 0° C. and quenched with saturated sodium bicarbonate solution (100 mL), and the mixture was extracted with EtOAc (3×60 mL). The combined organic layer was dried over anhydrous sodium sulfate and filtered, and the filtrate was concentrated under reduced pressure. The crude was purified by silica gel flash column chromatography using EtOAc in hexane (product was eluted at 12% EtOAc in hexane) to obtain benzyl ((1r,3r)-3-((tert-butoxycarbonyl)amino)-3-methylcyclobutyl)(isopropyl)carbamate (28 g, 72% yield) as a solid. LCMS (Method E): Rt=2.34 min, m/z=377.2 [M+H]⁺, 90.38%.

### Step 3. Benzyl ((1r,3r)-3-amino-3-methylcyclobutyl)(isopropyl)carbamate

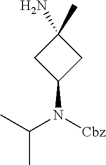 [SMILES: CC(C)N(C(=O)OCc1ccccc1)[C@H]1C[C@](C)(N)C1]

In a dried, 2 L two neck round bottom flask under a nitrogen atmosphere, benzyl ((1r,3r)-3-((tert-butoxycarbonyl)amino)-3-methylcyclobutyl)(isopropyl)carbamate (100 g, 266 mmol) was dissolved in TFE (1000 mL). To this solution, TMSCl (135 mL, 1062 mmol) was added at 0° C. and the reaction was stirred at RT for 1 h. The reaction progress was monitored by TLC (10% MeOH in DCM). After completion, the reaction was concentrated under reduced pressure to obtain benzyl ((1r,3r)-3-amino-3-methylcyclobutyl)(isopropyl)carbamate hydrochloride. The residue was basified using 10% NaHCO₃ solution (600 mL) and the mixture was extracted with 10% MeOH in DCM (3×700 mL). The combined organic layer was washed with brine solution, dried over anhydrous sodium sulfate, and filtered. The filtrate was concentrated under reduced pressure to obtain crude benzyl ((1r,3r)-3-amino-3-methylcyclobutyl)(isopropyl)carbamate (73.2 g, 99% yield) as a liquid. This material was used without further purification. LCMS (Method G): Rt=1.44 min, m/z=277.1 [M+H]⁺, 99.75%.

### Step 4. Benzyl isopropyl((1r,3r)-3-methyl-3-(pyrrolidin-1-yl)cyclobutyl)carbamate

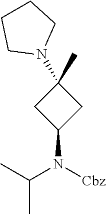 [SMILES: CC(C)N(C(=O)OCc1ccccc1)[C@H]1C[C@](C)(N2CCCC2)C1]

To a stirred solution of benzyl ((1r,3r)-3-amino-3-methylcyclobutyl)(isopropyl)carbamate (30 g, 109 mmol) in ACN (150 mL), K₂CO₃ (45.0 g, 326 mmol) and 1,4-dibromobutane (14.26 mL, 119 mmol) were added at RT. The reaction was stirred at 80° C. for 12 h. The reaction progress was monitored by TLC (10% MeOH in DCM). After completion, the reaction was quenched with 10% NaHCO₃ solution (500 mL) and the mixture was extracted with EtOAc (2×500 mL). The combined organic layer was washed with brine solution (200 mL), dried over anhydrous sodium sulfate, and filtered. The filtrate was concentrated under reduced pressure to obtain crude benzyl isopropyl((1r,3r)-3-methyl-3-(pyrrolidin-1-yl)cyclobutyl)carbamate (31 g, 86% yield) as an oily liquid. This material was used without further purification. LCMS (Method B): Rt=1.40 min, m/z=331.2 [M+H]⁺, 99.76%.

### Step 5. (1r,3r)-N-Isopropyl-3-methyl-3-pyrrolidin-1-yl)cyclobutan-1-amine

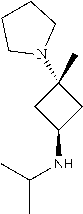

In a 500 mL round bottom flask under a nitrogen atmosphere, benzyl isopropyl((1r,3r)-3-methyl-3-(pyrrolidin-1-yl)cyclobutyl)carbamate (25 g, 76 mmol) was dissolved in TFE (300 mL). To this solution, 10% Pd—C (24.15 g, 22.69 mmol) was added at RT, and the reaction was stirred for 12 h at RT under a hydrogen atmosphere (balloon pressure). The reaction progress was monitored by TLC (10% MeOH in DCM). After completion, the reaction was filtered through a Celite® pad, and the pad was washed with THF (600 mL), then with EtOAc (600 mL). The filtrate was concentrated under reduced pressure to obtain crude (1r,3r)-N-isopropyl-3-methyl-3-(pyrrolidin-1-yl)cyclobutan-1-amine (15 g, 86% yield) as a semi-solid. This material was used without further purification. This material was used without further purification. LCMS (Method B): Rt=0.18 min, m/z=197.3 [M+H]⁺, 85.46%.

### Intermediate 21. (±)-N-Ethyl-5-fluoro-2-((4-(6-formyl-2-azaspiro[3.4]octan-2-yl)pyrimidin-5-yl)oxy)-N-isopropylbenzamide

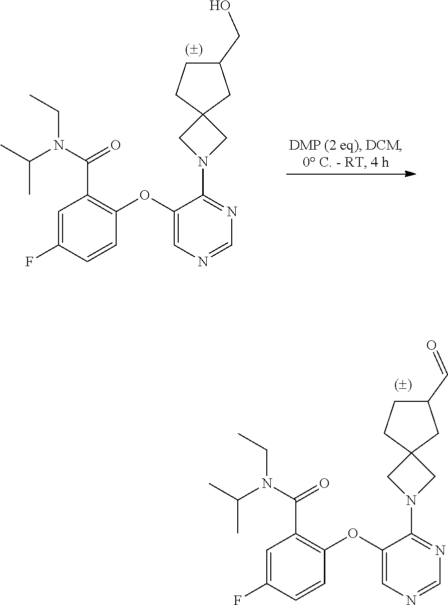 [SMILES: CCN(C(=O)c1cc(F)ccc1Oc1cncnc1N1CC2(CCC(CO)C2)C1)C(C)C ; CCN(C(=O)c1cc(F)ccc1Oc1cncnc1N1CC2(CCC(C=O)C2)C1)C(C)C]

In a dried, 25 mL round bottom flask under a nitrogen atmosphere, (±)-N-ethyl-5-fluoro-2-((4-(6-(hydroxymethyl)-2-azaspiro[3.4]octan-2-yl)pyrimidin-5-yl)oxy)-N-isopropylbenzamide (150 mg, 0.339 mmol) was dissolved in DCM (3 mL). To this solution, DMP (288 mg, 0.678 mmol) was added at 0° C. and the reaction was stirred at RT for 4 h. The reaction progress was monitored by TLC (110% EtOAc). After completion, the reaction was filtered through a Celite® pad, and the pad was washed with DCM (50 mL). The filtrate was washed with NaHCO₃ solution (2×50 mL) and brine solution (50 mL). The organic layer was dried over anhydrous sodium sulfate and filtered, and the filtrate was concentrated under reduced pressure to afford crude (i-N-ethyl-5-fluoro-2-((4-(6-formyl-2-azaspiro[3.4]octan-2-yl)pyrimidin-5-yl)oxy)-N-isopropylbenzamide (180 mg, 48.3% yield). This material was used without further purification.

LCMS (Method F): Rt=1.44 min, m/z=441.2 [M+H]⁺, 40.06%.

### Intermediate 22. tert-Butyl (2s,4r)-2-(isopropylamino)-5-azaspiro[3.4]octane-5-carboxylate

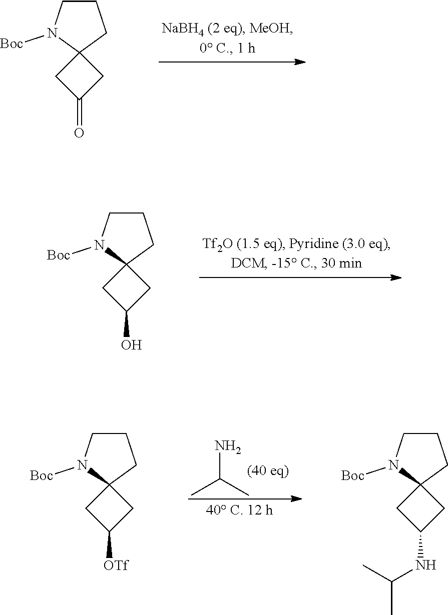

### Step 1. tert-Butyl (2r,4s)-2-hydroxy-5-azaspiro[3.4]octane-5-carboxylate

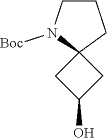 [SMILES: CC(C)(C)OC(=O)N1CCC[C@]12C[C@H](O)C2]

To a stirred solution of tert-butyl 2-oxo-5-azaspiro[3.4]octane-5-carboxylate (10 g, 44.4 mmol) in MeOH (100 mL) 0° C., NaBH₄ (3.36 g, 89 mmol) was added over 20 min. The reaction was stirred at the same temperature for 1 h, and monitored by TLC (30% EtOAc in hexane). After completion, the reaction was quenched with ice-cold water (150 mL) and the mixture was extracted with EtOAc (2×200 mL). The combined organic layer was washed with brine solution (100 mL), dried over anhydrous sodium sulfate and filtered, and the filtrate was concentrated under reduced pressure to obtain crude tert-butyl (2r,4s)-2-hydroxy-5-azaspiro[3.4]octane-5-carboxylate (14.2 g, quantitative yield) as a solid. This material was used without further purification. LCMS (Method E): Rt=1.68 min, m/z=172.1 [M+H−56]⁺, 98.69%.

### Step 2. tert-Butyl (2r,4s)-2-(((trifluoromethyl)sulfonyl)oxy)-5-azaspiro[3.4]octane-5-carboxylate

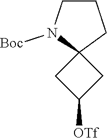 [SMILES: CC(C)(C)OC(=O)N1CCC[C@]12C[C@H](OS(=O)(=O)C(F)(F)F)C2]

To a stirred solution of tert-butyl (2r,4s)-2-hydroxy-5-azaspiro[3.4]octane-5-carboxylate (7 g, 30.8 mmol) in DCM (70 mL) and pyridine (7.46 mL, 92 mmol) at 0° C., Tf₂O (7.76 mL, 46.2 mmol) was added over 15 min. The reaction was stirred at 0° C. for 30 min, and monitored by TLC (30% EtOAc in hexane). After completion, the reaction was quenched with ice-cold water (70 mL) and the mixture was extracted with DCM (2×100 mL). The combined organic layer was washed with aqueous NaHCO₃ solution (70 mL), dried over anhydrous sodium sulfate and filtered, and the filtrate was concentrated under reduced pressure to afford crude tert-butyl (2r,4s)-2-(((trifluoromethyl)sulfonyl)oxy)-5-azaspiro[3.4]octane-5-carboxylate (10 g, 14.39 mmol, 46.7% yield) as a solid. This compound was used without further purification. LCMS (Method E): Rt=2.36 min, m/z=303.9 [M+H−56]⁺, 51.70%.

### Step 3. tert-Butyl (2s,4r)-2-(isopropylamino)-5-azaspiro[3.4]octane-5-carboxylate

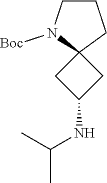 [SMILES: CC(C)N[C@H]1C[C@@]2(CCCN2C(=O)OC(C)(C)C)C1]

In 250 mL sealed tube, tert-butyl (2r,4s)-2-(((trifluoromethyl)sulfonyl)oxy)-5-azaspiro[3.4]octane-5-carboxylate (10 g, 27.8 mmol) was dissolved in isopropylamine (65.8 g, 1113 mmol), and the reaction was stirred at 40° C. for 12 h. The reaction progress was monitored by TLC (10% MeOH in DCM). After completion, the reaction was quenched with 10% NaHCO₃ solution (100 mL) and the mixture was extracted with 10%/6 MeOH in DCM (3×100 mL). The combined organic layer was washed with brine solution (100 mL), dried over anhydrous sodium sulfate and filtered, and the filtrate was concentrated under reduced pressure to afford crude (tert-butyl (2s,4r)-2-(isopropylamino)-5-azaspiro[3.4]octane-5-carboxylate (10.5 g, quantitative yield) as a gummy liquid. This material was used without further purification. ¹H NMR (400 MHz, DMSO-d₆): δ 3.59-3.53 (m, 1H), 3.45-3.35 (m, 1H), 3.23 (t, J=6.6 Hz, 2H), 2.95-2.66 (m, 3H), 2.09-1.98 (m, 2H), 1.84-1.73 (m, 2H), 1.60 (quin, J=6.6 Hz, 2H), 1.53-1.34 (m, 9H), 1.05-0.86 (m, 6H); LCMS (Method B): Rt=1.25 min, m/z=269.6 [M+H]⁺, 83.12%.

### Intermediate 23. tert-Butyl (2s,4r)-2-(isopropylamino)-5-azaspiro[3.5]nonane-5-carboxylate

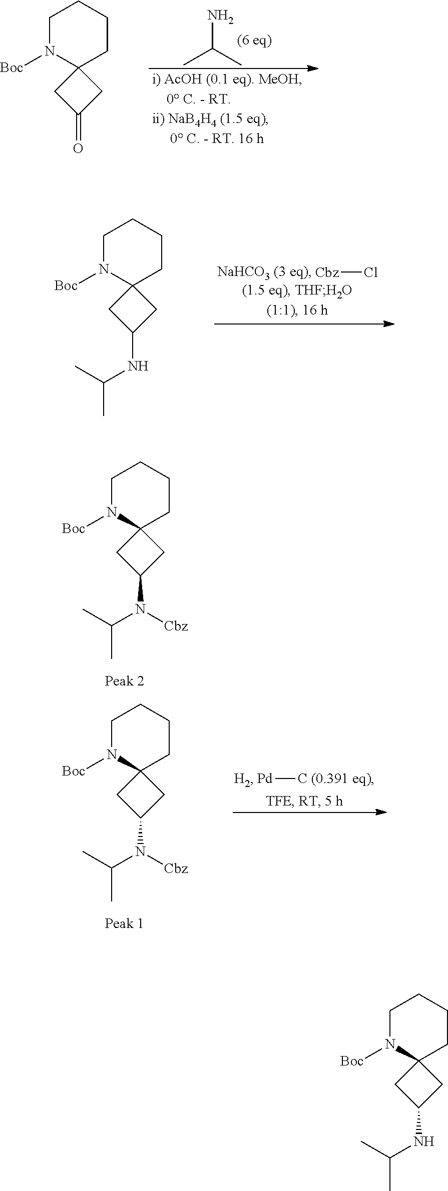

### Step 1. tert-Butyl 2-(isopropylamino)-5-azaspiro[3.5]nonane-5-carboxylate

In a 40 mL pressure vial, tert-butyl 2-oxo-5-azaspiro[3.5]nonane-5-carboxylate (1.0 g, 4.18 mmol) was dissolved in MeOH (3 mL) and the solution was cooled to 0° C. To this stirred solution, isopropylamine (2.148 mL, 25.07 mmol) and AcOH (0.024 mL, 0.418 mmol) were added. The reaction was stirred at RT for 16 h, then was re-cooled to 0° C., and NaBH₄ (0.237 g, 6.27 mmol) was added. The reaction was continued at 0° C. for 1 h, and progress was monitored by TLC (30% EtOAc in hexane). After completion, the reaction was concentrated under reduced pressure. The residue was diluted with water (30 mL) and the mixture was extracted with EtOAc (3×45 mL).

The combined organic layer was dried over sodium sulfate and filtered, and the filtrate was concentrated under reduced pressure to obtain crude tert-butyl 2-(isopropylamino)-5-azaspiro[3.5]nonane-5-carboxylate (1.1 g, 93% yield). This material was used without further purification. ¹H NMR (400 MHz, DMSO-d₆): δ 3.21-3.07 (m, 3H), 2.92-2.60 (m, 2H), 2.48-2.32 (m, 2H), 1.83-1.71 (m, 3H), 1.65-1.49 (m, 3H), 1.39 (s, 9H), 1.33-1.21 (m, 2H), 0.93 (dd, J=2.1, 6.2 Hz, 6H); LCMS (Method B): Rt=1.40 min, m/z=283.2 [M+H]⁺, 99.90%.

### Step 2. tert-Butyl (2s,4r)-2-(((benzyloxy)carbonyl)(isopropyl)amino)-5-azaspiro[3.5]nonane-5-carboxylate and tert-butyl (2r,4s)-2-(((benzyloxy)carbonyl)(isopopyl)amino)-5-azaspiro[3.5]nonane-5-carboxylate

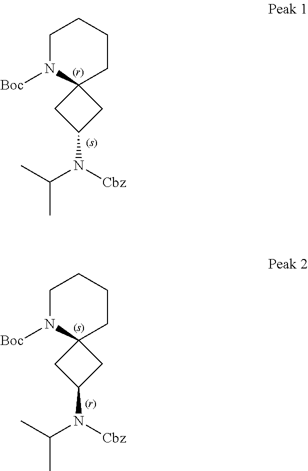 [SMILES: CC(C)N(C(=O)OCc1ccccc1)[C@H]1C[C@@]2(CCCCN2C(=O)OC(C)(C)C)C1 ; CC(C)N(C(=O)OCc1ccccc1)[C@H]1C[C@]2(CCCCN2C(=O)OC(C)(C)C)C1]

To a stirred solution of tert-butyl 2-(isopropylamino)-5-azaspiro[3.5]nonane-5-carboxylate (200 mg, 0.708 mmol) in 1:1 THF:H₂O (2 mL: 2 mL) at 0° C., sodium bicarbonate (178 mg, 2.124 mmol) and CbzCl (0.152 mL, 1.062 mmol) were added. The reaction was stirred at RT for 16 h, and monitored by TLC (30% EtOAc in hexane). After completion, the reaction was cooled to 0-5° C. and quenched with sodium bicarbonate solution (10 mL). The mixture was extracted with EtOAc (3×10 mL). The combined organic layer was washed with brine solution (30 mL), dried over sodium sulfate and filtered, and the filtrate was concentrated under reduced pressure. The residue was purified by silica gel flash column chromatography using EtOAc in hexane (product eluted at 12% EtOAc in hexane) to obtain both isomers.

Peak 1. tert-Butyl (2s,4r)-2-(((benzyloxy)carbonyl)(isopropyl)amino)-5-azaspiro[3.5]nonane-5-carboxylate (102 mg, 34.5% yield) as a gummy liquid. ¹H NMR (400 MHz, CDCl₃): δ 7.40-7.30 (m, 5H), 5.15 (s, 2H), 4.18 (Sep, J=6.8 Hz, 1H). 4.00 (quin, J=9.1 Hz, 1H), 3.42-3.37 (m, 2H), 2.85-2.73 (m, 2H), 2.48 (br t, J=10.6 Hz, 2H), 1.66-1.58 (m, 2H), 1.53-1.50 (m, 2H), 1.48 (s, 9H), 1.40-1.32 (m, 2H), 1.17 (d, J=6.8 Hz, 6H); LCMS (Method B): Rt=2.86 min, m/z=317.2 [M+H-Boc]⁺, 99.90%.

Peak 2. tert-Butyl (2r,4s)-2-(((benzyloxy)carbonyl)(isopropyl)amino)-5-azaspiro[3.5]nonane-5-carboxylate (35 mg, 11.85% yield) as a gummy liquid. ¹H NMR (400 MHz, DMSO-d₆): δ 7.39-7.29 (m, 5H), 5.07 (s, 2H), 3.97-3.86 (m, 1H), 3.83-3.72 (m, 1H), 3.15-3.08 (m. 2H), 2.32-2.23 (m, 2H), 1.67-1.56 (m, 4H), 1.37 (s, 9H), 1.33-1.26 (m, 2H), 1.15 (d, J=6.9 Hz, 6H), two protons merged with solvent peaks; LCMS (Method B): Rt=2.79 min, m/z=417.3 [M+H]⁺, 99.89%.

### Step 3. tert-Butyl (2s,4r)-2-(isopropylamino)-5-azaspiro[3.5]nonane-5-carboxylate

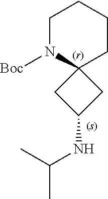 [SMILES: CC(C)N[C@H]1C[C@@]2(CCCCN2C(=O)OC(C)(C)C)C1]

In a 10 mL round bottom flask, tert-butyl (2s,4r)-2-(((benzyloxy)cabonyl)(isopropyl)amino)-5-azaspiro[3.5]nonane-5-carboxylate (90 mg, 0.216 mmol) was dissolved in TFE (4 mL), and Pd—C (90 mg, 0.085 mmol) was added at RT. The reaction was stirred at RT for 5 h under a hydrogen atmosphere (balloon pressure). The reaction progress was monitored by TLC (10% MeOH in DCM). After completion, the reaction was filtered through a Celite® pad, and the pad was washed with MeOH (2×20 mL). The filtrate was concentrated under reduced pressure, and the residue was reconcentrated from EtOAc (2×10 mL) then from toluene (2×10 mL) to obtain crude tert-butyl (2s,4r)-2-(isopropylamino)-5-azaspiro[3.5]nonane-5-carboxylate (46 mg, 74.8% yield) as a gummy liquid. This material was used without further purification. LCMS (Method B): Rt=1.36 min, m/z=283.2 [M+H]⁺, 99.23%.

### Example 1. N-Ethyl-5-fluoro-N-isopropyl-2-((4-(6-(((4S,8aS)-octahydro-4H-pyrido[4,3-b][1,4]oxazin-4-yl)methy)-2-azaspiro[3.3]heptan-2-yl)pyrimidin-5-yl)oxy)benzamide (Compound No. 1)

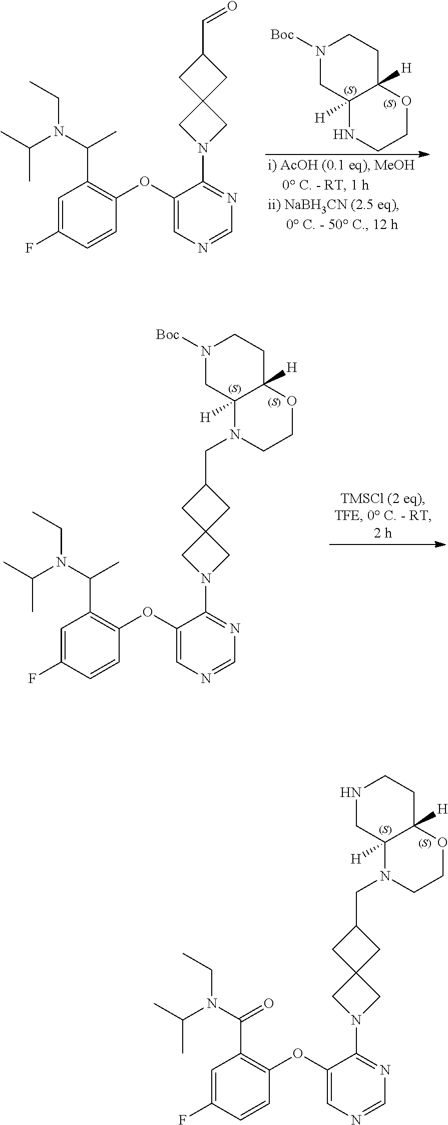

### Step 1. tert-Butyl (4aS,8aS)-4-((2-(S-(2-(ethyl(isopropyl)carbamoyl)-4-fluorophenoxy,)pyrimidin-4-yl)-2-azaspiro[3.3]heptan-6-yl)methyl)hexahydro-2H-pyrido[4,3-b][1,4]oxazine-6(5H)-carboxylate

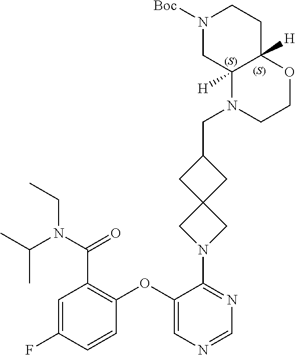

To a stirred solution of N-ethyl-5-fluoro-2-((4-(6-formyl-2-azaspiro[3.3]heptan-2-yl)pyrimidin-5-yl)oxy)-N-isopropylbenzamide (0.050 g, 0.117 mmol) and tert-butyl (4aR,8aS)-hexahydro-2H-pyrido[4,3-b][1,4]oxazine-6(5H)-carboxylate (0.028 g, 0.117 mmol) in MeOH (5 mL), acetic acid (0.677 μL, 0.012 mmol) was added, and the reaction was stirred at RT for 1 h. To this reaction mixture, NaBH₃CN (0.018 g, 0.293 mmol) was added at 0° C., and the reaction was stirred at 50° C. for 12 h. The reaction progress was monitored by TLC (10% MeOH in DCM). After completion, the reaction was concentrated under reduced pressure. The residue was diluted with water (50 mL) and the mixture was extracted with EtOAc (2×50 mL). The combined organic layer was washed with brine (50 mL), dried over anhydrous sodium sulfate and filtered, and the filtrate was concentrated under reduced pressure. The crude product (100 mg) was purified by silica gel flash column chromatography using MeOH in DCM (20% MeOH in DCM) to afford tert-butyl (4aS,8aS)-4-((2-(5-(2-(ethyl(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H₁)-carboxylate (70 mg, 58.5% yield) as a solid. LCMS (Method D): Rt=2.04 min, m/z=653.2 [M+H]⁺, 64.35%.

### Step 2. N-Ethyl-5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide

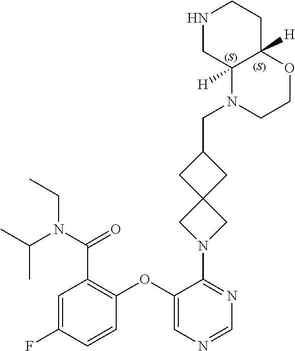

To a stirred solution of tert-butyl (4aS,8aS)-4-((2-(5-(2-(ethyl(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (115 mg, 0.176 mmol) in TFE (5 mL), TMSCl (0.045 mL, 0.352 mmol) was added at 0° C. The reaction was stirred at RT for 2 h, and monitored by TLC (5% MeOH in DCM). After completion, the reaction was concentrated under reduced pressure. The residue was dissolved in saturated sodium bicarbonate solution (30 mL), and the mixture was extracted with 10% MeOH in DCM (2×50 mL). The combined organic layer was washed with brine (50 mL), dried over anhydrous sodium sulfate and filtered, and the filtrate was concentrated under reduced pressure. The crude product (110 mg) was purified by reverse phase chromatography using a SiliCycle SiliaSep C18 column (40 g) on Biotage isolera one (Mobile phase A: 10 mM NH₄HCO₃ in water, B: ACN). The pure fractions were concentrated under reduced pressure and lyophilized to obtain N-ethyl-5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide (9.11 mg, 9.90%) as a solid. ¹H NMR (400 MHz, DMSO-d₄): δ 8.28-8.24 (m, 1H), 7.77-7.65 (m, 1H), 7.32-7.20 (m, 2H), 7.05-6.94 (m, 1H), 4.45-3.91 (m, 4H), 3.79-3.65 (m, 2H), 3.56-3.37 (m, 3H), 3.27-3.09 (m, 2H), 3.05-2.97 (m, 1H), 2.90-2.83 (m, 1H), 2.66-2.55 (m, 2H), 2.45-2.39 (m, 1H), 2.30-2.13 (m, 4H), 2.11-1.98 (m, 2H), 1.84-1.76 (m, 3H), 1.66-1.60 (m, 11H), 1.36-1.28 (m, 1H), 1.23-0.97 (m, 9H); LCMS (Method D): Rt=1.46 min, m/z=553.2 [M+H]⁺; HPLC (Method A): Rt=4.49 min, 98.62%; Chiral SFC (Method A): Rt=4.34 min, 100%.

### Example 2. N-Ethyl-5-fluoro-N-isopropyl-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamide (Compound No. 2)

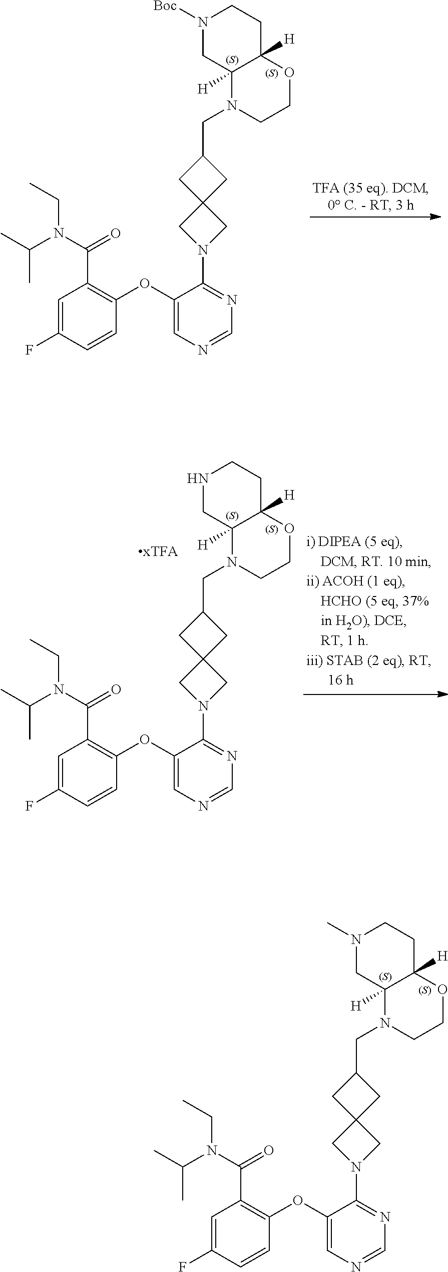

### Step 1. N-ethyl-5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide xTFA

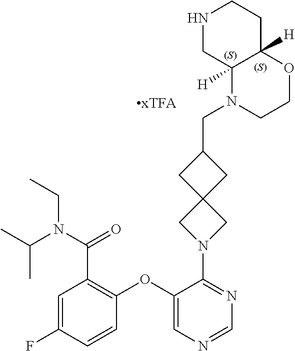

To a stirred solution of tert-butyl (4aS,8aS)-4-((2-(5-(2-(ethyl(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (0.47 g, 0.720 mmol) in dry DCM (20 mL) under a nitrogen atmosphere, TFA (1.941 mL, 25.2 mmol) was added at 0° C. The reaction was stirred at the same temperature for 15 min, then at RT for 3 h, and monitored by LCMS. After completion, the reaction was concentrated under reduced pressure to afford crude N-ethyl-5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide xTFA (0.5 g, quantitative yield) as a gum. This material was used without further purification. LCMS (Method B): Rt=1.34 min, m/z=553.4[M+H]⁺, 97.11%.

### Step 2. N-Ethyl-S-fluoro-N-isopropyl-2-((4-(6-(((4a,8aS)-6-methyloctahydro-4H-pyrido[4,3-b][1,4]oxazin-4-yl)methyl)-2-arsaspiro[3.3]heptan-2-yl)pyrimidin-5-yl)oxy)benzamide

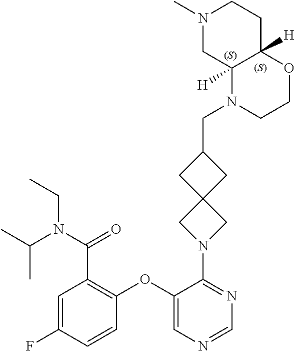

The reaction was performed following the same procedure in two batches (0.25 g×0.05 g).

To a stirred solution of N-ethyl-5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide-xTFA (0.25 g, 0.384 mmol) in dry DCM (15 mL), DIPEA (0.342 mL, 1.921 mmol) was added at RT under a nitrogen atmosphere. The reaction was stirred at the same temperature for 10 min, then was concentrated under reduced pressure. The crude was dissolved in DCE (15 mL), and formaldehyde (0.156 mL, 1.921 mmol, 37% in H₂O) and AcOH (0.023 g, 0.384 mmol) were added at RT. The reaction was stirred at the same temperature for 1 h. To this reaction mixture, sodium triacetoxyborohydride (0.163 g, 0.768 mmol) was added, and the mixture was stirred at RT for 16 h. The reaction was monitored by TLC (10% MeOH in DCM). The crude reaction mixtures from both batches were combined and concentrated under reduced pressure. The crude was purified by reverse phase chromatography using a SiliCycle SiliaSep C18 column on Biotage isolera one (Mobile phase A: 10 mM NH₄HCO₃ in water, B: ACN) to obtain N-ethyl-5-fluoro-N-isopropyl-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamide (0.16 g, 60.80% combined yield) as a solid. ¹H NMR (400 MHz, DMSO-d₆): δ 8.29-8.24 (m, 1H), 7.77-7.64 (m, 1H), 7.33-7.19 (m, 2H), 7.06-6.96 (m, 1H). 4.46-3.91 (m, 4H), 3.80-3.67 (m, 2H), 3.53-3.38 (m, 2H), 3.28-3.10 (m, 2H), 3.08-3.01 (m, 1H), 2.97-2.89 (m, 1H), 2.76-2.56 (m, 2H), 2.31-2.01 (m, 8H), 1.97-1.88 (m, 2H), 1.84-1.74 (m, 2H), 1.69-1.39 (m, 3H), 1.24-0.96 (m, 9H); LCMS (Method B): Rt=1.42 min, m/z=567.4 [M+H]⁺; HPLC (Method A): Rt=4.85 min, 99.57%; Chiral SFC (Method B): Rt=3.23 min, 99.18%.

### Example 3. 5-Fluoro-N,N-diisopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide (Compound No. 3)

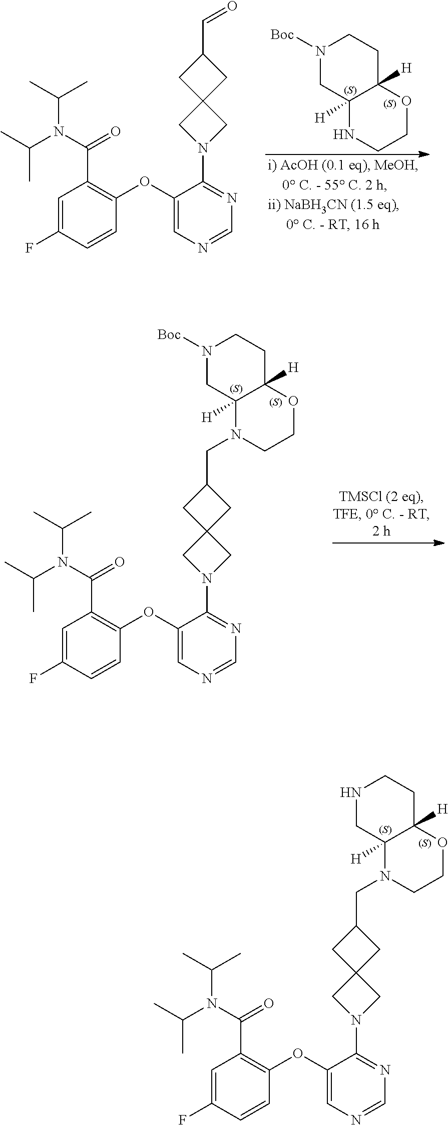

### Step 1. tert-Butyl (4aS,8aS)-4-((2-(S-(2-(diisopropylcarbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate

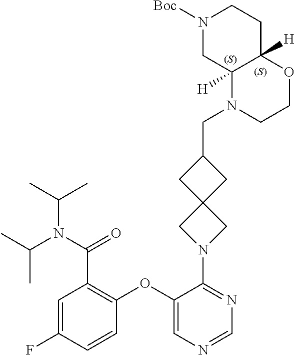

To a stirred solution of 5-fluoro-2-((4-(6-formyl-2-azaspiro[3.3]heptan-2-yl)pyrimidin-5-yl)oxy)-N,N-diisopropylbenzamide (0.4 g, 0.908 mmol) and tert-butyl (4aS,8aS)-hexahydro-2H-pyrido[4,3-b][1,4]oxazine-6(5H)-carboxylate (0.220 g, 0.908 mmol) in MeOH (5 mL), AcOH (5.45 mg, 0.091 mmol) was added at 0° C. under a nitrogen atmosphere. The reaction was stirred at 55° C. for 2 h. Sodium cyanoborohydride (0.086 g, 1.362 mmol) was then added at 0° C. The reaction was stirred at RT for 16 h, and monitored by TLC (100% EtOAc). The reaction was quenched with ice cold water (20 mL) and the mixture was extracted with EtOAc (2×50 mL). The combined organic layer was washed with brine solution (20 mL), dried over anhydrous sodium sulfate and filtered, and the filtrate was concentrated under reduced pressure. The crude product was purified by silica gel flash column chromatography using EtOAc in hexane (product eluted at 100% EtOAc) to obtain tert-butyl (4aS,8aS)-4-((2-(5-(2-(diisopropylcarbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (0.2 g, 33% yield) as a solid. LCMS (Method B): Rt=1.91 min, m/z=667.4 [M+H]⁺, 70.27%.

### Step 2. 5-Fluoro-N,N-diisopropyl-2-((4-(6-(((4aS,8aS)-octahydro-4H-pyrido[4.3-b][1,4]oxazin-4-yl)methyl)-2-azaspiro[3.3]heptan-2-yl)pyrimidin-5-yl)oxy)benzamide

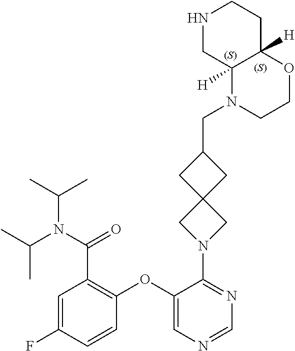

This compound was synthesized following the procedure described for the synthesis of Example 1, step 2, starting with 1 equivalent of tert-butyl (4aS,8aS)-4-((2-(5-(2-(diisopropylcarbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate. After completion, the reaction was concentrated under reduced pressure and the residue was triturated with EtOAc. The organic layer was decanted and the solid was dried under vacuum. The crude was purified by prep HPLC (Method C) to obtain 5-fluoro-N,N-diisopropyl-2-((4(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide as a solid.

Yield: 21.45%; ¹H NMR (400 MHz, DMSO-d₆): δ 8.26 (s, 1H), 7.73 (s, 1H), 7.25-7.18 (m, 2H), 7.03-6.97 (m, 1H), 4.25-4.15 (m, 1H), 4.14-4.04 (m, 2H), 4.02-3.94 (m, 1H), 3.72-3.63 (m, 2H), 3.57-3.47 (m, 2H), 3.18 (br dd, J=3.3, 12.3 Hz, 1H), 3.05-2.96 (m, 1H), 2.89-2.83 (m, 1H), 2.66-2.57 (m, 2H), 2.47-2.38 (m, 2H), 2.30-2.12 (m, 4H), 2.09-1.97 (m, 2H), 1.85-1.74 (m, 3H), 1.67-1.57 (m, 1H), 1.44 (d, J=6.8 Hz, 3H), 1.35 (d, J=6.6 Hz, 3H), 1.33-1.23 (m, 1H), 1.09 (d, J=6.5 Hz, 3H), 1.01-0.96 (m, 3H); LCMS (Method D): Rt=1.64 min, m/z=567.3 [M+H]⁺; HPLC (Method A): Rt=4.85 min, 98.72%; Chiral SFC (Method C): Rt=0.84 min, 100%.

### Example 4. N-Ethyl-5-fluoro-N-isopropyl-2-((4-(6-(((4aR,8aR)-octahydro-4H-pyrido[4,3-b][1,4]oxazin-4-yl)methyl)-2-azaspiro[3.3]heptan-2-yl)pyrimidin-S-yl)oxy)benzamide (Compound No. 4)

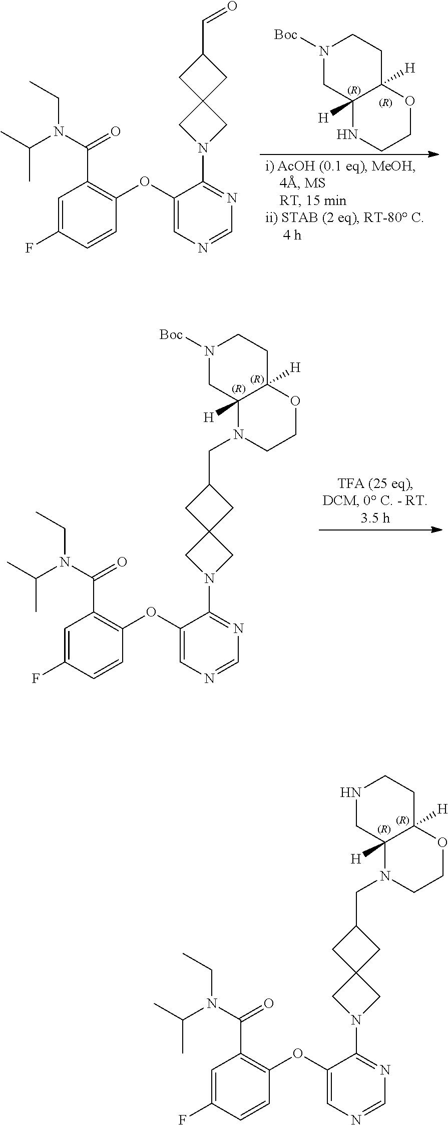

### Step 1. tert-Butyl (4aR,8aR)-4-((2-(5-(2-(ethyl(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate

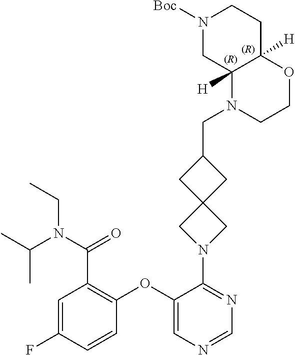

To a stirred solution of tert-butyl (4aR,8aR)-hexahydro-2H-pyrido[4,3-b][1,4]oxazine-6(5H)-carboxylate (0.16 g, 0.660 mmol) and N-ethyl-5-fluoro-2-((4-(6-formyl-2-azaspiro[3.3]heptan-2-yl)pyrimidin-5-yl)oxy)-N-isopropylbenzamide (0.282 g, 0.660 mmol) in dry MeOH (15 mL), AcOH (0.038 mL, 0.660 mmol) and 4 Å molecular sieves were added at RT under a nitrogen atmosphere. The reaction was stirred at the same temperature for 15 min. NaBH₃CN (0.083 g, 1.321 mmol) was then added at RT, and the reaction was stirred at RT for 10 min, then at 80° C. for 4 h. The reaction progress was monitored by TLC (10% MeOH in DCM). After completion, the reaction was filtered through a Celite® pad, and the pad was washed with MeOH (2×30 mL). The filtrate was concentrated under reduced pressure. The crude product was purified by reverse phase chromatography using a SiliCycle SiliaSep C18 column (120 g) on Biotage isolera one (Mobile phase A: 10 mM NH₄HCO₃ in water, B: ACN) to afford tert-butyl (4aR,8aR)-4-((2-(5-(2-(ethyl(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (0.191 g, 27.0% yield) as a solid. LCMS (Method D): Rt=2.03 min, m/z=653.2 [M+H]⁺, %91.03%.

### Step 2. N-Ethyl-5-fluoro-N-isopropyl-2-((4-(6-(((4aR,8aR)-octahydro-4H-pyrido[4,3-b][1,4]oxazin-4-yl)methyl)-2-azaspiro[3.3]heptan-2-yl)pyrimidin-5-yl)oxy)benzamide

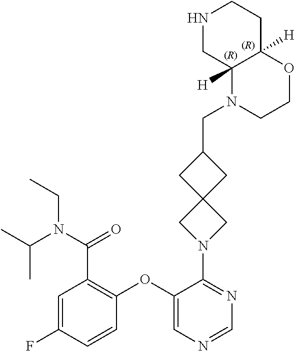

This compound was synthesized following the procedure described for the synthesis of Example 2, step 1, starting with 1 equivalent of tert-butyl (4aR,8aR)-4-((2-(5-(2-(ethyl(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate, except that 25 equivalents of TFA was used. After completion, the reaction was concentrated under reduced pressure. The crude material was purified Prep HPLC (Method D) to afford N-ethyl-5-fluoro-N-isopropyl-2-((4-(6-(((4aR,8aR)-octahydro-4H-pyrido[4,3-b][1,4]oxazin-4-yl)methyl)-2-azaspiro[3.3]heptan-2-yl)pyrimidin-5-yl)oxy)benzamide as a solid.

Yield: 63.7%; ¹H NMR (400 MHz, DMSO-d₆): δ 8.29-8.24 (m, 1H), 7.75-7.66 (m, 1H), 7.33-7.20 (m, 2H), 7.05-6.95 (m, 1H), 4.47-3.87 (m, 4H), 3.79-3.64 (m, 2H), 3.55-3.37 (m, 2H), 3.26-3.13 (m, 2H), 3.07-2.95 (m, 1H), 2.91-2.80 (m, 1H), 2.66-2.56 (m, 2H), 2.47-2.38 (m, 2H), 2.32-2.11 (m, 4H), 2.10-1.98 (m, 2H), 1.84-1.75 (m, 3H), 1.66-1.58 (m, 1H), 1.36-1.24 (m, 1H), 1.23-0.97 (m, 9H); LCMS (Method B): Rt=1.39 min, m/z=553.4[M+H]⁺; HPLC (Method A): Rt=6.62 min, 98.13%; Chiral SFC (Method D): Rt=3.82 min, 100%.

### Example 5. (±)-N-Ethyl-5-fluoro-N-isopropyl-2-((4-(6-(((trans)-octahydro-1H-pyrido[3,4-b][1,4]oxazin-1-yl)methyl)-2-azaspiro[3.3]heptan-2-yl)pyrimidin-5-yl)oxy)benzamide (Compound No. 5)

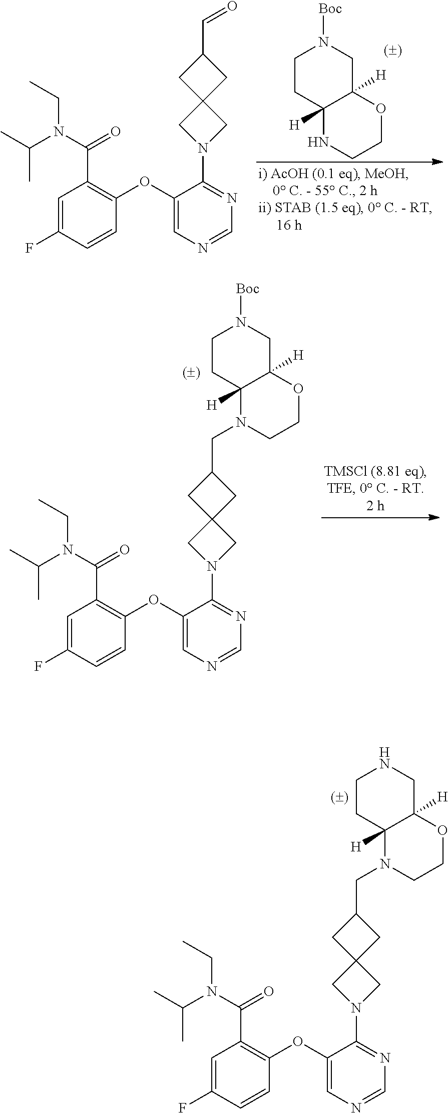 [SMILES: CCN(C(=O)c1cc(F)ccc1Oc1cncnc1N1CC2(CC(C=O)C2)C1)C(C)C ; CC(C)(C)OC(=O)N1CC[C@H]2NCCO[C@@H]2C1 ; CCN(C(=O)c1cc(F)ccc1Oc1cncnc1N1CC2(CC(CN3CCO[C@@H]4CN(C(=O)OC(C)(C)C)CC[C@H]43)C2)C1)C(C)C ; CCN(C(=O)c1cc(F)ccc1Oc1cncnc1N1CC2(CC(CN3CCO[C@@H]4CNCC[C@H]43)C2)C1)C(C)C]

### Step 1. (±)-tert-Butyl (4aR,8aR)-1-((2-(5-(2-(ethyl(isopropyl)carbamoyl)-4-fluorophenoxy)pyrimidin-4-yl)-2-azaspiro[3.3]heptan-6-yl)methyl)octahydro-6H-pyrido[3,4-b][1,4]oxazine-6-carboxylate

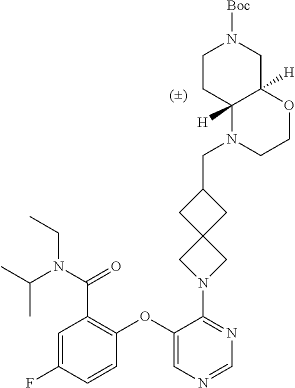 [SMILES: CCN(C(=O)c1cc(F)ccc1Oc1cncnc1N1CC2(CC(CN3CCO[C@@H]4CN(C(=O)OC(C)(C)C)CC[C@H]43)C2)C1)C(C)C]

This compound was synthesized following the procedure described for the synthesis of Example 3, step 1, starting with 1 equivalent of N-ethyl-5-fluoro-2-((4-(6-formyl-2-azaspiro[3.3]heptan-2-yl)pyrimidin-5-yl)oxy)-N-isopropylbenzamide and 1 equivalent of (±)-tert-butyl (trans)-octahydro-6H-pyrido[3,4-b][1,4]oxazine-6-carboxylate. After work-up, the filtrate was concentrated under reduced pressure to obtain crude (±)-tert-butyl (4aR,8aR)-1-((2-(5-(2-(ethyl(isopropyl)carbamoyl)-4-fluorophenoxy)pyrimidin-4-yl)-2-azaspiro[3.3]heptan-6-yl)methyl)octahydro-6H-pyrido[3,4-b][1,4]oxazine-6-carboxylate as a liquid. This material was used without further purification.

Yield: 54.6%; LCMS (Method B): Rt=1.37 min, m/z=653.4 [M+H]⁺, 72.03%.

### Step 2. (±)-N-Ethyl-S-fluoro-N-isopropyl-2-((4-(6-(((trans)-octahydro-1H-pyrido[3,4-b][1,4]oxazin-1-yl)methyl)-2-azaspiro[3.3]heptan-2-yl)pyrimidin-S-yl)oxy)benzamide

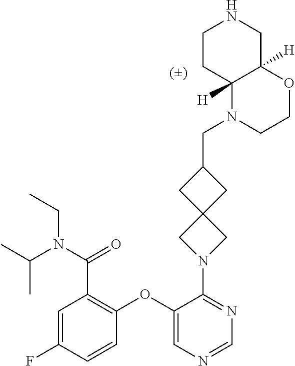 [SMILES: CCN(C(=O)c1cc(F)ccc1Oc1cncnc1N1CC2(CC(CN3CCO[C@@H]4CNCC[C@H]43)C2)C1)C(C)C]

This compound was synthesized following the procedure described for the synthesis of Example 1, step 2, starting with 1 equivalent of (±)-tert-butyl (4aR,8aR)-1-((2-(5-(2-(ethyl(isopropyl)carbamoyl)-4-fluorophenoxy)pyrimidin-4-yl)-2-azaspiro[3.3]heptan-6-yl)methyl)octahydro-6H-pyrido[3,4-b][1,4]oxazine-6-carboxylate, except that 8.81 equivalents of TMSCl was used. After completion, the reaction was concentrated under reduced pressure. The crude was purified by Prep HPLC (Method E). The pure fractions were concentrated under reduced pressure and lyophilized to afford (±)-N-ethyl-5-fluoro-N-isopropyl-2-((4-(6-(((trans)-octahydro-1H-pyrido[3,4-b][1,4]oxazin-1-yl)methyl)-2-azaspiro[3.3]heptan-2-yl)pyrimidin-5-yl)oxy)benzamide as a solid.

Yield: 38.4%; ¹H NMR (400 MHz, DMSO-d₆): δ 8.31-8.22 ((m, 1H), 7.78-7.67 (m, 1H), 7.32-7.19 (m, 2H), 7.05-6.93 (m, 11H), 4.46-3.90 (m, 4H), 3.80-3.64 (m, 2H), 3.55-3.37 (m, 2H), 3.26-3.11 (m, 2H), 3.04-2.94 (m, 1H), 2.90-2.79 (m, 2H), 2.70-2.64 (m, 2H), 2.43-2.35 (m, 2H), 2.31-2.11 (m, 5H), 2.08-1.98 (m, 1H), 1.95-1.85 (m, 11H), 1.84-1.75 (m, 3H), 1.26-0.95 (m, 9H); LCMS (Method D): Rt=1.56 min, m/z=553.1 [M+H]⁺; HPLC (Method A): Rt=4.57 min, 98.57%; Chiral SFC (Method E): Peak 1, Rt=3.44 min, 52.09%; Peak 2, Rt=5.41 min, 47.91%.

### Example 6. (k)-5-Fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N,N-diisopropylbenzamide (Compound No. 6)

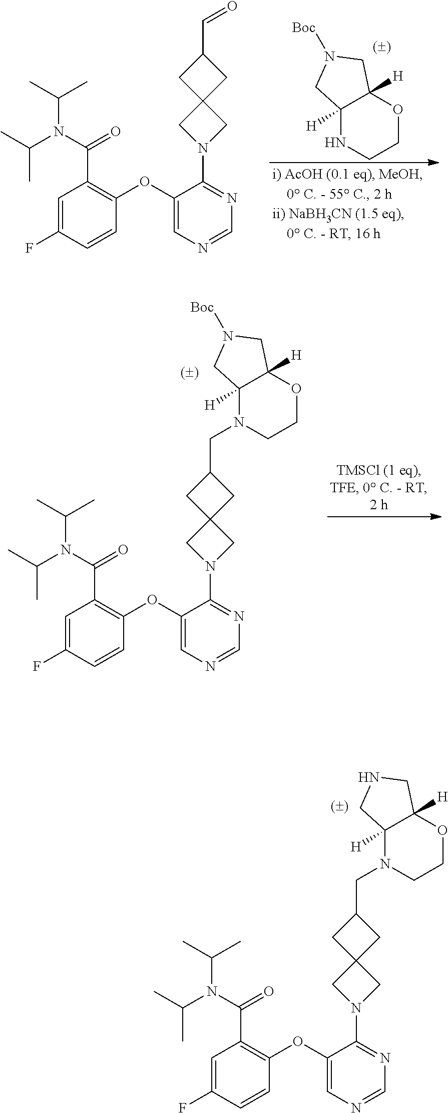 [SMILES: CC(C)N(C(=O)c1cc(F)ccc1Oc1cncnc1N1CC2(CC(C=O)C2)C1)C(C)C ; CC(C)(C)OC(=O)N1C[C@@H]2NCCO[C@H]2C1 ; CC(C)N(C(=O)c1cc(F)ccc1Oc1cncnc1N1CC2(CC(CN3CCO[C@H]4CN(C(=O)OC(C)(C)C)C[C@@H]43)C2)C1)C(C)C ; CC(C)N(C(=O)c1cc(F)ccc1Oc1cncnc1N1CC2(CC(CN3CCO[C@H]4CNC[C@@H]43)C2)C1)C(C)C]

### Step 1. (±)-tert-Butyl (4aS,7aS)-4-((2-(5-(2-(diisopropylcarbamoyl)-4-fluorophenoxy)pyrimidin-4-yl)-2-azaspiro[3.3]heptan-6-yl)methyl)hexahydropyrrolo[3,4-b][1,4]oxazine-6(2H)-carboxylate

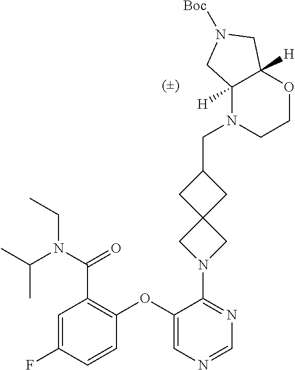 [SMILES: CCN(C(=O)c1cc(F)ccc1Oc1cncnc1N1CC2(CC(CN3CCO[C@H]4CN(C(=O)OC(C)(C)C)C[C@@H]43)C2)C1)C(C)C]

This compound was synthesized following the procedure described for the synthesis of Example 3, step 1, starting with 1 equivalent of 5-fluoro-2-((4-(6-formyl-2-azaspiro[3.3]heptan-2-yl)pyrimidin-5-yl)oxy)-N,N-diisopropylbenzamide and 1 equivalent of (±)-tert-butyl (trans)-hexahydropyrrolo[3,4-b][1,4]oxazine-6(2H)-carboxylate. After completion, the reaction was quenched with ice cold water and the mixture was extracted with DCM. The combined organic layer was washed with brine solution, dried over anhydrous sodium sulfate and filtered, and the filtrate was concentrated under reduced pressure. The crude product was purified by silica gel flash column chromatography using EtOAc in hexane (product eluted at 90% to 100% EtOAc) to obtain (f)-tert-butyl (4aS,7aS)-4-((2-(5-(2-(diisopropylcarbamoyl)-4-fluorophenoxy)pyrimidin-4-yl)-2-azaspiro[3.3]heptan-6-yl)methyl)hexahydropyrrolo[3,4-b][1,4]oxazine-6(21)-carboxylate (440 mg. 47.2% yield) as a solid. LCMS (Method B): Rt=2.00 min, m/z=653.5 [M+H]⁺, 95.40/o.

### Step 2. (±)-5-Fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N,N-diisopropylbenzamide

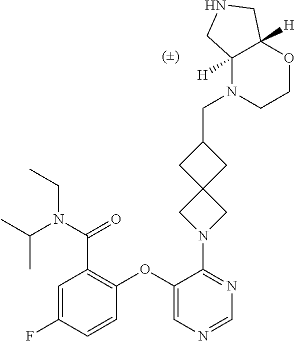 [SMILES: CCN(C(=O)c1cc(F)ccc1Oc1cncnc1N1CC2(CC(CN3CCO[C@H]4CNC[C@@H]43)C2)C1)C(C)C]

This compound was synthesized following the procedure described for the synthesis of Example 1, step 2, starting with 1 equivalent of (±)-tert-butyl (4aS,7aS)-4-((2-(5-(2-(diisopropylcarbamoyl)-4-fluorophenoxy)pyrimidin-4-yl)-2-azaspiro[3.3]heptan-6-yl)methyl)hexahydropyrrolo[3,4-b][1,4]oxazine-6(2H)-carboxylate, except that 1 equivalent of TMSCl was used. Ater completion, the reaction was concentrated under reduced pressure. The crude was purified by Prep HPLC (Method F) to obtain (±)-5-fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N,N-diisopropylbenzamide as a solid.

Yield: 37.3%; ¹H NMR (400 MHz, DMSO-d₆): δ 8.26 (s, 1H), 7.75-7.71 (m, 1H), 7.27-7.17 (m, 2H), 7.04-6.95 (m, 1H), 4.24-4.16 (m, 1H), 4.13-4.02 (m, 2H), 4.01-3.92 (m, 1H), 3.86-3.77 (m, 1H), 3.73-3.64 (m, 1H), 3.59-3.42 (m, 4H), 3.04-2.96 (m, 1H), 2.93-2.86 (m, 1H), 2.75-2.70 (m, 1H), 2.61-2.56 ((m, 1H), 2.45-2.35 (m, 2H), 2.30-2.17 (m, 3H), 2.17-2.08 (m, 1H), 2.04-1.92 (m, 2H), 1.87-1.74 (m, 2H), 1.44 (d, J=6.6 Hz, 3H), 1.35 (d, J=6.8 Hz, 3H), 1.09 (d, J=6.6 Hz, 3H), 0.99 (br d, J=6.5 Hz, 3H); LCMS (Method D): Rt=1.61 min, m/z=553.2 [M+H]⁺; HPLC (Method A): Rt=4.60 min, 99.08%.

### Example 7. (E1)-5-Fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N,N-diisopropylbenzamide (Compound No. 7)

### Example 8. (E2)-5-Fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N,N-diisopropylbenzamide (Compound No. 8)

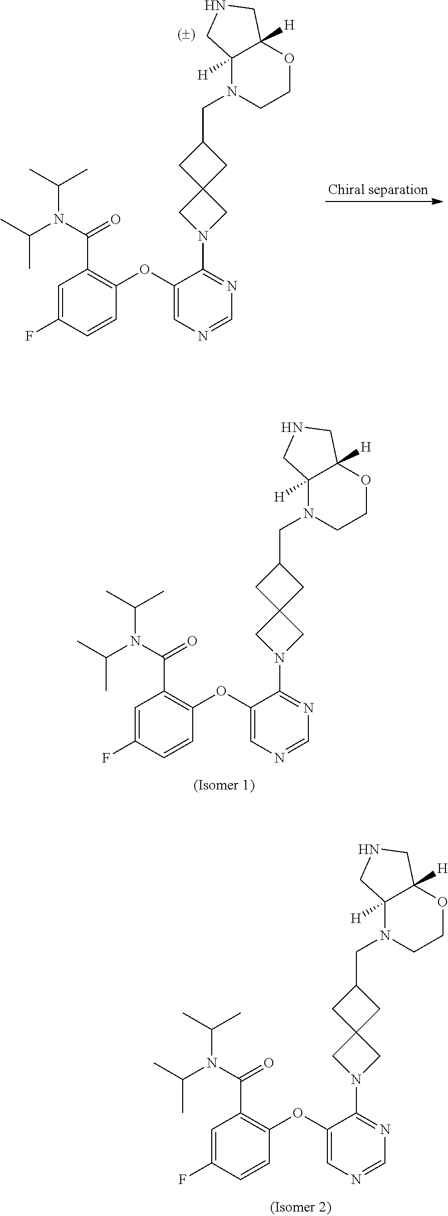 [SMILES: CC(C)N(C(=O)c1cc(F)ccc1Oc1cncnc1N1CC2(CC(CN3CCO[C@H]4CNC[C@@H]43)C2)C1)C(C)C ; CC(C)N(C(=O)c1cc(F)ccc1Oc1cncnc1N1CC2(CC(CN3CCO[C@H]4CNC[C@@H]43)C2)C1)C(C)C ; CC(C)N(C(=O)c1cc(F)ccc1Oc1cncnc1N1CC2(CC(CN3CCO[C@H]4CNC[C@@H]43)C2)C1)C(C)C]

(±)-5-Fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N,N-diisopropylbenzamide (74 mg, 0.113 mmol) was purified by chiral Prep SFC (Method F) to obtain both isomers.

Isomer 1: (E1)-5-Fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N,N-diisopropylbenzamide (22.53 mg, 35.6% yield) as a solid. ¹H NMR (400 MHz, DMSO-d₆): δ 8.26 (s, 1H), 7.74-7.68 (m, 1H), 7.25-7.19 (m, 2H), 7.05-6.96 (m, 1H), 4.26-4.16 (m, 1H), 4.14-4.04 (m, 2H), 4.03-3.93 (m, 1H), 3.85-3.76 ((m, 1H), 3.73-3.62 (m, 1H), 3.60-3.43 (m, 4H), 3.03-2.95 (m, 1H), 2.94-2.83 (m, 1H), 2.76-2.69 (m, 1H), 2.62-2.556 (m, 1H), 2.47-2.35 (m, 2H), 2.30-2.18 (m, 3H), 2.16-1.92 (m, 3H), 1.86-1.76 (m, 2H), 1.45 (d, J=6.6 Hz, 3H), 1.35 (d, J=6.8 Hz, 3H), 1.09 (d, J=6.5 Hz, 3H), 1.00 (br d, J=6.6 Hz, 3H); LCMS (Method D): Rt=1.52 min, m/z=553.6 [M+H]⁺; HPLC (Method A): Rt=4.62 min, 99.00%; Chiral SFC (Method H): Rt=7.00 min, 100%.

Isomer 2: (E2)-5-Fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N,N-diisopropylbenzamide (22.71 mg, 35% yield) as a solid. ¹H NMR (400 MHz, DMSO-d₆): δ 8.26 (s, 1N), 7.75-7.68 (m, 1H), 7.27-7.17 (m, 2H), 7.06-6.96 (m, 1H), 4.26-4.17 (m, 1H), 4.14-4.04 (m, 2H), 4.03-3.94 (m, 1H), 3.85-3.76 (m, 1H), 3.73-3.61 (m, 1H), 3.61-3.47 (m, 4H), 3.45-3.39 (m, 1H), 3.03-2.87 (m, 1H), 2.86-2.76 (m, 1H), 2.76-2.68 (m, 1H), 2.65-2.57 (m, 1H), 2.44-2.36 (m, 1H), 2.30-2.18 (m, 3H), 2.17-2.07 (m, 1H), 2.06-1.93 (m, 2H), 1.89-1.73 (m, 2H), 1.44 (d, J=6.6 Hz, 3H), 1.35 (br d, J=6.5 Hz, 3H), 1.11-1.05 (m, 314), 1.00 (br d, J=6.4 Hz, 3H); LCMS (Method D): Rt=1.49 min, m/z=553.3 [M+H]⁺; HPLC (Method A): Rt=4.56 min, 96.48%; Chiral SFC (Method H): Rt=8.42 min, 96.86%.

### Example 9. (E1)-N-Ethyl-5-fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N-isopropylbenzamide (Compound No. 9)

### Example 10. (E2)-N-Ethyl-5-fluoro-2-((4-(6-(((rans)-hexahydropyrrolo[3,4-b][1,4]oxazin-4(4aH)-yl)methyl)-2-azaspiro[3.3]heptan-2-yl)pyrimidin-5-yl)oxy)-N-isopropylbenzamide (Compound No. 10)

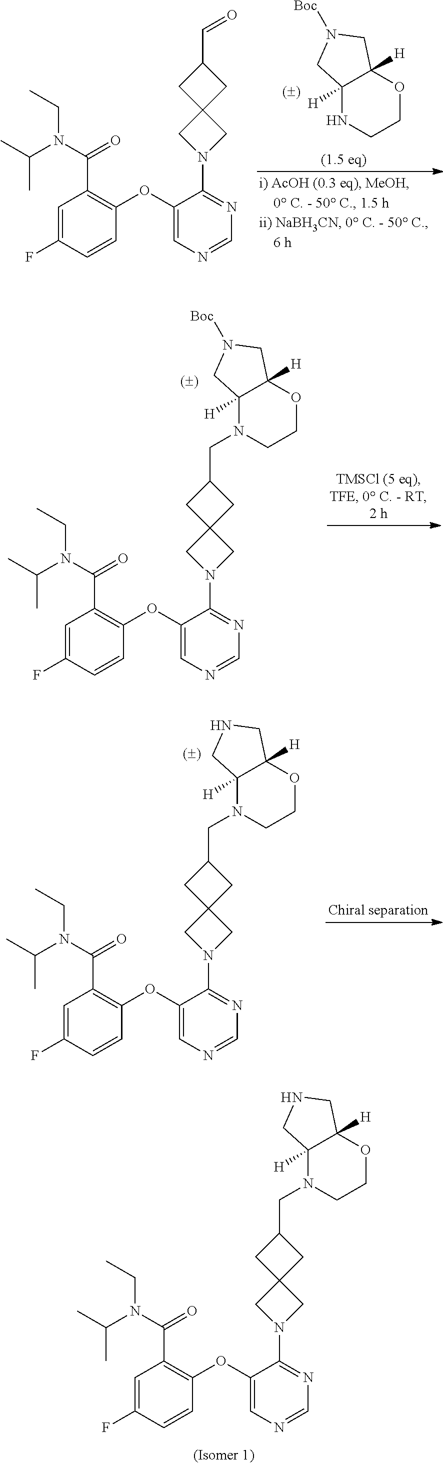 [SMILES: CCN(C(=O)c1cc(F)ccc1Oc1cncnc1N1CC2(CC(C=O)C2)C1)C(C)C ; CC(C)(C)OC(=O)N1C[C@@H]2NCCO[C@H]2C1 ; CCN(C(=O)c1cc(F)ccc1Oc1cncnc1N1CC2(CC(CN3CCO[C@H]4CN(C(=O)OC(C)(C)C)C[C@@H]43)C2)C1)C(C)C ; CCN(C(=O)c1cc(F)ccc1Oc1cncnc1N1CC2(CC(CN3CCO[C@H]4CNC[C@@H]43)C2)C1)C(C)C ; CCN(C(=O)c1cc(F)ccc1Oc1cncnc1N1CC2(CC(CN3CCO[C@H]4CNC[C@@H]43)C2)C1)C(C)C]

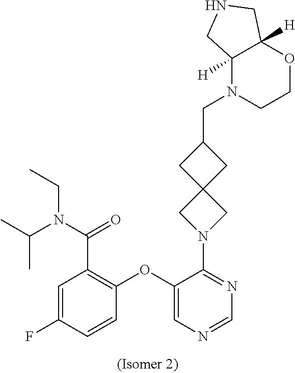 [SMILES: CCN(C(=O)c1cc(F)ccc1Oc1cncnc1N1CC2(CC(CN3CCO[C@H]4CNC[C@@H]43)C2)C1)C(C)C]

### Step 1. (±)-tert-Butyl (4aS,7aS)-4-((2-(5-(2-(ethyl(isopropyl)carbamoyl)-4-fluorophenoxy)pyrimidin-4-yl)-2-azaspiro[3.3]heptan-6-yl)methyl)hexahydropyrrolo[3,4-b][1,4]oxazine-6(2H)-carboxylate

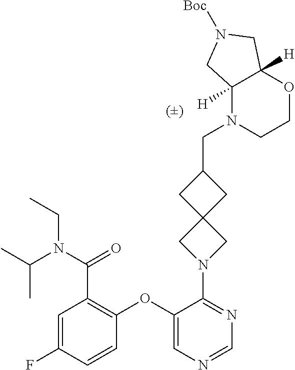 [SMILES: CCN(C(=O)c1cc(F)ccc1Oc1cncnc1N1CC2(CC(CN3CCO[C@H]4CN(C(=O)OC(C)(C)C)C[C@@H]43)C2)C1)C(C)C]

In a dried, 50 mL two neck round bottom flask, N-ethyl-5-fluoro-2-((4-(6-formyl-2-azaspiro[3.3]heptan-2-yl)pyrimidin-5-yl)oxy)-N-isopropylbenzamide (250 mg, 0.586 mmol) and (±)-tert-butyl (trans)-hexahydropyrrolo[34-b][1,4]oxazine-6(2H)-carboxylate (201 mg, 0.879 mmol) in MeOH (10 mL), AcOH (10.07 μL, 0.176 mmol) was added at 0° C. The reaction was stirred at 50° C. for 1.5 h. To this reaction mixture, sodium cyanoborohydride (73.7 mg, 1.172 mmol) was added at 0° C. The reaction was stirred at 50° C. for 6 h, and monitored by LCMS and TLC (100% EtOAc). After completion, the reaction was concentrated under reduced pressure. The residue was dissolved in DCM (50 mL) and the mixture was washed with water (2×10 mL) then with brine solution (10 mL). The organic layer was dried over anhydrous Na₂SO₄ and filtered, and the filtrate was concentrated under reduced pressure. The crude compound was triturated with hexane (2×10 mL) and the organic layer was decanted. The residue was dried under vacuum to obtain crude (±)-tert-butyl (4aS,7aS)-4-((2-(5-(2-(ethyl(isopropyl)carbamoyl)-4-fluorophenoxy)pyrimidin-4-yl)-2-azaspiro[3.3]heptan-6-yl)methyl)hexahydropyrrolo[3,4-b][1,4]oxazine-6(2H)-carboxylate (250 mg, 65% yield) as a gummy solid. This material was used without further purification. LCMS (Method D): Rt=2.07 min, m/z=639.3 [M+H]⁺, 97.37%.

### Step 2. (±)-N-Ethyl-5-fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N-isopropylbenzamide

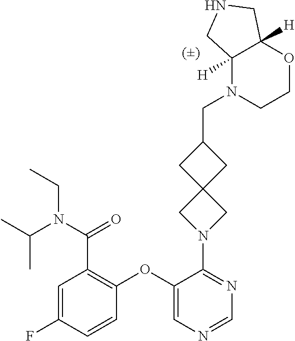 [SMILES: CCN(C(=O)c1cc(F)ccc1Oc1cncnc1N1CC2(CC(CN3CCO[C@H]4CNC[C@@H]43)C2)C1)C(C)C]

This compound was synthesized following the procedure described for the synthesis of Example 1, step 2, starting with 1 equivalent of (±)-tert-butyl (4aS,7aS)-4-((2-(5-(2-(ethyl(isopropyl)carbamoyl)-4-fluorophenoxy)pyrimidin-4-yl)-2-azaspiro[3.3]heptan-6-yl)methyl)hexahydropyrrolo[3,4-b][1,4]oxazine-6(2H)-carboxylate, except that 5 equivalents of TMSCl was used. After completion, the reaction was concentrated under reduced pressure. The crude compound was purified by Prep HPLC (Method G) to obtain (±)-N-ethyl-5-fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N-isopropylbenzamide as a gum.

Yield: 52%; LCMS (Method D): Rt=1.52 min, m/z=539.2 [M+H]⁺, 99.75%.

### Step 3. (E1)-N-Ethyl-5-fluoro-2-((4-(6-(((trans)-hexahydropyrrolo[3.4-b][1,4]oxazin-4(4aH)-yl)methyl)-2-azaspiro[3.3]heptan-2-yl)pyrimidin-5-yl)oxy)-N-isopropylbenzamide and (E2)-N-ethyl-5-fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N-isopropylbenzamide

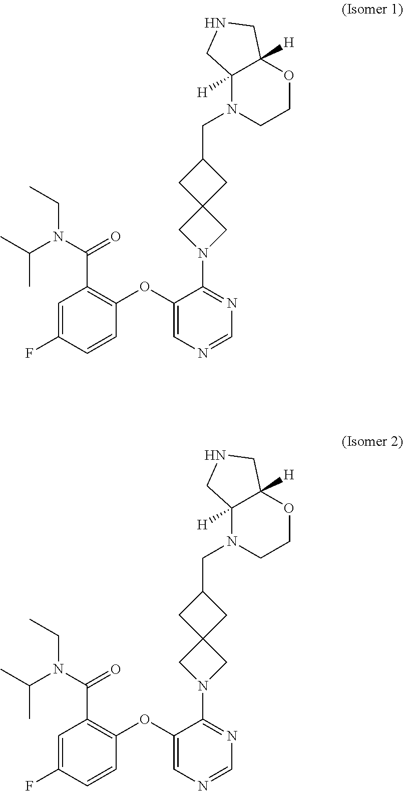 [SMILES: CCN(C(=O)c1cc(F)ccc1Oc1cncnc1N1CC2(CC(CN3CCO[C@H]4CNC[C@@H]43)C2)C1)C(C)C ; CCN(C(=O)c1cc(F)ccc1Oc1cncnc1N1CC2(CC(CN3CCO[C@H]4CNC[C@@H]43)C2)C1)C(C)C]

(±)-N-Ethyl-5-fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N-isopropylbenzamide (110 mg, 0.204 mmol) was purified by chiral Prep SFC (Method A) to obtain both isomers. Subsequently, isomer 2 was re-purified by chiral Prep SFC (Method A).

Isomer 1: (E1)-N-Ethyl-5-fluoro-2-((4-(6-((trans)-hexahydropyrrolo[3.4-b][1,4]oxazin-4(4aH)-yl)methyl)-2-azaspiro[3.3]heptan-2-yl)pyrimidin-5-yl)oxy)-N-isopropylbenzamide (28.11 g, 25.5% yield) as a solid. ¹H NMR (400 MHz, DMSO-d₆): δ 8.29-8.24 (m, 1H), 7.77-7.65 (m, 1H), 7.34-7.20 (m, 2H), 7.06-6.94 (m, 1H), 4.49-3.90 (m, 4H), 3.87-3.71 (m, 2H), 3.63-3.49 (m, 2H), 3.45-3.38 (m, 1H), 3.27-3.10 (m, 2H), 3.04-2.87 (m, 2H), 2.85-2.69 (m, 1H), 2.62-2.54 ((m, 1H), 2.47-2.35 (m, 1H), 2.31-2.18 (m, 3H), 2.17-2.07 (m, 1H), 2.06-1.91 (m, 2H), 1.90-1.73 (m, 2H), 1.28-0.96 (m, 9H), one proton merged with solvent peaks; LCMS (Method D): Rt=1.52 min, m/z=539.2 [M+H]⁺; HPLC (Method A): Rt=4.27 min, 99.59%; Chiral SFC (Method 3): Rt=4.35 min, 100%.

Isomer 2: (E2)-N-Ethyl-5-fluoro-2-((4-(6-(((trans)-hexahydropyrrolo[3,4-b][1,4]oxazin-4(4aH)-yl)methyl)-2-azaspiro[3.3]heptan-2-yl)pyrimidin-5-yl)oxy)-N-isopropylbenzamide (14.28 g, 13.37% yield) as a solid. ¹H NMR (400 MHz, DMSO-d₆): δ 8.32-8.23 (m, 1H), 7.78-7.65 (m, 11H), 7.33-7.21 (m, 2H), 7.05-6.94 (m, 1H), 4.51-3.88 (m, 4H), 3.85-3.69 (m, 2H), 3.65-3.48 (m, 2H), 3.47-3.39 (m, 1H), 3.26-3.12 (in. 2H), 3.08-2.92 (m, 2H), 2.76-2.70 (m, 1H), 2.65-2.56 ((m, 1H), 2.43-2.36 ((m, 1H), 2.31-2.17 (m, 3H), 2.16-2.08 (m, 1H), 2.06-1.92 (m, 2H), 1.88-1.70 (m, 2H), 1.24-0.96 (m, 9H), one proton merged with solvent peaks; LCMS (Method D): Rt=1.49 min, m/z 539.2 [M+H]⁺; HPLC (Method A): Rt=4.19 min, 98.10%; Chiral SFC (Method J): Rt=5.22 min, 99.02%.

### Example 11. (R)-2-((4-(6-((2-(Aminomethyl)pyrrolidin-1-yl)methyl)-2-azaspiro[3.3]heptan-2-yl)pyrimidin-5-yl)oxy)-N-ethyl-S-fluoro-N-isopropylbenzamide (Compound No. 11)

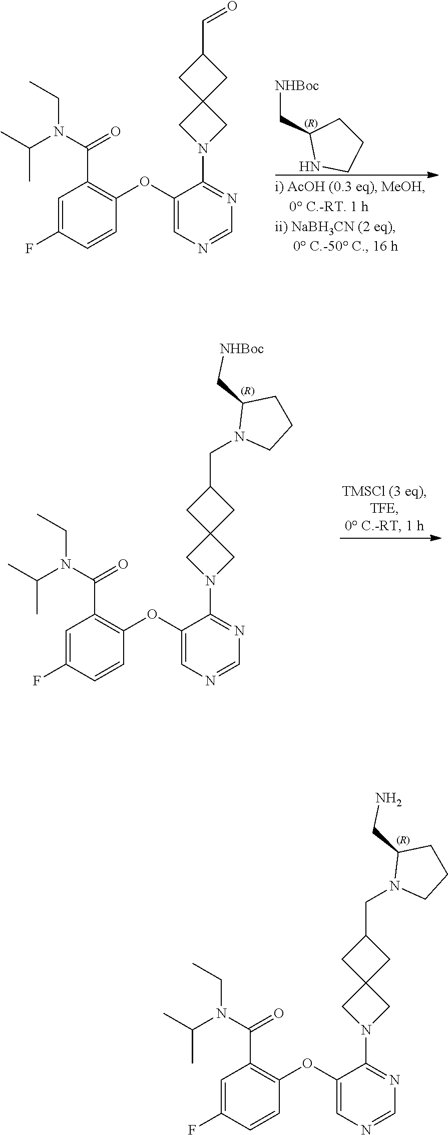

### Step 1. tert-Butyl (R)-((1-((2-(S-(2-ethyl(isopropyl)carbamoyl)-4-fluorophenoxy)pyrimidin-4-yl)-2-azaspiro[3.3]heptan-6-yl)methyl)pyrimidin-2-yl)methyl)carbamate

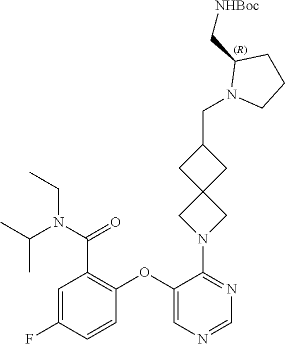

This compound was synthesized following the procedure described for the synthesis of Example 1, step 1, starting with 1 equivalent of N-ethyl-5-fluoro-2-((4-(6-formyl-2-azaspiro[3.3]heptan-2-yl)pyrimidin-5-yl)oxy)-N-isopropylbenzamide and 1 equivalent of tert-butyl(R)-(pyrrolidin-2-ylmethyl)carbamate, except that 0.3 equivalent of AcOH and 2 equivalents of NaBH₃CN were used. The reaction was concentrated and under reduced pressure. The residue was dissolved in DCM, washed with sodium bicarbonate solution and brine, dried over anhydrous sodium sulfate and filtered. The filtrate was concentrated under reduced pressure to afford crude tert-butyl (R)-((1-((2-(5-(2-(ethyl(isopropyl)carbamoyl)-4-fluorophenoxy)pyrimidin-4-yl)-2-azaspiro[3.3]heptan-6-yl)methyl)pyrrolidin-2-yl)methyl)carbamate. This material was used without further purification.

Yield: 37.6%; LCMS-ELSD (Method D): Rt=2.15 min, m/z=611.3 [M+H]⁺, 77.21%.

### Step 2. (R)-2-((4-(6-((2-(Aminomethyl)pyrolidin-1-yl)methyl)-2-azaspiro[3.3]heptan-2-yl)pyrimidin-5-yl)oxy)-N-ethyl-5-fluoro-N-isopropylbenzamide

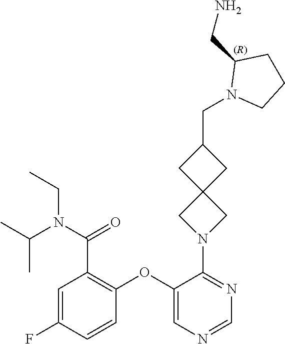

This compound was synthesized following the procedure described for the synthesis of Example 1, step 2, starting with 1 equivalent of tert-butyl (R)-((1-((2-(5-(2-(ethyl(isopropyl)carbamoyl)-4-fluorophenoxy)pyrimidin-4-yl)-2-azaspiro[3.3]heptan-6-yl)methyl)pyrrolidin-2-yl)methyl)carbamate, except that 3 equivalents of TMSCl was used. After completion, the reaction was concentrated under reduced pressure, and the crude was purified by Prep HPLC (Method H) to obtain (R)-2-((4-(6-((2-(aminomethyl)pyrrolidin-1-yl)methyl)-2-azaspiro[3.3]heptan-2-yl)pyrimidin-5-yl)oxy)-N-ethyl-5-fluoro-N-isopropylbenzamide as a semi-solid.

Yield: 16.92%; ¹H NMR (400 MHz, DMSO-d₆): δ 8.29-8.24 (m, 1H), 7.76-7.66 (m, 1H), 7.33-7.20 (m, 2H), 7.07-6.96 (m, 1H), 4.48-3.94 (m, 4H), 3.75 (Sep, J=6.7 Hz, 1H), 3.52-3.47 (m, 1H), 3.45-3.38 (m, 2H), 3.27-3.11 (m, 2H), 2.97-2.89 (m, 1H), 2.67-2.59 (m, 1H), 2.48-2.39 (m, 1H), 2.30-2.11 (m, 5H), 2.08-2.00 (n, 1H), 1.85-1.66 (m, 3H), 1.65-1.48 (m, 3H), 1.24-0.97 (m, 9H); LCMS (Method D): Rt=1.50 min, m/z=511.2 [M+H]⁺; HPLC (Method A): Rt=4.29 min, 98.58%; Chiral SFC (Method O): Rt=4.09 min, 100%.

### Example 12. (S)-2-((4-(6-((2-(Aminomethyl)pyrrolidin-1-yl)methyl)-2-azaspiro[3.3]heptan-2-yl)pyrimidin-5-yl)oxy)-N-ethyl-5-fluoro-N-isopropylbenzamide (Compound No. 12)

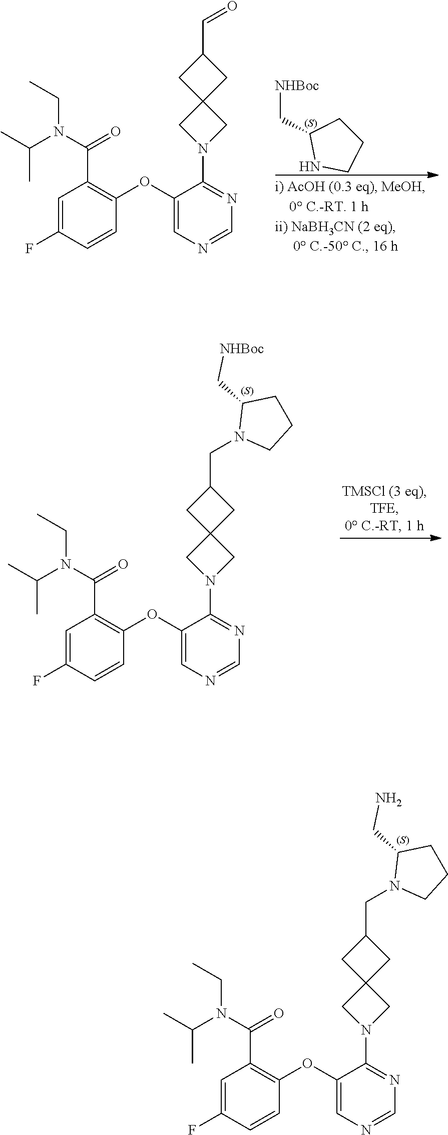

### Step 1. tert-Butyl (S)-((1-((2-(5-(2-(ethyl(isopopyl)carbamoyl)-4-fluorophenoxy)pyrimidin-4-yl)-2-azaspiro[3.3]heptan-6-yl)methyl)pyrrolidin-2-yl)methyl)carbamate

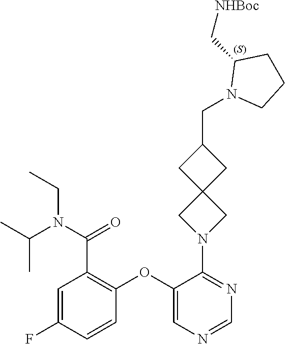

This compound was synthesized following the procedure described for the synthesis of Example 1, step 1, starting with 1 equivalent of N-ethyl-5-fluoro-2-((4-(6-formyl-2-azaspiro[3.3]heptan-2-yl)pyrimidin-5-yl)oxy)-N-isopropylbenzamid and 1 equivalents of tert-butyl (S)-(pyrrolidin-2-ylmethyl)carbamate, except that 0.3 equivalent of AcOH and 2 equivalents of NaBH₃CN were used.

The reaction was concentrated and under reduced pressure. The residue was dissolved in DCM, washed with sodium bicarbonate solution and brine, dried over anhydrous sodium sulfate and filtered. The filtrate was concentrated under reduced pressure to afford crude tert-butyl (S)-((1-((2-(S-(2-(ethyl(isopropyl)carbamoyl)-4-fluorophenoxy)pyrimidin-4-yl)-2-azaspiro[3.3]heptan-6-yl)methyl)pyrrolidin-2-yl)methyl)carbamate. This material was used without further purification.

Yield: 12.90%; LCMS-ELSD (Method D): Rt=2.19 min, m/z=611.2 [M+H]⁺, 84.00%.

### Step 2. (S)-2-((4-(6-((2-(Aminomethyl)pyrrolidin-1-yl)methyl)-2-azaspiro[3.3]heptan-2-yl)pyrimidin-S-yl)oxy)-N-ethyl-5-fluoro-N-isopropylbenzamide

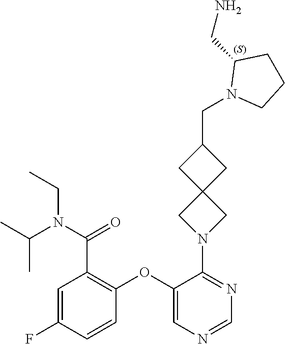

This compound was synthesized following the procedure described for the synthesis of Example 1, step 2, starting with 1 equivalent of tert-butyl (S)-((1-((2-(5-(2-(ethyl(isopropyl)carbamoyl)-4-fluorophenoxy)pyrimidin-4-yl)-2-azaspiro[3.3]heptan-6-yl)methyl)pyrrolidin-2-yl)methyl)carbamate, except that 3 equivalents of TMSCl was used. After completion, the reaction was concentrated under reduced pressure, and the crude was purified by prep HPLC (Method T) to obtain (S)-2-((4-(6-((2-(aminomethyl)pyrrolidin-1-yl)methyl)-2-azaspiro[3.3]heptan-2-yl)pyrimidin-5-yl)oxy)-N-ethyl-5-fluoro-N-isopropylbenzamide as a semi-solid.

Yield: 36.8%; ¹H NMR (400 MHz, DMSO-d₆): δ 8.31-8.24 ((m, 1H), 7.78-7.65 (m, 1H), 7.33-7.20 (m, 2H), 7.06-6.94 (m, 1H), 4.47-3.94 (m, 4H), 3.83-3.71 (m, 1H), 3.46-3.38 (m, 2H), 3.27-3.12 ((m, 2H), 2.98-2.90 (m, 1H), 2.65-2.59 (m, 2H), 2.31-2.12 (m, 5H), 2.10-2.01 (m, 1H), 1.85-1.67 (m, 3H), 1.65-1.51 (m, 3H), 1.22-0.97 (m, 9H), one protons merged with solvent peaks; LCMS (Method D): Rt=1.64 min, m/z=511.2 [M+H]⁺; HPLC (Method A): Rt=4.26 min, 98.99%; Chiral SFC (Method Q): Rt=1.85 min, 95.55%.

### Example 13. ((3R,5R)-3,5-Dimethylmorpholino)(5-fluoro-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)phenyl)methanone (Compound No. 13)

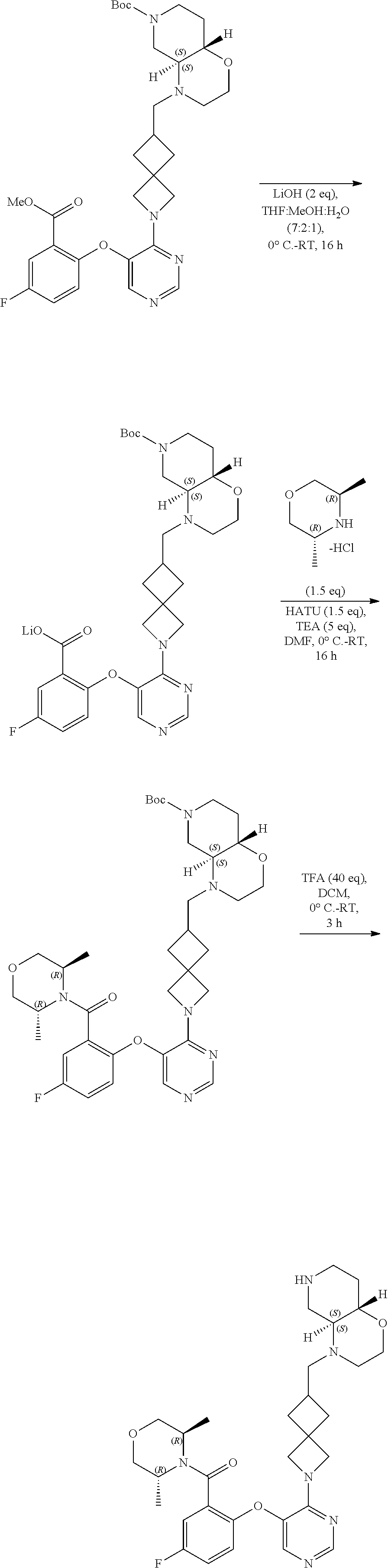

### Step 1. Lithium 2-((4-(6-(((4aS,8aS)-6-(tert-butoxycarbonyl)octahydro-4H-pyrido[4,3-b][1,4]oxazin-4-yl)methyl)-2-azaspiro[3.3]heptan-2-yl)pyrimidin-S-yl)oxy)-5-fluorobenzoate

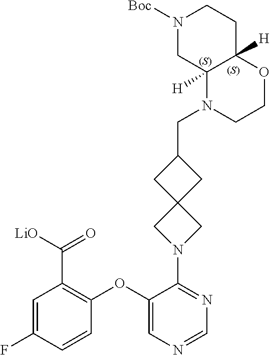

In a 25 mL round bottom flask, tert-butyl (4aS,8aS)-4-((2-(5-(4-fluoro-2-(methoxycarbonyl)phenoxy)pyrimidin-4-yl)-2-azaspiro[3.3]heptan-6-yl)methyl)hexahydro-2H-pyrido[4,3-b][1,4]oxazine-6(5H)-carboxylate (0.2 g, 0.335 mmol) was dissolved in 7:2 THF:MeOH (10 mL:2.86 mL). To this, a solution of LiOH (0.016 g, 0.669 mmol) in water (1.4 mL) was added. The reaction was stirred at RT for 16 h, and monitored by TLC (10/a MeOH in DCM). After completion, the reaction was concentrated under reduced pressure to afford crude lithium 2-((4-(6-(((4aS,8aS)-6-(tert-butoxycarbonyl)octahydro-4H-pyrido[4,3-b][1,4]oxazin-4-yl)methyl)-2-azaspiro[3.3]heptan-2-yl)pyrimidin-5-yl)oxy)-5-fluorobenzoate (0.21 g, 57.1% yield) as a solid. This material was used without further purification. LCMS (Method D): Rt=1.33 min, m/z=584.1 [M+H]⁺, 53.62%.

### Step 2. tert-Butyl (4aS,8aS)-4-((2-(5-(2-((3R,5R)-3,5-dimethylmorpholine-4-carbonyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate

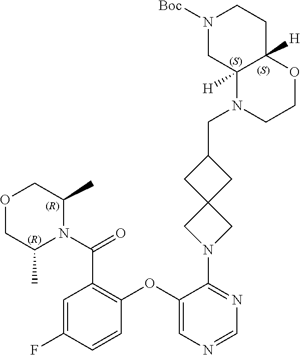

In a 50 mL round bottom flask under a nitrogen atmosphere, lithium 2-((4-(6-(((4aS,8aS)-6-(tert-butoxycarbonyl)octahydro-4H-pyrido[4,3-b][1,4]oxazin-4-yl)methyl)-2-azaspiro[3.3]heptan-2-yl)pyrimidin-5-yl)oxy)-5-fluorobenzoate (0.2 g, 0.343 mmol) was dissolved in DMF (10 mL). To this solution, Et₃N (0.239 mL, 1.713 mmol), HATU (0.195 g, 0.514 mmol) and (3R,5R)-3,5-dimethylmorpholine hydrochloride (0.052 g, 0.343 mmol) were added at RT. The reaction was stirred at RT for 16 h, and monitored by TLC (10% MeOH in DCM). The reaction was diluted with DCM (40 mL) and washed with water (2×30 mL). The combined organic layer was dried over sodium sulfate and filtered, and the filtrate was concentrated under reduced pressure. The residue was purified by silica gel flash column chromatography using MeOH in DCM (product eluted at 5% MeOH in DCM) to obtain tert-butyl (4aS,8aS)-4-((2-(5-(2-((3R,5R)-3,5-dimethylmorpholine-4-carbonyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (0.150 g, 26.3% yield) as a semi-solid. LCMS (Method D): Rt=1.94 min, m/z=681.2 [M+H]⁺, 40.88%.

### Step 3. ((3R,5R)-3,5-Dimethylmorpholino)(5-fluoro-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)phenyl)methanone

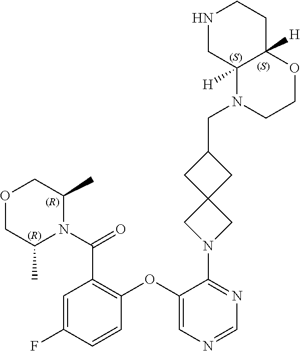

This compound was synthesized following the procedure described for the synthesis of Example 2, step 1, starting with 1 equivalent of tert-butyl (4aS,8aS)-4-((2-(5-(2-((3R,5R)-3,5-dimethylmorpholine-4-carbonyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate, except that 40 equivalents of TFA was used. After completion, the reaction was concentrated under reduced pressure, and the residue was purified by Prep HPLC (Method G) to afford ((3R,5R)-3,5-dimethylmorpholino)(5-fluoro-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)phenyl)methanone as a solid.

Yield: 20.52%; ¹H NMR (400 MHz, DMSO-d₆): δ 8.30-8.24 (m, 1H), 7.72-7.67 (m, 1H), 7.40-7.35 (m, 1H), 7.32-7.25 (m, 1H), 7.00 (dd, J=4.4, 9.0 Hz, 1H), 4.15 (br s, 2H), 4.02 (br s, 2H), 3.94-3.77 (m, 2H), 3.69 (br dd, J=2.1, 11.1 Hz, 2H), 3.50 (dt, J=2.3, 11.4 Hz, 2H), 3.20-3.12 (m, 1H), 3.06-2.95 (m, 1H), 2.90-2.80 (m, 1H), 2.65-2.54 (m, 3H), 2.47-2.38 (m, 2H), 2.31-2.10 (m, 4H), 2.10-1.97 (m, 3H), 1.85-1.76 (m, 3H), 1.65-1.58 (m, 1H), 1.36-1.26 (m, 1H), 1.25-1.12 (m, 6H); LCMS (Method D): Rt=1.47 min, m/z=581.2 [M+H]⁺; HPLC (Method D): Rt=2.01 min, 97.25%; Chiral SFC (Method R); Rt=3.98 min, 97.26%.

### Example 14. 5-Fluoro-N-isopropyl-N-((1r,3r)-3-methyl-3-(pyrrolidin-1-yl)cyclobutyl)-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide (Compound No. 14)

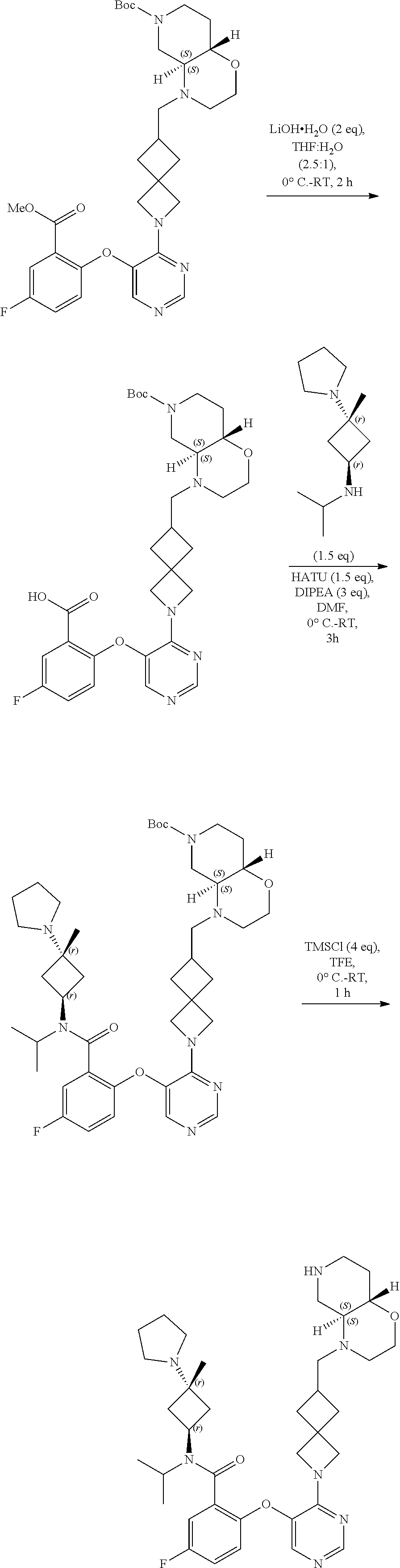

### Step 1. 2-((4-(6-(((4aS,8aS)-6-(tert-Butoxycarbonyl)octahydro-41-pyrido)[4,3-b][1,4]oxazin-4-yl)methyl)-2-azaspiro[3.3]heptan-2-yl)pyrimidin-5-yl)oxy)-5-fluorobenzoic acid

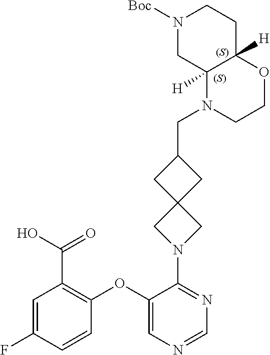

To a stirred solution of tert-butyl (4aS,8aS)-4-((2-(5-(4-fluoro-2-(methoxycarbonyl)phenoxy)pyrimidin-4-yl)-2-azaspiro[3.3]heptan-6-yl)methyl)hexahydro-2H-pyrido[4,3-b][1,4]oxazine-6(5H)-carboxylate (0.5 g, 0.837 mmol)) in 2.5:1 THF:H₂O (5 mL:2 mL), LiOH·H₂O (0.070 g, 1.673 mmol) was added at 0° C. The reaction was stirred at RT for 2 h, and monitored by LCMS and TLC (10% MeOH in DCM). After completion, the reaction was concentrated under reduced pressure. The residue was dissolved in water (10 mL), and the solution was acidified with 1.5 N HCl (5 mL) (pH ~6). The mixture was extracted with 10% MeOH in DCM (3×10 mL). The combined organic layer was dried over anhydrous sodium sulfate and filtered, and the filtrate was concentrated under reduced pressure to afford crude 2-((4-(6-(((4S,8aS)-6-(tert-butoxycarbonyl)octahydro-4H-pyrido[4,3-b][1,4]oxazin-4-yl)methyl)-2-azaspiro[3.3]heptan-2-yl)pyrimidin-5-yl)oxy)-5-fluorobenzoic acid (0.390 g, 73.5% yield) as a solid. This material was used without further purification. LCMS (Method G): Rt=1.62 min, m/z=584.3 [M+H]⁺, 91.98%.

### Step 2. tert-Butyl (4aS,8aS)-4-((2-(5-(4-fluoro-2-(isopropyl((1r,3r)-3-methyl-3-(pyrrolidin-1-yl)cyclobutyl)carbamoyl)phenoxy)pyrimidin-4-yl)-2-azaspiro[3.3]heptan-6-yl)methyl)hexahydro-2H-pyrido[4,3-b][1,4]oxazine-6(5H)-carboxylate

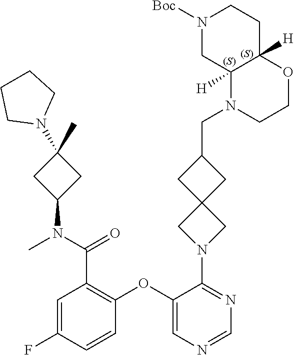

This compound was synthesized following the procedure described for the synthesis of Example 13, step 2, starting with 1 equivalent of 2-((4-(6-(((4aS,8aS)-6-(tert-butoxycarbonyl)octahydro-4H-pyrido[4,3-b][1,4]oxazin-4-yl)methyl)-2-azaspiro[3.3]heptan-2-yl)pyrimidin-5-yl)oxy)-5-fluorobenzoic acid and 1.5 equivalents of (1r,3r)-N-isopropyl-3-methyl-3-(pyrrolidin-1-yl)cyclobutan-1-amine. After completion, the reaction was concentrated under reduced pressure, and the crude was purified by reverse phase chromatography using a SiliCycle SiliaSep C18 column (80 g) on Biotage isolera one (Mobile phase A: 10 mM NH₄HCO: in water, B: ACN) to obtain tert-butyl (4aS,8aS)-4-((2-(5-(4-fluoro-2-(isopropyl((1r,3r)-3-methyl-3-(pyrrolidin-1-yl)cyclobutyl)carbamoyl)phenoxy)pyrimidin-4-yl)-2-azaspiro[3.3]heptan-6-yl)methyl)hexahydro-2H-pyrido[4,3-b][1,4]oxazine-6(5H)-carboxylate as a solid.

Yield: 30.7%; LCMS (Method E): Rt=2.34 min, m/z=762.3 [M+H]⁺, 51.23%.

### Step 3. S-Fluoro-N-isopropyl-N-((1r,3r)-3-methyl-3-(pyrrolidin-1-yl)cyclobutyl)-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide

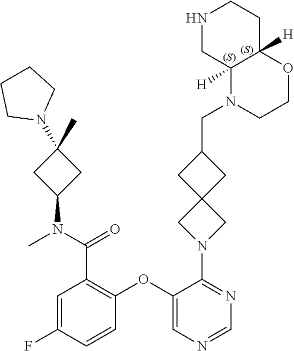

To a stirred solution of tert-butyl (4aS,8aS)-4-((2-(5-(4-fluoro-2-(isopropyl((1r,3r)-3-methyl-3-(pyrrolidin-1-yl)cyclobutyl)carbamoyl)phenoxy)pyrimidin-4-yl)-2-azaspiro[3.3]heptan-6-yl)methyl)hexahydro-2H-pyrido[4,3-b][1,4]oxazine-6(5H)-carboxylate (360 mg, 0.472 mmol) in TFE (6 mL), TMSCl (0.242 mL, 1.890 mmol) was added at 0° C. The reaction was stirred at RT for 1 b, and monitored by LCMS and TLC (10%/6 MeOH in DCM). After completion, the reaction was concentrated under reduced pressure. The crude was dissolved in DCM (6 mL) and TEA (0.263 mL, 1.890 mmol) was added at RT. The reaction was stirred at RT for 15 min. The reaction was concentrated under reduced pressure, and the crude was purified by reverse phase chromatography using a SiliCycle SiliaSep C18 column (80 g) on Biotage isolera one (Mobile phase A: 10 mM NH₄HCO in water, B: ACN). The fractions were concentrated under reduced pressure and lyophilized to afford 5-fluoro-N-isopropyl-N-((1r,3r)-3-methyl-3-(pyrrolidin-1-yl)cyclobutyl)-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide (134 mg, 41.5% yield) as a solid. ¹H NMR (400 MHz, DMSO-d₆): δ 8.29-8.22 (m, 1H), 7.79-7.55 (m, 1H), 7.31-7.01 (m, 3H), 4.29-3.93 (m, 5H), 3.89-3.64 ((m, 3H), 3.57-3.40 (m, 1H), 3.22-3.10 (m, 1H), 3.06-2.95 (m, 1H), 2.89-2.78 (m, 2H), 2.67-2.54 (m, 4H), 2.47-2.35 (m, 3H), 2.31-2.13 (m, 5H), 2.11-1.93 (m. 3H), 1.86-1.74 (m, 4H), 1.73-1.56 (m, 5H), 1.47-1.24 (m, 5H), 1.11-0.91 (m, 5H); LCMS (Method G): Rt=0.98 min, m/z=662.4 [M+H]⁺; HPLC (Method G): Rt=6.41 min, 96.82%; Chiral SFC (Method T): Rt=2.66 min, 98.07%.

### Example 15. 5-Fluoro-N-isopropyl-N-((1r,3r)-3-methyl-3-(pyrrolidin-1-yl)cyclobutyl)-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamide (Compound No. 15)

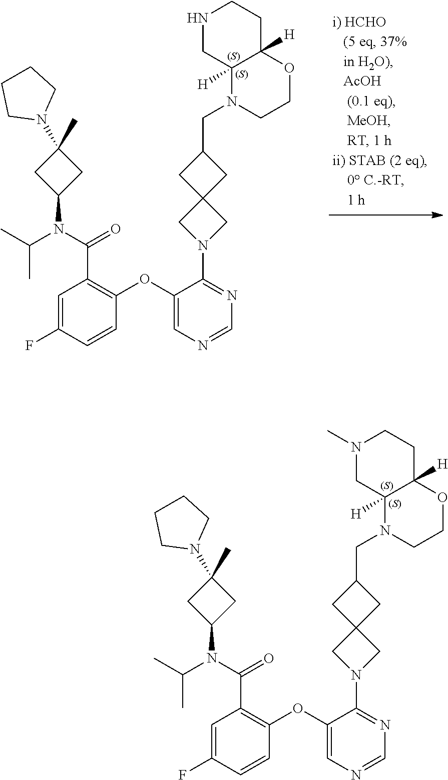

The reaction was performed following the same procedure in two batches (2×40 mg).

To a stirred solution of 5-fluoro-N-isopropyl-N-((1r,3r)-3-methyl-3-(pyrrolidin-1-yl)cyclobutyl)-2-((4-(6-(((4aS,8aS)-octahydro-4H-pyrido[4,3-b][1,4]oxazin-4-yl))methyl)-2-azaspiro[3.3]heptan-2-yl)pyrimidin-5-yl)oxy)benzamide (40 mg, 0.060 mmol) in MeOH (2 mL), acetic acid (0.363 mg, 6.04 μmol) and formaldehyde (0.030 mL, 0.302 mmol, 37% in H₂O) were added at RT. The reaction was stirred at RT for 1 h. To this reaction mixture, sodium triacetoxyborohydride (25.6 mg, 0.121 mmol) was added at 0° C., and the reaction was stirred at RT for 1 h. The reaction was monitored by LCMS. After completion, the reaction was concentrated under reduced pressure. The residue was dissolved in 10% MeOH in DCM (10 mL) and the solution was basified with saturated sodium bicarbonate solution (5 mL). The layers were separated, and the aqueous layer was extracted with 10% MeOH-DCM (2×10 mL). The combined organic layer was dried over anhydrous sodium sulfate and filtered, and the filtrate was concentrated under reduced pressure. The crude product from this batch and an impure product from other batch were combined and purified by reverse phase chromatography using a SiliCycle SiliaSep C18 column (40 g) on Biotage isolera one (Mobile phase A: 0.1% Formic acid in water, B: ACN). The fractions were concentrated under reduced pressure. The crude was basified with aqueous sodium bicarbonate (5 mL) and the mixture was extracted with 10% MeOH in DCM (2×5 mL). The combined organic layer was dried over anhydrous sodium sulfate and filtered, and the filtrate was concentrated under reduced pressure and lyophilized to afford 5-fluoro-N-isopropyl-N-((1r,3r)-3-methyl-3-(pyrrolidin-1-yl)cyclobutyl)-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamide (41 mg, 50.07% yield) as a solid. ¹H NMR (400 MHz, DMSO-d₆): δ 8.30-8.22 (m, 1H), 7.81-7.54 (m, 1H), 7.31-6.99 (m, 3H), 4.28-3.80 (m, 5H), 3.77-3.64 (m, 2H), 3.54-3.43 (m, 1H), 3.10-2.98 (m, 1H), 2.97-2.81 (m, 2H), 2.76-2.69 (m, 1H), 2.65-2.54 (m, 3H), 2.43-2.34 (m, 2H), 2.31-2.20 (m, 3H), 2.17 (s, 31H), 2.16-2.03 (m, 3H), 2.01-1.87 (m, 3H), 1.85-1.76 (m, 31H), 1.75-1.53 (m, 6H), 1.52-1.40 (m, 3H), 1.36-1.22 (m, 3H), 1.13-0.90 (m, 5H); LCMS (Method G): Rt=1.02 min, m/z=676.4 [M+H]⁺; HPLC (Method G): Rt=6.46 min, 99.66%.

### Example 16. N-(2,2-Difluoroethyl)-5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide (Compound No. 16)

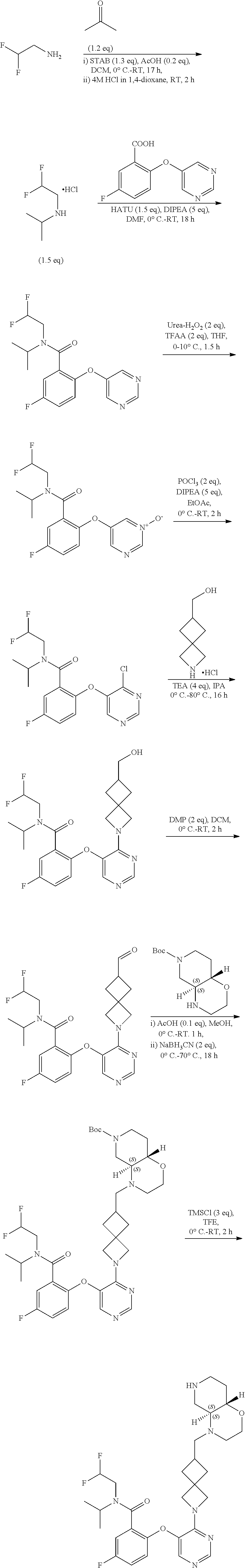

### Step 1. N-(2,2-Difluoroethyl)propan-2-amine hydrochloride

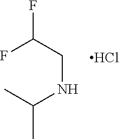

In a 2 L four neck round bottom flask under a nitrogen atmosphere, 2,2-difluoroethan-1-amine (21.74 mL, 308 mmol) was dissolved in DCE (500 mL), and the solution was cooled to 0° C. To this solution, acetone (27.6 mL, 370 mmol) and acetic acid (3.53 mL, 61.7 mmol) were added, and the reaction was stirred at RT for 1 h. The reaction was again cooled to 0° C., and sodium triacetoxyborohydride (85 g, 401 mmol) was added portion-wise over 15 min. The reaction was stirred at RT for 16 h, and monitored by TLC (60% EtOAc in hexane). After completion, the reaction was diluted with DCM (500 mL) and washed with 10% NaHCO₃ solution (3×500 mL). The combined organic layer was dried over Na₂SO₄ and filtered. The filtrate was transferred to 2 L single neck round bottom flask and cooled to 0° C. To this solution, 4 M HCl in 1,4 dioxane (300 mL) was added, and the mixture was stirred for 2 h at RT. The reaction was concentrated under reduced pressure to obtain crude N-(2,2-difluoroethyl)propan-2-amine hydrochloride (33 g, 67% yield) as a solid. This material was used without further purification. ¹H NMR (400 MHz, DMSO-d₄): δ 9.79-9.65 (br s, 2H), 6.69-6.38 (m, 1H), 3.53-3.35 (m, 3H), 1.28 (d, J=6.5 Hz, 6H).

### Step 2. N-(2,2-Difluoroethyl)-5-fluoro-N-isopropyl-2-(pyrimidin-5-yloxy)benzamide

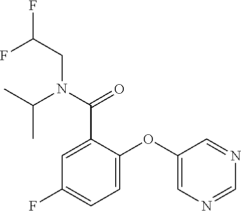 [SMILES: CC(C)N(CC(F)F)C(=O)c1cc(F)ccc1Oc1cncnc1]

In a 250 mL two neck round bottom flask under a nitrogen atmosphere, 5-fluoro-2-(pyrimidin-5-yloxy)benzoic acid (2.9 g, 12.38 mmol) was dissolved in DMF (30 mL). To this solution, N-(2,2-difluoroethyl)propan-2-amine hydrochloride (2.96 g, 18.57 mmol), DIPEA (10.79 mL, 61.9 mmol) and HATU (7.06 g, 18.57 mmol) were added at 0° C. under nitrogen atmosphere. The reaction was stirred at RT for 18 h, and monitored by TLC (50%1 EtOAc in hexane). After completion, the reaction was quenched with ice-cold water (100 mL) and extracted with EtOAc (3×100 mL). The combined organic layer was dried over anhydrous sodium sulfate and filtered, and the filtrate was concentrated under reduced pressure to obtain the crude compound. The crude was purified by silica gel flash column chromatography using EtOAc in hexane (the product eluted at 25-30% EtOAc in hexane) to obtain N-(2,2-difluoroethyl)-5-fluoro-N-isopropyl-2-(pyrimidin-5-yloxy)benzamide (4.2 g, 97% yield) as a liquid. ¹H NMR (400 MHz, DMSO-d₆): δ 8.97 (s, 1H), 8.55 (s, 2H), 7.46-7.27 (m, 3H), 6.07 (tt, J=4.4, 56.9 Hz, 1H), 3.88-3.81 (m, 1H), 3.76-3.45 (m, 2H), 1.09 (d, J=6.5 Hz, 3H), 1.02 (d, J=6.6 Hz, 3H); LCMS: (Method D): Rt=1.69 min, m/z=340.0 [M+H]⁺, 96.91%.

### Step 3. 5-(2-((2,2-Difluoroethyl)(isopropyl)carbamoyl)-4-fluorophenoxy)pyrimidine 1-oxide

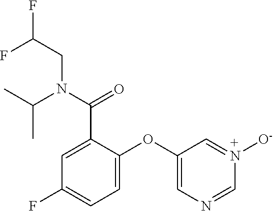

In a 100 mL two neck round bottom flask under a nitrogen atmosphere, N-(2,2-difluoroethyl)-5-fluoro-N-isopropyl-2-(pyrimidin-5-yloxy)benzamide (2.2 g, 6.48 mmol) was dissolved in THE (25 mL) at 0° C. To this solution, urea hydrogen peroxide (1.220 g, 12.97 mmol) and TFAA (1.832 mL, 12.97 mmol) were added slowly. The reaction was stirred at 0° C.-10° C. for 1.5 h, and monitored by TLC (70% EtOAc in hexane). After completion, the reaction was quenched with saturated sodium bicarbonate solution (30 mL). The temperature was maintained at 0-10° C. and the mixture was extracted with DCM (3×100 mL). The combined organic layer was washed with 1 M Na₂S₂O₃ solution (50 mL), dried over anhydrous sodium sulfate and filtered, and the filtrate was concentrated under reduced pressure to obtain crude 5-(2-((2,2-difluoroethyl)(isopropyl)carbamoyl)-4-fluorophenoxy)pyrimidine 1-oxide (2.5 g, 99% yield) as a semi-solid. This material was used without further purification. LCMS (Method D): Rt=1.48 min, m/z=356.2 [M+H]⁺, 91.14%.

### Step 4. 2-((4-Chloropyrimidin-5-yl)oxy)-N-(2,2-difluoroethyl)-S-fluoro-N-isopropylbenzamide

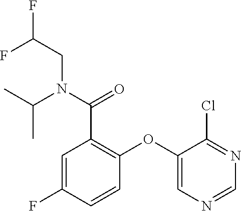 [SMILES: CC(C)N(CC(F)F)C(=O)c1cc(F)ccc1Oc1cncnc1Cl]

In a 100 mL two neck round bottom flask under nitrogen atmosphere, 5-(2-((2,2-difluoroethyl)(isopropyl)carbamoyl)-4-fluorophenoxy)pyrimidine 1-oxide (2.5 g, 7.04 mmol) was dissolved in EtOAc (25 mL). To this solution, DIPEA (6.14 mL, 35.2 mmol) and POCl₃ (1.312 mL, 14.07 mmol) were added at 0° C., and the reaction was stirred at RT for 2 h. The progress of the reaction was monitored by TLC (50% EtOAc in hexane). After completion, the reaction was concentrated under reduced pressure to obtain the crude compound. The crude was purified by silica gel flash column chromatography by using EtOAc in hexane (product eluted at 25-30% EtOAc in hexane) to obtain 2-((4-chloropyrimidin-5-yl)oxy)-N-(2,2-difluoroethyl)-5-fluoro-N-isopropylbenzamide (1.3 g, 47.9% yield) as a semi-solid. ¹H NMR (400 MHz, DMSO-d₆); δ 8.83 (s, 1H), 8.41 (s, 1H), 7.46 (dd, J=3.2, 7.6 Hz, 1H), 7.38 (spt, J=2.6 Hz, 2H), 6.24-5.94 (m, 1H), 3.88-3.70 (m, 1H), 3.68 (s, 2H), 1.09 (d, J=6.5 Hz, 6H); LCMS (Method D): Rt=1.87 min, m/z=374.0 [M+H]⁺, 96.89%.

### Step 5. N-(2,2-Difluoroethyl)-5-fluoro-2-((4-(6-(hydroxymethyl)-2-azaspiro[3.3]heptan-2-yl)pyrimidin-5-yl)oxy)-N-isopropylbenzamide

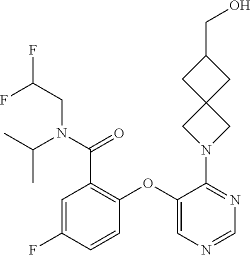 [SMILES: CC(C)N(CC(F)F)C(=O)c1cc(F)ccc1Oc1cncnc1N1CC2(CC(CO)C2)C1]

This compound was synthesized following the procedure described for the synthesis of Intermediate 11, starting with 1 equivalent of 2-((4-chloropyrimidin-5-yl)oxy)-N-(2,2-difluoroethyl)-5-fluoro-V-isopropylbenzamide and 1 equivalent of 2-azaspiro[3.3]heptan-6-yl)methanol hydrochloride, except that 4 equivalents of TEA was used, and the reaction was stirred at 80° C. After work-up, the crude compound was purified by silica gel flash column chromatography using EtOAc in hexane (product eluted at 70% EtOAc in hexane) to obtain N-(2,2-difluoroethyl)-5-fluoro-2-((4-(6-(hydroxymethyl)-2-azaspiro[3.3]heptan-2-yl)pyrimidin-5-yl)oxy)-N-isopropylbenzamide as a semi-solid.

Yield: 37.8%; ¹H NMR (400 MHz, DMSO-d₆): δ 8.35-8.23 (m, 1H), 7.84-7.70 (m, 1H), 7.35 (dd, J=3.1, 8.3 Hz, 1H), 7.31-7.24 (m, 1H), 6.97 (dd, J=4.4, 9.1 Hz, 1H), 6.41-6.02 (m, 1H), 4.49 (br t, J=5.1 Hz, 1H), 4.18-4.05 (m, 2H), 4.00-3.93 (m, 1H), 3.89-3.65 (m, 3H), 3.32-3.28 (m, 2H), 2.24-2.09 (m, 3H), 1.94-1.84 ((m, 2H), 1.27-1.23 (m, 1H), 1.09 (br dd, J=6.5, 13.9 Hz, 6H); LCMS (Method D): Rt=1.63 min, m/z=465.1 [M+H]⁺, 83.97%.

### Step 6. N-(2,2-Difluoroethyl)-5-fluoro-2-((4-(6-formyl-2-azaspiro[3.3]heptan-2-yl)pyrimidin-5-yl)oxy)-N-isopropylbenzamide

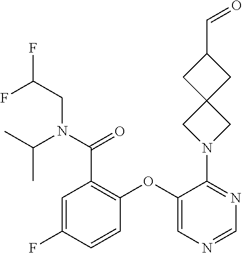 [SMILES: CC(C)N(CC(F)F)C(=O)c1cc(F)ccc1Oc1cncnc1N1CC2(CC(C=O)C2)C1]

This compound was synthesized following the procedure described for the synthesis of Intermediate 12, starting with 1 equivalent of N-(2,2-difluoroethyl)-5-fluoro-2-((4-(6-(hydroxymethyl)-2-azaspiro[3.3]heptan-2-yl)pyrimidin-5-yl)oxy)-N-isopropylbenzamide, except that 2 equivalents of DMP was used. After completion, the reaction was filtered through Celite®, and the pad was washed with DCM. The filtrate was washed with saturated sodium bicarbonate solution. The organic layer was dried over anhydrous sodium sulfate and filtered, and the filtrate was concentrated under reduced pressure to afford crude N-(2,2-difluoroethyl)-5-fluoro-2-((4-(6-formyl-2-azaspiro[3.3]heptan-2-yl)pyrimidin-5-yl)oxy)-N-isopropylbenzamide as a solid. This material was used without further purification.

Yield: 27.4%; LCMS (Method D): Rt=1.78 min, m/z=463.1 [M+H]⁺, 28.33%.

### Step 7. tert-Butyl (4aS,8aS)-4-((2-(S-(2-((2,2-difluoroethyl)(isopropyl)carbamoyl)-4-fluorophenoxy)pyrimidin-4-yl)-2-azaspiro[3.3]heptan-6-yl)methy)hexahydro-2H-pyrido[4,3-b][1,4]oxazine-6(5H)-carboxylate

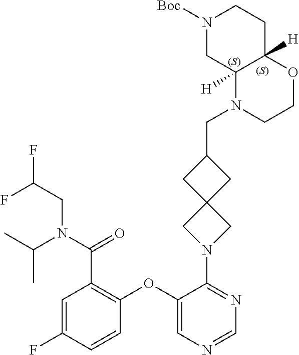

In a 25 mL two neck round bottom flask under a nitrogen atmosphere, N-(2,2-difluoroethyl)-5-fluoro-2-((4-(6-formyl-2-azaspiro[3.3]heptan-2-yl)pyrimidin-5-yl)oxy)-N-isopropylbenzamide (200 mg, 0.432 mmol) and tert-butyl (4aS,8aS)-hexahydro-2H-pyrido[4,3-b][1,4]oxazine-6(5H)-carboxylate (105 mg, 0.432 mmol) were dissolved in MeOH (5 mL), and AcOH (2.476 μL, 0.043 mmol) was added at 0° C. The reaction was stirred at 0° C. for 1 h. To this reaction mixture, NaBH₃CN (34.5 mg, 0.865 mmol) was added at 0° C., and the reaction was stirred at 70° C. for 18 h. The reaction was monitored by TLC (10% MeOH in DCM). After completion, the reaction was quenched with water (15 mL) and the mixture was extracted with EtOAc (2×50 mL). The combined organic layer was dried over anhydrous sodium sulfate and filtered, and the filtrate was concentrated under reduced pressure to obtain crude tert-butyl (4aS,8aS)-4-((2-(5-(2-((2,2-difluoroethyl)(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (250 mg, 51.7% yield) as a solid. This material was used without further purification. LCMS (Method D): Rt=2.08 min, m/z=689.2 [M+H]⁺, 61.63%.

### Step 8. N-(2,2-Difluoroethyl)-S-fluoro-N-isopropyl-2-((4-(6-(((4a,8aS)-octahydro-4H-pyrido[4,3-b][1,4]oxazin-4-yl)methyl)-2-azaspiro[3.3]heptan-2-yl)pyrimidin-5-yl)oxy)benzamide

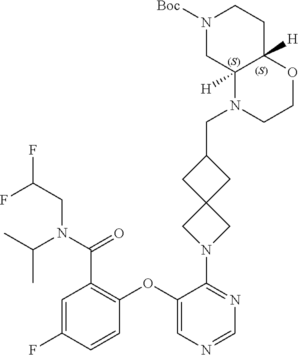

This compound was synthesized following the procedure described for the synthesis of Example 1, step 2, starting with 1 equivalent of tert-butyl (4aS,8aS)-4-((2-(5-(2-((2,2-difluoroethyl)(isopropyl)carbamoyl)-4-fluorophenoxy)pyrimidin-4-yl)-2-azaspiro[3.3]heptan-6-yl)methyl)hexahydro-2H-pyrido[4,3-b][1.4]oxazine-6(5H)-carboxylate, except that 3 equivalents of TMSCl was used. After completion, the reaction was concentrated under reduced pressure. The crude was purified by Prep-HPLC (Method C) to afford N-(2,2-difluoroethyl)-5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide as a solid.

Yield: 26.5%; ¹H NMR (400 MHz, DMSO-d₆); δ 8.33-8.26 (m, 1H), 7.83-7.69 (m, 1H), 7.39-7.22 (m, 2H), 7.01-6.91 (m, 1H), 6.39-6.04 (m, 1H), 4.21-3.99 (m, 3H), 3.98-3.80 (m, 2H), 3.79-3.64 (m, 3H). 3.54-3.46 (m, 1H), 3.22-3.11 (m, 1H), 3.05-2.97 (m, 1H), 2.91-2.76 (m, 1H), 2.65-2.56 (m, 1H), 2.44-2.38 (m, 1H), 2.31-2.11 (m, 5H), 2.10-1.96 (m, 2H), 1.89-1.72 (m, 3H), 1.69-1.51 (m, 1H), 1.37-1.22 (m, 2H), 1.14-1.02 (m, 6H); LCMS (Method D): Rt=1.62 min, m/z=589.1 [M+H]⁺; HPLC (Method A): Rt=4.75 min, 99.37%; Chiral SFC (Method U): Rt=3.93 min. 98.87%.

### Example 17. N-Ethyl-5-fluoro-N-isopropyl-2-((4-(6-(((4aR,8aR)-3-oxooctahydro-4H-pyrido[4,3-b][1,4]oxazin-4-yl)methyl)-2-azaspiro[3.3]heptan-2-yl)pyrimidin-5-yl)oxy)benzamide (Compound No. 17)

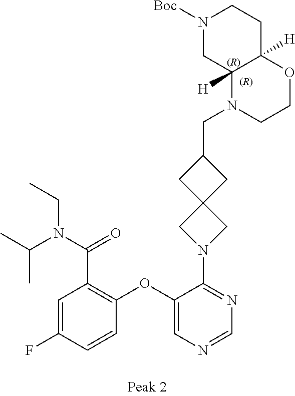

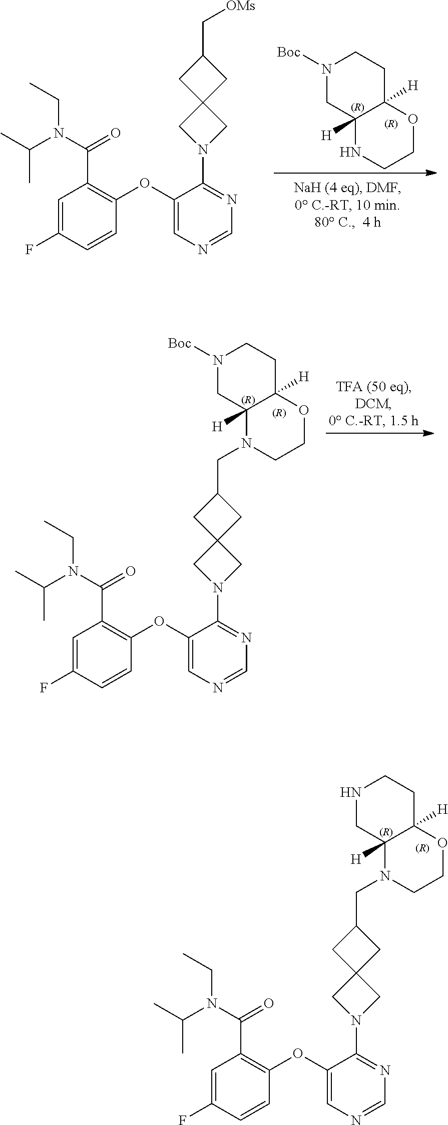

### Step 1. tert-Butyl (4aR,8aR)-4-(2-(3-(2-(ethyl(isopropyl)carbamoyl)-4-fluorophenoxy)pyrimidin-4-yl)-2-azaspiro[3.3]heptan-6-yl)methyl)-3-oxohexahydro-2H-pyrido[4,3-b][1.4]oxazine-6(5H)-carboxylate and tert-butyl (4aR,8aR)-3-((2-(5-(2-(ethyl(isopropyl)carbamoyl)-4-fluorophenoxy)pyrimidin-4-yl)-2-azaspiro[3.3]heptan-6-yl)methoxy)-4a,7,8,8a-tetrahydro-2H-pyrido[4,3-b][1,4]oxazine-6(5H)-carboxylate

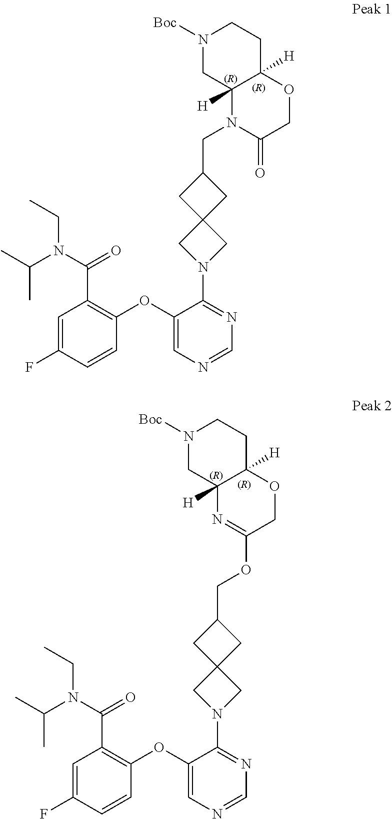

To a stirred solution of tert-butyl (4aR,8aR)-3-oxohexahydro-2H-pyrido[4,3-b][1,4]oxazine-6(5H)-carboxylate (0.225 g, 0.878 mmol) in dry DMF (6 mL), NaH (0.140 g, 3.51 mmol) was added at 0° C. The reaction was stirred at the same temperature for 5 min, then (2-(5-(2-(ethyl(isopropyl)carbamoyl)-4-fluorophenoxy)pyrimidin-4-yl)-2-azaspiro[3.3]heptan-6-yl)methyl methanesulfonate (0,378 g, 0,746 mmol) was added. The reaction was stirred at RT for min, and then at 80° C. for 4 h under a nitrogen atmosphere. The reaction was monitored by TLC (10% MeOH in DCM). After completion, the reaction was quenched with saturated sodium chloride (50 mL), and the mixture was extracted with ethyl acetate (2×50 mL). The combined organic layer was washed with brine (50 mL), dried over anhydrous sodium sulfate and filtered, and the filtrate was concentrated under reduced pressure. The crude material was purified by reverse phase chromatography using a SiliCycle SiliaSep C18 column on Biotage isolera one (Mobile phase A: 10 mM NH₄HCO₃ in water, B: ACN) to afford both regioisomers.

Peak 1: tert-Butyl (4aR,8aR)-4-((2-(5-(2-(ethyl(isopropyl)carbamoyl)-4-fluorophenoxy)pyrimidin-4-yl)-2-azaspiro[3.3]heptan-6-yl)methyl)-3-oxohexahydro-2H-pyrido[4,3-b][1,4]oxazine-6(5H)-carboxylate (0.173 g, 29% yield) as a solid. ¹H NMR (400 MHz, DMSO-d₆): δ 8.30-8.24 (m, 1H), 7.77-7.64 (m, 1H), 7.33-7.19 (m, 2H), 7.08-6.97 (m, 1H), 4.40-3.92 (m, 8H), 3.82-3.70 (m, 1H), 3.62-3.49 (m, 1H), 3.45-3.34 (m, 1H), 3.29-3.20 (m, 1H), 3.04-2.91 (m, 2H), 2.88-2.70 (m, 1H), 2.43-2.35 (m, 1H), 2.29-2.18 (m, 2H), 2.04-1.92 (m, 1H), 1.90-1.78 (m, 2H), 1.41 (s, 9H), 1.22-0.97 (m, 9H), two three merged with solvent peaks; LCMS (Method D): Rt=1.97 min, m/z=667.3 [M+H]⁺, 98.16%.

Peak 2: tert-Butyl (4aR,8aR)-3-((2-(5-(2-(ethyl(isopropyl)carbamoyl)-4-fluorophenoxy)pyrimidin-4-yl)-2-azaspiro[3.3]heptan-6-yl)methoxy)-4a,7,8,8a-tetrahydro-2H-pyrido[4,3-b][1,4]oxazine-6(5H)-carboxylate (0.029 g, 3.91% yield) as a solid. ¹H NMR (400 MHz, DMSO-d₆): δ 8.33-8.21 (m, 1H), 7.78-7.61 (m, 1H), 7.36-7.17 (m, 2H), 7.10-6.94 (m, 1H), 4.46-3.86 ((m, 9H), 3.80-3.70 ((m, 1H), 3.46-3.36 (m, 1H), 3.28-3.04 (m, 2H), 3.01-2.72 (m, 2H), 2.46-2.39 (m, 1H), 2.30-2.11 (m, 2H), 2.01-190 (m, 2H), 1.88-1.80 (m, 1H), 1.44-1.36 (m, 9H), 1.22-0.98 (m, 9H), three protons merged with solvent peaks; LCMS (Method D): Rt=2.20 min, m/z=667.2 [M+H]⁺, 79.31%.

### Step 2. N-Ethyl-5-fluoro-N-isopropyl-2-((4-(6-(((4aR,8aR)-3-oxooctahydro-4H-pyrido[4,3-b][1,4]oxazin-4-yl)methyl)-2-azaspiro[3.3]heptan-2-yl)pyrimidin-5-yl)oxy)benzamide

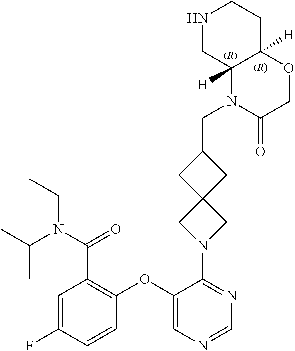

This compound was synthesized following the procedure described for the synthesis of Example 2, step 1, starting with 1 equivalent of tert-butyl (4aR,8aR)-4-((2-(5-(2-(ethyl(isopropyl)carbamoyl)-4-fluorophenoxy)pyrimidin-4-yl)-2-azaspiro[3.3]heptan-6-yl)methyl)-3-oxohexahydro-2H-pyrido[4,3-b][1,4]oxazine-6(5H)-carboxylate, except that 50 equivalents of TFA was used. After completion, the reaction was concentrated under reduced pressure. The crude material was purified by Prep-HPLC (Method F) to afford N-ethyl-5-fluoro-N-isopropyl-2-((4-(6-(((4aR,8aR)-3-oxooctahydro-4H-pyrido[4,3-b][1,4]oxazin-4-yl)methyl)-2-azaspiro[3.3]heptan-2-yl)pyrimidin-5-yl)oxy)benzamide as a solid.

Yield: 53.9%; ¹H NMR (400 MHz, DMSO-d₆): δ 8.29-8.23 (m, 1H), 7.77-7.65 (m, 1H), 7.34-7.21 (m, 21H), 7.08-6.97 (m, 1H), 4.49-3.96 (m, 6H), 3.82-3.67 (m, 2H), 3.45-3.38 (m, 1H), 3.31-3.09 (m, 2H), 3.05-2.89 (m, 3H), 2.48-2.35 (m, 2H), 2.28-2.10 (m, 3H), 1.98-1.90 (m, 1H), 1.88-1.75 (m, 2H), 1.43-1.30 (m, 1H), 1.23-0.96 (m, 9H), two protons merged with solvent peaks; LCMS (Method D): Rt=1.52 min, m/z=567.3 [M+H]⁺; HPLC (Method A): Rt=4.35 min, 99.35%; Chiral SFC (Method K): Rt=3.28 min, 99.70%.

### Example 18. N-Ethyl-5-fluoro-N-isopropyl-2-((4-(6-(((4aS,8aS)-3-oxooctahydro-4H-pyrido[4,3-b][1,4]oxazin-4-yl)methyl)-2-azaspiro[3.3]heptan-2-yl)pyrimidin-5-yl)oxy)benzamide (Compound No. 18)

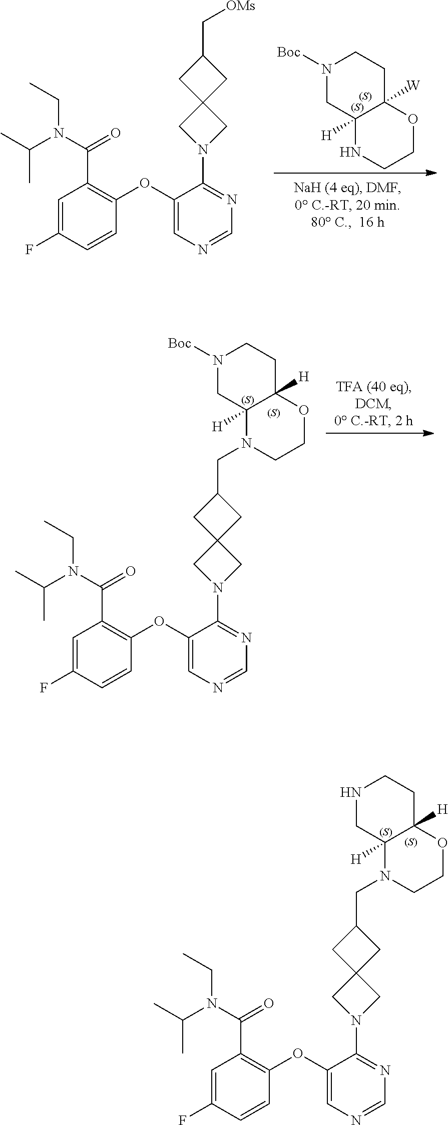

### Step 1. tert-Butyl (4aS,8aS)-4-((2-(5-(2-(ethyl(isopropyl)carbamoyl)-4-fluorophenyl)pyrimidin-4-yl)-2-azaspiro[3.3]heptan-6-yl)methyl)-3-oxohexahydro-2H-pyrido[4,3-b][1,4]oxazine-6(5H)-carboxylate

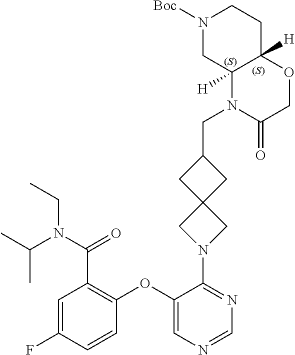

This compound was synthesized following the procedure described for the synthesis of Example 17, step 1, starting with 1 equivalent of (2-(5-(2-(ethyl(isopropyl)carbamoyl)-4-fluorophenoxy)pyrimidin-4-yl)-2-azaspiro[3.3]heptan-6-yl)methyl methanesulfonate and 1.48 equivalents of tert-butyl (4aS,8aS)-3-oxohexahydro-2H-pyrido[4,3-b][1,4]oxazine-6(5H)-carboxylate. After work-up, the crude material was purified by reverse phase chromatography using a SiliCycle SiliaSep C18 column on Biotage isolera one (Mobile phase A: 10 mM NH₄HCO₃ in water, B: ACN) to afford tert-butyl (4aS,8aS)-4-((2-(5-(2-(ethyl(isopropyl)carbamoyl)-4-fluorophenoxy)pyrimidin-4-yl)-2-azaspiro[3.3]heptan-6-yl))methyl)-3-oxohexahydro-2H-pyrido[4,3-b][1,4]oxazine-6(5H)-carboxylate as a solid.

Yield: 20.75%; ¹H NMR (400 MHz, DMSO-d₆): δ 8.33-8.22 (m, 1H), 7.78-7.61 (m, 1H), 7.35-7.20 (m, 2H), 7.07-6.97 (m, 1H), 4.46-3.85 (m, 8H), 3.83-3.69 (m, 1H), 3.61-3.46 (m, 1H), 3.45-3.37 (m, 1H), 3.28-3.09 (m, 3H), 3.05-2.72 (m, 3H), 2.42-2.36 (m, 1H), 2.30-2.14 (m, 3H), 2.04-1.94 (m, 1H), 1.91-1.78 (m, 2H), 1.41 (s, 9H), 1.22-0.95 (m, 9H); LCMS (Method D): Rt=1.94 min, m/z=667.2 [M+H]⁺, 95.28%.

Note: The other regioisomer was not isolated.

### Step 2. N-Ethyl-5-fluoro-N-isopropyl-2-((4-(6-(((4aS,8aS)-3-oxooctahydro-4H-pyrido[4,3-b][1,4]oxazin-4-yl)methy)-2-azaspiro[3.3]hepta-2-yl)pyrimidin-5-yl)oxy)benzamide

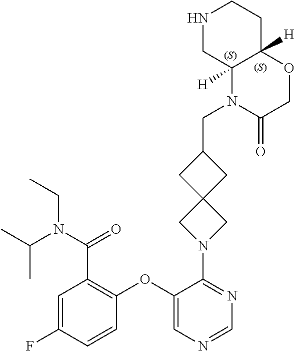

This compound was synthesized following the procedure described for the synthesis of Example 2, step 1, starting with 1 equivalent of tert-butyl (4aS,8aS)-4-((2-(5-(2-(ethyl(isopropyl)carbamoyl)-4-fluorophenoxy)pyrimidin-4-yl)-2-azaspiro[3.3]heptan-6-yl)methyl)-3-oxohexahydro-2H-pyrido[4,3-b][1,4]oxazine-6(5H)-carboxylate, except that 40 equivalents of TFA was used. After completion, the reaction was concentrated under reduced pressure. The crude was purified by Prep HPLC (Method L) to obtain N-ethyl-5-fluoro-N-isopropyl-2-((4-(6-(((4aS,8aS)-3-oxooctahydro-4H-pyrido[4,3-b][1,4]oxazin-4-yl)methyl)-2-azaspiro[3.3]heptan-2-yl)pyrimidin-5-yl)oxy)benzamide as a solid.

Yield: 45.5%; ¹H NMR (400 MHz, DMSO-d₆): δ 8.31-8.24 (m, 1H), 7.76-7.64 (m, 1H), 7.34-7.19 (m, 2H), 7.07-6.96 (m, 1H), 4.49-3.97 (m, 6H), 3.80-3.69 (m, 2H), 3.45-3.38 (m, 1H), 3.27-3.10 (m, 2H), 3.07-2.89 (m, 3H), 2.47-2.30 (m, 2H), 2.28-2.12 (m, 3H), 2.00-1.76 (m, 3H), 1.43-1.29 (m, 1H), 1.25-0.96 (m, 9H), two protons merged with solvent peaks; LCMS (Method D): Rt=1.54 min, m/z=567.3 [M+H]⁺; HPLC (Method A): Rt=4.35 min, 99.96%; Chiral SFC (Method K): Rt=2.90 min, 100%.

### Example 19. N-Ethyl-5-fluoro-N-isopropyl-2-((4-(6-((octahydro-1H-pyrrolo[2,3-c]pyridin-1-yl)methyl)-2-azaspiro[3.3]heptan-2-yl)pyrimidin-5-yl)oxy)benzamide (Compound No. 19)

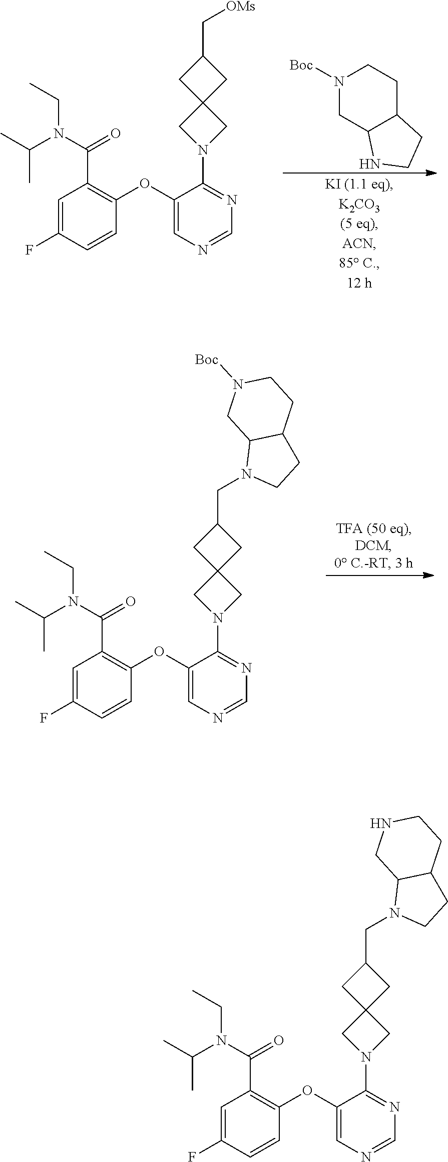 [SMILES: CCN(C(=O)c1cc(F)ccc1Oc1cncnc1N1CC2(CC(COS(C)(=O)=O)C2)C1)C(C)C ; CC(C)(C)OC(=O)N1CCC2CCNC2C1 ; CCN(C(=O)c1cc(F)ccc1Oc1cncnc1N1CC2(CC(CN3CCC4CCN(C(=O)OC(C)(C)C)CC43)C2)C1)C(C)C ; CCN(C(=O)c1cc(F)ccc1Oc1cncnc1N1CC2(CC(CN3CCC4CCNCC43)C2)C1)C(C)C]

### Step 1. tert-Butyl 1-((2-(5-(2-(ethyl(isopropyl)carbamoyl)-4-fluorophenoxy)pyrimidin-4-yl)-2-azaspiro[3.3]heptan-6-yl)methyl)octahydro-6H-pyrrolo[2,3-c]pyridine-6-carboxylate

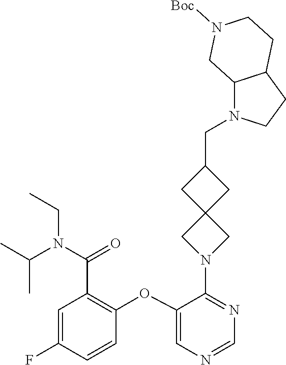 [SMILES: CCN(C(=O)c1cc(F)ccc1Oc1cncnc1N1CC2(CC(CN3CCC4CCN(C(=O)OC(C)(C)C)CC43)C2)C1)C(C)C]

To a stirred solution of (2-(5-(2-(ethyl(isopropyl)carbamoyl)-4-fluorophenoxy)pyrimidin-4-yl)-2-azaspiro[3.3]heptan-6-yl)methyl methanesulfonate (224 mg, 0.442 mmol) and tert-butyl octahydro-6H-pyrrolo[2,3-c]pyridine-6-carboxylate (100 mg, 0.442 mmol) in ACN (6 mL), potassium carbonate (305 mg, 2.209 mmol) and potassium iodide (81 mg, 0.486 mmol) were added. The reaction was heated to 85° C. for 12 h, and monitored by LCMS. After completion, the reaction was quenched with water (25 mL) and extracted with EtOAc (3×25 mL). The combined organic layer was dried over anhydrous sodium sulfate and filtered, and the filtrate was concentrated under reduced pressure to obtain tert-butyl 1-((2-(5-(2-(ethyl(isopropyl)carbamoyl)-4-fluorophenoxy)pyrimidin-4-yl)-2-azaspiro[3.3]heptan-6-yl)methyl)octahydro-6H-pyrrolo[2,3-c]pyridine-6-carboxylate (368 mg, quantitative yield) as a solid. This material was used without further purification. LCMS (Method D): Rt=2.31 min, m/z=637.2 [M+H]⁺, 54.10%.

### Step 2. N-Ethyl-S-fluoro-N-isopropyl-2-((4-(6-((octahydro-1H-pyrrolo[2,3-c]pyridin-1-yl)methyl)-2-azaspiro[3.3]heptan-2-yl)pyrimidin-5-yl)oxy)benzamide

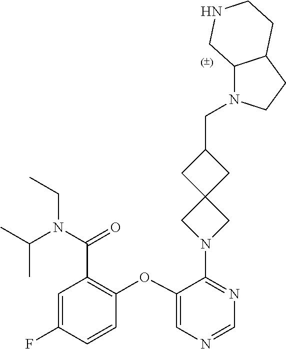 [SMILES: CCN(C(=O)c1cc(F)ccc1Oc1cncnc1N1CC2(CC(CN3CCC4CCNCC43)C2)C1)C(C)C]

This compound was synthesized following the procedure described for the synthesis of Example 2, step 1, starting with 1 equivalent of tert-butyl 1-((2-(5-(2-(ethyl(isopropyl)carbamoyl)-4-fluorophenoxy)pyrimidin-4-yl)-2-azaspiro[3.3]heptan-6-yl)methyl)octahydro-6H-pyrrolo[2,3-c]pyridine-6-carboxylate, except that 50 equivalents of TFA was used. After completion, the reaction was concentrated under reduced pressure. The crude material was purified by Prep HPLC (Method M) to N-thyl-5-fluoro-N-isopropyl-2-((4-(6-((octahydro-1H-pyrrolo[2,3-c]pyridin-1-yl)methyl)-2-azaspiro[3.3]heptan-2-yl)pyrimidin-5-yl)oxy)benzamide as a solid.

Yield: 7.73%; ¹H NMR (400 MHz, DMSO-d₆): δ 8.30-8.24 ((m, 1H), 7.76-7.65 (m, 1H), 7.33-7.22 (m, 2H), 7.01-6.95 (m, 1H), 4.45-3.96 (m, 4H), 3.81-3.70 (m, 1H), 3.47-3.39 (m, 1H), 3.29-3.10 (m, 2H), 3.05-2.94 (m, 1H), 2.85-2.71 (m, 2H), 2.65-2.55 (m, 1H), 2.47-2.35 (m, 1H), 2.29-2.10 (m, 5H), 2.08-1.99 (m, 3H), 1.90-1.70 (m, 3H), 1.43-1.28 (m, 3H), 1.24-0.96 (m, 9H), LCMS (Method D): Rt=1.71 min, m/z=537.2 [M+H]⁺; HPLC (Method A): Rt=5.12 min, 95.86%.

### Example 20. 2-((4-(6-((2-Amino-5,6-dihydropyrimidin-1(4H)-yl)methyl)-2-azaspiro[3.3]heptan-2-yl)pyrimidin-5-yl)oxy)-N-ethyl-5-fluoro-N-isopropylbenzamide (Compound No. 20)

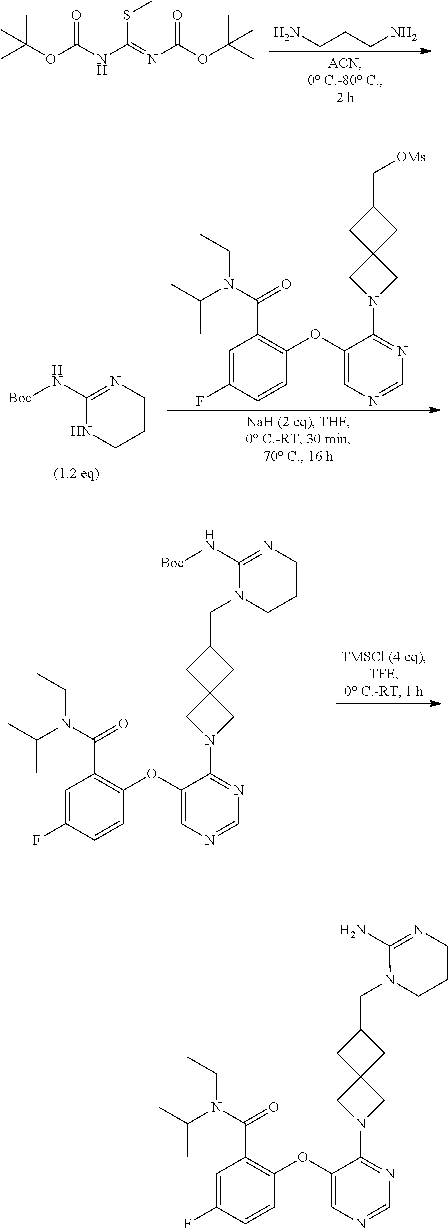

### Step 1. tert-butyl (1,4,5,6-tetrahydropyrimidin-2-yl)carbamate

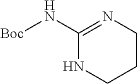 [SMILES: CC(C)(C)OC(=O)NC1=NCCCN1]

To a stirred solution of 1,3 diboc-2-methylisothiourea (2 g, 6.89 mmol) in ACN (20 mL), propane-1,3-diamine (0.580 mL, 6.89 mmol) was added at 0° C. under nitrogen atmosphere. The reaction was stirred at 80° C. for 2 h, then was concentrated under reduced pressure. The crude product was purified by Prep HPLC (method G) to obtain tert-butyl (1,4,5,6-tetrabydropyrimidin-2-yl)carbamate (200 mg, 14.27% yield) as a solid. ¹H NMR (400 MHz, DMSO-d₆): δ 8.03 (br s, 2H), 3.20 (t, J=5.8 Hz, 4H), 1.75 (quin, J=5.8 Hz, 2H), 1.34 (s, 9H); LCMS (Method D): Rt=1.22 min, m/z=200.1 [M+H]⁺, 97.94%.

### Step 2. tert-Butyl (1-((2-(5-(2-(ethyl(isopropyl)carbamoyl)-4-fluorophenoxy)pyrimidin-4-yl)-2-azaspiro[3.3]heptan-6-yl)methyl)-1,4,5,6-tetrahydropyrimidin-2-yl)carbamate

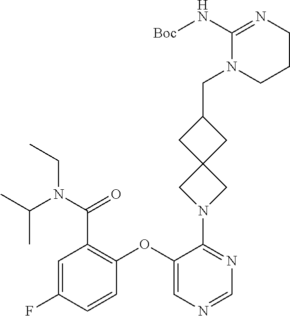 [SMILES: CCN(C(=O)c1cc(F)ccc1Oc1cncnc1N1CC2(CC(CN3CCCN=C3NC(=O)OC(C)(C)C)C2)C1)C(C)C]

This compound was synthesized following the procedure described for the synthesis of Example 17, step 1, starting with 1 equivalent of (2-(5-(2-(ethyl(isopropyl)carbamoyl)-4-fluorophenoxy)pyrimidin-4-yl)-2-azaspiro[3.3]heptan-6-yl)methyl methanesulfonate and 1.2 equivalents of tert-butyl (1,4,5,6-tetrahydropyrimidin-2-yl)carbamate, except that THF was used as the solvent. The reaction was stirred at 70° C. The reaction was quenched with water the mixture was concentrated under reduced pressure. The crude was purified by reverse phase chromatography using a SiliCycle SiliaSep C18 column on Biotage isolera one (Mobile phase A: water, B: ACN) to afford tert-butyl (1-((2-(5-(2-(ethyl(isopropyl)carbamoyl)-4-fluorophenoxy)pyrimidin-4-yl)-2-azaspiro[3.3]heptan-6-yl)methyl)-1,4,5,6-tetrahydropyrimidin-2-yl)carbamate as a solid.

Yield: 14.40%; LCMS (Method D): Rt=2.02 min, m/z=610.2 [M+H]⁺, 61.76%.

### Step 3. 2-((4-(6-((2-Amino-5,6-dihydropyrimidin-1(4H)-yl)methyl)-2-azaspiro[3.3]heptan-2-yl)pyrimidin-S-yl)oxy)-N-ethyl-5-fluoro-N-isopropylbenzamide

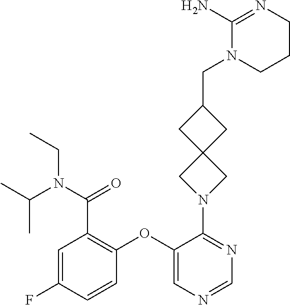 [SMILES: CCN(C(=O)c1cc(F)ccc1Oc1cncnc1N1CC2(CC(CN3CCCN=C3N)C2)C1)C(C)C]

This compound was synthesized following the procedure described for the synthesis of Example 1, step 2, starting with 1 equivalent of tert-butyl (1-((2-(5-(2-(ethyl(isopropyl)carbamoyl)-4-fluorophenoxy)pyrimidin-4-yl)-2-azaspiro[3.3]heptan-6-yl)methyl)-1,4,5,6-tetrahydropyrimidin-2-yl)carbamate, except that 4 equivalents of TMSCl was used. After completion, the reaction was concentrated under reduced pressure. The crude was purified by prep HPLC (Method G), to obtain 2-((4-(6-((2-amino-5,6-dihydropyrimidin-1(4H)-yl)methyl)-2-azaspiro[3.3]heptan-2-yl)pyrimidin-5-yl)oxy)-N-ethyl-5-fluoro-N-isopropylbenzamide as a solid.

Yield: 28.9%; ¹H NMR (400 MHz, DMSO-d₆): δ 8.32-8.22 (m, 1H), 7.81-7.54 (m, 3H), 7.35-7.19 (m, 2H). 7.06-6.90 (m, 1H), 4.46-3.93 (m, 4H), 3.84-3.69 (m, 1H), 3.50-3.38 (m, 2H), 3.27-3.18 (m, 4H), 3.17-3.10 (m, 2H), 2.43-2.37 (m, 1H), 2.28-2.14 (m, 2H), 2.00-1.89 (m, 2H), 1.84-1.75 (m, 2H), 1.23-0.97 (m, 9H); LCMS (Method D): Rt=1.76 min, m/z=510.2 [M+H]⁺; HPLC (Method A): Rt=4.61 min, 98.73%.

### Example 21. N-Ethyl-S-fluoro-N-isopropyl-2-((4-(6-((4,5,6,7-tetrahydro-3H-imidazo[4,5-c]pyridin-3-yl)methyl)-2-azaspiro[3.3]heptan-2-yl)pyrimidin-5-yl)oxy)benzamide (Compound No. 21)

### Example 22. N-Ethyl-5-fluoro-N-isopropyl-2-((4-(6-((4,5,6,7-tetrahydro-1H-imidazo[4,5-c]pyridin-1-yl)methyl)-2-azaspiro[3.3]heptan-2-yl)pyrimidin-5-yl)oxy)benzamide

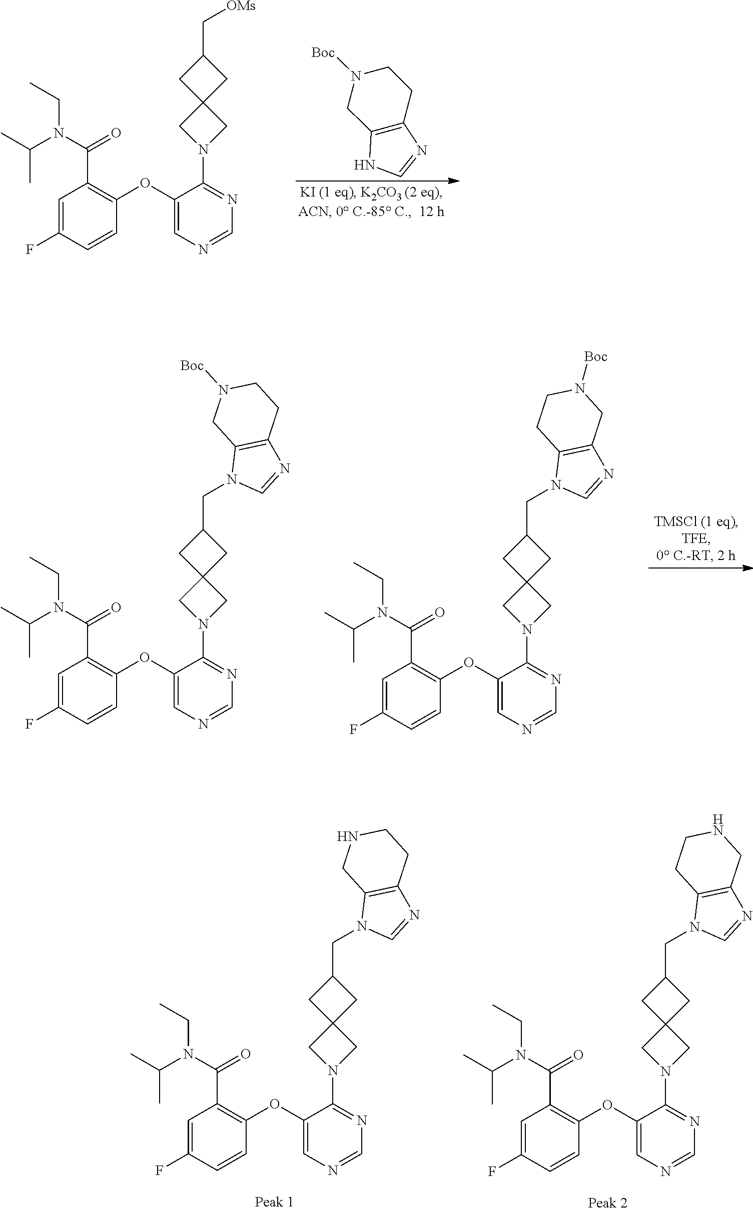 [SMILES: CCN(C(=O)c1cc(F)ccc1Oc1cncnc1N1CC2(CC(COS(C)(=O)=O)C2)C1)C(C)C ; CC(C)(C)OC(=O)N1CCc2nc[nH]c2C1 ; CCN(C(=O)c1cc(F)ccc1Oc1cncnc1N1CC2(CC(Cn3cnc4c3CN(C(=O)OC(C)(C)C)CC4)C2)C1)C(C)C ; CCN(C(=O)c1cc(F)ccc1Oc1cncnc1N1CC2(CC(Cn3cnc4c3CCN(C(=O)OC(C)(C)C)C4)C2)C1)C(C)C ; CCN(C(=O)c1cc(F)ccc1Oc1cncnc1N1CC2(CC(Cn3cnc4c3CNCC4)C2)C1)C(C)C ; CCN(C(=O)c1cc(F)ccc1Oc1cncnc1N1CC2(CC(Cn3cnc4c3CCNC4)C2)C1)C(C)C]

### Step 1. tert-Butyl 3-((2-(5-(2-(ethyl(isopropyl)carbamoyl)-4-fluorophenoxy)pyrimidin-4-yl)-2-azaspiro[3.3]heptan-6-yl)methyl)-3,4,6,7-tetrahydro-5H-imidazo[4,5-c]pyridine-5-carboxylate and tert-butyl 1-((2-(5-(2-(ethyl(isopropyl)carbamoyl)-4-fluorophenoxy)pyrimidin-4-yl)-2-azaspiro[3.3]heptan-6-yl)methyl)-1,4,6,7-tetrahydro-5H-imidazo[4,5-c]pyridine-5-carboxylate

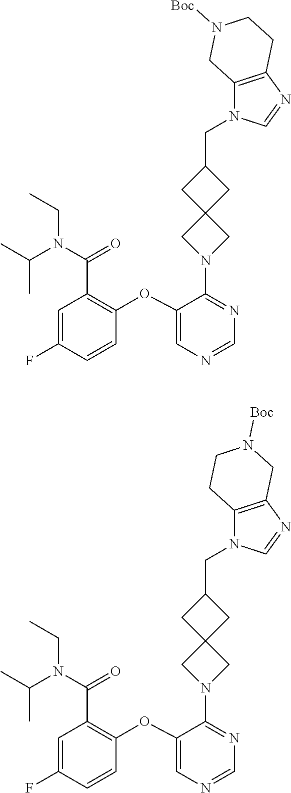 [SMILES: CCN(C(=O)c1cc(F)ccc1Oc1cncnc1N1CC2(CC(Cn3cnc4c3CN(C(=O)OC(C)(C)C)CC4)C2)C1)C(C)C ; CCN(C(=O)c1cc(F)ccc1Oc1cncnc1N1CC2(CC(Cn3cnc4c3CCN(C(=O)OC(C)(C)C)C4)C2)C1)C(C)C]

To a stirred solution of (2-5-(2-(ethyl(isopropyl)carbamoyl)-4-fluorophenoxy)pyrimidin-4-yl)-2-azaspiro[3.3]heptan-6-yl)methyl methanesulfonate (290 mg, 0.572 mmol) and tert-butyl 3,4,6,7-tetrahydro-5H-imidazo[4,5-c]pyridine-5-carboxylate (128 mg, 0.572 mmol) in ACN (3 mL), potassium carbonate (158 mg, 1.145 mmol) and potassium iodide (95 mg, 0.572 mmol) were added. The reaction was heated to 85° C. for 12 h, and monitored by TLC (30% EtOAc in hexane.) After completion, the reaction was quenched with water (25 mL) and extracted with EtOAc (3×25 mL). The combined organic layer was dried over anhydrous sodium sulfate and filtered, and the filtrate was concentrated under reduced pressure to obtain a mixture of regioisomers, tert-butyl 3-((2-(5-(2-(ethyl(isopropyl)carbamoyl)-4-fluorophenoxy)pyrimidin-4-yl)-2-azaspiro[3.3]heptan-6-yl)methyl)-3,4,6,7-tetrahydro-5H-imidazo[4,5-c]pyridine-5-carboxylate and tert-butyl 1-((2-(5-(2-(ethyl(isopropyl)carbamoyl)-4-fluorophenoxy)pyrimidin-4-yl)-2-azaspiro[3.3]heptan-6-yl)methyl)-1,4,6,7-tetrahydro-5H-imidazo[4,5-c]pyridine-5-carboxylate (200 mg, 20.05% yield) as a solid. This material was used without further purification. LCMS (Method D): Rt=1.94 min, m/z=634.2 [M+H]⁺, 40.45%.

### Step 2. N-Ethyl-5-fluoro-N-isopropyl-2-((4-(6-((4,5,6,7-tetrahydro-3H-imidazo[4,5-c]pyridin-3-yl)methyl)-2-azaspiro[3.3]heptan-2-yl)pyrimidin-5-yl)oxy)benzamide and N-ethyl-5-fluoro-N-isopropyl-2-((4-(6-((4,5,6,7-tetrabydro-1H-imidazo[4,5-c]pyridin-1-yl)methyl)-2-azaspiro[3.3]heptan-2-yl)pyrimidin-5-yl)oxy)benzamide

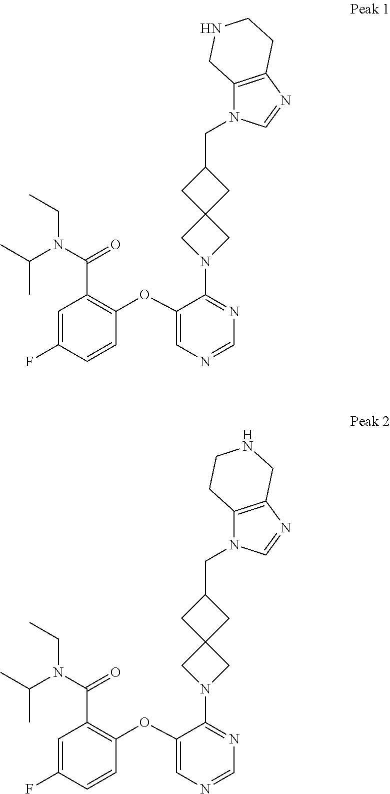 [SMILES: CCN(C(=O)c1cc(F)ccc1Oc1cncnc1N1CC2(CC(Cn3cnc4c3CNCC4)C2)C1)C(C)C ; CCN(C(=O)c1cc(F)ccc1Oc1cncnc1N1CC2(CC(Cn3cnc4c3CCNC4)C2)C1)C(C)C]

In a dried, 25 mL round bottom flask under a nitrogen atmosphere, the regioisomeric mixture of tert-butyl 3-((2-(5-(2-(ethyl(isopropyl)carbamoyl)-4-fluorophenoxy)pyrimidin-4-yl)-2-azaspiro[3.3]heptan-6-yl)methyl)-3,4,6,7-tetrahydro-5H-imidazo[4,5-c]pyridine-5-carboxylate and tert-butyl 1-((2-(5-(2-(ethyl(isopropyl)carbamoyl)-4-fluorophenoxy)pyrimidin-4-yl)-2-azaspiro[3.3]heptan-6-yl)methyl)-1,4,6,7-tetrahydro-5H-imidazo[4,5-c]pyridine-5-carboxylate (58 ng, 0.092 mmol) was dissolved in TFE (10 mL), and TMSCl (9.94 mg, 0.092 mmol) was added at 0° C. The reaction was stirred at RT for 2 h, and monitored by TLC (100% EtOAc). After completion, the reaction was concentrated under reduced pressure. The crude was diluted with sodium bicarbonate solution (25 mL), and the mixture was extracted with 10/MeOH in DCM (3×25 mL), The combined organic layer was dried over anhydrous sodium sulfate and filtered, and the filtrate was concentrated under reduced pressure. The crude was purified by Prep HPLC (Method F) to obtain a mixture of regioisomers, N-ethyl-5-fluoro-N-isopropyl-2-((4-(6-((4,5,6,7-tetrahydro-3H-imidazo[4,5-c]pyridin-3-yl)methyl)-2-azaspiro[3.3]heptan-2-yl)pyrimidin-5-yl)oxy)benzamide and N-ethyl-5-fluoro-N-isopropyl-2-((4-(6-((4,5,6,7-tetrahydro-1H-imidazo[4,5-c]pyridin-1-yl)methyl)-2-azaspiro[3.3]heptan-2-yl)pyrimidin-5-yl)oxy)benzamide (60 mg, quantitative) as a solid. LCMS (Method D): Rt=1.57 min, m/z=534.1 [M+H]⁺; HPLC (Method A): Rt=4.38-4.41 min, 81.57%; Achiral SFC (Method A): Peak 1, Rt=2.36 min, 42.21%; Peak 1, Rt=3.83 min, 40.52%.

This mixture of regioisomers was purified by achiral Prep SFC (Method B) to obtain both isomers.

Isomer 1: N-Ethyl-5-fluoro-V-isopropyl-2-((4-(6-((4,5,6,7-tetrahydro-3H-imidazo[4,5-c]pyridin-3-yl)methyl)-2-azaspiro[3.3]heptan-2-yl)pyrimidin-5-yl)oxy)benzamide (10.42 mg, 19.32% yield)) as a solid. ¹H NMR (400 MHz, DMSO-d₆): δ 8.31-8.23 (m, 1H), 7.79-7.67 (m, 1H), 7.38 (s, 1H), 7.33-7.20 (m, 2H), 7.06-6.95 (m, 1H), 4.52-3.95 (m, 4H), 3.82-3.71 (m, 3H), 3.64 (s, 2H), 3.45-3.37 (m, 1H), 3.28-3.10 (m, 2H), 2.85 (br t, J=5.5 Hz, 2H), 2.46-2.35 (m, 3H), 2.25-2.15 (m, 2H), 1.97-1.87 (m, 2H), 1.22-0.96 (m, 9H); LCMS (Method B): Rt=0.97 min, m/z=534.4 [M+H]⁺; HPLC (Method A): Rt=4.34 min, 98.36%; Achiral SFC (Method A): Rt=2.93 min, 100%.

Isomer 2: N-Ethyl-5-fluoro-N-isopropyl-2-((4-(6-((4,5,6,7-tetrahydro-1H-imidazo[4,5-c]pyridin-1-yl)methyl)-2-azaspiro[3.3]heptan-2-yl)pyrimidin-5-yl)oxy)benzamide (7.3 mg, 13.52% yield)) as a solid. ¹H NMR (400 MHz, DMSO-d₆): δ 8.31-8.23 (m, 1H), 7.79-7.64 (m, 1H), 7.43-7.35 (m, 1H), 7.33-7.20 (m, 2H), 7.06-6.95 (m, 1H), 4.48-3.96 (m, 4H), 3.83-3.70 (m, 3H), 3.58-3.48 (m, 2H), 3.45-3.37 (m, 1H), 3.26-3.07 (m, 2H), 2.89 (br t, J=5.4 Hz, 2H), 2.47-2.37 (m, 3H), 2.27-2.16 (m, 2H), 1.97-1.86 (m, 2H), 1.22-0.96 (m, 9H); LCMS (Method B): Rt=1.04 min, m/z=534.4 [M+H]⁺; HPLC (Method A): Rt=4.40 min, 98.23%; Achiral SFC (Method A): Rt=4.38 min, 100%.

### Example 23. 2-((4-(6-((5,6-Dihydropyrrolo[3,4-d]imidazol-1(4H)-yl)methyl)-2-azaspiro[3.3]heptan-2-yl)pyrimidin-5-yl)oxy)-N-ethyl-S-fluoro-N-isopropylbenzamide (Compound No. 23)

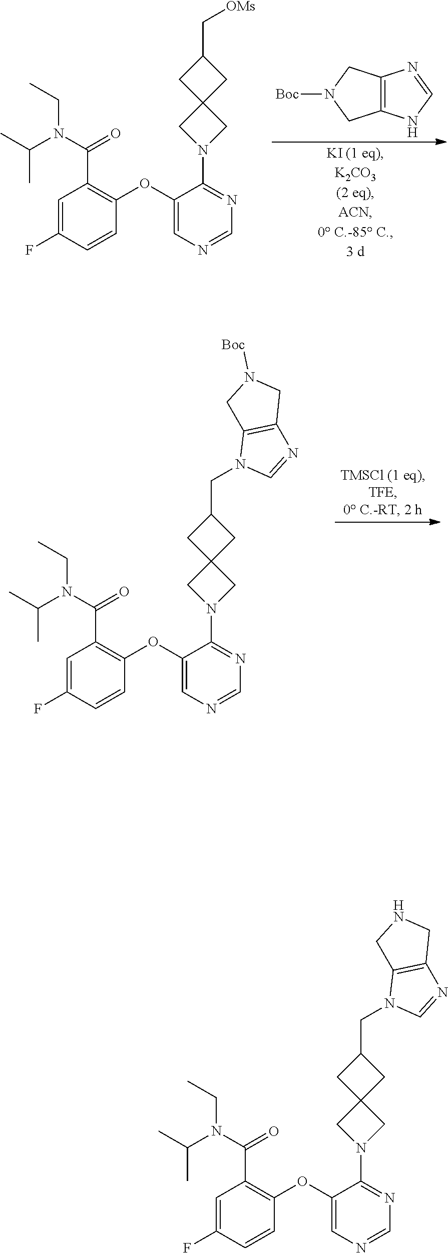 [SMILES: CCN(C(=O)c1cc(F)ccc1Oc1cncnc1N1CC2(CC(COS(C)(=O)=O)C2)C1)C(C)C ; CC(C)(C)OC(=O)N1Cc2nc[nH]c2C1 ; CCN(C(=O)c1cc(F)ccc1Oc1cncnc1N1CC2(CC(Cn3cnc4c3CN(C(=O)OC(C)(C)C)C4)C2)C1)C(C)C ; CCN(C(=O)c1cc(F)ccc1Oc1cncnc1N1CC2(CC(Cn3cnc4c3CNC4)C2)C1)C(C)C]

### Step 1. tert-Butyl 1-((2-(5-(2-(ethyl(isopropyl)carbamoyl)-4-fluorophenoxy)pyrimidin-4-yl)-2-azaspiro[3.3]heptan-6-yl)methyl)-4,6-dihydropyrrolo[3,4-d]imidazole-5(1H)-carboxylate

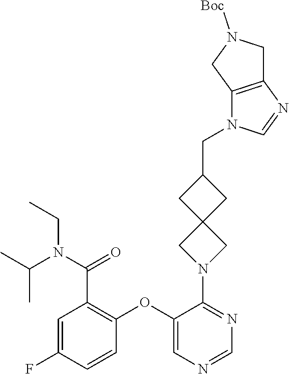 [SMILES: CCN(C(=O)c1cc(F)ccc1Oc1cncnc1N1CC2(CC(Cn3cnc4c3CN(C(=O)OC(C)(C)C)C4)C2)C1)C(C)C]

This compound was synthesized following the procedure described for the synthesis of Example 19, step 1, starting with 1 equivalent of (2-(5-(2-(ethyl(isopropyl)carbamoyl)-4-fluorophenoxy)pyrimidin-4-yl)-2-azaspiro[3.3]heptan-6-yl)methyl methanesulfonate and 1 equivalent of tert-butyl 4,6-dihydropyrrolo[3,4-d]imidazole-5(1H)-carboxylate, except that 1 equivalent of KI and 2 equivalents of K₂CO₃ were used. The reaction was stirred at 85° C. After work-up, the filtrate was concentrated under reduced pressure. The crude compound was purified by silica gel flash column chromatography using EtOAc in hexane (product eluted at 70% EtOAc in hexane) to obtain tert-butyl 1-((2-(5-(2-(ethyl(isopropyl)carbamoyl)-4-fluorophenoxy)pyrimidin-4-yl)-2-azaspiro[3.3]heptan-6-yl)methyl)-4,6-dihydropyrrolo[3,4-d]imidazole-5(1H)-carboxylate as a gum.

Yield: 24.80%; LCMS (Method D): Rt=1.88 min, m/z=620.2 [M+H]⁺, 40.66%.

### Step 2. 2-((4-(6-((5,6-Dihydropyrrolo[3,4-d]imidazol-1(4H)-yl)methyl)-2-azaspiro[3.3]heptan-2-yl)pyrimidin-S-yl)oxy)-N-ethyl-5-fluoro-N-isopropylbenzamide

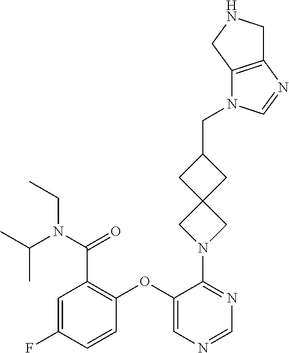 [SMILES: CCN(C(=O)c1cc(F)ccc1Oc1cncnc1N1CC2(CC(Cn3cnc4c3CNC4)C2)C1)C(C)C]

This compound was synthesized following the procedure described for the synthesis of Example 1, step 2, starting with 1 equivalent of tert-butyl 1-((2-(5-(2-(ethyl(isopropyl)carbamoyl)-4-fluorophenoxy)pyrimidin-4-yl)-2-azaspiro[3.3]heptan-6-yl)methyl)-4,6-dihydropyrrolo[3,4-d]imidazole-5(1H)-carboxylate, except that 1 equivalent of TMSCl was used. After completion, the reaction was concentrated under reduced pressure. The crude was diluted with sodium bicarbonate solution and the mixture was extracted with 10% MeOH in DCM. The combined organic layer was dried over anhydrous sodium sulfate and filtered, and the filtrate was concentrated under reduced pressure. The crude was purified by Prep HPLC (Method F) to obtain 2-((4-(6-((5,6-dihydropyrrolo[3,4-d]imidazol-1(4H)-yl)methyl)-2-azaspiro[3.3]heptan-2-yl)pyrimidin-5-yl)oxy)-N-ethyl-5-fluoro-N-isopropylbenzamide as a solid.

Yield: 11.86%; ¹H NMR (400 MHz, DMSO-d₆): δ 8.30-8.22 (m, 1H), 7.79-7.65 (m. 1H), 7.53-7.36 (m, 1H), 7.35-7.21 (m, 2H), 7.06-6.95 (m, 1H), 4.49-4.30 (m, 1H), 4.24-4.06 (m, 4H), 4.04-3.88 (m, 2H), 3.87-3.81 (m, 2H), 3.80-3.62 (m, 2H), 3.46-3.39 (m, 2H), 3.27-3.10 (m, 2H), 2.29-2.13 (m, 2H), 1.98-1.84 (m, 2H), 1.22-0.96 (n, 9H); LCMS (Method B): Rt=1.07 min, m/z=520.2 [M+H]⁺; HPLC (Method A): Rt=4.16 min, 99.56%.

### Example 24. N-Ethyl-5-fluoro-N-isopropyl-2-((5-(6-(((4S,8aS)-octahydro-4H-pyrido[4,3-b][1,4]oxazin-4-yl)methyl)-2-azaspiro[3.3]heptan-2-yl)-1,2,4-triazin-6-yl)oxy)benzamide (Compound No. 24)

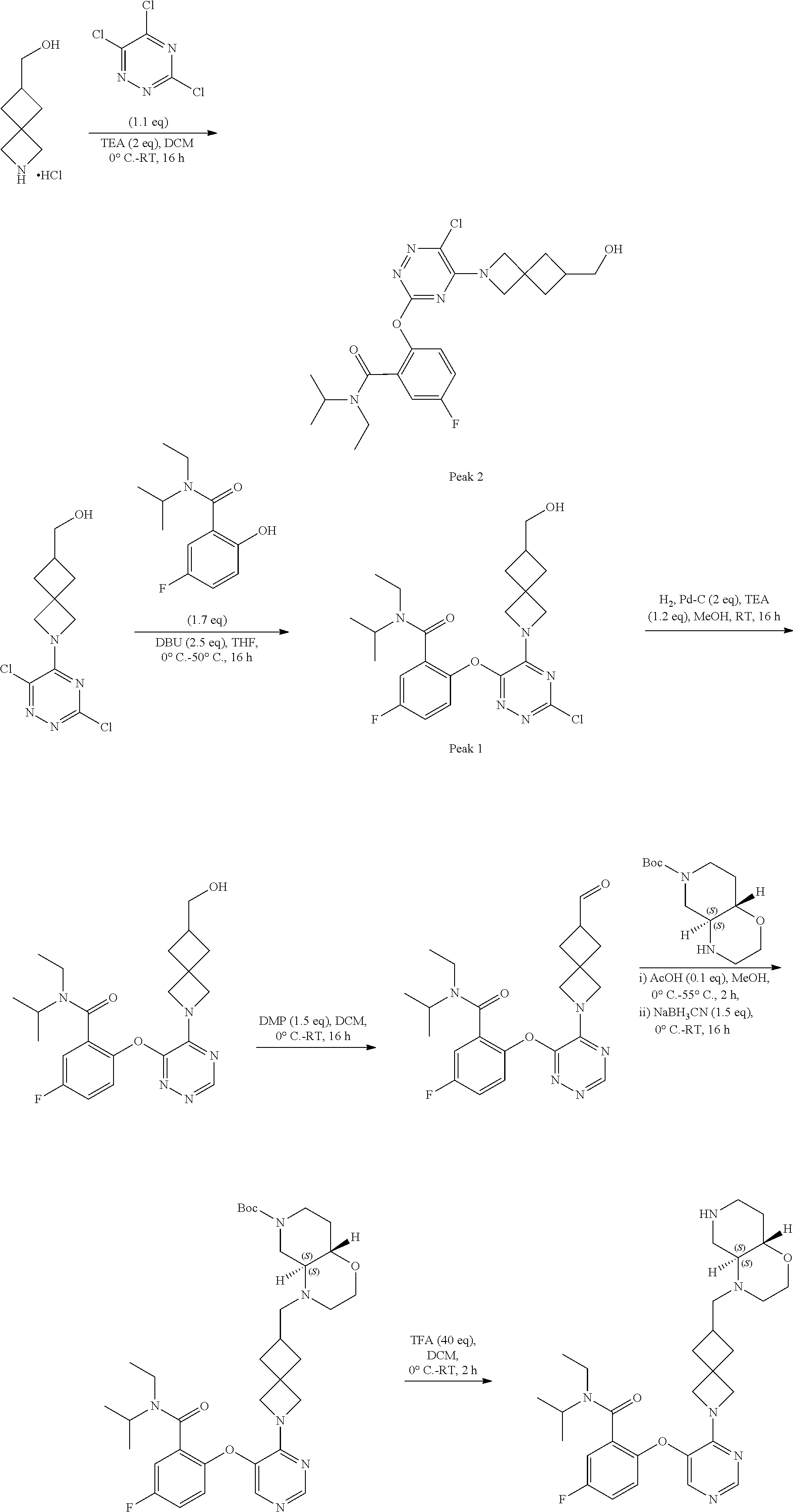

### Step 1. (2-(3,6-Dichloro-1,2,4-triazin-5-yl)-2-azaspiro[3.3]heptan-6-yl)methanol

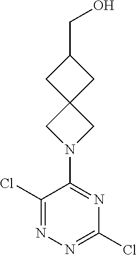 [SMILES: OCC1CC2(C1)CN(c1nc(Cl)nnc1Cl)C2]

To a stirred solution of (2-azaspiro[3.3]heptan-6-yl)methanol hydrochloride (1.1 g, 6.72 mmol) in DCM (20 mL), TEA (1.889 mL, 13.44 mmol) was added. To this reaction mixture, 3,5,6-trichloro-12,4-triazine (1,363 g, 7.39 mmol) was added at 0° C. under a nitrogen atmosphere. The reaction was stirred at RT for 16 h, and monitored by TLC (50% EtOAc in hexane). After completion, the reaction was quenched with water (15 mL), and the mixture was extracted with DCM (4×70 mL). The combined organic layer was washed with aqueous brine solution (30 mL), dried over anhydrous sodium sulfate and filtered, and the filtrate was concentrated under reduced pressure. The residue was purified by silica gel flash column chromatography using EtOAc in hexane (product eluted at 50-60% EtOAc in hexane) to afford (2-(3,6-dichloro-1,2,4-triazin-5-yl)-2-azaspiro[3.3]heptan-6-yl)methanol (I g, 53% yield) as a solid. ¹H NMR (400 MHz, DMSO-d₆): δ 4.75-4.58 (m, 2H), 4.56-4.50 (m, 1H), 4.18-4.02 (m, 2H), 3.36-3.33 (m, 2H), 2.24 (br s, 2H), 2.03-1.93 (m, 2H), one proton merged with solvent peaks; LCMS (Method D): Rt=1.36 min, m/z=274.9 [M+H]⁺, 98.07%.

### Step 2. 2-((3-Chloro-5-(6-(hydroxymethyl)-2-azaspiro[3.3]heptan-2-yl)-1,2,4-triazin-6-yl)oxy)-N-ethyl-5-fluoro-N-isopropylbenzamide and 2-((6-chloro-5-(6-(hydroxymethyl)-2-azaspiro[3.3]heptan-2-yl)-1,2,4-triazin-3-oxy)-N-ethyl-5-fluoro-N-isopropylbenzamide

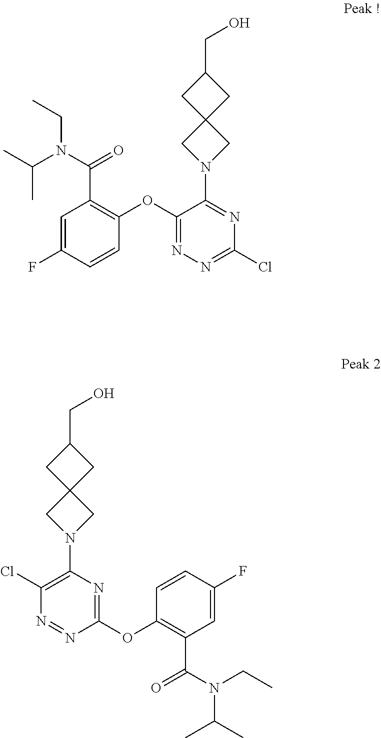 [SMILES: CCN(C(=O)c1cc(F)ccc1Oc1nnc(Cl)nc1N1CC2(CC(CO)C2)C1)C(C)C ; CCN(C(=O)c1cc(F)ccc1Oc1nnc(Cl)c(N2CC3(CC(CO)C3)C2)n1)C(C)C]

To a stirred solution of (2-(3,6-dichloro-1,2,4-triazin-5-yl)-2-azaspiro[3.3]heptan-6-yl)methanol (850 mg, 3.09 mmol) in THF (20 mL), DBU (1.153 mL, 7.72 mmol) and N-ethyl-5-fluoro-2-hydroxy-N-isopropylbenzamide (1183 mg, 5.25 mmol) were added at 0° C. under a nitrogen atmosphere. The reaction was stirred at 50° C. for 16 h, and monitored by TLC (100% EtOAc). After completion, the reaction was quenched with water (100 mL), and the mixture was extracted with ethyl acetate (2×100 mL). The combined organic layer was washed with brine solution (50 mL), dried over anhydrous sodium sulfate and filtered, and the filtrate was concentrated under reduced pressure. The residue was purified by silica gel flash column chromatography using EtOAc in hexane (product eluted at 90 to 100%% EtOAc in hexane) to afford both regioisomers.

Peak 1: 2-((3-Chloro-5-(6-(hydroxymethyl)-2-azaspiro[3.3]heptan-2-yl)-1,2,4-triazin-6-yl)oxy)-N-ethyl-5-fluoro-V-isopropylbenzamide (400 mg, 27.8% yield) as a solid. ¹H NMR (400 MHz, DMSO-d₆): δ 7.49-7.41 (m, 1H), 7.40-7.32 (m, 2H), 4.61-4.45 (m, 3H), 4.35-4.06 (m, 2H), 3.66-3.55 (m, 1H), 3.46-3.39 (m, 1H), 3.18-2.98 (m, 2H), 2.29-2.18 (m, 3H), 1.99-1.93 (m, 2H), 1.17-0.75 (m, 91H), one proton merged with solvent peaks; LCMS (Method D): Rt=1.71 min, m/z=464.1 [M+H]⁺, 99.56%.

Peak 2: 2-((6-Chloro-5-(6-(hydroxymethyl)-2-azaspiro[3.3]heptan-2-yl)-1,2,4-triazin-3-yl)oxy)-N-ethyl-5-fluoro-N-isopropylbenzamide (180 mg, 12.56% yield) as a liquid. LCMS (Method D): Rt=1.72 mint, m/z=464.1 [M+H]⁺, 98.19%.

### Step 3. N-Ethyl-5-fluoro-2-((5-(6-(hydroxymethyl)-2-azaspiro[3.3]heptan-2-yl)-1,2,4-triazin-6-yl)oxy)-N-isopropylbenzamide

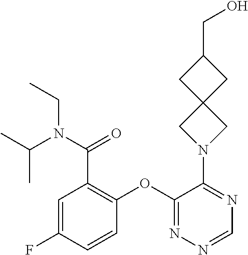 [SMILES: CCN(C(=O)c1cc(F)ccc1Oc1nncnc1N1CC2(CC(CO)C2)C1)C(C)C]

In a dried, round bottom flask under a nitrogen atmosphere, 2-((3-chloro-5-(6-(hydroxymethyl)-2-azaspiro[3.3]heptan-2-yl)-1,2,4-triazin-6-yl)oxy)-N-ethyl-5-fluoro-N-isopropylbenzamide (400 mg, 0.862 mmol) was dissolved in MeOH (20 mL), and TEA (0.145 mL, 1.035 mmol)) was added at RT. Pd—C (184 mg, 0.862 mmol) was then added, and the reaction was stirred at RT for 16 h under a hydrogen atmosphere (balloon pressure). The reaction was monitored by TLC (100% EtOAc). After completion, the reaction was filtered through a Celite® pad, the pad was washed with MeOH (100 mL), and the filtrate was concentrated under reduced pressure. The crude product was purified by silica gel flash column chromatography using MeOH in DCM (product eluted at 2-5% MeOH in DCM) to obtain N-ethyl-5-fluoro-2-((5-(6-(hydroxymethyl)-2-azaspiro[3.3]heptan-2-yl)-1,2,4-triazin-6-yl)oxy)-N-isopropylbenzamide (200 mg, 45.8% yield) as a solid. ¹H NMR (400 MHz, DMSO-d₆): δ 8.44 (s, 1H), 7.48-7.41 (m, 1H), 7.39-7.32 (m, 2H), 4.58-4.38 (m, 3H), 4.18-4.08 (m, 2H), 3.68-3.57 (m, 1H), 3.15-2.97 (m, 1H), 2.29-2.14 (m, 3H), 2.00-1.93 (m, 2H), 1.15-0.67 (m, 9H), three protons merged with solvent peaks; LCMS (Method D): Rt=1.47 min, m/z=430.2 [M+H]⁺, 84.89%.

### Step 4. N—-Ethyl-5-fluoro-2-((5-(6-formyl-2-azaspiro[3.3]heptan-2-yl)-1,2,4-triazin-6-yl)oxy)-N-isopropylbenzamide

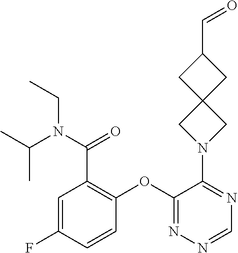 [SMILES: CCN(C(=O)c1cc(F)ccc1Oc1nncnc1N1CC2(CC(C=O)C2)C1)C(C)C]

This compound was synthesized following the procedure described for the synthesis of Intermediate 12, starting with 1 equivalent of N-ethyl-5-fluoro-2-((5-(6-(hydroxymethyl)-2-azaspiro[3.3]heptan-2-yl)-1,2,4-triazin-6-yl)oxy)-N-isopropylbenzamide. After work-up, organic layer was dried over anhydrous sodium sulfate and filtered, and the filtrate was concentrated under reduced pressure to obtain crude N-ethyl-5-fluoro-2-((5-(6-formyl-2-azaspiro[3.3]heptan-2-yl)-1,2,4-triazin-6-yl)oxy)-N-isopropylbenzamide as a solid. This material was used without further purification.

Yield: 72.4%; LCMS (Method B): Rt=1.45 min, m/z=428.2 [M+H]⁺, 92.70%.

### Step 5. tert-Butyl (4aS,8aS)-4-((2-(6-(2-(ethyl(isopropyl)carbamoyl)-4-fluorophenoxy)-1,2,4-triazin-5-yl)-2-azaspiro[3.3]heptan-6-yl)methyl)hexahydro-2H-pyrido[4,3-b][1,4]oxazine-6(5H)-carboxylate

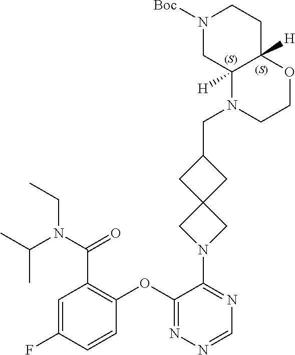

This compound was synthesized following the procedure described for the synthesis of Example 3, step 1, starting with 1 equivalent of N-ethyl-5-fluoro-2-((5-(6-formyl-2-azaspiro[3.3]heptan-2-yl)-1,2,4-triazin-6-yl)oxy)-N-isopropylbenzamide and 1 equivalent of tert-butyl (4aS,8aS)-hexahydro-2H-pyrido[4,3-b][1,4]oxazine-6(5H)-carboxylate. After completion, the reaction was concentrated under reduced pressure, the crude was diluted with water and extracted with DCM. The combined organic layer was dried over anhydrous sodium sulfate and filtered, and the filtrate was concentrated under reduced pressure to afford crude tert-butyl (4aS,8aS)-4-((2-(6-(2-(ethyl(isopropyl)carbamoyl)-4-fluorophenoxy)-1,2,4-triazin-5-yl)-2-azaspiro[3.3]heptan-6-yl)methyl)hexahydro-2H-pyrido[4,3-b][1,4]oxazine-6(5H)-carboxylate as a liquid. This material was used without further purification.

Yield: 44.3%; LCMS (Method D): Rt=2.08 min, m/z=654.4 [M+H]⁺, 63.22%.

### Step 6. N-Ethyl-5-fluoro-N-isopropyl-2-((5-(6-(((4aS,8aS)-octahydro-4H-pyrido[4,3-b][1,4]oxazin-4-yl)methyl)-2-azaspiro[3.3]heptan-2-yl)-1,2,4-triazin-6-yl)oxy)benzamide

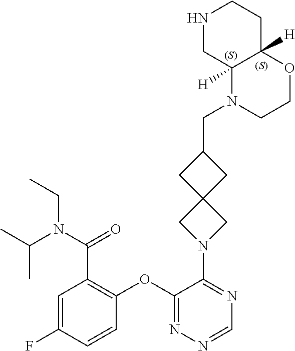

This compound was synthesized following the procedure described for the synthesis of Example 2, step 1, starting with 1 equivalent of tert-butyl (4aS,8aS)-4-((2-(6-(2-(ethyl(isopropyl)carbamoyl)-4-fluorophenoxy)-1,2,4-triazin-5-yl)-2-azaspiro[3.3]heptan-6-yl)methyl)hexahydro-2H-pyrido[4,3-b][1,4]oxazine-6(5H)-carboxylate, except that 40 equivalents of TFA was used. After completion, the reaction was concentrated under reduced pressure. The crude was purified by Prep HPLC (Method 0) to afford N-ethyl-5-fluoro-N-isopropyl-2-((5-(6-(((4aS,8aS)-octahydro-4H-pyrido[4,3-b][1,4]oxazin-4-yl)methyl)-2-azaspiro[3.3]heptan-2-yl)-1,2,4-triazin-6-yl)oxy)benzamide as a solid.

Yield: 31.9%; ¹H NMR (400 MHz, DMSO-d₆): δ 8.44 (s, 1H), 7.48-7.41 (m, 1H), 7.39-7.31 (m, 2H), 4.61-4.24 (m, 2H), 4.21-3.94 (m, 2H), 3.74-3.68 (m, 1H), 3.60 (spt, J=6.5 Hz, 1H), 3.55-3.46 (m, 2H), 3.44-3.41 (m, 1H), 3.23-3.07 (m, 2H), 3.05-2.98 (m, 2H), 2.93-2.80 (m, 1H), 2.65-2.58 (m, 1H), 2.44-2.40 (m, 1H), 2.31-2.00 (m, 5H), 1.92-1.76 (m, 4H), 1.67-1.57 (m, 1H), 1.37-1.24 (m, 1H), 1.16-0.65 (m, 9H); LCMS (Method D): Rt=1.44 min, m/z=554.1 [M+H]+; HPLC (Method A): Rt=4.17 min, 96.51%; Chiral SFC (Method AF): Rt=3.24 min, 100%.

### Example 25. N-Ethyl-5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyridazin-3-yl)oxy)benzamide (Compound No. 25)

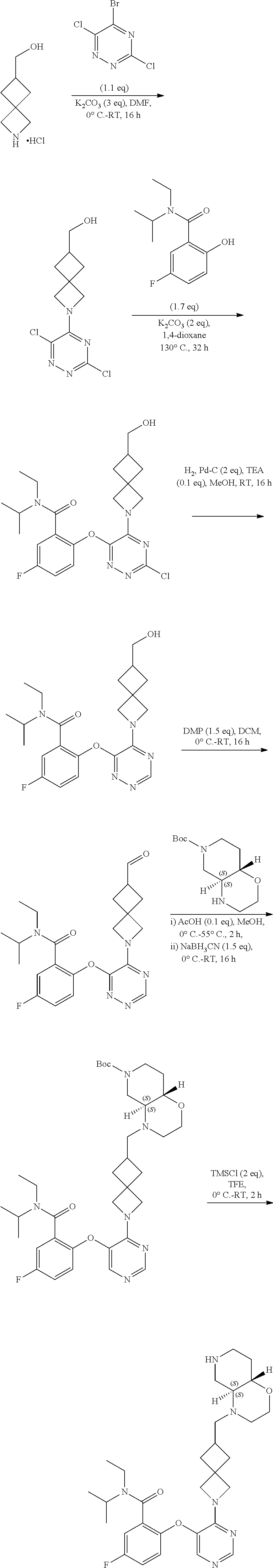

### Step 1. (2-(3,6-Dichloropyridazin-4-yl)-2-azaspiro[3.3]heptan-6-yl)methanol

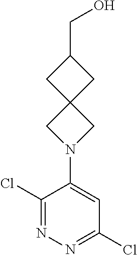 [SMILES: OCC1CC2(C1)CN(c1cc(Cl)nnc1Cl)C2]

In a dried, 50 mL round bottom flask under a nitrogen atmosphere, 4-bromo-3,6-dichloropyridazine (2 g, 8.78 mmol) and (2-azaspiro[3.3]heptan-6-yl)methanol (1.116 g, 8.78 mmol) were dissolved in DMF (20 mL). To this reaction mixture, K₂CO (3.64 g, 26.3 mmol) was added at 0° C., and the reaction was stirred at RT for 16 h. The reaction was monitored by TLC (50% EtOAc in hexane). After completion, the reaction was added to ice cold water (50 mL) and the precipitate was filtered. The solid was purified by silica gel flash column chromatography using EtOAc in hexane (product eluted at 70% EtOAc in hexane) to obtain (2-(3,6-dichloropyridazin-4-yl)-2-azaspiro[3.3]heptan-6-yl)methanol (1 g, 37.6% yield) as a solid. ¹H NMR (400 MHz, DMSO-d₆): δ 6.67 (s, 1H), 4.53 (t, J=5.3 Hz, 1H), 4.36-4.06 (m, 4H), 3.35-3.33 (m, 2H), 2.26-2.18 (m, 3H), 2.01-1.92 (m, 2H); LCMS (Method B): Rt=1.87 min, m/z=274.1 [M+H]⁺, 90.05%.

### Step 2. 2-((6-Chloro-4-(6-(hydroxymethyl)-2-azaspiro[3.3]heptan-2-yl)pyridazin-3-yl)oxy)-N-ethyl-5-fluoro-N-isopropylbenzamide

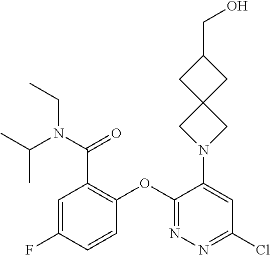 [SMILES: CCN(C(=O)c1cc(F)ccc1Oc1nnc(Cl)cc1N1CC2(CC(CO)C2)C1)C(C)C]

In a dried, 50 mL round bottom flask under a nitrogen atmosphere, (2-(3,6-dichloropyridazin-4-yl)-2-azaspiro[3.3]heptan-6-yl)methanol (I g, 3.65 mmol) was dissolved in 1,4-dioxane (5 mL). To this solution, N-ethyl-5-fluoro-2-hydroxy-N-isopropylbenzamide (0.822 g, 3.65 mmol) and K₂CO. (1.008 g, 7.30 mmol) were added. The reaction was purged with a nitrogen gas for 5 min. The reaction was stirred at 130° C. for 32 h, and monitored by TLC (50% EtOAc in hexane). After completion, the reaction was concentrated under reduced pressure, quenched with ice water (100 mL), and extracted with EtOAc (2×50 mL). The combined organic layer was washed with brine solution (100 mL), dried over anhydrous sodium sulfate and filtered, and the filtrate was concentrated under reduced pressure. The crude material was purified by silica gel flash column chromatography using EtOAc in hexane (product eluted at 100% EtOAc) to afford 2-((6-chloro-4-(6-(hydroxymethyl)-2-azaspiro[3.3]heptan-2-yl)pyridazin-3-yl)oxy)-N-ethyl-5-fluoro-N-isopropylbenzamide (1.1 g, 59.8% yield) as a solid. ¹H NMR (400 MHz, DMSO-d₄): δ 7.41-7.28 (m, 3H), 6.51 (s, 1H), 4.52 (t, J=5.3 Hz, 1H), 4.32-4.06 (m, 4H), 3.68-3.56 (m, 1H), 3.45-3.37 (m, 1H), 3.15-2.98 (m, 1H), 2.27-2.16 (m, 3H), 1.99-1.90 (m, 2H), 1.15-0.64 (m, 9H), two protons merged with solvent peaks; LCMS (Method B): Rt=2.23 min, m/z=463.2 [M+H]⁺, 91.89%.

### Step 3. N-Ethyl-5-fluoro-2-((4-(6-(hydroxymethyl)-2-azaspiro[3.3]heptan-2-yl)pyridazin-3-yl)oxy)-N-isopropylbenzamide

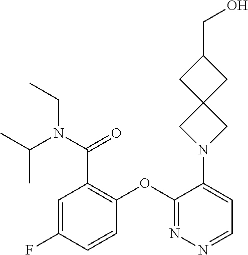 [SMILES: CCN(C(=O)c1cc(F)ccc1Oc1nnccc1N1CC2(CC(CO)C2)C1)C(C)C]

This compound was synthesized following the procedure described for the synthesis of Example 24, step 3, starting with 1 equivalent of 2-((6-chloro-4-(6-(hydroxymethyl)-2-azaspiro[3.3]heptan-2-yl)pyridazin-3-yl)oxy)-N-ethyl-5-fluoro-N-isopropylbenzamide, except that 0.1 equivalent of TEA and 1 equivalent of Pd—C were used. After completion, the reaction was filtered through a Celite® pad, the pad was washed with MeOH, and the filtrate was concentrated under reduced pressure. The crude material was purified by silica gel flash column chromatography using EtOAc in hexane (product eluted 80 to 100% EtOAc in hexane) to afford N-ethyl-5-fluoro-2-((4-(6-(hydroxymethyl)-2-azaspiro[3.3]heptan-2-yl)pyridazin-3-yl)oxy)-N-isopropylbenzamide as a liquid.

Yield: 87%; ¹H NMR (400 MHz, DMSO-d₆): δ 8.39 (d, J=5.4 Hz, 1H), 7.36-7.26 (m, 31H), 6.42 (d, J=5.5 Hz, 1H), 4.52 (t, J=5.3 Hz, 1H), 4.34-3.98 (m, 4H), 3.66 (spt, J=6.6 Hz, 1H), 3.43-3.36 (m, 1H), 3.14-3.02 (m, 2H), 2.29-2.13 (m, 3H), 1.99-1.90 (m, 2H), 1.14-0.62 (m, 9H), one proton merged with solvent peaks; LCMS (Method D): Rt=1.52 min, m/z=429.6 [M+H]⁺, 98.89%.

### Step 4. N-Ethyl-5-fluoro-2-((4-(6-formyl-2-azaspiro[3.3]heptan-2-yl)pyridazin-3-yl)oxy)-N-isopropylbenzamide

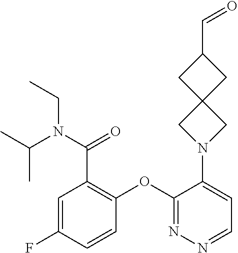 [SMILES: CCN(C(=O)c1cc(F)ccc1Oc1nnccc1N1CC2(CC(C=O)C2)C1)C(C)C]

This compound was synthesized following the procedure described for the synthesis of Intermediate 12, starting with 1 equivalent of N-ethyl-5-fluoro-2-((4-(6-(hydroxymethyl)-2-azaspiro[3.3]heptan-2-yl)pyridazin-3-yl)oxy)-N-isopropylbenzamide. After work-up, organic layer was dried over anhydrous sodium sulfate and filtered, and the filtrate was concentrated under reduced pressure to obtain crude N-ethyl-5-fluoro-2-((4-(6-formyl-2-azaspiro[3.3]heptan-2-yl)pyridazin-3-yl)oxy)-N-isopropylbenzamide as a liquid. This material was used without further purification.

Yield: 44.6%; LCMS (Method B): Rt=1.60 min, m/z=427.3 [M+H]⁺, 88.82%.

### Step 5. tert-Butyl (4aS,8aS)-4-((2-(3-(2-(ethyl(isopropyl)carbamoyl)-4-fluorophenoxy)pyridazin-4-yl)-2-azaspiro[3.3]heptan-6-yl)methyl)hexahydro-2H-pyrido[4,3-b][1,4]oxazine-6(5H)-carboxylate

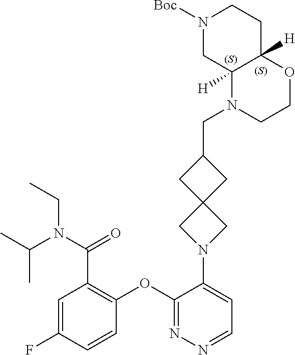

This compound was synthesized following the procedure described for the synthesis of Example 3, step 1, starting with 1 equivalent of N-ethyl-5-fluoro-2-((4-(6-formyl-2-azaspiro[3.3]heptan-2-yl)pyridazin-3-yl)oxy)-N-isopropylbenzamide and 1 equivalent of tert-butyl (4aS,8aS)-hexahydro-2H-pyrido[4,3-b][1,4]oxazine-6(5H)-carboxylate. After completion, the reaction was concentrated under reduced pressure, the crude was dissolved in water, and the mixture was extracted with DCM. The combined organic layer was dried over anhydrous sodium sulfate and filtered, and the filtrate was concentrated under reduced pressure to afford crude tert-butyl (4aS,8aS)-4-((2-(3-(2-(ethyl(isopropyl)carbamoyl)-4-fluorophenoxy)pyridazin-4-yl)-2-azaspiro[3.3]heptan-6-yl)methyl)hexahydro-2H-pyrido[4,3-b][1,4]oxazine-6(5H)-carboxylate as a liquid. This material was used without further purification.

Yield: 43.5%; LCMS (Method B): Rt=1.64 min, m/z=653.4 [M+H]⁺, 85.30%.

### Step 6. N-Ethyl-S-fluoro-isopropyl-2-((4-(6-(((4aS,8aS)-octahydro-4H-pyrido[4,3-b][1,4]oxazin-4-yl)methyl)-2-azaspiro[3.3]heptan-2-yl)pyridazin-3-yl oxy)benzamide

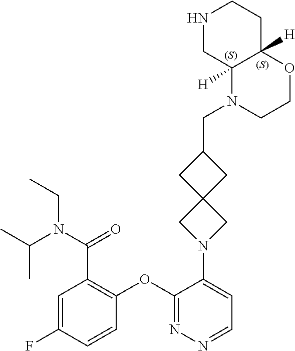

This compound was synthesized following the procedure described for the synthesis of Example 1, step 2, starting with 1 equivalent of tert-butyl (4aS,8aS)-4-((2-(3-(2-(ethyl(isopropyl)carbamoyl)-4-fluorophenoxy)pyridazin-4-yl)-2-azaspiro[3.3]heptan-6-yl)methyl)hexahydro-2H-pyrido[4,3-b][1,4]oxazine-6(5H)-carboxylate. After completion, the reaction was concentrated under reduced pressure, and the residue was triturated with EtOAc. The organic layer was decanted and the crude was purified by Prep HPLC (Method F) to afford N-ethyl-5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyridazin-3-yl)oxy)benzamide as a solid.

Yield: 14.44%; ¹H NMR (400 MHz, DMSO-d₆): δ8.39 (d, J=5.4 Hz, 1H), 7.35-7.24 (m, 3H), 6.41 (d, J=5.5 Hz, 1H), 4.34-3.95 (m, 4H), 3.74-3.62 (m, 2H), 3.55-3.47 (m, 1H), 3.45-3.37 (m, 1H), 3.26-3.16 (m, 1H), 3.13-2.99 (m, 2H), 2.94-2.84 (m, 1H), 2.71-2.59 (m, 2H), 2.48-2.43 (m, 1H), 2.32-2.14 (m, 4H), 2.13-2.04 (m, 2H), 1.90-1.79 (m, 3H), 1.70-1.60 (m, 1H), 1.39-1.24 (m, 1H), 1.14-0.60 (m, 9H), one proton merged with solvent peaks; LCMS (Method D): Rt=1.48 min, m/z=553.2 [M+H]⁺; HPLC (Method F): Rt=4.26 min, 98.69%; Chiral SFC (Method AG): Rt=6.24 min, 100%.

### Example 26. (S)-2-((4-(6-((3-(Aminomethyl)morpholino)methyl)-2-azaspiro[3,3]heptan-2-yl)pyrimidin-5-yl)oxy)-N-ethyl-5-fluoro-N-isopropylbenzamide (Compound No. 26)

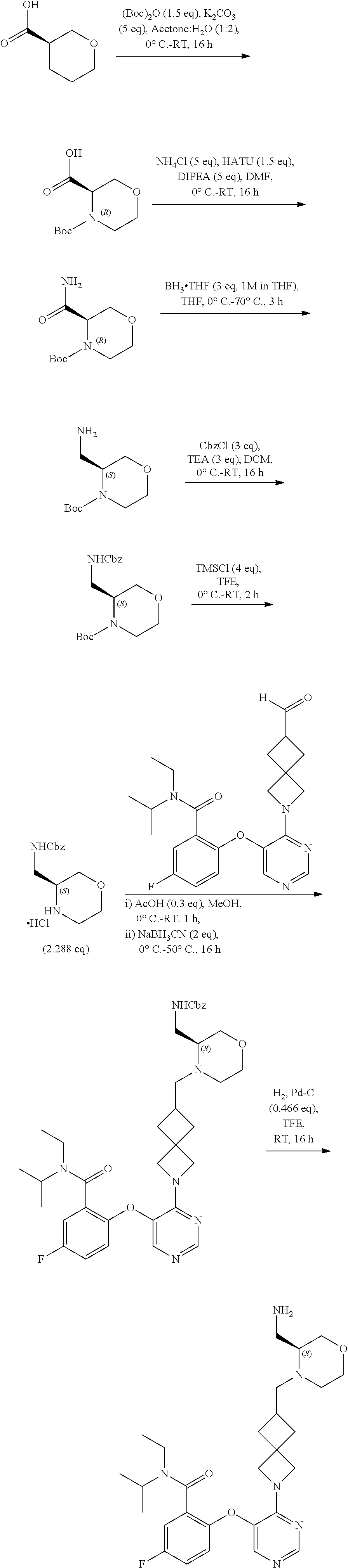

### Step 1. (R)-4-(tert-Butoxycarbonyl)morpholine-3-carboxylic acid

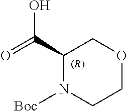

In a dried, 25 mL round bottom flask under a nitrogen atmosphere, (R)-morpholine-3-carboxylic acid hydrochloride (0.5 g, 2.98 mmol) was dissolved in acetone (5 mL), and a solution of K₂CO₃ (2.062 g, 14.92 mmol) in water (10 mL) was added. The reaction was cooled to 0° C., then (Boc)₂O (1.039 mL, 4.48 mmol) was added. The reaction was stirred at RT for 16 h, and monitored by TLC (10% MeOH in DCM). After completion, the reaction was concentrated under reduced pressure to remove the acetone, and the resulting aqueous mixture was washed with DCM (10 mL). The DCM washings were discarded. The aqueous layer was acidified with 1.5 N aqueous HCl (pH~5), and the mixture was extracted with DCM (20 mL). The combined organic layer was dried over anhydrous sodium sulfate and filtered, and the filtrate was concentrated under reduced pressure to afford crude (R)-4-(tert-butoxycarbonyl)morpholine-3-carboxylic acid (0.605 g, 88% yield) as a solid. This material was used without further purification. ¹H NMR (400 MHz, DMSO-d₆): δ 13.05 (br s, 1H), 4.37-4.25 (m, 1H), 4.22-4.09 (m, 1H), 3.87-3.73 (m, 1H), 3.61-3.47 (m, 2H), 3.43-3.26 (m, 1H), 3.24-2.94 (m, 1H), 1.46-1.26 (m, 9H); LCMS-ELSD (Method D): Rt=0.89 min, m/z=230.1 [M−H]⁻, 99.94%.

### Step 2. tert-Butyl (R)-3-carbamoylmorpholine-4-carboxylate

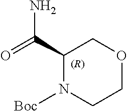

In a 500 mL round bottom flask under a nitrogen atmosphere, (R)-4-(tert-butoxycarbonyl)morpholine-3-carboxylic acid (4 g, 17.30 mmol) was dissolved in DMF (50 mL). Ammonium chloride (4.63 g, 86 mmol), DIPEA (11.18 g, 86 mmol) and HATU (9.87 g, 25.9 mmol) were added at RT. The reaction was stirred at RT for 16 h, and monitored by TLC (10% MeOH in DCM). After completion, the reaction was quenched with water (100 mL), and the mixture was extracted with EtOAc (2×100 mL). The combined organic layer was dried over anhydrous sodium sulfate and filtered, and the filtrate was concentrated under reduced pressure to obtain crude tert-butyl (R)-3-carbamoylmorpholine-4-carboxylate (7 g, quantitative yield). This material was used without further purification. LCMS-ELSD (Method D): Rt=1.24 min, m/z=131.2 [M+H−100]⁺, 76.23%.

### Step 3. tert-Butyl (S)-3-(aminomethyl)morpholine-4-carboxylate

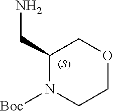

In a dried, 100 mL two neck round bottom flask under a nitrogen atmosphere, tert-butyl (R)-3-carbamoylmorpholine-4-carboxylate (1 g, 4.34 mmol) was dissolved in THE (10 mL). BH₃·THF (13.03 mL, 13.03 mmol, IM in THF) was added at 0° C. The reaction was stirred at 70° C. for 3 h, and monitored by TLC (10/a MeOH in DCM). After completion, the reaction was quenched with MeOH (30 mL) and stirred at 70° C. for 1 h. The reaction was then cooled to RT, water (50 mL) was added, and the mixture was extracted with EtOAc (2×30 mL). The EtOAc layer was discarded. The aqueous layer was basified with saturated sodium bicarbonate (50 mL) and extracted with EtOAc (2×30 mL). The combined organic layer was dried over anhydrous sodium sulfate and filtered, and the filtrate was concentrated under reduced pressure to obtain crude tert-butyl (S)-3-(aminomethyl)morpholine-4-carboxylate (190 mg, 20.23% yield) as a gum. This material was used without further purification.

### Step 4. tert-Butyl (S)-3-(((((benzyloxy)carbonyl)amino)methyl)morpholine-4-carboxylate

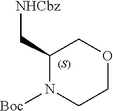

In a dried, 50 mL two neck round bottom flask under a nitrogen atmosphere, tert-butyl (S)-3-(aminomethyl)morpholine-4-carboxylate (190 mg, 0.878 mmol) was dissolved in DCM (10 mL). Cbz-Cl (0.213 mL, 1.493 mmol) and TEA (0.367 mL, 2.64 mmol) were added at 0° C. The reaction was stirred at RT for 16 h, and monitored by TLC (10% MeOH in DCM). After completion, the reaction was quenched with ice cold water (30 mL) and extracted with EtOAc (2×20 mL). The combined organic layer was dried over anhydrous sodium sulfate and filtered, and the filtrate was concentrated under reduced pressure. The residue (0.5 g) was purified by silica gel flash column chromatography using MeOH in DCM (product eluted 2% MeOH in DCM) to obtain tert-butyl (S)-3-((((benzyloxy)carbonyl)amino)methyl)morpholine-4-carboxylate (140 mg, 45.3% yield) as a gum. LCMS-ELSD (Method H): Rt=1.89 min, m/z=251.0 [M+H−100]⁺, 99.59%.

### Step 5. Benzyl (S)-(morpholin-3-ylmethylcarbamate hydrochloride

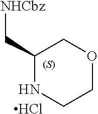

In a 25 mL round bottom flask under a nitrogen atmosphere, tert-butyl (S)-3-((((benzyloxy)carbonyl)amino)methyl)morpholine-4-carboxylate (140 mg, 0.400 mmol) was dissolved in 2,2,2-trifluoroethanol (3 mL). TMS-Cl (0.204 mL, 1.598 mmol) was added at 0° C. The reaction was stirred at RT for 1 h, and monitored by TLC (100% EtOAc). After completion, the reaction was concentrated under reduced pressure to obtain crude benzyl (S)-(morpholin-3-ylmethyl)carbamate hydrochloride (130 mg, 99% yield) as a gum. This material was used without further purification. LCMS (Method D): Rt=1.28 min, m/z=251.2 [M+H]⁺, 53.68%.

### Step 6. Benzyl (S)-((4-((2-(5-(2-(ethyl(isopropyl)carbamoyl)-4-fluorophenoxy)pyrimidin-4-yl)-2-azaspiro[3.3]heptan-6-yl)methyl)morpholin-3-yl)methyl)carbamate

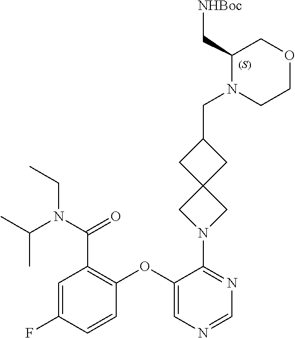

In a dried, 25 mL round bottom flask under a nitrogen atmosphere, N-ethyl-5-fluoro-2-((4-(6-formyl-2-azaspiro[3.3]heptan-2-yl)pyrimidin-5-yl)oxy)-N-isopropylbenzamide (0.13 g, 0.305 mmol) and benzyl (S)-(morpholin-3-ylmethyl)carbamate hydrochloride (0.2 g, 0.697 mmol) were dissolved in MeOH (10 mL). AcOH (5.23 μL, 0.091 mmol) was then added at 0° C., and the reaction was stirred at RT for 1 h. To this reaction mixture, sodium cyanoborohydride (0.038 g, 0.610 mmol) was added at 0° C. The reaction was stirred at 50° C. for 16 h, and monitored by TLC (10% MeOH in DCM). After completion, the reaction was concentrated under reduced pressure. The crude was dissolved in 10% MeOH in DCM (20 mL) and washed with water (20 mL). The organic layer was dried over anhydrous sodium sulfate and filtered, and the filtrate was concentrated under reduced pressure. The residue was purified by Prep HPLC (Method G) to afford benzyl (S)-((4-((2-(5-(2-(ethyl(isopropyl)carbamoyl)-4-fluorophenoxy)pyrimidin-4-yl)-2-azaspiro[3.3]heptan-6-yl)methyl)morpholin-3-yl)methyl)carbamate (0.08 g, 38.9% yield) as a semi-solid. ¹H NMR (400 MHz, DMSO-d₆): δ 8.31-8.21 (m, 1H), 7.78-7.61 (m, 1H), 7.41-7.16 (m, 8H), 7.08-6.96 (m, 1H), 5.01 (s, 2H), 4.22-3.93 (m, 5H), 3.81-3.70 (m, 1H), 3.61-3.36 (m, 411), 3.29-3.20 (m, 2H), 3.05-2.92 (m, 1H), 2.65-2.55 (m, 2H), 2.32-2.13 (m, 5H), 1.90-1.76 (m, 2H), 1.24-0.93 (m, 9H), one proton merged with solvent peaks; LCMS (Method D): Rt=2.11 min, m/z=661.4 [M+H]⁺, 97.94%.

### Step 7. (S)-2-((4-(6-((3-(Aminomethyl)morpholino)methyl)-2-azaspiro[3.3]heptan-2-yl)pyrimidin-5-yl)oxy)-N-ethyl-5-fluoro-N-isopropylbenzamide

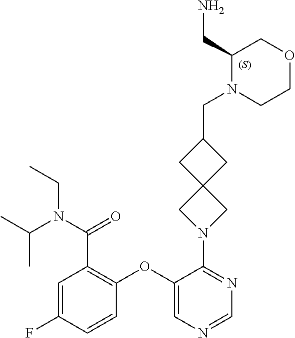

In a dried, 50 mL round bottom flask, benzyl (S)-((4-((2-(5-(2-(ethyl(isopropyl)carbamoyl)-4-fluorophenoxy)pyrimidin-4-yl)-2-azaspiro[3.3]heptan-6-yl)methyl)morpholin-3-yl)methyl)carbamate (80 mg, 0.121 mmol) was dissolved in TFE (5 mL), and Pd—C (60 mg, 0.056 mmol) was added at RT. The reaction was stirred under a hydrogen atmosphere (balloon pressure) at RT for 16 h, and monitored by TLC (10% MeOH in DCM). After completion, the reaction was filtered through a Celite® pad, the pad was washed with MeOH, and the filtrate was concentrated under reduced pressure. The residue (60 mg) was purified by prep HPLC (Method N) to afford (S)-2-((4-(6-((3-(aminomethyl)morpholino)methyl)-2-azaspiro[3.3]heptan-2-yl)pyrimidin-5-yl)oxy)-N-ethyl-5-fluoro-N-isopropylbenzamide (20 mg, 30.8% yield) as a semi-solid. ¹H NMR (400 MHz, DMSO-d₆): δ 8.34-8.19 (m, 1H), 7.85-7.65 (m, 1H), 7.34-7.19 (m, 2H), 7.07-6.91 (m, 1H), 4.47-3.93 (m, 4H), 3.75 (spt, J=6.5, 1H), 3.70-3.56 (m, 2H), 3.53-3.41 (m, 3H), 2.91-2.77 (m, 2H), 2.76-2.60 (m, 2H), 2.44-2.39 (m, 2H), 2.33-2.16 (m, 4H), 1.89-1.80 (m, 2H), 1.27-0.94 (m, 9H), three protons merged with solvent peak; LCMS (Method F): Rt=0.99 min, m/z 527.2 [M+H]⁺; RPLC (Method A): Rt=4.27 min, 98.35%; Chiral SFC (Method AH): Rt=2.98 min, 98.26%.

### Example 27. (R)-2-((4-(6-((3-(Aminomethyl)morpholino)methyl)-2-azaspiro[3.3]heptan-2-yl)pyrimidin-5-yl)oxy)-N-ethyl-5-fluoro-N-isopropylbenzamide (Compound No. 27)

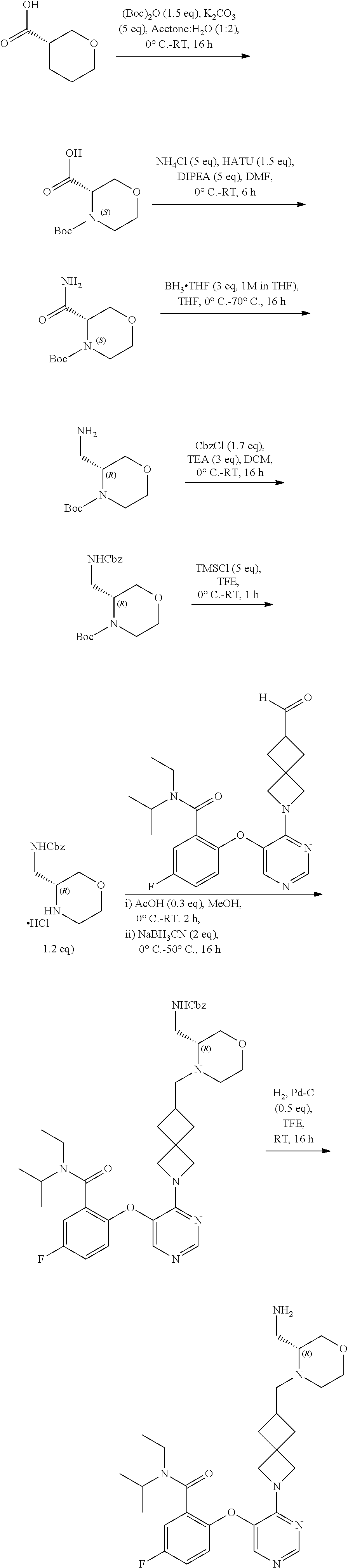

### Step 1. (S)-4-(tert-Butoxycarbonyl)morpholine-3-carboxylic acid

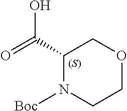

This compound was synthesized following the procedure described for the synthesis of Example 26, step 1, starting with 1 equivalent of(S)-morpholine-3-carboxylic acid hydrochloride. After work-up, the filtrate was concentrated under reduced pressure to afford crude (S)-4-(tert-butoxycarbonyl)morpholine-3-carboxylic acid as a solid. This material was used without further purification.

Yield: 89%; ¹H NMR (400 MHz, DMSO-d₆): δ 13.04 (br s, 1H), 4.37-4.27 (m, 1H), 4.24-4.07 (m, 1H), 3.86-3.72 (m, 1H), 3.60-3.48 (m, 2H), 3.41-3.28 (m, 1H), 3.23-2.94 (m, 1H), 1.49-1.28 (m, 9H); LCMS (Method F): Rt=1.33 min, m/z=230.0 [M−H]⁻, 98.36%.

### Step 2. tert-Butyl (S)-3-carbamoylmorpboline-4-carboxylate

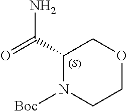

This compound was synthesized following the procedure described for the synthesis of Example 26, step 2, starting with 1 equivalent of (S)-4-(tert-butoxycarbonyl)morpholine-3-carboxylic acid. After work-up, the crude product was purified by silica gel flash column chromatography using MeOH in DCM (product eluted at 2% MeOH in DCM) to obtain tert-butyl (S)-3-carbamoylmorpholine-4-carboxylate.

Yield: 98%; LCMS-ELSD (Method D): Rt=1.28 min, m/z=131.3 [M+H−100]⁺, 77.79%.

### Step 3. tert-Butyl (R)-3-(aminomethyl)morpholine-4-carboxylate

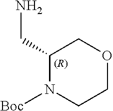

This compound was synthesized following the procedure described for the synthesis of Example 26, step 3, starting with 1 equivalent of tert-butyl (S)-3-carbamoylmorpholine-4-carboxylate. After completion, the reaction was quenched with MeOH, and the reaction was stirred at 75° C. for 30 min. The reaction was cooled to RT, and the mixture was concentrated under reduced pressure to afford crude tert-butyl (R)-3-(aminomethyl)morpholine-4-carboxylate. This material was used without further purification.

Yield: 98%.

### Step 4. tert-Butyl (R)-3-((((benzyloxycarbonylamino)methyl)morpholine-4-carboxylate

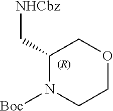

This compound was synthesized following the procedure described for the synthesis of Example 26, step 4, starting with 1 equivalent of tert-butyl (R)-3-(aminomethyl)morpholine-4-carboxylate, except that 1.7 equivalents of Cbz-Cl was used. After work-up, the crude product was purified by silica gel flash column chromatography using MeOH in DCM (product eluted at 2% MeOH in DCM) to afford tert-butyl (R)-3-((((benzyloxy)carbonyl)amino)methyl)morpholine-4-carboxylate as a gummy solid.

Yield: 20.59%; LCMS-ELSD (Method D): Rt=1.99 min, m/z=251.1 [M+H−100]¹, 77.69%.

### Step 5. Benzyl (R)-(morpholin-3-ylmethyl)carbamate hydrochloride

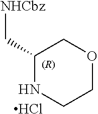

This compound was synthesized following the procedure described for the synthesis of Example 26, step 5, starting with 1 equivalent of tert-butyl (R)-3-((((benzyloxy)carbonyl)amino)methyl)morpholine-4-carboxylate, except that 5 equivalents of TMSCl was used. After completion, the reaction was concentrated under reduced pressure to afford crude benzyl (R)-(morpholin-3-ylmethyl)carbamate hydrochloride as a gum. This material was used without further purification.

Yield: 78%; LCMS-ELSD (Method D): Rt=1.40 min, m/z=251.2 [M+H]⁺, 79.10%.

### Step 6. Benzyl (R)-((4-((2-(5-(2-(ethyl(isopropyl)carbamoyl)-4-fluorophenoxy)pyrimidin-4-yl)-2-azaspiro[3.3]heptan-6-yl)methyl)morpholin-3-yl)methyl)carbamate

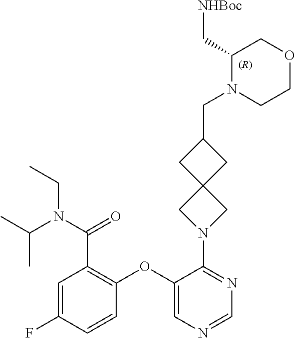

This compound was synthesized following the procedure described for the synthesis of Example 26, step 6, starting with 1 equivalent of N-ethyl-5-fluoro-2-((4-(6-formyl-2-azaspiro[3.3]heptan-2-yl)pyrimidin-5-yl)oxy)-N-isopropylbenzamide and 1.2 equivalents of benzyl (R)-(morpholin-3-ylmethyl)carbamate hydrochloride. After work-up, the crude product was purified by Prep HPLC (Method Q) to obtain benzyl (R)-((4-((2-(5-(2-(ethyl(isopropyl)carbamoyl)-4-fluorophenoxy)pyrimidin-4-yl)-2-azaspiro[3.3]heptan-6-yl)methyl)morpholin-3-yl)methyl)carbamate as a sticky solid.

Yield: 8.49%; LCMS (Method D): Rt=1.97 min, m/z=661.2 [M+H]⁺, 97.78%.

### Step 7. (R)-2-((4-(6-((3-(Aminomethylmorpholino)methyl)-2-azaspiro[3.3]heptan-2-yl)pyrimidin-5-v)oxy)-N-ethyl-5-fluoro-N-isopropylbenzamide

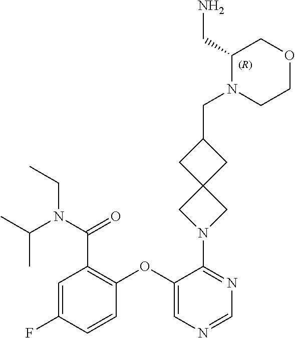

This compound was synthesized following the procedure described for the synthesis of Example 26, step 7, starting with 1 equivalent of benzyl (R)-((4-((2-(5-(2-(ethyl(isopropyl)carbamoyl)-4-fluorophenoxy)pyrimidin-4-yl)-2-azaspiro[3.3]heptan-6-yl)methyl)morpholin-3-yl)methyl)carbamate, except that 0.5 equivalent of Pd—C was used. After completion, the reaction was filtered through a Celite® pad, the pad was washed with MeOH, and the filtrate was concentrated under reduced pressure. The crude was purified by Prep HPLC (Method F) to afford (R)-2-((4-(6-((3-(aminomethyl)morpholino)methyl)-2-azaspiro[3.3]heptan-2-yl)pyrimidin-5-yl)oxy)-N-ethyl-5-fluoro-N-isopropylbenzamide as a solid.

Yield: 55.9%; ¹H NMR (400 MHz, DMSO-d₆): δ 8.31-8.22 ((m, 1H), 7.79-7.64 ((m, 1H), 7.35-7.19 (m, 2H), 7.07-6.94 (m, 1H), 4.47-3.92 (m, 4H), 3.75 (spt, J:=6.6 Hz, 1H), 3.70-3.55 (m, 2H), 3.45-3.36 (m, 3H), 3.26-3.10 (m, 2H), 2.66-2.58 (m, 2H), 2.31-2.07 (m, 6H), 1.87-1.76 (m, 2H), 1.22-0.97 (m, 9H), three protons merged with solvent peaks; LCMS (Method D): Rt=1.52 min, m/z=527.2 [M+H]⁺; HPLC (Method A): Rt=4.39 min, 99.75%; Chiral SFC (Method O): Rt=2.87 min, 100%.

### Example 28. (A)-N-Ethyl-5-fluoro-2-((4-(6-(((trans)-hexahydro-2H-pyrrolo[3,4-b][1,4]oxazepin-5(5aH)-yl)methyl)-2-azaspiro[3.3]heptan-2-yl)pyrimidin-5-yl)oxy)-N-isopropylbenzamide (Compound No. 28)

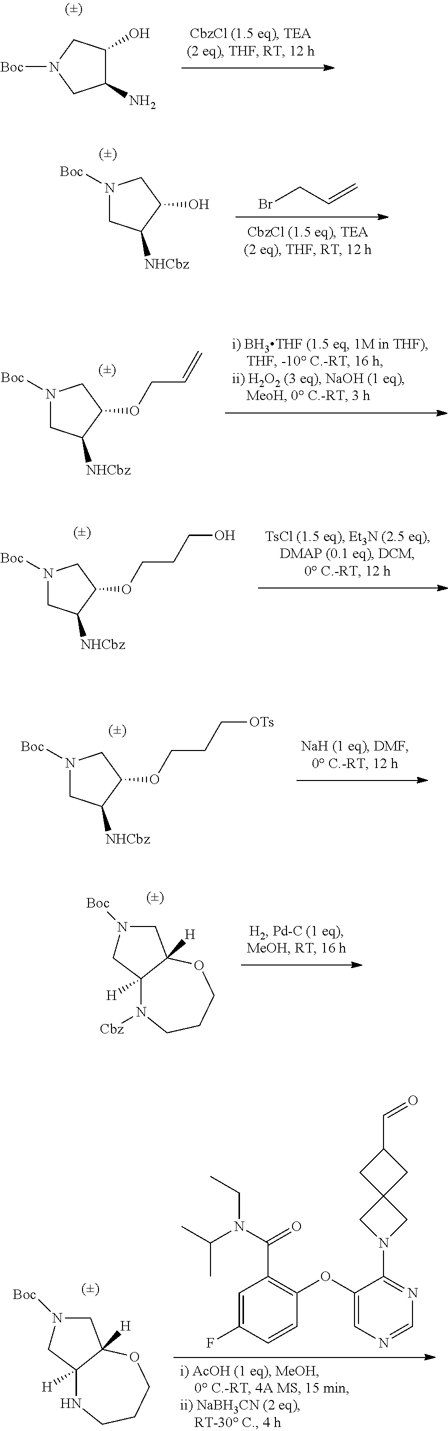

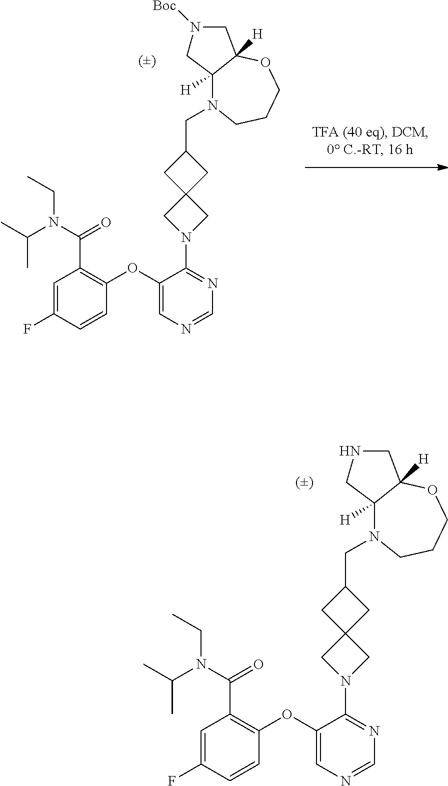 [SMILES: CCN(C(=O)c1cc(F)ccc1Oc1cncnc1N1CC2(CC(CN3CCCO[C@H]4CN(C(=O)OC(C)(C)C)C[C@@H]43)C2)C1)C(C)C ; CCN(C(=O)c1cc(F)ccc1Oc1cncnc1N1CC2(CC(CN3CCCO[C@H]4CNC[C@@H]43)C2)C1)C(C)C]

### Step 1. (±)-tert-Butyl (trans)-3-(((benzyloxycarbonyl)amino)-4-hydroxypyrrolidine-1-carboxylate

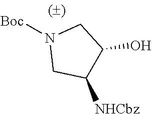 [SMILES: CC(C)(C)OC(=O)N1C[C@H](NC(=O)OCc2ccccc2)[C@@H](O)C1]

To a stirred solution of (±)-tert-butyl (trans)-3-amino-4-hydroxypyrrolidine-1-carboxylate (0.1 g, 0.494 mmol) in THF (5 mL), TEA (0.138 mL, 0.989 mmol) and Cbz-Cl (0.106 mL, 0.742 mmol) were added at RT. The reaction was stirred at RT for 12 h, and monitored by TLC (30% EtOAc in hexane) and LCMS. After completion, the reaction was quenched with saturated sodium chloride (10 mL), and the mixture was extracted with EtOAc (2×50 mL). The combined organic layer was washed with brine (50 mL), dried over anhydrous sodium sulfate and filtered, and the filtrate was concentrated under reduced pressure. The crude material was purified by silica gel flash column chromatography using EtOAc in hexane (product eluted at 80 to 100% EtOAc in hexane) to afford (±)-tert-butyl (trans)-3-(((benzyloxy)carbonyl)amino)-4-hydroxypyrrolidine-1-carboxylate (80 mg, 46.7% yield) as a liquid. ¹H NMR (400 MHz, DMSO-d₆): δ 7.61-7.48 (m, 1H), 7.41-7.28 (m, 5H), 5.29 (d, J=3.9 Hz, 1H), 5.03 (s, 2H), 4.00-3.95 (m, 1H), 3.80-3.73 (m, 1H), 3.53-3.37 (m, 2H), 3.16-3.06 (m, 2H), 1.39 (s, 9H); LCMS (Method F): Rt=1.73 min, m/z=237.2 [M+H−100]⁺, 97.80%.

### Step 2. (±)-tert-Butyl (trans)-3-(allyloxy)-4-(((benzyloxy)carbonyl)amino)pyrrolidine-1-carboxylate

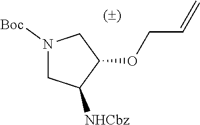 [SMILES: C=CCO[C@H]1CN(C(=O)OC(C)(C)C)C[C@@H]1NC(=O)OCc1ccccc1]

To a stirred solution of (±)-tert-butyl (trans)-3-(((benzyloxy)carbonyl)amino)-4-hydroxypyrrolidine-1-carboxylate (300 mg, 0.892 mmol) in THF (5 mL), NaH (42.8 mg, 1.070 mmol) was added at 0° C. The reaction was stirred at the same temperature for 10 min. To this reaction mixture, 3-bromoprop-1-ene (0.078 mL, 0.892 mmol) was added at 0° C. The reaction was stirred at RT for 16 h, and monitored by TLC (50% EtOAc in hexane) and LCMS. The reaction was quenched with saturated sodium chloride (10 mL), and the mixture was extracted with EtOAc (2×50 mL). The combined organic layer was washed with brine (50 mL), dried over anhydrous sodium sulfate and filtered, and the filtrate was concentrated under reduced pressure. The crude material was purified by silica gel flash column chromatography using EtOAc in hexane (product eluted at 80 to 100% EtOAc in hexane) to afford (±)-tert-butyl (trans)-3-(((benzyloxy)carbonyl)amino)-4-hydroxypyrrolidine-1-carboxylate (800 mg, 95% yield) as a liquid. LCMS (Method F): Rt=2.28 min, m/z=277.3 [M+H−100]⁺, 96.90%.

### Step 3. (±)-tert-Butyl (trans)-3-(((benzyloxy)carbonyl)amino)-4-(3-hydroxypropoxy)pyrrolidine-1-carboxylate

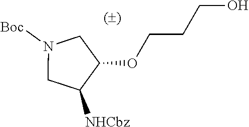 [SMILES: CC(C)(C)OC(=O)N1C[C@H](NC(=O)OCc2ccccc2)[C@@H](OCCCO)C1]

In a dried, 250 mL three neck round bottom flask under a nitrogen atmosphere, (i)-tert-butyl (trans)-3-(allyloxy)-4-(((benzyloxy)carbonyl)amino)pyrrolidine-1-carboxylate (300 mg, 0.797 mmol) was dissolved in THF (5 mL). To this solution, BH₃·THF (1.195 mL, 1.195 mmol. 1 M in THF) was added at −10° C. The reaction was warmed to RT, and the mixture was stirred for 16 h. After completion, the reaction was quenched with MeOH (15 mL) at 0° C. and the mixture was stirred for 15 min. To this mixture, NaOH (0.266 mL, 0.797 mmol) and H₂O₂ (271 mg, 2.391 mmol) were added at 0° C. The reaction was stirred at RT for 3 h, and monitored by TLC (50% EtOAc in hexane). After completion, the reaction was diluted with water (50 mL), and the mixture was extracted with EtOAc (3×50 mL). The combined organic layer was washed with brine (50 mL), dried over anhydrous sodium sulfate and filtered, and the filtrate was concentrated under reduced pressure to afford crude (±)-tert-butyl (trans)-3-(((benzyloxy)carbonyl)amino)-4-(3-hydroxypropoxy)pyrrolidine-1-carboxylate (180 mg, 57.3% yield). This material was used without further purification. LCMS (Method B): Rt=1.74 min, m/z=295.3 [M+H−100]⁺, 62.84%.

### Step 4. (±)-tert-Butyl (trans)-3-(((benzyloxy)carbonyl)amino)-4-(3-(tosyloxy)propoxy)pyrrolidine-1-carboxylate

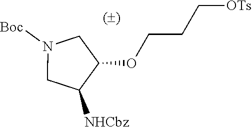 [SMILES: Cc1ccc(S(=O)(=O)OCCCO[C@H]2CN(C(=O)OC(C)(C)C)C[C@@H]2NC(=O)OCc2ccccc2)cc1]

To a stirred solution of (i)-tert-butyl (trans)-3-(((benzyloxy)carbonyl)amino)-4-(3-hydroxypropoxy)pyrrolidine-1-carboxylate (40 mg, 0.101 mmol) in DCM (2 mL), TEA (0.035 mL, 0.254 mmol), TsCl (29.0 mg, 0.152 mmol), and DMAP (1.239 mg, 10.14 μmol) were added at 0° C. The reaction was stirred at RT for 12 h. and monitored by TLC (10% MeOH in DCM). After completion, the reaction was filtered through a Celite® pad, and the pad was washed with 10% MeOH in DCM. The filtrate was washed with water (25 mL) and brine solution (25 mL). The organic layer was dried over anhydrous sodium sulfate and filtered, and the filtrate was concentrated under reduced pressure to afford crude (±)-tert-butyl (trans)-3-(((benzyloxy)carbonyl)amino)-4-(3-(tosyloxy)propoxy)pyrrolidine-1-carboxylate (30 mg, 36.7% yield). This material was used without further purification. LCMS (Method B): Rt=3.12 min, m/z=449.1 [M+H−100]⁺, 68.94%.

### Step 5. (±)-5-Benzyl 7-(tert-butyl) (trans)-hexahydro-2H-pyrrolo[3,4-b][1,4]oxazepine-5,7-dicarboxylate

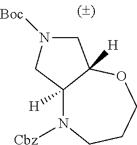 [SMILES: CC(C)(C)OC(=O)N1C[C@@H]2OCCCN(C(=O)OCc3ccccc3)[C@H]2C1]

To a stirred solution of (±)-tert-butyl (trans)-3-(((benzyloxy)carbonyl)amino)-4-(3-(tosyloxy)propoxy)pyrrolidine-1-carboxylate (100 mg, 0.182 mmol) in DMF (3 mL), NaH (7.29 mg, 0.182 mmol) was added at 0° C. The reaction was stirred at RT for 12 h, and monitored by TLC (50% EtOAc in hexane). After completion, the reaction was quenched with ice cold water (10 mL) and the mixture was extracted with EtOAc (3×25 mL). The combined organic layer was washed with brine (10 mL), dried over anhydrous sodium sulfate and filtered, and the filtrate was concentrated under reduced pressure. The crude material was purified by silica gel flash column chromatography using EtOAc in hexane (product eluted at 80 to 100% EtOAc in hexane) to afford (±)-5-benzyl 7-(tert-butyl)(trans)-hexahydro-2H-pyrrolo[3,4-b][1,4]oxazepine-5,7-dicarboxylate as (35 mg, 49.5% yield) as a liquid. ¹H NMR (400 MHz, DMSO-d₆): δ 7.46-7.26 (m, 5H), 5.29-4.92 (m, 2H), 4.42-4.09 (m, 2H), 3.98-3.74 (m, 3H), 3.65-3.39 (m, 3H), 3.04-2.88 (m, 2H), 1.93-1.79 (m, 1H), 1.66-1.51 (m, 1H), 1.44-1.31 (m, 9H); LCMS (Method B): Rt=2.89 min, m/z=277.3 [M+H−100]⁺, 97.04%.

### Step 6. (±)-tert-Butyl (trans)-octahydro-7H-pyrrolo[3,4-b][1,4]oxazepine-7-carboxylate

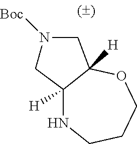 [SMILES: CC(C)(C)OC(=O)N1C[C@@H]2NCCCO[C@H]2C1]

To a stirred solution of 5-benzyl 7-tert-butyl) (trans)-hexahydro-2H-pyrrolo[3,4-b][1,4]oxazepine-5,7-dicarboxylate (35 mg, 0.093 mmol) in MeOH (10 mL), Pd—C (19.79 mg, 0.093 mmol) was added under a nitrogen atmosphere at RT. The reaction was stirred at RT for 3 h under a hydrogen atmosphere (balloon pressure), and monitored by TLC (10% MeOH in DCM). After completion, the reaction was filtered through a Celite® pad, and the pad was washed with MeOH (20 mL). The filtrate was concentrated under reduced pressure to afford crude tert-butyl (trans)-octahydro-7H-pyrrolo[3,4-b][1,4]oxazepine-7-carboxylate (20 mg, 82% yield). This material was used without further purification. ¹H NMR (400 MHz, DMSO-d₆): δ3.95-3.85 (m, 1H), 3.84-3.68 (m, 2H), 3.64-3.48 (m, 2H), 3.15-3.04 (m, 1H), 3.01-2.71 (m, 4H), 1.90-1.80 (m, 1H), 1.44-1.35 (m, 9H), 1.28-1.18 (m, 1H), one proton merged with solvent peaks; LCMS (Method B): Rt=1.44 min, m/z=243.3 [M+H]⁺, 92.16%.

### Step 7. (±)-tert-Butyl (5aS,8aS)-5-((2-(5-(2-(ethyl(isopropyl)carbamoyl)-4-fluorophenoxy)pyrimidin-4-yl)-2-azaspiro[3.3]heptan-6-yl)methyl)octahydro-7H-pyrrolo[3,4-b][1,4]oxazepine-7-carboxylate

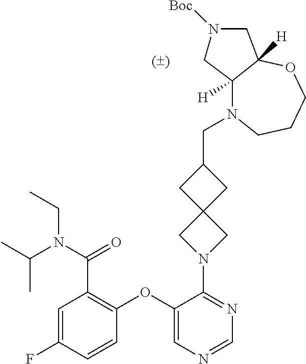 [SMILES: CCN(C(=O)c1cc(F)ccc1Oc1cncnc1N1CC2(CC(CN3CCCO[C@H]4CN(C(=O)OC(C)(C)C)C[C@@H]43)C2)C1)C(C)C]

To a stirred solution of N-ethyl-5-fluoro-2-((4-(6-formyl-2-azaspiro[3.3]heptan-2-yl)pyrimidin-5-yl)oxy)-N-isopropylbenzamide (60 mg, 0.141 mmol) and (±)-tert-butyl (trans)-octahydro-711-pyrrolo[3,4-b][1,4]oxazepine-7-carboxylate (34.1 mg, 0.141 mmol) in dry MeOH (20 mL), AcOH (8.45 mg, 0.141 mmol) and 4 Å molecular sieves were added at RT. The reaction was stirred at the same temperature for 15 min, then NaBH₃CN (17.68 mg, 0.281 mmol) was added at RT. The reaction was stirred at RT for 10 min, then at 80° C. for 4 h. The reaction progress was monitored by TLC (10% MeOH in DCM). After completion, the reaction was filtered through a Celite® pad, the pad was washed with MeOH (20 mL), and the filtrate was concentrated under reduced pressure. The crude was purified by Prep HPLC (Method F) to obtain (±)-tert-butyl (5aS,8aS)-5-((2-(5(2-(ethyl(isopropyl)carbamoyl)-4-fluorophenoxy)pyrimidin-4-yl)-2-azaspiro[3.3]heptan-6-yl)methyl)octahydro-7H-pyrrolo[3,4-b][1,4]oxazepine-7-carboxylate (25 mg, 25.7% yield) as a solid. LCMS (Method D): Rt=2.12 min, m/z=653.6 [M+H]⁺, 94.45%.

### Step 8. (±)-N-Ethyl-S-fluoro-2-((4-(6-(((trans)-hexahydro-2H-pyrrolo[3,4-b][1,4]oxazepin-5(5aH)-yl)methyl)-2-azaspiro[3.3]heptan-2-yl)pyrimidin-5-yl)oxy)-N-isopropylbenzamide

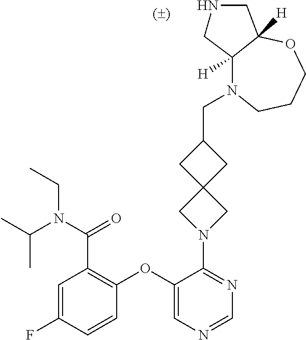 [SMILES: CCN(C(=O)c1cc(F)ccc1Oc1cncnc1N1CC2(CC(CN3CCCO[C@H]4CNC[C@@H]43)C2)C1)C(C)C]

(±)-tert-Butyl (5aS,8aS)-5-((2-(5-(2-(ethyl(isopropyl)carbamoyl)-4-fluorophenoxy)pyrimidin-4-yl)-2-azaspiro[3.3]heptan-6-yl)methyl)octahydro-7H-pyrrolo[3,4-b][1,4]oxazepine-7-carboxylate (25 mg, 0.038 mmol) was dissolved in DCM (10 mL). To this solution, TFA (0.742 mL, 9.70 mmol) was added at 0° C., and the reaction was stirred at RT for 16 h. The reaction was monitored by TLC (100% EtOAc). The reaction was concentrated under reduced pressure, the residue was dissolved in sodium bicarbonate solution (25 mL), and the mixture was extracted with 10% MeOH in DCM (3×25 mL). The combined organic layer was dried over anhydrous sodium sulfate and filtered, and the filtrate was concentrated under reduced pressure. The crude was purified by Prep HPLC (Method F) to obtain (±)-N-ethyl-5-fluoro-2-((4-(6-(((trans)-hexahydro-2H-pyrrolo[3,4-b][1,4]oxazepin-5(5aH)-yl)methyl)-2-azaspiro[3.3]heptan-2-yl)pyrimidin-5-yl)oxy)-N-isopropylbenzamide (12.7 mg, 58.3% yield) as a solid. ¹H NMR (400 MHz, DMSO-d₆): δ 8.34-8.19 (m, 1H), 7.79-7.61 (m, 1H), 7.36-7.18 (m, 2H), 7.07-6.93 (m, 1H), 4.47-3.88 (m, 5H), 3.81-3.52 (m, 4H), 3.47-3.40 (m, 1H), 3.26-3.05 (m, 3H), 3.05-2.73 (m, 3H), 2.65-2.55 (m, 1H), 2.40-2.15 (m, 4H), 1.87-1.69 (m, 4H), 1.21-0.97 (m, 9H), two protons merged with solvent peaks; LCMS (Method D): Rt=1.54 min, m/z 553.6 [M+H]⁺; HPLC (Method A): Rt=4.44 min, 98.02%; Chiral SFC (Method G): Rt=1.19 min, 99.40%.

### Example 29. 2-((4-(6-((2-Amino-1H-imidazol-1-yl)methyl)-2-azaspiro[3.3]heptan-2-yl)pyrimidin-5-yl)oxy)-N-ethyl-5-fluoro-N-isopropylbenzamide (Compound No. 29)

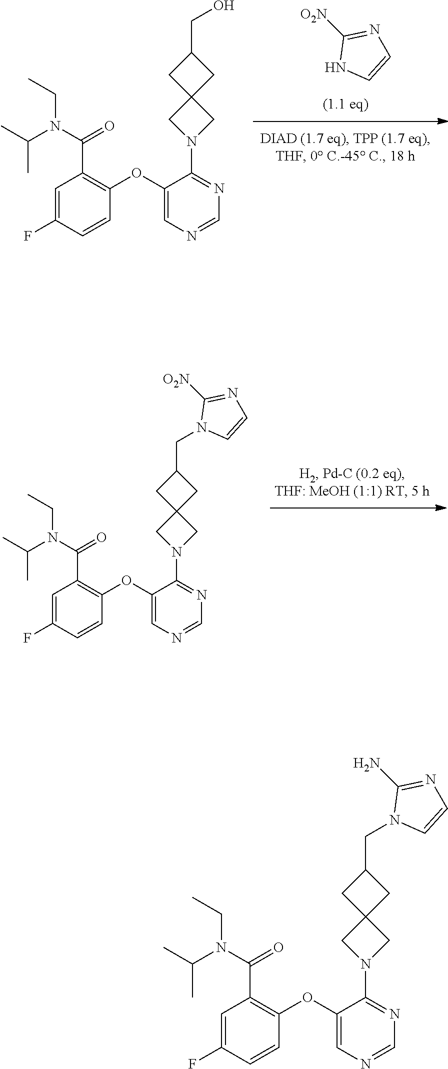 [SMILES: CCN(C(=O)c1cc(F)ccc1Oc1cncnc1N1CC2(CC(CO)C2)C1)C(C)C ; O=[N+]([O-])c1ncc[nH]1 ; CCN(C(=O)c1cc(F)ccc1Oc1cncnc1N1CC2(CC(Cn3ccnc3[N+](=O)[O-])C2)C1)C(C)C ; CCN(C(=O)c1cc(F)ccc1Oc1cncnc1N1CC2(CC(Cn3ccnc3N)C2)C1)C(C)C]

### Step 1. N-Ethyl-5-fluoro-N-isopropyl-2-((4-(6-((2-nitro-1H-imidazol-1-yl)methyl)-2-azaspiro[3.3]heptan-2-yl)pyrimidin-5-yl)oxy)benzamide

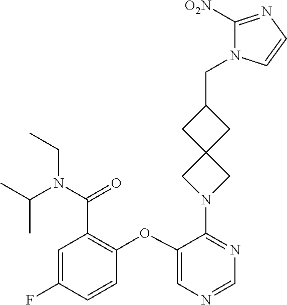 [SMILES: CCN(C(=O)c1cc(F)ccc1Oc1cncnc1N1CC2(CC(Cn3ccnc3[N+](=O)[O-])C2)C1)C(C)C]

The reaction was performed following the same procedure in three individual batches (30 mg, 150 mg, and 175 mg).

To a stirred solution of N-ethyl-5-fluoro-2-((4-(6-(hydroxymethyl)-2-azaspiro[3.3]heptan-2-yl)pyrimidin-5-yl)oxy)-N-isopropylbenzamide (175 mg, 0.408 mmol) in THE (4 mL), TPP (182 mg, 0.694 mmol) was added at RT. DIAD (140 mug, 0.694 mmol) and 2-nitro-1H-imidazole (50.8 mg, 0.449 mmol) were then added at RT. The reaction was stirred at 45° C. for 18 h, and monitored by TLC (10% MeOH in DCM). After completion, the reaction mixtures from all three batches were combined and concentrated under reduced pressure. The crude product was purified by silica-gel column chromatography using MeOH in DCM (product eluted at 5% MeOH in DCM) to obtain N-ethyl-5-fluoro-N-isopropyl-2-((4-(6-((2-nitro-1H-imidazol-1-yl)methyl)-2-azaspiro[3.3]heptan-2-yl)pyrimidin-5-yl)oxy)benzamide (195 mg, 39.3% yield) as a gummy liquid. LCMS (Method F): Rt=1.63 min, m/z=524.3 [M+H]⁺, 43.05%.

### Step 2. N-Ethyl-5-fluoro-N-isopropyl-2-((4-(6-((2-nitro-H-imidazol-1-yl)methyl)-2-azaspiro[3.3]heptan-2-yl)pyrimidin-5-yl)oxy)benzamide

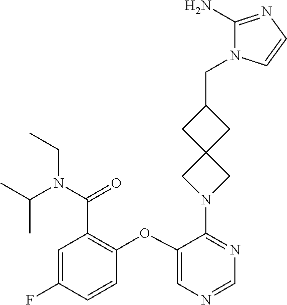 [SMILES: CCN(C(=O)c1cc(F)ccc1Oc1cncnc1N1CC2(CC(Cn3ccnc3N)C2)C1)C(C)C]

To a stirred solution of N-ethyl-5-fluoro-N-isopropyl-2-((4-(6-((2-nitro-1H-imidazol-1-yl)methyl)-2-azaspiro[3.3]heptan-2-yl)pyrimidin-5-yl)oxy)benzamide (0.3 g, 0.573 mmol) in 1:1 THF:MeOH (2 mL:2 mL), Pd—C (0.122 g, 0,115 mmol) was added at RT under a nitrogen atmosphere. The reaction was stirred at RT for 5 h under a hydrogen atmosphere (balloon pressure), and monitored by TLC (10% MeOH in DCM). After completion, the reaction was filtered through a Celite® pad, the pad was washed with 10% MeOH in DCM (20 mL), and the filtrate was concentrated under reduced pressure. The crude was purified by prep HPLC (Method A) to obtain 2-((4-(6-((2-amino-1H-imidazol-1-yl)methyl)-2-azaspiro[3.3]heptan-2-yl)pyrimidin-5-yl)oxy)-N-ethyl-5-fluoro-N-isopropylbenzamide (51 mg, 17.61% yield) as a yellow solid. ¹H NMR (400 MHz, DMSO-d₆): δ 8.33-8.22 (m, 1H), 7.81-7.64 (m, 1H), 7.34-7.20 (m, 2H), 7.06-6.95 (m, 1H), 6.53-6.50 (m, 1H), 6.32-6.31 (m, 1H), 5.21 (s, 2H), 4.23-3.97 (m, 4H), 3.75 (spt, J=6.5 Hz, 1H), 3.68-3.61 (m, 2H), 3.45-3.36 (m, 1H), 3.26-3.13 (m, 1H). 2.48-2.41 (m, 1H), 2.25-2.10 (m, 2H), 2.01-1.89 (m, 2H), 1.25-0.95 (m, 9H); LCMS (Method D): Rt=1.56 min, m/z=494.3 [M+H]⁺; HPLC (Method A): Rt=4.78 min, 97.63%.

### Example 30. 2-((4-(6-((5-Amino-1H-pyrazol-1-yl)methyl)-2-azaspiro[3.3]heptan-2-yl)pyrimidin-5-yl)oxy)-N-ethyl-5-fluoro-N-isopropylbenzamide (Compound No. 30)

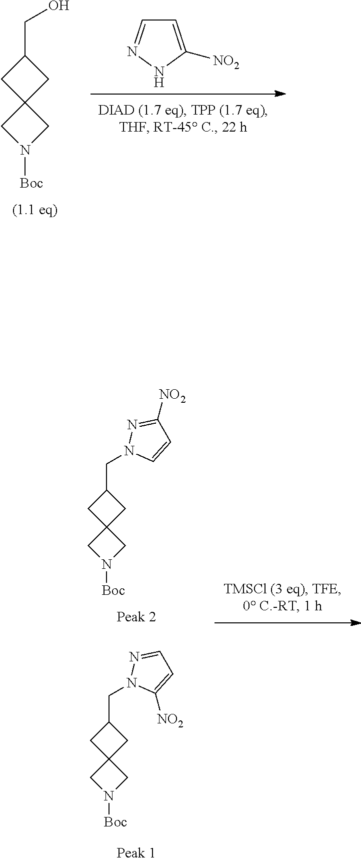 [SMILES: CC(C)(C)OC(=O)N1CC2(CC(CO)C2)C1 ; O=[N+]([O-])c1ccn[nH]1 ; CC(C)(C)OC(=O)N1CC2(CC(Cn3ccc([N+](=O)[O-])n3)C2)C1 ; CC(C)(C)OC(=O)N1CC2(CC(Cn3nccc3[N+](=O)[O-])C2)C1]

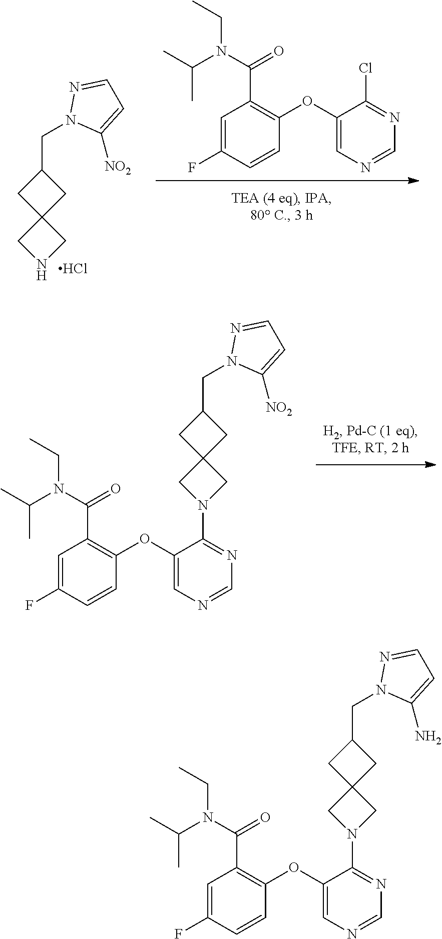 [SMILES: O=[N+]([O-])c1ccnn1CC1CC2(CNC2)C1 ; CCN(C(=O)c1cc(F)ccc1Oc1cncnc1Cl)C(C)C ; Cl ; CCN(C(=O)c1cc(F)ccc1Oc1cncnc1N1CC2(CC(Cn3nccc3[N+](=O)[O-])C2)C1)C(C)C ; CCN(C(=O)c1cc(F)ccc1Oc1cncnc1N1CC2(CC(Cn3nccc3N)C2)C1)C(C)C]

### Step 1. tert-Butyl 6-((5-nitro-1H-pyrazol-3-yl)methyl)-2-azaspiro[3.3]heptane-2-carboxylate and tert-butyl 6-((3-nitro-1H-pyrazol-1-yl)methyl)-2-azaspiro[3.3]heptane-2-carboxylate

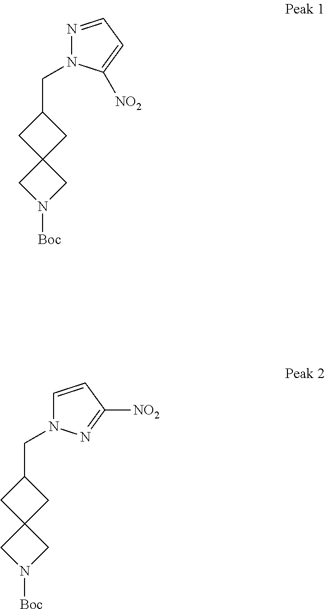 [SMILES: CC(C)(C)OC(=O)N1CC2(CC(Cn3nccc3[N+](=O)[O-])C2)C1 ; CC(C)(C)OC(=O)N1CC2(CC(Cn3ccc([N+](=O)[O-])n3)C2)C1]

This compound was synthesized following the procedure described for the synthesis of Example 29, step 1, starting with 1.1 equivalents of tert-butyl 6-(hydroxymethyl)-2-azaspiro[3.3]heptane-2-carboxylate and 1 equivalent of 5-nitro-1H-pyrazole. After completion, the reaction was concentrated under reduced pressure. The crude product was purified by silica-gel column chromatography using EtOAc in hexane (product eluted at 30%-50% EtOAc in hexane) to obtain both isomers.

Peak 1: tert-Butyl 6-((5-nitro-1H-pyrazol-1-yl)methyl)-2-azaspiro[3.3]heptane-2-carboxylate as a liquid.

Yield: 66.4%; ¹H NMR (400 MHz, DMSO-d₆): δ 7.69 (d, J=2.3 Hz, 1H), 7.27 (d, J=2.3 Hz, 1H), 4.52 (d, J=7.3 Hz, 2H), 3.92-3.66 (m, 4H), 2.74-2.58 (m, 1H), 2.24-2.12 (m, 2H), 2.04-1.91 (m, 2H), 1.36 (s, 9H); LCMS (Method F): Rt=2.08 min, m/z=223.2 [M+H−100]⁺, 94.66%.

Peak 2: tert-Butyl 6-((3-nitro-1H-pyrazol-1-yl)methyl)-2-azaspiro[3.3]heptane-2-carboxylate as a solid.

Yield: 20.41%; ¹H NMR (400 MHz, DMSO-d₆): δ 8.03 (d, J=2.5 Hz, 1H), 7.04 (d, J=2.5 Hz, 1H), 4.22 (d, J=7.4 Hz, 2H), 3.90-3.70 (m, 4H), 2.68-2.59 (m, 1H), 2.26-2.15 (m, 2H), 1.99-1.93 (m, 2H), 1.36 (s, 9H); LCMS (Method F): Rt=1.95 min, m/z=223.2 [M+H−100]⁺, 29.13%.

### Step 2. 6-((5-Nitro-11H-pyrazol-1-yl)methyl)-2-azaspiro[3.3]heptane

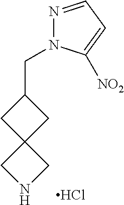 [SMILES: O=[N+]([O-])c1ccnn1CC1CC2(CNC2)C1 ; Cl]

In a 25 mL round bottom flask under a nitrogen atmosphere, tert-butyl 6-((5-nitro-1H-pyrazol-1-yl)methyl)-2-azaspiro[3.3]heptane-2-carboxylate (0.6 g, 1.861 mmol) was dissolved in TFE (10 mL). To this solution, TMSCl (0.714 mL, 5.58 mmol) was added at 0° C., and the reaction was stirred at RT for 1 h. The reaction progress was monitored by TLC (50% EtOAc in hexane). After completion, the reaction was concentrated under reduced pressure to obtain crude 6-((5-nitro-1H-pyrazol-1-yl)methyl)-2-azaspiro[3.3]heptane (0.4 g, 96% yield) as a solid. This material was used without further purification. LCMS (Method D): Rt=1.04 min, m/z=223.1 [M+H]⁺, 99.99%.

### Step 3. N-Ethyl-5-fluoro-N-isopropyl-2-((4-(6-((5-nitro-1H-pyrazol-1-yl)methyl)-2-azaspiro[3.3]heptan-2-yl)pyrimidin-5-yl)oxy)benzamide

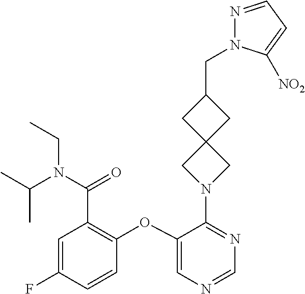 [SMILES: CCN(C(=O)c1cc(F)ccc1Oc1cncnc1N1CC2(CC(Cn3nccc3[N+](=O)[O-])C2)C1)C(C)C]

In a 25 ml round bottom flask under a nitrogen atmosphere, 6-((5-nitro-1H-pyrazol-1-yl)methyl)-2-azaspiro[3.3]heptane hydrochloride (0.4 g, 1.546 mmol) and 2-((4-chloropyrimidin-5-yl)oxy)-N-ethyl-5-fluoro-N-isopropylbenzamide (0.522 g, 1.546 mmol) were dissolved in IPA (15 mL). Et₃N (0.862 mL, 6.18 mmol) was then added at RT and the reaction was stirred at 80° C. for 3 h. The reaction progress was monitored by TLC (10% MeOH in DCM). After completion, the reaction was concentrated under reduced pressure. The residue was dissolved in EtOAc (30 mL) and the mixture was washed with water (30 mL). The organic layer was dried over anhydrous sodium sulfate and filtered, and the filtrate was concentrated under reduced pressure. The crude product was purified by silica-gel column chromatography using MeOH in DCM (product eluted at 5% MeOH in DCM) to obtain N-ethyl-5-fluoro-N-isopropyl-2-((4-(6-((5-nitro-1H-pyrazol-1-yl)methyl)-2-azaspiro[3.3]heptan-2-yl)pyrimidin-5-yl)oxy)benzamide (0.21 g, 20.79% yield) as a semi-solid. ¹H NMR (400 MHz, DMSO-d₆): δ 8.38-8.24 (m, 1H), 7.77-7.63 (m, 2H), 7.44-7.21 (m, 3H), 7.09-6.98 (m, 1H), 4.60-4.51 (m, 2H), 4.25-3.95 (m, 4H), 3.82-3.69 (m, 1H), 3.45-3.37 (m, 1H), 3.29-3.11 (m, 11H), 2.71-2.65 (m, 1H), 2.26-2.18 (m, 2H), 2.07-1.97 (m, 2H), 1.29-0.94 (m, 9H); LCMS (Method D): Rt=1.95 min, m/z=524.3 [M+H]⁺, 80.16%.

### Step 4. 2-((4-(6-((5-Amino-1H-pyrazol-1-yl)methyl)-2-azaspiro[3.3]heptan-2-yl)pyrimidin-5-yl)oxy)-N-ethyl-5-fluoro-N-isopropylbenzamide

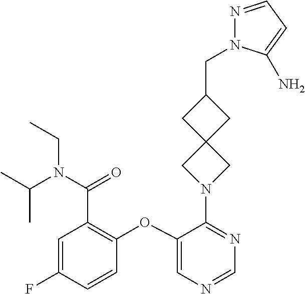 [SMILES: CCN(C(=O)c1cc(F)ccc1Oc1cncnc1N1CC2(CC(Cn3nccc3N)C2)C1)C(C)C]

This compound was synthesized following the procedure described for the synthesis of Example 29, step 2, starting with 1 equivalent of N-ethyl-5-fluoro-N-isopropyl-2-((4-(6-((5-nitro-1H-pyrazol-1-yl)methyl)-2-azaspiro[3.3]heptan-2-yl)pyrimidin-5-yl)oxy)benzamide, except that 1 equivalent of Pd—C was used. After completion, the reaction was filtered through a Celite® pad, the pad was washed with MeOH, and the filtrate was concentrated under reduced pressure. The crude was purified by Prep HPLC (Method B) to obtain 2-((4-(6-((5-amino-1H-pyrazol-1-yl)methyl)-2-azaspiro[3.3]heptan-2-yl)pyrimidin-5-yl)oxy)-N-ethyl-5-fluoro-N-isopropylbenzamide as a solid.

Yield: 21.81%; ¹H NMR (400 MHz, DMSO-d₆): δ 8.31-8.23 (m, 1H), 7.79-7.65 (m, 1H), 7.33-7.20 (m, 2H), 7.07-6.96 (m, 2H), 5.25-5.20 (m, 1H), 5.08 (s, 2H), 4.25-4.14 (m, 1H), 4.11-3.94 (m, 3H), 3.86-3.80 (m, 2H), 3.79-3.70 (m, 1H), 3.45-3.36 (m, 1H), 3.28-3.16 (m, 1H), 2.21-2.12 (m, 2H), 2.05-1.96 (m, 2H), 1.26-0.96 (m, 9H), one proton merged with solvent peaks; LCMS (Method D): Rt=1.59 min, m/z=494.3 [M+H]⁺; HPLC (Method A): Rt=4.93 min, 99.88%.

### Example 31. 2-((4-(6-((3-Amino-1H-pyrazol-1-yl)methyl)-2-azaspiro[3.3]heptan-2-Yl)pyrimidin-5-yl)oxy)-N-ethyl-5-fluoro-N-isopropylbenzamide (Compound No. 31)

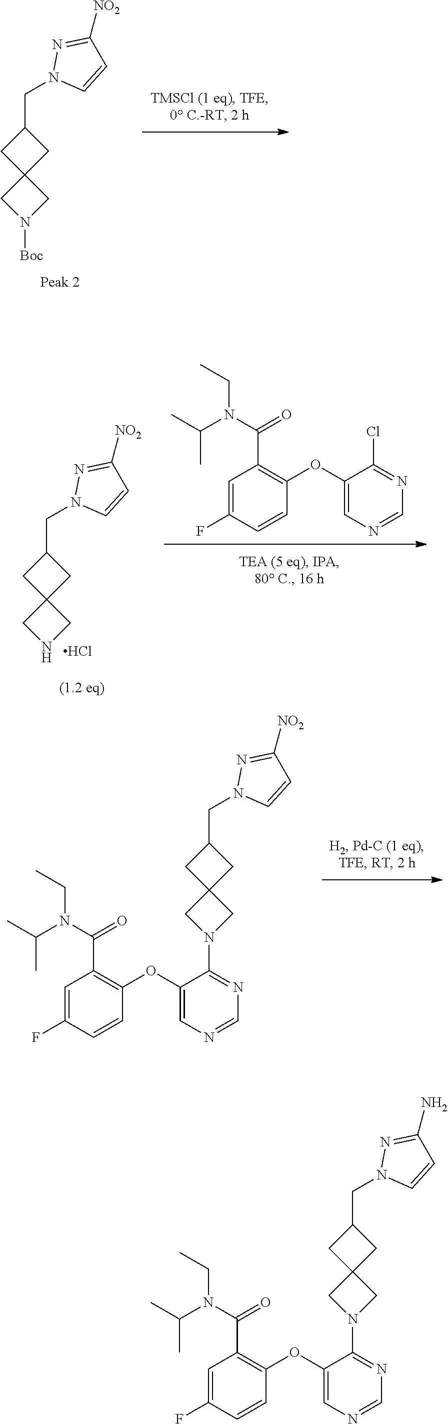 [SMILES: CC(C)(C)OC(=O)N1CC2(CC(Cn3ccc([N+](=O)[O-])n3)C2)C1 ; O=[N+]([O-])c1ccn(CC2CC3(CNC3)C2)n1 ; CCN(C(=O)c1cc(F)ccc1Oc1cncnc1Cl)C(C)C ; Cl ; CCN(C(=O)c1cc(F)ccc1Oc1cncnc1N1CC2(CC(Cn3ccc([N+](=O)[O-])n3)C2)C1)C(C)C ; CCN(C(=O)c1cc(F)ccc1Oc1cncnc1N1CC2(CC(Cn3ccc(N)n3)C2)C1)C(C)C]

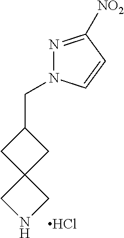 [SMILES: O=[N+]([O-])c1ccn(CC2CC3(CNC3)C2)n1 ; Cl]

This compound was synthesized following the procedure described for the synthesis of Example 30, step 2, starting with 1 equivalent of tert-butyl 6-((3-nitro-1H-pyrazol-1-yl)methyl)-2-azaspiro[3.3]heptane-2-carboxylate, except that 1 equivalent of TMSCl was used. After completion, the reaction was concentrated under reduced pressure to obtain crude 6-((3-nitro-1H-pyrazol-1-yl)methyl)-2-azaspiro[3,3]heptane hydrochloride as a solid. This material was used without further purification.

Yield: 57.1%; ¹H NMR (400 MHz, DMSO-d₆): δ 8.94 (br s, 2H), 8.04 (d, J=2.5 Hz, 1H), 7.06 (d, J=2.5 Hz, 1H), 4.23 (d, J=7.3 Hz, 2H), 3.94 (t, J=6.2 Hz, 2H), 3.84 (t, J=6.2 Hz, 2H), 2.63 (quin, J=7.9 Hz, 11H), 2.32-2.22 (m, 2H), 2.08-1.99 (m, 2H); LCMS (Method D): Rt=0.90 min, m/z=223.1 [M+H]⁺, 70.56%.

### Step 2. N-Ethyl-5-fluoro-N-isopropyl-2-((4-(6-((3-nitro-1H-pyrazol-1-yl)methyl)-2-azaspiro[3.3]heptan-2-yl)pyrimidin-5-yl)oxy)benzamide

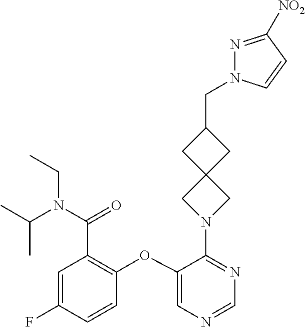 [SMILES: CCN(C(=O)c1cc(F)ccc1Oc1cncnc1N1CC2(CC(Cn3ccc([N+](=O)[O-])n3)C2)C1)C(C)C]

This compound was synthesized following the procedure described for the synthesis of Example 30, step 3, starting with 1 equivalent of 2-((4-chloropyrimidin-5-yl)oxy)-N-ethyl-S-fluoro-N-isopropylbenzamide and 1.2 equivalents of 6-((3-nitro-1H-pyrazol-1-yl)methyl)-2-azaspiro[3.3]heptane hydrochloride, except that 5 equivalents of TEA was used. After completion, the reaction was concentrated under reduced pressure. The crude product was purified by silica-gel column chromatography using MeOH in DCM (product eluted at 4% MeOH in DCM) to obtain N-ethyl-5-fluoro-N-isopropyl-2-((4-(6-((3-nitro-1H-pyrazol-1-yl)methyl)-2-azaspiro[3.3]heptan-2-yl)pyrimidin-5-yl)oxy)benzamide as a solid.

Yield: 74.7%; LCMS (Method D): Rt=1.89 min, m/z=524.1 [M+H]⁺, 87.68%.

### Step 3. 2-((4-(6-((3-Amino-1H-pyrazol-1-yl)methyl)-2-azaspiro[3.3]heptan-2-yl)pyrimidin-5-yl)oxy)-N-ethyl-S-fluoro-N-isopropylbenzamide

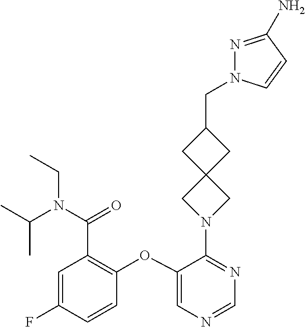 [SMILES: CCN(C(=O)c1cc(F)ccc1Oc1cncnc1N1CC2(CC(Cn3ccc(N)n3)C2)C1)C(C)C]

This compound was synthesized following the procedure described for the synthesis of Example 29, step 2, starting with 1 equivalent of N-ethyl-5-fluoro-N-isopropyl-2-((4-(6-((3-nitro-1l-pyrazol-1-yl)methyl)-2-azaspiro[3.3]heptan-2-yl)pyrimidin-5-yl)oxy)benzamide, except that 1 equivalent of Pd—C was used. After completion, the reaction was filtered through a Celite® pad, and the pad was washed with MeOH (20 mL). The filtrate was concentrated under reduced pressure. The crude was purified by reverse phase chromatography using a SiliCycle SiliaSep C18 column (40 g) on Biotage isolera one (Mobile phase A: 10 mM NH₄HCO₃ in water, B: ACN) to obtain 2-((4-(6-((3-amino-1H-pyrazol-1-yl)methyl)-2-azaspiro[3.3]heptan-2-yl)pyrimidin-5-yl)oxy)-N-ethyl-5-fluoro-N-isopropylbenzamide as a solid.

Yield: 37.3%; ¹H NMR (400 MHz, DMSO-d₆): δ8.32-8.23 (m, 1H), 7.80-7.64 (m, 1H), 7.34-7.20 (m, 3H), 7.06-6.94 (m, 11H), 5.35-5.32 (m, 11H), 4.47 (s, 2H), 4.22-4.14 (m, 1H), 4.13-3.95 (m, 3H), 3.82-3.71 (m, 3H), 3.46-3.36 (m, 1H), 3.29-3.13 (m, 1H), 2.26-2.10 (m, 2H), 2.01-1.82 (m, 2H), 1.25-0.89 (m, 9H), one proton merged with solvent peaks; LCMS (Method D): Rt=1.57 min, m/z=494.3 [M+H]⁺; HPLC (Method A): Rt=4.93 min, 99.98%.

### Example 32. N-Ethyl-5-fluoro-N-isopropyl-2-((4-(6-((4aS,8aS)-octahydro-4H-pyrido[4,3-b][1,4]oxazin-4-yl)-2-azaspiro[3.3]heptan-2-yl)pyrimidin-5-yl)oxy)benzamide (Compound No. 32)

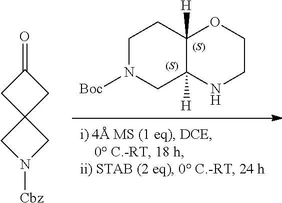

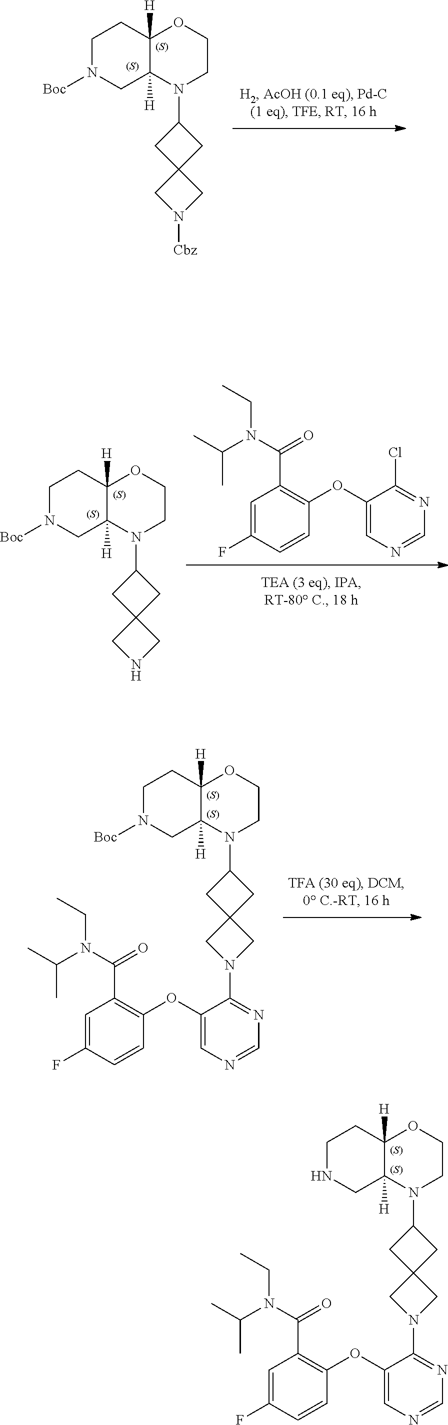

### Step 1. tert-Butyl (4aS,8aS)-4-(2-((benzyloxy)carbonyl)-2-azaspiro[3.3]heptan-6-yl)hexahydro-2H-pyrido[4,3-b][1,4]oxazine-6(5H)-carboxylate

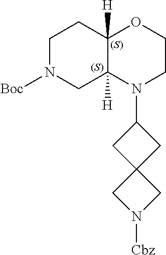

In a 50 mL, two neck round bottom flask under a nitrogen atmosphere, benzyl 6-oxo-2-azaspiro[3.3]heptane-2-carboxylate (250 mg, 0.958 mmol) was dissolved in DCE (7 mL). To this solution, tert-butyl (4aS,8aS)-hexahydro-2H-pyrido[4,3-b][1,4]oxazine-6(5H)-carboxylate (232 mg, 0.958 mmol) and 4 Å molecular sieves (250 mg, 0.958 mmol) were added at RT. The reaction was stirred at RT for 18 h, then STAB (406 mg, 1.916 mmol) was added at ° C. The reaction was stirred at RT for 24 h, and monitored by TLC (50% EtOAc in hexane). After completion, water (50 mL) was added, and the mixture was extracted with DCM (3×50 mL). The combined organic layer was dried over anhydrous sodium sulfate and filtered, and the filtrate was concentrated under reduced pressure. The crude compound was purified by silica gel flash column chromatography using EtOAc and hexane (product eluted at 75% EtOAc in hexane) to obtain tert-butyl (4aS,8aS)-4-(2-((benzyloxy)carbonyl)-2-azaspiro[3.3]heptan-6-yl)hexahydro-2H-pyrido[4,3-b][1,4]oxazine-6(5H)-carboxylate (160 mg, 33.4% yield) as a solid. ¹H NMR (400 MHz, DMSO-d₆): δ 7.43-7.27 (m, 5H), 5.01 (s, 2H), 4.29-4.10 (m, 1H), 4.04-3.73 (m, 6H), 3.55-3.47 (m, 1H), 3.31-3.30 (m, 1H), 3.17-3.10 (m, 1H), 2.83-2.69 (m, 2H), 2.66-2.60 (m, 1H), 2.27-2.20 (m, 1H), 2.15-2.06 (m, 1H), 2.03-1.94 (m, 2H), 1.75-1.66 (m, 2H), 1.40 (s, 9H), 1.33-1.23 (m, 2H); LCMS (Method B): Rt=2.15 min, m/z=472.4 [M+H]⁺, 94.35%.

### Step 2. tert-Butyl (4aS,8aS)-4-(2-azaspiro[3.3]heptan-6-yl)hexahydro-2H-pyrido[4,3-b][1,4]oxazine-6(5H)-carboxylate

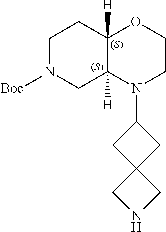

To a stirred solution of tert-butyl (4aS,8aS)-4-(2-((benzyloxy)carbonyl)-2-azaspiro[3.3]heptan-6-yl)hexahydro-2H-pyrido[4,3-b][1,4]oxazine-6(51)-carboxylate (160 mg, 0.339 mmol) in TFE (10 mL), AcOH (1.940 μL, 0.034 mmol) and Pd—C (72.2 mg, 0.339 mmol) were added under a nitrogen atmosphere at RT. The reaction was stirred at RT for 16 h under a hydrogen atmosphere (balloon pressure), and monitored by TLC (10% MeOH in DCM). After completion, the reaction was filtered through a Celite® pad, and the pad was washed with MeOH (20 mL). The filtrate was concentrated under reduced pressure to obtain crude tert-butyl (4aS,8aS)-4-(2-azaspiro[3.3]heptan-6-yl)hexahydro-2H-pyrido[4,3-b][1,4]oxazine-6(5H)-carboxylate (110 mg, 69.9% yield) as a liquid. This material was used without further purification. LCMS (Method B): Rt=0.62 min, m/z=338.3 [M+H]⁺, 72.80%.

### Step 3. tert-Butyl (4aS,8aS)-4-(2-(5-(2-(ethyl(isopropyl)carbamoyl)-4-fluorophenyl)pyrimidin-4-yl)-2-azaspiro[3.3]heptan-6-yl)hexahydro-2H-pyrido[4,3-b][1,4]oxazine-6(5H)-carboxylate

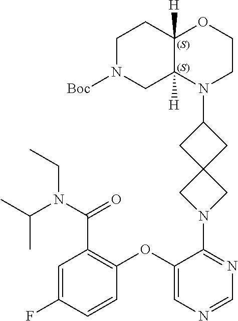

This compound was synthesized following the procedure described for the synthesis of Example 30, step 3, starting with 1 equivalent of tert-butyl (4aS,8aS)-4-(2-azaspiro[3.3]heptan-6-yl)hexahydro-2H-pyrido[4,3-b][1,4]oxazine-6(5H)-carboxylate and 1 equivalent of 2-((4-chloropyrimidin-5-yl)oxy)-N-ethyl-5-fluoro-N-isopropylbenzamide, except that 3 equivalent of TEA was used. After work-up, the crude compound was purified by silica gel flash column chromatography using EtOAc and hexane (product eluted at 75% EtOAc in hexane) to obtain tert-butyl (4aS,8aS)-4-(2-(5-(2-(ethyl(isopropyl)carbamoyl)-4-fluorophenoxy)pyrimidin-4-yl)-2-azaspiro[3.3]heptan-6-yl)hexahydro-2H-pyrido[4,3-b][1,4]oxazine-6(5H)-carboxylate as a liquid.

Yield: 70.8%; LCMS (Method D): Rt=1.87 min, m/z=639.8 [M+H]⁺, 92.19%.

### Step 4. N-Ethyl-S-fluoro-N-isopropyl-2-((4-(6-((4aS,8aS)-octahydro-4H-pyrido[4,3-b][1,4]oxazin-4-yl)-2-azaspiro[3.3]heptan-2-yl)pyrimidin-5-yl)oxy)benzamide

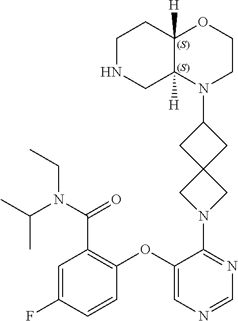

To a stirred solution of tert-butyl (4aS,8aS)-4-(2-(5-(2-(ethyl(isopropyl)carbamoyl)-4-fluorophenoxy)pyrimidin-4-yl)-2-azaspiro[3.3]heptan-6-yl)hexahydro-2H-pyrido[4,3-b][1,4]oxazine-6(5H)-carboxylate (150 mg, 0.235 mmol) in DCM (5 mL), TFA (0.54 mL, 7.04 mmol) was added at 0° C. under a nitrogen atmosphere. The reaction was stirred at RT for 16 h, and monitored by TLC (5% MeOH in DCM). After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain N-ethyl-5-fluoro-N-isopropyl-2-((4-(6-((4aS,8aS)-octahydro-4H-pyrido[4,3-b][1,4]oxazin-4-yl)-2-azaspiro[3.3]heptan-2-yl)pyrimidin-5-yl)oxy)benzamide (54.1 mg, 42.2% yield) as a solid. ¹H NMR (400 MHz, DMSO-d₆): δ 8.31-8.22 (m, 1H), 7.79-7.64 (m, 1H), 7.33-7.20 (m, 2H), 7.06-6.95 (m, 1H), 4.26-4.02 (m, 3H), 3.98-3.93 (m, 1H), 3.80-3.69 (m, 2H), 3.56-3.48 (m, 1H), 3.44-3.39 (m, 1H), 3.24-3.15 (m, 1H), 3.08-2.96 (m, 2H), 2.90-2.82 (m, 1H), 2.79-2.71 (m, 1H), 2.65-2.60 (m, 1H), 2.44-2.38 (m, 1H), 2.31-2.27 (m, 1H), 2.25-2.16 (m, 1H), 2.14-1.90 (m, 4H), 1.82-1.73 (m, 1H), 1.68-1.58 (m, 1H), 1.35-0.96 (m, 9H), two protons merged with solvent peaks; LCMS (Method D): Rt=1.39 min, m/z=539.3 [M+H]⁺; HPLC (Method A); Rt=4.16 min, 98.59%; Chiral SFC (Method G): Rt=1.22 min, 99.05%.

### Example 33. N-Ethyl-5-fluoro-N-isopropyl-2-((4-(6-((4aR,8aR)-octahydro-4H-pyrido[4,3-b][1,4]oxazin-4-yl)-2-azaspiro[3.3]heptan-2-yl)pyrimidin-5-yl)oxy)benzamide (Compound No. 33)

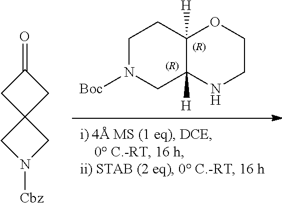

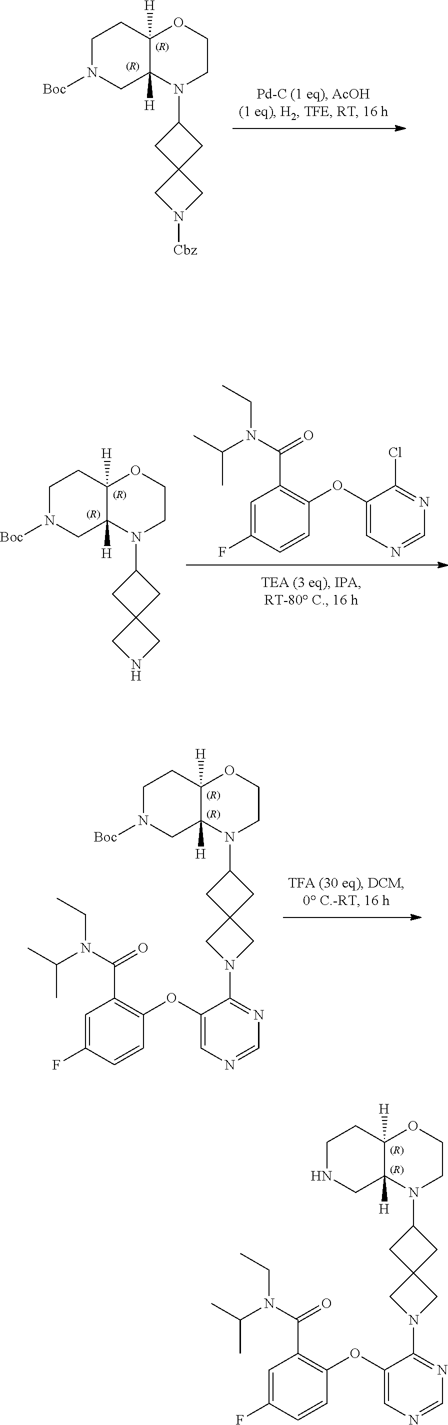

### Step 1. tert-Butyl (4aR,8aR)-4-(2-(benzyloxy)carbonyl)-2-azaspiro[3.3]heptan-6-yl)-hexahydro-2H-pyrido[4,3-b][1,4]oxazine-6(5H)-carboxylate

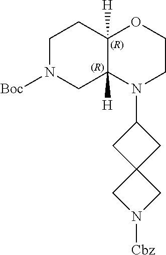

This compound was synthesized following the procedure described for the synthesis of Example 32, step 1, starting with 1 equivalent of 6-oxo-2-azaspiro[3.3]heptane-2-carboxylate and 1 equivalent of tert-butyl (4aR,8aR)-hexahydro-21-pyrido[4,3-b][1,4]oxazine-6(5H)-carboxylate. After work-up, the crude was purified by silica gel flash column chromatography using EtOAc and hexane (product eluted at 100% EtOAc) to obtain tert-butyl (4aR,8aR)-4-(2-((benzyloxy)carbonyl)-2-azaspiro[3.3]heptan-6-yl)hexahydro-2H-pyrido[4,3-b][1,4]oxazine-6(5H)-carboxylate as a sticky liquid.

Yield: 41.6%; ¹H NMR (400 MHz, DMSO-d₆): δ 7.54-7.18 (m, 5H), 5.14-4.93 (m, 2H), 4.23-4.07 (m, 2H), 4.04-3.70 (m, 5H), 3.57-3.49 ((m, 11H), 3.15-3.08 (m, 1H), 3.14-3.08 (m, 1H), 2.85-2.59 (m, 2H), 2.44-2.18 (m, 2H), 2.14-1.94 (m, 2H), 1.86-1.63 (m, 2H), 1.40 (s, 9H), 1.32-1.21 (m, 2H); LCMS (Method B): Rt=2.00 min, m/z=472.4 [M+H]⁺, 94.03%.

### Step 2. tert-Butyl (4aR,8aR)-4-(2-azaspiro[3.3]heptan-6-yl)hexahydro-2H-pyrido[4,3-b][1,4]oxazine-6(5H)-carboxylate

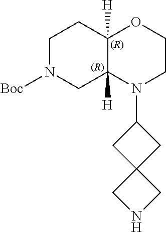

This compound was synthesized following the procedure described for the synthesis of Example 32, step 2, starting with 1 equivalent of tert-butyl (4aS,8aS)-4-(2-((benzyloxy)carbonyl)-2-azaspiro[3.3]heptan-6-yl)hexahydro-2H-pyrido[4,3-b][1,4]oxazine-6(5H)-carboxylate, except that 1 equivalent of AcOH was used. After filtration, the filtrate was concentrated under reduced pressure to obtain crude tert-butyl (4aS,8aS)-4-(2-azaspiro[3.3]heptan-6-yl)hexahydro-2H-pyrido[4,3-b][1,4]oxazine-6(5H)-carboxylate as a sticky liquid. This material was used without further purification.

Yield: 88%; LCMS (Method B): Rt=0.35 min, m/z=338.3 [M+H]⁺, 88.89%.

### Step 3. tert-Butyl (4aR,8aR)-4-(2-(5-(2-(ethyl(isopropyl)carbamoyl)-4-fluorophenoxy)pyrimidin-4-yl)-2-azaspiro[3.3]heptan-6-yl)hexahydro-2H-pyrido[4,3-b][1,4]oxazine-6(5H)-carboxylate

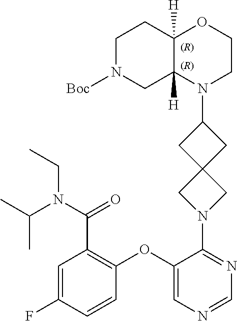

This compound was synthesized following the procedure described for the synthesis of Example 30, step 3, starting with 1 equivalent of tert-butyl (4aR,8aR)-4-(2-azaspiro[3,3]heptan-6-yl)hexahydro-2H-pyrido[4,3-b][1,4]oxazine-6(5H)-carboxylate and 1 equivalent of 2-((4-chloropyrimidin-5-yl)oxy)-N-ethyl-5-fluoro-N-isopropylbenzamide, except that 3 equivalents of TEA was used. After work-up, the crude was purified by silica gel flash column chromatography using MeOH and DCM (product eluted at 10% MeOH in DCM) to obtain tert-butyl (4aR,8aR)-4-(2-(5-(2-(ethyl(isopropyl)carbamoyl)-4-fluorophenoxy)pyrimidin-4-yl)-2-azaspiro[3.3]heptan-6-yl)hexahydro-2H-pyrido[4,3-b][1,4]oxazine-6(5H)-carboxylate as a sticky liquid.

Yield: 61.3%; LCMS (Method D): Rt=1.88 min, m/z=639.4 [M+H]⁺, 89.55%.

### Step 4. N-Ethyl-5-fluoro-N-isopropyl-2-((4-(6-((4aR,8aR)-octahydro-4H-pyrido[4,3-][1,4]oxazin-4-yl)-2-azaspiro[3.3]heptan-2-yl)pyrimidin-5-yl)oxy)benzamide

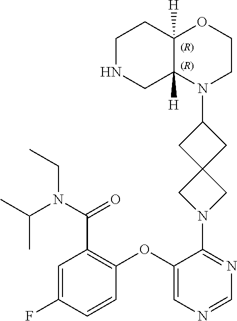

This compound was synthesized following the procedure described for the synthesis of Example 32, step 4, starting with 1 equivalent of tert-butyl (4aR,8aR)-4-(2-(5-(2-(ethyl(isopropyl)carbamoyl)-4-fluorophenoxy)pyrimidin-4-yl)-2-azaspiro[3.3]heptan-6-yl)hexahydro-2H-pyrido[4,3-b][1,4]oxazine-6(5H)-carboxylate. After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain N-ethyl-5-fluoro-N-isopropyl-2-((4-(6-((4aR,8aR)-octahydro-4H-pyrido[4,3-b][1,4]oxazin-4-yl)-2-azaspiro[3.3]heptan-2-yl)pyrimidin-5-yl)oxy)benzamide as a solid.

Yield: 29.7%; ¹H NMR (400 MHz, DMSO-d₆): δ8.30-8.24 (m, 1H), 7.78-7.64 (m, 1H), 7.37-7.19 (m, 2H), 7.12-6.95 (m, 1H), 4.31-3.92 ((m, 4H), 3.85-3.71 (m, 2H), 3.58-3.46 (m, 1H), 3.44-3.37 (m, 1H), 3.25-3.16 (m, 1H), 3.08-2.98 (m, 2H), 2.89-2.70 (m, 2H), 2.64-2.60 (m, 1H), 2.44-2.38 (m, 1H), 2.32-2.26 (m, 1H), 2.25-2.16 (m, 1H), 2.14-1.88 (m, 4H), 1.82-1.72 (m, 1H), 1.68-1.58 (m, 1H), 1.36-0.97 (m, 9H), one proton merged with solvent peaks; LCMS (Method B): Rt=1.37 min, m/z=539.4 [M+H]⁺; HPLC (Method A): Rt=4.23 min, 97.62%; Chiral SFC (Method 1): Rt=1.25 min, 95.47%.

### Example 34. (±)-N-Ethyl-5-fluoro-N-isopropyl-2-((4-(6-((trans)-octahydro-1H-pyrido[3,4-b][1,4]oxazin-1-yl)-2-azaspiro[3.3]heptan-2-yl)pyrimidin-5-yl)oxy)benzamide (Compound No. 34)

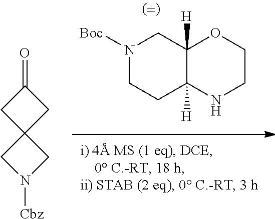 [SMILES: O=C1CC2(C1)CN(C(=O)OCc1ccccc1)C2 ; CC(C)(C)OC(=O)N1CC[C@@H]2NCCO[C@H]2C1]

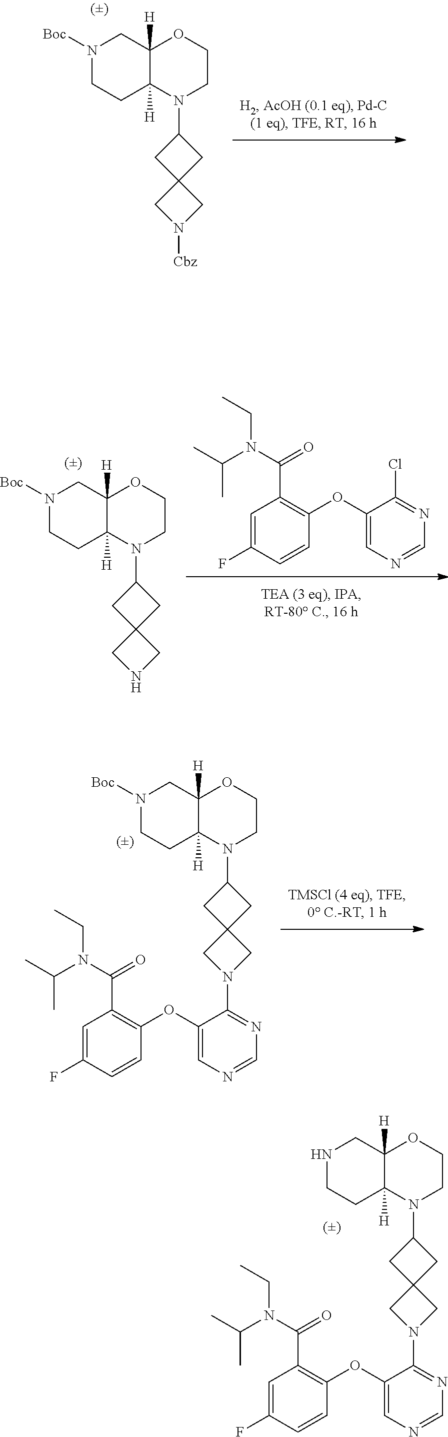 [SMILES: CC(C)(C)OC(=O)N1CC[C@H]2[C@H](C1)OCCN2C1CC2(C1)CN(C(=O)OCc1ccccc1)C2 ; CC(C)(C)OC(=O)N1CC[C@H]2[C@H](C1)OCCN2C1CC2(CNC2)C1 ; CCN(C(=O)c1cc(F)ccc1Oc1cncnc1Cl)C(C)C ; CCN(C(=O)c1cc(F)ccc1Oc1cncnc1N1CC2(CC(N3CCO[C@H]4CN(C(=O)OC(C)(C)C)CC[C@@H]43)C2)C1)C(C)C ; CCN(C(=O)c1cc(F)ccc1Oc1cncnc1N1CC2(CC(N3CCO[C@H]4CNCC[C@@H]43)C2)C1)C(C)C]

### Step 1. (±)-tert-Butyl (4aS,8aS)-1-(2-((benzyloxy)carbonyl)-2-azaspiro[3.3]heptan-6-yl)octahydro-6H-pyrido[3,4-b][1,4]oxazine-6-carboxylate

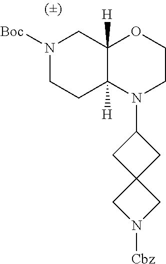 [SMILES: CC(C)(C)OC(=O)N1CC[C@H]2[C@H](C1)OCCN2C1CC2(C1)CN(C(=O)OCc1ccccc1)C2]

This compound was synthesized following the procedure described for the synthesis of Example 32, step 1, starting with 1 equivalent of benzyl 6-oxo-2-azaspiro[3.3]heptane-2-carboxylate and 1 equivalent of (i)-tert-butyl (trans)-octahydro-6H-pyrido[3,4-b][1,4]oxazine-6-carboxylate. After work-up, the crude was purified by silica gel flash column chromatography using EtOAc in hexane (product eluted at 50% EtOAc in hexane) to obtain (i)-tert-butyl (4aS,8aS)-1-(2-((benzyloxy)carbonyl)-2-azaspiro[3.3]heptan-6-yl)octahydro-6H-pyrido[3,4-b][1,4]oxazine-6-carboxylate as a sticky solid.

Yield: 37.8%; LCMS (Method B): Rt=2.16 min, m/z=472.4 [M+H]⁺, 86.43%.

### Step 2. (k)-tert-Butyl (4aS,8aS)-1-(2-azaspiro[3.3]heptan-6-yl)octahydro-6H-pyrido[3,4-b][1,4]oxazine-6-carboxylate

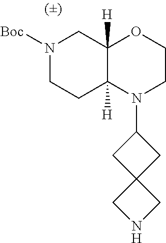 [SMILES: CC(C)(C)OC(=O)N1CC[C@H]2[C@H](C1)OCCN2C1CC2(CNC2)C1]

This compound was synthesized following the procedure described for the synthesis of Example 32, step 2, starting with 1 equivalent of (±)-tert-butyl (4aS,8aS)-1-(2-((benzyloxy)carbonyl)-2-azaspiro[3.3]heptan-6-yl)octahydro-6H-pyrido[3,4-b][1,4]oxazine-6-carboxylate. After filtration, the filtrate was concentrated under reduced pressure to obtain crude (±)-tert-butyl (4aS,8aS)-1-(2-azaspiro[3.3]heptan-6-yl)octahydro-6H-pyrido[3,4-b][1,4]oxazine-6-carboxylate as a sticky liquid. This material was used without further purification.

Yield: 79%; LCMS-ELSD (Method D): Rt=1.33 min, m/z=338.2 [M+H]⁺, 79.57%.

### Step 3. (±)-tert-Butyl (4aS,8aS)-1-(2-(5-(2-(ethyl(isopropyl)carbamoyl)-4-fluorophenoxy)pyrimidin-4-yl)-2-azaspiro[3.3]heptan-6-yl)octahydro-6H-pyrido[3,4-b][1,4]oxazine-6-carboxylate

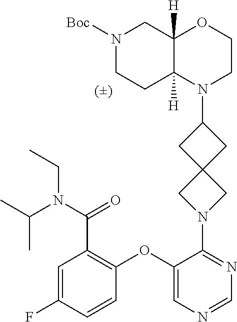 [SMILES: CCN(C(=O)c1cc(F)ccc1Oc1cncnc1N1CC2(CC(N3CCO[C@H]4CN(C(=O)OC(C)(C)C)CC[C@@H]43)C2)C1)C(C)C]

This compound was synthesized following the procedure described for the synthesis of Example 30, step 3, starting with 1 equivalent of (±)-tert-butyl (4aS,8aS)-1-(2-azaspiro[3.3]heptan-6-yl)octahydro-6H-pyrido[3,4-b][1,4]oxazine-6-carboxylate and 1 equivalent of 2-((4-chloropyrimidin-5-yl)oxy)-N-ethyl-5-fluoro-N-isopropylbenzamide, except that 3 equivalents of TEA was used. After work-up, the crude was purified by silica gel flash column chromatography using MeOH and DCM (product eluted at 5% MeOH in DCM) to obtain (±)-tert-butyl (4aS,8aS)-1-(2-(5-(2-(ethyl(isopropyl)carbamoyl)-4-fluorophenoxy)pyrimidin-4-yl)-2-azaspiro[3.3]heptan-6-yl)octahydro-6H-pyrido[3,4-b][1,4]oxazine-6-carboxylate as a sticky solid.

Yield: 53.9%; ¹H NMR (400 MHz, DMSO-d₆): δ 8.37-8.24 (m, 1H), 7.78-7.63 (m, 1H), 7.34-7.18 (m, 2H), 7.07-6.92 (m, 1H), 4.24-3.86 (m, 6H), 3.82-3.72 (m, 1H), 3.61-3.50 (m, 1H), 3.46-3.37 (m, 1H), 3.28-3.20 (m, 1H), 3.19-3.08 (m, 3H), 3.00-2.77 (m, 1H), 2.66-2.58 (m, 1H), 2.46-2.37 (m, 1H), 2.27-2.08 (m, 1H), 2.03-1.94 (m, 2H), 1.89-1.82 (m, 1H), 1.39 (s, 9H), 1.24-0.96 (m, 13H); LCMS (Method D): Rt=1.92 min, m/z=639.5 [M+H]⁺. 58.25%.

### Step 4. (±)-N-Ethyl-5-fluoro-N-isopropyl-2-((4-(6-((trans)-octahydro-1H-pyrido[3,4-b][1.4]oxazin-1-yl)-2-azaspiro[3.3]heptan-2-yl)pyrimidin-5-yl)oxy)benzamide

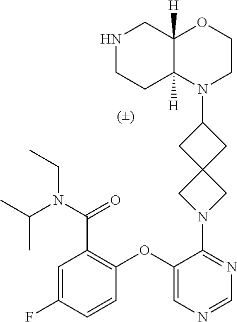 [SMILES: CCN(C(=O)c1cc(F)ccc1Oc1cncnc1N1CC2(CC(N3CCO[C@H]4CNCC[C@@H]43)C2)C1)C(C)C]

To a stirred solution of (±)-tert-butyl (4aS,8aS)-1-(2-(5-(2-(ethyl(isopropyl)carbamoyl)-4-fluorophenoxy)pyrimidin-4-yl)-2-azaspiro[3.3]heptan-6-yl)octahydro-6H-pyrido[3,4-b][1,4]oxazine-6-carboxylate (350 mg, 0.548 mmol) in TFE (10 mL), TMSCl (0.280 mL, 2.192 mmol) was added at 0° C. under a nitrogen atmosphere. The reaction was stirred at RT for 1 h, and monitored by TLC (80% EtOAc in hexane). After completion, the reaction was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain (±)-N-ethyl-5-fluoro-N-isopropyl-2-((4-(6-((trans)-octahydro-1H-pyrido[3,4-b][1.4]oxazin-1-yl)-2-azaspiro[3.3]heptan-2-yl)pyrimidin-5-yl)oxy)benzamide (51.62 mg, 17.43% yield) as a solid. ¹H NMR (400 MHz, DMSO-d₆): δ 8.31-8.23 (m, 1H), 7.80-7.65 (m, 1H), 7.35-7.21 (m, 2H), 7.06-6.94 (m, 1H), 4.25-3.92 (m, 4H), 3.80-3.72 (m, 1H), 3.59-3.47 (m, 2H), 3.25-3.19 (m, 1H), 3.05-2.95 (m, 1H), 2.87-2.74 (m, 3H), 2.65-2.59 (m, 1H), 2.40-2.29 (m, 3H), 2.27-2.19 (m, 2H), 2.15-2.07 (m, 1H), 2.03-1.91 (m, 2H), 1.81-1.71 (m, 2H), 1.28-0.94 (m, 9H), two protons merged with solvent peaks; LCMS (Method D): Rt 1.42 min, m/z=539.3 [M+H]⁺; HPLC (Method A): Rt=4.39 min, 99.65%; Chiral SFC (Method K): Peak-1: Rt=3.37 min, 49.30%, Peak-2: 3.70 min, 50.69%.

### Example 35. (±)-N-Ethyl-5-fluoro-2-((4-(6-((trans)-hexahydropyrrolo[3,4-b][1,4]oxazin-4(4aH)-yl)-2-azaspiro[3.3]heptan-2-yl)pyrimidin-5-yl)oxy)-N-isopropylbenzamide (Compound No. 35)

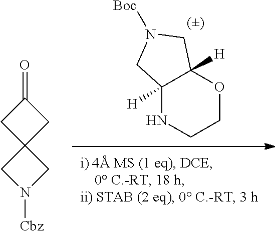 [SMILES: O=C1CC2(C1)CN(C(=O)OCc1ccccc1)C2 ; CC(C)(C)OC(=O)N1C[C@@H]2NCCO[C@H]2C1]

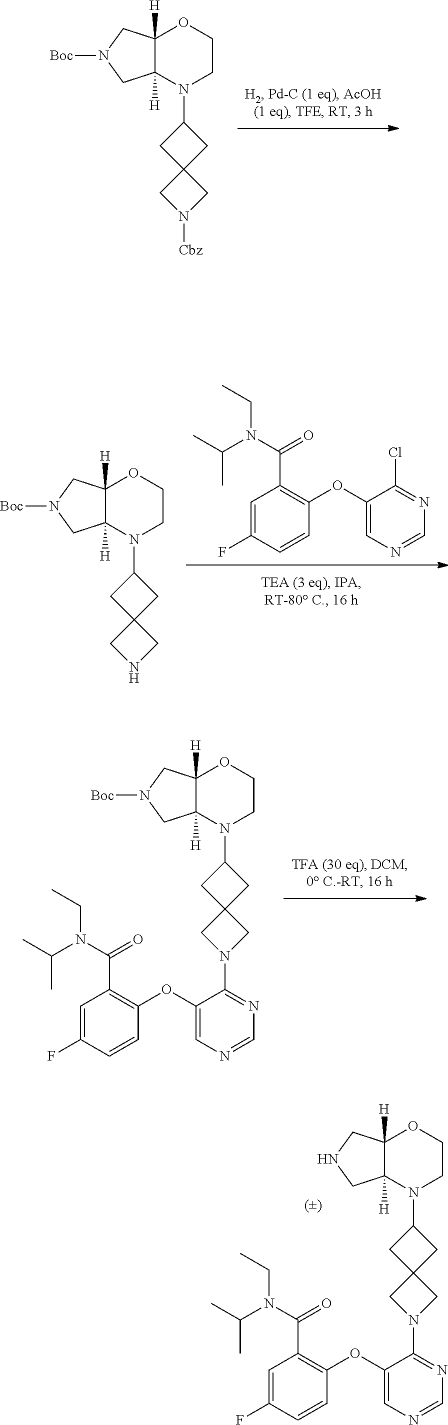 [SMILES: CC(C)(C)OC(=O)N1C[C@@H]2OCCN(C3CC4(C3)CN(C(=O)OCc3ccccc3)C4)[C@H]2C1 ; CC(C)(C)OC(=O)N1C[C@@H]2OCCN(C3CC4(CNC4)C3)[C@H]2C1 ; CCN(C(=O)c1cc(F)ccc1Oc1cncnc1Cl)C(C)C ; CCN(C(=O)c1cc(F)ccc1Oc1cncnc1N1CC2(CC(N3CCO[C@H]4CN(C(=O)OC(C)(C)C)C[C@@H]43)C2)C1)C(C)C ; CCN(C(=O)c1cc(F)ccc1Oc1cncnc1N1CC2(CC(N3CCO[C@H]4CNC[C@@H]43)C2)C1)C(C)C]

### Step 1. (±)-tert-Butyl (4aS,7aS)-4-(2-((benzyloxy)carbonyl)-2-azaspiro[3.3]heptan-6-yl)hexahydropyrrolo[3,4-b][1,4]oxazine-6(2H)-carboxylate

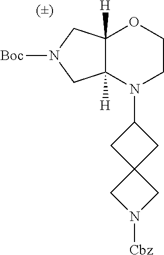 [SMILES: CC(C)(C)OC(=O)N1C[C@@H]2OCCN(C3CC4(C3)CN(C(=O)OCc3ccccc3)C4)[C@H]2C1]

This compound was synthesized following the procedure described for the synthesis of Example 32, step 1, starting with 1 equivalent of benzyl 6-oxo-2-azaspiro[3.3]heptane-2-carboxylate and 1 equivalent of (±)-tert-butyl (4aS,7aS)-hexahydropyrrolo[3,4-b][1,4]oxazine-6(2H)-carboxylate. After completion, the reaction was filtered through a Celite® pad, and the pad was washed with 10% MeOH in DCM. The filtrate was washed with water, dried over anhydrous sodium sulfate and filtered, and the filtrate was concentrated under reduced pressure. The crude was purified by silica gel flash column chromatography using EtOAc and hexane to obtain (i)-tert-butyl (4aS,7aS)-4-(2-((benzyloxy)carbonyl)-2-azaspiro[3.3]heptan-6-yl)hexahydropyrrolo[3,4-b][1.4]oxazine-6(2H)-carboxylate as a gummy solid. Yield: 44.6%; ¹H NMR (400 MHz, DMSO-d₆): δ 7.41-7.30 (m, 5H), 5.01 (s, 2H), 4.00-3.78 (m, 5H), 3.60-3.42 (m, 5H), 2.96-2.84 (m, 2H), 2.66-2.61 (m, 2H), 2.29-2.20 (m, 2H), 1.97-1.90 (m, 2H), 1.39 (s, 9H), one proton merged with solvent peaks; LCMS-ELSD (Method D): Rt=2.07 min, m/z=458.2 [M+H]⁺, 99.82%.

### S Step 2. (±)-tert-Butyl (4aS,7aS)-4-(2-azaspiro[3.3]heptan-6-yl)hexahydropyrrolo[3,4-b][1,4]oxazine-6(2H)-carboxylate

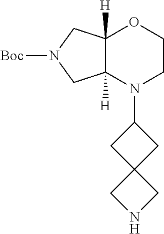 [SMILES: CC(C)(C)OC(=O)N1C[C@@H]2OCCN(C3CC4(CNC4)C3)[C@H]2C1]

This compound was synthesized following the procedure described for the synthesis of Example 32, step 2, starting with 1 equivalent of (±)-tert-butyl (4aS,7aS)-4-(2-((benzyloxy)carbonyl)-2-azaspiro[3.3]heptan-6-yl)hexahydropyrrolo[3,4-b][1,4]oxazine-6(2H)-carboxylate, except that 1 equivalent of AcOH was used. After filtration, the filtrate was concentrated under reduced pressure to obtain crude (±)-tert-butyl (4aS,7aS)-4-(2-azaspiro[3.3]heptan-6-yl)hexahydropyrrolo[3,4-b][1,4]oxazine-6(2H)-carboxylate as a gummy liquid. This material was used without further purification. Quantitative yield; LCMS-ELSD (Method B): Rt=1.29 min, m/z=324.4 [M+H], 83.75%.

### Step 3. (±)-tert-Butyl (4aS,7aS)-4-(2-(5-(2-(ethyl(isopropyl)carbamoyl)-4-fluorophenoxy)(pyrimidin-4-yl)-2-azaspiro[3.3]heptan-6-yl)hexahydropyrrolo[3,4-b][1,4]oxazine-6(2H)-carboxylate

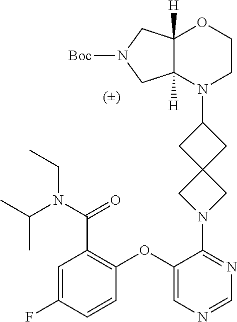 [SMILES: CCN(C(=O)c1cc(F)ccc1Oc1cncnc1N1CC2(CC(N3CCO[C@H]4CN(C(=O)OC(C)(C)C)C[C@@H]43)C2)C1)C(C)C]

This compound was synthesized following the procedure described for the synthesis of Example 30, step 3, starting with 1 equivalent of (±)-tert-butyl (4aS,7aS)-4-(2-azaspiro[3.3]heptan-6-yl)hexahydropyrrolo[3,4-b][1,4]oxazine-6(2H)-carboxylate and 1 equivalent of 2-((4-chloropyrimidin-5-yl)oxy)-N-ethyl-5-fluoro-N-isopropylbenzamide, except that 3 equivalents of TEA was used. After work-up, the crude was purified by silica gel flash column chromatography using EtOAc and hexane (product eluted at 100% EtOAc) to obtain (4)-tert-butyl (4aS,7aS)-4-(2-(5-(2-(ethyl(isopropyl)carbamoyl)-4-fluorophenoxy)pyrimidin-4-yl)-2-azaspiro[3.3]heptan-6-yl)hexahydropyrrolo[3,4-b][1,4]oxazine-6(2H)-carboxylate as a liquid.

Yield: 43.7%; ¹H NMR (400 MHz, DMSO-d₆): δ 8.37-8.22 ((m, 1H), 7.78-7.63 (m, 1H), 7.34-7.21 (m, 2H), 7.10-6.94 (m, 1H), 4.27-3.94 (m, 5H), 3.92-3.85 (m, 1H), 3.81-3.70 (m, 1H), 3.63-3.39 (m, 5H), 3.26-3.20 (m, 1H), 3.18-3.12 (m, 1H), 2.98-2.82 (m, 2H), 2.72-2.61 (m, 2H), 2.29-2.23 (m, 2H), 2.04-1.94 (m, 2H), 1.39 (s, 9H), 1.28-0.97 (m, 9H); LCMS-ELSD (Method B): Rt=1.84 min, m/z=625.4 [M+H]⁺, 92.48%.

### Step 4. (±)-N-Ethyl-5-fluoro-2-((4-(6-((trans)-hexahydropyrrolo[3,4-b][1,4]oxazin-4(4aH)-yl)-2-azaspiro[3.3]heptan-2-yl)pyrimidin-5-yl)oxy)-N-isopropylbenzamide

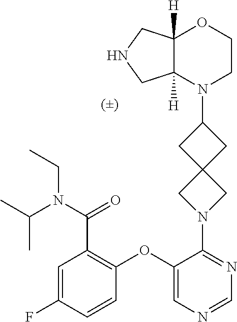 [SMILES: CCN(C(=O)c1cc(F)ccc1Oc1cncnc1N1CC2(CC(N3CCO[C@H]4CNC[C@@H]43)C2)C1)C(C)C]

This compound was synthesized following the procedure described for the synthesis of Example 32, step 4, starting with 1 equivalent of (±)-tert-butyl (4aS,7aS)-4-(2-(5-(2-(ethyl(isopropyl)carbamoyl)-4-fluorophenoxy)pyrimidin-4-yl)-2-azaspiro[3.3]heptan-6-yl)hexahydropyrrolo[3,4-b][1,4]oxazine-6(2H)-carboxylate. After completion, the reaction was concentrated under reduced pressure. The crude was purified by prep HPLC (Method F) to obtain (t)-N-ethyl-5-fluoro-2-((4-(6-((trans)-hexahydropyrrolo[3,4-b][1,4]oxazin-4(4aH)-yl)-2-azaspiro[3.3]heptan-2-yl)pyrimidin-5-yl)oxy)-N-isopropylbenzamide as a solid.

Yield: 55.2%; ¹H NMR. (400 MHz, DMSO-d₆): δ 8.34-8.20 (m, 1H), 7.79-7.65 (m, 1H), 7.36-7.18 (m, 2H), 7.07-6.92 (m, 1H), 4.25-3.93 (m, 5H), 3.90-3.83 (m, 1H), 3.81-3.67 (m, 1H), 3.60-3.48 (m, 2H), 3.43-3.40 ((m, 1H), 3.26-3.19 (m, 1H), 2.94-2.81 (m, 2H), 2.67-2.56 (m, 3H), 2.30-2.19 (m, 2H), 2.03-1.81 (m, 4H), 1.25-0.98 (m, 9H), one proton merged with solvent peaks; LCMS (Method D): Rt=1.34 min, m/z=525.3 [M+H]⁺; HPLC (Method A): Rt=3.96 min, 99.66%, Chiral SFC (Method L): Peak-1: Rt=1.97 min, 49.37%, Peak-2: 2.45 min, 50.63%.

### Example 36. 2-((4-(6-((2R,4R)-4-Amino-2-methylpiperidin-1-yl)-2-azaspiro[3.3]heptan-2-yl)pyrimidin-5-yl)oxy)-N-ethyl-5-fluoro-N-isopropylbenzamide (Compound No. 36)

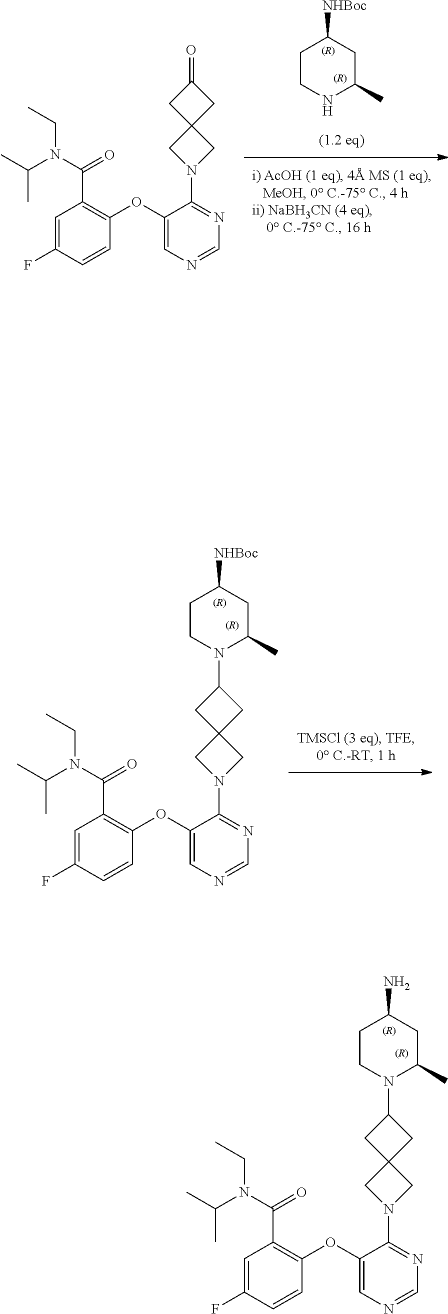

### Step 1. tert-Butyl ((2R,4R)-1-(2-(S-(2-(ethyl(isopropyl)carbamoyl)-4-fluorophenoxy)pyrimidin-4-yl)-2-azaspiro[3.3]heptan-6-yl)-2-methylpiperidin-4-yl)carbamate

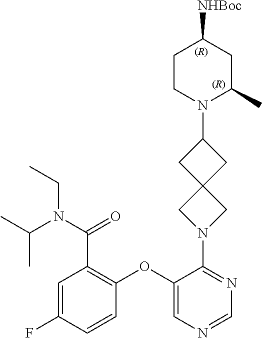

This compound was synthesized following the procedure described for the synthesis of Example 32, step 1, starting with 1 equivalent of N-ethyl-5-fluoro-N-isopropyl-2-((4-(6-oxo-2-azaspiro[3.3]heptan-2-yl)pyrimidin-5-yl)oxy)benzamide and 1.2 equivalents of tert-butyl ((2R,4R)-2-methylpiperidin-4-yl)carbamate, except that except that 4 equivalents of NaBH₃CN and t equivalent of AcOH were used, and MeOH was used as the solvent. The reaction was stirred at 75° C. After completion, the reaction was filtered through a Celite® pad, the pad was washed with MeOH, and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl ((2R,4R)-1-(2-(5-(2-(ethyl(isopropyl)carbamoyl)-4-fluorophenoxy)pyrimidin-4-yl)-2-azaspiro[3.3]heptan-6-yl)-2-methylpiperidin-4-yl)carbamate as a liquid.

Yield: 33.8%; LCMS (Method D): Rt=1.88 min, m/z=611.6 [M+H]⁺, 100%.

### Step 2. 2-((4-(6-((2R,4R)-4-Amino-2-methylpiperidine-1-yl)-2-azaspiro[3.3]heptan-2-yl)pyrimidin-5-yl)oxy)-N-ethyl-5-fluoro-N-isopropylbenzamide

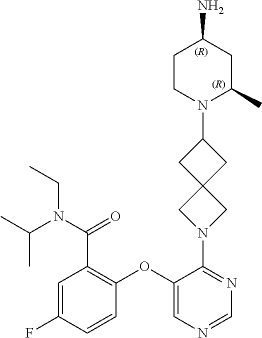

This compound was synthesized following the procedure described for the synthesis of Example 34, step 4, starting with 1 equivalent of tert-butyl ((2R,4R)-1-(2-(5-(2-(ethyl(isopropyl)carbamoyl)-4-fluorophenoxy)pyrimidin-4-yl)-2-azaspiro[3.3]heptan-6-yl)-2-methylpiperidin-4-yl)carbamate, except that 3 equivalents of TMSCl was used. After completion, the reaction was concentrated under reduced pressure. The crude was purified by prep HPLC (Method E) to obtain 2-((4-(6-((2R,4R)-4-amino-2-methylpiperidin-1-yl)-2-azaspiro[3.3]heptan-2-yl)pyrimidin-5-yl)oxy)-N-ethyl-5-fluoro-N-isopropylbenzamide as a solid.

Yield: 31.8%; ¹H NMR (400 MHz, DMSO-d₆): δ 8.32-8.23 (m, 11H), 7.79-7.65 (m, 1H), 7.33-7.17 (m, 2H), 7.07-6.95 (m, 1H), 4.25-4.01 (m, 3H), 3.99-3.92 (m, 1H), 3.75 (spt, J=6.6 Hz, 1H), 3.4-3.43 (m, 1H), 3.23-3.12 (m, 2H), 2.81-2.71 (m, 2H), 2.65-2.57 (m, 1H), 2.36-2.32 (m, 1H), 2.24-2.07 (m, 2H), 2.05-1.92 (m, 2H), 1.77-1.65 (m, 2H), 1.63-1.57 (m, 1H), 1.21-1.17 ((m, 3H), 1.13-0.92 (m, 12H); LCMS (Method B): Rt=1.30 min, m/z=511.4[M+H]⁺; HPLC (Method A): Rt=4.34 min, 97.50%; Chiral SFC (Method M): Rt=4.12 min, 100%.

### Example 37. 2-((4-(6-(4-Aminopiperidin-1-yl)-2-azaspiro[3.3]heptan-2-yl)pyrimidin-5-yl)oxy)-N-ethyl-5-fluoro-N-isopropylbenzamide (Compound No. 37)

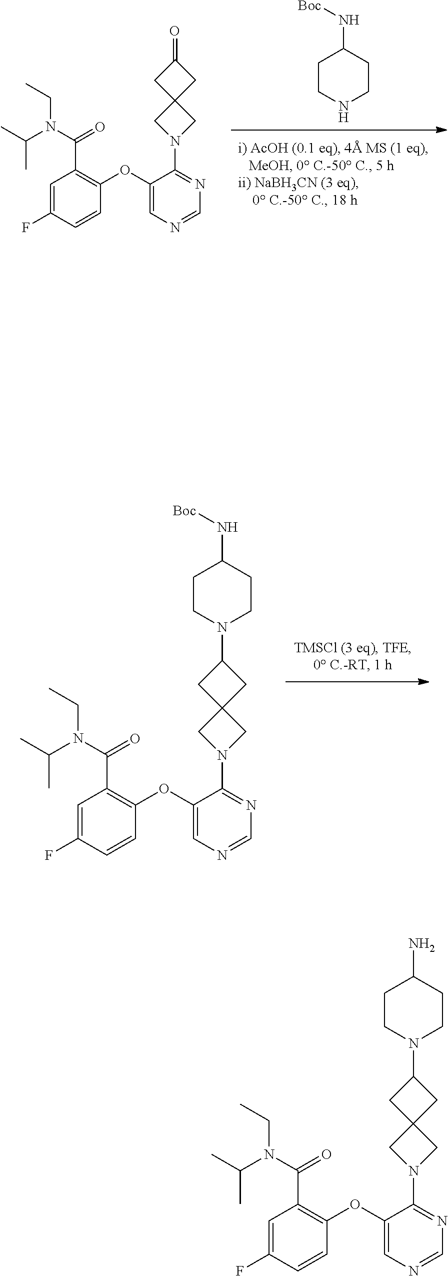 [SMILES: CCN(C(=O)c1cc(F)ccc1Oc1cncnc1N1CC2(CC(=O)C2)C1)C(C)C ; CC(C)(C)OC(=O)NC1CCNCC1 ; CCN(C(=O)c1cc(F)ccc1Oc1cncnc1N1CC2(CC(N3CCC(NC(=O)OC(C)(C)C)CC3)C2)C1)C(C)C ; CCN(C(=O)c1cc(F)ccc1Oc1cncnc1N1CC2(CC(N3CCC(N)CC3)C2)C1)C(C)C]

### Step 1. tert-Butyl (1-(2-(5-(2-(ethyl(isopropyl)carbamoyl)-4-fluorophenoxy)pyrimidin-4-yl)-2-azaspiro[3.3]heptan-6-yl)piperidin-4-yl)carbamate

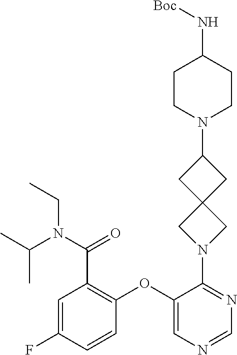 [SMILES: CCN(C(=O)c1cc(F)ccc1Oc1cncnc1N1CC2(CC(N3CCC(NC(=O)OC(C)(C)C)CC3)C2)C1)C(C)C]

This compound was synthesized following the procedure described for the synthesis of Example 32, step 1, starting with 1 equivalent of N-ethyl-5-fluoro-N-isopropyl-2-((4-(6-oxo-2-azaspiro[3.3]heptan-2-yl)pyrimidin-5-yl)oxy)benzamide and 1 equivalent of tert-butyl piperidin-4-ylcarbamate, except that 3 equivalents of NaBH₃CN and 0.1 equivalent of AcOH were used, and MeOH was used as the solvent. The reaction was stirred at 50° C. After work-up, the crude was purified by silica gel flash column chromatography using MeOH and DCM (product eluted at 7% MeOH in DCM) to obtain tert-butyl (1-(2-(5-(2-(ethyl(isopropyl)carbamoyl)-4-fluorophenoxy)pyrimidin-4-yl)-2-azaspiro[3.3]heptan-6-yl)piperidin-4-yl)carbamate as a solid.

Yield: 42.1%; LCMS (Method D): Rt=1.91 min, m/z=597.6 [M+H]⁺, 60.86%.

### Step 2. 2-((4-(6-(4-Aminopiperidin-1-yl)-2-azaspiro[3.3]heptan-2-yl)pyrimidin-5-yl)oxy)-N-ethyl-5-fluoro-N-isopropylbenzamide

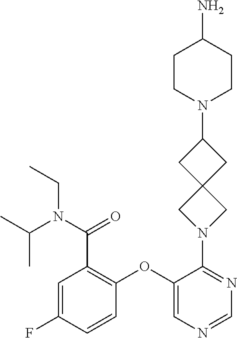 [SMILES: CCN(C(=O)c1cc(F)ccc1Oc1cncnc1N1CC2(CC(N3CCC(N)CC3)C2)C1)C(C)C]

This compound was synthesized following the procedure described for the synthesis of Example 34, step 4, starting with 1 equivalent of tert-butyl (1-(2-(5-(2-(ethyl(isopropyl)carbamoyl)-4-fluorophenoxy)pyrimidin-4-yl)-2-azaspiro[3.3]heptan-6-yl)piperidin-4-yl)carbamate, except that 3 equivalents of TMSCl was used. After completion, the reaction was concentrated under reduced pressure. The crude was purified by prep HPLC (Method L) to obtain 2-((4-(6-(4-aminopiperidin-1-yl)-2-azaspiro[3.3]heptan-2-yl)pyrimidin-5-yl)oxy)-N-ethyl-5-fluoro-N-isopropylbenzamide as a solid.

Yield: 38.5%; ¹H NMR (400 MHz, DMSO-d₆): δ 8.33-8.23 (m, 1H), 7.79-7.65 (m, 1H), 7.34-7.20 (m, 2H), 7.07-6.96 (m, 1H), 4.23-3.92 (m, 4H), 3.75 (spt, J=6.6 Hz, 1H), 3.46-3.38 (m, 1H), 3.27-3.10 (m, 2H), 2.66-2.60 (m, 2H), 2.30-2.18 (m, 2H), 1.97-1.86 (m, 2H), 1.75-1.59 (m, 4H), 1.23-0.97 (m, 11H), three protons merged with solvent peaks; LCMS (Method D): Rt=1.48 min, m/z=497.3 [M+H]⁺; HPLC (Method A): Rt=4.32 min, 99.00%.

### Example 38. (±)-2-((4-(6-(4-Amino-3,4-dihydroquinolin-1(2H)-yl)-2-azaspiro[3.3]heptan-2-yl)pyrimidin-5-yl)oxy)-N-ethyl-5-fluoro-N-isopropylbenzamide (Compound No. 38)

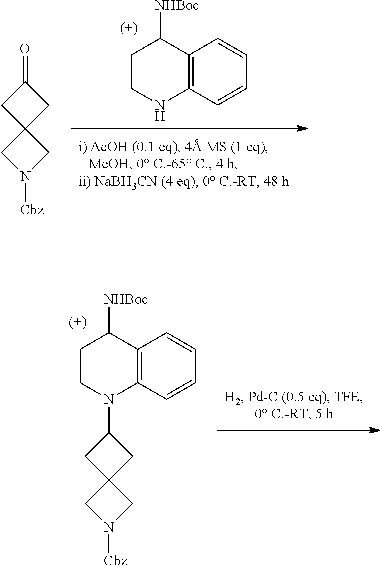 [SMILES: O=C1CC2(C1)CN(C(=O)OCc1ccccc1)C2 ; CC(C)(C)OC(=O)NC1CCNc2ccccc21 ; CC(C)(C)OC(=O)NC1CCN(C2CC3(C2)CN(C(=O)OCc2ccccc2)C3)c2ccccc21]

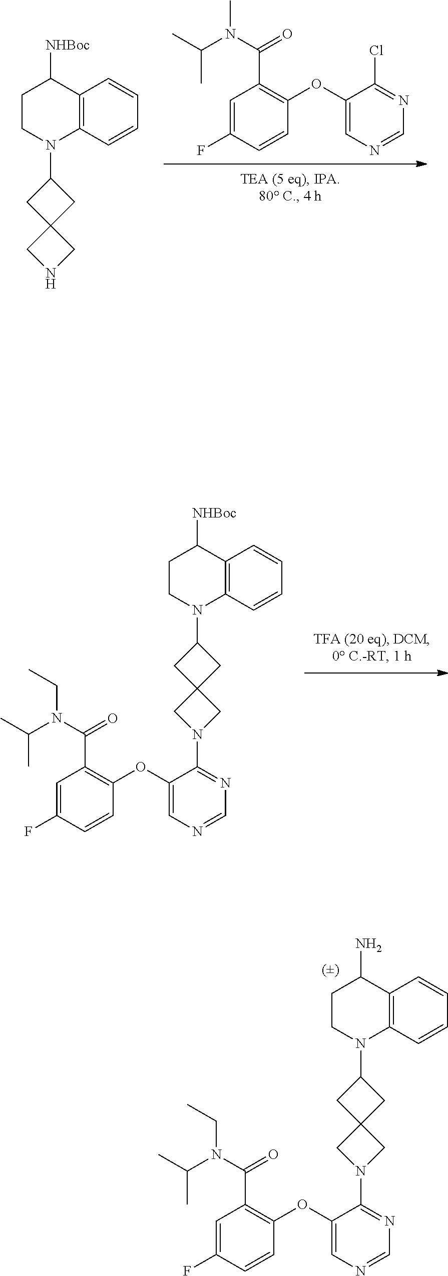 [SMILES: CC(C)(C)OC(=O)NC1CCN(C2CC3(CNC3)C2)c2ccccc21 ; CC(C)N(C)C(=O)c1cc(F)ccc1Oc1cncnc1Cl ; CCN(C(=O)c1cc(F)ccc1Oc1cncnc1N1CC2(CC(N3CCC(NC(=O)OC(C)(C)C)c4ccccc43)C2)C1)C(C)C ; CCN(C(=O)c1cc(F)ccc1Oc1cncnc1N1CC2(CC(N3CCC(N)c4ccccc43)C2)C1)C(C)C]

### Step 1. (±)-Benzyl 6-(4-((tert-butoxycarbonyl)amino)-3,4-dihydroquinolin-1 (2H)-yl)-2-azaspiro[3.3]heptane-2-carboxylate

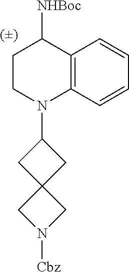 [SMILES: CC(C)(C)OC(=O)NC1CCN(C2CC3(C2)CN(C(=O)OCc2ccccc2)C3)c2ccccc21]

This compound was synthesized following the procedure described for the synthesis of Example 32, step 1, starting with 1 equivalent of benzyl 6-oxo-2-azaspiro[3.3]heptane-2-carboxylate and 1 equivalent of (±)-tert-butyl (1,2,3,4-tetrahydroquinolin-4-yl)carbamate, except that 4 equivalents of NaBH₃CN and 0.1 equivalent of AcOH were used, and MeOH was used as the solvent. The reaction was stirred at 50° C. After completion, the reaction was filtered through a Celite® pad, the pad was washed with MeOH, and the filtrate was concentrated under reduced pressure. The crude was purified by silica gel flash column chromatography using EtOAc and hexane (product eluted at 20% EtOAc in hexane) to obtain (±)-benzyl 6-(4-((tert-butoxycarbonyl)amino)-3,4-dihydroquinolin-1(2H)-yl)-2-azaspiro[3.3]heptane-2-carboxylate as a liquid.

Yield: 25.8%; LCMS (Method D): Rt=2.34 min, m/z=478.5 [M+H]⁺, 43.55%.

### Step 2. (±)-tert-Butyl (1-(2-azaspiro[3.3]heptan-6-yl)-1,2,3,4-tetrahydroquinolin-4-yl)carbamate

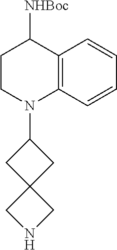 [SMILES: CC(C)(C)OC(=O)NC1CCN(C2CC3(CNC3)C2)c2ccccc21]

To a stirred solution of (±)-benzyl 6-(4-((tert-butoxycarbonyl)amino)-3,4-dihydroquinolin-1(2H)-yl)-2-azaspiro[3.3]heptane-2-carboxylate (300 mg, 0.628 mmol) in TFE (5 mL), Pd—C (334 mg, 0.314 mmol) was added at RT under a nitrogen atmosphere. The reaction was stirred at RT for 5 h under a hydrogen atmosphere (balloon pressure). The reaction progress was monitored by TLC (10% MeOH in DCM). After completion, the reaction was filtered through a Celite® pad, and the pad was washed with MeOH (20 mL). The filtrate was concentrated under reduced pressure to obtain crude (±)-tert-butyl (1-(2-azaspiro[3.3]heptan-6-yl)-1,2,3,4-tetrahydroquinolin-4-yl)carbamate as a gummy liquid (250 mg, 60.3% yield). This material was used without further purification.

LCMS (Method B): Rt=1.86 min, m/z=344.3 [M+H]⁺, 52.08%.

### Step 3. (±)-tert-Butyl (1-(2-(5-(2-(ethyl(isopropyl)carbamoyl)-4-fluorophenoxy)pyrimidin-4-yl)-2-azaspiro[3.3]heptan-6-yl)-1,2,3,4-tetrahydroquinolin-4-yl)carbamate

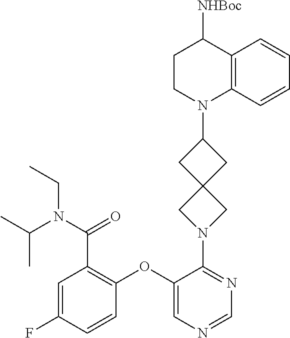 [SMILES: CCN(C(=O)c1cc(F)ccc1Oc1cncnc1N1CC2(CC(N3CCC(NC(=O)OC(C)(C)C)c4ccccc43)C2)C1)C(C)C]

This compound was synthesized following the procedure described for the synthesis of Example 30, step 3, starting with 1 equivalent of (±)-tert-butyl (1-(2-azaspiro[3.3]heptan-6-yl)-1,2,3,4-tetrahydroquinolin-4-yl)carbamate and 1 equivalent of 2-((4-chloropyrimidin-5-yl)oxy)-N-ethyl-5-fluoro-N-isopropylbenzamide, except that 5 equivalents of TEA was used. After completion, brine solution was added, and the mixture was extracted with EtOAc. The combined organic layer was washed with brine solution, dried over anhydrous sodium sulfate and filtered, and the filtrate was concentrated under reduced pressure. The crude was purified by silica gel flash column chromatography using MeOH in DCM (product eluted at 5% MeOH in DCM) to obtain (±)-tert-butyl (I-(2-(5-(2-(ethyl(isopropyl)carbamoyl)-4-fluorophenoxy)pyrimidin-4-yl)-2-azaspiro[3.3]heptan-6-yl)-1,2,3,4-tetrahydroquinolin-4-yl)carbamate as a semi-solid.

Yield: 38.8%; LCMS (Method D): Rt=2.28 min, m/z=645.3 [M+H]⁺, 91.63%.

### Step 4. (±)-2-((4-(6-(4-Amino-3,4-dihydroquinolin-1(2H)-yl)-2-azaspiro/3.3/heptan-2-yl)pyrimidin-5-yl)oxy)-N-ethyl-5-fluoro-N-isopropylbenzamide

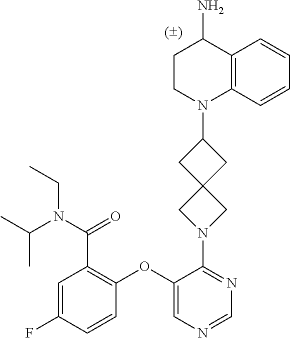 [SMILES: CCN(C(=O)c1cc(F)ccc1Oc1cncnc1N1CC2(CC(N3CCC(N)c4ccccc43)C2)C1)C(C)C]

This compound was synthesized following the procedure described for the synthesis of Example 32, step 4, starting with 1 equivalent of (±)-tert-butyl (1-(2-(5-(2-(ethyl(isopropyl)carbamoyl)-4-fluorophenoxy)pyrimidin-4-yl)-2-azaspiro[3.3]heptan-6-yl)-1,2,3,4-tetrahydroquinolin-4-yl)carbamate, except that 20 equivalents of TFA was used. After completion, the reaction was concentrated under reduced pressure. The crude was purified by prep HPLC (Method F) to obtain (±)-2-((4-(6-(4-amino-3,4-dihydroquinolin-1(2H)-yl)-2-azaspiro[3.3]heptan-2-yl)pyrimidin-5-yl)oxy)-N-ethyl-5-fluoro-N-isopropylbenzamide as a solid.

Yield: 41.8%; ¹H NMR (400 MHz, DMSO-d₆): δ 8.33-8.26 (m, 1H), 7.77-7.66 (m, 1H), 7.34-7.22 (m, 2H), 7.21-7.17 (m, 1H), 7.11-6.96 (m, 2H), 6.61-6.53 (m, 2H), 4.36-4.21 (m, 2H), 4.18-4.02 ((m, 2H), 3.96-3.83 (m, 1H), 3.80-3.71 (m, 2H), 3.46-3.38 (m, 1H), 3.28-3.21 (m, 1H), 3.19-3.05 (m, 3H), 2.32-2.24 (m, 2H), 1.94-1.77 (m, 2H), 1.68-1.57 (m, 1H), 1.24-0.97 (m, 9H), one proton merged with solvent peaks; LCMS (Method D): Rt=1.77 min, m/z 545.0 [M+H]⁺; HPLC (Method D): Rt=3.07 min, 97.08%; Chiral SFC (Method N): Peak-1: 3.59 min, 42.81%, Peak-2: 3.93 min, 56.58%.

### Example 39. (E1)-2-((4-(6-(4-Amino-3,4-dihydroquinolin-1(2H)-yl)-2-azaspiro[3.3]heptan-2-yl)pyrimidin-5-yl)oxy)-N-ethyl-5-fluoro-N-isopropylbenzamide (Compound No. 39)

### Example 40. (E2)-2-((4-(6-(4-Amino-3,4-dihydroquinolin-1(2H)-yl)-2-azaspiro[3.3]heptan-2-yl)pyrimidin-5-yl)oxy)-N-ethyl-5-fluoro-N-isopropylbenzamide (Compound No. 40)

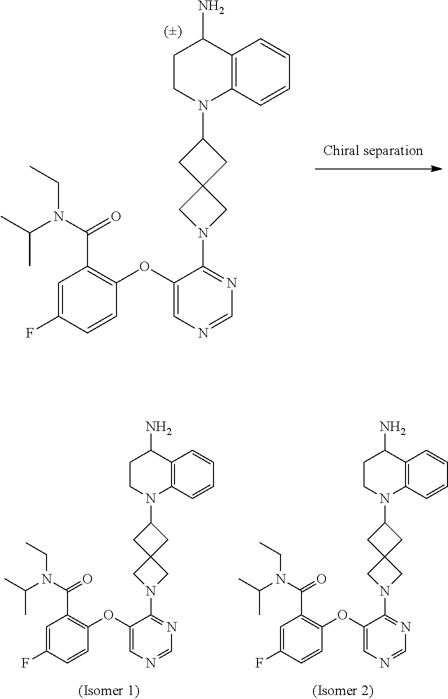 [SMILES: CCN(C(=O)c1cc(F)ccc1Oc1cncnc1N1CC2(CC(N3CCC(N)c4ccccc43)C2)C1)C(C)C ; CCN(C(=O)c1cc(F)ccc1Oc1cncnc1N1CC2(CC(N3CCC(N)c4ccccc43)C2)C1)C(C)C ; CCN(C(=O)c1cc(F)ccc1Oc1cncnc1N1CC2(CC(N3CCC(N)c4ccccc43)C2)C1)C(C)C]

(±)-2-((4-(6-(4-Amino-3,4-dihydroquinolin-1(2H)-yl)-2-azaspiro[3.3]heptan-2-yl)pyrimidin-5-yl)oxy)-N-ethyl-5-fluoro-N-isopropylbenzamide (50 mg) was purified by Chiral prep SFC (Method E) to obtain both isomers.

Isomer 1: (E1)-2-((4-(6-(4-Amino-3,4-dihydroquinolin-1(2H)-yl)-2-azaspiro[3.3]heptan-2-yl)pyrimidin-5-yl)oxy)-N-ethyl-5-fluoro-N-isopropylbenzamide (9 mg, 17.10% yield) as a solid.

¹H NMR (400 MHz, DMSO-d₆): δ 8.32-8.25 (m, 1H), 7.81-7.64 ((m, 1H), 7.33-7.18 (m, 3H), 7.14-7.01 (m, 2H), 6.69-6.59 (m, 2H), 4.37-4.22 (m, 2H), 4.18-4.02 (m, 3H), 3.98-3.88 (m, 1H), 3.82-3.70 (m, 1H), 3.47-3.41 (m, 1H), 3.30-3.00 (m, 4H), 2.64-2.57 (m, 1H), 2.33-2.24 (m, 2H), 2.03-1.95 (m, 1H), 1.94-1.81 (m, 1H), 1.22-0.97 (m, 9H), two protons merged with solvent peaks: LCMS (Method B): Rt=1.73 min, m/z=545.5 [M+H]⁺; HPLC (Method A): Rt=5.47 min, 95.00%; Chiral SFC (Method P): 6.12 min, 94.43%.

Isomer 2: (E2)-2-((4-(6-(4-Amino-3,4-dihydroquinolin-1(2H)-yl)-2-azaspiro[3.3]heptan-2-yl)pyrimidin-5-yl)oxy)-N-ethyl-5-fluoro-N-isopropylbenzamide (5 mg, 9.78% yield) as a solid.

¹H NMR (400 MHz, DMSO-d₆): δ 8.32-8.26 (m, 1H), 7.81-7.65 (m, 1H), 7.34-7.18 (m, 3H), 7.14-7.00 (m, 2H), 6.69-6.59 (m, 2H), 4.37-4.21 (m, 2H), 4.18-4.01 (m, 3H), 3.97-3.89 (m, 1H), 3.81-3.72 (m, 1H), 3.48-3.42 (m, 1H), 3.31-3.01 (m, 4H), 2.63-2.58 (m, 1H), 2.32-2.22 (m, 2H), 2.04-1.94 (m, 1H), 1.92-1.82 (m, 1H), 1.21-0.97 (m, 9H), two protons merged with solvent peaks; LCMS (Method B): Rt=1.72 min, m/z=545.6[M+H]⁺; HPLC (Method A): Rt=5.47 min, 97.79%; Chiral SFC (Method P): 7.31 min, 100K.

### Example 41. 5-Fluoro-N,N-diisopropyl-2-((4-(6-(((4aS,8aS)-octahydro-4H-pyrido[4,3-b][1,4]oxazin-4-yl)methyl)-2-azaspiro[3,4]octan-2-yl)pyrimidin-5-yl)oxy)benzamide (Compound No. 41)

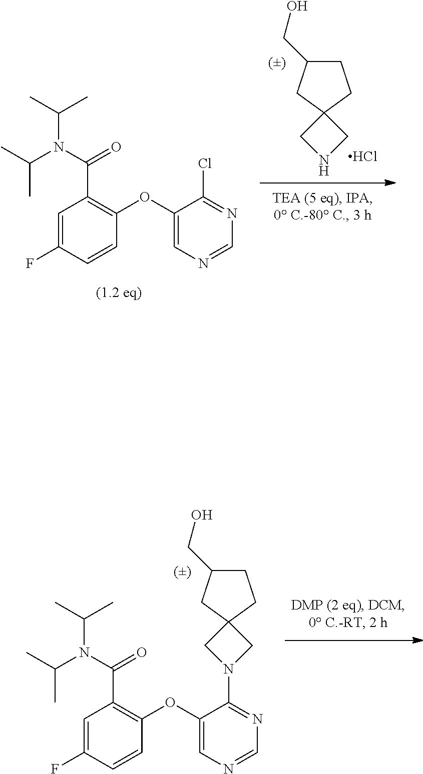 [SMILES: CC(C)N(C(=O)c1cc(F)ccc1Oc1cncnc1Cl)C(C)C ; OCC1CCC2(CNC2)C1 ; Cl ; CC(C)N(C(=O)c1cc(F)ccc1Oc1cncnc1N1CC2(CCC(CO)C2)C1)C(C)C]

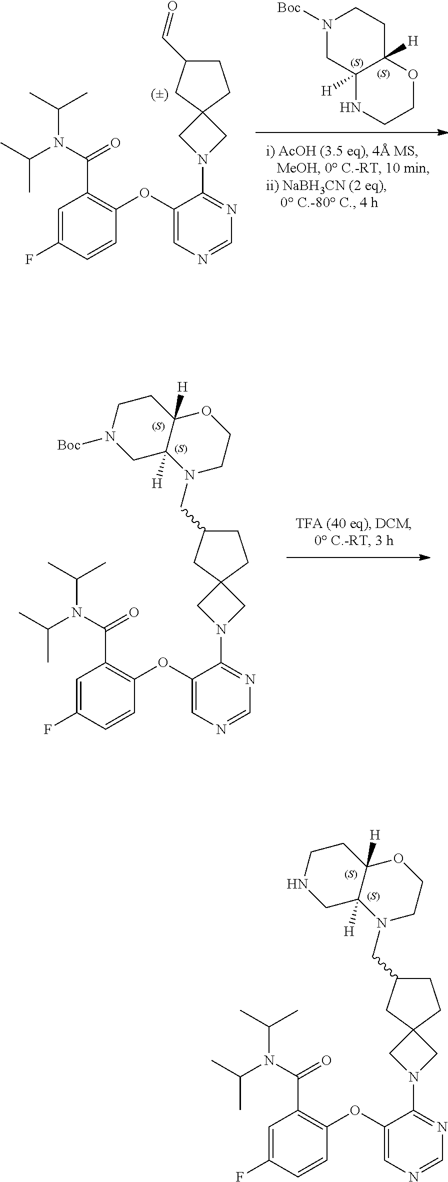

### Step 1. (±)-5-Fluoro-2-((4-(6-(hydroxymethyl)-2-azaspiro[3.4]octan-2 yl)pyrimidin-5-yl)oxy)-N,N-diisopropylbenzamide

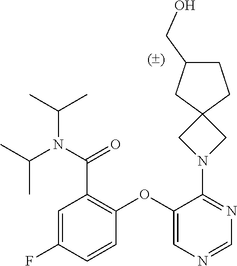 [SMILES: CC(C)N(C(=O)c1cc(F)ccc1Oc1cncnc1N1CC2(CCC(CO)C2)C1)C(C)C]

This compound was synthesized following the procedure described for the synthesis of Example 30, step 3, starting with 1 equivalent of (±)-(2-azaspiro[3.4]octan-6-yl)methanol hydrochloride and 1.2 equivalents of 2-((4-chloropyrimidin-5-yl)oxy)-5-fluoro-N,N-diisopropylbenzamide, except that 5 equivalents of TEA was used. After completion, the reaction was quenched with brine solution and the mixture was extracted with EtOAc. The combined organic layer was washed with brine solution, dried over anhydrous sodium sulfate and filtered, and the filtrate was concentrated under reduced pressure. The crude was purified by silica gel flash column chromatography using MeOH in DCM (product eluted at 5% MeOH in DCM) to obtain (±)-5-fluoro-2-((4-(6-(hydroxymethyl)-2-azaspiro[3.4]octan-2-yl)pyrimidin-5-yl)oxy)-N,N-diisopropylbenzamide as a semi-solid.

Yield: 77%; LCMS (Method D): Rt=1.74 min, m/z=457.4 [M+H]⁺, 74.55%.

### Step 2. (±)-S-Fluoro-2-((4-(6-formyl-2-azaspiro[3.4]octan-2-yl)pyrimidin-5-yl)oxy)-N,N-diisopropylbenzamide

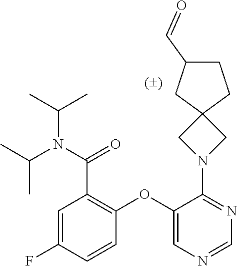 [SMILES: CC(C)N(C(=O)c1cc(F)ccc1Oc1cncnc1N1CC2(CCC(C=O)C2)C1)C(C)C]

To a stirred solution of (±)-5-fluoro-2-((4-(6-(hydroxymethyl)-2-azaspiro[3,4]octan-2-yl)pyrimidin-5-yl)oxy)-N,N-diisopropylbenzamide (0.53 g, 1.161 mmol) in DCM (15 mL), DMP (0.985 g, 2.322 mmol) was added at 0° C. under a nitrogen atmosphere. The reaction was stirred at the same temperature for 15 min, then at RT for 2 h, and monitored by TLC (100% EtOAc). After completion, the reaction was quenched with saturated sodium bicarbonate solution (100 mL), and the mixture was extracted with DCM (2×100 mL). The combined organic layer was washed with NaHCO₃ solution (3×100 mL), dried over anhydrous sodium sulfate and filtered, and the filtrate was concentrated under reduced pressure to afford crude (±)-5-fluoro-2-((4-(6-formyl-2-azaspiro[3.4]octan-2-yl)pyrimidin-5-yl)oxy)-N,N-diisopropylbenzamide (0.55 g, quantitative yield) as a gummy liquid. This material was used without further purification.

### Step 3. tert-Butyl (4aS,8aS,)-4-((2-(5-(2-(diisopropylcarbamoyl)-4-fluorophenoxy)pyrimidin-4-yl)-2-azaspiro[3.4]octan-yl)methyl)hexahydro-2H-pyrido[4,3-b][1,4]oxazine-6(5H)-carboxylate

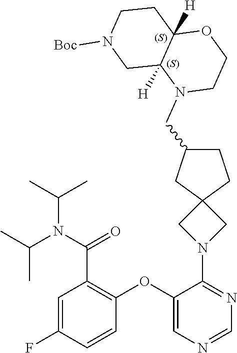

To a stirred solution of (±)-tert-butyl (4aS,8aS)-hexahydro-2H-pyrido[4,3-b][1,4]oxazine-6(5H)-carboxylate (0.3 g, 1.238 mmol) and 5-fluoro-2-((4-(6-formyl-2-azaspiro[3.4]octan-2-yl)pyrimidin-5-yl)oxy)-N,N-diisopropylbenzamide (0.563 g, 1.238 mmol) in MeOH (25 mL), AcOH (0.248 mL, 4.33 mmol) and 4 Å MS were added at RT under a nitrogen atmosphere. The reaction mixture was stirred at the same temperature for 10 min, then NaBH₃CN (0.156 g, 2.476 mmol) was added. The reaction was stirred at RT for 10 min, then at 80° C. for 4 h. The reaction progress was monitored by TLC (10% MeOII in DCM). After completion, the reaction was filtered through a Celite® pad, the pad was washed with MeOH (25 mL), and the filtrate was concentrated under reduced pressure. The crude was purified by reverse phase chromatography using a SiliCycle SiliaSep C18 column (80 g) on Biotage isolera one (Mobile phase A: 10 mM NH₄HCO₃ in water, B: ACN) to obtain tert-butyl (4aS,8aS)-4-((2-(5-(2-(diisopropylcarbamoyl)-4-fluorophenoxy)pyrimidin-4-yl)-2-azaspiro[3.4]octan-6-yl)methyl)hexahydro-2H-pyrido[4,3-b][1,4]oxazine-6(5N)-carboxylate (0.309 g, 35.0% yield) as a solid. ¹H NMR (400 MHz, DMSO-d₆): δ 8.28-8.24 (m, 1H), 7.77-7.67 (m, 1H), 7.27-7.18 (m, 2H), 7.10-6.99 (m, 1H), 4.38-4.17 (m, 1H), 4.13-3.89 (m, 5H), 3.80-3.64 (m, 2H), 3.60-3.51 (m, 2H), 3.20-3.11 (m, 1H), 2.85-2.73 (m, 2H), 2.46-2.39 (m, 2H), 2.22-2.06 (m, 3H), 2.00-1.91 (m, 1H), 1.87-1.65 (m, 6H), 1.43 (d, J=6.8 Hz, 3H), 1.40-1.37 (m, 9H), 1.35 (hr d, J=6.6 Hz. 3H), 1.09 (d, J=6.6 Hz, 3H), 1.00 (br d, J=6.3 Hz, 3H), two protons merged with solvent peaks; LCMS (Method D): Rt=2.27 min, m/z=681.4 [M+H]⁺, 97.23%.

### Step 4. 5-Fluoro-N,N-diisopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide

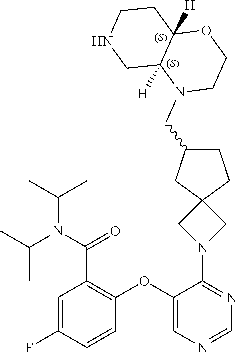

This compound was synthesized following the procedure described for the synthesis of Example 32, step 4, starting with 1 equivalent of tert-butyl (4aS,8aS)-4-((2-(5-(2-(diisopropylcarbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate, except that 40 equivalents of TFA was used. After completion, the reaction was concentrated under reduced pressure. The crude was purified by prep HPLC (Method R) to obtain 5-fluoro-N,N-diisopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide as a solid.

Yield: 45.1%; ¹H NMR (400 MHz, DMSO-d₆): δ8.31-8.24 (m, 1H), 7.76-7.69 (m, 1H), 7.27-7.19 (m, 2H), 7.07-6.99 (m, 1H), 4.11-3.89 (m, 4H), 3.74-3.64 (m, 2H), 3.57-3.48 (m, 2H), 3.23-3.16 (m, 1H), 3.07-2.97 (m, 1H), 2.90-2.74 (m, 2H), 2.48-2.38 (m, 2H), 2.20-2.00 (m, 3H), 1.98-1.85 (m, 2H), 1.84-1.59 (m, 5H), 1.55-1.48 (m, 1H), 1.44 (d, J=6.7 Hz, 3H), 1.35 (d, J=6.7 Hz, 3H), 1.33-1.22 (m, 2H), 1.20-1.13 (m, 1H), 1.09 (d, J=6.6 Hz, 3H), 1.03-0.96 (m, 3H); LCMS (Method B): Rt=1.55 min, m/z=581.4 [M+H]⁺; HPLC (Method E): Rt=3.24 min, 99.85%; Chiral SFC (Method S): Peak 1: Rt=6.16 min, 41.01%, Peak 2: Rt=6.60 min, 58.99%.

### Example 42. (D1)-5-Fluoro-N,N-diisopropyl-2-((4-(6-(((4aS,8aS)-octahydro-4H-pyrido[4,3-b][1,4]oxazin-4-yl)methyl)-2-azaspiro[3.4]octa-2-yl)pyrimidin-5-yl)oxy)benzamide (Compound No. 42)

### Example 43. (D2)-5-Fluoro-N,N-diisopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide (Compound No. 43)

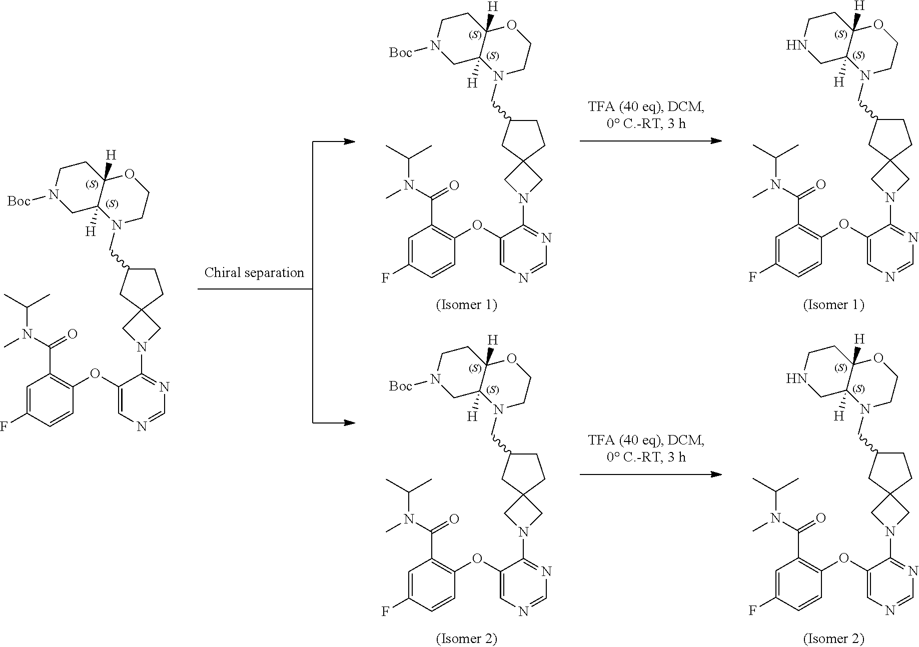

### Step 1. (D1)-tert-Butyl (4aS,8aS)-4-((2-(5-(2-(diisopropylcarbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate and (D2)-tert-butyl (4aS,8aS)-4-((2-(5-(2-(diisopropylcarbamoyl)-4-fluorophenoxy)pyrimidin-4-yl)-2-azaspiro[3.4]octan-6-yl)methyl)hexahydro-2)-carboxylate

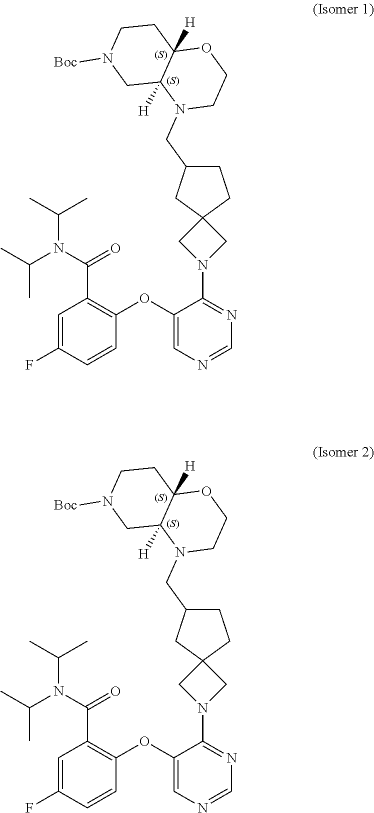

The diastereomeric mixture of tert-butyl (4aS,8aS)-4-((2-(5-(2-(diisopropylcarbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate (129 mg) was purified by Chiral prep SFC (Method G) to obtain both isomers.

Isomer 1: (D1)-tert-Butyl (4aS,8aS)-4-((2-(5-(2-(diisopropylcarbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate (0.045 g, 29.3% yield) as a solid. LCMS (Method D): Rt=2.28 min, m/z=681.4 [M+H]⁺, 83.70%; Chiral SFC (Method A1): Rt=4.47 min, 100%.

Isomer 2: (D2)-tert-Butyl (4aS,8aS)-4-((2-(5-(2-(diisopropylcarbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate (0.050 g, 37.2% yield) as a solid. LCMS (Method D): Rt=2.26 min, m/z=681.4 [M+H]⁺, 96.43%; Chiral SFC (Method A1): Rt=4.99 min, 100%.

### Step 2. (D1)-5-Fluoro-N,N-disopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide

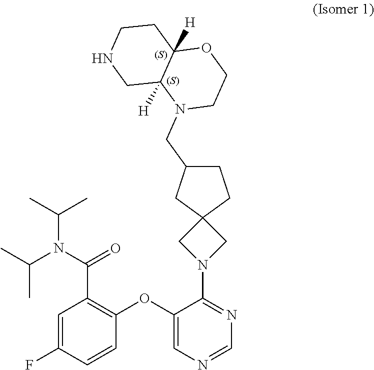

This compound was synthesized following the procedure described for the synthesis of Example 32, step 4, starting with 1 equivalent of (D1)-tert-Butyl (4aS,8aS)-4-((2-(5-(2-(diisopropylcarbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate, except that 40 equivalents of TFA was used. After completion, the reaction was concentrated under reduced pressure. The crude was purified by prep HPLC (Method S) to obtain (D1)-5-fluoro-N,N-diisopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide as a solid.

Yield: 65.0%; ¹H NMR (400 MHz, DMSO-d₆): δ 8.31-8.24 (m, 1H), 7.77-7.69 (m, 1H), 7.28-7.18 (m, 2H), 7.07-6.98 (m, 1H), 4.12-3.89 (m, 4H), 3.77-3.63 (m, 2H), 3.59-3.47 (m, 2H), 3.22-3.15 (m, 1H), 3.08-2.95 (m, 1H), 2.90-2.83 (m, 1H), 2.81-2.75 (m, 1H), 2.47-2.38 (m, 2H), 2.17-2.03 (m, 3H), 1.99-1.85 (m, 3H), 1.84-1.68 (m, 4H), 1.65-1.60 (m, 1H), 1.56-1.47 (m, 1H), 1.44 (d, J=6.6 Hz, 3H), 1.35 (d, J=6.6 Hz, 3H), 1.33-1.24 (m, 1H), 1.22-1.13 (m, 1H), 1.09 (d, J=6.5 Hz, 3H), 1.03-0.97 (m, 3H); LCMS (Method C): Rt=1.69 min, m/z=581.4 [M+H]⁺; HPLC (Method E): Rt=3.16 min, 99.87%; Chiral SFC (Method S): 5.77 min, 100%.

### Step 3. (D2)-S-Fluoro-N,N-diisopropyl-2-((4-(6-(((4aS,8aS)-octahydro-4H-pyrido[4,3-b][1,4]oxazin-4-yl)methy)-2-azaspiro[3.4]octan-2-yl)pyrimidin-5-yl)oxy)benzamide

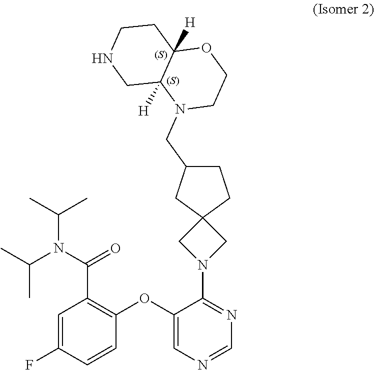

This compound was synthesized following the procedure described for the synthesis of Example 32, step 4, starting with 1 equivalent of (D2)-tert-Butyl (4aS,8aS)-4-((2-(5-(2-(diisopropylcarbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate, except that 40 equivalents of TFA was used. After completion, the reaction was concentrated under reduced pressure. The crude was purified by prep HPLC (Method C) to obtain (D2)-5-fluoro-N,N-diisopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide as a solid.

Yield: 58.5%; ¹H NMR (400 MHz, DMSO-d₆): δ 8.27-8.26 (m, 1H), 7.74-7.71 (m, 1H), 7.26-7.19 (m, 2H), 7.06-7.00 (m, 1H), 4.14-3.88 (m, 4H), 3.77-3.63 (m, 2H), 3.58-3.49 (m, 2H), 3.24-3.16 (m, 1H), 3.07-2.98 (m, 1H), 2.93-2.84 (m, 1H), 2.81-2.74 (m, 1H), 2.47-2.40 (m, 2H), 2.18-2.00 (m, 4H), 1.99-1.89 (m, 2H), 1.83-1.60 (m, 5H), 1.44 (d, J=6.7 Hz, 3H), 1.42-1.38 (m, 1H), 1.35 (d, J=6.7 Hz, 3H), 1.33-1.21 (m, 2H), 1.09 (d, J=6.6 Hz, 3H), 1.03-0.96 (m, 3H); LCMS (Method B): Rt=1.57 min, m/z=581.4 [M+H]⁺; HPLC (Method D): Rt=2.45 min, 99.81%; Chiral SFC (Method S): 6.45 min, 100%.

### Example 44. (D1)-N-Ethyl-5-fluoro-N-isopropyl-2-((4-(6-(((4aS,8aS)-octahydro-1H-pyrido[3,4-b][1,4]oxazin-1-yl)methyl)-2-azaspiro[3.4]octan-2-yl)pyrimidin-5-yl)oxy)benzamide (Compound No. 44)

### Example 45. (D2)-N-Ethyl-5-fluoro-N-isopropyl-2-((4-(6-(((4aS,8aS)-octahydro-1H-pyrido[3,4-b][1,4]oxazin-1-yl)methyl)-2-azaspiro[3.4]octan-2-yl)pyrimidin-5-yl)oxy)benzamide (Compound No. 45)

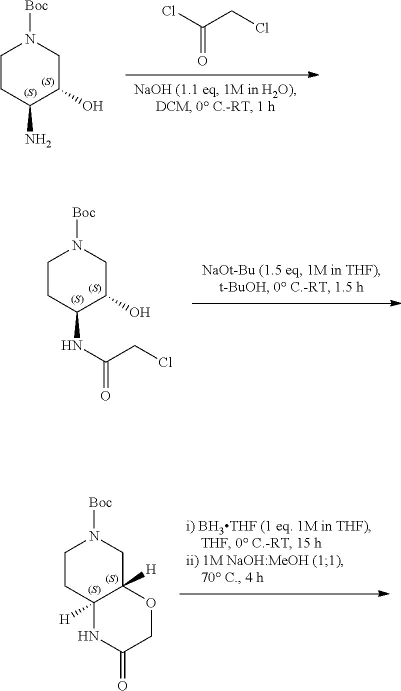

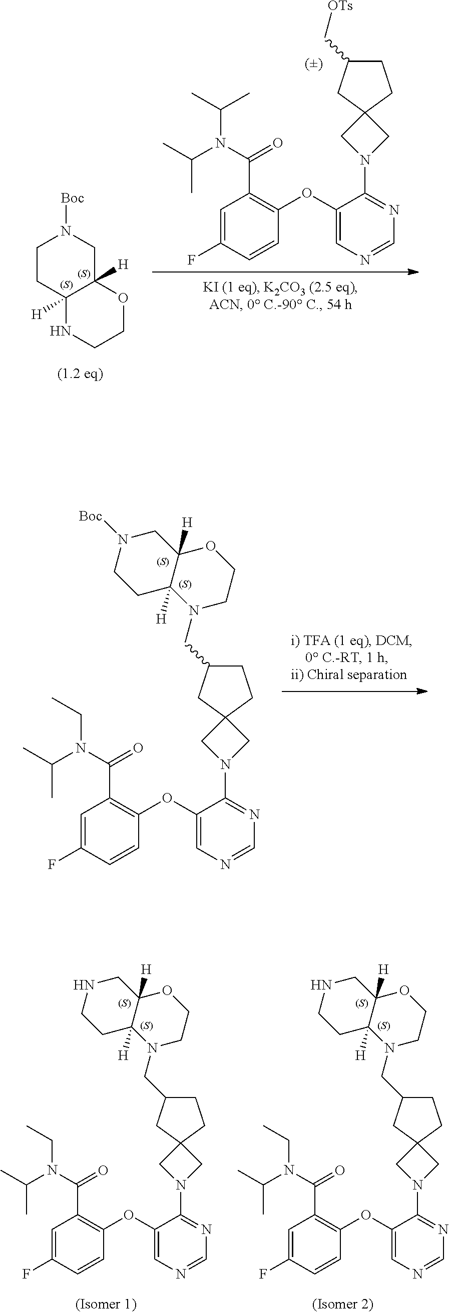

### Step 1. tert-Butyl (3S,4S)-4-(2-chloroacetamide 3-hydroxypiperidine-1-carboxylate

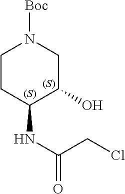

This compound was synthesized following the procedure described for the synthesis of Intermediate 7, step 1, starting with 1 equivalent of tert-butyl (3S,4S)-4-amino-3-hydroxypiperidine-1-carboxylate and 1 equivalent of 2-chloroacetyl chloride. After work-up, the organic layer was concentrated under reduced pressure to obtain crude tert-butyl (3S,4S)-4-(2-chloroacetamido)-3-hydroxypiperidine-1-carboxylate as a liquid. This material was used without further purification.

Yield: 68.7%; ¹H NMR (400 MHz, DMSO-d₆): δ 8.09 (br d, J=7.9 Hz, 11H), 5.05 (d, J=5.1 Hz, 1H), 4.00-3.90 (m, 1H), 3.83-3.723 (m, 1H), 3.65-3.52 (m, 1H), 3.29-3.18 (m, 1H), 2.86-2.74 (m, 1H), 2.59-2.53 (m, 2H), 1.85-1.67 (m, 1H), 1.40 (s, 9H), 1.33-1.21 (m, 1H), one proton merged with solvent peaks; LCMS (Method D): Rt=1.33 min, m/z=193.0 [M+H−100]⁺, 91.12%;

### Step 2. tert-Butyl (4aS,8aS)-2-oxooctahydro-6H-pyrido[3,4-b][1,4]oxazine-6-carboxylate

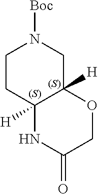

This compound was synthesized following the procedure described for the synthesis of Intermediate 7, Step 2, starting with 1 equivalent of tert-butyl (3S,4S)-4-(2-chloroacetamido)-3-hydroxypiperidine-1-carboxylate. After work-up, the crude was purified by silica gel flash column chromatography using EtOAc in hexane (product eluted at 30% EtOAc in hexane) to obtain tert-butyl (4aS,8aS)-2-oxooctahydro-6H-pyrido[3,4-b][1,4]oxazine-6-carboxylate as a solid.

Yield: 91%; ¹H NMR (400 MHz, DMSO-d₆): δ 8.26 (s, 1H), 4.10 (s, 2H), 4.07-3.90 (m, 2H), 3.25-3.16 (m, 2H), 2.84-2.59 (m, 2H), 1.89-1.78 (m, 1H), 1.40 (s, 9H), 1.29-1.17 (m, 1H); LCMS (Method B): Rt=1.86 min, m/z=257.3 [M+H]⁺, 99.27%.

### Step 3. tert-Butyl (4aS,8aS)-octahydro-6H-pyrido[3,4-b][1,4]oxazine-6-carboxylate

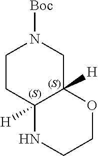

This compound was synthesized following the procedure described for the synthesis of Intermediate 7, Step 3, starting with 1 equivalent of tert-butyl (4aS,8aS)-2-oxooctahydro-6H-pyrido[3,4-b][1,4]oxazine-6-carboxylate. After work-up, the organic layer was concentrated under reduced pressure to obtain crude tert-butyl (4aS,8aS)-octahydro-6H-pyrido[3,4-b][1,4]oxazine-6-carboxylate as a semi-solid. This material was used without further purification.

Yield: 54.5%; LCMS (Method D): Rt=1.39 min, m/z=243.1 [M+H]⁺, 85.80%.

### Step 4. tert-Butyl (4aS,8aS)-1-((2-(5-(2-(ethyl(isopropyl)carbamoyl)-4-fluorophenoxy)pyrimidin-4-yl)-2-azaspiro[3.4]octan-6-yl)methyl)octahydro-6H-pyrido[3,4-b][1.4]oxazine-6-carboxylate

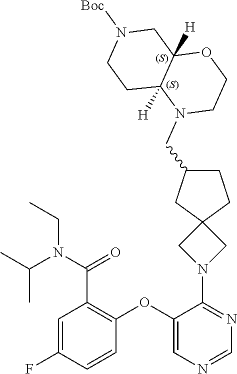

In a 4 mL pressure vial, (±)-(2-(5-(2-(ethyl(isopropyl)carbamoyl)-4-fluorophenoxy)pyrimidin-4-yl)-2-azaspiro[3.4]octan-6-yl)methyl-4-methylbenzenesulfonate (200 mg, 0.335 mmol)) and tert-butyl (4aS,8aS)-octahydro-6H-pyrido[3,4-b][1,4]oxazine-6-carboxylate (97 mg, 0.402 mmol) were dissolved in ACN (4 mL). KI (55.6 mg, 0.335 mmol) and K₂CO₃ (116 mg, 0.838 mmol) were then added at RT, and the reaction was stirred at 90° C. for 36 h. Based on the TLC and LCMS, the reaction was not complete. Therefore, another portion of tert-butyl (4aS,8aS)-octahydro-6H-pyrido[3,4-b][1,4]oxazine-6-carboxylate (72 mg, 0.402 mmol) was added at RT and the reaction was stirred at 90° C. for 18 h. The reaction progress was monitored by TLC (100% EtOAc). After completion, the reaction was quenched with water (25 mL) and extracted with EtOAc (2×25 mL). The combined organic layer was washed with brine solution (50 mL), dried over anhydrous sodium sulfate and filtered, and the filtrate was concentrated under reduced pressure. The crude product was purified by silica gel flash column chromatography using MeOH in DCM (product eluted at 10% MeOH in DCM) to obtain mixture of diastereomers of tert-butyl (4aS,8aS)-1-((2-(5-(2-(ethyl(isopropyl)carbamoyl)-4-fluorophenoxy)pyrimidin-4-yl)-2-azaspiro[3.4]octan-6-yl)methyl)octahydro-6H-pyrido[3,4-b][1,4]oxazine-6-carboxylate (180 mg, 56.9% yield) as a sticky liquid. LCMS (Method D): Rt=3.01 min, m/z=667.7 [M+H]⁺, 70.65%.

### Step 5. (D1)-N-Ethyl-5-fluoro-N-isopropyl-2-((4-(6-(((4aS,8aS)-octahydro-1H-pyrrolo[3,4-b][1,4]oxazin-yl)methyl)-2-azaspiro[3.4]octan-2-yl)pyrimidin-5-yl)oxy)benzamide and (D2)-N-ethyl-5-fluoro-N-isopropyl-2-((4-(6-(((4aS,8aS)-octahydro-1H-pyrido[3,4-b][1,4]oxazin-1-ylmethyl)-2-azaspiro[3.4]octan-2-yl)pyrimidin-5-yl)oxy)benzamide

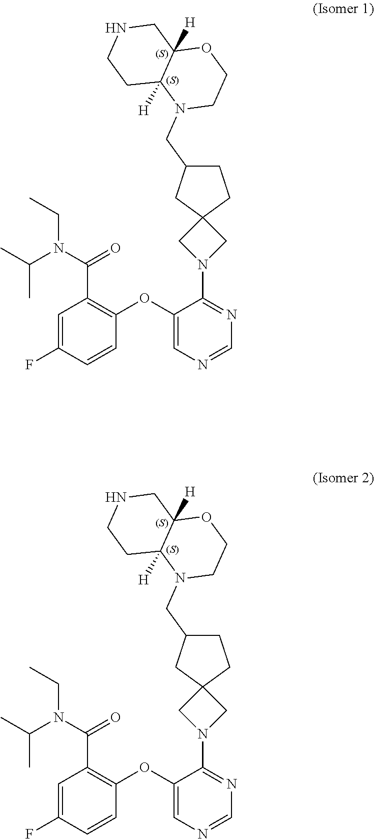

To a stirred solution of the diastereomeric mixture of tert-butyl (4aS,8aS)-1-((2(5-(2-(ethyl(isopropyl)carbamoyl)-4-fluorophenoxy)pyrimidin-4-yl)-2-azaspiro[3.4]octan-6-yl)methyl)octahydro-6H-pyrido[3,4-b][1,4]oxazine-6-carboxylate (180 mg, 0.270 mmol) in DCM (2 mL), TFA (0.021 mL, 0.270 mmol) was added at 0° C. The reaction was stirred at RT for 1 h, and monitored by TLC (10% MeOH in DCM) and LCMS. After completion, the reaction was concentrated under reduced pressure to obtain the crude diastereomeric mixture of N-ethyl-5-fluoro-N-isopropyl-2-((4-(6-(((4aS,8aS)-octahydro-1H-pyrido[3,4-b][1,4]oxazin-1-yl)methyl)-2-azaspiro[3.4]octan-2-yl)pyrimidin-5-yl)oxy)benzamide (180 mg, 37.9% yield). This material was used without further purification. LCMS (Method D): Rt=2.90-2.96 min, m/z=567.3 [M+H]⁺, 58.19%.

This diastereomeric mixture (180 mg) was purified by prep HPLC (Method C) to obtain both isomers.

Isomer 1: (D1)-N-Ethyl-5-fluoro-N-isopropyl-2-((4-(6-(((4aS,8aS)-octahydro-1H-pyrido[3,4-b][1,4]oxazin-1-yl)methyl)-2-azaspiro[3.4]octan-2-yl)pyrimidin-5-yl)oxy)benzamide (4.67 mg, 3.03% yield) as a solid. ¹H NMR (400 MHz, DMSO-d₆): δ 8.31-8.24 (m, 1H), 7.79-7.65 (m, 1H), 7.34-7.18 (m, 2H), 7.08-6.96 (m, 1H), 4.10-3.87 (m, 4H), 3.80-3.67 (m, 2H), 3.58-3.48 (m, 1H), 3.43-3.38 (m, 1H), 3.26-3.12 (m, 2H), 3.06-2.97 (m, 1H), 2.94-2.76 (m, 4H), 2.46-2.39 (m, 1H), 2.32-2.24 (m, 1H), 2.17-2.04 (m, 2H), 1.99-1.85 (m, 3H), 1.83-1.63 (m, 4H), 1.45-1.33 (m, 1H), 1.31-1.26 (m, 1H), 1.20-0.94 (m, 9H), one proton merged with solvent peaks; LCMS (Method D): Rt=1.64 min, m/z=567.3 [M+H]⁺; HPLC (Method D): Rt=1.96 min, 99.45%; Chiral SFC (Method AG): 4.64 min, 96.49%.

Isomer 2: (D2)-N-Ethyl-5-fluoro-N-isopropyl-2-((4-(6-(((4aS,8aS)-octahydro-1H-pyrido[3,4-b][1,4]oxazin-1-yl)methyl)-2-azaspiro[3.4]octan-2-yl)pyrimidin-5-yl)oxy)benzamide (4.98 mg, 3.21% yield) as a solid. ¹H NMR (400 MHz, DMSO-d₆): δ 8.33-8.20 (m, 1H), 7.79-7.62 (m, 1H), 7.33-7.21 (m, 2H), 7.09-6.96 (m, 1H), 4.12-3.86 (m, 4H), 3.80-3.65 (m, 2H), 3.57-3.48 (m, 1H), 3.43-3.37 (m, 1H), 3.26-3.11 (m, 1H), 3.03-2.93 (m, 1H), 2.87-2.76 (m, 3H), 2.41-2.36 (m, 1H), 2.28-2.20 (m, 1H), 2.17-2.03 (m, 3H), 1.96-1.85 (m, 3H), 1.82-1.67 (m, 4H), 1.59-1.49 (m, 1H), 1.22-1.16 (m, 2H), 1.15-0.93 (m, 9H), one proton merged with solvent peaks; LCMS (Method D): Rt=1.68 min, m/z=567.3 [M+H]⁺; HPLC (Method A): Rt=5.00 min, 97.34%; Chiral SFC (Method AG): 5.22 min, 100%.

### Example 46. (D1)-N-Ethyl-5-fluoro-N-isopropyl-2-((4-(6-(((4aR,8aR)-octahydro-1H-pyrido[3,4-b][1,4]oxazin-1-yl)methyl)-2-azaspiro[3.4]octan-2-yl)pyrimidin-5-yl)oxy)benzamide (Compound No. 46)

### Example 47. (D2)-N-Ethyl-5-fluoro-N-isopropyl-2-((4-(6-(((4aR,8aR)-octahydro-1H-pyrido[3,4-b][1,4]oxazin-1-yl)methyl)-2-azaspiro[3.4]octan-2-yl)pyrimidin-5-yl)oxy)benzamide (Compound No. 47)

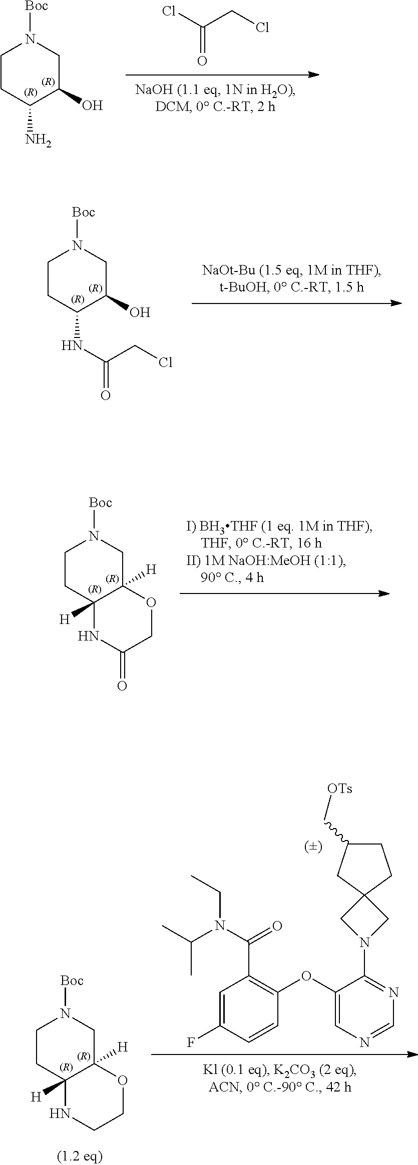

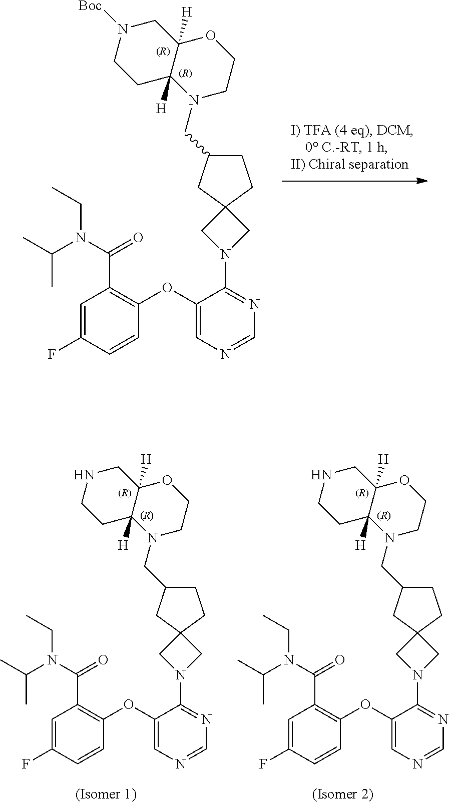

### Step 1. tert-Butyl (3R,4R)-4-(2-chloroacetamido)-3-hydroxypiperidine-1-carboxylate

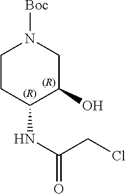

This compound was synthesized following the procedure described for the synthesis of Intermediate 7, step 1, starting with 1 equivalent of tert-butyl (3R,4R)-4-amino-3-hydroxypiperidine-1-carboxylate and 1 equivalent of 2-chloroacetyl chloride. After work-up, the organic layer was concentrated under reduced pressure to obtain crude tert-butyl (3R,4R)-4-(2-chloroacetamido)-3-hydroxypiperidine-1-carboxylate as a liquid. This material was used without further purification.

Yield: 91%; ¹H NMR (400 MHz, DMSO-d₆): δ 8.09 (br d, J=7.9 Hz, 1H), 5.06 (d, J=5.0 Hz, 1H), 4.05 (s, 2H), 3.98-3.87 (m, 1H), 3.82-3.73 (m, 1H), 3.63-3.51 (m, 1H), 3.28-3.21 (m, 1H), 2.89-2.74 (m, 1H), 2.64-2.59 (m, 1H), 1.82-1.69 (m, 1H), 1.40 (s, 9H), 1.30-1.20 (m, 1H); LCMS-ELSD (Method D): Rt=1.44 min, m/z=193.0 [M+H−100]⁺, 99.37%.

### Step 2. tert-Butyl(4aR,8aR)-2-oxooctahydro-6H-pyrido[3,4-b][1,4]oxazine-6-carboxylate

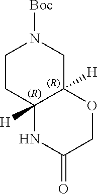

This compound was synthesized following the procedure described for the synthesis of intermediate 7, step 2, starting with 1 equivalent of tert-butyl (3R,4R)-4-(2-chloroacetamido)-3-hydroxypiperidine-1-carboxylate. After work-up, the crude was purified by silica gel flash column chromatography using EtOAc in hexane (product eluted at 30% EtOAc in hexane) to obtain ten-butyl (4aR,8aR)-2-oxooctahydro-6H-pyrido[3,4-b][1,4]oxazine-6-carboxylate as a solid.

Yield: 71.6%; ¹H NMR (400 MHz, DMSO-d₆): δ 8.25 (s, 1H), 4.10 (s, 2H), 4.08-4.02 (m, 1H), 4.00-3.88 (m, 1H), 3.26-3.16 (m, 2H), 2.83-2.62 (m, 2H), 1.90-1.79 (m, 1H), 1.40 (s, 9H), 1.32-1.19 (m, 1H); LCMS-ELSD (Method B): Rt=1.54 min, m/z=257.3 [M+H]⁺, 98.95%.

### Step 3. tert-Butyl (4aR,8aR)-octahydro-6H-pyrido[3,4-b][1,4]oxazine-6-carboxylate

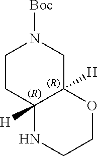

This compound was synthesized following the procedure described for the synthesis of Intermediate 7, Step 3, starting with 1 equivalent of tert-butyl (4aR,8aR)-2-oxooctahydro-6H-pyrido[3,4-b][1,4]oxazine-6-carboxylate, except that the reaction was stirred at 90° C. after the addition 1M NaOH:MeOH (1:1). After work-up, the organic layer was concentrated under reduced pressure to obtain crude tert-butyl (4aR,8aR)-octahydro-6H-pyrido[3,4-b][1,4]oxazine-6-carboxylate as a liquid. This material was used without further purification.

Yield: 51.3%; ¹H NMR (400 MHz, DMSO-d₆): δ 4.03-3.79 (m, 2H), 3.75-3.67 (m, 1H), 3.42-3.50 (m, J=3.1, 11.1 Hz, 1H), 3.39-3.26 (m, 2H), 2.90-2.80 (m, 1H), 2.80-2.66 (m, 2H), 2.38-2.28 (m, 1H), 1.63-1.53 (m, 1H), 1.39 (s, 9H), 1.27-1.10 (m, 1H), one proton merged with solvent peaks; LCMS-ELSD (Method B): Rt=1.42 min, m/z=243.3 [M+H]⁺, 97.19%.

### Step 4. tert-Butyl (4aR,8aR)-1-((2-(5-(2-(ethyl(isopropyl)carbamoyl)-4-fluorophenoxy)pyrimidin-4-yl)-2-azaspiro[3.4]octan-6-yl)methyl)octahydro-6H-pyrido[3,4-b][1,4]oxazine-6-carboxylate

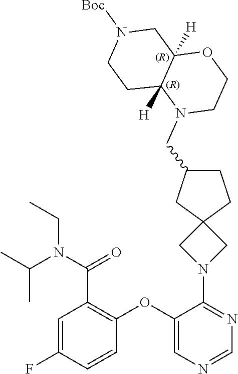

This compound was synthesized following the procedure described for the synthesis of Examples 44 and 45, Step 4, starting with 1 equivalent of (±)-(2-(5-(2-(ethyl(isopropyl)carbamoyl)-4-fluorophenoxy)pyrimidin-4-yl)-2-azaspiro[3.4]octan-6-yl)methyl 4-methylbenzenesulfonate and 1.2 equivalents of tert-butyl (4aR,8aR)-octahydro-6H-pyrido[3,4-b][1,4]oxazine-6-carboxylate, except that 0.1 equivalent of KI and 2 equivalents of K₂CO₃ were used. After work-up, the crude product was purified by silica gel flash column chromatography using EtOAc in hexane (product eluted at 60% EtOAc in hexane) to obtain the mixture of diastereomers of tert-butyl (4aR,8aR)-1-((2-(5-(2-(ethyl(isopropyl)carbamoyl)-4-fluorophenoxy)pyrimidin-4-yl)-2-azaspiro[3.4]octan-6-yl)methyl)octahydro-6H-pyrido[3,4-b][1,4]oxazine-6-carboxylate as a solid.

Yield: 43.9%; LCMS-ELSD (Method D): Rt=2.40-2.42 min, m/z=667.6 [M+H]⁺, 90.84%.

### Step 5. (D1)-N-Ethyl-5-fluoro-N-isopropyl-2-((4-(6-(((4aR,8aR)-octahydro-1H-pyrido[3,4-b][1,4]oxazin-1-yl)methyl)-2-azaspiro[3.4]octan-2-yl)pyrimidin-5-yl)oxy)benzamide and (D2)-N-ethyl-5-fluoro-N-isopropyl-2-((4-(6-(((4aR,8aR)-octahydro-1H-pyrido[3,4-b][1,4]oxazin-1-yl)methyl)-2-azaspiro[3.4]octan-2-yl)pyrimidin-5-yl)oxy)benzamide

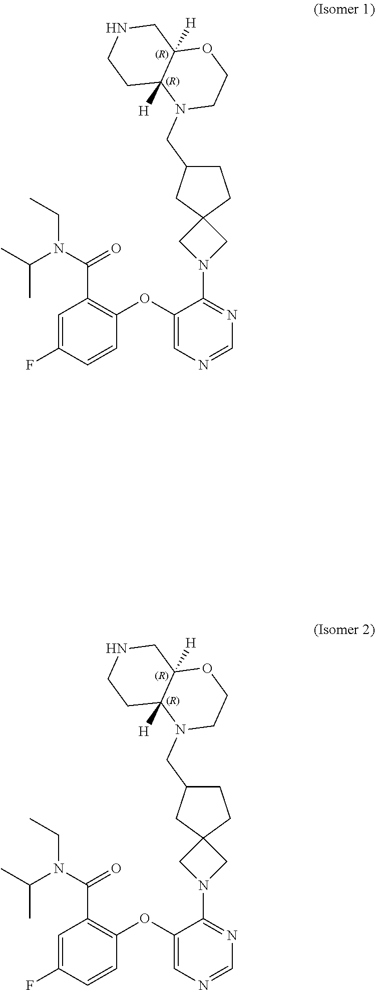

This compound was synthesized following the procedure described for the synthesis of Examples 44 and 45, Step 5, starting with 1 equivalent of the diastereomeric mixture of tert-butyl (4aR,8aR)-1-((2-(5-(2-(ethyl(isopropyl)carbamoyl)-4-fluorophenoxy)pyrimidin-4-yl)-2-azaspiro[3.4]octan-6-yl)methyl)octahydro-6H-pyrido[3,4-b][1,4]oxazine-6-carboxylate, except that 4 equivalents of TFA was used. After completion, the reaction was concentrated under reduced pressure to obtain the crude diastereomeric mixture of N-ethyl-5-fluoro-N-isopropyl-2-((4-(6-(((4aR,8aR)-octahydro-1H-pyrido[3,4-b][1,4]oxazin-1-yl)methyl)-2-azaspiro[3.4]octan-2-yl)pyrimidin-5-yl)oxy)benzamide (62% yield) as a solid. This material was used without further purification. LCMS (Method D): Rt=1.75-1.77 min, m/z=567.6 [M+H]⁺, 31.5%.

The diastereomeric mixture (200 mg) was purified by prep HPLC (Method C) to obtain both isomers.

Isomer 1: (D1)-N-Ethyl-5-fluoro-N-isopropyl-2-((4-(6-(((4aR,8aR)-octahydro-1H-pyrido[3,4-b][1,4]oxazin-1-yl)methyl)-2-azaspiro[3.4]octan-2-yl)pyrimidin-5-yl)oxy)benzamide (3.22 mg, 3.13% yield) as a solid. ¹H NMR (400 MHz, DMSO-d₆): δ 8.31-8.25 (m, 1H), 7.80-7.66 (m, 1H), 7.33-7.20 (m, 2H), 7.08-6.97 (m, 1H), 4.13-3.89 (m, 4H), 3.79-3.68 (m, 2H), 3.57-3.48 (m, 1H), 3.45-3.38 (m, 1H), 3.26-3.09 (m, 2H), 3.04-2.95 (m, 1H), 2.92-2.75 (m, 3H), 2.43-2.36 (m, 1H), 2.30-2.23 (m, 1H), 2.17-2.05 (m, 2H), 1.99-1.85 (m, 3H), 1.82-1.63 (m, 4H), 1.44-1.34 (m, 1H), 1.32-1.24 (m, 2H), 1.16-0.96 (m, 9H), one proton merged with solvent peaks; LCMS (Method D): Rt=1.60 min, m/z=567.5 [M+H]⁺; HPLC (Method A): Rt=4.84 min, 99.37%; Chiral SFC (Method W): 2.56 min, 100%.

Isomer 2: (D2)-N-Ethyl-5-fluoro-N-isopropyl-2-((4-(6-(((4aR,8aR)-octahydro-1H-pyrido[3,4-b][1,4]oxazin-1-yl)methyl)-2-azaspiro[3.4]octan-2-yl)pyrimidin-5-yl)oxy)benzamide (2.5 mg, 2.35% yield) as a solid. ¹H NMR (400 MHz, DMSO-d₆): δ 8.35-8.22 (m, 1H), 7.79-7.61 (m, 1H), 7.35-7.21 (m, 2H), 7.09-6.96 (m, 11H), 4.10-3.88 (m, 4H), 3.80-3.69 (m, 2H), 3.57-3.47 (m, 2H), 3.45-3.39 (m, 1H), 3.26-3.11 (m, 2H), 3.04-2.94 (m, 1H), 2.88-2.77 (m, 3H), 2.44-2.35 (m, 1H), 2.30-2.21 (m, 1H), 2.17-2.04 ((m, 2H), 2.00-1.85 (m, 3H), 1.83-1.68 (m, 4H), 1.59-1.49 (m, 1H), 1.29-1.21 (m, 11H), 1.17-0.90 (in, 9H), one proton merged with solvent peaks; LCMS (Method D): Rt=1.75 min, m/z=567.3 [M+H]⁺; HPLC (Method A): Rt=4.95 min, 96.72%. Chiral SFC (Method W): 3.01 min, 99.37%.

### Example 48. N-Ethyl-5-fluoro-N-isopropyl-2-((4-(6-(((4aS,8aS)-octahydro-4H-pyrido[4,3-b][1,4]oxazin-4-yl)methyl)-2-azaspiro[3.4]octan-2-yl)pyrimidin-S-yl)oxy)benzamide (Compound No. 48)

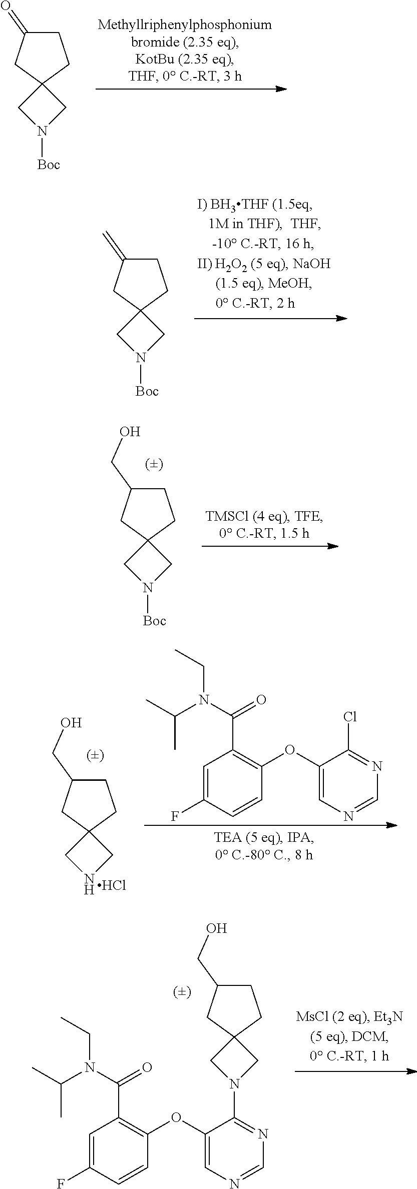

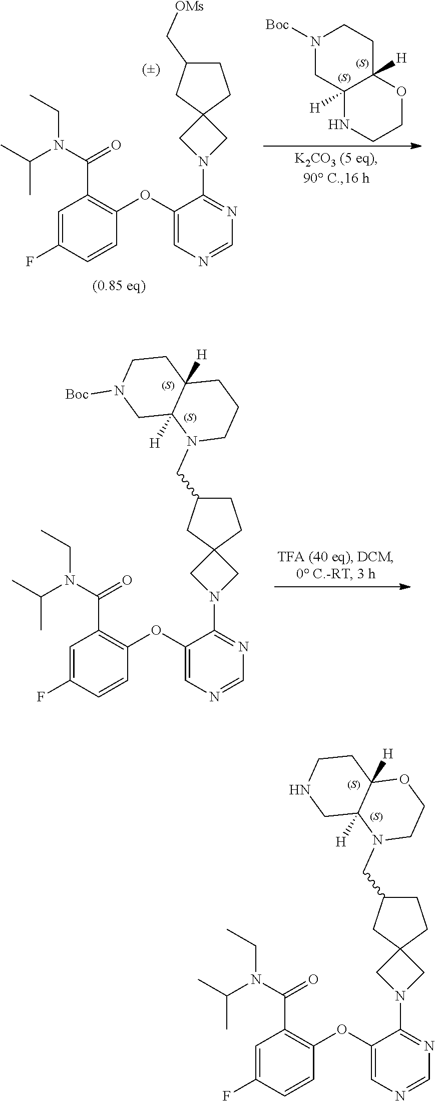

### Step 1. tert-Butyl 6-methylene-2-azaspiro[3.4]octane-2-carboxylate

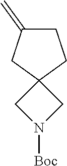 [SMILES: C=C1CCC2(C1)CN(C(=O)OC(C)(C)C)C2]

To a stirred solution of methyltriphenylphosphonium bromide (3.17 g, 8.87 mmol) in dry THF (20 mL), KOtBu (8.87 mL, 8.87 mmol) was added at 0° C. under a nitrogen atmosphere. The reaction was stirred at the same temperature for 1 h. To this reaction mixture, a solution of tert-butyl 6-oxo-2-azaspiro[3.4]octane-2-carboxylate (0.85 g, 3.77 mmol) in THF (2 mL) was slowly added at 0° C. The reaction was stirred at RT for 3 h, and monitored by TLC (20% EtOAc in hexane). After completion, the reaction was quenched with brine solution (100 mL) and extracted with EtOAc (2×150 mL). The combined organic layer was dried over anhydrous sodium sulfate and filtered, and the filtrate was concentrated under reduced pressure. The crude product was purified by silica gel flash column chromatography using EtOAc in hexane (product eluted at 5% EtOAc in hexane) to obtain tert-butyl 6-methylene-2-azaspiro[3.4]octane-2-carboxylate (0.49 g. 58.2% yield) as a liquid. ¹H NMR (400 MHz, DMSO-d₆): δ 4.93-4.87 (m, 2H), 3.81-3.74 (m, 4H), 2.49 (br s, 2H), 2.38-2.32 (m, 2H), 1.89 (t, J=7.6 Hz, 2H), 1.46 (s, 9H).

### Step 2. (i)-tert-Butyl 6-(hydroxymethyl)-2-azaspiro[3.4]octane-2-carboxylate

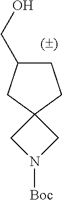 [SMILES: CC(C)(C)OC(=O)N1CC2(CCC(CO)C2)C1]

To a stirred solution of tert-butyl 6-methylene-2-azaspiro[3.4]octane-2-carboxylate (0.49 g, 2.194 mmol) in dry THF (15 mL), BH₃·THF (3.29 mL, 3.29 mmol, IM in THF) was added at −10° C. under a nitrogen atmosphere. The reaction was stirred at the same temperature for 5 min, then at RT for 16 h. MeOH (10 mL) was added at 0° C. and the reaction was stirred for 15 min at the same temperature. To this reaction mixture, 3N NaOH (1.097 mL, 3.29 mmol) and H₂O₂ (1.244 g, 10.97 mmol, 30% in water) were added at 0° C. The reaction was stirred at RT for 2 h, and monitored by TLC (50% EtOAc in hexane). After completion, the reaction was quenched with brine solution (10 mL) and extracted with EtOAc (2×25 mL). The combined organic layer was dried over anhydrous sodium sulfate and filtered, and the filtrate was concentrated under reduced pressure. The crude was purified by silica gel flash column chromatography using EtOAc in hexane (product eluted at 50% EtOAc in hexane) to obtain (±)-tert-butyl 6-(hydroxymethyl)-2-azaspiro[3.4]octane-2-carboxylate (0.4 g, 76% yield) as a gummy liquid. ¹H NMR (400 MHz, DMSO-d₆): δ 4.48 (t, J=5.2 Hz, 1H), 3.73-3.59 (m, 4H), 3.24 (t, J=5.6 Hz, 2H), 2.03 (quin, J=7.2 Hz, 1H), 1.89-1.80 (m, 1H), 1.76-1.59 (m, 3H), 1.51-1.41 (m, 1H), 1.37 (s, 9H), 1.34-1.23 (m, 1H).

### Step 3. (±)-(2-Azaspiro[3.4]octan-6-yl)methanol hydrochloride

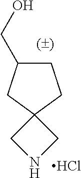

To a stirred solution of (±)-tert-butyl 6-(hydroxymethyl)-2-azaspiro[3.4]octane-2-carboxylate (0.4 g, 1.657 mmol) in TFE (6 mL), TMSCl (0.847 mL, 6.63 mmol) was added at 0° C. The reaction was stirred at RT for 1.5 h, and monitored by TLC (50% EtOAc in hexane). After completion, the reaction was concentrated under reduced pressure to obtain crude (±)-(2-azaspiro[3.4]octan-6-yl)methanol hydrochloride (0.29 g, 98% yield) as a gummy liquid. This material was used without further purification. ¹H NMR (400 MHz, DMSO-d₆): δ 8.82 (s. 2H), 4.53 (br t, J=4.5 Hz, 1H), 3.84-3.70 (m, 4H), 3.29-3.20 (m, 2H), 2.07-1.91 (m, 2H), 1.88-1.75 (m, 2H), 1.69-1.57 (m, 1H), 1.56-1.47 (m, 1H), 1.35-1.22 (m, 1H).

### Step 4. (±)-N-Ethyl-5-fluoro-2-((4-(6-(hydroxymethyl)-2-azaspiro[3.4]octan-2-yl)pyrimidin-5-yl)oxy)-N-isopropylbenzamide

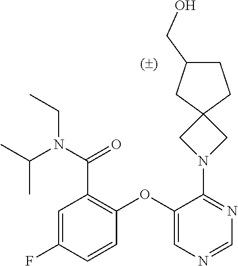 [SMILES: CCN(C(=O)c1cc(F)ccc1Oc1cncnc1N1CC2(CCC(CO)C2)C1)C(C)C]

This compound was synthesized following the procedure described for the synthesis of Example 30, step 3, starting with 1 equivalent of (±)-2-((4-chloropyrimidin-5-yl)oxy)-N-ethyl-5-fluoro-N-isopropylbenzamide and 1 equivalent of (2-azaspiro[3.4]octan-6-yl)methanol hydrochloride, except that 5 equivalents of TEA was used. After work-up, the crude was purified by silica gel flash column chromatography using MeOH in DCM (product eluted at 5% MeOH in DCM) to obtain (±)-N-ethyl-5-fluoro-2-((4-(6-(hydroxymethyl)-2-azaspiro[3.4]octan-2-yl)pyrimidin-5-yl)oxy)-N-isopropylbenzamide as a gummy liquid.

Yield: 59.8%; LCMS (Method D): Rt=1.61 min, m/z=443.3 [M+H]⁺, 71.98%.

### Step 5. (±)-(2-(5-(2-(Ethyl(isopropyl)carbamoyl)-4-fluorophenoxy)pyrimidin-4-yl)-2-azaspiro[3.4]octan-6-yl)methyl methanesulfonate

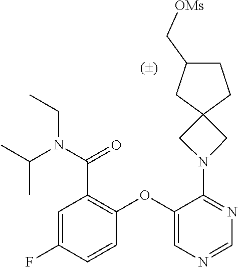 [SMILES: CCN(C(=O)c1cc(F)ccc1Oc1cncnc1N1CC2(CCC(COS(C)(=O)=O)C2)C1)C(C)C]

The reaction was performed following the same procedure in two individual batches (50 mg and 550 mg).

To a stirred solution of (±)-N-ethyl-5-fluoro-2-((4-(6-(hydroxymethyl)-2-azaspiro[3.4]octan-2-yl)pyrimidin-5-yl)oxy)-N-isopropylbenzamide (0.05 g, 0.113 mmol) in DCM (4 mL), TEA (0.079 mL, 0.565 mmol) was added at 0° C. under a nitrogen atmosphere. The reaction was stirred at the same temperature for 5 min. To this reaction mixture, MsCl (0.018 mL, 0.226 mmol) was added slowly at 0° C. The reaction was stirred at RT for 1 h, and monitored by TLC (10% MeOH in DCM). After completion, the reaction was quenched with brine solution (10 mL) and the mixture was extracted with DCM (2×25 mL). The combined organic layer was washed with brine solution (25 mL), dried over anhydrous sodium sulfate and filtered, and the filtrate was concentrated under reduced pressure to afford crude (±)-(2-(5-(2-(ethyl(isopropyl)carbamoyl)-4-fluorophenoxy)pyrimidin-4-yl)-2-azaspiro[3.4]octan-6-yl)methyl methanesulfonate (60 mg, 95% yield) as a liquid. This material was used without further purification. LCMS (Method D): Rt=1.79 min, m/z=521.1 [M+H]⁺, 93.79%.

### Step 6. tert-Butyl (4aS,8aS)-4-((2-(5-(2-(ethyl(isopropyl)carbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate

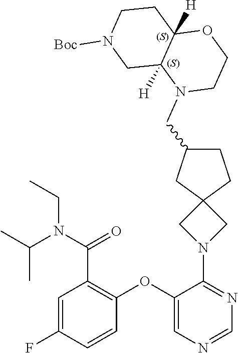

To a stirred solution of tert-butyl (4aS,8aS)-hexahydro-2H-pyrido[4,3-b][1,4]oxazine-6(5H)-carboxylate (0.18 g, 0,743 mmol) in ACN (10 mL), K₂CO₃ (0.513 g, 3.71 mmol) and (2-(5-(2-(ethyl(isopropyl)carbamoyl)-4-fluorophenoxy)pyrimidin-4-yl)-2-azaspiro[3.4]octan-6-yl)methyl methanesulfonate (0.349 g, 0.631 mmol) were added at RT under a nitrogen atmosphere. The reaction was stirred at RT for 10 min, then at 90° C. for 16 h, and monitored by TLC (10% MeOII in DCM). After completion, the reaction was quenched with brine solution (10 mL) and the mixture was extracted with EtOAc (2×20 mL). The combined organic layer was washed with brine solution (10 mL), dried over anhydrous sodium sulfate and filtered, and the filtrate was concentrated under reduced pressure to afford the crude diastereomeric mixture of tert-butyl (4aS,8aS)-4-((2-(5-(2-(ethyl(isopropyl)carbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate (450 mg, 95% yield). This material was used without further purification. LCMS (Method D): Rt=2.21 min, m/z=667.3 [M+H]⁺, 62.56%.

### Step 7. N-Ethyl-S-fluoro-V-isopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide

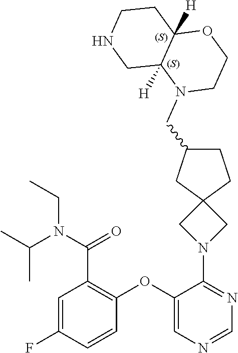

This compound was synthesized following the procedure described for the synthesis of Example 32, step 4, starting with 1 equivalent of the crude diastereomeric mixture of tert-butyl (4aS,8aS)-4-((2-(5-(2-(ethyl(isopropyl)carbamoyl)-4-fluorophenoxy)pyrimidin-4-yl)-2-azaspiro[3.4]octan-6-yl)methyl)hexahydro-2H-pyrido[4,3-b][1,4]oxazine-6(5H)-carboxylate, except that 40 equivalents of TFA was used. After completion, the reaction was concentrated under reduced pressure, and the crude was purified by prep HPLC (Method 1) to obtain the diastereomeric mixture of N-ethyl-5-fluoro-N-isopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide (0.1 g, 28.9% yield). ¹H NMR (400 MHz, DMSO-d₆): δ 8.33-8.23 (m, 1H), 7.79-7.65 (m, 1H), 7.33-7.20 (m, 2H), 7.10-6.96 (m, 1H), 4.12-3.88 (m, 4H), 3.81-3.68 (m, 2H), 3.58-3.47 (m, 1H), 3.45-3.37 (m, 1H), 3.26-3.10 (m, 2H), 3.06-2.96 (m, 1H), 2.90-2.72 (m, 2H), 2.47-2.37 (m, 2H), 2.19-1.87 (m, 6H), 1.84-1.60 (m, 5H), 1.43-1.24 (m, 2H), 1.22-0.95 (m, 9H), one proton merged with solvent peaks; LCMS (Method D): Rt=1.71 min, m/z 567.2 [M+H]⁺; HPLC (Method E): Rt=2.99 min, 98.61%; Chiral SFC (Method X): Peak-1: 11.08 min, 48.47%, Peak-2: 11.75 min, 51.53%.

### Example 49. (D1)-N-Ethyl-5-fluoro-N-isopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide (Compound No. 49)

### Example 50. (D2)-N-Ethyl-5-fluoro-N-isopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide (Compound No. 50)

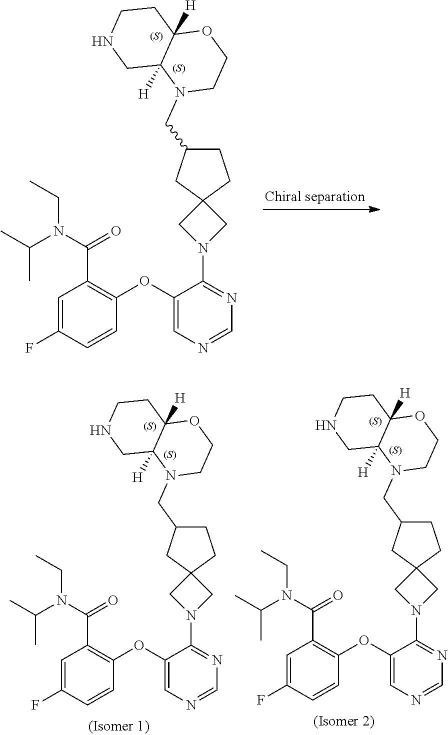

The diastereomeric mixture of N-ethyl-5-fluoro-N-isopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide (84 mg, 0.148 mmol) was purified by Chiral prep SFC (Method H) to obtain both isomers. Both isomers were found to be impure. Therefore peak-1 was re-purified by prep HPLC (Method J), and peak 2 was re-purified by prep HPLC (Method K) to obtain pure isomers.

Isomer 1: (D1)-N-Ethyl-5-fluoro-N-isopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide (20 mg, 23.75% yield) as a solid. ¹H NMR (400 MHz, DMSO-d₆): δ 8.33-8.24 (m, 1H), 7.79-7.65 (m, 1H), 7.36-7.21 (m, 2H), 7.10-6.95 (m, 1H), 4.08-3.88 (m, 4H), 3.82-3.68 (m, 2H), 3.59-3.48 (m, 1H), 3.44-3.36 (m, 2H), 3.26-3.10 (m, 3H), 3.09-2.99 (m, 1H), 2.93-2.86 (m, 1H), 2.83-2.75 (m, 1H), 2.57-2.55 (m, 11H), 2.46-2.40 (m, 1H), 2.19-2.00 (m, 3H), 1.97-1.86 (m, 2H), 1.84-1.69 (m, 4H), 1.68-1.62 (m, 1H), 1.55-1.45 (m, 1H), 1.41-1.27 (m, 1H), 1.22-0.95 (m, 9H); LCMS (Method B): Rt=1.73 min, m/z=567.4 [M+H]⁺; HPLC (Method A): Rt=4.81 min, 99.72%; Chiral SFC (Method Y): 15.4 min, 100%.

Isomer 2: (D2)-N-Ethyl-5-fluoro-N-isopropyl-2-((4-(6-(((4aS,8aS)-octahydro-4H-pyrido[4,3-b][1,4]oxazin-4-yl)methyl)-2-azaspiro[3.4]octan-2-yl)pyrimidin-5-yl)oxy)benzamide (30 mg, 35.5% yield) as a solid. ¹H NMR (400 MHz, DMSO-d₆): δ 8.29-8.24 (m, 1H), 7.77-7.66 (m, 1H), 7.33-7.21 (m, 2H), 7.08-6.98 (m, 1H), 4.08-3.88 (m, 4H), 3.78-3.69 (m, 2H), 3.58-3.49 (m, 1H), 3.45-3.39 (m, 1H), 3.26-3.19 (m, 2H), 3.16-3.02 (m, 2H), 2.95-2.88 (m, 1H), 2.83-2.74 (m, 1H), 2.57-2.55 (m, 1H), 2.47-2.44 (m, 1H), 2.20-2.04 (m, 3H), 2.00-1.91 (m, 2H), 1.86-1.62 (m, 5H), 1.44-1.32 (m, 2H), 1.30-1.23 (m, 1H), 1.24-1.17 (m, 9H); LCMS (Method B): Rt=1.71 min, m/z=567.4 [M+H]⁺; HPLC (Method A): Rt=4.72 min, 99.52%; Chiral SFC (Method Y): 17.55 min, 100%.

### Example 51. (D1)-N-Ethyl-5-fluoro-2-((4-(6-(((4aR,7aR)-hexahydropyrrolo[3,4-b][1,4]oxazin-4(4aH)-yl)methy)-2-azaspiro[3.4]octan-2-yl)pyrimidin-5-yl)oxy)-N-isopropylbenzamide (Compound No. 51)

### Example 52. (D2)-N-Ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (Compound No. 52)

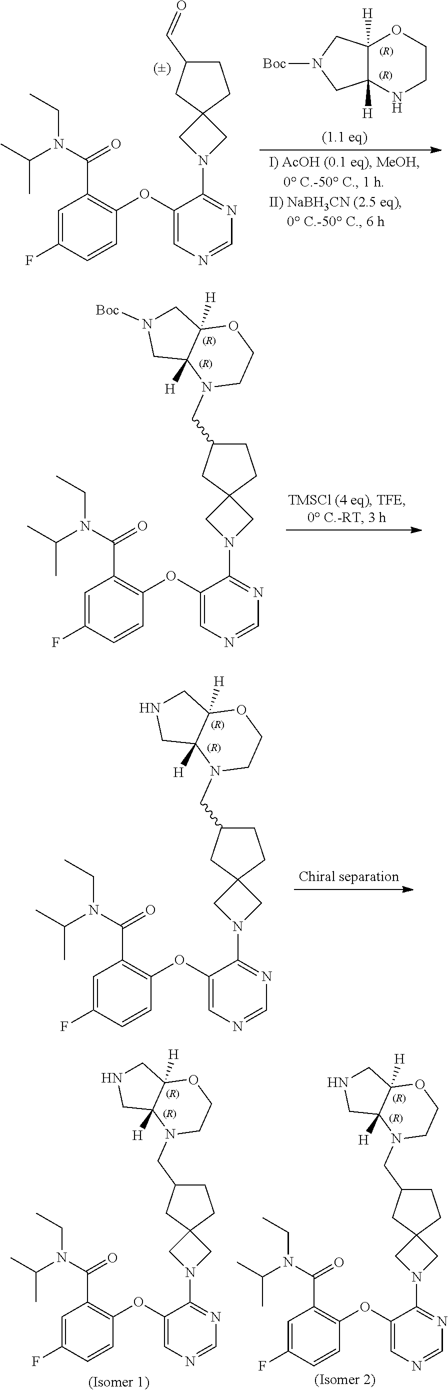

### Step 1. tert-Butyl (4aR,7aR)-4-((2-(5-(2-(ethyl(isopropyl)carbamoyl)-4-fluorophenoxy)pyrimidin-4-yl)-2-azaspiro[3.4]octan-6-yl)methyl)hexahydropyrrolo[3,4-b][1,4]oxazine-6(2H)-carboxylate

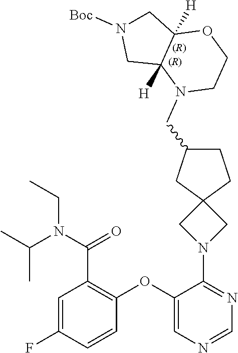

In a dried, 25 mL round bottom flask under a nitrogen atmosphere, (±)-ethyl-5-fluoro-2-((4-(6-formyl-2-azaspiro[3.4]octan-2-yl)pyrimidin-5-yl)oxy)-N-isopropylbenzamide (180 mg, 0.409 mmol) and tert-butyl (4aR,7aR)-hexahydropyrrolo[3,4-b][1,4]oxazine-6(2H)-carboxylate (121 mg, 0.531 mmol) were dissolved in MeOH (5 mL). To this reaction mixture, acetic acid (2.339 μL, 0.041 mmol) was added at 0° C., and the reaction was stirred at 50° C. for 1 h. NaBH₃CN (64.2 mg, 1.02 mmol) was then added at 0° C. The reaction was stirred at 50° C. for 6 h, and monitored by TLC (10% MeOH in DCM). After completion, the reaction was diluted with water (20 mL) and the mixture was extracted with 10% MeOH in DCM (3×25 mL). The combined organic layer was dried over anhydrous sodium sulfate and filtered, and the filtrate was concentrated under reduced pressure. The crude product was purified by silica gel flash column using MeOH in DCM (product eluted at 10% MeOH in DCM) to obtain the diastereomeric mixture of tert-butyl (4aR,7aR)-4-((2-(5-(2-(ethyl(isopropyl)carbamoyl)-4-fluorophenoxy)pyrimidin-4-yl)-2-azaspiro[3.4]octan-6-yl)methyl)hexahydropyrrolo[3,4-b][1,4]oxazine-6(2H)-carboxylate (120 mg, 29.5% yield) as a gummy solid. LCMS (Method D): Rt=2.19 min, m/z=653.2 [M+H]⁺, 65.50%.

### Step 2. N-Ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide

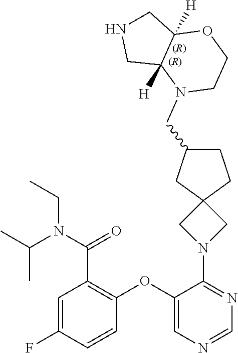

This compound was synthesized following the procedure described for the synthesis of Example 34, step 4, starting with 1 equivalent of tert-butyl (4aR,7aR)-4-((2-(5-(2-(ethyl(isopropyl)carbamoyl)-4-fluorophenoxy)pyrimidin-4-yl)-2-azaspiro[3.4]octan-6-yl)methyl)hexahydropyrrolo[3,4-b][1,4]oxazine-6(2H)-carboxylate. After completion, the reaction was concentrated under reduced pressure. The crude was purified by prep HPLC (Method N) to obtain the diastereomeric mixture of N-ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide as a solid.

Yield: 54.1%; ¹H NMR (400 MHz, DMSO-d₆): δ 8.32-8.24 (m, 1H), 7.77-7.66 (m, 1H), 7.33-7.19 (m, 2H), 7.08-6.97 (m, 1H), 4.09-3.89 (m, 4H), 3.87-3.81 (m, 1H), 3.78-3.70 (m, 1H), 3.61-3.50 (m, 1H), 3.45-3.39 (m, 1H), 3.25-3.11 (m, 1H), 3.05-2.89 (m, 2H), 2.86-2.77 (m, 1H), 2.63-2.55 (m, 1H), 2.47-2.42 (m, 1H), 2.26-1.90 (m, 7H), 1.84-1.68 (m, 3H), 1.56-1.34 (m, 1H), 1.19-0.95 (m, 9H), two protons merged with solvent peaks; LCMS (Method D): Rt=1.62 min, m/z=553.2 [M+H]⁺; HPLC (Method A): Peak 1: Rt=4.36 min, 49.66%, Peak 2: Rt=4.45 min, 50.24%; Chiral SFC (Method AA): Peak 1: Rt=6.02 min, 47.89%, Peak 2: Rt=6.93 min, 51.31%.

### Step 3. (D1)-N-Ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)(oxy)-N-isopropylbenzamide and (D2)-N-ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide

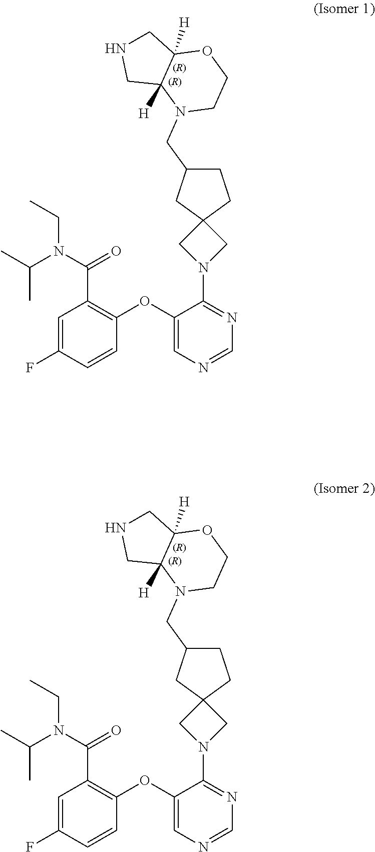

The diastereomeric mixture of N-ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (50 mg, 0.090 mmol) was purified by Chiral prep SFC (Method E) to obtain both isomers.

Isomer 1: (D1)-N-Ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (21 mg, 41.9% yield) as a solid. ¹H NMR (400 MHz, DMSO-d₆): δ 8.29-8.25 (m, 11H), 7.77-7.65 (m, 1H), 7.33-7.20 (m, 2H), 7.08-6.95 (m, 1H), 4.10-3.82 (m, 5H), 3.79-3.70 (m, 1H), 3.64-3.51 (m, 2H), 3.47-3.40 (m, 1H), 3.22-3.09 (m, 11H), 3.05-2.91 (m, 2H), 2.88-2.80 (m, 1H), 2.64-2.54 ((m, 1H), 2.46-2.41 (m, 1H), 2.24-2.04 (m, 4H), 2.02-1.89 (m, 2H), 1.86-1.67 (m, 4H), 1.57-1.47 (m, 1H), 1.23-0.96 (m, 9H), one proton merged with solvent peaks; LCMS (Method D): Rt=1.58 min, m/z=553.2 [M+H]⁺; HPLC (Method A): Rt=4.47 min, 99.88%; Chiral SFC (Method AB): Rt=10.9 min, 100%.

Isomer 2: (D2)-N-Ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)oxy)-V-isopropylbenzamide (21 mg, 41.9% yield) as a solid. ¹H NMR (400 MHz, DMSO-d₆): δ 8.31-8.24 (m, 11H), 7.76-7.66 (m, 1H), 7.34-7.20 (m, 2H), 7.10-6.95 (m, 1H), 4.09-3.89 (m, 4H), 3.86-3.81 (m, 1H), 3.79-3.69 (m, 1H), 3.64-3.49 (m, 2H), 3.45-3.40 (m, 1H), 3.22-3.10 (m, 1H), 3.06-2.91 (m, 2H), 2.88-2.78 (m, 1H), 2.63-2.54 (m, 2H), 2.46-2.44 (m, 11H), 2.26-2.17 (m, 1H), 2.13-1.90 (m, 5H), 1.82-1.65 (m, 3H), 1.46-1.35 (m, 1H), 1.21-0.96 (m, 9H), one proton merged with solvent peaks; LCMS (Method D): Rt=1.55 min, m/z=553.2 [M+H]⁺; HPLC (Method A): Rt=4.37 min, 99.79%; Chiral SFC (Method AB): Rt=14.03 min, 100%.

### Example 53. N-Ethyl-5-fluoro-2-((4-(6-(((4aS,7aS)-hexahydropyrrolo[3,4-b][1,4]oxazin-4(4aH)-yl)methyl)-2-azaspiro[3,4]octan-2-yl)pyrimidin-5-yl)oxy)-N-isopropylbenzamide (Compound No. 53)

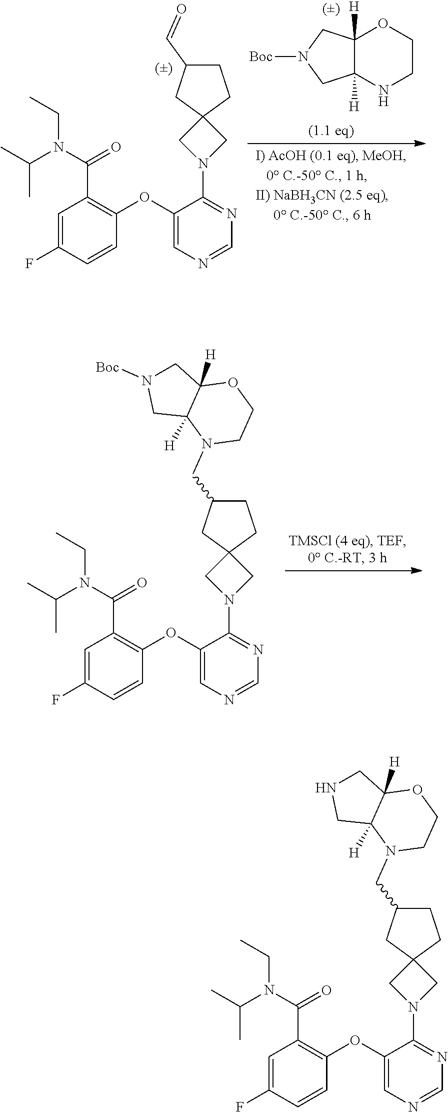 [SMILES: CCN(C(=O)c1cc(F)ccc1Oc1cncnc1N1CC2(CCC(C=O)C2)C1)C(C)C ; CC(C)(C)OC(=O)N1C[C@@H]2NCCO[C@H]2C1 ; CCN(C(=O)c1cc(F)ccc1Oc1cncnc1N1CC2(CCC(CN3CCO[C@H]4CN(C(=O)OC(C)(C)C)C[C@@H]43)C2)C1)C(C)C ; CCN(C(=O)c1cc(F)ccc1Oc1cncnc1N1CC2(CCC(CN3CCO[C@H]4CNC[C@@H]43)C2)C1)C(C)C]

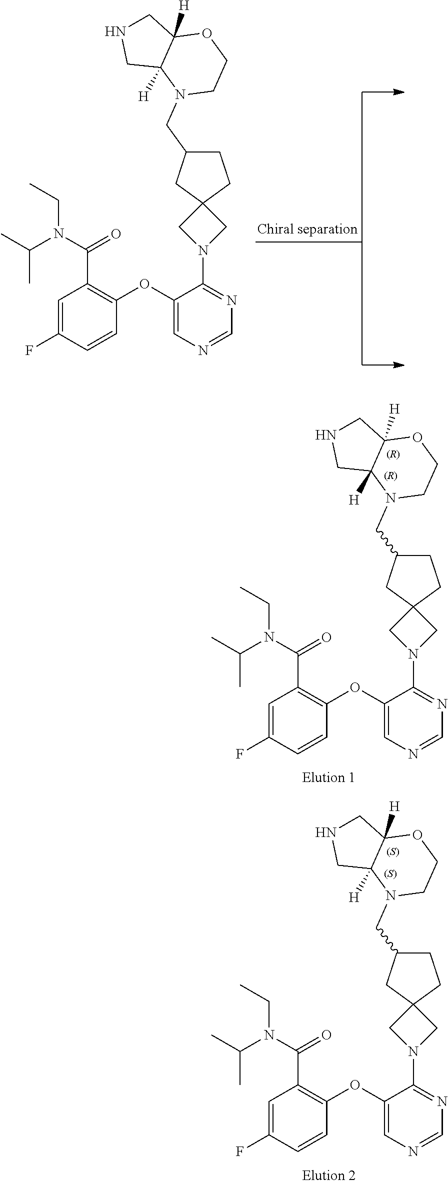

### Step 1. tert-Butyl (trans)-4-((2-(5-(2-(ethyl(isopropyl)carbamoyl)-4-fluorophenoxy)pyrimidin-4-yl)-2-azaspiro[3.4]octan-6-yl)methyl)hexahydropyrrolo[3,4-b][1,4]oxazine-6(2H)-carboxylate

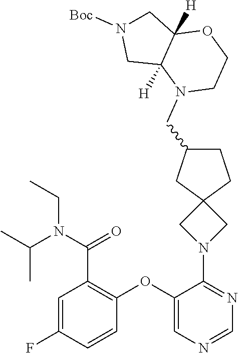 [SMILES: CCN(C(=O)c1cc(F)ccc1Oc1cncnc1N1CC2(CCC(CN3CCO[C@H]4CN(C(=O)OC(C)(C)C)C[C@@H]43)C2)C1)C(C)C]

In a dried, 50 mL round bottom flask under a nitrogen atmosphere, (±)-N-ethyl-5-fluoro-2-((4-(6-formyl-2-azaspiro[3.4]octan-2-yl)pyrimidin-5-yl)oxy)-N-isopropylbenzamide (700 mg, 1.589 mmol) and (±)-tert-butyl (trans)-hexahydropyrrolo[3,4-b][1,4]oxazine-6(2H)-carboxylate (399 mg, 1.748 mmol) were dissolved in MeOH (5 mL). To this reaction mixture, AcOH (9.54 mg, 0.159 mmol) was added at RT and the reaction was stirred at 50° C. for 1 h. NaBH₃CN (250 mg, 3.97 mmol) was then added at 0° C. The reaction was stirred at 50° C. for 6 h, and monitored by LCMS. After completion, the reaction was diluted with water (50 mL) and extracted with 10% MeOH in DCM (3×50 mL). The combined organic layer was washed with brine solution (100 mL), dried over anhydrous sodium sulfate and filtered, and the filtrate was concentrated under reduced pressure. The crude product was purified by silica gel flash column using MeOH in DCM (product eluted at 10% MeOH in DCM) to obtain the diastereomeric mixture of tert-butyl (trans)-4-((2-(5-(2-(ethyl(isopropyl)carbamoyl)-4-fluorophenoxy)pyrimidin-4-yl)-2-azaspiro[3.4]octan-6-yl)methyl)hexahydropyrrolo[3,4-b][1,4]oxazine-6(2H)-carboxylate (430 mg, 38.0% yield) as a solid. LCMS (Method D): Rt=2.18 min, m/z 653.3 [M+H]⁺, 79.55%.

### Step 2. N-Ethyl-5-fluoro-2-((4-(6-(((trans)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide

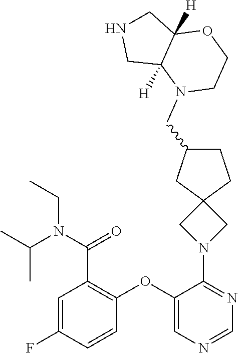 [SMILES: CCN(C(=O)c1cc(F)ccc1Oc1cncnc1N1CC2(CCC(CN3CCO[C@H]4CNC[C@@H]43)C2)C1)C(C)C]

In a dried, 25 mL round bottom flask under a nitrogen atmosphere, the diastereomeric mixture of tert-butyl (trans)-4-((2-(5-(2-(ethyl(isopropyl)carbamoyl)-4-fluorophenoxy)pyrimidin-4-yl)-2-azaspiro[3.4]octan-6-yl)methyl)hexahydropyrrolo[3,4-b][1,4]oxazine-6(2H)-carboxylate (430 mg, 0.659 mmol) was dissolved in TFE (8 mL). To this solution, TMSCl (0.337 mL, 2.63 mmol) was added at 0° C. and the reaction was stirred at RT for 3 h. The reaction progress was monitored by LCMS. After completion, the reaction was concentrated under reduced pressure. The crude was purified by prep HPLC (Method G) to afford the diastereomeric mixture of N-ethyl-5-fluoro-2-((4-(6-(((trans)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (145 mg, 38.8% yield) as a solid. ¹H NMR (400 MHz, DMSO-d₆): δ 8.37-8.20 (m, 1H), 7.82-7.62 (m, 1H), 7.40-7.21 (m, 2H), 7.10-6.98 ((m, 1H), 4.16-3.89 (m, 4H), 3.87-3.82 (m, 1H), 3.79-3.72 (m, 1H), 3.66-3.46 (m, 3H), 3.22-3.14 (m, 1H), 3.05-2.89 (m, 2H), 2.86-2.81 (m, 1H), 2.61-2.57 (m, 1H), 2.45-2.39 (m, 1H), 2.26-1.89 (m, 6H), 1.85-1.66 (m, 3H), 1.57-1.37 (m, 1H′), 1.32-1.24 (m, 1H), 1.21-0.95 (m, 9H), one proton merged with solvent peaks; LCMS (Method D): Rt=1.60 min, m/z=553.1 [M+H]⁺; HPLC (Method A): Peak 1: Rt=4.52 min, 48.57/6, Peak 2: Rt=4.62 min, 48.75%; Chiral SFC (Method Z): Peak 1: Rt=3.12 min, 50.66%, Peak 2: Rt=4.15 min, 49.34%.

### Step 3. N-Ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (Elution 1) and N-ethyl-5-fluoro-2-((4-(6-(((4aS,7aS)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (Elution 2)

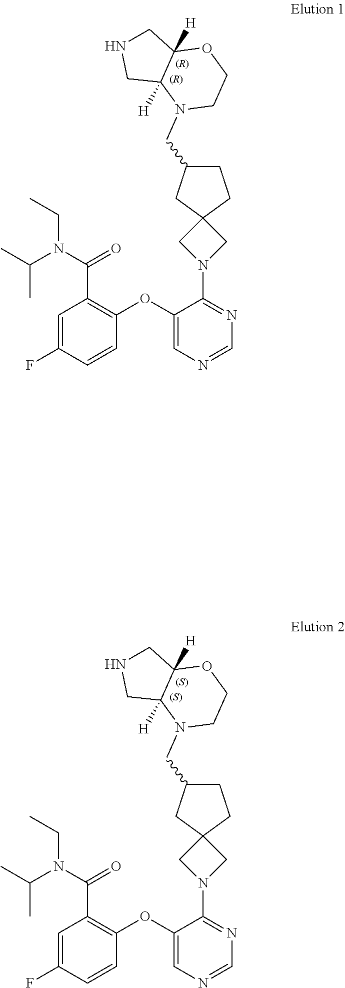

The diastereomeric mixture of N-ethyl-5-fluoro-2-((4-(6-(((trans)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (140 mg, 0.253 mmol) was purified by Chiral prep SFC (Method 1) to obtain two fractions. Each elution contains a pair of diastereomers.

Elution 1: N-Ethyl-5-fluoro-2-((4-(6-(((4aR,7aR)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (60 mg, 42.6% yield) as a solid. ¹H NMR (400 MHz, DMSO-d₆): δ 8.32-8.23 (m, 1H), 7.77-7.65 (m, 1H), 7.33-7.20 (m, 2H), 7.09-6.97 (m, 1H), 4.08-3.81 (m, 5H), 3.78-3.69 (m, 1H), 3.64-3.51 (m, 2H), 3.46-3.40 (m, 1H), 3.23-3.10 (m, 1H), 3.04-2.97 (m, 1H), 2.95-2.90 (m, 1H), 2.88-2.78 (m, 1H), 2.63-2.56 (m, 1H), 2.47-2.40 (m, 1H), 2.28-1.88 (m, 6H), 1.84-1.67 (m, 3H), 1.56-1.35 (m, 1H), 1.32-1.25 (m, 1H), 1.21-0.94 (m, 9H), one proton merged with solvent peaks; LCMS (Method D): Rt=1.49 min, m/z=553.2 [M+H]⁺; HPLC (Method A): Peak 1: Rt=4.43 min, 52.93%, Peak 2: Rt=4.52 min, 46.37%; Chiral SFC (Method AB): Peak 1: Rt=3.94 min, 46.48%, Peak 2: Rt=4.73 min, 53.52%.

Elution 2: N-Ethyl-5-fluoro-2-((4-(6-(((4aS,7aS)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (55 mg, 38.8% yield) as a solid. ¹H NMR (400 MHz, DMSO-d₄): δ 8.32-8.21 (m, 1H), 7.78-7.64 (m, 1H), 7.35-7.20 (m, 2H), 7.08-6.96 (m, 1H), 4.11-3.83 (m, 5H), 3.79-3.69 (m, 1H), 3.66-3.50 (m, 2H), 3.23-3.05 (m, 3H), 3.03-2.97 (m, 1H), 2.92-2.79 (m, 1H), 2.66-2.61 (m, 1H), 2.27-1.89 (m, 6H), 1.86-1.67 (m, 3H), 1.57-1.35 (m, 1H), 1.30-1.25 (m, 1H), 1.22-0.96 (m, 9H), two protons merged with solvent peaks; LCMS (Method D): Rt=1.50 min, m/z=553.2 [M+H]⁺; HPLC (Method A): Peak 1: Rt=4.42 min, 44.51%, Peak 2: Rt=4.51 min, 54.35%; Chiral SFC (Method AG): Peak 1: Rt=5.00 min, 43.95%, Peak 2: Rt=5.39 min, 56.05%.

### Example 54. (D1)-N-Ethyl-5-fluoro-2-((4-(6-(((4aS,7aS)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (Compound No. 54)

### Example 55. (D2)-N-Ethyl-5-fluoro-2-((4-(6-(((4aS,7aS)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (Compound No. 55)

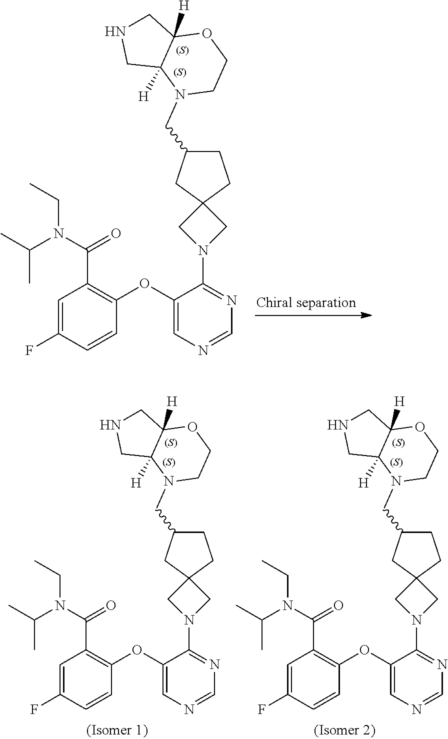

The diastereomeric mixture of N-ethyl-5-fluoro-2-((4-(6-(((4aS,7aS)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (Elution 2) (50 mg) was purified by Chiral prep SFC (Method B) to obtain both isomers. Subsequently, isomer 2 was re-purified by Chiral prep SFC (Method C).

Isomer 1: (D1)-N-Ethyl-5-fluoro-2-((4-(6-(((4aS,7aS)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (12 mg, 23.42% yield) as a solid. ¹H NMR (400 MHz, DMSO-d₆): δ 8.37-8.24 (m, 1H), 7.80-7.68 (m, 1H), 7.34-7.21 (m, 2H), 7.09-6.95 (m, 1H), 4.10-3.84 (m, 5H), 3.79-3.71 (m, 1H), 3.63-3.52 (m, 1H), 3.48-3.44 (m, 1H), 3.23-3.13 (m, 2H), 2.92-2.82 (m, 1H), 2.80-2.73 (m, 1H), 2.66-2.61 (m, 1H), 2.58-2.54 (m, 1H), 2.47-2.42 (m, 1H), 2.29-2.19 (m, 1H), 2.16-2.04 (m, 3H), 2.01-1.91 (m, 2H), 1.84-1.67 (m, 3H), 1.46-1.35 (m, 1H), 1.32-1.26 (m, 1H), 1.22-0.96 (m, 9H), one proton merged with solvent peaks; LCMS (Method D): Rt=1.64 min, m/z=553.2 [M+H]⁺; HPLC (Method A): Rt=4.45 min, 97.60%; Chiral SFC (Method AC): Rt=6.35 min, 100%.

Isomer 2: (D2)-N-Ethyl-5-fluoro-2-((4-(6-(((4aS,7aS)-hexahydropyrrolo[3,4-b][1,4]oxazin-4(4aH)-yl)methyl)-2-azaspiro[3.4]octan-2-yl)pyrimidin-5-yl)oxy)-N-isopropylbenzamide (30 mg, 45.9% yield) as a solid. ¹H NMR (400 MHz, DMSO-d₆): δ 8.31-8.15 (m, 1H), 7.82-7.66 (m, 1H), 7.33-7.20 (m, 2H), 7.07-6.96 (m, 1H), 4.07-3.85 (m, 5H), 3.81-3.70 (m, 1H), 3.62-3.53 (m, 1H), 3.47-3.39 (m, 2H), 3.25-3.12 (m, 3H), 2.94-2.77 (m, 2H), 2.66-2.57 (m, 1H), 2.27-2.07 (m, 3H), 2.01-1.91 (m, 2H), 1.86-1.69 (m, 3H), 1.59-1.42 (m, 1H), 1.32-1.23 (m, 2H), 1.21-0.96 (m, 9H), two protons merged with solvent peaks; LCMS (Method B): Rt=1.15 min, m/z=553.4 [M+H]⁺; HPLC (Method E): Rt=2.99 min, 95.22%; Chiral SFC (Method AC): Rt=7.56 min, 100%.

### Example 56. 5-Fluoro-N,N-diisopropyl-2-((4-(7-(((4aS,8aS)-octahydro-4H-pyrido[4,3-b][1,4]oxazin-4-yl)methyl)-5-oxa-2-azaspiro[3,4]octan-2-yl)pyrimidin-5-yl)oxy)benzamide (Compound No. 56)

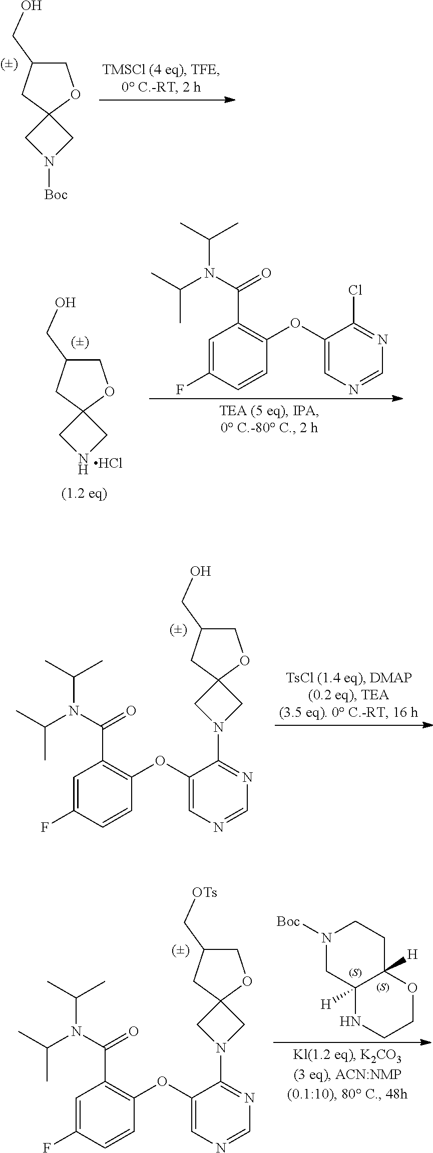

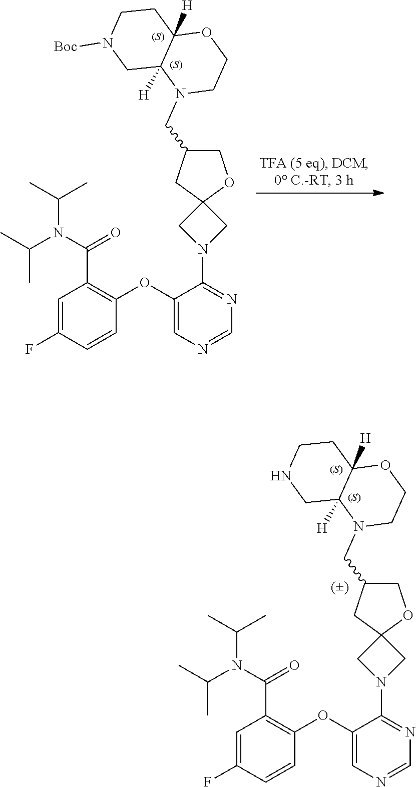

### Step1. (±)-(5)-Oxa-azaspiro[3,4]octan-7-yl)methanol hydrochloride

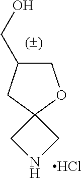

This compound was synthesized following the procedure described for the synthesis of Intermediate 15, Step 1, starting with 1 equivalent of (±)-tert-butyl 7-(hydroxymethyl)-5-oxa-2-azaspiro[3.4]octane-2-carboxylate, except that 4 equivalents of TMSCl was used. After completion, the reaction was concentrated under reduced pressure to obtain crude (±)-(5-oxa-2-azaspiro[3.4]octan-7-yl)methanol hydrochloride (220 mg, 99% yield). This material was used without further purification. ¹H NMR (400 MHz, DMSO-d₆): δ 8.72 (br s, 2H), 6.06 (t, J=6.4 Hz, 1H), 4.74 (t, J=5.2 Hz, 1H), 4.05-3.81 (m, 5H), 3.57 (dd, J=8.4, 6.0 Hz, 1H), 3.31-3.25 (m, 1H), 2.41-2.32 (m, 1H), 2.27-2.18 (m, 1H), 1.92-1.87 (m, 1H).

### Step 2. (±)-5-Fluoro-2-((4-(7-(hydroxymethyl)-5-oxa-2-azaspiro[3.4]octan-2-yl)pyrimidin-5-yl)oxy)-N,N-diisopropylbenzamide

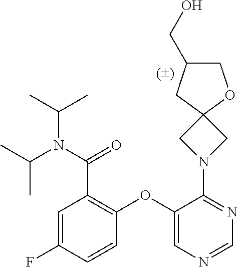 [SMILES: CC(C)N(C(=O)c1cc(F)ccc1Oc1cncnc1N1CC2(CC(CO)CO2)C1)C(C)C]

This compound was synthesized following the procedure described for the synthesis of Intermediate 11, starting with 1 equivalent of 2-((4-chloropyrimidin-5-yl)oxy)-S-fluoro-N,N-diisopropylbenzamide and 1.2 equivalents of (±)-(5-oxa-2-azaspiro[3.4]octan-7-yl)methanol, except that 5 equivalents of TEA was used. After completion, the reaction was concentrated under reduced pressure, water was added, and the mixture was extracted with EtOAc. The combined organic layer was washed with water, dried over anhydrous sodium sulfate and filtered, and the filtrate was concentrated under reduced pressure. The crude product was purified by silica gel flash column chromatography using EtOAc in hexane (product eluted at 100% EtOAc in hexane) to afford (±)-5-fluoro-2-((4-(7-(hydroxymethyl)-5-oxa-2-azaspiro[3.4]octan-2-yl)pyrimidin-5-yl)oxy)-N,N-diisopropylbenzamide as a gummy liquid.

Yield: 88%; ¹H NMR (400 MHz, DMSO-d₆): δ 8.30-8.29 (m, 1H), 7.79-7.76 (in, 1H), 7.26-7.19 (m, 2H), 7.05-6.99 (m, 1H), 4.69 (t, J=5.1 Hz, 1H), 4.28-4.18 (m, 1H), 4.15-4.11 (m, 2H), 4.06-4.03 (m, 1H), 3.81 (t, J=7.9 Hz, 1H), 3.73-3.64 (m, 1H), 3.59-3.49 (m, 2H), 3.38-3.35 (m, 1H), 3.30-3.22 (m, 1H), 2.42-2.32 (m, 1H), 2.21-2.12 (m, 1H), 1.87-1.78 (m, 1H), 1.44 (d, J=6.6 Hz, 3H), 1.34 (d, J=6.5 Hz, 3H), 1.09 (d, J=6.6 Hz, 3H), 1.04-0.97 (m, 3H); LCMS (Method D): Rt=1.54 min, m/z=459.4 [M+H]⁺, 99.32%.

### Step 3. (±)-(2-(5-(2-(Diisopropylcarbamoyl)-4-fluorophenoxy)pyrimidin-4-yl)-5-oxa-2-azaspiro[3.4]octan-7-yl)methyl 4-methylbenzenesulfonate

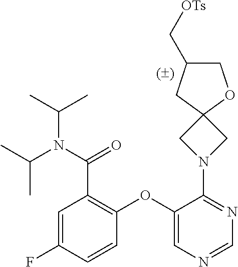 [SMILES: Cc1ccc(S(=O)(=O)OCC2COC3(C2)CN(c2ncncc2Oc2ccc(F)cc2C(=O)N(C(C)C)C(C)C)C3)cc1]

This compound was synthesized following the procedure described for the synthesis of Intermediate 18, Step 2, starting with 1 equivalent of (+)-5-fluoro-2-((4-(7-(hydroxymethyl)-5-oxa-2-azaspiro[3.4]octan-2-yl)pyrimidin-5-yl)oxy)-N,N-diisopropylbenzamide and 1.4 equivalents of p-TsCl, except that 0.2 equivalent of DMAP and 3.5 equivalents of TEA were used. After work-up, the crude was purified by silica gel flash column chromatography using EtOAc in hexane (product eluted at 50% EtOAc in hexane) to afford (+)-2-(5-(2-(diisopropylcarbamoyl)-4-fluorophenoxy)pyrimidin-4-yl)-5-oxa-2-azaspiro[3.4]octan-7-yl)methyl 4-methylbenzenesulfonate as a gummy liquid.

Yield: 94%; LCMS (Method D): Rt=2.06 min, m/z=613.2 [M+H]⁺, 97.19%.

### Step 4. tert-Butyl (4aS,8aS)-4-((2-(5-(2-(diisopropylcarbamoyl)-4-fluorophenoxy)pyrimidin-4-yl)-5-oxa-2-azaspiro[3.4]octan-7-yl)methyl)hexahydro-2H-pyrido[4,3-b][1,4]oxazine-6(5H)-carboxylate

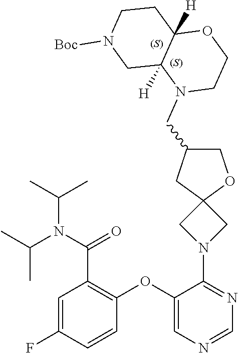

To a stirred solution of (±)-(2-(5-(2-(diisopropylcarbamoyl)-4-fluorophenoxy)pyrimidin-4-yl)-5-oxa-2-azaspiro[3.4]octan-7-yl)methyl 4-methylbenzenesulfonate (0.260 g, 0.424 mmol) in 0.1:10 ACN:NMP (0.1 mL:10 mL), tert-butyl (4aS,8aS)-hexahydro-2H-pyrido[4,3-b][1,4]oxazine-6(5H)-carboxylate (0.134 g, 0.552 mmol), K₂CO₃ (0.176 g, 1.273 mmol), and KI (0.085 g, 0.509 mmol) were added at 0° C. under a nitrogen atmosphere. The reaction was stirred at 80° C. for 48 h, and monitored by TLC (10% MeOH in DCM). After completion, the reaction was quenched with water (20 mL) and the mixture was extracted with EtOAc (2×20 mL). The combined organic layer was dried over anhydrous sodium sulfate and filtered, and the filtrate was concentrated under reduced pressure. The crude was purified by silica gel flash column chromatography using EtOAc in hexane (product eluted at 100% EtOAc) to afford the diastereomeric mixture of tert-butyl (4aS,8aS)-4-((2-(5-(2-(diisopropylcarbamoyl)-4-fluorophenoxy)pyrimidin-4-yl)-5-oxa-2-azaspiro[3.4]octan-7-yl)methyl)hexahydro-2H-pyrido[4,3-b][1,4]oxazine-6(5H)-carboxylate (0.110 g, 25.8% yield) as a gummy liquid. LCMS (Method D): Rt=2.15 min, m/z=683.6 [M+H]⁺, 68.27%.

### Step 5. 5-Fluoro-N,N-diisopropyl-2-((4-(7-(((4aS,8aS)-octahydro-4H-pyrido[4,3-b][1.4]oxazin-4-yl)methyl)-5-oxa-2-azaspiro[3.4]octan-2-yl)pyrimidin-5-yl)oxy)benzamide

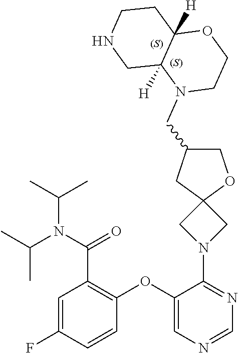

To a solution of the diastereomeric mixture of tert-butyl (4aS,8aS)-4-((2-(5-(2-(diisopropylcarbamoyl)-4-fluorophenoxy)pyrimidin-4-yl)-5-oxa-2-azaspiro[3.4]octan-7-yl)methyl)hexahydro-2H-pyrido[4,3-b][1,4]oxazine-6(5H)-carboxylate (0.1 g, 0.146 mmol) in DCM (2 mL), TFA (0.056 mL, 0.732 mmol) was added at 0° C. The reaction was stirred at RT for 3 h, and monitored by TLC (100% EtOAc). After completion, water (20 mL) was added and the mixture was extracted with EtOAc (100 mL). The combined organic layer was dried over anhydrous sodium sulfate and filtered, and the filtrate was concentrated under reduced pressure. The crude was purified by prep HPLC (Method D) to obtain the diastereomeric mixture of 5-fluoro-N,N-diisopropyl-2-((4-(7-(((4aS,8aS)-octahydro-4H-pyrido[4,3-b][1,4]oxazin-4-yl)methyl)-5-oxa-2-azaspiro[3.4]octan-2-yl)pyrimidin-5-yl)oxy)benzamide (0.015 g, 17.23% yield) as a solid. ¹H NMR (400 MHz, DMSO-d₆): δ 8.32-8.28 (m, 1H), 7.81-7.76 (m, 1H), 7.27-7.19 (m, 2H), 7.04-6.97 (m, 1H), 4.30-3.97 (m, 4H), 3.90-3.79 (m, 1H), 3.76-3.66 (m, 2H), 3.59-3.44 (m, 3H), 3.25-3.16 (m, 2H), 3.08-2.97 (m, 1H), 2.93-2.74 (m, 2H), 2.61-2.55 (m, 1H), 2.47-2.39 (m, 2H), 2.27-2.09 (m, 2H), 2.07-1.90 (m, 2H), 1.85-1.74 (m, 1H), 1.67-1.61 (m, 1H), 1.44 (d, J=6.7 Hz, 3H), 1.38-1.24 (m, 4H), 1.09 (d, J=6.6 Hz, 3H), 1.04-0.99 (m, 3H), one proton merged with solvent peaks; LCMS (Method D): Rt=1.50 min, m/z=583.5 [M+H]⁺; HPLC (Method D): Rt=2.67 min, 98.86%; Chiral SFC (Method AD): Peak 1: Rt=4.39 min, 53.66%, Peak 2: Rt=4.80 min, 46.34%.

### Example 57. (D1)-5-Fluoro-N,N-Diisopropyl-2-((4-(7-(((4aS,8aS)-octahydro-4H-pyrido[4,3-b][1,4]oxazin-4-yl)methyl)-5-oxa-2-azaspiro[3.4]octan-2-yl)pyrimidin-5-yl)oxy)benzamide (Compound No. 57)

### Example 58. (D2)-5-Fluoro-N,N-disopropyl-2-((4-(7-(((4aS,8aS)-octahydro-4H-pyrido[4,3-b][1,4]oxazin-4-yl)methyl)-5-oxa-2-azaspiro[3,4]octan-2-yl)pyrimidin-5-yl)oxy)benzamide (Compound No. 58)

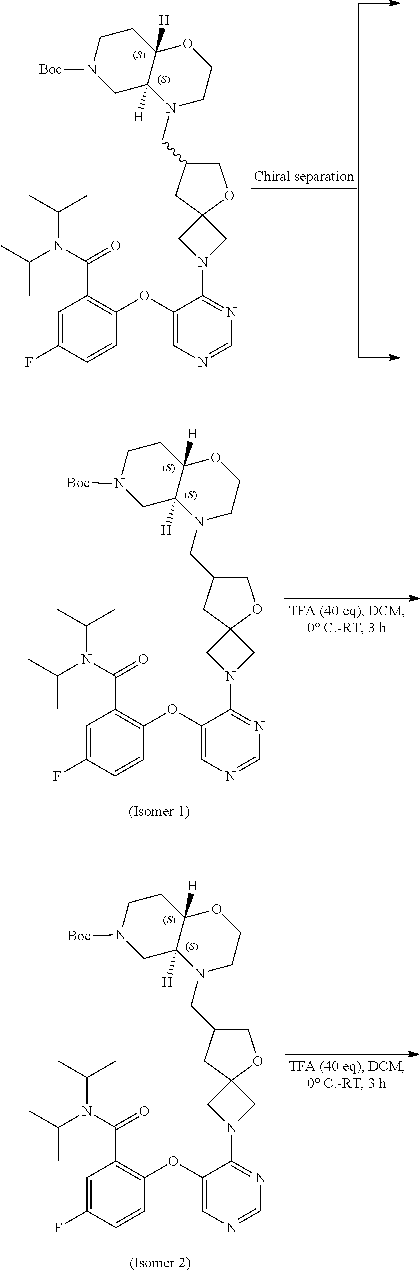

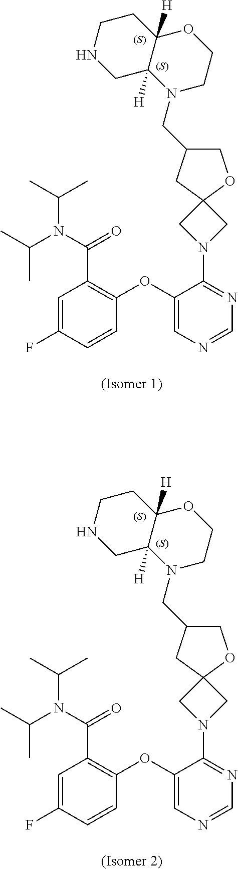

### Step 1. (D1)-tert-Butyl (4a,8aS)-4-((2-(5-(2-(diisopropylcarbamoyl)-4-fluorophenoxy)pyrimidin-4-yl)-5-oxa-2-azaspiro[3.4]octan-7-yl)methyl)hexahydro-2H-pyrido[4,3-b][1.4]oxazine-6(5H)-carboxylate and (D2)-tert-butyl (4aS,8aS)-4-((2-(5-(2-(diisopropylcarbamoyl)-4-fluorophenoxy)pyrimidin-4-yl)-5-oxa-2-azaspiro[3.4]octan-7-yl)methyl)hexahydro-2H-pyrido[4,3-b][1,4]oxazine-6(5H)-carboxylate

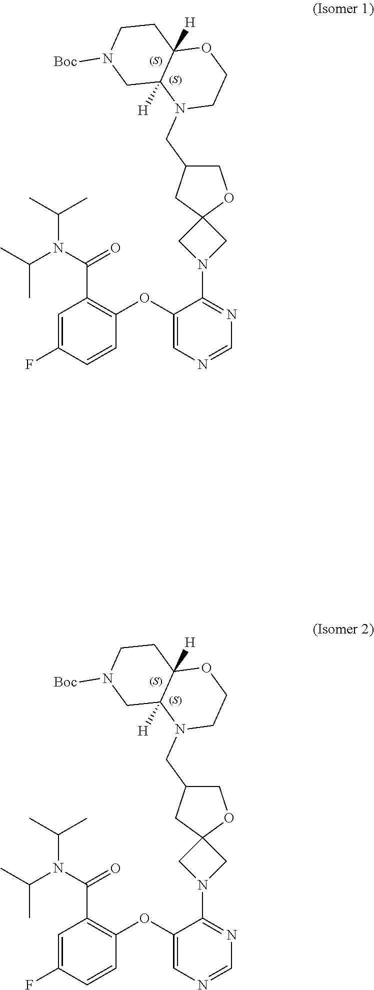

The diastereomeric mixture of 5-fluoro-N,N-diisopropyl-2-((4-(7-(((4aS,8aS)-octahydro-4H-pyrido[4,3-b][1,4]oxazin-4-yl)methyl)-5-oxa-2-azaspiro[3,4]octan-2-yl)pyrimidin-5-yl)oxy)benzamide (170 mg, 0.25 mmol) was purified by Chiral prep HPLC (Method D) to obtain both isomers.

Isomer 1: (D1)-tert-Butyl (4a,8aS)-4-((2-(5-(2-(diisopropylcarbamoyl)-4-fluorophenoxy)pyrimidin-4-yl)-5-oxa-2-azaspiro[3.4]octan-7-yl)methyl)hexahydro-2H-pyrido[4,3-b][1,4]oxazine-6(5H)-carboxylate (0.055 g, 19.35% yield) as a semi-solid. LCMS (Method B): Rt=1.99 min, m/z=683.5 [M+H]⁺, 98.90%; Chiral SFC (Method AE): Rt=8.46 min, 100%.

Isomer 2: (D2)-tert-Butyl (4aS,8aS)-4-((2-(5-(2-(diisopropylcarbamoyl)-4-fluorophenoxy)pyrimidin-4-yl)-5-oxa-2-azaspiro[3.4]octan-7-yl)methyl)hexahydro-2H-pyrido[4,3-b][1,4]oxazine-6(5H)-carboxylate (0.045 g, 15.83% yield) as a semi-solid. LCMS (Method B): Rt=1.97 min, m/z=683.5 [M+H]⁺, 98.60%; Chiral SFC (Method AE): Rt=9.48 min, 98.81%.

### Step 2. (D1)-5-Fluoro-N,N-diisopropyl-2-((4-(7-(((4aS,8aS)-octahydro-4H-pyrido[4,3-b][1,4]oxazin-4-yl)methyl)-5-oxa-2-azaspiro[3.4]octan-2-yl)pyrimidin-5-yl)oxy)benzamide

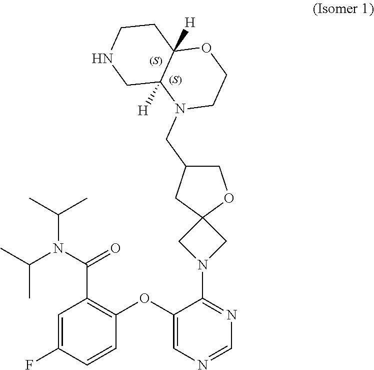

This compound was synthesized following the procedure described for the synthesis of Example 56, Step 5, starting with 1 equivalent of (D1)-tert-butyl (4aS,8aS)-4-((2-(5-(2-(diisopropylcarbamoyl)-4-fluorophenoxy)pyrimidin-4-yl)-5-oxa-2-azaspiro[3.4]octan-7-yl)methyl)hexahydro-2H-pyrido[4,3-b][1,4]oxazine-6(5H)-carboxylate, except that 40 equivalents of TFA was used. After completion, the crude was purified by reverse phase chromatography using a SiliCycle SiliaSep C18 column (40 g) on Biotage isolera one (Mobile phase A: 10 mM NH₄HCO₃ in water, B: ACN) to obtain (D1)-5-fluoro-N,N-diisopropyl-2-((4-(7-(((4aS,8aS)-octahydro-4H-pyrido[4,3-b][1,4]oxazin-4-yl)methyl)-5-oxa-2-azaspiro[3,4]octan-2-yl)pyrimidin-5-yl)oxy)benzamide as a solid.

Yield: 56.4%; ¹H NMR (400 MHz, DMSO-d₆): δ 8.30 (s, 1H), 7.78 (s, 1H), 7.27-7.19 (m, 2H), 7.03-6.95 (m, 1H), 4.33-3.97 (m, 4H), 3.82 (t, J=7.6 Hz, 1H), 3.75-3.63 (m, 2H), 3.58-3.43 (m, 3H), 3.24-3.15 (m, 1H), 3.06-2.96 (m, 1H), 2.92-2.77 (m, 2H), 2.59 (br s, 1H), 2.44 (br s, 1H), 2.26-2.19 (m, 1H), 2.17-2.08 (m, 1H), 2.06-1.95 (m, 2H), 1.83-1.71 (m, 2H), 1.68-1.59 (m, 1H), 1.44 (d, J=6.7 Hz, 3H), 1.34 (d, J=6.7 Hz, 3H), 1.31-1.22 (m, 1H), 1.09 (d, J=6.4 Hz, 3H), 1.01 (br d, J=6.3 Hz, 3H), two protons merged with solvent peaks; LCMS (Method B): Rt=1.54 min, m/z=583.4 [M+H]⁺; H PLC (Method D): Rt=2.65 min, 99.90%; Chiral SFC (Method P): Rt=4.18 min, 98.57%.

### Step 3. (D2)-S-Fluoro-N,N-diisopropyl-2-((4-(7-(((4aS,8aS)-octahydro-4H-pyrido[4,3-b][1,4]oxazin-4-yl)methyl)-5-oxa-2-azaspiro[3.4]octan-2-yl)pyrimidin-5-yl)oxy)benzamide

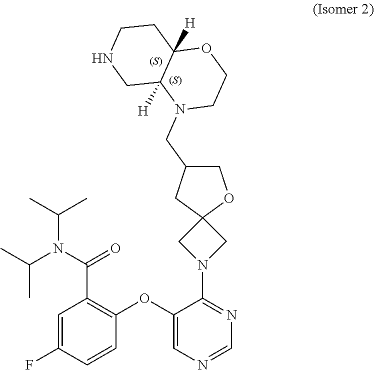

This compound was synthesized following the procedure described for the synthesis of Example 56, Step 5, starting with 1 equivalent of (D2)-tert-butyl (4aS,8aS)-4-((2-(5-(2-(diisopropylcarbamoyl)-4-fluorophenoxy)pyrimidin-4-yl)-5-oxa-2-azaspiro[3.4]octan-7-yl)methyl)hexahydro-2H-pyrido[4,3-b][1,4]oxazine-6(5H)-carboxylate, except that 40 equivalents of TFA was used. After completion, the crude was purified by reverse phase chromatography using a SiliCycle SiliaSep C18 column (40 g) on Biotage isolera one (Mobile phase A: 10 mM NH₄HCO₃ in water, B: ACN) to obtain (D2)-5-fluoro-N,N-diisopropyl-2-((4-(7-(((4aS,8aS)-octahydro-4H-pyrido[4,3-b][1,4]oxazin-4-yl)methyl)-5-oxa-2-azaspiro[3.4]octan-2-yl)pyrimidin-5-yl)oxy)benzamide as a solid.

Yield: 62.0%; ¹H NMR (400 MHz, DMSO-d₆): δ8.33-8.28 (m, 1H), 7.81-7.74 (m, 1H), 7.27-7.17 (m, 2H), 7.05-6.94 (m, 1H), 4.29-4.00 (m, 4H), 3.85 (t, J=7.6 Hz, 1H), 3.76-3.65 (m, 2H), 3.59-3.49 (m, 2H), 3.47-3.41 (m, 1H), 3.27-3.22 (m, 1H), 3.20-3.13 (m, 1H), 3.10-2.96 (m, 1H), 2.91-2.82 (m, 1H), 2.81-2.76 (m, 1H), 2.45-2.36 (m, 2H), 2.20-2.10 (m, 2H), 2.01-1.87 (m, 3H), 1.84-1.75 (m, 1H), 1.68-1.60 (m, 1H), 1.44 (d, J=6.6 Hz, 3H), 1.38-1.33 (m, 3H), 1.31-1.20 (m, 1H), 1.09 (d, J=6.5 Hz, 3H), 1.05-0.97 (m, 3H), one proton merged with solvent peaks; LCMS (Method B): Rt=1.63 min, m/z=583.4 [M+H]⁺; HPLC (Method D): Rt=2.63 min, 99.22%; Chiral SFC (Method V): Rt=4.51 min, 99.45%.

### Example 59. 5-Fluoro-N,N-diisopropyl-2-((4-(7-(((4aS,8aS)-octahydro-4H-pyrido[4,3-b][1,4]oxazin-4-yl)methyl)-6-oxa-2-azaspiro[3.4]octan-2-yl)pyrimidin-5-yl)oxy)benzamide (Compound No. 59)

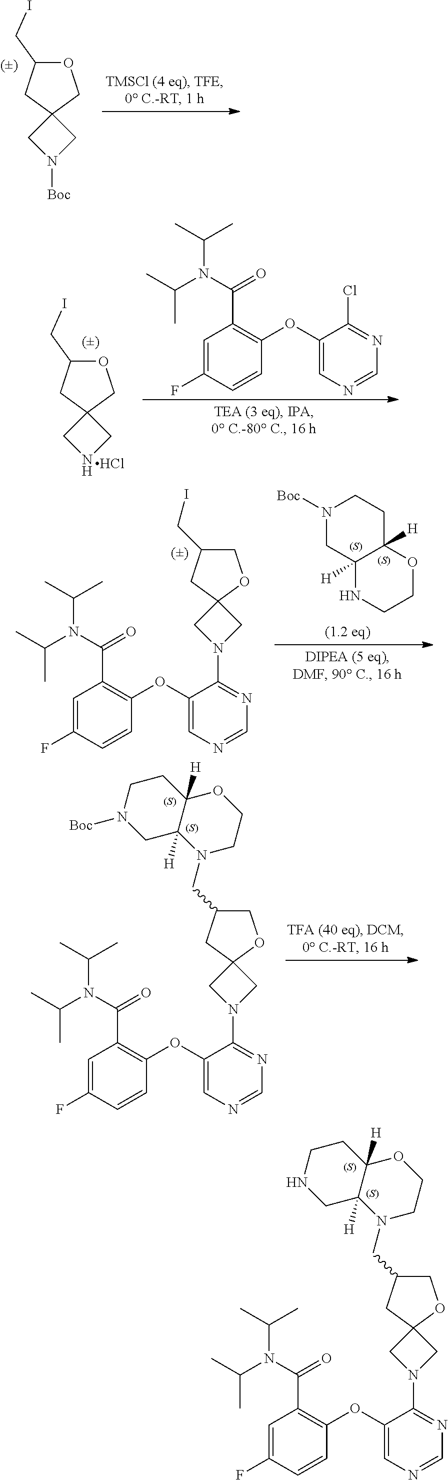

### Step 1. (±)-7-Iodomethyl)-6-oxa-2-azaspiro[3.4]octane hydrochloride

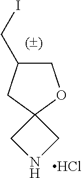

This compound was synthesized following the procedure described for the synthesis of Intermediate 15, Step 1, starting with 1 equivalent of (±)-tert-butyl 7-(iodomethyl)-6-oxa-2-azaspiro[3.4]octane-2-carboxylate, except that 4 equivalents of TMSCl was used. After completion, the reaction was concentrated under reduced pressure and re-concentrated from EtOAc to obtain crude (±)-7-(iodomethyl)-6-oxa-2-azaspiro[3.4]octane hydrochloride as a gummy liquid. This material was used without further purification.

Yield: 98%; ¹H NMR (400 MHz, DMSO-d₆): δ 9.09-8.53 (m, 2H), 4.04-3.80 (m, 61H), 3.40-3.36 (m, 1H), 3.31-3.25 (m, 1H), 2.42 (dd, J=6.8, 13.2 Hz, 1H), 1.86 (dd, J=7.6, 13.2 Hz, 1H).

### Step 2. (±)-5-Fluoro-2-((4-(7-(iodomethyl)-6-oxa-2-azaspiro[3.4]octan-2-yl)pyrimidin-5-yl)oxy)-N,N-diisopropylbenzamide

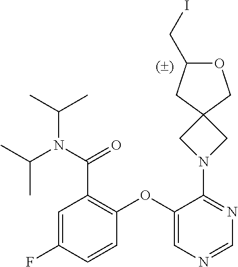 [SMILES: CC(C)N(C(=O)c1cc(F)ccc1Oc1cncnc1N1CC2(COC(CI)C2)C1)C(C)C]

This compound was synthesized following the procedure described for the synthesis of Intermediate 11, starting with 1 equivalent of (±)-5-fluoro-2-((4-(7-(iodomethyl)-6-oxa-2-azaspiro[3.4]octan-2-yl)pyrimidin-5-yl)oxy)-N,N-diisopropylbenzamide and 1 equivalent of tert-butyl (4aS,8aS)-hexahydro-2H-pyrido[4,3-b][1,4]oxazine-6(5H)-carboxylate. The reaction was stirred at 80° C. After completion, the reaction was concentrated under reduced pressure. The crude was purified by silica gel flash column chromatography using MeOH in DCM (product eluted at 5% MeOH in DCM) to obtain (i)-5-fluoro-2-((4-(7-(iodomethyl)-6-oxa-2-azaspiro[3.4]octan-2-yl)pyrimidin-5-yl)oxy)-N,N-diisopropylbenzamide as a semi-solid.

Yield: 24.7%; ¹H NMR (400 MHz, DMSO-d₆): δ8.30 (s, 1H), 7.81-7.73 (m, 1H), 7.28-7.20 (m, 2H), 7.09-7.01 (m, 1H), 4.28-4.04 (m, 4H), 4.00-3.90 (m, 2H), 3.85 (dd, J=3.9, 8.8 Hz, 1H), 3.73-3.63 (m, 1H), 3.58-3.49 (m, 1H), 3.40-3.35 (m, 1H), 3.30-3.26 (m, 1H), 2.39-2.32 ((m, 1H), 1.91-1.80 ((m, 1H), 1.44 (d, J=6.7 Hz, 3H), 1.34 (br s, 3H), 1.09 (d, J=6.6 Hz, 3H), 0.99 (br dd, J=1.2, 6.4 Hz, 3H); LCMS (Method D): Rt=2.03 min, m/z=569.4 [M+H]⁺, 89.84%.

### Step 3. tert-Butyl (4aS,8aS)-4-((2-(5-(2-(diisopropylcarbamoyl)-4-fluorophenoxy)pyrimidin-4-yl)-6-oxa-2-azaspiro[3.4]octan-7-yl)methyl)hexahyrdo-2H-pyrido[4,3-b][1,4]oxazine-6(5H)-carboxylate

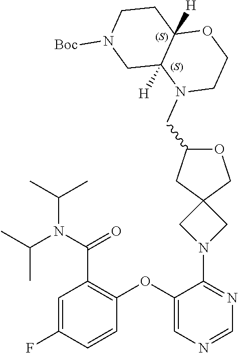

To a stirred solution of tert-butyl (4aS,8aS)-hexahydro-2H-pyrido[4,3-b][1,4]oxazine-6(5H)-carboxylate (51.2 mg, 0.211 mmol) in DMF (2.5 mL), DIPEA (0.153 mL, 0.880 mmol) was added at RT under a nitrogen atmosphere. The reaction was stirred at the same temperature for 5 min. To this reaction mixture, (±)-5-fluoro-2-((4-(7-(iodomethyl)-6-oxa-2-azaspiro[3.4]octan-2-yl)pyrimidin-5-yl)oxy)-N,N-diisopropylbenzamide (100 mg, 0.176 mmol) was added at RT, and the reaction was stirred at 90° C. for 16 h. The reaction was monitored by LCMS. After completion, the reaction was quenched with water (10 mL) and extracted with EtOAc (3×25 mL). The combined organic layer was dried over anhydrous sodium sulfate and filtered, and the filtrate was concentrated under reduced pressure. The crude was purified by silica gel flash column chromatography using EtOAc in hexane (product eluted at 40% EtOAc in hexane) to obtain the diastereomeric mixture of tert-butyl (4aS,8aS)-4-((2-(5-(2-(diisopropylcarbamoyl)-4-fluorophenoxy)pyrimidin-4-yl)-6-oxa-2-azaspiro[3.4]octan-7-yl)methyl)hexahydro-2H-pyrido[4,3-b][1,4]oxazine-6(5H)-carboxylate (15 mg, 7.32% yield) as a semi-solid. LCMS (Method D): Rt=1.98 min, m/z=683.7 [M+H]⁺, 58.65%.

### Step 4. 5-Fluoro-N,N-diisopropyl-2-((4-(7-(((4aS,8aS)-octahydro-4H-pyrido[4,3-b][1,4]oxazin-4-yl)methyl)-6-oxa-2-azaspiro[3.4]octan-2-yl)pyrimidin-5-yloxy)benzamide

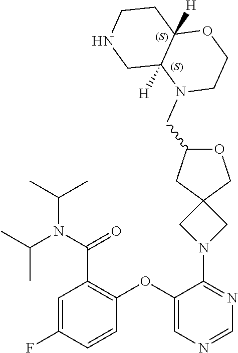

This compound was synthesized following the procedure described for the synthesis of Example 56, Step 5, starting with 1 equivalent of the diastereomeric mixture of tert-butyl (4aS,8aS)-4-((2-(5-(2-(diisopropylcarbamoyl)-4-fluorophenoxy)pyrimidin-4-yl)-6-oxa-2-azaspiro[3.4]octan-7-yl)methyl)hexahydro-2H-pyrido[4,3-b][1,4]oxazine-6(5H)-carboxylate, except that 40 equivalents of TFA was used. After completion, the reaction was concentrated under reduced pressure. The crude was purified by prep HPLC (Method L) to obtain the diastereomeric mixture of 5-fluoro-N,N-diisopropyl-2-((4-(7-(((4aS,8aS)-octahydro-4H-pyrido[4,3-b][1,4]oxazin-4-yl)methyl)-6-oxa-2-azaspiro[3.4]octan-2-yl)pyrimidin-5-yl)oxy)benzamide as a solid.

Yield: 43.7%; ¹H NMR (400 MHz, DMSO-d₆): δ 8.32-8.28 (m, 1H), 7.79-7.75 (m, 1H), 7.27-7.19 (m, 2H), 7.11-7.00 (m, 1H), 4.25-3.96 (m, 5H), 3.92-3.84 (m, 1H), 3.78-3.63 (m, 3H), 3.59-3.44 (m, 2H), 3.23-3.08 (m, 1H), 3.06-2.98 (m, 1H), 2.91-2.75 (m, 2H), 2.66-2.60 (m, 1H), 2.43-2.38 (m, 1H), 2.31-2.14 (m, 3H), 2.10-1.98 (m, 1H), 1.92-1.75 (m, 2H), 1.66-1.56 (m, 1H), 1.44 (d, J=6.7 Hz, 3H), 1.35 (d, J=6.6 Hz, 3H), 1.32-1.25 (m, 1H), 1.09 (d, J 6.6 Hz, 3H), 1.04-0.96 (m, 3H), one proton merged with solvent peaks; LCMS (Method D): Rt=1.47 min, m/z=583.8 [M+H]⁺; HPLC (Method A): Rt=4.59 min, 99.52%; Chiral SFC (Method P): Peak 1: Rt=3.26 min, 49.12%, Peak 2: Rt=3.49 min, 50.88%.

### Example 60. 5-Fluoro-N,N-diisopropyl-2-((4-(6-(((4aS,8aS)-octahydro-4H-pyrido[4,3-b][1,4]oxazin-4-yl)methyl)-S-oxa-2-azaspiro[3,4]octan-2-yl)pyrimidin-5-yl)oxy)benzamide (Compound No. 60)

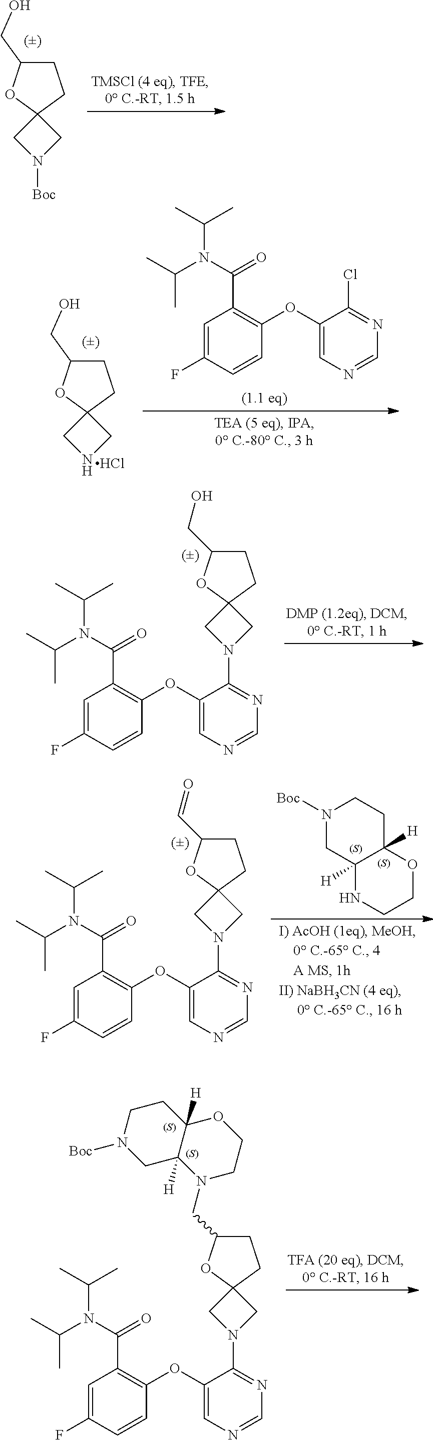

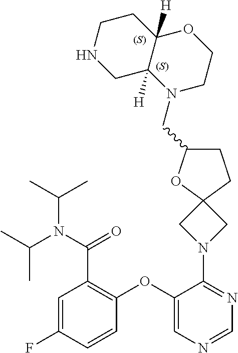

### Step 1. (±)-(5-Oxa-2-azaspiro[3.4]octan-6-yl) methanol hydrochloride

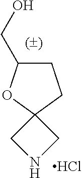

This compound was synthesized following the procedure described for the synthesis of Intermediate 15, Step 1, starting with 1 equivalent of (±)-tert-butyl 6-(hydroxymethyl)-5-oxa-2-azaspiro[3.4]octane-2-carboxylate, except that 4 equivalents of TMSCl was used. After completion, the reaction was concentrated under reduced pressure to obtain crude (±)-(5-oxa-2-azaspiro[3.4]octan-6-yl)methanol hydrochloride as a solid. This material was used without further purification.

Quantitative yield; ¹H NMR (400 MHz, DMSO-d₆): δ 9.09-8.48 (m, 2H), 4.72 (br s, 1H), 4.01-3.82 (m, 5H), 3.44-3.36 (m, 1H), 3.29-3.25 (m, 1H), 2.27-2.16 (m, 1H), 2.11-2.00 (m, 1H), 1.95-1.82 (m, 1H), 1.78-1.65 (m, 1H).

### Step 2. (±)-5-Fluoro-2-((4-(6-(hydroxymethyl)-5-oxa-2-azaspiro[3.4]octan-2-yl)pyrimidin-5-yl)oxy)-N,N-diisopropylbenzamide

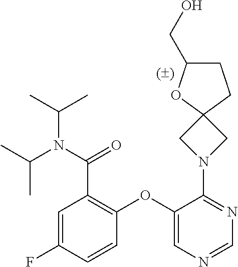 [SMILES: CC(C)N(C(=O)c1cc(F)ccc1Oc1cncnc1N1CC2(CCC(CO)O2)C1)C(C)C]

This compound was synthesized following the procedure described for the synthesis of Intermediate 11, starting with 1 equivalent of (±)-(5-oxa-2-azaspiro[3.4]octan-6-yl)methanol hydrochloride and 1.1 equivalents of 2-((4-chloropyrimidin-5-yl)oxy)-5-fluoro-N,N-diisopropylbenzamide. The reaction was stirred at 80° C. After work-up, the organic layer was concentrated under reduced pressure to obtain crude (±)-5-fluoro-2-((4-(6-(hydroxymethyl)-5-oxa-2-azaspiro[3.4]octan-2-yl)pyrimidin-5-yl)oxy)-N,N-diisopropylbenzamide as a solid. This material was used without further purification.

Yield: 92%. LCMS (Method D): Rt=1.57 min, m/z 459.1 [M+H]⁺, 74.93%.

### Step 3. (i)-5-Fluoro-2-((4-(6-formyl-5-oxa-2-azaspiro[3.4]octan-2-yl)pyrimidin-5-yl)oxy)-N,N-diisopropylbenzamide

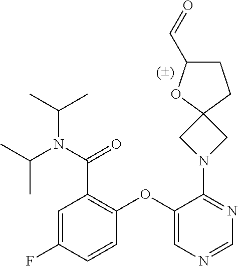 [SMILES: CC(C)N(C(=O)c1cc(F)ccc1Oc1cncnc1N1CC2(CCC(C=O)O2)C1)C(C)C]

This compound was synthesized following the procedure described for the synthesis of Example 41, Step 2, starting with 1 equivalent of (±)-5-fluoro-2-((4-(6-(hydroxymethyl)-5-oxa-2-azaspiro[3.4]octan-2-yl)pyrimidin-5-yl)oxy)-N,N-diisopropylbenzamide, except that 1.2 equivalents of DMP was used. After completion, the reaction was quenched with aqueous NaHCO₃ solution and the mixture was extracted with EtOAc. The combined organic layer was washed with brine solution, dried over anhydrous sodium sulfate and filtered, and the filtrate was concentrated under reduced pressure to afford crude (±)-5-fluoro-2-((4-(6-formyl-5-oxa-2-azaspiro[3.4]octan-2-yl)pyrimidin-5-yl)oxy)-N,N-diisopropylbenzamide as a solid. This material was used without further purification. This material was used without further purification.

Yield: 98%.

### Step 4. tert-Butyl (4aS,8aS)-4-((2-(S-(2-(diisopropylcarbamoyl)-4-fluorophenoxy)pyrimidin-4-yl)-S-oxa-2-azaspiro[3.4]octan-6-yl)methyl)hexahydro-2H-pyrrolo[4,3-b][1,4]oxazine-6(5H)-carboxylate

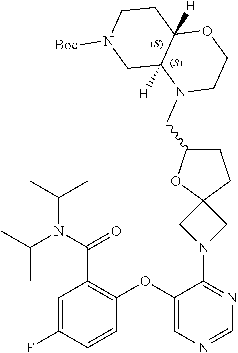

In a dried, 50 mL round bottom flask under a nitrogen atmosphere, (±)-5-fluoro-2-((4-(6-formyl-5-oxa-2-azaspiro[3.4]octan-2-yl)pyrimidin-5-yl)oxy)-N,N-diisopropylbenzamide (47.1 mg, 0.103 mmol) was dissolved in MeOH (5 mL). To this solution, tert-butyl (4aS,8aS)-hexahydro-2H-pyrido[4,3-b][1,4]oxazine-6(5H)-carboxylate (25 mg, 0.103 mmol), 4A molecular sieves (0.103 mmol), and AcOH (5.90 μL, 0.103 mmol) were added at RT. The reaction was stirred at 65° C. for 1 h. NaBH₃CN (25.9 mg, 0.413 mmol) was then added at 0° C., and the reaction was stirred at 65° C. for 16 h. The reaction progress was monitored by LCMS. The reaction was filtered through a Celite® pad, and the pad was washed with MeOH (3×20 mL), and the filtrate was concentrated under reduced pressure. The crude was purified by prep HPLC (Method L) to obtain the diastereomeric mixture of tert-butyl (4aS,8aS)-4-((2-(5-(2-(diisopropylcarbamoyl)-4-fluorophenoxy)pyrimidin-4-yl)-5-oxa-2-azaspiro[3.4]octan-6-yl)methyl)hexahydro-2H-pyrido[4,3-b][1,4]oxazine-6(5H)-carboxylate (13 mg. 15.69% yield) as a solid. LCMS (Method D): Rt=2.09 min, m/z=683.6 [M+H]⁺, 85.88%.

### Step 5. 5-Fluoro-N,N-diisopropyl-2-((4-(6-(((4aS,8aS)-octahydro-4H-pyrido[4,3-b][1,4]oxazin-4-yl)methyl)-S-oxa-2-azaspiro[3.4]octan-2-yl)pyrimidin-5-yl)oxy)benzamide

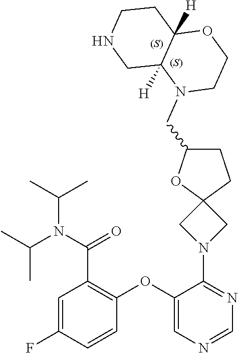

This compound was synthesized following the procedure described for the synthesis of Example 56, Step 5, starting with 1 equivalent of tert-butyl (4aS,8aS)-4-((2-(5-(2-(diisopropylcarbamoyl)-4-fluorophenoxy)pyrimidin-4-yl)-5-oxa-2-azaspiro[3.4]octan-6-yl)methyl)hexahydro-2H-pyrido[4,3-b][1,4]oxazine-6(5H)-carboxylate, except that 30 equivalents of TFA was used. After completion, the reaction was concentrated under reduced pressure. The crude was purified by prep HPLC (Method P) to obtain the diastereomeric mixture of 5-fluoro-N,N-diisopropyl-2-((4-(6-(((4aS,8aS)-octahydro-4H-pyrido[4,3-b][1,4]oxazin-4-yl)methyl)-5-oxa-2-azaspiro[3.4]octan-2-yl)pyrimidin-5-yl)oxy)benzamide as a solid.

Yield: 21.97%; ¹H NMR (400 MHz, DMSO-d₆): δ 8.33-8.28 (m, 1H), 7.81-7.74 (m, 1H), 7.29-7.18 (m, 2H), 7.06-6.96 (m, 1H), 4.28-3.98 (m, 5H), 3.73-3.62 (m, 2H), 3.59-3.47 (m, 3H), 3.24-3.16 (m, 1H), 3.11-3.02 (m, 1H), 2.96-2.74 (m, 2H), 2.67-2.61 (m, 1H), 2.32-2.27 (m, 1H), 2.26-2.19 (m, 1H), 2.18-2.06 (m, 3H), 2.04-1.88 (m, 3H), 1.72-1.62 (m, 1H), 1.60-1.49 (m, 1H), 1.44 (d, J=6.6 Hz, 3H), 1.34 (br d, J=6.6 Hz, 3H), 1.09 (d, J=6.6 Hz, 3H), 1.04-0.95 (m, 3H), one proton merged with solvent peaks; LCMS (Method D): Rt=1.51 min, m/z=583.6 [M+H]⁺; HPLC (Method A): Rt=4.75 min, 97.67%.

### Example 61. 5-Fluoro-N-isopropyl-N-((1r,3r)-3-methyl-3-(pyrrolidin-1-yl)cyclobutyl)-2-((4-(6-(((4aS,8aS)-6-(methyl-da)octahydro-4K-pyrido[4,3-b][1,4]oxazin-4-yl)methyl)-2-azaspiro[3.3]heptan-2-yl)pyrimidin-5-yl)oxy)benzamide

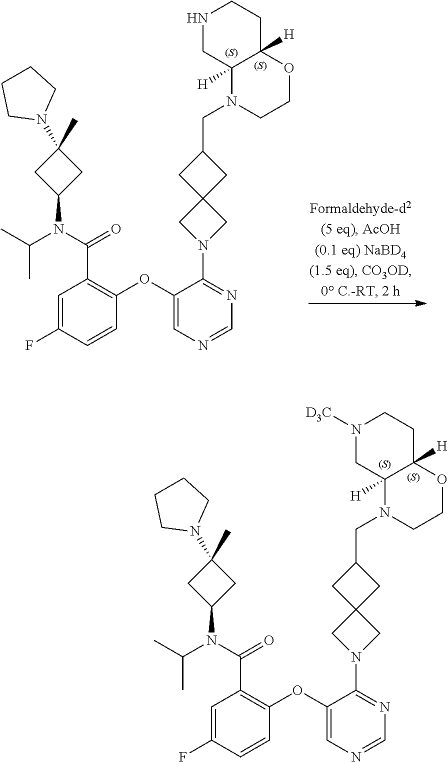

To a stirred solution of 5-fluoro-N-isopropyl-N-((1r,3r)-3-methyl-3-(pyrrolidin-1-yl)cyclobutyl)-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide (0.18 g, 0.27 nmol) in CD₃OD (2 mL) at 0° C. was added formaldehyde-d₂ (20% in D₂O solution, 0.20 mL, 1.36 mmol) and AcOH (1.56 μL, 0.03 mmol). The reaction was stirred at RT for 1 h, then cooled to 0° C. and NaBD₄ (0.02 g, 0.41 mmol) was added. The reaction was stirred at RT for 30 min and monitored by LCMS. After completion, the reaction was quenched with aqueous NaHCO₃ solution (10 mL) and extracted with DCM (3×30 mL). The combined organic layer was dried over sodium sulfate and filtered, and the filtrate was concentrated under reduced pressure. The residue was purified by reverse phase chromatography using a SiliCycle SiliaSep C18 column (80 g) on Biotage isolera one (Mobile phase A: 0.1% NH₄HCO₃ in water, B: ACN) to obtain 5-fluoro-N-isopropyl-N-((1r,3r)-3-methyl-3-(pyrrolidin-1-yl)cyclobutyl)-2-((4-(6-(((4aS,8aS)-6-(methyl-da)octahydro-4H-pyrido[4,3-b][1,4]oxazin-4-yl)methyl)-2-azaspiro[3.3]heptan-2-yl)pyrimidin-5-yl)oxy)benzamide (117 mg, 62.1% yield) as a solid. ¹H NMR (400 MHz, DMSO-d₆): δ 8.25 (s, 1H), 7.83-7.52 (m, 1H), 7.32-6.96 (m, 3H), 4.31-3.93 (m, 5H), 3.91-3.62 (m, 3H), 3.55-3.42 (m, 1H), 3.10-2.78 (m, 3H), 2.76-2.69 (m, 1H), 2.67-2.58 (m, 2H), 2.42-2.37 (m, 1H), 2.31-2.02 (m, 7H), 2.00-1.39 (m, 15H), 1.37-1.23 (m, 3H), 1.13-0.88 (m, 5H); LCMS (Method D): Rt=1.54 min, m/z=679.4 [M+H]⁺, 99.74%. HPLC (Method H): Rt=8.19 min, 98.01%. Chiral SFC (Method AJ): Rt=2.62 min, 98.80%/s.

### Example 62. 2-Methyl-1-(pivaloyloxy)propyl (4aS,8S)-4-((2-(5-(4-fluoro-2-(isopropyl((1r,3r)-3-methyl-3-(pyrrolidin-1-yl)cyclobutyl)carbamoyl)phenoxy)pyrimidin-4-yl)-2-azaspiro[3.3]heptan-6-yl)methyl)hexahydro-2H-pyrido[4,3-b][1,4]oxazine-6(5H)-carboxylate

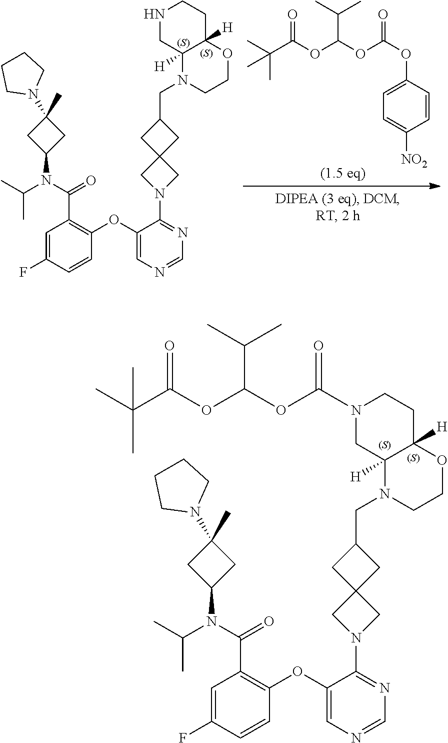

To a stirred solution of 5-fluoro-N-isopropyl-N-((1r,3r)-3-methyl-3-(pyrrolidin-1-yl)cyclobutyl)-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzamide (0.18 g, 0.27 mmol) and 2-methyl-1-(((4-nitrophenoxy)carbonyl)oxy)propyl pivalate (0.14 g, 0.41 mmol) in DCM (5 mL) at RT under nitrogen was added DIPEA (0.14 mL, 0.82 mmol)). The reaction was stirred at RT for 2 h, and monitored by LCMS. After completion, the reaction was quenched with 10/NaOH solution (30 mL) and extracted with DCM (3×30 mL). The combined organic layer was dried over sodium sulfate and filtered, and the filtrate was concentrated under reduced pressure. The residue was purified by reverse phase chromatography using a SiliCycle SiliaSep C18 column (80 g) on Biotage isolera one (Mobile phase A: 0.1% FA in water, B: ACN) to obtain 2-methyl-1-(pivaloyloxy)propyl (4aS,8aS)-4-((2-(5-(4-fluoro-2-(isopropyl((1r,3r)-3-methyl-3-(pyrrolidin-1-yl)cyclobutyl)carbamoyl)phenoxy)pyrimidin-4-yl)-2-azaspiro[3.3]heptan-6-yl)methyl)hexahydro-2H-pyrido[4,3-b][1,4]oxazine-6(5H)-carboxylate (65 mg, 26.6% yield) as a solid. ¹H NMR (400 MHz, DMSO-d₆): δ 8.32-8.18 (m, 1H), 7.82-7.52 (m, 1H), 7.36-6.98 (m, 3H), 6.50-6.27 (m, 1H), 4.34-3.80 (m, 7H), 3.72 (br s, 2H), 3.51 (s, 1H), 3.22-3.11 (m, 1H), 3.00-2.76 (m, 2H), 2.74-2.68 (m, J=5.4 Hz, 1H), 2.65-2.53 (m, 3H), 2.42-2.35 (m, 2H), 2.30-2.09 (m, 6H), 2.06-1.93 (m, 2H), 1.90-1.58 (m, 9H), 1.49-1.21 (m, 6H), 1.13 (s, 9H), 0.91 (d, J=6.8 Hz, 1H); LCMS (Method G): Rt=1.49 min, m/z 862.5 [M+H]⁺, 97.57%. HPLC (Method I): Rt=7.36 min, 96.12%. Chiral SFC (Method AK): Rt=2.55 min, 99.09%.

### Example 63. 5-Fluoro-N-isopropyl-N-((2s,4r)-5-methyl-5-azaspiro[3.4]octan-2-yl)-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamide

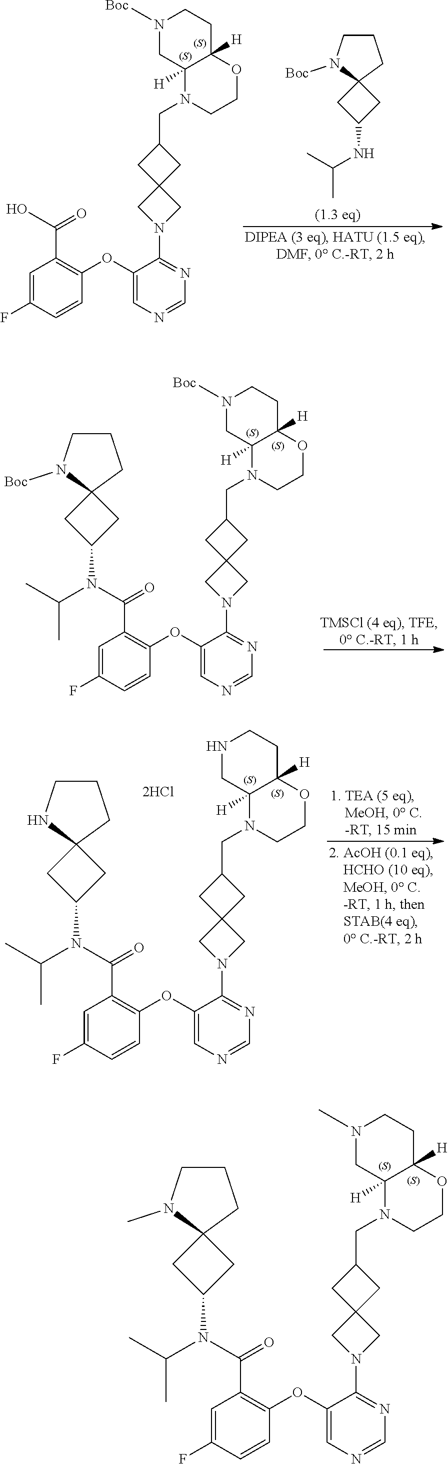

### Step 1. tert-Butyl (4aS,8aS)-4-((2-(5-(2-(((2s,4r)-5-(tert-butoxycarbonyl)-5-azaspiro[3.4]octan-2-yl)(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate

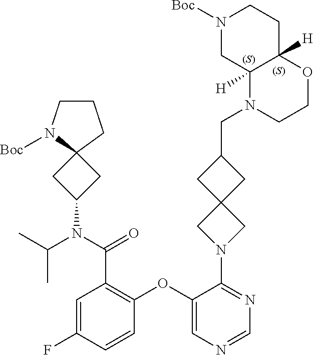

To a stirred solution of 2-((4-(6-(((4aS,8aS)-6-(tert-butoxycarbonyl)octahydro-4H-pyrido[4,3-b][1,4]oxazin-4-yl)methyl)-2-azaspiro[3.3]heptan-2-yl)pyrimidin-5-yl)oxy)-5-fluorobenzoic acid (600 mg, 1.03 mmol), tert-butyl (2s,4r)-2-(isopropylamino)-5-azaspiro[3.4]octane-5-carboxylate (359 mg, 1.34 mmol), and DIPEA (0.54 mL, 3.08 mmol) in DMF (10 mL) at 0° C. was added HATU (586 mg, 1.54 mmol). The reaction was stirred for at RT for 2 h, and monitored by LCMS. After completion, the reaction was diluted with water (20 mL) and extracted with EtOAc (2×20 mL). The combined organic layer was dried over anhydrous sodium sulfate and filtered, and the filtrate was concentrated under reduced pressure. The residue was purified by silica gel flash column chromatography using MeOH in DCM (product eluted at 14% MeOH in DCM) to obtain tert-butyl (4aS,8aS)-4-((2-(5-(2-(((2s,4r)-5-(tert-butoxycarbonyl)-5-azaspiro[3.4]octan-2-yl)(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (720 mg, 82% yield) as a solid. LCMS (Method D): Rt=2.51 min, m/z=834.3 [M+H]⁺, 97.47%.

### Step 2. 5-Fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)-N-((2s,4r)-5-azaspiro[3.4]octan-2-yl)benzamide dihydrochloride

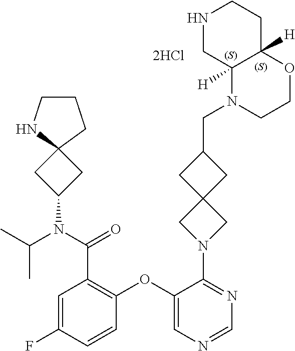

To a stirred solution of tert-butyl (4aS,8aS)-4-((2-(5-(2-(((2s,4r)-5-(tert-butoxycarbonyl)-5-azaspiro[3.4]octan-2-yl)(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (720 mg, 0.86 mmol) in TFE (10 mL) at 0° C. was added TMSCl (0.44 mL, 3.45 mmol). The reaction was stirred at RT for 1 h, and monitored by TLC (10% MeOH in DCM). After completion, the reaction was concentrated under reduced pressure, and the residue was triturated with EtOAc (5 mL). The organic layer was decanted and the residue was dried over reduced pressure to obtain crude 5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)-N-((2s,4r)-5-azaspiro[3.4]octan-2-yl)benzamide dihydrochloride (700 mg, quantitative yield) as a solid. This material was used without further purification. LCMS (Method D): Rt=1.41 min, m/z=634.3 [M+H]⁺, 96.50%.

### Step 3. 5-Fluor-N-isopropyl-N-((2s,4r)-5-methyl-5-azaspiro[3.4]octan-2-yl)-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamide

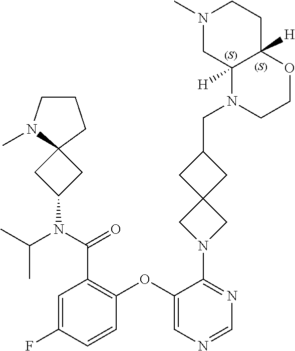

To a stirred solution of 5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)-N-((2s,4r)-5-azaspiro[3.4]octan-2-yl)benzamide dihydrochloride (700 mg, 1.10 mmol) in MeOH (10 mL) was added TEA (0.77 mL, 5.52 mmol). The reaction was stirred at RT for 15 min then concentrated under reduced pressure. The residue was dissolved in MeOH (10.0 mL), and formaldehyde (37% in H₂O; 0.82 mL, 11.04 mmol) and AcOH (6.32 μL, 0.110 mmol) were added at RT. The reaction was stirred at RT for 1 h then cooled to 0° C., and STAB (936 mg, 4.42 mmol) was added. The reaction was stirred at RT for 2 h, and monitored by TLC (10% MeOH in DCM). After completion, the reaction was concentrated under reduced pressure. The residue was diluted with aqueous NaHCO₃ solution (70 mL) and the mixture was extracted with 10% MeOH in DCM (3×70 mL). The combined organic layer was dried over sodium sulfate and filtered, and the filtrate was concentrated under reduced pressure. The residue was purified by reverse phase chromatography using a SiliCycle SiliaSep C18 column (80 g) on Biotage isolera one (Mobile phase A: 0.1% NH₄HCO₃ in water, B: ACN) to obtain 5-fluoro-N-isopropyl-N-((2s,4r)-5-methyl-5-azaspiro[3.4]octan-2-yl)-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamide (425 mg, 57.3% yield) as a solid ¹H NMR (400 MHz, DMSO-d₆): δ 8.32-8.19 (m, 1H), 7.85-7.54 (m, 1H), 7.32-6.99 (m, 3H), 4.31-3.87 (m, 5H), 3.80-3.62 (m, 3H), 3.48 (br t, J=11.4 Hz, 1H), 3.11-2.86 (m, 3H), 2.79-2.70 (m, 1H), 2.65-2.57 (m, 2H), 2.39-2.34 (m, 2H), 2.30-2.14 (m, 8H), 2.10-1.87 (m, 7H), 1.85-1.43 (m, 1H), 1.32 (br d, J=6.4 Hz, 1H), 1.12-0.89 (m, 4H); LCMS (Method D): Rt=1.56 min, m/z=662.3 [M+H]⁺, 98.63%. HPLC (Method H): Rt=7.99 min, 98.50%. Chiral SFC (Method AL): Rt=3.75 min, 100%.

### Example 64. 5-Fluoro-N-isopropyl-N-((2s,4r)-5-methyl-5-azaspiro[3.5]nonan-2-yl)-2-((4-(6-(((4aS,8aS)-6-methyloctahydro-4H-pyrido[4,3-b][1,4]oxazin-4-yl)methyl)-2-azaspiro[3.3]heptan-2-ylpyrimidin-5-yl)oxy)benzamide

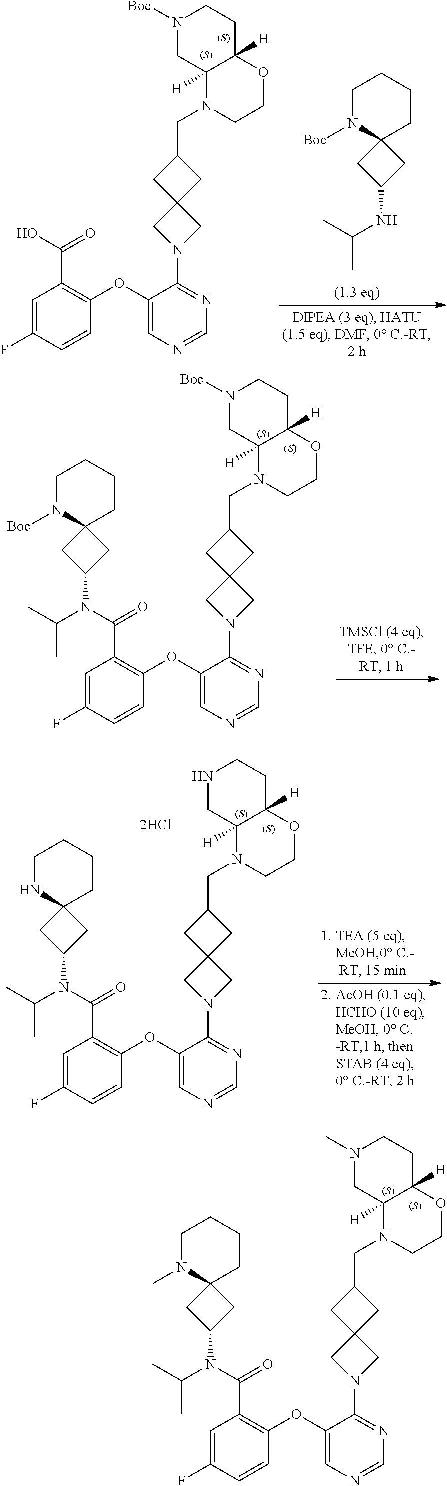

### Step 1. tert-Butyl (4aS,8aS)-4-((2-(5-(2-(((2s,4r)-5-(tert-butoxycarbonyl)-5-azaspiro[3.5]nonan-2-yl)(isopropyl)carbamoyl)-4-fluorophenoxy)pyrimidin-4-yl-2-azaspiro[3.3]heptan-6-yl)methyl)hexahydro-2H-pyrido[4,3-b][1,4]oxazine-6(5H)-carboxylate

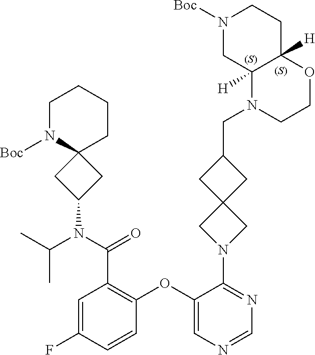

To a stirred solution of 2-((4-(6-(((4aS,8aS)-6-(tert-butoxycarbonyl)octahydro-4H-pyrido[4,3-b][1,4]oxazin-4-yl)methyl)-2-azaspiro[3.3]heptan-2-yl)pyrimidin-5-yl)oxy)-5-fluorobenzoic acid (600 mg, 1.03 mmol), tert-butyl (2s,4r)-2-(isopropylamino)-5-azaspiro[3.5]nonane-5-carboxylate (377 mg, 1.34 mmol), and DIPEA (0.54 mL, 3.08 mmol) in DMF (6 mL) at 0° C. was added HATU (586 mg, 1.54 mmol). The reaction was stirred at RT for 2 h, and monitored by LCMS. After completion, the reaction was diluted with water (30 mL) and extracted with EtOAc (3×20 mL). The combined organic layer was washed with brine (10 mL), dried over anhydrous sodium sulfate and filtered, and the filtrate was concentrated under reduced pressure. The residue was purified by silica gel flash column chromatography using MeOH in DCM (product eluted at 9% MeOH in DCM) to obtain tert-butyl (4aS,8aS)-4-((2-(5-(2-(((2s,4r)-5-(tert-butoxycarbonyl)-5-azaspiro[3.5]nonan-2-yl)(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (820 mg, 84% yield) as a solid. LCMS (Method D): Rt=2.59 min, m/z=848.3 [M+H]⁺, 89.66%.

### Step 2. 5-Fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)(oxy)-N-((2s,4r)-5-azaspiro[3.5]nonan-2-yl)benzamide dihydrochloride

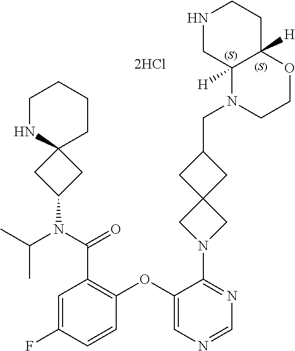

To a stirred solution of tert-butyl (4aS,8aS)-4-((2-(5-(2-(((2s,4r)-5-(tert-butoxycarbonyl)-5-azaspiro[3.5]nonan-2-yl)(isopropyl)carbamoyl)-4-fluorophenoxy)pyrimidin-4-yl)-2-azaspiro[3.3]heptan-6-yl)methyl)hexahydro-2H-pyrido[4,3-b][1,4]oxazine-6(5H)-carboxylate (790 mg, 0.93 mmol) in TFE (10 mL) at 0° C. was added TMSCl (0.48 mL, 3.73 mmol). The reaction was stirred at RT for 1 h, and monitored by LCMS. After completion, the reaction was concentrated under reduced pressure, and the residue was triturated with EtOAc (2×10 mL). The organic layer was decanted and the residue was dried under reduced pressure to obtain 5-fluoro-N-isopropyl-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)-N-((2s,4r)-5-azaspiro[3.5]nonan-2-yl)benzamide dihydrochloride (800 mg, 98% yield) as solid. This material was used without further purification. LCMS (Method D): Rt=1.48 min, m/z=648.3 [M+H]⁺, 74.08%.

### Step 3. S-Fluoro-N-isopropyl-N-((2s,4r)-5-methyl-5-azaspiro[3.5]nonan-2-yl)-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamide

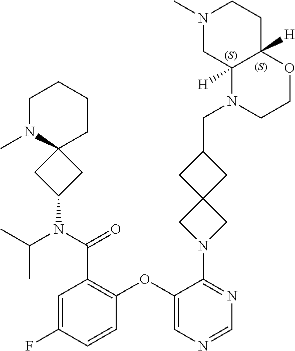

To a stirred solution of 5-fluoro-N-isopropyl-2-((4-(6-(((4aS,8aS)-octahydro-4H-pyrido[4,3-h][1,4]oxazin-4-yl)methyl)-2-azaspiro[3.3]heptan-2-yl)pyrimidin-5-yl)oxy)-N-((2s,4r)-5-azaspiro[3.5]nonan-2-yl)benzamide dihydrochloride (800 mg, 1.23 mmol) in MeOH (10 mL) was added TEA (0.86 mL, 6.17 mmol). The reaction was stirred at RT for 15 min then concentrated under reduced pressure. The residue was dissolved in MeOH (10.00 mL), and formaldehyde (37% in H₂O; 0.92 mL, 12.35 mmol) and AcOH (7.07 μL, 0.12 mmol) were added at RT. The reaction was stirred at RT for 1 h then cooled to 0° C., and STAB (1047 mg, 4.94 mmol) was added. The reaction was stirred at RT for 2 h, and monitored by TLC (10% MeOH in DCM). After completion, the reaction was concentrated under reduced pressure. The residue was diluted with aqueous NaHCO₃ solution (70 mL) and the mixture was extracted with 10% MeOH in DCM (3×70 mL). The combined organic layer was dried over sodium sulfate and filtered, and the filtrate was concentrated under reduced pressure. The residue was purified by reverse phase chromatography using a SiliCycle SiliaSep C18 column (80 g) on Biotage isolera one (Mobile phase A: 0.1% NH₄HCO₃ in water, B: ACN) to obtain 5-fluoro-N-isopropyl-N-((2s,4r)-5-methyl-5-azaspiro[3.5]nonan-2-yl)-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamide (248 mg, 29.2% yield) as a solid. ¹H NMR (400 MHz, DMSO-d₆) δ 8.33-8.20 (m, 1H), 7.85-7.53 (m, 1H), 7.35-6.97 (m, 3H), 4.34-3.87 (m, 4H), 3.79-3.61 (m, 3H), 3.55-3.42 (m, 1H), 3.10-2.99 (m, 1H), 2.97-2.86 (m, 1H), 2.82-2.61 (m, 4H), 2.35-2.23 (m, 7H), 2.17 (s, 3H), 2.10-2.03 (m, 1H), 2.02-1.98 (m, 1H), 1.95-1.88 (m, 2H), 1.84-1.73 (m, 4H), 1.68-1.55 (m, 2H), 1.50-1.30 (m, 8H), 1.12-0.90 (m, 4H); LCMS (Method B): Rt=1.66 min, m/z=676.2 [M+H]⁺, 97.66%. HPLC (Method H): Rt=8.28 min, 98.37%. Chiral SFC (Method AM): Rt=3.55 min, 100%.

### Example 65. 5-Fluoro-N-isopropyl-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)-N-((2s,4r)-5-azaspiro[3.5]nonan-2-yl)benzamide

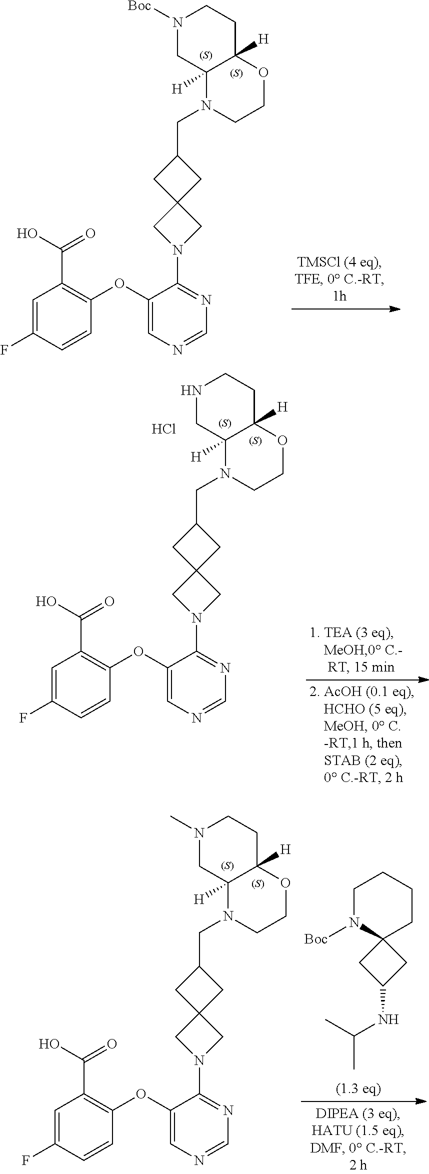

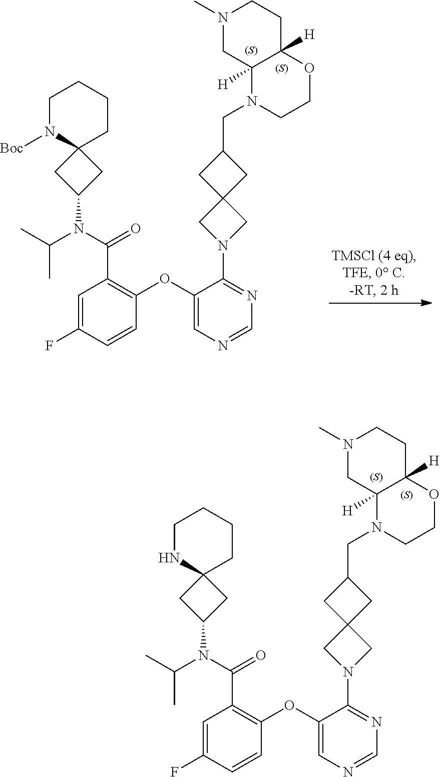

### Step 1. 5-Fluoro-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzoic acid hydrochloride

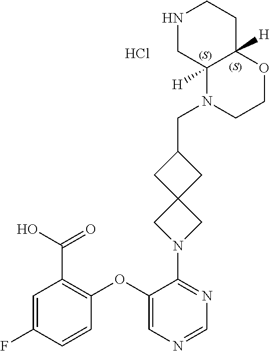

To a stirred solution of 2-((4-(6-(((4aS,8aS)-6-(tert-butoxycarbonyl)octahydro-4H-pyrido[4,3-b][1,4]oxazin-4-yl)methyl)-2-azaspiro[3.3]heptan-2-yl)pyrimidin-5-yl)oxy)-5-fluorobenzoic acid (600 mg, 1.03 mmol) in TFE (10 mL) at 0° C. was added TMSCl (0.53 mL, 4.11 mmol). Then reaction was stirred at RT for 1 h, and monitored by TLC (10% MeOH in DCM). After completion, the reaction was concentrated under reduced pressure, and the residue was triturated with EtOAc (2×10 mL). The organic layer was discarded and remaining residue was dried under vacuum to obtain crude 5-fluoro-2-((4-(6-(((4aS, octahydro-4H-pyrido[4,3-b][1,4]oxazin-4-yl)methyl)-2-azaspiro[3.3]heptan-2-yl)pyrimidin-5-yl)oxy)benzoic acid hydrochloride (600 mg, quantitative yield) as a solid. This material was used without further purification. LCMS (Method D): Rt=1.12 min, m/z=484.2 [M+H]+, 96.08%.

### Step 2. 5-Fluoro-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzoic acid

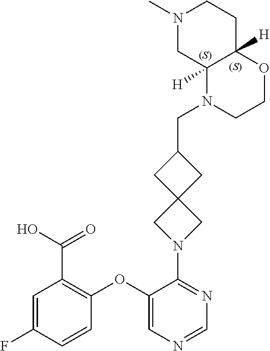

To a stirred solution of 5-fluoro-2-((4-(6-(((4aS,8aS)-octahydro-4H-pyrido[4,3-b][1,4]oxazin-4-yl)methyl)-2-azaspiro[3.3]heptan-2-yl)pyrimidin-5-yl)oxy)benzoic acid hydrochloride (600 mg, 1.15 mmol) in MeOH (5 mL) was added TEA (350 mg, 3.46 mmol). The reaction was stirred at RT for 15 min, then was concentrated under reduced pressure. The residue was dissolved in MeOH (5.00 mL), and formaldehyde (37% in H₂O; 0.43 mL, 5.77 mmol) and AcOH (6.93 mg, 0.11 mmol) were added at RT. The reaction was stirred at RT for 1 h, then cooled to 0° C., and STAB (489 mg, 2.308 mmol) was added. The reaction was stirred at RT for 2 h, and monitored by TLC (10% MeOH in DCM). After completion, the reaction was concentrated under reduced pressure. The residue was purified by reverse phase chromatography using a SiliCycle SiliaSep C18 column (80 g) on Biotage isolera one (Mobile phase A: 0.1% NH₄HCO₃ in water, B: ACN) to obtain 5-fluoro-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzoic acid (380 mg, 65.5% yield) as a solid. LCMS (Method D): Rt=1.14 min, m/z=498.2 [M+H]⁺, 98.96%.

### Step 3. tert-Butyl (2s,4r)-2-(5-fluoro-N-isopropyl-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamido)-5-azaspiro[3.5]nonane-5-carboxylate

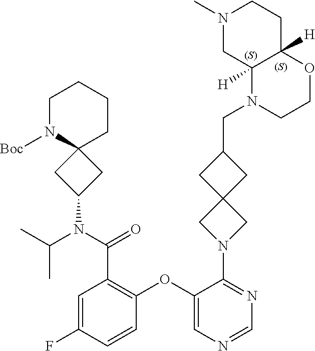

To a stirred solution of 5-fluoro-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzoic acid (370 mg, 0.74 mmol), tert-butyl (2s,4r)-2-(isopropylamino)-5-azaspiro[3.5]nonane-5-carboxylate (273 mg, 0.97 mmol), and DIPEA (0.390 mL, 2.23 mmol) in DMF (4 mL) at 0° C. was added HATU (424 mg, 1.11 mmol). The reaction was stirred at RT for 2 h, and monitored by TLC (10% MeOH in DCM). After completion, the reaction was concentrated under reduced pressure. The residue was dissolved in EtOAc (20 mL), water (20 mL) was added, and the layers were separated. The aqueous layer was extracted with EtOAc (3×20 mL). The combined organic layer was dried over sodium sulfate and filtered, and the filtrate was concentrated under reduced pressure. The residue was purified by reverse phase chromatography using a SiliCycle SiliaSep C18 column (40 g) on Biotage isolera one (Mobile phase A: 0.1% NH₄HCO₃ in water, B: ACN) to obtain tert-butyl (2s,4r)-2-(5-fluoro-N-isopropyl-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamido)-5-azaspiro[3.5]nonane-5-carboxylate (350 mg, 44.7% yield) as a solid. LCMS (Method D): Rt=2.52 min, m/z=777.5 [M+H]⁺, 72.42%.

### Step 4. 5-Fluoro-N-isopropyl-2-((4-(6-(((4aS,8aS,)-6-methyloctahydro-4H-pyrido[4,3-b][1,4]oxazin-4-yl)methyl)-2-azaspiro[3.3]heptan-2-yl)pyrimidin-5-yl)oxy)-N-((2s,4r)-5-azaspiro[3.5]nonan-2-yl)benzamide

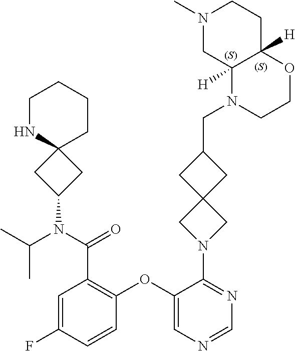

To a stirred solution of tert-butyl (2s,4r)-2-(5-fluoro-N-isopropyl-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)benzamido)-5-azaspiro[3.5]nonane-5-carboxylate (340 mg, 0.45 mmol) in TFE (4 mL) at 0° C. was added TMSCl (0.23 mL, 1.78 mmol). The reaction was stirred at RT for 2 h, and monitored by TLC (10% MeOH in DCM). After completion, the reaction was concentrated under reduced pressure. The residue was dissolved in DCM (4.00 mL) and TEA (0.31 mL, 2.23 mmol) was added. The reaction was stirred for 15 min at RT, then concentrated under reduced pressure. The residue was purified by reverse phase chromatography using a SiliCycle SiliaSep C18 column (80 g) on Biotage isolera one (Mobile phase A: 0.1% NH₄HCO₃ in water, B: ACN) to obtain 5-fluoro-N-isopropyl-2-((4-(6-(((4aS,8aS)-6-methyloctahydro-4H-pyrido[4,3-b][1,4]oxazin-4-yl)methyl)-2-azaspiro[3.3]heptan-2-yl)pyrimidin-5-yl)oxy)-N-((2s,4r)-5-azaspiro[3.5]nonan-2-yl)benzamide (183 mg, 61.9% yield) as a solid. ¹H NMR δ 8.32-8.19 (m, 1H), 7.85-7.67 (m, 1H), 7.38-6.97 (m, 3H), 4.32-3.91 (m, 5H), 3.75-3.61 (m, 2H), 3.58-3.41 (m, 1H), 3.09-3.00 (m, 2H), 2.95-2.83 (m, 1H), 2.75-2.58 (m, 5H), 2.30-2.03 (m, 8H), 1.96-1.77 (m, 8H), 1.68-1.54 (m, 3H), 1.49-1.23 (m, 9H), 1.11-0.89 (m, 3H); LCMS (Method D): Rt=1.55 min, m/z=662.2 [M+H]⁺, 98.94%. HPLC (Method H): Rt=7.70 min, 99.86%. Chiral SFC (Method AN): Rt=3.06 min, 100%.

### Example B-1. Menin-MLL Competition and MV4;11 Cell Proliferation Assays

Menin-MLL is a competition assay between human Menin and N-terminal portion of human MLL representing amino acids 4-43 of the protein. The interaction between Menin and MLL peptide was monitored by HTRF employing Terbium labeled anti-His6 antibody directed to the N-terminal His6-tag on recombinant Menin and FITC group covalently attached to the MLL peptide. The N-terminal fragment of MLL, retained in all MLL fusion proteins, is involved in the interactions with Menin, and this protein-protein interaction is critical for the MLL fusion proteins mediated leukemogenic transformations.

For IC₅₀ determination test compounds were prepared as 10 mM DMSO stock solutions. Considering DMSO as the vehicle in the assay system. Lower sub-stocks of 50 μM were prepared from the 10 mM stock solution. To test the compounds in assay, 3.16-fold serial dilutions are made in 100% DMSO. Mid-stock of 50× compounds (50 μM) were serially diluted (3.16 fold) in 100% DMSO in Polypropylene plate. In assay plate 1 micro-litre of the previously prepared compound dilution was stamped. H-FL-Menin diluted to 4 nM in assay buffer (50 mM Tris-HCl, pH 7.4, 50 mM NaCl, freshly prepared 1 mM DTT, 0.01% BSA, 0.005% Triton X-100) was pre-incubated with 8 nM anti-His6-Tb for 30 min at room temperature. FITC-MLL-4-43 was diluted to 2 nM in assay buffer and 25 μL was dispensed into each well of the assay plate followed by addition of 25 μl of pre-incubated H-FL-Menin and anti-His6-Tb mixture. Final concentration H-FL-Menin diluted to 1 nM in assay plate with 2 nM anti-His6-Tb and 1 nM FITC-MLL-4-43. After 1 hr incubation at room temperature, the HTRF signal was measured on the Spark multi-label plate reader. Resulting data were captured as a ratio of RFU520/RFU485×1000. The max values were obtained from 0% inhibition in presence of 2% DMSO and the min. values were 100% inhibition in presence of 1 μM reference compound.

### Example B-2. Cell Proliferation Assay on MV4;11 and MV4-11 [M327I-Heterozygous] Cells Using CTG Assay

### Test Compounds Preparation

Compounds were dissolved to obtain as 10 mM solution in DMSO. These stocks were diluted 1:5 to the top concentration to achieve 2 mM in 100% DMSO. For EC₅₀ determination, compounds were serially diluted with semi-log in 100% DMSO. Each prepared DMSO solution were further diluted 1:500 in the cell culture media to obtain the 2× dosing solutions. The Final concentrations of tested compounds in the cell culture media ranged from 0.632 nM to 2000 nM which keeps % DMSO in assay as 0.1 across the wells.

### Experimental Procedure

MV4;11 [harboring the MLL-AF4; (ATCC Cat. No. CRL-9591)] & mutated cell line of MV4-11 [M3271-hetero; Source: DFCI, Boston, MA] cells were cultured in IMDM/RPMI with 10% HI FBS at 5% CO₂ and 37° C. The cell suspension containing 150,000 cells/mL in the culture medium was dispensed in 96-well cell culture plates 100 μL (15,000 cell/well) per well. Then 100 μL of 2× dosing media with test compounds were added to each well. Cells were cultured for 72 h at 37° C. and 5% CO₂ in a humidified incubator.

Post 72 h incubation, cultured cells were resuspended and 100 μL of the suspension from the assay plate was transferred into 96-well black OptiPlate (PerkinElmer Cat. No. 6005320). To this, 100 μL of Cell Titer Glo reagent was added, assay plates were shaken for 5 min at RT, and luminescence was read on Tecan Spark (Tecan) plate reader.

### Data Analysis

Percent inhibition values were calculated based on the high luminescence signal obtained with 0.1% DMSO control and the low signal observed with 5 μM Revumenib. The percent inhibition vs. concentration data were fit into a four-parameter model, and EC₅₀ values were calculated from the fit as the concentrations corresponding to the inflection points on the dose-response curves. Data is shown in Table 2.

### Example B-3. Determination of Binding Constants to Menin-MLL

For Ki determination, individual compounds were prepared as 10 mM DMSO stock solutions. Considering DMSO as the vehicle in the assay system. Lower sub-stocks of 16 μM were prepared from the 10 mM stock solution. To test the compounds in assay, 3.16-fold serial dilutions are made in 100% DMSO. Mid-stock of 50× compounds (16 μM) were serially diluted (3.16 fold) in 100% DMSO in Polypropylene plate. In assay plate 1 micro-litre of the previously prepared compound dilution was stamped. H-FL-Menin diluted to 1 nM in assay buffer (50 mM Tris-HCl, pH 7.4, 50 mM NaCl, freshly prepared 1 mM DTT, 0.01% BSA, 0.005% Triton X-100) and pre-incubated with 2 nM anti-His6-Tb for 30 min at room temperature and then 25 μl was dispensed into each well. FITC-ML L-4-43 was diluted to 6.4 nM in assay buffer and 25 μL was dispensed into each well of the assay plate followed by addition of 25 μl of pre-incubated H-FL-Menin and anti-His6-Tb mixture. Final concentration H-FL-Menin diluted to 0.25 nM in assay plate with 0.5 nM anti-His6-Tb and 3.2 nM FITC-MLL-4-43. After 24 hr incubation at room temperature, the HTRF signal was measured on the Spark multi-label plate reader. Resulting data were captured as a ratio of RFU520/RFU485×1000. The max values were obtained from 0% inhibition in presence of 2% DMSO and the min. values were 100% inhibition in presence of 320 nM reference compound. Data is shown in Table 2.

TABLE 2
Shows (i) Menin-MLL binding data according to Homogeneous Time Resolved
Fluorescence (HTRF) assay with (i) individual compound Ki (nM) and (ii) the EC₅₀ (nM) against
MV4;11 and MV4;11 M3271 leukemia cell lines. Ki data (nM) are provided below (“n/a” refers
to data not available; “+” means <1.0 nM; “+” means >1.0 nM and <1.5 nM; and “+” means ≥
1.5 nM). EC₅₀ (nM) against MV4;11 and MV4;11 M3271 data are provided below (“n/a” refers
to data not available; “+++” means <100 nM; “++” means >100 nM and <1000 nM; and “+”
means ≥ 1000 nM).

| | | WT | | MV4;11 |
|---|---|---|---|---|
| Compound | | Menin | MV4;11 | M3271 (het) |
| No. | Structure | Ki (nM) | EC₅₀ (nM) | EC₅₀ (nM) |
| 1 | 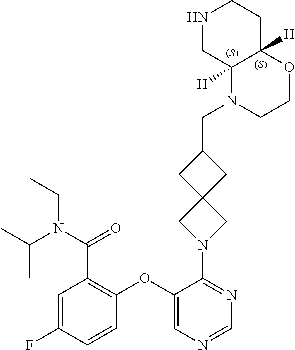 | +++ | +++ | +++ |
| 2 | 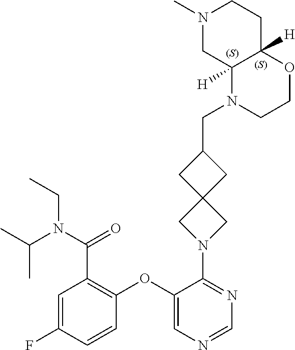 | +++ | +++ | +++ |
| 3 | 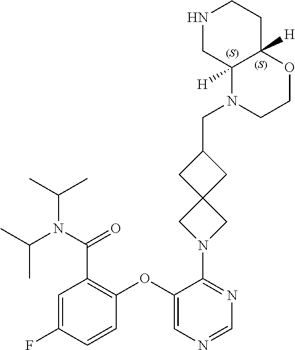 | +++ | +++ | n/a |
| 4 | 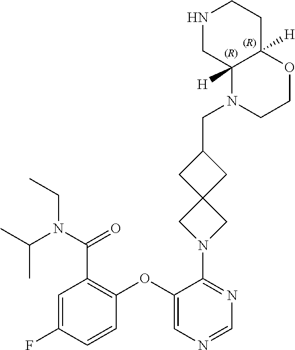 | ++ | ++ | n/a |
| 5 | 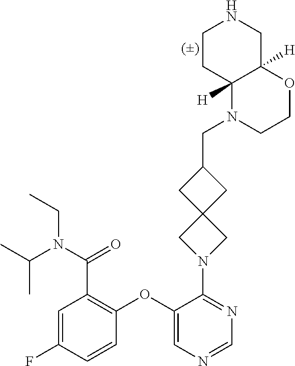 [SMILES: CCN(C(=O)c1cc(F)ccc1Oc1cncnc1N1CC2(CC(CN3CCO[C@@H]4CNCC[C@H]43)C2)C1)C(C)C] | +++ | +++ | n/a |
| 6 | 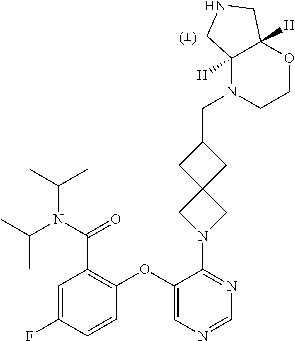 [SMILES: CC(C)N(C(=O)c1cc(F)ccc1Oc1cncnc1N1CC2(CC(CN3CCO[C@H]4CNC[C@@H]43)C2)C1)C(C)C] | +++ | +++ | n/a |
| 7 | 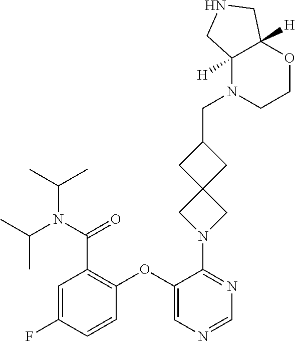 [SMILES: CC(C)N(C(=O)c1cc(F)ccc1Oc1cncnc1N1CC2(CC(CN3CCO[C@H]4CNC[C@@H]43)C2)C1)C(C)C] (Isomer 1) | +++ | +++ | n/a |
| 8 | 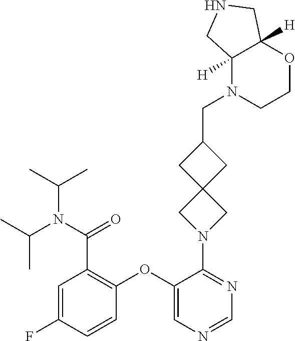 [SMILES: CC(C)N(C(=O)c1cc(F)ccc1Oc1cncnc1N1CC2(CC(CN3CCO[C@H]4CNC[C@@H]43)C2)C1)C(C)C] (Isomer 2) | +++ | +++ | n/a |
| 9 | 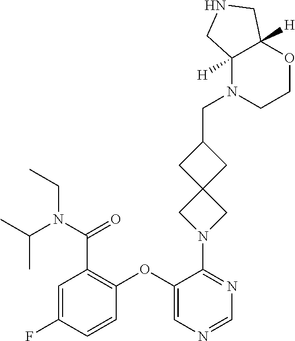 [SMILES: CCN(C(=O)c1cc(F)ccc1Oc1cncnc1N1CC2(CC(CN3CCO[C@H]4CNC[C@@H]43)C2)C1)C(C)C] (Isomer 1) | +++ | ++ | n/a |
| 10 | 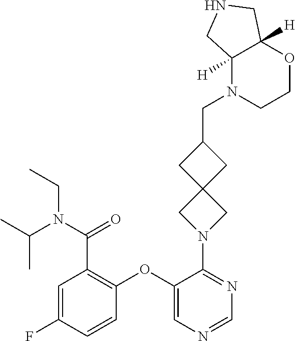 [SMILES: CCN(C(=O)c1cc(F)ccc1Oc1cncnc1N1CC2(CC(CN3CCO[C@H]4CNC[C@@H]43)C2)C1)C(C)C] (Isomer 2) | +++ | +++ | n/a |
| 11 | 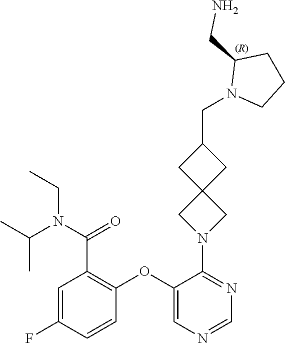 | +++ | ++ | n/a |
| 12 | 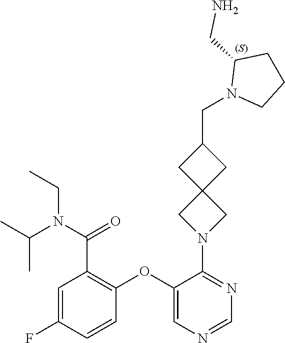 | +++ | ++ | n/a |
| 13 | 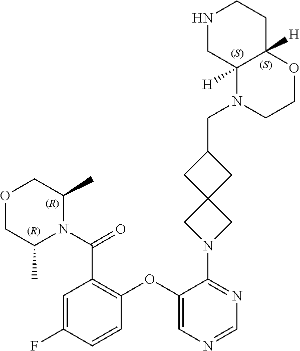 | +++ | +++ | n/a |
| 14 | 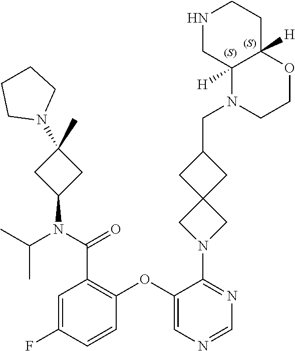 | +++ | +++ | +++ |
| 15 | 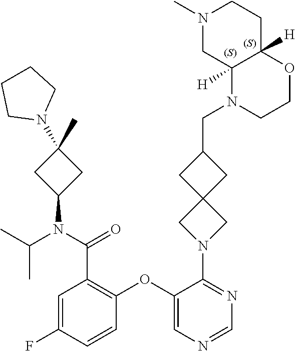 | +++ | +++ | +++ |
| 16 | 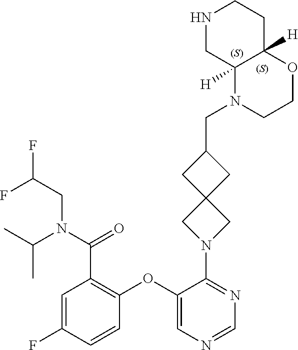 | +++ | +++ | n/a |
| 17 | 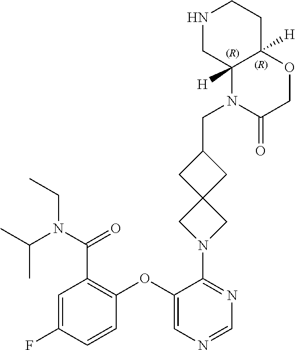 | + | + | n/a |
| 18 | 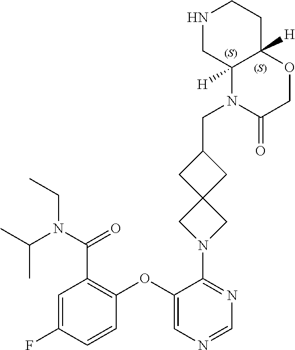 | + | + | n/a |
| 19 | 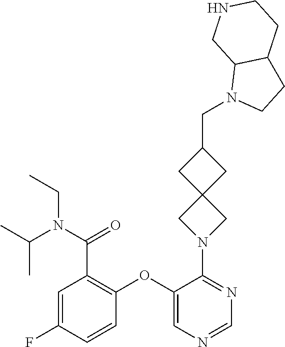 [SMILES: CCN(C(=O)c1cc(F)ccc1Oc1cncnc1N1CC2(CC(CN3CCC4CCNCC43)C2)C1)C(C)C] | +++ | ++ | n/a |
| 20 | 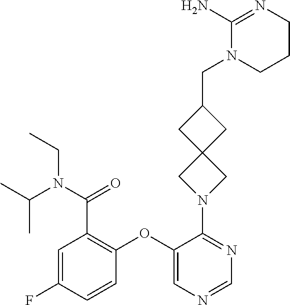 [SMILES: CCN(C(=O)c1cc(F)ccc1Oc1cncnc1N1CC2(CC(CN3CCCN=C3N)C2)C1)C(C)C] | +++ | ++ | n/a |
| 21 | 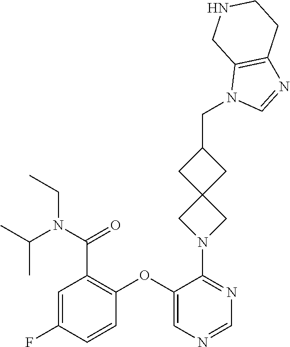 [SMILES: CCN(C(=O)c1cc(F)ccc1Oc1cncnc1N1CC2(CC(Cn3cnc4c3CNCC4)C2)C1)C(C)C] | + | ++ | n/a |
| 22 | 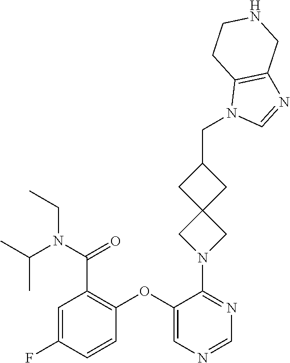 [SMILES: CCN(C(=O)c1cc(F)ccc1Oc1cncnc1N1CC2(CC(Cn3cnc4c3CCNC4)C2)C1)C(C)C] | + | + | n/a |
| 23 | 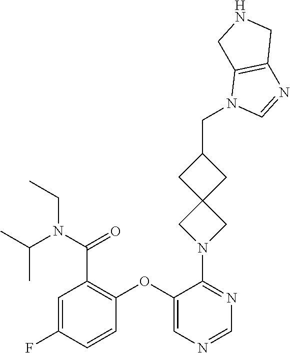 [SMILES: CCN(C(=O)c1cc(F)ccc1Oc1cncnc1N1CC2(CC(Cn3cnc4c3CNC4)C2)C1)C(C)C] | +++ | ++ | n/a |
| 24 | 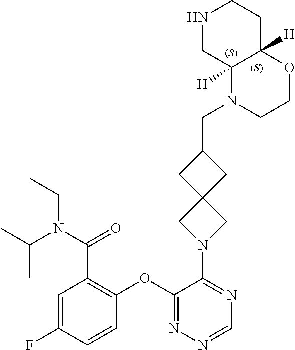 | +++ | +++ | n/a |
| 25 | 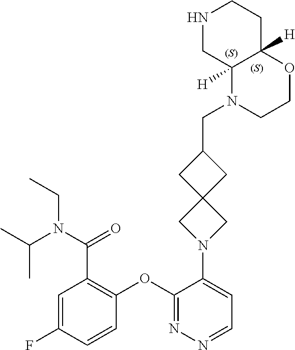 | +++ | +++ | n/a |
| 26 | 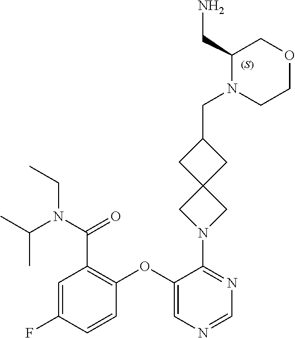 | +++ | +++ | n/a |
| 27 | 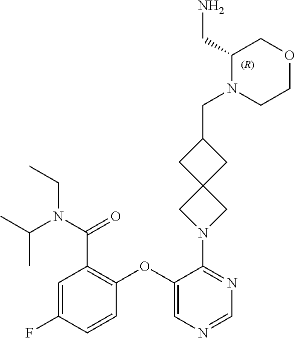 | +++ | ++ | n/a |
| 28 | 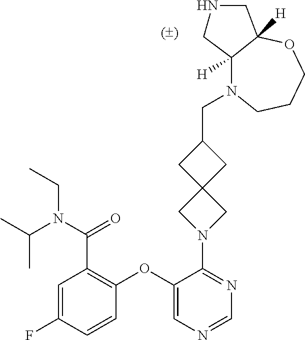 [SMILES: CCN(C(=O)c1cc(F)ccc1Oc1cncnc1N1CC2(CC(CN3CCCO[C@H]4CNC[C@@H]43)C2)C1)C(C)C] | +++ | ++ | n/a |
| 29 | 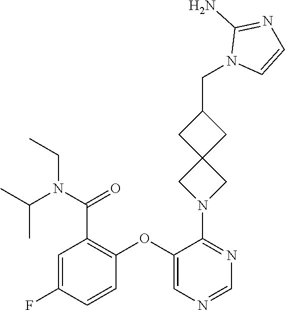 [SMILES: CCN(C(=O)c1cc(F)ccc1Oc1cncnc1N1CC2(CC(Cn3ccnc3N)C2)C1)C(C)C] | +++ | ++ | n/a |
| 30 | 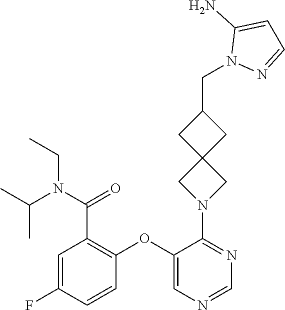 [SMILES: CCN(C(=O)c1cc(F)ccc1Oc1cncnc1N1CC2(CC(Cn3nccc3N)C2)C1)C(C)C] | + | + | n/a |
| 31 | 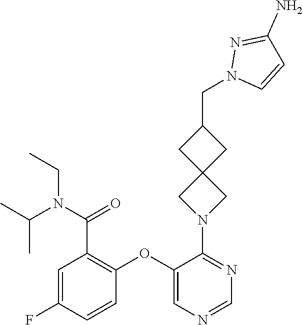 [SMILES: CCN(C(=O)c1cc(F)ccc1Oc1cncnc1N1CC2(CC(Cn3ccc(N)n3)C2)C1)C(C)C] | + | + | n/a |
| 32 | 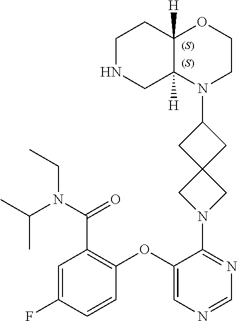 | +++ | +++ | n/a |
| 33 | 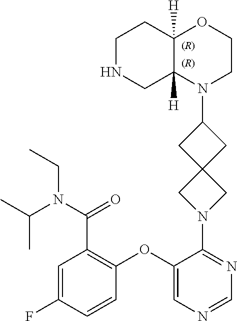 | + | + | n/a |
| 34 | 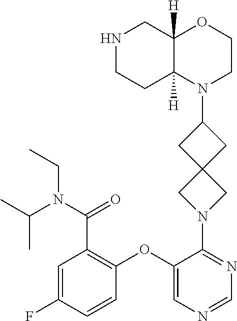 [SMILES: CCN(C(=O)c1cc(F)ccc1Oc1cncnc1N1CC2(CC(N3CCO[C@H]4CNCC[C@@H]43)C2)C1)C(C)C] | + | ++ | n/a |
| 35 | 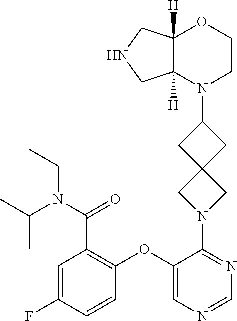 [SMILES: CCN(C(=O)c1cc(F)ccc1Oc1cncnc1N1CC2(CC(N3CCO[C@H]4CNC[C@@H]43)C2)C1)C(C)C] | + | ++ | n/a |
| 36 | 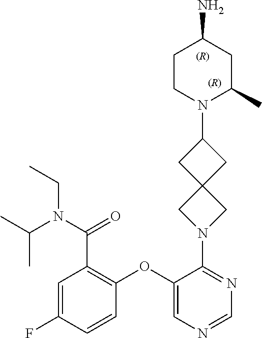 | +++ | +++ | n/a |
| 37 | 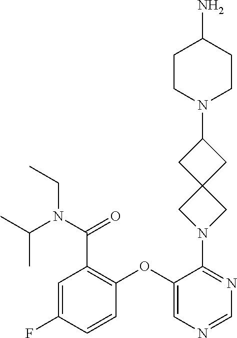 [SMILES: CCN(C(=O)c1cc(F)ccc1Oc1cncnc1N1CC2(CC(N3CCC(N)CC3)C2)C1)C(C)C] | +++ | ++ | n/a |
| 38 | 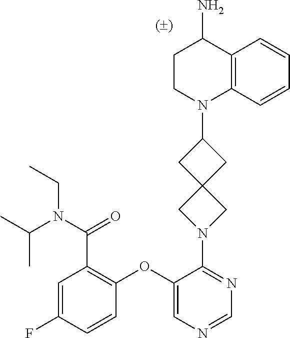 [SMILES: CCN(C(=O)c1cc(F)ccc1Oc1cncnc1N1CC2(CC(N3CCC(N)c4ccccc43)C2)C1)C(C)C] | +++ | +++ | n/a |
| 39 | 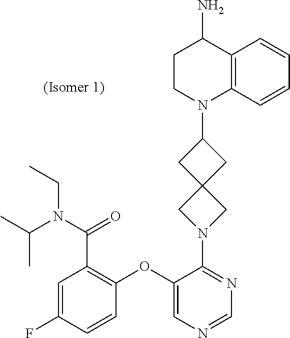 [SMILES: CCN(C(=O)c1cc(F)ccc1Oc1cncnc1N1CC2(CC(N3CCC(N)c4ccccc43)C2)C1)C(C)C] (Isomer 1) | +++ | +++ | n/a |
| 40 | 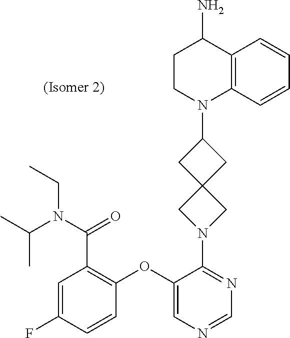 [SMILES: CCN(C(=O)c1cc(F)ccc1Oc1cncnc1N1CC2(CC(N3CCC(N)c4ccccc43)C2)C1)C(C)C] (Isomer 2) | +++ | +++ | n/a |
| 41 | 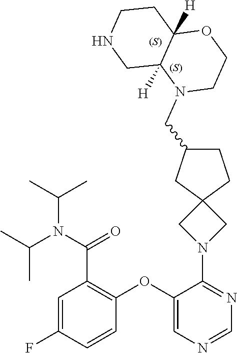 | +++ | +++ | n/a |
| 42 | 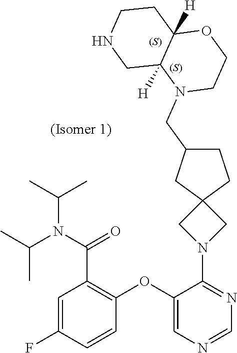 (Isomer 1) | +++ | +++ | n/a |
| 43 | 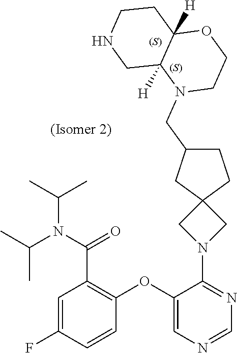 (Isomer 2) | +++ | +++ | +++ |
| 44 | 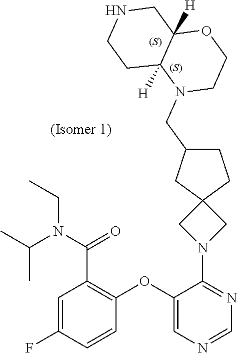 (Isomer 1) | + | ++ | + |
| 45 | 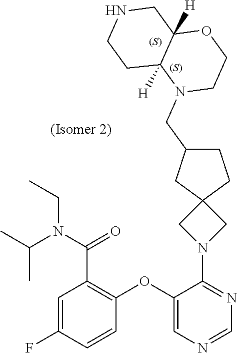 (Isomer 2) | + | ++ | + |
| 46 | 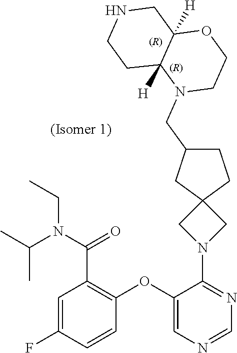 (Isomer 1) | + | ++ | + |
| 47 | 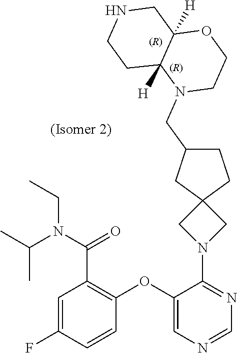 (Isomer 2) | + | ++ | ++ |
| 48 | 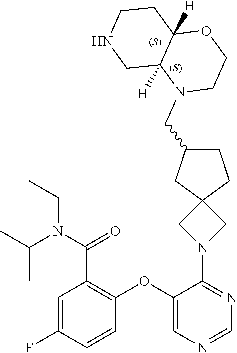 | +++ | +++ | n/a |
| 49 | 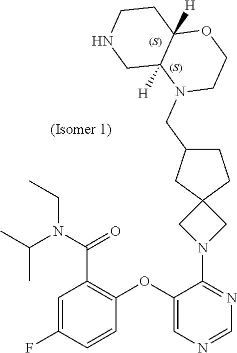 (Isomer 1) | +++ | +++ | ++ |
| 50 | 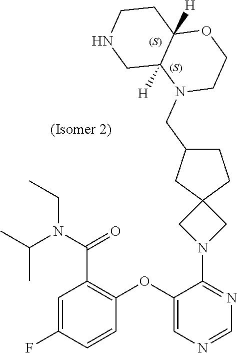 (Isomer 2) | +++ | +++ | ++ |
| 51 | 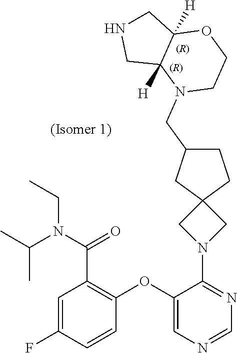 (Isomer 1) | +++ | ++ | n/a |
| 52 | 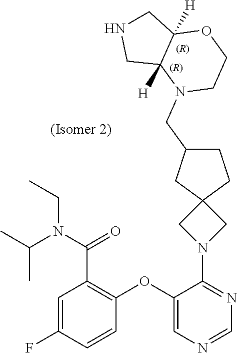 (Isomer 2) | ++ | ++ | n/a |
| 53 | 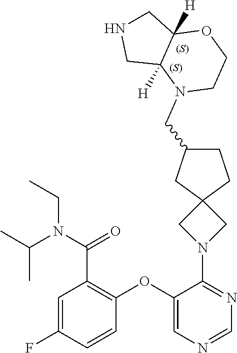 | +++ | +++ | n/a |
| 54 | 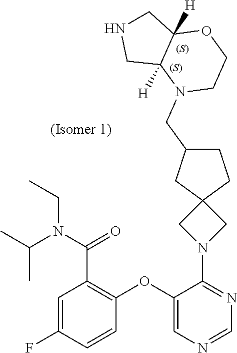 (Isomer 1) | +++ | +++ | n/a |
| 55 | 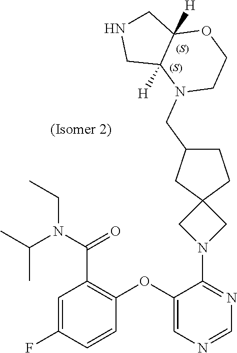 (Isomer 2) | +++ | +++ | n/a |
| 56 | 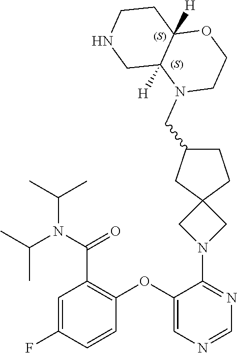 | +++ | +++ | n/a |
| 57 | 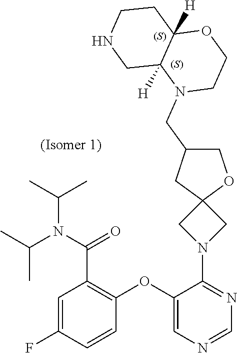 (Isomer 1) | +++ | +++ | +++ |
| 58 | 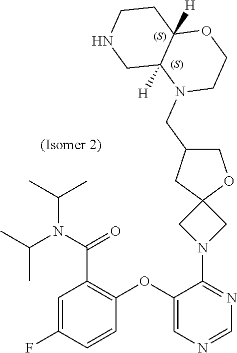 (Isomer 2) | +++ | +++ | n/a |
| 59 | 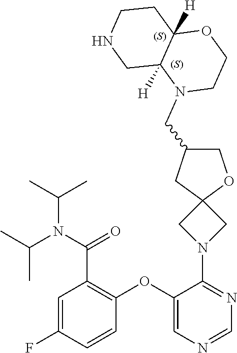 | +++ | +++ | n/a |
| 60 | 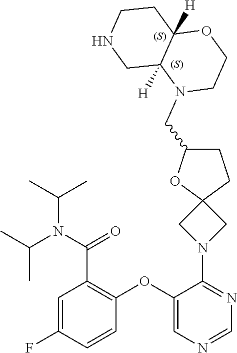 | + | ++ | n/a |
| 61 | 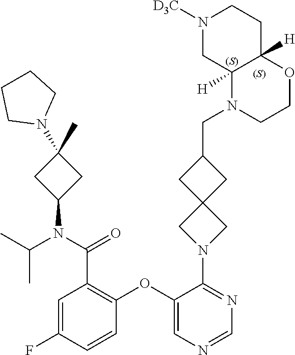 | +++ | +++ | +++ |
| 62 | 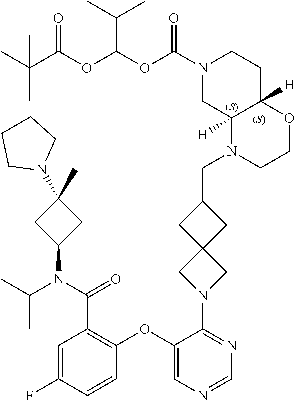 | +++ | +++ | +++ |
| 63 | 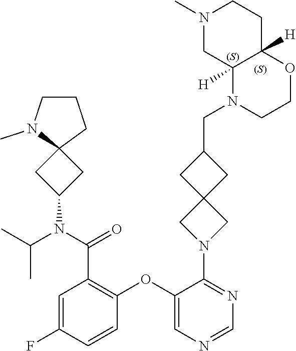 | +++ | +++ | +++ |
| 64 | 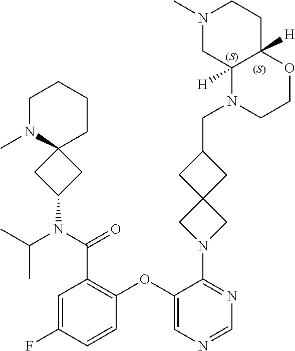 | +++ | +++ | +++ |
| 65 | 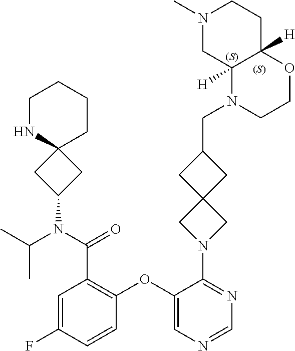 | +++ | +++ | +++ |

### Example B-4. Patch Clamp Assay

The 35 mm culture dishes upon which cells were seeded at a density allowing single cells to be recorded were placed on the dish holder of the microscope and continuously perfused (at approximately 1 mL/min) with the bath solution. All solutions applied to cells including the pipette solution were maintained at room temperature (19° C. to 30° C.). After formation of a Gigaohm seal between the patch electrodes and individual hERG stably transfected HEK 293 cells (pipette resistance range: 2.0 MΩ to 7.0 MΩ; seal resistance range: >1GΩ) the cell membrane across the pipette tip was ruptured to assure electrical access to the cell interior (whole-cell patch-configuration). In case the quality of the seal was poor, the process of seal formation was repeated with a different cell and a new pipette. As soon as a stable seal could be established, hERG outward tail currents were measured upon depolarization of the cell membrane to +20 mV for 2s (activation of channels) from a holding potential of −80 mV and upon subsequent repolarization to −40 mV for 3 s. This voltage protocol (as shown below) was run at least 10 times at intervals of 10 s. If current density was judged to be too low for measurement, another cell was recorded.

Once control recordings have been accomplished, cells were continuously perfused with a bath solution containing the test item 0.3% DMSO or 100 nM E-4031. During wash-in of the test item the voltage protocol indicated above was run continuously again at 10 s intervals until the steady-state level of block was reached.

### EQUIVALENTS

While we have described a number of embodiments of this disclosure, it is apparent that our basic examples may be altered to provide other embodiments that utilize the compounds and methods of this disclosure. The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art. The foregoing description has been presented only for the purposes of illustration and is not intended to limit the disclosure to the precise form disclosed, but by the claims appended hereto. The references cited herein are not admitted to be prior art to the application.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A compound of Formula I, 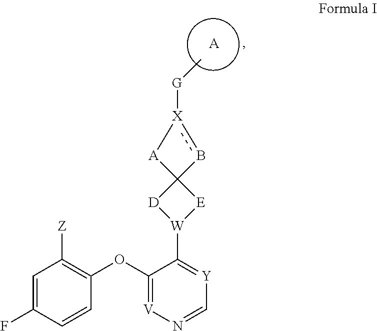
a stereoisomer, or a pharmaceutically acceptable salt thereof, wherein: [symbol]
denotes a single bond or a double bond, as valency permits;
A, B, D, and E are each independently selected from —O—, —C(R^{A1})(R^{A2}), —C(R^{A1})(R^{A2})—C(R^{A1})(R^{A2})—, —C(R^{A1})(R^{A2})—O—, —C(R^{A1})(R^{A2})—NR^{A3}—, —C(═O), —C(R^{A1})(R^{A2})—C(═O)—, and —N═C(NH₂)—, wherein no more than one of A, B, D, and E is —C(R^{A1})(R^{A2})—O—, —C(R^{A1})(R^{A2})—NR^{A3}—, —C(R^{A1})(R^{A2})—C(═O)—, —C(═O)—, or —N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R¹;
G is absent, O, CH₂, or NH, wherein
when G is absent, X is connected with a nitrogen of Ring A;
when G is O, CH₂, or NH, G is connected with the nitrogen of Ring A;
W is N or CR^{W}, wherein R^{W} is H, halo, CN, OH, C₁₋₆ alkyl, C₁₋₆ alkoxy, amino, C₁₋₆ alkyl amino, or C₂₋₈ dialkylamino;
X is N or CR^{X}, wherein R^{X} is absent, H, halo, CN, OH, C₄₋₆alkyl, C₁₋₆alkoxy, amino, C₁₋₆ alkyl amino, or C₂₋₈ dialkylamino;
Z is Cy², halo, C₁₋₆ alkyl, C₁₋₆haloalkyl, C₁₋₆cyanoalkyl, C₂₋₆ alkenyl, C₂₋₆ alkynyl, CN, NO₂, OR^{a3}, SR^{a3}, C(O)R^{b3}, C(O)NR¹R², C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)N^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, and P(O)R^{c3}R^{d3} wherein said C₁₋₆ alkyl, C₁₋₆haloalkyl, C₁₋₆cyanoalkyl, C₂₋₄ alkenyl, and C₂₋₄ alkynyl are each optionally substituted with 1, 2, 3, or 4 substituents independently selected from Cy², halo, CN, NO₂, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3} NR^{c3}C(═NR^{e3})NR^{c3}R^{d3} NR^{c3}R^{d3} NR^{c3}C(O)R^{b3} NR^{c3}C(O)OR^{a3}, NR^{c3}C(O)NR^{c3}R^{d3} NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3};
each R^{A1} is independently selected from absent, H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A2} is independently selected from H, halo, C₁₋₆ alkyl, C₁₋₆ alkoxy, C₁₋₆ haloalkyl, C₁₋₆ haloalkoxy, amino, C₁₋₆ alkylamino, C₂₋₈ dialkylamino, CN, NO₂, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆ alkyl is optionally substituted with phenyl, C₁₋₆ alkoxy, C₁₋₆ haloalkoxy, CN, NO₂, or OH;
R^{z} is H, C₁₋₆ alkyl, or phenyl;
each Cy² is independently selected from C₆₋₁₄ aryl, C₃₋₁₈ cycloalkyl, and 4-18 membered heterocycloalkyl, each of which is optionally substituted with 1, 2, 3, or 4 substituents independently selected from R^{Cy2};
each R^{Cy2} is independently selected from halo, C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, 4-7 membered heterocycloalkyl, CN, NO₂, OR^{a5}, SR^{a5}, C(O)R^{b5}, C(O)NR^{c5}R^{d5}, C(O)OR^{a5}, OC(O)R^{b5}, OC(O)NR^{c5}R^{d5}, C(═NR^{e5})NR^{c5}R^{d5} NR⁵C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5} S(O)R^{b5}, S(O)NR^{c5}R^{d5}, S(O)₂R^{b5}, and S(O)₂NR^{c5}R^{d5}, wherein said C₁₋₆ alkyl, C₁₋₆ haloalkyl, C₁₋₆ cyanoalkyl, C₂₋₆ alkenyl, C₂₋₆ alkynyl, phenyl, C₃₋₇ cycloalkyl, 5-6 membered heteroaryl, and 4-7 membered heterocycloalkyl are each optionally substituted with 1, 2, 3, or 4 substituents independently selected from CN, NO₂, OR^{a5}, SR^{a5}, C(O)R^{b5}, C(O)NR^{c5}R^{d5}, C(O)OR^{a5}OC(O)R^{b5}, OC(O)NR^{c5}R^{d5}, C(═NR^{e5})NR^{c5}R^{d5}, NR^{c5}C(═NR^{e5})NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)R^{b5}, NR^{c5}S(O)₂R^{b5}, NR^{c5}S(O)₂NR^{c5}R^{d5}, S(O)R^{b5}, S(O)NR^{c5}R^{d5}, S(O)₂R^{b5}, and S(O)₂NR^{c5}R^{d5};
R¹ is
(a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
(c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{a3}, R^{b3}, R^{c3}, R^{d3}, R^{a5}, R^{b5}, R^{c5}, and R^{d5} is independently selected from H, C₁₋₆ alkyl, C₁₋₆haloalkyl, C₂₋₆ alkenyl, C₂₋₆ alkynyl, C₆₋₁₀aryl, C₃₋₁₀cycloalkyl, 5-10 membered heteroaryl, 4-10 membered heterocycloalkyl, —C₆₋₁₀aryl-C₁₋₆ alkyl, —C₃₋₁₀cycloalkyl-C₁₋₆alkyl, -(5-10 membered heteroaryl)-C₁₋₆ alkyl, and -(4-10 membered heterocycloalkyl)-C₁₋₆ alkyl, wherein said C₁₋₆ alkyl, C₁₋₆haloalkyl, C₂₋₆ alkenyl, C₂₋₆ alkynyl, C₆₋₁₀aryl, C₃₋₁₀cycloalkyl, 5-10 membered heteroaryl, 4-10 membered heterocycloalkyl, —C₆₋₁₀aryl-C₃₋₁₀ alkyl, —C₃₋₁₀cycloalkyl-C₁₋₆alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
each R^{e3} and R^{c5} is independently selected from H, C₁₋₆ alkyl, and CN;
each R^{g} is independently selected from OH, NO₂, CN, halo, oxo, OBn, N(R_{N})², C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆cycloalkyl, C₁₋₆alkoxy, C₁₋₆ haloalkoxy, cyano-C₁₋₃alkyl, HO—C₁₋₃ alkyl, amino, C₁₋₆ alkylamino, di(C₁₋₆alkyl)amino, thiol, C₁₋₆alkylthio, C₁₋₆alkylsulfinyl, C₁₋₆alkysulfonyl, carboxy, aminocarbonyl, C₁₋₆alkylcarbonyl, and C₁₋₆alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4a}, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

2. The compound of claim 1, a stereoisomer, or a pharmaceutically acceptable salt thereof, wherein when [symbol] is a double bond, X is C.

3. The compound of any one of the preceding claims, a stereoisomer, or a pharmaceutically acceptable salt thereof, wherein the spiro moiety represented by the below formula: 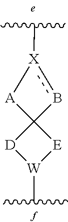
wherein e and f indicate points of attachment to the remainder of the molecule, is selected from:

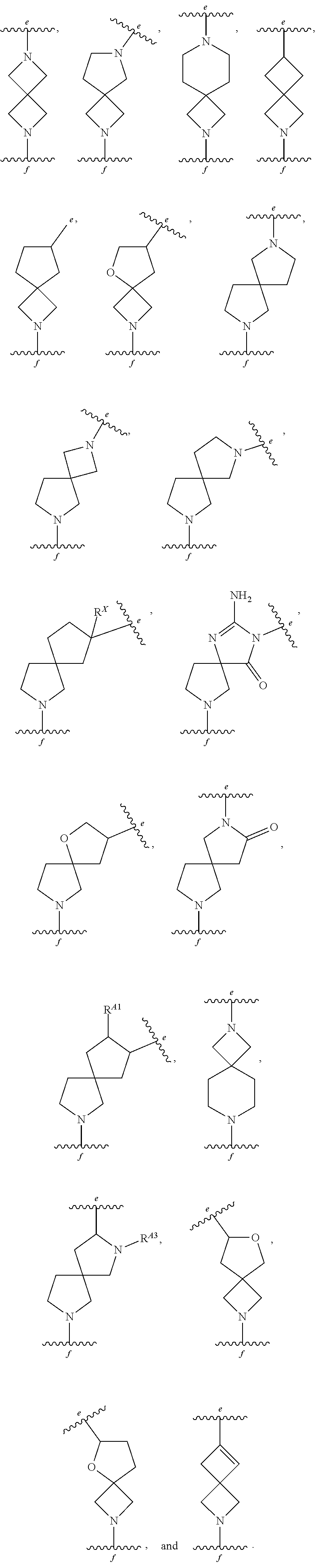

4. A compound of Formula II, 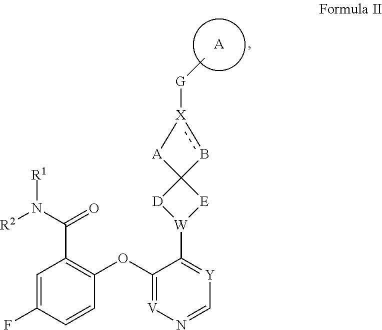
a stereoisomer, or a pharmaceutically acceptable salt thereof, wherein: [symbol]
denotes a single bond or a double bond, as valency permits;
A, B, D, and E are each independently selected from —O—, —C(R^{A1})(R^{A2})—, —C(R^{A1})(R^{A2})—C(R^{A1})(R^{A2})—, —C(R^{A1})(R^{A2})—, —C(R^{A1})(R^{A2})—NR^{A3}—, —C(═O)—, —C(R^{A})(R^{A2})—C(═O)—, and —N═C(NH₂)—, wherein no more than one of A, B, D, and E is —C(R^{A1})(R^{A2})—C(R^{A1})(R^{A2})—NR^{A3}—, —C(R^{A1})(R^{A2})C(═O)—, —C(═O)—, or N═C(NH₂)—;
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
when G is absent, X is connected with a nitrogen of Ring A;
when G is CH₂, G is connected with the nitrogen of Ring A;
W is N or CR^{W}, wherein R^{W} is H, halo, CN, OH, C₁₋₆ alkyl, C₁₋₆alkoxy, amino, C₁₋₆alkyl amino, or C₂₋₈ dialkylamino;
X is N or CR^{X}, wherein R^{X} is absent, H, halo, CN, OH, C₁₋₆ alkyl, C₁₋₆ alkoxy, amino, C₁₋₆ alkyl amino, or C₂₋₈ dialkylamino;
each R^{A1} is independently selected from absent, H, halo, C₁₋₆alkyl, C₁₋₆alkoxy, C₁₋₆ haloalkyl, C₁₋₆haloalkoxy, amino, C₁₋₆alkylamino, C₂₋₈ dialkylamino, CN, NO₂, and OH;
each R^{A2} is independently selected from H, halo, C₁₋₆alkyl, C₁₋₆ alkoxy, C₁₋₆ haloalkyl, C₁₋₆ haloalkoxy, amino, C₁₋₆ alkylamino, C₂₋₈ dialkylamino, CN, NO, and OH;
each R^{A3} is independently selected from H, C₁₋₆ alkyl, C₁₋₆ alkoxy, C₁₋₆ haloalkyl, C(O)R^{z}, and C(O)OR^{z}, wherein said C₁₋₆alkyl is optionally substituted with phenyl, C₁₋₆alkoxy, C₁₋₆ haloalkoxy, CN, NO₂, or OH;
R^{z} is H, C₁₋₆ alkyl, or phenyl;
R¹ is
(a) H, C₁₋₆ alkyl, C₁₋₆ haloalkyl, C₁₋₆ alkoxy, C₂₋₆ alkenyl, C₂₋₆ alkynyl, C₆₋₁₀aryl, C₃₋₂ cycloalkyl, 5-10 membered heteroaryl, 3-12 membered heterocycloalkyl, —C₆₋₁₀aryl-C₁₋₆ alkyl, —C₃₋₁₂cycloalkyl-C₁₋₆ alkyl, -(5-10 membered heteroaryl)-C₁₋₆ alkyl, or -(4-10 membered heterocycloalkyl)-C₁₋₆ alkyl, wherein said Cia alkyl, C₁₋₆haloalkyl, C₁₋₆ alkoxy, C₂₋₆ alkenyl, C₂₋₆alkynyl, C₆₋₁₀aryl, C₃₋₁₂ cycloalkyl, 5-10 membered heteroaryl, 3-12 membered heterocycloalkyl, —C₆₋₁₀aryl-C₁₋₆alkyl, —C₃₋₁₂cycloalkyl-C₁₋₆alkyl, -(5-10 membered heteroaryl)-C₁₋₆alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
(c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1as};
each R^{1as} is independently halo, oxo, C₁₋₆ alkyl, C-haloalkyl, C(═O)(R^{3a}′), N(R^{3a}′)(R^{3b}′), or S(═)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C2-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

5. A compound of Formula II-a, 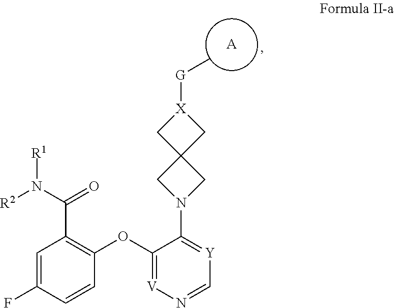
a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R—;
G is absent or CH₂, wherein:
when G is absent, X is connected with a nitrogen of Ring A;
when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
(a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
(c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a};
each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}), OC(O)N(R^{4a})(R^{4b}), S(═O)₂R^{4a}, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

6. A compound of Formula II-b, 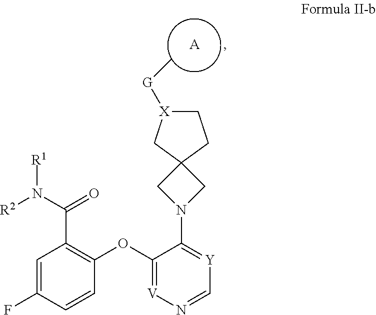
a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
when G is absent, X is connected with a nitrogen of Ring A;
when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
(a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
(c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a};
each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

7. A compound of Formula II-c, 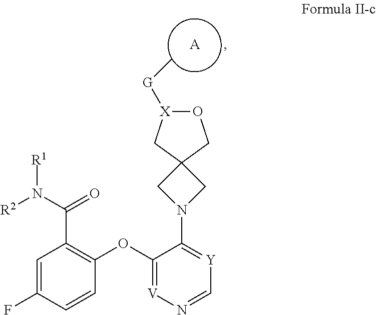
a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
when G is absent, X is connected with a nitrogen of Ring A;
when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₄alkyl amino, or C₂₋₈ dialkylamino;
R¹ is
(a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
(c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C2-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

8. A compound of Formula II-d, 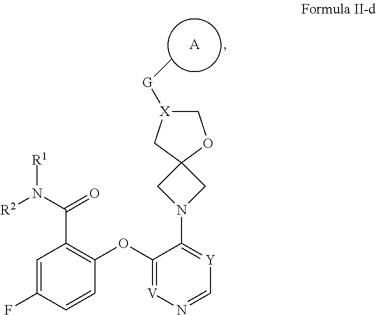
a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
when G is absent, X is connected with a nitrogen of Ring A;
when G is CH₂, G is connected with the nitrogen of Ring A;
X is N or CR^{X}, wherein R^{X} is H, halo, CN, OH, C₁₋₆ alkyl, C₁₋₆ alkoxy, amino, C₁₋₆ alkyl amino, or C₂₋₈ dialkylamino;
R¹ is
(a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
(c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a};
each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′, C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

9. A compound of Formula II-e, 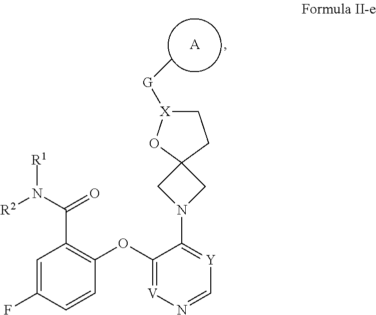
a stereoisomer, or a pharmaceutically acceptable salt thereof, wherein:
V is N or CH;
Y is N or CH;
Ring A is 4-18 membered heterocycloalkyl or 5- to 10-membered heteroaryl, each of which has at least one nitrogen and optionally one or more other heteroatom ring members, wherein the 4-18 membered heterocycloalkyl or the 5- to 10-membered heteroaryl is optionally substituted with one or more R³;
G is absent or CH₂, wherein:
when G is absent, X is connected with a nitrogen of Ring A;
when G is CH₂, G is connected with the nitrogen of Ring A;
(a) H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(4-10 membered heterocycloalkyl)-C₁₋₆ alkyl, wherein said C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(4-10 membered heterocycloalkyl)-C₁₋₆ alkyl are each optionally substituted with 1, 2, 3, 4, or 5 substituents independently selected from R^{g};
(b) 3- to 12-membered heterocycloalkyl comprising a sulfur atom, wherein the sulfur atom of the 3- to 12-membered heterocycloalkyl is optionally substituted with one or two R^{1s};
each R^{1s} is independently oxo or ═NR^{4a};
(c) —C₁₋₆ alkylene-N(R^{3a}′)(R^{3b}′) or —C₃₋₁₂ cycloalkylene-N(R^{3a}′)(R^{3b}′), wherein the C₁₋₆ alkylene or C₃₋₁₂ cycloalkylene is optionally substituted with one or more R^{1a} each R^{1as} is independently halo, oxo, C₁₋₆ alkyl, C₁₋₆ haloalkyl, C(═O)(R^{3a}′) N(R^{3a}′)(R^{3b}′) or S(═O)₂R^{3a}′;
(d) 3- to 12-membered heterocycloalkyl which has at least one nitrogen and optionally one or more other heteroatom ring members, wherein the 3- to 12-membered heterocycloalkyl is optionally substituted with one or more R^{1bs};
each R^{1bs} is independently halo, oxo, OR^{3a}′, C₁₋₆ alkyl, C₁₋₆ haloalkyl, C₃₋₁₂ cycloalkyl, 3- to 12-membered heterocycloalkyl, C(═O)(R^{3a}′), N(R^{3a}′)(R^{3b}′), or S(═O)₂R^{3a}′; or
(e) C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′) N(R^{3a}′)(R^{3b}′), OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl;
each R^{3c}′ is independently H, C₁₋₆ alkyl, C₁₋₆ haloalkyl, C₃₋₁₂ cycloalkyl, or 3- to 12-membered heterocycloalkyl;
R² is H, C₁₋₆ alkyl, C₁₋₆ haloalkyl, C₁₋₆ alkoxy, C₂₋₆ alkenyl, C₂₋₆ alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, or -(3-12 membered heterocycloalkyl)-C₁₋₆ alkyl, wherein said C₁₋₆ alkyl, C₁₋₆ haloalkyl, C₁₋₆ alkoxy, C₂₋₆ alkenyl, C2-6 alkynyl, C₆₋₁₀ aryl, C₃₋₁₂ cycloalkyl, 5-10 membered heteroaryl, 3-12 membered heterocycloalkyl, —C₆₋₁₀ aryl-C₁₋₆ alkyl, —C₃₋₁₂ cycloalkyl-C₁₋₆ alkyl, -(5-10 membered heteroaryl)-C₁₋₆ alkyl, and -(3-12 membered heterocycloalkyl)-C₁₋₆ alkyl are each optionally substituted with 1, 2, 3, 4, or 5 substituents independently selected from R^{g}; or
R¹ and R² optionally form a 3- to 12-membered heterocycloalkyl with the nitrogen to which they are connected, wherein the heterocycloalkyl is optionally substituted with one or more C₁₋₆ alkyl, halo, OH, CN, or C₁₋₆ alkoxy;
each R^{g} is independently selected from OH, NO₂, CN, halo, oxo, OBn, N(R_{N})₂, C₁₋₆ alkyl, C₆₋₁₀ aryl, 5- to 10-membered heteroaryl, N(R^{4a}′)(R^{4b}′), C₂₋₆ alkenyl, C₂₋₆ alkynyl, C₁₋₆ haloalkyl, C₃₋₆ cycloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thiol, C₁₋₆ alkylthio, C₁₋₆ alkylsulfinyl, C₁₋₆ alkylsulfonyl, carboxy, aminocarbonyl, C₁₋₆ alkylcarbonyl, and C₁₋₆ alkoxycarbonyl;
each R³ is independently H, oxo, N(R^{4a})(R^{4b}), C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, 3- to 12-membered heterocycloalkyl, C(O)(R^{4a}), C(O)(OR^{4a}), or C(O)O—C(R^{4a})₂—OC(O)(R^{4a}), wherein the C₁₋₆ alkyl, C₂₋₆ alkenyl, C₂₋₆ alkynyl, C₁₋₆ alkoxy, C₃₋₁₂ cycloalkyl, C₆₋₁₀ aryl, 5- to 10-membered heteroaryl, or 3- to 12-membered heterocycloalkyl is optionally substituted with one or more R^{3a};
each R^{3a} is independently halo, OR^{4a}, oxo, C(O)O(R^{4a}), CN, C(═O)N(R^{4a})(R^{4b}) OC(O)N(R^{4a})(R^{4b}), S(═O)₂R^{4b}′, N(R^{4a})(R^{4b}), C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl, wherein the C₁₋₆ alkyl, C₃₋₁₂ cycloalkyl, C₁₋₆ alkoxy, C₆₋₁₀ aryl, 3- to 12-membered heterocycloalkyl, or 5- to 10-membered heteroaryl is optionally substituted with one or more substituent independently selected from halo, O(R^{4a}′), oxo, CN, C(O)OR^{4a}′, OC(O)N(R^{4a}′)(R^{4b}′), N(R^{4a}′)(R^{4b}′), N(R^{4a}′)C(O)(R^{4b}′), N(R^{4a}′)C(O)O(R^{4b}′), C₁₋₆ alkyl that is optionally substituted with O(R^{4a}), and 3- to 12-membered heterocycloalkyl that is optionally substituted with oxo;
each R^{4a} is independently H, CN, or C₁₋₆ alkyl optionally substituted with one or more halo, C₆₋₁₀ aryl, or N(R^{4a}′)(R^{4b}′);
each R^{4b} is independently H, S(═O)₂R^{4b}′, C(O)OR^{4b}′, C(O)R^{4b}′, C(O)NR^{4a}′R^{4b}′ C(O)CH₂—N(R^{4a}′)(R^{4b}′), C₁₋₆ alkyl, C₃₋₁₂ cycloalkyl, C₆₋₁₀ aryl, 5- to 10-membered heteroaryl, or 3- to 12-membered heterocycloalkyl, wherein the C₁₋₆ alkyl is optionally substituted with C₁₋₆ alkoxy or C(O)N(R^{4a}′)(R^{4b}′);
each R^{4a}′ and R^{4b}′ is independently H, C₁₋₆ alkyl, C₃₋₁₂ cycloalkyl, or 3- to 12-membered heterocycloalkyl; and
each R_{N} is independently H, C₁₋₆ alkyl, or C₁₋₆ haloalkyl;
wherein any cycloalkyl or heterocycloalkyl group is optionally further substituted with 1 or 2 oxo groups.

10. The compound of any one of the preceding claims, wherein V is N.

11. The compound of any one of the preceding claims, wherein V is CH.

12. The compound of any one of the preceding claims, wherein Y is N.

13. The compound of any one of the preceding claims, wherein Y is CH.

14. The compound of any one of the preceding claims, wherein G is absent and X is CH which is connected with a nitrogen of Ring A.

15. The compound of any one of the preceding claims, wherein G is CH₂ and G is connected with the nitrogen of Ring A.

16. The compound of any one of the preceding claims, wherein W is N.

17. The compound of any one of the preceding claims, wherein R¹ is C₁₋₆ alkyl or C₁-C₆ haloalkyl.

18. The compound of any one of the preceding claims, wherein R¹ is ethyl, isopropyl, or —CH₂—CHF₂.

19. The compound of any one of the preceding claims, wherein R¹ is C₃₋₁₂ cycloalkyl substituted with one or more 3- to 12-membered heterocycloalkyl which has at least one nitrogen and optionally one or more other heteroatom ring members; and wherein each of the C₃₋₁₂ cycloalkyl and the 3- to 12-membered heterocycloalkyl is optionally substituted with one or more R^{1cs};
each R^{1cs} is independently halo, oxo, C₁₋₆ alkyl, C₁₋₆ haloalkyl, C(═O)(R^{3a}′), N(R^{3a}′)(R^{3b}′) OR^{3a}′, or S(═O)₂R^{3a}′, wherein the C₁₋₆ alkyl or C₁₋₆ haloalkyl is optionally substituted with one or more C₁₋₆ alkoxy;
each R^{3a}′ is independently H, C₁₋₆ alkyl, C₁₋₆ haloalkyl, C(O)R^{3c}′, C₃₋₁₂ cycloalkyl, or 3- to 12-membered heterocycloalkyl, wherein the C₁₋₆ alkyl, C₁₋₆ haloalkyl, C₃₋₁₂ cycloalkyl, or 3- to 12-membered heterocycloalkyl are optionally substituted with C₁₋₆ alkoxy;
each R^{3b}′ is independently H, C₁₋₆ alkyl, C₁₋₆ haloalkyl, C₃₋₁₂ cycloalkyl, or 3- to 12-membered heterocycloalkyl; and
each R^{3c}′ is independently H, C₁₋₆ alkyl, C₁₋₆ haloalkyl, C₃₋₁₂ cycloalkyl, or 3- to 12-membered heterocycloalkyl.

20. The compound of any one of the preceding claims, wherein R¹ is

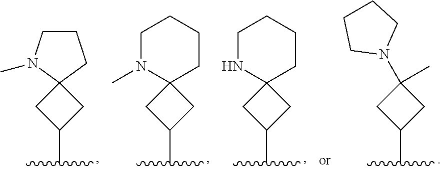

21. The compound of any one of the preceding claims, wherein R¹ and R² form a 6-membered heterocycloalkyl with the nitrogen to which they are connected, wherein the heterocycloalkyl is substituted with one or more C₁₋₆ alkyl, halo, OH, CN, or C₁₋₆ alkoxy.

22. The compound of any one of the preceding claims, wherein R² is C₁₋₆ alkyl.

23. The compound of any one of the preceding claims, wherein R² is isopropyl.

24. The compound of any one of the preceding claims, wherein R¹ and R² form 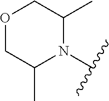 [SMILES: *N1C(C)COCC1C]
group with the nitrogen to which they are connected.

25. The compound of any one of the preceding claims, wherein Ring A is 4-18 membered heterocycloalkyl having at least one nitrogen and optionally one or more other heteroatom ring members, wherein the 4-18 membered heterocycloalkyl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}), OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

26. The compound of any one of the preceding claims, wherein Ring A is

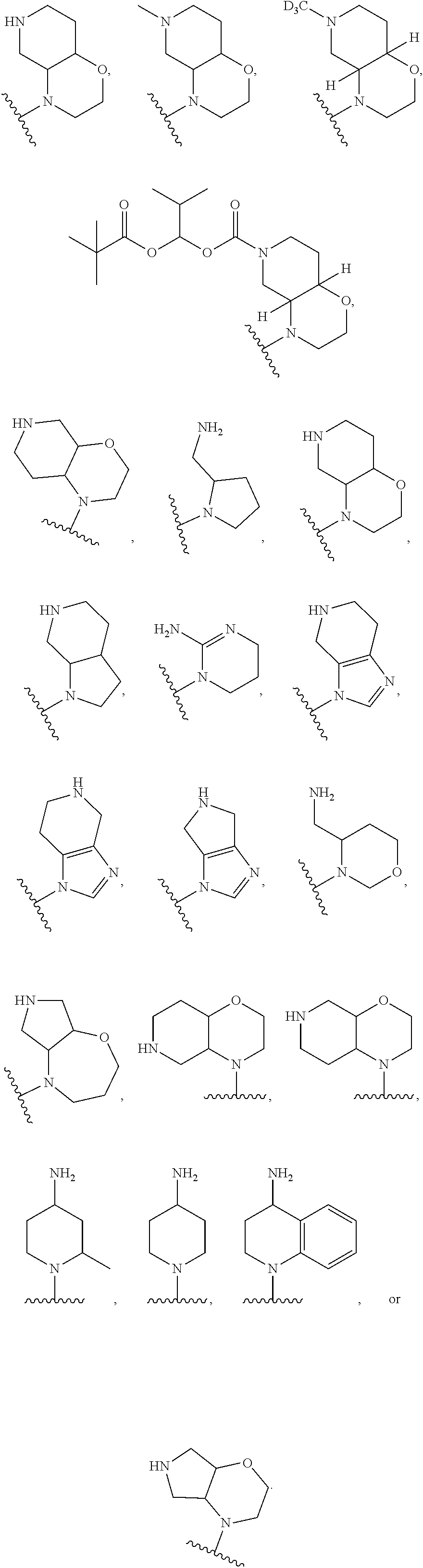

27. The compound of any one of the preceding claims, wherein Ring A is 5- to 10-membered heteroaryl having at least one nitrogen and optionally one or more other heteroatom ring members, wherein the 5- to 10-membered heteroaryl is optionally substituted with one or more R³;
each R³ is independently H, oxo, N(R^{4a})(R^{4b}), C₁₋₆ alkyl, C₂₋₆ alkenyl, C₂₋₆ alkynyl, or C₁₋₆ alkoxy, wherein the C₁₋₆ alkyl, C₂₋₆ alkenyl, C₂₋₆ alkynyl, or C₁₋₆ alkoxy is optionally substituted with one or more R^{3a};
each R^{3a} is independently halo, OR^{4a}, oxo, C(O)O(R^{4a}), CN, C(═O)N(R^{4a})(R^{4b}), OC(O)N(R^{4a})(R^{4b}), or N(R^{4a})(R^{4b});
each R^{4a} is independently H or C₁₋₆ alkyl; and
each R^{4b} is independently H or C₁₋₆ alkyl.

28. The compound of any one of the preceding claims, wherein Ring A is

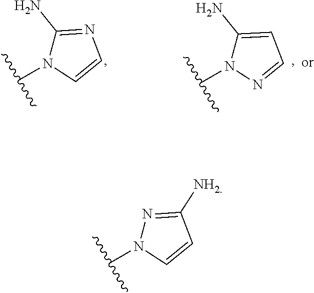 [SMILES: *n1ccnc1N ; *n1nccc1N ; *n1ccc(N)n1]

29. The compound of any one of the preceding claims, wherein each R³ is independently H, methyl, —CH₂—NH₂, oxo, or —NH₂.

30. A compound as shown in Table 1 or a stereoisomer thereof, or a pharmaceutically acceptable salt thereof.

31. A compound as shown in Table 1 or a pharmaceutically acceptable salt thereof.

32. A compound as shown in Table 1.

33. A compound as shown in Table 1 or a pharmaceutically acceptable salt thereof, wherein the salt is hydrochloride.

34. The compound according to any one of the preceding claims, wherein the compound is useful for the treatment of cancer and wherein the compound minimizes hERG binding.

35. A pharmaceutical composition comprising a compound of any one of the preceding claims, or a pharmaceutically acceptable salt thereof, and at least one pharmaceutically acceptable carrier.

36. A pharmaceutical composition comprising a salt or crystalline form of the compound of any one of claims 1-34, and at least one pharmaceutically acceptable carrier.

37. A method of inhibiting the interaction between menin and MLL comprising contacting the menin and MLL with a compound of any one of claims 1-34 or a pharmaceutical composition of claim 35 or claim 36.

38. A method of treating cancer in a patient comprising administering to the patient a compound of any one of claims 1-34 or a pharmaceutical composition of claim 35 or claim 36.

39. The method of claim 38, wherein the cancer is a hematological cancer.

40. The method of claim 38, wherein the cancer is a leukemia or lymphoma.

41. The method of claim 38, wherein the cancer is mixed lineage leukemia (MLL), MLL-related leukemia, MLL-associated leukemia, MLL-positive leukemia, MLL-induced leukemia, rearranged mixed lineage (KMT2A-rearranged) leukemia (MLL-r), leukemia associated with a MLL rearrangement or a rearrangement of the MLL gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL), acute myeloid leukemia (AML), acute granulocytic leukemia, acute nonlymphocytic leukemia, chronic lymphocytic leukemia (CLL), chronic myelogenous leukemia (CML), therapy related leukemia, myelodysplastic syndrome (MDS), myeloproliferative disease (MPD), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sézary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non Hodgkin's lymphoma (malignant lymphoma), or Waldenstrom's macroglobulinemia.

42. The method of claim 38, wherein the cancer is an abstract nucleophosmin (NPM1)-mutated acute myeloid leukemia (i.e., NPM1^{mut} acute myloid leukemia) or a rearranged mixed lineage (KMT2A-rearranged) leukemia (MLL-r).

43. The compound of any one of claims 1-34 or a pharmaceutically acceptable salt thereof, or the pharmaceutical composition of claim 35 or claim 36, for use in treating or preventing a disease caused by, or associated with, menin expression, activity, and/or function.

44. The compound of any one of claims 1-34 or a pharmaceutically acceptable salt thereof, or the pharmaceutical composition of claim 35 or claim 36, for use in treating or preventing cancer.

45. Use of the compound of any one of claims 1-34 or a pharmaceutically acceptable salt thereof, or the pharmaceutical composition of claim 35 or claim 36, for treating or preventing a disease caused by, or associated with, menin expression, activity, and/or function.

46. Use of the compound of any one of claims 1-34 or a pharmaceutically acceptable salt thereof, or the pharmaceutical composition of claim 35 or claim 36, in the manufacture of a medicament for treating or preventing a disease caused by, or associated with, menin expression, activity, and/or function.

47. Use of a compound of any of claims 1-34 or a pharmaceutically acceptable salt thereof, or the pharmaceutical composition of claim 35 or claim 36, for treating or preventing cancer.

48. Use of the compound of any one of claims 1-34 or a pharmaceutically acceptable salt thereof, or the pharmaceutical composition of claim 35 or claim 36, in the manufacture of a medicament for treating or preventing cancer.

49. A kit comprising the compound of any one of claims 1-34 or a pharmaceutically acceptable salt thereof, or the pharmaceutical composition of claim 35 or claim 36 and instructions for its use.

50. A method of preparing a compound herein according to a scheme of the present disclosure or synthetic description in the Examples.

51. An intermediate useful in the preparation of any one of the compounds herein.
